(19) United States
(12) Patent Application Publication
(10) Pub. No.: US 2004/0018555 A1
(43) Pub. Date: Jan. 29, 2004

(54) Novel antibodies that bind to antigenic polypeptides, nucleic acids encoding the antigens, and methods of use

(76) Inventors: David W. Anderson, Branford, CT (US); Bryan D. Zerhusen, Branford, CT (US); Li Li, Branford, CT (US); Mei Zhong, Branford, CT (US); Stacie J. Casman, North Haven, CT (US); Valerie Gerlach, Branford, CT (US); Richard A. Shimkets, Guilford, CT (US); Linda Gorman, Branford, CT (US); Carol E. A. Pena, Guilford, CT (US); Ramesh Kekuda, Norwalk, CT (US); Meera Patturajan, Branford, CT (US); Kimberly A. Spytek, New Haven, CT (US); Mario W. Leite, Milford, CT (US); Luca Rastelli, Guilford, CT (US); John R. MacDougall, Hamden, CT (US); Raymond J. Taupier JR., East Haven, CT (US); Xiaojia Sasha Guo, Branford, CT (US); Charles E. Miller, Guilford, CT (US); Suresh G. Shenoy, Branford, CT (US); Tord Hjalt, Lomma, CT (US); Edward Z. Voss, Wallingford, CT (US); Ferenc L. Boldog, North Haven, CT (US); Uriel M. Malyankar, Branford, CT (US); Muralidhara Padigaru, Branford, CT (US); Weizhen Ji, Branford, CT (US); Glennda Smithson, Guilford, CT (US); Shlomit R. Edinger, New Haven, CT (US); Isabelle Millet, Milford, CT (US); Karen Ellerman, Branford, CT (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/161,493

(22) Filed: Jun. 3, 2002

Related U.S. Application Data
(60) Provisional application No. 60/295,607, filed on Jun. 4, 2001.
(60) Provisional application No. 60/337,524, filed on Nov. 16, 2001.
(60) Provisional application No. 60/296,404, filed on Jun. 6, 2001.
(60) Provisional application No. 60/296,418, filed on Jun. 6, 2001.
(60) Provisional application No. 60/296,575, filed on Jun. 7, 2001.
(60) Provisional application No. 60/359,151, filed on Feb. 21, 2002.
(60) Provisional application No. 60/297,414, filed on Jun. 11, 2001.
(60) Provisional application No. 60/297,573, filed on Jun. 12, 2001.
(60) Provisional application No. 60/341,143, filed on Dec. 14, 2001.
(60) Provisional application No. 60/297,567, filed on Jun. 12, 2001.
(60) Provisional application No. 60/318,771, filed on Sep. 12, 2001.
(60) Provisional application No. 60/298,285, filed on Jun. 14, 2001.
(60) Provisional application No. 60/298,528, filed on Jun. 15, 2001.
(60) Provisional application No. 60/325,687, filed on Sep. 28, 2001.
(60) Provisional application No. 60/298,556, filed on Jun. 15, 2001.
(60) Provisional application No. 60/299,133, filed on Jun. 18, 2001.
(60) Provisional application No. 60/299,230, filed on Jun. 19, 2001.
(60) Provisional application No. 60/358,643, filed on Feb. 21, 2002.
(60) Provisional application No. 60/299,949, filed on Jun. 21, 2001.
(60) Provisional application No. 60/300,177, filed on Jun. 22, 2001.
(60) Provisional application No. 60/361,964, filed on Mar. 5, 2002.
(60) Provisional application No. 60/361,195, filed on Feb. 28, 2002.
(60) Provisional application No. 60/371,523, filed on Apr. 10, 2002.
(60) Provisional application No. 60/301,530, filed on Jun. 28, 2001.
(60) Provisional application No. 60/371,346, filed on Apr. 10, 2002.
(60) Provisional application No. 60/301,550, filed on Jun. 28, 2001.
(60) Provisional application No. 60/302,951, filed on Jul. 3, 2001.
(60) Provisional application No. 60/339,266, filed on Oct. 24, 2001.

(51) Int. Cl. A61K 39/395; G01N 33/53; C07H 21/04; C12P 21/02; C12N 5/06; C07K 16/40

(52) U.S. Cl. 435/7.1; 424/130.1; 435/69.1; 435/320.1; 435/326; 530/388.1; 536/23.53

(57) ABSTRACT

Disclosed herein are nucleic acid sequences that encode polypeptides. Also disclosed are antibodies, which immunospecifically-bind to the polypeptide, as well as derivatives, variants, mutants, or fragments of the aforementioned polypeptide, polynucleotide, or antibody. The invention further discloses therapeutic, diagnostic and research methods for diagnosis, treatment, and prevention of disorders involving any one of these novel human nucleic acids, polypeptides, or antibodies, or fragments thereof.

### RELATED APPLICATIONS

This application claims priority to U.S. Ser. No. 60/295,607 filed Jun. 4, 2001; U.S. Ser. No. 60/337,524 filed Nov. 16, 2001; U.S. Ser. No. 60/296,404 filed Jun. 6, 2001; U.S. Ser. No. 60/296,418 filed Jun. 6, 2001; U.S. Ser. No. 60/296,575 filed Jun. 7, 2001; U.S. Ser. No. 60/359,151 filed Feb. 21, 2002; U.S. Ser. No. 60/297,414 filed Jun. 11, 2001; U.S. Ser. No. 60/297,573 filed Jun. 12, 2001; U.S. Ser. No. 60/341,143 filed Dec. 14, 2001; U.S. Ser. No. 60/297,567 filed Jun. 12, 2001; U.S. Ser. No. 60/318,771 filed Sep. 12, 2001; U.S. Ser. No. 60/298,285 filed Jun. 14, 2001; U.S. Ser. No. 60/298,528 filed Jun. 15, 2001; U.S. Ser. No. 60/325,685 filed Sep. 27, 2001; U.S. Ser. No. 60/298,556 filed Jun. 15, 2001; U.S. Ser. No. 60/299,133 filed Jun. 18, 2001; U.S. Ser. No. 60/299,230 filed Jun. 19, 2001; U.S. Ser. No. 60/358,643 filed Feb. 21, 2002; U.S. Ser. No. 60/299,949 filed Jun. 21, 2001; U.S. Ser. No. 60/300,177 filed Jun. 22, 2001; U.S. Ser. No. 60/361,964 filed Mar. 5, 2002; U.S. Ser. No. 60/361,195 filed Feb. 28, 2002; U.S. Ser. No. 60/371,523 filed Apr. 10, 2002; U.S. Ser. No. 60/301,530 filed Jun. 28, 2001; U.S. Ser. No. 60/371,346 filed Apr. 10, 2002; U.S. Ser. No. 60/301,550 filed Jun. 28, 2001; U.S. Ser. No. 60/302,951 filed Jul. 3, 2001; U.S. Ser. No. 60/339,266 filed Oct. 24, 2001, each of which is incorporated herein by reference in its entirety.

### FIELD OF THE INVENTION

The present invention relates to novel antibodies that bind immunospecifically to antigenic polypeptides, wherein the polypeptides have characteristic properties related to biochemical or physiological responses in a cell, a tissue, an organ or an organism. The novel polypeptides are gene products of novel genes, or are specified biologically active fragments or derivatives thereof. Methods of use of the antibodies encompass procedures for diagnostic and prognostic assay of the polypeptides, as well as methods of treating diverse pathological conditions.

### BACKGROUND OF THE INVENTION

Eukaryotic cells are characterized by biochemical and physiological processes which under normal conditions are exquisitely balanced to achieve the preservation and propagation of the cells. When such cells are components of multicellular organisms such as vertebrates, or more particularly organisms such as mammals, the regulation of the biochemical and physiological processes involves intricate signaling pathways. Frequently, such signaling pathways involve extracellular signaling proteins, cellular receptors that bind the signaling proteins, and signal transducing components located within the cells.

Signaling proteins may be classified as endocrine effectors, paracrine effectors or autocrine effectors. Endocrine effectors are signaling molecules secreted by a given organ into the circulatory system, which are then transported to a distant target organ or tissue. The target cells include the receptors for the endocrine effector, and when the endocrine effector binds, a signaling cascade is induced. Paracrine effectors involve secreting cells and receptor cells in close proximity to each other, for example two different classes of cells in the same tissue or organ. One class of cells secretes the paracrine effector, which then reaches the second class of cells, for example by diffusion through the extracellular fluid. The second class of cells contains the receptors for the paracrine effector; binding of the effector results in induction of the signaling cascade that elicits the corresponding biochemical or physiological effect. Autocrine effectors are highly analogous to paracrine effectors, except that the same cell type that secretes the autocrine effector also contains the receptor. Thus the autocrine effector binds to receptors on the same cell, or on identical neighboring cells. The binding process then elicits the characteristic biochemical or physiological effect.

Signaling processes may elicit a variety of effects on cells and tissues including by way of nonlimiting example induction of cell or tissue proliferation, suppression of growth or proliferation, induction of differentiation or maturation of a cell or tissue, and suppression of differentiation or maturation of a cell or tissue.

Many pathological conditions involve dysregulation of expression of important effector proteins. In certain classes of pathologies the dysregulation is manifested as elevated or excessive synthesis and secretion of protein effectors. In a clinical setting a subject may be suspected of suffering from a condition brought on by elevated or excessive levels of a protein effector of interest.

Antibodies are multichain proteins that bind specifically to a given antigen, and bind poorly, or not at all, to substances deemed not to be cognate antigens. Antibodies are comprised of two short chains termed light chains and two long chains termed heavy chains. These chains are constituted of immunoglobulin domains, of which generally there are two classes: one variable domain per chain, one constant domain in light chains, and three or more constant domains in heavy chains. The antigen-specific portion of the immunoglobulin molecules resides in the variable domains; the variable domains of one light chain and one heavy chain associate with each other to generate the antigen-binding moiety. Antibodies that bind immunospecifically to a cognate or target antigen bind with high affinities. Accordingly, they are useful in assaying specifically for the presence of the antigen in a sample. In addition, they have the potential of inactivating the activity of the antigen.

Therefore there is a need to assay for the level of a protein effector of interest in a biological sample from such a subject, and to compare this level with that characteristic of a nonpathological condition. In particular, there is a need for such an assay based on the use of an antibody that binds immunospecifically to the antigen. There further is a need to inhibit the activity of the protein effector in cases where a pathological condition arises from elevated or excessive levels of the effector based on the use of an antibody that binds immunospecifically to the effector. Thus, there is a need for the antibody as a product of manufacture. There further is a need for a method of treatment of a pathological condition brought on by an elevated or excessive level of the protein effector of interest based on administering the antibody to the subject.

### SUMMARY OF THE INVENTION

The invention is based in part upon the discovery of nucleic acid sequences encoding novel polypeptides. The novel nucleic acids and polypeptides are referred to herein as NOVX, or NOV1, NOV2, NOV3, etc., nucleic acids and polypeptides. These nucleic acids and polypeptides, as well as derivatives, homologs, analogs and fragments thereof, will hereinafter be collectively designated as “NOVX” nucleic acid or polypeptide sequences.

In one aspect, the invention provides an isolated polypeptide comprising a mature form of a NOVX amino acid. The polypeptide can be, for example, a NOVX amino acid sequence or a variant of a NOVX amino acid sequence, wherein any amino acid specified in the chosen sequence is changed to a different amino acid, provided that no more than 15% of the amino acid residues in the sequence are so changed. The invention also includes fragments of any of NOVX polypeptides. In another aspect, the invention also includes an isolated nucleic acid that encodes a NOVX polypeptide, or a fragment, homolog, analog or derivative thereof.

Also included in the invention is a NOVX polypeptide that is a naturally occurring variant of a NOVX sequence. In one embodiment, the variant includes an amino acid sequence that is the translation of a nucleic acid sequence differing by a single nucleotide from a NOVX nucleic acid sequence. In another embodiment, the NOVX polypeptide is a variant polypeptide described therein, wherein any amino acid specified in the chosen sequence is changed to provide a conservative substitution.

In another aspect, invention provides a method for determining the presence or amount of the NOVX polypeptide in a sample by providing a sample; introducing the sample to an antibody that binds immunospecifically to the polypeptide; and determining the presence or amount of antibody bound to the NOVX polypeptide, thereby determining the presence or amount of the NOVX polypeptide in the sample.

In yet another aspect, the invention includes a method for determining the presence of or predisposition to a disease associated with altered levels of a NOVX polypeptide in a mammalian subject by measuring the level of expression of the polypeptide in a sample from the first mammalian subject; and comparing the amount of the polypeptide in the sample of the first step to the amount of the polypeptide present in a control sample from a second mammalian subject known not to have, or not to be predisposed to, the disease. An alteration in the expression level of the polypeptide in the first subject as compared to the control sample indicates the presence of or predisposition to the disease.

In another aspect, the invention includes pharmaceutical compositions that include therapeutically- or prophylactically-effective amounts of a therapeutic and a pharmaceutically-acceptable carrier. The therapeutic can be, e.g., a NOVX nucleic acid, a NOVX polypeptide, or an antibody specific for a NOVX polypeptide. In a further aspect, the invention includes, in one or more containers, a therapeutically- or prophylactically-effective amount of this pharmaceutical composition.

In still another aspect, the invention provides the use of a therapeutic in the manufacture of a medicament for treating a syndrome associated with a human disease that is associated with a NOVX polypeptide.

In a further aspect, the invention provides a method for modulating the activity of a NOVX polypeptide by contacting a cell sample expressing the NOVX polypeptide with antibody that binds the NOVX polypeptide in an amount sufficient to modulate the activity of the polypeptide.

The invention also includes an isolated nucleic acid that encodes a NOVX polypeptide, or a fragment, homolog, analog or derivative thereof. In a preferred embodiment, the nucleic acid molecule comprises the nucleotide sequence of a naturally occurring allelic nucleic acid variant. In another embodiment, the nucleic acid encodes a variant polypeptide, wherein the variant polypeptide has the polypeptide sequence of a naturally occurring polypeptide variant. In another embodiment, the nucleic acid molecule differs by a single nucleotide from a NOVX nucleic acid sequence. In one embodiment, the NOVX nucleic acid molecule hybridizes under stringent conditions to the nucleotide sequence selected from the group consisting of SEQ ID NO: 2n−1, wherein n is an integer between 1 and 73, or a complement of the nucleotide sequence. In one embodiment, the invention provides a nucleic acid molecule wherein the nucleic acid includes the nucleotide sequence of a naturally occurring allelic nucleic acid variant.

Also included in the invention is a vector containing one or more of the nucleic acids described herein, and a cell containing the vectors or nucleic acids described herein. The invention is also directed to host cells transformed with a vector comprising any of the nucleic acid molecules described above.

In yet another aspect, the invention provides for a method for determining the presence or amount of a nucleic acid molecule in a sample by contacting a sample with a probe that binds a NOVX nucleic acid and determining the amount of the probe that is bound to the NOVX nucleic acid. For example the NOVX nucleic may be a marker for cell or tissue type such as a cell or tissue type that is cancerous.

In yet a further aspect, the invention provides a method for determining the presence of or predisposition to a disease associated with altered levels of a nucleic acid molecule in a first mammalian subject, wherein an alteration in the level of the nucleic acid in the first subject as compared to the control sample indicates the presence of or predisposition to the disease.

The invention further provides an antibody that binds immunospecifically to a NOVX polypeptide. The NOVX antibody may be monoclonal, humanized, or a fully human antibody. Preferably, the antibody has a dissociation constant for the binding of the NOVX polypeptide to the antibody less than 1×10⁻⁹ M. More preferably, the NOVX antibody neutralizes the activity of the NOVX polypeptide.

In a further aspect, the invention provides for the use of a therapeutic in the manufacture of a medicament for treating a syndrome associated with a human disease, associated with a NOVX polypeptide. Preferably the therapeutic is a NOVX antibody.

In yet a further aspect, the invention provides a method of treating or preventing a NOVX-associated disorder, a method of treating a pathological state in a mammal, and a method of treating or preventing a pathology associated with a polypeptide by administering a NOVX antibody to a subject in an amount sufficient to treat or prevent the disorder.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the invention will be apparent from the following detailed description and claims.

### DETAILED DESCRIPTION OF THE INVENTION

The present invention provides novel nucleotides and polypeptides encoded thereby. Included in the invention are the novel nucleic acid sequences, their encoded polypeptides, antibodies, and other related compunds. The sequences are collectively referred to herein as “NOVX nucleic acids” or “NOVX polynucleotides” and the corresponding encoded polypeptides are referred to as “NOVX polypeptides” or “NOVX proteins.” Unless indicated otherwise, “NOVX” is meant to refer to any of the novel sequences disclosed herein. Table 1 provides a summary of the NOVX nucleic acids and their encoded polypeptides.

TABLE 1
NOVX Polynucleotide and Polypeptide Sequences and
Corresponding SEQ ID Numbers

| | | SEQ ID | | |
|---|---|---|---|---|
| | | NO | | |
| | | (nucleic | SEQ ID NO | |
| | Internal Identification | acid) | (polypeptide) | Homology |
| 1a | CG100653-01 | 1 | 2 | Cadherin Associated Protein-like |
| 2a | CG100689-01 | 3 | 4 | Leucine Rich Repeat-like |
| 3a | CG100760-01 | 5 | 6 | Leucine Rich Repeat-like |
| 4a | CG100851-02 | 7 | 8 | Leukocyte Surface Antigen |
| | | | | CD53-like |
| 5a | CG101068-01 | 9 | 10 | Claudin-9-like |
| 6a | CG101231-01 | 11 | 12 | Integral Membrane Protein Isoform |
| | | | | 2-like |
| 6b | CG101231-02 | 13 | 14 | Integral Membrane Protein Isoform |
| | | | | 2-like |
| 7a | CG101362-01 | 15 | 16 | Prion Protein-like |
| 8a | CG101458-01 | 17 | 18 | Von Willebrand Domain |
| | | | | Containing Protein-like |
| 9a | CG101475-01 | 19 | 20 | Plasma Membrane Protein-like |
| 9b | CG101475-02 | 21 | 22 | Plasma Membrane Protein-like |
| 10a | CG101772-01 | 23 | 24 | XAGE-like |
| 11a | CG102532-01 | 25 | 26 | Emerin-like |
| 12a | CG102575-01 | 27 | 28 | ATPase-like |
| 12b | CG102575-02 | 29 | 30 | ATPase-like |
| 13a | CG102615-01 | 31 | 32 | Mat8 (Mammary Tumor 8 kDa) |
| | | | | Protein-like |
| 13b | CG102615-04 | 33 | 34 | Mat8 (Mammary Tumor 8 kDa) |
| | | | | Protein-like |
| 14a | CG102646-01 | 35 | 36 | High Affinity Proline Permease-like |
| 15a | CG102878-01 | 37 | 38 | Transmembrane-like |
| 15b | CG102878-02 | 39 | 40 | Transmembrane-like |
| 16a | CG103459-01 | 41 | 42 | Peptide/Histidine Transporter-like |
| 17a | CG104210-01 | 43 | 44 | Type III Membrane Protein-like |
| 17b | CG104210-02 | 45 | 46 | Type III Membrane Protein-like |
| 17c | 272249075 | 47 | 48 | Type III Membrane Protein-like |
| 18a | CG104251-01 | 49 | 50 | Type III Membrane Protein-like |
| 19a | CG104934-01 | 51 | 52 | Phospholipid-Transporting ATPase |
| | | | | IH-like |
| 20a | CG105463-01 | 53 | 54 | Meningioma-Expressed Antigen |
| | | | | 6/11 (MEA6) (MEA11)-like |
| 20b | CG105463-02 | 55 | 56 | Meningioma-Expressed Antigen |
| | | | | 6/11 (MEA6) (MEA11)-like |
| 21a | CG105491-01 | 57 | 58 | Serine Protease-like |
| 22a | CG105954-01 | 59 | 60 | Neurofascin Precursor-like |
| 23a | CG105963-01 | 61 | 62 | Cadherin-like |
| 24a | CG105973-01 | 63 | 64 | Integrin Alpha 8-like |
| 24b | CG105973-02 | 65 | 66 | Integrin Alpha 8-like |
| 25a | CG106915-01 | 67 | 68 | Nogo Receptor Isoform-1-like |
| 26a | CG106924-01 | 69 | 70 | Nogo Receptor Isoform-2-like |
| 26b | 210062144 | 71 | 72 | Nogo Receptor Isoform-2-like |
| 27a | CG106942-01 | 73 | 74 | NRAMP-like Membrane Protein |
| 28a | CG107513-01 | 75 | 76 | Syntaxin Domain Containing |
| | | | | Protein-like |
| 29a | CG107533-02 | 77 | 78 | Tumor Necrosis Factor-like |
| 30a | CG107562-01 | 79 | 80 | Leucine-Rich Repeat Type III |
| | | | | Transmembrane-like |
| 30b | CG107562-02 | 81 | 82 | Leucine-Rich Repeat Type III |
| | | | | Transmembrane-like |
| 30c | 210086373 | 83 | 84 | Leucine-Rich Repeat Type III |
| | | | | Transmembrane-like |
| 30d | 210086403 | 85 | 86 | Leucine-Rich Repeat Type III |
| | | | | Transmembrane-like |
| 30e | 210086422 | 87 | 88 | Leucine-Rich Repeat Type III |
| | | | | Transmembrane-like |
| 31a | CG108184-01 | 89 | 90 | Transmembrane Protein Tm7-like |
| 31b | CG108184-02 | 91 | 92 | Transmembrane Protein Tm7-like |
| 31c | CG108184-03 | 93 | 94 | Transmembrane Protein Tm7-like |
| 32a | CG108238-01 | 95 | 96 | Sialic Acid Binding |
| | | | | Immunoglobulin-like |
| 33a | CG108695-01 | 97 | 98 | OB binding protein (SIGLEC)-like |
| 34a | CG109505-01 | 99 | 100 | Aldehyde Dehydrogenase-like |
| 35a | CG109742-01 | 101 | 102 | Latent Transforming Growth Factor |
| | | | | Beta Binding Protein 3-like |
| 35b | 207639410 | 103 | 104 | Latent Transforming Growth Factor |
| | | | | Beta Binding Protein 3-like |
| 35c | 207639427 | 105 | 106 | Latent Transforming Growth Factor |
| | | | | Beta Binding Protein 3-like |
| 35d | 207639438 | 107 | 108 | Latent Transforming Growth Factor |
| | | | | Beta Binding Protein 3-like |
| 35e | 207639448 | 109 | 110 | Latent Transforming Growth Factor |
| | | | | Beta Binding Protein 3-like |
| 36a | CG109844-01 | 111 | 112 | C4B-Binding Protein-like |
| 37a | CG110014-02 | 113 | 114 | Colon Carcinoma kinase 4-like |
| 37b | CG110014-03 | 115 | 116 | Colon Carcinoma kinase 4-like |
| 37c | CG110014-04 | 117 | 118 | Colon Carcinoma kinase 4-like |
| 38a | CG110187-01 | 119 | 120 | Alpha C1-like Protocadherin |
| 38b | CG110187-03 | 121 | 122 | Alpha C1-like Protocadherin |
| 39a | CG110205-01 | 123 | 124 | Disintegrin-like/Metalloprotease |
| | | | | (Reprolysin Type) with |
| | | | | Thrombospondin Type I Motif-like |
| 39b | CG110205-02 | 125 | 126 | Disintegrin-like/Metalloprotease |
| | | | | (Reprolysin Type) with |
| | | | | Thrombospondin Type I Motif-like |
| 39c | 207756942 | 127 | 128 | Disintegrin-like/Metalloprotease |
| | | | | (Reprolysin Type) with |
| | | | | Thrombospondin Type I Motif-like |
| 39d | 207756946 | 129 | 130 | Disintegrin-like/Metalloprotease |
| | | | | (Reprolysin Type) with |
| | | | | Thrombospondin Type I Motif-like |
| 39e | 207756950 | 131 | 132 | Disintegrin-like/Metalloprotease |
| | | | | (Reprolysin Type) with |
| | | | | Thrombospondin Type I Motif-like |
| 39f | 207756966 | 133 | 134 | Disintegrin-like/Metalloprotease |
| | | | | (Reprolysin Type) with |
| | | | | Thrombospondin Type I Motif-like |
| 40a | CG110242-01 | 135 | 136 | Ebnerin-like |
| 40b | 207728344 | 137 | 138 | Ebnerin-like |
| 40c | 207728348 | 139 | 140 | Ebnerin-like |
| 40d | 207728354 | 141 | 142 | Ebnerin-like |
| 40e | 207728365 | 143 | 144 | Ebnerin-like |
| 41a | CG99598-01 | 145 | 146 | Endosomal Glycoprotein |
| | | | | Precursor-like |

Table 1 indicates the homology of NOVX polypeptides to known protein families. Thus, the nucleic acids and polypeptides, antibodies and related compounds according to the invention corresponding to a NOVX as identified in column 1 of Table 1 will be useful in therapeutic and diagnostic applications implicated in, for example, pathologies and disorders associated with the known protein families identified in column 5 of Table 1.

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

Consistent with other known members of the family of proteins, identified in column 5 of Table 1, the NOVX polypeptides of the present invention show homology to, and contain domains that are characteristic of, other members of such protein families. Details of the sequence relatedness and domain analysis for each NOVX are presented in Example A.

The NOVX nucleic acids and polypeptides can also be used to screen for molecules, which inhibit or enhance NOVX activity or function. Specifically, the nucleic acids and polypeptides according to the invention may be used as targets for the identification of small molecules that modulate or inhibit diseases associated with the protein families listed in Table 1.

The NOVX nucleic acids and polypeptides are also useful for detecting specific cell types. Details of the expression analysis for each NOVX are presented in Example C. Accordingly, the NOVX nucleic acids, polypeptides, antibodies and related compounds according to the invention will have diagnostic and therapeutic applications in the detection of a variety of diseases with differential expression in normal vs. diseased tissues, e.g. detection of a variety of cancers.

Additional utilities for NOVX nucleic acids and polypeptides according to the invention are disclosed herein.

NOVX Clones

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

The NOVX genes and their corresponding encoded proteins are useful for preventing, treating or ameliorating medical conditions, e.g., by protein or gene therapy. Pathological conditions can be diagnosed by determining the amount of the new protein in a sample or by determining the presence of mutations in the new genes. Specific uses are described for each of the NOVX genes, based on the tissues in which they are most highly expressed. Uses include developing products -for the diagnosis or treatment of a variety of diseases and disorders.

The NOVX nucleic acids and proteins of the invention are useful in potential diagnostic and therapeutic applications and as research tools. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) a biological defense weapon.

In one specific embodiment, the invention includes an isolated polypeptide comprising an amino acid sequence selected from the group consisting of: (a) a mature form of the amino acid sequence selected from the group consisting of SEQ ID NO: 2n, wherein n is an integer between 1 and 73; (b) a variant of a mature form of the amino acid sequence selected from the group consisting of SEQ ID NO: 2n, wherein n is an integer between 1 and 73, wherein any amino acid in the mature form is changed to a different amino acid, provided that no more than 15% of the amino acid residues in the sequence of the mature form are so changed; (c) an amino acid sequence selected from the group consisting of SEQ ID NO: 2n, wherein n is an integer between 1 and 73; (d) a variant of the amino acid sequence selected from the group consisting of SEQ ID NO:2n, wherein n is an integer between 1 and 73 wherein any amino acid specified in the chosen sequence is changed to a different amino acid, provided that no more than 15% of the amino acid residues in the sequence are so changed; and (e) a fragment of any of (a) through (d).

In another specific embodiment, the invention includes an isolated nucleic acid molecule comprising a nucleic acid sequence encoding a polypeptide comprising an amino acid sequence selected from the group consisting of: (a) a mature form of the amino acid sequence given SEQ ID NO: 2n, wherein n is an integer between 1 and 73; (b) a variant of a mature form of the amino acid sequence selected from the group consisting of SEQ ID NO: 2n, wherein n is an integer between 1 and 73 wherein any amino acid in the mature form of the chosen sequence is changed to a different amino acid, provided that no more than 15% of the amino acid residues in the sequence of the mature form are so changed; (c) the amino acid sequence selected from the group consisting of SEQ ID NO: 2n, wherein n is an integer between 1 and 73; (d) a variant of the amino acid sequence selected from the group consisting of SEQ ID NO: 2n, wherein n is an integer between 1 and 73, in which any amino acid specified in the chosen sequence is changed to a different amino acid, provided that no more than 15% of the amino acid residues in the sequence are so changed; (e) a nucleic acid fragment encoding at least a portion of a polypeptide comprising the amino acid sequence selected from the group consisting of SEQ ID NO: 2n, wherein n is an integer between 1 and 73 or any variant of said polypeptide wherein any amino acid of the chosen sequence is changed to a different amino acid, provided that no more than 10% of the amino acid residues in the sequence are so changed; and (f) the complement of any of said nucleic acid molecules.

In yet another specific embodiment, the invention includes an isolated nucleic acid molecule, wherein said nucleic acid molecule comprises a nucleotide sequence selected from the group consisting of: (a) the nucleotide sequence selected from the group consisting of SEQ ID NO: 2n−1, wherein n is an integer between 1 and 73; (b) a nucleotide sequence wherein one or more nucleotides in the nucleotide sequence selected from the group consisting of SEQ ID NO: 2n−1, wherein n is an integer between 1 and 73 is changed from that selected from the group consisting of the chosen sequence to a different nucleotide provided that no more than 15% of the nucleotides are so changed; (c) a nucleic acid fragment of the sequence selected from the group consisting of SEQ ID NO: 2n−1, wherein n is an integer between 1 and 73; and (d) a nucleic acid fragment wherein one or more nucleotides in the nucleotide sequence selected from the group consisting of SEQ ID NO: 2n−1, wherein n is an integer between 1 and 73 is changed from that selected from the group consisting of the chosen sequence to a different nucleotide provided that no more than 15% of the nucleotides are so changed.

NOVX Nucleic Acids and Polypeptides

One aspect of the invention pertains to isolated nucleic acid molecules that encode NOVX polypeptides or biologically active portions thereof. Also included in the invention are nucleic acid fragments sufficient for use as hybridization probes to identify NOVX-encoding nucleic acids (e.g., NOVX mRNA's) and fragments for use as PCR primers for the amplification and/or mutation of NOVX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

A NOVX nucleic acid can encode a mature NOVX polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full-length gene product encoded by the corresponding gene. Alternatively, it may be defined as the polypeptide, precursor or proprotein encoded by an ORF described herein. The product “mature” form arises, again by way of nonlimiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristylation or phosphorylation. In general, a mature polypeptide or protein may result from the operation of only one of these processes, or a combination of any of them.

The term “probes”, as utilized herein, refers to nucleic acid sequences of variable length, preferably between at least about 10 nucleotides (nt), 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single- or double-stranded and designed to have specificity in PCR, membrane-based hybridization technologies, or ELISA-like technologies.

The term “isolated” nucleic acid molecule, as utilized herein, is one, which is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated NOVX nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the invention, e.g., a nucleic acid molecule having the nucleotide sequence of SEQ ID NO:2n−1, wherein n is an integer between 1-73, or a complement of this aforementioned nucleotide sequence, can be isolated using standard molecular biology techniques and the sequence information provided herein. Using all or a portion of the nucleic acid sequence of SEQ ID NO:2n−1, wherein n is an integer between 1-73, as a hybridization probe, NOVX molecules can be isolated using standard hybridization and cloning techniques (e.g., as described in Sambrook, et al., (eds.), Molecular Cloning: A Laboratory Manual 2^{nd} Ed., Cold Spring Harbor Laboratory Press, Cold Spring Harbor, N.Y., 1989; and Ausubel, et al., (eds.), Current Protocols in Molecular Biology, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to NOVX nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides of SEQ ID NO:2n−1, wherein n is an integer between 1-73, or a complement thereof. Oligonucleotides may be chemically synthesized and may also be used as probes.

In another embodiment, an isolated nucleic acid molecule of the invention comprises a nucleic acid molecule that is a complement of the nucleotide sequence SEQ ID NO:2n−1, wherein n is an integer between 1-73, or a portion of this nucleotide sequence (e.g., a fragment that can be used as a probe or primer or a fragment encoding a biologically-active portion of a NOVX polypeptide). A nucleic acid molecule that is complementary to the nucleotide sequence of SEQ ID NO:2n−1, wherein n is an integer between 1-73, is one that is sufficiently complementary to the nucleotide sequence of SEQ ID NO:2n−1, wherein n is an integer between 1-73, that it can hydrogen bond with little or no mismatches to the nucleotide sequence of SEQ ID NO:2n−1, wherein n is an integer between 1-73, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type. Homologs are nucleic acid sequences or amino acid sequences of a particular gene that are derived from different species.

A full-length NOVX clone is identified as containing an ATG translation start codon and an in-frame stop codon. Any disclosed NOVX nucleotide sequence lacking an ATG start codon therefore encodes a truncated C-terminal fragment of the respective NOVX polypeptide, and requires that the corresponding full-length cDNA extend in the 5′ direction of the disclosed sequence. Any disclosed NOVX nucleotide sequence lacking an in-frame stop codon similarly encodes a truncated N-terminal fragment of the respective NOVX polypeptide, and requires that the corresponding full-length cDNA extend in the 3′ direction of the disclosed sequence.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., Current Protocols in Molecular Biology, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of NOVX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for a NOVX polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human NOVX protein. Homologous nucleic acid sequences include those nucleic acid sequences that encode conservative amino acid substitutions (see below) in SEQ ID NO:2n−1, wherein n is an integer between 1-73, as well as a polypeptide possessing NOVX biological activity. Various biological activities of the NOVX proteins are described below.

A NOVX polypeptide is encoded by the open reading frame (“ORF”) of a NOVX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bonafide cellular protein, a minimum size requirement is often set, e.g., a stretch of DNA that would encode a protein of 50 amino acids or more.

The nucleotide sequences determined from the cloning of the human NOVX genes allows for the generation of probes and primers designed for use in identifying and/or cloning NOVX homologues in other cell types, e.g. from other tissues, as well as NOVX homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence of SEQ ID NO:2n−1, wherein n is an integer between 1-73; or an anti-sense strand nucleotide sequence of SEQ ID NO:2n−1, wherein n is an integer between 1-73; or of a naturally occurring mutant of SEQ ID NO:2n−1, wherein n is an integer between 1-73.

Probes based on the human NOVX nucleotide sequences can be used to detect transcripts or genomic sequences encoding the same or homologous proteins. In various embodiments, the probe further comprises a label group attached thereto, e.g. the label group can be a radioisotope, a fluorescent compound, an enzyme, or an enzyme co-factor. Such probes can be used as a part of a diagnostic test kit for identifying cells or tissues which mis-express a NOVX protein, such as by measuring a level of a NOVX-encoding nucleic acid in a sample of cells from a subject e.g., detecting NOVX mRNA levels or determining whether a genomic NOVX gene has been mutated or deleted.

“A polypeptide having a biologically-active portion of a NOVX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of NOVX” can be prepared by isolating a portion of SEQ ID NO:2n−1, wherein n is an integer between 1-73, that encodes a polypeptide having a NOVX biological activity (the biological activities of the NOVX proteins are described below), expressing the encoded portion of NOVX protein (e.g., by recombinant expression in vitro) and assessing the activity of the encoded portion of NOVX.

NOVX Nucleic Acid and Polypeptide Variants

The invention further encompasses nucleic acid molecules that differ from the nucleotide sequences of SEQ ID NO:2n−1, wherein n is an integer between 1-73, due to degeneracy of the genetic code and thus encode the same NOVX proteins as that encoded by the nucleotide sequences of SEQ ID NO:2n−1, wherein n is an integer between 1-73. In another embodiment, an isolated nucleic acid molecule of the invention has a nucleotide sequence encoding a protein having an amino acid sequence of SEQ ID NO:2n, wherein n is an integer between 1-73.

In addition to the human NOVX nucleotide sequences of SEQ ID NO:2n−1, wherein n is an integer between 1-73, it will be appreciated by those skilled in the art that DNA sequence polymorphisms that lead to changes in the amino acid sequences of the NOVX polypeptides may exist within a population (e.g., the human population). Such genetic polymorphism in the NOVX genes may exist among individuals within a population due to natural allelic variation. As used herein, the terms “gene” and “recombinant gene” refer to nucleic acid molecules comprising an open reading frame (ORF) encoding a NOVX protein, preferably a vertebrate NOVX protein. Such natural allelic variations can typically result in 1-5% variance in the nucleotide sequence of the NOVX genes. Any and all such nucleotide variations and resulting amino acid polymorphisms in the NOVX polypeptides, which are the result of natural allelic variation and that do not alter the functional activity of the NOVX polypeptides, are intended to be within the scope of the invention.

Moreover, nucleic acid molecules encoding NOVX proteins from other species, and thus that have a nucleotide sequence that differs from any one of the human SEQ ID NO:2n−1, wherein n is an integer between 1-73, are intended to be within the scope of the invention. Nucleic acid molecules corresponding to natural allelic variants and homologues of the NOVX cDNAs of the invention can be isolated based on their homology to the human NOVX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NO:2n−1, wherein n is an integer between 1-73. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding NOVX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), Current Protocols in Molecular Biology, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. An isolated nucleic acid molecule of the invention that hybridizes under stringent conditions to any one of the sequences of SEQ ID NO:2n−1, wherein n is an integer between 1-73, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NO:2n−1, wherein n is an integer between 1-73, or fragments, analogs or derivatives thereof, under conditions of moderate stringency is provided. A non-limiting example of moderate stringency hybridization conditions are hybridization in 6×SSC, 5×Reinhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known within the art. See, e.g., Ausubel, et al. (eds.), 1993, Current Protocols in Molecular Biology, John Wiley & Sons, NY, and Krieger, 1990; Gene Transfer and Expression, A Laboratory Manual, Stockton Press, NY.

In a third embodiment, a nucleic acid that is hybridizable to the nucleic acid molecule comprising the nucleotide sequences of SEQ ID NO:2n−1, wherein n is an integer between 1-73, or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/volt) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, Current Protocols in Molecular Biology, John Wiley & Sons, NY, and Kriegler, 1990, Gene Transfer and Expression, A Laboratory Manual, Stockton Press, NY; Shilo and Weinberg, 1981. Proc Natl Acad Sci USA 78:6789-6792.

Conservative Mutations

In addition to naturally-occurring allelic variants of NOVX sequences that may exist in the population, the skilled artisan will further appreciate that changes can be introduced by mutation into the nucleotide sequences of SEQ ID NO:2n−1, wherein n is an integer between 1-73, thereby leading to changes in the amino acid sequences of the encoded NOVX proteins, without altering the functional ability of said NOVX proteins. For example, nucleotide substitutions leading to amino acid substitutions at “non-essential” amino acid residues can be made in the sequence of SEQ ID NO:2n, wherein n is an integer between 1-73. A “non-essential” amino acid residue is a residue that can be altered from the wild-type sequences of the NOVX proteins without altering their biological activity, whereas an “essential” amino acid residue is required for such biological activity. For example, amino acid residues that are conserved among the NOVX proteins of the invention are particularly non-amenable to alteration. Amino acids for which conservative substitutions can be made are well-known within the art.

Another aspect of the invention pertains to nucleic acid molecules encoding NOVX proteins that contain changes in amino acid residues that are not essential for activity. Such NOVX proteins differ in amino acid sequence from any one of SEQ ID NO:2n−1, wherein n is an integer between 1-73, yet retain biological activity. In one embodiment, the isolated nucleic acid molecule comprises a nucleotide sequence encoding a protein, wherein the protein comprises an amino acid sequence at least about 45% homologous to the amino acid sequences of SEQ ID NO:2n, wherein n is an integer between 1-73. Preferably, the protein encoded by the nucleic acid molecule is at least about 60% homologous to SEQ ID NO:2n, wherein n is an integer between 1-73; more preferably at least about 70% homologous to SEQ ID NO:2n, wherein n is an integer between 1-73; still more preferably at least about 80% homologous to SEQ ID NO:2n, wherein n is an integer between 1-73; even more preferably at least about 90% homologous to SEQ ID NO:2n, wherein n is an integer between 1-73; and most preferably at least about 95% homologous to SEQ ID NO:2n, wherein n is an integer between 1-73.

An isolated nucleic acid molecule encoding a NOVX protein homologous to the protein of SEQ ID NO:2n, wherein n is an integer between 1-73, can be created by introducing one or more nucleotide substitutions, additions or deletions into the nucleotide sequence of SEQ ID NO:2n−1, wherein n is an integer between 1-73, such that one or more amino acid substitutions, additions or deletions are introduced into the encoded protein.

Mutations can be introduced into any of SEQ ID NO:2n−1, wherein n is an integer between 1-73, by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the NOVX protein is replaced with another amino acid residue from the same side chain family. Alternatively, in another embodiment, mutations can be introduced randomly along all or part of a NOVX coding sequence, such as by saturation mutagenesis, and the resultant mutants can be screened for NOVX biological activity to identify mutants that retain activity. Following mutagenesis of any one of SEQ ID NO:2n−1, wherein n is an integer between 1-73, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, HFY, wherein the letters within each group represent the single letter amino acid code.

In one embodiment, a mutant NOVX protein can be assayed for (i) the ability to form protein:protein interactions with other NOVX proteins, other cell-surface proteins, or biologically-active portions thereof, (ii) complex formation between a mutant NOVX protein and a NOVX ligand; or (iii) the ability of a mutant NOVX protein to bind to an intracellular target protein or biologically-active portion thereof; (e.g. avidin proteins).

In yet another embodiment, a mutant NOVX protein can be assayed for the ability to regulate a specific biological function (e.g., regulation of insulin release).

Antisense Nucleic Acids

Another aspect of the invention pertains to isolated antisense nucleic acid molecules that are hybridizable to or complementary to the nucleic acid molecule comprising the nucleotide sequence of SEQ ID NO:2n−1, wherein n is an integer between 1-73, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire NOVX coding strand, or to only a portion thereof. Nucleic acid molecules encoding fragments, homologs, derivatives and analogs of a NOVX protein of SEQ ID NO:2n, wherein n is an integer between 1-73, or antisense nucleic acids complementary to a NOVX nucleic acid sequence of SEQ ID NO:2n−1, wherein n is an integer between 1-73, are additionally provided.

In one embodiment, an antisense nucleic acid molecule is antisense to a “coding region” of the coding strand of a nucleotide sequence encoding a NOVX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the NOVX protein. The term “noncoding region” refers to 5′ and 3′ sequences which flank the coding region that are not translated into amino acids (i.e., also referred to as 5′ and 3′ untranslated regions).

Given the coding strand sequences encoding the NOVX protein disclosed herein, antisense nucleic acids of the invention can be designed according to the rules of Watson and Crick or Hoogsteen base pairing. The antisense nucleic acid molecule can be complementary to the entire coding region of NOVX mRNA, but more preferably is an oligonucleotide that is antisense to only a portion of the coding or noncoding region of NOVX mRNA. For example, the antisense oligonucleotide can be complementary to the region surrounding the translation start site of NOVX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a NOVX protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other. See, e.g., Gaultier, et al., 1987. Nucl. Acids Res. 15: 6625-6641. The antisense nucleic acid molecule can also comprise a 2′-o-methylribonucleotide (See, e.g., Inoue, et al. 1987. Nucl Acids Res. 15: 6131-6148) or a chimeric RNA-DNA analogue (See, e.g., Inoue, et al., 1987. FEBS Lett. 215: 327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave NOVX mRNA transcripts to thereby inhibit translation of NOVX mRNA. A ribozyme having specificity for a NOVX-encoding nucleic acid can be designed based upon the nucleotide sequence of a NOVX cDNA disclosed herein (i.e., any one of SEQ ID NO:2n−1, wherein n is an integer between 1-73). For example, a derivative of a Tetrahymena L-19 IVS RNA can be constructed in which the nucleotide sequence of the active site is complementary to the nucleotide sequence to be cleaved in a NOVX-encoding mRNA. See, e.g., U.S. Pat. No. 4,987,071 to Cech, et al. and U.S. Pat. No. 5,116,742 to Cech, et al. NOVX mRNA can also be used to select a catalytic RNA having a specific ribonuclease activity from a pool of RNA molecules. See, e.g., Bartel et al., (1993) Science 261:1411-1418.

Alternatively, NOVX gene expression can be inhibited by targeting nucleotide sequences complementary to the regulatory region of the NOVX nucleic acid (e.g., the NOVX promoter and/or enhancers) to form triple helical structures that prevent transcription of the NOVX gene in target cells. See, e.g., Helene, 1991. Anticancer Drug Des. 6: 569-84; Helene, et al. 1992. Ann. N.Y. Acad. Sci. 660: 27-36; Maher, 1992. Bioassays 14: 807-15.

In various embodiments, the NOVX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAS” refer to nucleic acid mimics (e.g., DNA mimics) in which the deoxyribose phosphate backbone is replaced by a pseudopeptide backbone and only the four natural nucleotide bases are retained. The neutral backbone of PNAs has been shown to allow for specific hybridization to DNA and RNA under conditions of low ionic strength. The synthesis of PNA oligomer can be performed using standard solid phase peptide synthesis protocols as described in Hyrup, et al., 1996. supra; Perry-O'Keefe, et al., 1996. Proc. Natl. Acad. Sci. USA 93: 14670-14675.

PNAs of NOVX can be used in therapeutic and diagnostic applications. For example, PNAs can be used as antisense or antigene agents for sequence-specific modulation of gene expression by, e.g., inducing transcription or translation arrest or inhibiting replication. PNAs of NOVX can also be used, for example, in the analysis of single base pair mutations in a gene (e.g., PNA directed PCR clamping; as artificial restriction enzymes when used in combination with other enzymes, e.g., S₁ nucleases (See, Hyrup, et al., 1996.supra); or as probes or primers for DNA sequence and hybridization (See, Hyrup, et al., 1996, supra; Perry-O'Keefe, et al., 1996. supra).

In another embodiment, PNAs of NOVX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of NOVX can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleotide bases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

NOVX Polypeptides

A polypeptide according to the invention includes a polypeptide including the amino acid sequence of NOVX polypeptides whose sequences are provided in any one of SEQ ID NO:2n, wherein n is an integer between 1-73. The invention also includes a mutant or variant protein any of whose residues may be changed from the corresponding residues shown in any one of SEQ ID NO:2n, wherein n is an integer between 1-73, while still encoding a protein that maintains its NOVX activities and physiological functions, or a functional fragment thereof.

In general, a NOVX variant that preserves NOVX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated NOVX proteins, and biologically-active portions thereof, or derivatives, fragments, analogs or homologs thereof. Also provided are polypeptide fragments suitable for use as immunogens to raise anti-NOVX antibodies. In one embodiment, native NOVX proteins can be isolated from cells or tissue sources by an appropriate purification scheme using standard protein purification techniques. In another embodiment, NOVX proteins are produced by recombinant DNA techniques. Alternative to recombinant expression, a NOVX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the NOVX protein is derived, or substantially free from chemical precursors or other chemicals when chemically synthesized. The language “substantially free of cellular material” includes preparations of NOVX proteins in which the protein is separated from cellular components of the cells from which it is isolated or recombinantly-produced. In one embodiment, the language “substantially free of cellular material” includes preparations of NOVX proteins having less than about 30% (by dry weight) of non-NOVX proteins (also referred to herein as a “contaminating protein”), more preferably less than about 20% of non-NOVX proteins, still more preferably less than about 10% of non-NOVX proteins, and most preferably less than about 5% of non-NOVX proteins. When the NOVX protein or biologically-active portion thereof is recombinantly-produced, it is also preferably substantially free of culture medium, i.e., culture medium represents less than about 20%, more preferably less than about 10%, and most preferably less than about 5% of the volume of the NOVX protein preparation.

The language “substantially free of chemical precursors or other chemicals” includes preparations of NOVX proteins in which the protein is separated from chemical precursors or other chemicals that are involved in the synthesis of the protein. In one embodiment, the language “substantially free of chemical precursors or other chemicals” includes preparations of NOVX proteins having less than about 30% (by dry weight) of chemical precursors or non-NOVX chemicals, more preferably less than about 20% chemical precursors or non-NOVX chemicals, still more preferably less than about 10% chemical precursors or non-NOVX chemicals, and most preferably less than about 5% chemical precursors or non-NOVX chemicals.

Biologically-active portions of NOVX proteins include peptides comprising amino acid sequences sufficiently homologous to or derived from the amino acid sequences of the NOVX proteins (e.g., the amino acid sequence of SEQ ID NO:2n, wherein n is an integer between 1-73) that include fewer amino acids than the full-length NOVX proteins, and exhibit at least one activity of a NOVX protein. Typically, biologically-active portions comprise a domain or motif with at least one activity of the NOVX protein. A biologically-active portion of a NOVX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native NOVX protein.

In an embodiment, the NOVX protein has an amino acid sequence of SEQ ID NO:2n, wherein n is an integer between 1-73. In other embodiments, the NOVX protein is substantially homologous to SEQ ID NO:2n, wherein n is an integer between 1-73, and retains the functional activity of the protein of SEQ ID NO:2n, wherein n is an integer between 1-73, yet differs in amino acid sequence due to natural allelic variation or mutagenesis, as described in detail, below. Accordingly, in another embodiment, the NOVX protein is a protein that comprises an amino acid sequence at least about 45% homologous to the amino acid sequence of SEQ ID NO:2n, wherein n is an integer between 1-73, and retains the functional activity of the NOVX proteins of SEQ ID NO:2n, wherein n is an integer between 1-73.

Determining Homology between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence of SEQ ID NO:2n−1, wherein n is an integer between 1-73.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides NOVX chimeric or fusion proteins. As used herein, a NOVX “chimeric protein” or “fusion protein” comprises a NOVX polypeptide operatively-linked to a non-NOVX polypeptide. An “NOVX polypeptide” refers to a polypeptide having an amino acid sequence corresponding to a NOVX protein of SEQ ID NO:2n, wherein n is an integer between 1-73, whereas a “non-NOVX polypeptide” refers to a polypeptide having an amino acid sequence corresponding to a protein that is not substantially homologous to the NOVX protein, e.g., a protein that is different from the NOVX protein and that is derived from the same or a different organism. Within a NOVX fusion protein the NOVX polypeptide can correspond to all or a portion of a NOVX protein. In one embodiment, a NOVX fusion protein comprises at least one biologically-active portion of a NOVX protein. In another embodiment, a NOVX fusion protein comprises at least two biologically-active portions of a NOVX protein. In yet another embodiment, a NOVX fusion protein comprises at least three biologically-active portions of a NOVX protein. Within the fusion protein, the term “operatively-linked” is intended to indicate that the NOVX polypeptide and the non-NOVX polypeptide are fused in-frame with one another. The non-NOVX polypeptide can be fused to the N-terminus or C-terminus of the NOVX polypeptide.

In one embodiment, the fusion protein is a GST-NOVX fusion protein in which the NOVX sequences are fused to the C-terminus of the GST (glutathione S-transferase) sequences. Such fusion proteins can facilitate the purification of recombinant NOVX polypeptides.

In another embodiment, the fusion protein is a NOVX protein containing a heterologous signal sequence at its N-terminus. In certain host cells (e.g., mammalian host cells), expression and/or secretion of NOVX can be increased through use of a heterologous signal sequence.

In yet another embodiment, the fusion protein is a NOVX-immunoglobulin fusion protein in which the NOVX sequences are fused to sequences derived from a member of the immunoglobulin protein family. The NOVX-immunoglobulin fusion proteins of the invention can be incorporated into pharmaceutical compositions and administered to a subject to inhibit an interaction between a NOVX ligand and a NOVX protein on the surface of a cell, to thereby suppress NOVX-mediated signal transduction in vivo. The NOVX-immunoglobulin fusion proteins can be used to affect the bioavailability of a NOVX cognate ligand. Inhibition of the NOVX ligand/NOVX interaction may be useful therapeutically for both the treatment of proliferative and differentiative disorders, as well as modulating (e.g. promoting or inhibiting) cell survival. Moreover, the NOVX-immunoglobulin fusion proteins of the invention can be used as immunogens to produce anti-NOVX antibodies in a subject, to purify NOVX ligands, and in screening assays to identify molecules that inhibit the interaction of NOVX with a NOVX ligand.

A NOVX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) Current Protocols in Molecular Biology, John Wiley & Sons, 1992). Moreover, many expression vectors are commercially available that already encode a fusion moiety (e.g., a GST polypeptide). A NOVX-encoding nucleic acid can be cloned into such an expression vector such that the fusion moiety is linked in-frame to the NOVX protein.

NOVX Agonists and Antagonists

The invention also pertains to variants of the NOVX proteins that function as either NOVX agonists (i.e., mimetics) or as NOVX antagonists. Variants of the NOVX protein can be generated by mutagenesis (e.g., discrete point mutation or truncation of the NOVX protein). An agonist of the NOVX protein can retain substantially the same, or a subset of, the biological activities of the naturally occurring form of the NOVX protein. An antagonist of the NOVX protein can inhibit one or more of the activities of the naturally occurring form of the NOVX protein by, for example, competitively binding to a downstream or upstream member of a cellular signaling cascade which includes the NOVX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the NOVX proteins.

Variants of the NOVX proteins that function as either NOVX agonists (i.e., mimetics) or as NOVX antagonists can be identified by screening combinatorial libraries of mutants (e.g., truncation mutants) of the NOVX proteins for NOVX protein agonist or antagonist activity. In one embodiment, a variegated library of NOVX variants is generated by combinatorial mutagenesis at the nucleic acid level and is encoded by a variegated gene library. A variegated library of NOVX variants can be produced by, for example, enzymatically ligating a mixture of synthetic oligonucleotides into gene sequences such that a degenerate set of potential NOVX sequences is expressible as individual polypeptides, or alternatively, as a set of larger fusion proteins (e.g., for phage display) containing the set of NOVX sequences therein. There are a variety of methods which can be used to produce libraries of potential NOVX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential NOVX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198:1056; Ike, et al., 1983. Nucl. Acids Res. 11:477.

Polypeptide Libraries

In addition, libraries of fragments of the NOVX protein coding sequences can be used to generate a variegated population of NOVX fragments for screening and subsequent selection of variants of a NOVX protein. In one embodiment, a library of coding sequence fragments can be generated by treating a double stranded PCR fragment of a NOVX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with S₁ nuclease, and ligating the resulting fragment library into an expression vector. By this method, expression libraries can be derived which encodes N-terminal and internal fragments of various sizes of the NOVX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of NOVX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify NOVX variants. See, e.g., Arkin and Yourvan, 1992. Proc. Natl. Acad. Sci. USA 89: 7811-7815; Delgrave, et al., 1993. Protein Engineering 6:327-331.

NOVX Antibodies

The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab′} and F_{(ab′)2} fragments, and an F_{ab} expression library. In general, antibody molecules obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated protein of the invention intended to serve as an antigen, or a portion or fragment thereof, can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein, such as an amino acid sequence of SEQ ID NO:2n, wherein n is an integer between 1-73, and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of NOVX that is located on the surface of the protein, e.g., a hydrophilic region. A hydrophobicity analysis of the human NOVX protein sequence will indicate which regions of a NOVX polypeptide are particularly hydrophilic and, therefore, encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each incorporated herein by reference in their entirety. Antibodies that are specific for one or more domains within an antigenic protein, or derivatives, fragments, analogs or homologs thereof, are also provided herein.

The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. A NOVX polyppeptide or a fragment thereof comprises at least one antigenic epitope. An anti-NOVX antibody of the present invention is said to specifically bind to antigen NOVX when the equilibrium binding constant (K_{D}) is ≦1 μM, preferably ≦100 nM, more preferably ≦10 nM, and most preferably ≦100 pM to about 1 pM, as measured by assays such as radioligand binding assays or similar assays known to those skilled in the art.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow E, and Lane D, 1988, Cold Spring Harbor Laboratory Press, Cold Spring Harbor, N.Y., incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). It is an objective, especially important in therapeutic applications of monoclonal antibodies, to identify antibodies having a high degree of specificity and a high binding affinity for the target antigen.

After the desired hybridoma cells are identified, the clones can be subcloned by limiting dilution procedures and grown by standard methods (Goding,1986). Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subdlones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

Human Antibodies

Fully human antibodies essentially relate to antibody molecules in which the entire sequence of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: Monoclonal Antibodies and Cancer Therapy, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: Monoclonal Antibodies and Cancer Therapy, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368, 812-13 (1994)); Fishwild et al, (Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published May 13, 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fc regions and can thereby have enhanced complement lysis and ADCC capabilities. See Stevenson et al., Anti-Cancer Drug Design, 3: 219-230 (1989).

Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol)propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl)hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

Immunoliposomes

The antibodies disclosed herein can also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem., 257: 286-288 (1982) via a disulfide-interchange reaction. A chemotherapeutic agent (such as Doxorubicin) is optionally contained within the liposome. See Gabizon et al., J. National Cancer Inst., 81(19): 1484 (1989).

Diagnostic Applications of Antibodies Directed against the Proteins of the Invention

Antibodies directed against a protein of the invention may be used in methods known within the art relating to the localization and/or quantitation of the protein (e.g., for use in measuring levels of the protein within appropriate physiological samples, for use in diagnostic methods, for use in imaging the protein, and the like). In a given embodiment, antibodies against the proteins, or derivatives, fragments, analogs or homologs thereof, that contain the antigen binding domain, are utilized as pharmacologically-active compounds (see below).

An antibody specific for a protein of the invention can be used to isolate the protein by standard techniques, such as immunoaffinity chromatography or immunoprecipitation. Such an antibody can facilitate the purification of the natural protein antigen from cells and of recombinantly produced antigen expressed in host cells. Moreover, such an antibody can be used to detect the antigenic protein (e.g., in a cellular lysate or cell supernatant) in order to evaluate the abundance and pattern of expression of the antigenic protein. Antibodies directed against the protein can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Antibody Therapeutics

Antibodies of the invention, including polyclonal, monoclonal, humanized and fully human antibodies, may used as therapeutic agents. Such agents will generally be employed to treat or prevent a disease or pathology in a subject. An antibody preparation, preferably one having high specificity and high affinity for its target antigen, is administered to the subject and will generally have an effect due to its binding with the target. Such an effect may be one of two kinds, depending on the specific nature of the interaction between the given antibody molecule and the target antigen in question. In the first instance, administration of the antibody may abrogate or inhibit the binding of the target with an endogenous ligand to which it naturally binds. In this case, the antibody binds to the target and masks a binding site of the naturally occurring ligand, wherein the ligand serves as an effector molecule. Thus the receptor mediates a signal transduction pathway for which ligand is responsible.

Alternatively, the effect may be one in which the antibody elicits a physiological result by virtue of binding to an effector binding site on the target molecule. In this case the target, a receptor having an endogenous ligand which may be absent or defective in the disease or pathology, binds the antibody as a surrogate effector ligand, initiating a receptor-based signal transduction event by the receptor.

A therapeutically effective amount of an antibody of the invention relates generally to the amount needed to achieve a therapeutic objective. As noted above, this may be a binding interaction between the antibody and its target antigen that, in certain cases, interferes with the functioning of the target, and in other cases, promotes a physiological response. The amount required to be administered will furthermore depend on the binding affinity of the antibody for its specific antigen, and will also depend on the rate at which an administered antibody is depleted from the free volume other subject to which it is administered. Common ranges for therapeutically effective dosing of an antibody or antibody fragment of the invention may be, by way of nonlimiting example, from about 0.1 mg/kg body weight to about 50 mg/kg body weight. Common dosing frequencies may range, for example, from twice daily to once a week.

Pharmaceutical Compositions of Antibodies

Antibodies specifically binding a protein of the invention, as well as other molecules identified by the screening assays disclosed herein, can be administered for the treatment of various disorders in the form of pharmaceutical compositions. Principles and considerations involved in preparing such compositions, as well as guidance in the choice of components are provided, for example, in Remington: The Science And Practice Of Pharmacy 19th ed. (Alfonso R. Gennaro, et al., editors) Mack Pub. Co., Easton, Pa.: 1995; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York.

If the antigenic protein is intracellular and whole antibodies are used as inhibitors, internalizing antibodies are preferred. However, liposomes can also be used to deliver the antibody, or an antibody fragment, into cells. Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. See, e.g., Marasco et al., Proc. Natl. Acad. Sci. USA, 90: 7889-7893 (1993). The formulation herein can also contain more than one active compound as necessary for the particular indication being treated, preferably those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

ELISA Assay

An agent for detecting an analyte protein is an antibody capable of binding to an analyte protein, preferably an antibody with a detectable label. Antibodies can be polyclonal, or more preferably, monoclonal. An intact antibody, or a fragment thereof (e.g., F_{ab} or F_{(ab)2}) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. Included within the usage of the term “biological sample”, therefore, is blood and a fraction or component of blood including blood serum, blood plasma, or lymph. That is, the detection method of the invention can be used to detect an analyte mRNA, protein, or genomic DNA in a biological sample in vitro as well as in vivo. For example, in vitro techniques for detection of an analyte mRNA include Northern hybridizations and in situ hybridizations. In vitro techniques for detection of an analyte protein include enzyme linked immunosorbent assays (ELISAs), Western blots, immunoprecipitations, and immunofluorescence. In vitro techniques for detection of an analyte genomic DNA include Southern hybridizations. Procedures for conducting immunoassays are described, for example in “ELISA: Theory and Practice: Methods in Molecular Biology”, Vol. 42, J. R. Crowther (Ed.) Human Press, Totowa, N.J., 1995; “Immunoassay”, E. Diamandis and T. Christopoulus, Academic Press, Inc., San Diego, Calif., 1996; and “Practice and Thory of Enzyme Immunoassays”, P. Tijssen, Elsevier Science Publishers, Amsterdam, 1985. Furthermore, in vivo techniques for detection of an analyte protein include introducing into a subject a labeled anti-an analyte protein antibody. For example, the antibody can be labeled with a radioactive marker whose presence and location in a subject can be detected by standard imaging techniques.

NOVX Recombinant Expression Vectors and Host Cells

Another aspect of the invention pertains to vectors, preferably expression vectors, containing a nucleic acid encoding a NOVX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, Gene Expression Technology: Methods in Enzymology 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., NOVX proteins, mutant forms of NOVX proteins, fusion proteins, etc.).

The recombinant expression vectors of the invention can be designed for expression of NOVX proteins in prokaryotic or eukaryotic cells. For example, NOVX proteins can be expressed in bacterial cells such as Escherichia coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., Gene Expression Technology: Methods in Enzymology 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, Gene Expression Technology: Methods in Enzymology 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the NOVX expression vector is a yeast expression vector. Examples of vectors for expression in yeast Saccharomyces cerivisae include pYepSec1 (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, NOVX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., Molecular Cloning: A Laboratory Manual. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to NOVX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, NOVX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding NOVX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (ie., express) NOVX protein. Accordingly, the invention further provides methods for producing NOVX protein using the host cells of the invention. In one embodiment, the method comprises culturing the host cell of invention (into which a recombinant expression vector encoding NOVX protein has been introduced) in a suitable medium such that NOVX protein is produced. In another embodiment, the method further comprises isolating NOVX protein from the medium or the host cell.

Transgenic NOVX Animals

The host cells of the invention can also be used to produce non-human transgenic animals. For example, in one embodiment, a host cell of the invention is a fertilized oocyte or an embryonic stem cell into which NOVX protein-coding sequences have been introduced. Such host cells can then be used to create non-human transgenic animals in which exogenous NOVX sequences have been introduced into their genome or homologous recombinant animals in which endogenous NOVX sequences have been altered. Such animals are useful for studying the function and/or activity of NOVX protein and for identifying and/or evaluating modulators of NOVX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous NOVX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing NOVX-encoding nucleic acid into the male pronuclei of a fertilized oocyte (e.g., by microinjection, retroviral infection) and allowing the oocyte to develop in a pseudopregnant female foster animal. The human NOVX cDNA sequences, i.e., any one of SEQ ID NO:2n−1, wherein n is an integer between 1-73, can be introduced as a transgene into the genome of a non-human animal. Alternatively, a non-human homologue of the human NOVX gene, such as a mouse NOVX gene, can be isolated based on hybridization to the human NOVX cDNA (described further supra) and used as a transgene. Intronic sequences and polyadenylation signals can also be included in the transgene to increase the efficiency of expression of the transgene. A tissue-specific regulatory sequence(s) can be operably-linked to the NOVX transgene to direct expression of NOVX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: Manipulating the Mouse Embryo, Cold Spring Harbor Laboratory Press, Cold Spring Harbor, N.Y. Similar methods are used for production of other transgenic animals. A transgenic founder animal can be identified based upon the presence of the NOVX transgene in its genome and/or expression of NOVX mRNA in tissues or cells of the animals. A transgenic founder animal can then be used to breed additional animals carrying the transgene. Moreover, transgenic animals carrying a transgene-encoding NOVX protein can further be bred to other transgenic animals carrying other transgenes.

To create a homologous recombinant animal, a vector is prepared which contains at least a portion of a NOVX gene into which a deletion, addition or substitution has been introduced to thereby alter, e.g., functionally disrupt, the NOVX gene. The NOVX gene can be a human gene (e.g., the cDNA of any one of SEQ ID NO:2n−1, wherein n is an integer between 1-73), but more preferably, is a non-human homologue of a human NOVX gene. For example, a mouse homologue of human NOVX gene of SEQ ID NO:2n−1, wherein n is an integer between 1-73, can be used to construct a homologous recombination vector suitable for altering an endogenous NOVX gene in the mouse genome. In one embodiment, the vector is designed such that, upon homologous recombination, the endogenous NOVX gene is functionally disrupted (i.e., no longer encodes a functional protein; also referred to as a “knock out” vector).

Alternatively, the vector can be designed such that, upon homologous recombination, the endogenous NOVX gene is mutated or otherwise altered but still encodes functional protein (e.g., the upstream regulatory region can be altered to thereby alter the expression of the endogenous NOVX protein). In the homologous recombination vector, the altered portion of the NOVX gene is flanked at its 5′- and 3′-termini by additional nucleic acid of the NOVX gene to allow for homologous recombination to occur between the exogenous NOVX gene carried by the vector and an endogenous NOVX gene in an embryonic stem cell. The additional flanking NOVX nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced NOVX gene has homologously-recombined with the endogenous NOVX gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: Teratocarcinomas and Embryonic Stem Cells: A Practical Approach, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The NOVX nucleic acid molecules, NOVX proteins, and anti-NOVX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a NOVX protein or anti-NOVX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The isolated nucleic acid molecules of the invention can be used to express NOVX protein (e.g., via a recombinant expression vector in a host cell in gene therapy applications), to detect NOVX mRNA (e.g., in a biological sample) or a genetic lesion in a NOVX gene, and to modulate NOVX activity, as described further, below. In addition, the NOVX proteins can be used to screen drugs or compounds that modulate the NOVX protein activity or expression as well as to treat disorders characterized by insufficient or excessive production of NOVX protein or production of NOVX protein forms that have decreased or aberrant activity compared to NOVX wild-type protein (e.g.; diabetes (regulates insulin release); obesity (binds and transport lipids); metabolic disturbances associated with obesity, the metabolic syndrome X as well as anorexia and wasting disorders associated with chronic diseases and various cancers, and infectious disease(possesses anti-microbial activity) and the various dyslipidemias. In addition, the anti-NOVX antibodies of the invention can be used to detect and isolate NOVX proteins and modulate NOVX activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to NOVX proteins or have a stimulatory or inhibitory effect on, e.g., NOVX protein expression or NOVX protein activity. The invention also includes compounds identified in the screening assays described herein.

In one embodiment, the invention provides assays for screening candidate or test compounds which bind to or modulate the activity of the membrane-bound form of a NOVX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37: 1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of NOVX protein, or a biologically-active portion thereof, on the cell surface is contacted with a test compound and the ability of the test compound to bind to a NOVX protein determined. The cell, for example, can of mammalian origin or a yeast cell. Determining the ability of the test compound to bind to the NOVX protein can be accomplished, for example, by coupling the test compound with a radioisotope or enzymatic label such that binding of the test compound to the NOVX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of NOVX protein, or a biologically-active portion thereof, on the cell surface with a known compound which binds NOVX to form an assay mixture, contacting the assay mixture with a test compound, and determining the ability of the test compound to interact with a NOVX protein, wherein determining the ability of the test compound to interact with a NOVX protein comprises determining the ability of the test compound to preferentially bind to NOVX protein or a biologically-active portion thereof as compared to the known compound.

In another embodiment, an assay is a cell-based assay comprising contacting a cell expressing a membrane-bound form of NOVX protein, or a biologically-active portion thereof, on the cell surface with a test compound and determining the ability of the test compound to modulate (e.g., stimulate or inhibit) the activity of the NOVX protein or biologically-active portion thereof. Determining the ability of the test compound to modulate the activity of NOVX or a biologically-active portion thereof can be accomplished, for example, by determining the ability of the NOVX protein to bind to or interact with a NOVX target molecule. As used herein, a “target molecule” is a molecule with which a NOVX protein binds or interacts in nature, for example, a molecule on the surface of a cell which expresses a NOVX interacting protein, a molecule on the surface of a second cell, a molecule in the extracellular milieu, a molecule associated with the internal surface of a cell membrane or a cytoplasmic molecule. A NOVX target molecule can be a non-NOVX molecule or a NOVX protein or polypeptide of the invention. In one embodiment, a NOVX target molecule is a component of a signal transduction pathway that facilitates transduction of an extracellular signal (e.g. a signal generated by binding of a compound to a membrane-bound NOVX molecule) through the cell membrane and into the cell. The target, for example, can be a second intercellular protein that has catalytic activity or a protein that facilitates the association of downstream signaling molecules with NOVX.

Determining the ability of the NOVX protein to bind to or interact with a NOVX target molecule can be accomplished by one of the methods described above for determining direct binding. In one embodiment, determining the ability of the NOVX protein to bind to or interact with a NOVX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising a NOVX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting a NOVX protein or biologically-active portion thereof with a test compound and determining the ability of the test compound to bind to the NOVX protein or biologically-active portion thereof. Binding of the test compound to the NOVX protein can be determined either directly or indirectly as described above. In one such embodiment, the assay comprises contacting the NOVX protein or biologically-active portion thereof with a known compound which binds NOVX to form an assay mixture, contacting the assay mixture with a test compound, and determining the ability of the test compound to interact with a NOVX protein, wherein determining the ability of the test compound to interact with a NOVX protein comprises determining the ability of the test compound to preferentially bind to NOVX or biologically-active portion thereof as compared to the known compound.

In still another embodiment, an assay is a cell-free assay comprising contacting NOVX protein or biologically-active portion thereof with a test compound and determining the ability of the test compound to modulate (e.g. stimulate or inhibit) the activity of the NOVX protein or biologically-active portion thereof. Determining the ability of the test compound to modulate the activity of NOVX can be accomplished, for example, by determining the ability of the NOVX protein to bind to a NOVX target molecule by one of the methods described above for determining direct binding. In an alternative embodiment, determining the ability of the test compound to modulate the activity of NOVX protein can be accomplished by determining the ability of the NOVX protein further modulate a NOVX target molecule. For example, the catalytic/enzymatic activity of the target molecule on an appropriate substrate can be determined as described, supra.

In yet another embodiment, the cell-free assay comprises contacting the NOVX protein or biologically-active portion thereof with a known compound which binds NOVX protein to form an assay mixture, contacting the assay mixture with a test compound, and determining the ability of the test compound to interact with a NOVX protein, wherein determining the ability of the test compound to interact with a NOVX protein comprises determining the ability of the NOVX protein to preferentially bind to or modulate the activity of a NOVX target molecule.

The cell-free assays of the invention are amenable to use of both the soluble form or the membrane-bound form of NOVX protein. In the case of cell-free assays comprising the membrane-bound form of NOVX protein, it may be desirable to utilize a solubilizing agent such that the membrane-bound form of NOVX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl-N,N-dimethyl-3-ammonio-1-propane sulfonate, 3-(3-cholamidopropyl)dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either NOVX protein or its target molecule to facilitate separation of complexed from uncomplexed forms of one or both of the proteins, as well as to accommodate automation of the assay. Binding of a test compound to NOVX protein, or interaction of NOVX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-NOVX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or NOVX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of NOVX protein binding or activity determined using standard techniques.

Other techniques for immobilizing proteins on matrices can also be used in the screening assays of the invention. For example, either the NOVX protein or its target molecule can be immobilized utilizing conjugation of biotin and streptavidin. Biotinylated NOVX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with NOVX protein or target molecules, but which do not interfere with binding of the NOVX protein to its target molecule, can be derivatized to the wells of the plate, and unbound target or NOVX protein trapped in the wells by antibody conjugation. Methods for detecting such complexes, in addition to those described above for the GST-immobilized complexes, include immunodetection of complexes using antibodies reactive with the NOVX protein or target molecule, as well as enzyme-linked assays that rely on detecting an enzymatic activity associated with the NOVX protein or target molecule.

In another embodiment, modulators of NOVX protein expression are identified in a method wherein a cell is contacted with a candidate compound and the expression of NOVX mRNA or protein in the cell is determined. The level of expression of NOVX mRNA or protein in the presence of the candidate compound is compared to the level of expression of NOVX mRNA or protein in the absence of the candidate compound. The candidate compound can then be identified as a modulator of NOVX mRNA or protein expression based upon this comparison. For example, when expression of NOVX mRNA or protein is greater (i.e., statistically significantly greater) in the presence of the candidate compound than in its absence, the candidate compound is identified as a stimulator of NOVX mRNA or protein expression. Alternatively, when expression of NOVX mRNA or protein is less (statistically significantly less) in the presence of the candidate compound than in its absence, the candidate compound is identified as an inhibitor of NOVX mRNA or protein expression. The level of NOVX mRNA or protein expression in the cells can be determined by methods described herein for detecting NOVX mRNA or protein.

In yet another aspect of the invention, the NOVX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with NOVX (“NOVX-binding proteins” or “NOVX-bp”) and modulate NOVX activity. Such NOVX-binding proteins are also involved in the propagation of signals by the NOVX proteins as, for example, upstream or downstream elements of the NOVX pathway.

The two-hybrid system is based on the modular nature of most transcription factors, which consist of separable DNA-binding and activation domains. Briefly, the assay utilizes two different DNA constructs. In one construct, the gene that codes for NOVX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a NOVX-dependent complex, the DNA-binding and activation domains of the transcription factor are brought into close proximity. This proximity allows transcription of a reporter gene (e.g, LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with NOVX.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome. This process is called chromosome mapping. Accordingly, portions or fragments of the NOVX sequences of SEQ ID NO:2n−1, wherein n is an integer between 1-73, or fragments or derivatives thereof, can be used to map the location of the NOVX genes, respectively, on a chromosome. The mapping of the NOVX sequences to chromosomes is an important first step in correlating these sequences with genes associated with disease.

Briefly, NOVX genes can be mapped to chromosomes by preparing PCR primers (preferably 15-25 bp in length) from the NOVX sequences. Computer analysis of the NOVX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the NOVX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the NOVX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., Human Chromosomes: A Manual of Basic Techniques (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, Mendelian Inheritance in Man, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the NOVX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The NOVX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the NOVX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The NOVX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If coding sequences, such as those of SEQ ID NO:2n−1, wherein n is an integer between 1-73, are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining NOVX protein and/or nucleic acid expression as well as NOVX activity, in the context of a biological sample (e.g., blood, serum, cells, tissue) to thereby determine whether an individual is afflicted with a disease or disorder, or is at risk of developing a disorder, associated with aberrant NOVX expression or activity. The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers. The invention also provides for prognostic (or predictive) assays for determining whether an individual is at risk of developing a disorder associated with NOVX protein, nucleic acid expression or activity. For example, mutations in a NOVX gene can be assayed in a biological sample. Such assays can be used for prognostic or predictive purpose to thereby prophylactically treat an individual prior to the onset of a disorder characterized by or associated with NOVX protein, nucleic acid expression, or biological activity.

Another aspect of the invention provides methods for determining NOVX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of NOVX in clinical trials.

These and other agents are described in further detail in the following sections.

Diagnostic Assays

An exemplary method for detecting the presence or absence of NOVX in a biological sample involves obtaining a biological sample from a test subject and contacting the biological sample with a compound or an agent capable of detecting NOVX protein or nucleic acid (e.g., mRNA, genomic DNA) that encodes NOVX protein such that the presence of NOVX is detected in the biological sample. An agent for detecting NOVX mRNA or genomic DNA is a labeled nucleic acid probe capable of hybridizing to NOVX mRNA or genomic DNA. The nucleic acid probe can be, for example, a full-length NOVX nucleic acid, such as the nucleic acid of SEQ ID NO:2n−1, wherein n is an integer between 1-73, or a portion thereof, such as an oligonucleotide of at least 15, 30, 50, 100, 250 or 500 nucleotides in length and sufficient to specifically hybridize under stringent conditions to NOVX mRNA or genomic DNA. Other suitable probes for use in the diagnostic assays of the invention are described herein.

An agent for detecting NOVX protein is an antibody capable of binding to NOVX protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect NOVX mRNA, protein, or genomic DNA in a biological sample in vitro as well as in vivo. For example, in vitro techniques for detection of NOVX mRNA include Northern hybridizations and in situ hybridizations. In vitro techniques for detection of NOVX protein include enzyme linked immunosorbent assays (ELISAs), Western blots, immunoprecipitations, and immunofluorescence. In vitro techniques for detection of NOVX genomic DNA include Southern hybridizations. Furthermore, in vivo techniques for detection of NOVX protein include introducing into a subject a labeled anti-NOVX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting NOVX protein, mRNA, or genomic DNA, such that the presence of NOVX protein, mRNA or genomic DNA is detected in the biological sample, and comparing the presence of NOVX protein, mRNA or genomic DNA in the control sample with the presence of NOVX protein, mRNA or genomic DNA in the test sample.

The invention also encompasses kits for detecting the presence of NOVX in a biological sample. For example, the kit can comprise: a labeled compound or agent capable of detecting NOVX protein or mRNA in a biological sample; means for determining the amount of NOVX in the sample; and means for comparing the amount of NOVX in the sample with a standard. The compound or agent can be packaged in a suitable container. The kit can further comprise instructions for using the kit to detect NOVX protein or nucleic acid.

Prognostic Assays

The diagnostic methods described herein can furthermore be utilized to identify subjects having or at risk of developing a disease or disorder associated with aberrant NOVX expression or activity. For example, the assays described herein, such as the preceding diagnostic assays or the following assays, can be utilized to identify a subject having or at risk of developing a disorder associated with NOVX protein, nucleic acid expression or activity. Alternatively, the prognostic assays can be utilized to identify a subject having or at risk for developing a disease or disorder. Thus, the invention provides a method for identifying a disease or disorder associated with aberrant NOVX expression or activity in which a test sample is obtained from a subject and NOVX protein or nucleic acid (e.g, mRNA, genomic DNA) is detected, wherein the presence of NOVX protein or nucleic acid is diagnostic for a subject having or at risk of developing a disease or disorder associated with aberrant NOVX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant NOVX expression or activity. For example, such methods can be used to determine whether a subject can be effectively treated with an agent for a disorder. Thus, the invention provides methods for determining whether a subject can be effectively treated with an agent for a disorder associated with aberrant NOVX expression or activity in which a test sample is obtained and NOVX protein or nucleic acid is detected (e.g., wherein the presence of NOVX protein or nucleic acid is diagnostic for a subject that can be administered the agent to treat a disorder associated with aberrant NOVX expression or activity).

The methods of the invention can also be used to detect genetic lesions in a NOVX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding a NOVX-protein, or the misexpression of the NOVX gene. For example, such genetic lesions can be detected by ascertaining the existence of at least one of: (i) a deletion of one or more nucleotides from a NOVX gene; (ii) an addition of one or more nucleotides to a NOVX gene; (iii) a substitution of one or more nucleotides of a NOVX gene, (iv) a chromosomal rearrangement of a NOVX gene; (v) an alteration in the level of a messenger RNA transcript of a NOVX gene, (vi) aberrant modification of a NOVX gene, such as of the methylation pattern of the genomic DNA, (vii) the presence of a non-wild-type splicing pattern of a messenger RNA transcript of a NOVX gene, (viii) a non-wild-type level of a NOVX protein, (ix) allelic loss of a NOVX gene, and (x) inappropriate post-translational modification of a NOVX protein. As described herein, there are a large number of assay techniques known in the art which can be used for detecting lesions in a NOVX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the NOVX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to a NOVX gene under conditions such that hybridization and amplification of the NOVX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a NOVX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in NOVX can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med 2: 753-759. For example, genetic mutations in NOVX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the NOVX gene and detect mutations by comparing the sequence of the sample NOVX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the NOVX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type NOVX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S₁ nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in NOVX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on a NOVX sequence, e.g., a wild-type NOVX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in NOVX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control NOVX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a NOVX gene.

Furthermore, any cell type or tissue, preferably peripheral blood leukocytes, in which NOVX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on NOVX activity (e.g., NOVX gene expression), as identified by a screening assay described herein can be administered to individuals to treat (prophylactically or therapeutically) disorders (The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers.) In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of NOVX protein, expression of NOVX nucleic acid, or mutation content of NOVX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome pregnancy zone protein precursor enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of NOVX protein, expression of NOVX nucleic acid, or mutation content of NOVX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a NOVX modulator, such as a modulator identified by one of the exemplary screening assays described herein.

Monitoring of Effects During Clinical Trials

Monitoring the influence of agents (e.g., drugs, compounds) on the expression or activity of NOVX (e.g., the ability to modulate aberrant cell proliferation and/or differentiation) can be applied not only in basic drug screening, but also in clinical trials. For example, the effectiveness of an agent determined by a screening assay as described herein to increase NOVX gene expression, protein levels, or upregulate NOVX activity, can be monitored in clinical trails of subjects exhibiting decreased NOVX gene expression, protein levels, or downregulated NOVX activity. Alternatively, the effectiveness of an agent determined by a screening assay to decrease NOVX gene expression, protein levels, or downregulate NOVX activity, can be monitored in clinical trails of subjects exhibiting increased NOVX gene expression, protein levels, or upregulated NOVX activity. In such clinical trials, the expression or activity of NOVX and, preferably, other genes that have been implicated in, for example, a cellular proliferation or immune disorder can be used as a “read out” or markers of the immune responsiveness of a particular cell.

By way of example, and not of limitation, genes, including NOVX, that are modulated in cells by treatment with an agent (e.g., compound, drug or small molecule) that modulates NOVX activity (e.g., identified in a screening assay as described herein) can be identified. Thus, to study the effect of agents on cellular proliferation disorders, for example, in a clinical trial, cells can be isolated and RNA prepared and analyzed for the levels of expression of NOVX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of NOVX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of a NOVX protein, mRNA, or genomic DNA in the preadministration sample; (iii) obtaining one or more post-administration samples from the subject; (iv) detecting the level of expression or activity of the NOVX protein, mRNA, or genomic DNA in the post-administration samples; (v) comparing the level of expression or activity of the NOVX protein, mRNA, or genomic DNA in the pre-administration sample with the NOVX protein, mRNA, or genomic DNA in the post administration sample or samples; and (vi) altering the administration of the agent to the subject accordingly. For example, increased administration of the agent may be desirable to increase the expression or activity of NOVX to higher levels than detected, i.e., to increase the effectiveness of the agent. Alternatively, decreased administration of the agent may be desirable to decrease expression or activity of NOVX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant NOVX expression or activity. The disorders include cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, adrenoleukodystrophy, congenital adrenal hyperplasia, prostate cancer, neoplasm; adenocarcinoma, lymphoma, uterus cancer, fertility, hemophilia, hypercoagulation, idiopathic thrombocytopenic purpura, immunodeficiencies, graft versus host disease, AIDS, bronchial asthma, Crohn's disease; multiple sclerosis, treatment of Albright Hereditary Ostoeodystrophy, and other diseases, disorders and conditions of the like.

These methods of treatment will be discussed more fully, below.

Disease and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (see, e.g., Capecchi, 1989. Science 244: 1288-1292); or (v) modulators (i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof, or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant NOVX expression or activity, by administering to the subject an agent that modulates NOVX expression or at least one NOVX activity. Subjects at risk for a disease that is caused or contributed to by aberrant NOVX expression or activity can be identified by, for example, any or a combination of diagnostic or prognostic assays as described herein. Administration of a prophylactic agent can occur prior to the manifestation of symptoms characteristic of the NOVX aberrancy, such that a disease or disorder is prevented or, alternatively, delayed in its progression. Depending upon the type of NOVX aberrancy, for example, a NOVX agonist or NOVX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating NOVX expression or activity for therapeutic purposes. The modulatory method of the invention involves contacting a cell with an agent that modulates one or more of the activities of NOVX protein activity associated with the cell. An agent that modulates NOVX protein activity can be an agent as described herein, such as a nucleic acid or a protein, a naturally-occurring cognate ligand of a NOVX protein, a peptide, a NOVX peptidomimetic, or other small molecule. In one embodiment, the agent stimulates one or more NOVX protein activity. Examples of such stimulatory agents include active NOVX protein and a nucleic acid molecule encoding NOVX that has been introduced into the cell. In another embodiment, the agent inhibits one or more NOVX protein activity. Examples of such inhibitory agents include antisense NOVX nucleic acid molecules and anti-NOVX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of a NOVX protein or nucleic acid molecule. In one embodiment, the method involves administering an agent (e.g., an agent identified by a screening assay described herein), or combination of agents that modulates (e.g., up-regulates or down-regulates) NOVX expression or activity. In another embodiment, the method involves administering a NOVX protein or nucleic acid molecule as therapy to compensate for reduced or aberrant NOVX expression or activity.

Stimulation of NOVX activity is desirable in situations in which NOVX is abnormally downregulated and/or in which increased NOVX activity has a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

The NOVX nucleic acids and proteins of the invention are useful in potential prophylactic and therapeutic applications implicated in a variety of disorders including, but not limited to: metabolic disorders, diabetes, obesity, infectious disease, anorexia, cancer-associated cancer, neurodegenerative disorders, Alzheimer's Disease, Parkinson's Disorder, immune disorders, hematopoietic disorders, and the various dyslipidemias, metabolic disturbances associated with obesity, the metabolic syndrome X and wasting disorders associated with chronic diseases and various cancers.

As an example, a cDNA encoding the NOVX protein of the invention may be useful in gene therapy, and the protein may be useful when administered to a subject in need thereof. By way of non-limiting example, the compositions of the invention will have efficacy for treatment of patients suffering from: metabolic disorders, diabetes, obesity, infectious disease, anorexia, cancer-associated cachexia, cancer, neurodegenerative disorders, Alzheimer's Disease, Parkinson's Disorder, immune disorders, hematopoietic disorders, and the various dyslipidemias.

Both the novel nucleic acid encoding the NOVX protein, and the NOVX protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies, which immunospecifically-bind to the novel substances of the invention for use in therapeutic or diagnostic methods.

### EXAMPLES

### Example A: Polynucleotide and Polypeptide Sequences, and Homology Data

### Example 1

The NOV1 clone was analyzed, and the nucleotide and encoded polypeptide sequences are shown in Table 1A.

TABLE 1A
NOV1 Sequence Analysis

| | |
|---|---|
| SEQ ID NO:1 | 3430 bp |

| | |
|---|---|
| NOV1a, | GGGCTGCAGGAATTCCCCCACAGAGGGAGCATGACTTCGGCAACTTCACCTATCATTC |
| CG100653-01 | |
| DNA Sequence | TGAAATGGGACCCCAAAAGTTTGGAAATCCGGACGCTAACAGTGGAAAGGCTGTTGGA |
| | GCCACTTGTTACACAGGTGACTACACTTGTCAACACAAGCAACAAAGGCCCATCTGGT |
| | AAAAAGAAAGGGAGGTCAAAGAAAGCCCATGTACTAGCTGCCTCTGTAGAGCAAGCCA |
| | CTCAGAATTTCCTGGAAAAGGGTGAACAGATCGCTAAGGAGAGTCAAGATCTCAAAGA |
| | AGAGTTGGTGGCTGCTGTAGAGGATGTGCGCAAACAAGGTGAGACGATGCGGATCGCC |
| | TCCTCCGAGTTTGCAGATGACCCTTGCTCGTCGGTAAAGCGCGGCACCATGGTACGGG |
| | CGGCAAGGGCTTTGCTCTCCGCGGTGACACGCTTACTCATCCTGGCGGACATGGCAGA |
| | TGTCATGAGACTTTTATCCCATCTGAAAATTGTGGAAGAGGCCCTGGAAGCTGTCAAA |
| | AATGCTACAAATGAGCAAGACCTTGCAAACCGTTTTAAAGAGTTTGGGAAAAAGATGG |
| | TGAAACTTAACTATGTAGCAGCAAGAAGACAACAGGAGCTGAAGGATCCTCACTGTCG |
| | GGATGAGATGGCAGCCGCCCGAGGGGCTCTGAAGAAGAATGCCACAATGCTGTACACG |
| | GCCTCTCAAGCATTTCTCCGCCACCCAGATGTCGCCGCTACGAGAGCCAACCGAGATT |
| | ATGTGTTCAAACAAGTCCAGGAGGCCATCGCCGGCATCTCCAATGCTGCTCAAGCTAC |
| | CTCGCCCACTGACGAAGCCAAGGGCCACACGGGCATCGGCGAGCTGGCTGCGGCTCTT |
| | AATGAGTTTGACAATAAGATTATCCTGGACCCCATGACGTTCAGCGAGGCCAGGTTCC |
| | GGCCGTCCCTGGAGGAGAGGCTGGAGAGCATCATCAGCGGCGCAGCGCTGATGGCCGA |
| | CTCCTCCTGCACGCGAGACGACCGGCGCGAGAGGATCGTGGCGGAGTGCAACGCCGTG |
| | CGGCAGGCGCTCCAGGACCTGCTCAGCGAGTACATGAATAATACTGGAAGGAAAGAAA |
| | GAGACAGCTTCGGAAAGCAGTGATGGATCACATATCTGACTCTTTCCTGGAAACCAAT |
| | GTTCCTTTGCTAGTTCTCATTGAGGCTGCAAAGAGCGGAAATGAAAAGGAAGTGAAAG |
| | AATATGCCCAAGTTTTCCGTGAGCATGCCAACAAACTGGTAGAGGTTGCCAATTTGGC |
| | CTGTTCCATCTCCAACAATGAASAAGGGGTGAAATTAGTTCGGATGGCAGCCACCCAG |
| | ATTGACAGCCTGTGTCCCCAGGTCATCAATGCCGCTCTGACACTGGCTGCCCGGCCAC |
| | AGAGCAAAGTTGCTCAGGATAACATGGACGTCTTCAAAGACCAGTGGGAGAAGCAGGT |
| | CCGAGTGTTGACAGAGGCCGTGGATGACATCACCTCAGTGGATGACTTCCTCTCTGTC |
| | TCAGAAAATCACATCTTGGAGGATGTGAACAAGTGTGTGATAGCCCTCCAAGAGGGCG |
| | ATGTGGACACTCTGGACCGGACTGCAGGGGCCATCAGGGGCCGGGCAGCTCGAGTCAT |
| | ACACATCATCAATGCTGAGATGGAGAACTATGAAGCTGGGGTTTATACTGAGAAGGTG |
| | TTGGAAGCTACAAAATTGCTTTCTGAAACAGTGATGCCACGCTTCGCTGAACAAGTAG |
| | AGGTTGCCATTGAAGCCCTGAGTGCCAACGTTCCTCAACCGTTTGAGGAGAATGAGTT |
| | CATCGATGCCTCTCGCCTGGTGTATGATGGCGTTCGGGACATCAGAAAGGCTGTGCTG |
| | ATGATCAGGACCCCAGAAGAACTAGAGGATGATTCTGACTTTGAGCAGGAAGATTATG |
| | ATGTGCGTAGAGGGACAAGTGTTCAGACTGAGGATGACCAGCTCATTGCAGGGCAGAG |
| | CGCACGGGCCATCATGGCGCAACTACCGCAGGAGGAGAAGGCAAAAATAGCTGAGCAG |
| | GTGGAGATATTCCATCAAGAGAAAAGCAAGCTGGATGCAGAAGTGGCCAAATGGGACG |
| | ACAGCGGCAATGATATCATTGTACTGGCCAGCAGATGTGTATGATCATGATGGGAAAT |
| | GACAGACTTCACAAGAGGCAAAGGCCCATTGAAAAATACATCTGATGTCATTAATGCT |
| | GCCAAGAAAATTGCCGAAGCAGGTTCTCGAATGGACAAATTAGCTCGTGCTGTGGCTG |
| | ATCAGCTGGACAGTGCCACATCGCTTATCCAGGCAGCTAAAAACCTGATGAATGCTGT |
| | TGTCCTCACGGTGAAAGCATCCTATGTGGCCTCAACCAAATACCAGAAGGTCTATGGG |
| | ACAGCAGCTGTCAACTCACCTGTTGTGTCTTGGAAGATGAAGGCTCCAGAGAAGAAGC |
| | CCCTTGTGAAGAGAGAAAAGCCTGAAGAATTCCAGACACGAGTTCGACGAGGTTCTCA |
| | GAAGAAACACATTTCGCCTGTACAGGCTTTAAQTGAATTCAAAGCAATGGATTCCTTC |
| | TAGGACGATAGGTTTTAACAAGAAAGCTTTTTCTTTCTTTTCTTTCTTTCTTTTTCTT |
| | TTTAATTCCATTTTTGTATGCATACCTGCCAGCTCGTATGCCTCTGGCATGGGGAAAT |
| | TAAGGGAACAGTGTCTGTTTGCATGTAAGATGAGATGAGATCAATACTACTGATCCAT |
| | CTGTACCCTGCGAAGGAGACAGGACATTCCTGTACTAAGGTGGCACAGAGCTGTCCTT |
| | TGCAACATTCTCATAATATTGGGCACAGAGTTCGCATTGGCGCAATATTTATGGGAGT |
| | GGGAGGGATGGGGAAAATAAACTTAACTCTACAAAAGCAAACTCTAATGCATGCAAGA |
| | ATCATTAGGTTGGCAGGTATATGCATAAGTGAAAAATCTGGAAGTGTAATGGTAGAAC |
| | ATAAAACTTGTATTGCTTCTGTTTCAGTGCAAAAATGTACTAGCCAATACGCTTAAGT |
| | GTGTGGCCCATGAATTGAACAATTTAACCTTCAAGTCTATATCCGTGATATTATGTCG |
| | ATTTTTAACTGAGGGGAAATTAACTAGTCCAGCCTAAAATGCTTCTTTTAATCTGCAT |
| | TCTGTTTCCTCTTCTAGTTGTGCCATTACTAGTGATCATGTTTTTTTCCCCCCTTTAA |
| | TGAAAACAATAAACATCTATTTGAGACAATTAAAATCCTTCTGGGGGCACTGGAAGCA |
| | CAATACGGTGACCAATCTTGCTTTCATTTTTTTTTCTTTTTAATTTGAACCATGATTT |
| | TGCTAGAAATAGAAGGCCCAGTGGTGGAATATTAGAGGGAAGGAAACTGACAACGTGT |
| | GAAAGTTA |

| | | |
|---|---|---|
| ORE Start: ATG at 31 | | ORF Stop: TAG at 2611 |
| SEQ ID NO: 2 | 860 aa | MW at 95525.9 kD |

| | |
|---|---|
| NOV1a, | MTSATSPIILKWDPKSLEIRTLTVERLLEPLVTQVTTLVNTSNKGPSGKKKGRSKKAH |
| CG100653-01 | |
| Protein Sequence | VLAASVEQATONFLEKGEQIAKESQDLKEELVAAVEDVRKQGETMRIASSEFADDPCS |
| | SVKRGTMVRAARALLSAVTRLLILADMADVMRLLSHLKIVEEALEAVKNATNEQDLAN |
| | RFKEFGKKMVKLNYVAARRQQELKDPHCRDEMAAARGALKKNATMLYTASQAFLRHPD |
| | VAATRANRDYVFKQVQEAIAGISNAAQATSPTDEAKGHTGIGELAAALNEFDNKIILD |
| | PMTFSEARFRPSLEERLESIISGAALMADSSCTRDDRRERIVAECNAVRQALQDLLSE |
| | YMNNTGRKEKGDPLNIAIDKMTKKTRDLRRQLRKAVMDHISDSFLETNVPLLVLIEAA |
| | KSGNEKEVKEYAQVFREHANKLVEVANLACSISNNEEGVKLVRMAATQIDSLCPQVIN |
| | AALTLAARPQSKVAQDNMDVFKDQWEKQVRVLTEAVDDITSVDDFLSVSENHILEDVN |
| | KCVIALQEGDVDTLDRTAGAIRGRAARVIHIINAEMENYEAGVYTEKVLEATKLLSET |
| | VMPRFAEQVEVAIEALSANVPQPFEENEFIDASRLVYDGVRDIRKAVLMIRTPEELED |
| | DSDFEQEDYDVRRGTSVQTEDDQLIAGQSARAIMAQLPQEEKAKIAEQVEIFHQEKSK |
| | LDAEVAKWDDSGNDIIVLAKQMCMIMMEMTDFTRGKGPLKNTSDVINAAKKIAEAGSR |
| | MDKLARAVADQLDSATSLIQAAKNLMNAVVLTVKASYVASTKYQKVYGTAAVNSPVVS |
| | WKMKAPEKKPLVKREKPEEFQTRVRRGSQKKHISPVQALSEFKAMDSF |

Further analysis of the NOV1a protein yielded the following properties shown in Table 1B.

TABLE 1B
Protein Sequence Properties NOV1a

| | |
|---|---|
| PSort | 0.3600 probability located in mitochondrial matrix space; |
| analysis: | 0.3000 probability located in microbody (peroxisome); 0.1000 |
| | probability located in lysosome (lumen); 0.0000 probability |
| | located in endoplasmic reticulum (membrane) |
| SignalP | No Known Signal Sequence Predicted |
| analysis: | |

A search of the NOV1a protein against the Geneseq database, a proprietary database that contains sequences published in patents and patent publication, yielded several homologous proteins shown in Table 1C.

TABLE 1C
Geneseq Results for NOV1a

| | | NOV1a | Identities/ | |
|---|---|---|---|---|
| | | Residues/ | Similarities for | |
| Geneseq | Protein/Organism/Length [Patent | Match | the Matched | Expect |
| Identifier | #, Date] | Residues | Region | Value |
| AAR58778 | Neural alpha-catenin protein - | 1 . . . 860 | 851/906 (93%) | 0.0 |
| | Homo sapiens, 906 aa. | 1 . . . 906 | 855/906 (93%) | |
| | [JP06211898-A, 02-AUG-1994] | | | |
| AAY07060 | Renal cancer associated antigen | 8 . . . 859 | 694/899 (77%) | 0.0 |
| | precursor sequence - Homo sapiens, | 9 . . . 905 | 773/899 (85%) | |
| | 906 aa. [WO9904265-A2, 28-JAN-1999] | | | |
| AAU32945 | Novel human secreted protein | 8 . . . 769 | 611/766 (79%) | 0.0 |
| | #3436 - Homo sapiens, 932 aa. | 10 . . . 773 | 683/766 (88%) | |
| | [WO200179449-A2, 25-OCT-2001] | | | |
| ABG10622 | Novel human diagnostic protein | 8 . . . 769 | 610/766 (79%) | 0.0 |
| | #10613 - Homo sapiens, 932 aa. | 10 . . . 773 | 682/766 (88%) | |
| | [WO200175067-A2, 11-OCT-2001] | | | |
| ABG10622 | Novel human diagnostic protein | 8 . . . 769 | 610/766 (79%) | 0.0 |
| | #10613 - Homo sapiens, 932 aa. | 10 . . . 773 | 682/766 (88%) | |
| | [WO200175067-A2, 11-OCT-2001] | | | |

In a BLAST search of public sequence databases, the NOV1a protein was found to have homology to the proteins shown in the BLASTP data in Table 1D.

TABLE 1D
Public BLASTP Results for NOV1a

| | | NOV1a | Identities/ | |
|---|---|---|---|---|
| Protein | | Residues/ | Similarities for the | |
| Accession | | Match | Matched | Expect |
| Number | Protein/Organism/Length | Residues | Portion | Value |
| P30997 | Alpha-2 catenin (Alpha N-catenin) | 1 . . . 860 | 851/906 (93%) | 0.0 |
| | (Neural alpha-catenin) - Gallus | 1 . . . 906 | 855/906 (93%) | |
| | gallus (Chicken), 906 aa. | | | |
| I49499 | alpha N-catenin I - mouse, 905 aa. | 1 . . . 860 | 850/905 (93%) | 0.0 |
| | | 1 . . . 905 | 854/905 (93%) | |
| A45011 | alpha-catenin 2 - human, 945 aa. | 1 . . . 769 | 768/770 (99%) | 0.0 |
| | | 1 . . . 770 | 768/770 (99%) | |
| P26232 | Alpha-2 catenin (Alpha-catenin | 1 . . . 769 | 768/770 (99%) | 0.0 |
| | related protein) (Alpha N-catenin) - | 1 . . . 770 | 768/770 (99%) | |
| | Homo sapiens (Human), 953 aa. | | | |
| Q61301 | Alpha-2 catenin (Alpha-catenin | 1 . . . 769 | 759/770 (98%) | 0.0 |
| | related protein) (Alpha N-catenin) - | 1 . . . 770 | 762/770 (98%) | |
| | Mus musculus (Mouse), 953 aa. | | | |

PFam analysis predicts that the NOV1a protein contains the domains shown in the Table 1E.

TABLE 1E
Domain Analysis of NOV1a

| | | Identities/ | |
|---|---|---|---|
| | NOV1a | Similarities for | |
| Pfam Domain | Match Region | the Matched Region | Expect Value |
| Vinculin | 18 . . . 765 | 424/948 (45%) | 0 |
| | | 736/948 (78%) | |
| Vinculin | 766 . . . 821 | 32/57 (56%) | 5.4e−30 |
| | | 56/57 (98%) | |

### Example 2.

The NOV2 clone was analyzed, and the nucleotide and encoded polypeptide sequences are shown in Table 2A.

TABLE 2A
NOV2 Sequence Analysis

| | |
|---|---|
| SEQ ID NO:3 | 2883 bp |

| | |
|---|---|
| NOV2a, | CGTGAATGGTGTAGTGAGTTCTAATGAAACTTTATTTACAAGAGGAGACTGACCAGGT |
| CG100689-01 | |
| DNA Sequence | TTGGCCTGGGGGCCACAGTGTGTAGACCCCTGGAAAGATACATCCTGAGAAGAAAAAA |
| | AGAATATATGCAGGAATGCTTAACTTTGTGGGTTTTCTCTCCTCTTGCCCTCACTGAC |
| | TCAGGATACACAAAGACCTATCAAGCTCACGCAAAGCAGAAATTCAGCCGCTTATGGT |
| | CCAGCAAGTCTGTCACTGAGATTCACCTATACTTTGAGGAGGAAGTCAAGCAAGAAGA |
| | ATGTGACCATTTGGACCGCCTTTTTGCTCCCAAGGAAGCTGGGAAACAGCCACGTACA |
| | GTGATCATTCAAGGACCACAAGGAATTGGAAAAACGACACTCCTGATGAAGCTGATGA |
| | TGGCCTGGTCGGACAACAAGATCTTTCGGGATAGGTTCCTGTACACGTTCTATTTCTG |
| | CTGCAGAGAACTGAGGGAGTTGCCGCCAACGAGTTTGGCTGACTTGATTTCCAGAGAG |
| | TGGCCTGACCCCGCTGCTCCTATAACAGAGATCGTGTCTCAACCGGAGAGACTCTTGT |
| | TCGTCATCGACAGCTTCGAAGAGCTGCAGGGCGGCTTGAACGAACCCGATTCGGATCT |
| | GTGTGGTGACTTGATGGAGAAACGGCCGGTGCAGGTGCTTCTGAGCAGTTTGCTGAGG |
| | AAGAAGATGCTCCCGGAGGCCTCCCTGCTCATCGCTATCAAACCCGTGTGCCCGAAGG |
| | AGCTCCGGGATCAGGTGACGATCTCAGAAATCTACCAGCCCCGGGGATTCAACGAGAG |
| | TGATAGGTTAGTGTATTTCTGCTGTTTCTTCAAAGACCCGAAAAGAGCCATGGAAGCC |
| | TTCAATCTTGTAAGAGAAAGTGAACAGCTGTTTTCCATATGCCAAATCCCGCTCCTCT |
| | GCTGGATCCTGTGTACCAGTCTGAAGCAAGAGATGCAGAAAGGAAAAGACCTGGCCCT |
| | GACCTGCCAGAGCACTACCTCTGTGTACTCCTCTTTCGTCTTTAACCTGTTCACACCT |
| | GAGGGTGCCGAGGGCCCGACTCCGCAAACCCAGCACCAGCTGAAGGCCCTGTGCTCCC |
| | TGGCTGCAGAGGGTATGTGGACAGACACATTTGAGTTTTGTGAAGACGACCTCCGGAG |
| | AAATGGGGTTGTTGACGCTGACATCCCTGCGCTGCTGGGCACCAAGATACTTCTGAAG |
| | TACGGGGAGCGTGAGAGCTCCTACGTGTTCCTCCACGTGTGTATCCAGGAGTTCTGTG |
| | CCGCCTTGTTCTATTTGCTCAAGAGCCACCTTGATCATCCTCACCCAGCTGTGAGATG |
| | TGTACAGGAATTGCTAGTTGCCAATTTTGAAAAAGCAAGGACAGCACATTGGATTTTT |
| | TTGGGGTGTTTTCTAACTGGCCTTTTAAATAAAAAGGAACAAGAAAAACTGGATGCGT |
| | TTTTTGGCTTCCAACTGTCCCAAGAGATAAAGCAGCAAATTCACCAGTGCCTGAAGAG |
| | CTTAGGGGAGCGTGGCAATCCTCAGGGACAGGTGGATTCCTTGGCGATATTTTACTGT |
| | CTCTTTGAAATGCAGGATCCTGCCTTTGTGAAGCAGGCAGTGAACCTCCTCCAAGAAG |
| | CTAACTTTCATATTATTGACAACGTGGACTTGGTGGTTTCTGCCTACTGCTTAAAATA |
| | CTGCTCCAGCTTGAGGAAACTCTGTTTTTCCGTTCAAAATGTCTTTAAGAAAGAGGAT |
| | GAACACAGCTCTACGTCGGATTACAGCCTCATCTGTTGGCATCACATCTGCTCTGTGC |
| | TCACCACCAGCGGGCACCTCAGAGAGCTCCAGGTGCAGGACAGCACCCTCAGCGAGTC |
| | GACCTTTGTGACCTGGTGTAACCAGCTGAGGCATCCCAGCTGTCGCCTTCAGAAGCTT |
| | GGAATAAATAACGTTTCCTTTTCTGGCCAGAGTGTTCTGCTCTTTGAGGTGCTCTTTT |
| | ATCAGCCAGACTTGAAATACCTGAGCTTCACCCTCACGAAACTCTCTCGTGATGACAT |
| | CAGGTCCCTCTGTGATGCCTTGAACTACCCAGCAGGCAACGTCAAAGAGCTAGCGCTG |
| | GTAAATTGTCACCTCTCACCCATTGATTGTGAAGTCCTTGCTGGCCTTCTAACCAACA |
| | ACAAGAAGCTGACGTATCTGAATGTATCCTGCAACCAGTTAGACACAGGCGTGCCCCT |
| | TTTGTGTGAASCCCTGTGCAGCCCAGACACGGTCCTGGTATACCTGATGTTGGCTTTC |
| | TGCCACCTCAGCGAGCAGTGCTGCGAATACATCTCTGAAATGCTTCTGCGTAACAAGA |
| | GCGTGCGCTATCTAGACCTCAGTGCCAATGTCCTGAAGGACGAAGGACTGAAAACTCT |
| | CTGCGAGGCCTTGAAACATCCGGACTGCTGCCTGGATTCACTGTGTTTGGTAAAATGT |
| | TTTATCACTGCTGCTGGCTGTGAAGACCTCGCCTCTGCTCTCATCAGCAATCAAAACC |
| | TGAAGATTCTGCAAATTGGGTGCAATGAAATCGGAGATGTGGGTGTGCAGCTGTTGTG |
| | TCGGGCTCTGACGCATACGGATTGCCGCTTAGAGATTCTTGGGTTGGAAGAATGTGGG |
| | TTAACGAGCACCTGCTGTAAGGATCTCGCGTCTGTTCTCACCTGCAGTAAGACCCTGC |
| | AGCAGCTCAACCTGACCTTGAACACCTTGGACCACACAGGGGTGGTTGTACTCTGTGA |
| | GGCCCTGAGACACCCAGAGTGTGCCCTGCAGGTGCTCGGGCTGAGAAAAACTGATTTT |
| | GATGAGGAAACCCAGGCACTTCTGACGGCTGAGGAAGAGAGAAATCCTAACCTGACCA |
| | TCACAGACGACTGTGACACAATCACAAGGGTAGAGATCTGA |

| | | |
|---|---|---|
| ORF Start: ATG at 124 | | ORF Stop: TGA at 2881 |
| SEQ ID NO:4 | 919 aa | MW at 103966.7 kD |

| | |
|---|---|
| NOV2a, | MQECLTLWVFSPLALTDSGYTKTYQAHAKQKFSRLWSSKSVTEIHLYFEEEVKQEECD |
| CG100689-01 | |
| Protein Sequence | HLDRLFAPKEAGKQPRTVIIQGPQGIGKTTLLMKLMMAWSDNKIFRDRFLYTFYFCCR |
| | ELRELPPTSLADLISREWPDPAAPITEIVSQPERLLFVIDSFEELQGGLNEPDSDLCG |
| | DLMEKRPVQVLLSSLLRKKMLPEASLLIAIKPVCPKELRDQVTISEIYQPRGFNESDR |
| | LVYFCCFFKDPKRAMEAFNLVRESEQLFSICQIPLLCWILCTSLKQEMQKGKDLALTC |
| | QSTTSVYSSFVFNLFTPEGAEGPTPQTQHQLKALCSLAAEGMWTDTFEFCEDDLRRNG |
| | VVDADIPALLGTKILLKYGERESSYVFLHVCIQEFCAALFYLLKSHLDHPHPAVRCVQ |
| | ELLVANFEKARRAHWIFLGCFLTGLLNKKEQEKLDAFFGFQLSQEIKQQIHQCLKSLG |
| | ERGNPQGQVDSLAIFYCLFEDQDPAFVKQAVNLLQEANFHIIDNVDLVVSAYCLKYCS |
| | SLRKLCFSVQNVFKKEDEHSSTSDYSLICWHHICSVLTTSGHLRELQVQDSTLSESTF |
| | VTWCNQLRHPSCRLQKLGINNVSFSGQSVLLFEVLFYQPDLKYLSFTLTKLSRDDIRS |
| | LCDALNYPAGNVKELALVNCHLSPIDCEVLAGLLTNNKKLTYLNVSCNQLDTGVPLLC |
| | EALCSPDTVLVYLMLAFCHLSEQCCEYISEMLLRNKSVRYLDLSANVLKDEGLKTLCE |
| | ALKHPDCCLDSLCLVKCFITAAGCEDLASALISNQNLKILQIGCNEIGDVGVQLLCRA |
| | LTHTDCRLEILGLEECGLTSTCCKDLASVLTCSKTLQQLNLTLNTLDHTGVVVLCEAL |
| | RHPECALQVLGLRKTDFDEETQALLTAEEERNPNLTITDDCDTITRVEI |

Further analysis of the NOV2a protein yielded the following properties shown in Table 2B.

TABLE 2B
Protein Sequence Properties NOV2a

| | |
|---|---|
| PSort | 0.6000 probability located in nucleus; 0.3000 probability |
| analysis: | located in microbody (peroxisome); 0.2000 probability located |
| | in endoplasmic reticulum (membrane); |
| | 0.1000 probability located in mitochondrial inner membrane |
| SignalP | Cleavage site between residues 17 and 18 |
| analysis: | |

A search of the NOV2a protein against the Geneseq database, a proprietary database that contains sequences published in patents and patent publication, yielded several homologous proteins shown in Table 2C.

TABLE 2C
Geneseq Results for NOV2a

| | | NOV2a | Identities/ | |
|---|---|---|---|---|
| | | Residues/ | Similarities for | |
| Geneseq | Protein/Organism/Length [Patent | Match | the Matched | Expect |
| Identifier | #, Date] | Residues | Region | Value |
| AAM50328 | Human nucleotide binding site | 33 . . . 882 | 849/850 (99%) | 0.0 |
| | protein NBS-5 - Homo sapiens, 858 | 1 . . . 850 | 850/850 (99%) | |
| | aa. [WO200183753-A2, 08-NOV-2001] | | | |
| AAU07878 | Polypeptide sequence for | 165 . . . 907 | 375/743 (50%) | 0.0 |
| | mammalian Spg65 - Mammalia, | 5 . . . 744 | 528/743 (70%) | |
| | 748 aa. [WO200166752-A2, 13-SEP-2001] | | | |
| AAE07514 | Human PYRIN-1 protein - Homo | 20 . . . 907 | 320/926 (34%) | e−146 |
| | sapiens, 1034 aa. [WO200161005- | 134 . . . 1028 | 491/926 (52%) | |
| | A2, 23-AUG-2001] | | | |
| AAG65895 | Amino acid sequence of GSK gene | 75 . . . 907 | 301/849 (35%) | e−137 |
| | Id 97078 - Homo sapiens, 1062 aa. | 208 . . . 1043 | 460/849 (53%) | |
| | [WO200172961-A2, 04-OCT-2001] | | | |
| AAE07513 | Human nucleotide binding site 1 | 75 . . . 907 | 299/849 (35%) | e−134 |
| | (NBS-1) protein - Homo sapiens, | 180 . . . 1014 | 459/849 (53%) | |
| | 1033 aa. [WO200161005-A2, 23-AUG-2001] | | | |

In a BLAST search of public sequence databases, the NOV2a protein was found to have homology to the proteins shown in the BLASTP data in Table 2D.

TABLE 2D
Public BLASTP Results for NOV2a

| | | | Identities/ | |
|---|---|---|---|---|
| | | NOV2a | Similarities | |
| Protein | | Residues/ | for the | |
| Accession | | Match | Matched | Expect |
| Number | Protein/Organism/Length | Residues | Portion | Value |
| Q96MN2 | CDNA FLJ32126 FIS, CLONE | 1 . . . 919 | 918/919 (99%) | 0.0 |
| | PEBLM2000112, WEAKLY | 1 . . . 919 | 918/919 (99%) | |
| | SIMILAR TO HOMO SAPIENS | | | |
| | NUCLEOTIDE-BINDING SITE | | | |
| | PROTEIN 1 MRNA - Homo sapiens | | | |
| | (Human), 919 aa. | | | |
| Q96MN2 | NACHT-, LRR- and PYD-containing | 18 . . . 919 | 900/902 (99%) | 0.0 |
| | protein 4 (PAAD and NACHT- | 93 . . . 994 | 901/902 (99%) | |
| | containing protein 2) (PYRIN- | | | |
| | containing APAF1-like protein 4) | | | |
| | (Ribonuclease inhibitor 2) - Homo | | | |
| | sapiens (Human), 994 aa. | | | |
| AAL88672 | RIBONUCLEASE INHIBITOR 2 - | 18 . . . 919 | 894/902 (99%) | 0.0 |
| | Homo sapiens (Human), 916 aa. | 15 . . . 916 | 897/902 (99%) | |
| CAD19386 | SEQUENCE 7 FROM PATENT | 33 . . . 882 | 849/850 (99%) | 0.0 |
| | WO0183753 - Homo sapiens (Human), | 1 . . . 850 | 850/850 (99%) | |
| | 858 aa (fragment). | | | |
| Q99MW0 | RIBONUCLEASE/ANGIOGENIN | 165 . . . 907 | 374/743 (50%) | 0.0 |
| | INHIBITOR 2 - Mus musculus | 5 . . . 744 | 528/743 (70%) | |
| | (Mouse), 748 aa. | | | |

PFam analysis predicts that the NOV2a protein contains the domains shown in the Table 2E.

TABLE 2E
Domain Analysis of NOV2a

| | | Identities/ | |
|---|---|---|---|
| | NOV2a | Similarities for | |
| Pfam Domain | Match Region | the Matched Region | Expect Value |
| SRP54 | 71 . . . 93 | 11/23 (48%) | 0.18 |
| | | 17/23 (74%) | |

### Example 3

The NOV3 clone was analyzed, and the nucleotide and encoded polypeptide sequences are shown in Table 3A.

TABLE 3A
NOV3 Sequence Analysis

| | |
|---|---|
| SEQ ID NO:5 | 2142 bp |

| | |
|---|---|
| NOV3a, | TATTATTCAGCAAACAATCTCAATGTGTTCCTGATGGGAGAGAGAGCATCTGGAAAAA |
| CG100760-01 | |
| DNA Sequence | CTATTGTTATAAATCTGGCTGTGTTGAGGTGGATCAAGGGTGAGATGTGGCAGAACAT |
| | GATCTCGTACGTCGTTCACCTCACTGCTCACGAAATAAACCAGATGACCAACAGCAGC |
| | TTGGCTGAGCTAATCGCCAAGGACTGGCCTGACGGCCAGGCTCCCATTGCAGACATCC |
| | TGTCTGATCCCAAGAAACTCCTTTTCATCCTCGAGGACTTGGACAACATAAGATTCGA |
| | GTTAAATGTCAATGAAAGTGCTTTGTGTAGTAACAGCACCCAGAAAGTTCCCATTCCA |
| | GTTCTCCTGGTCAGTTTGCTGAAGAGAAAAATGGCTCCAGGCTGCTGGTTCCTCATCT |
| | CCTCAAGGCCCACACGTGGGAATAATGTAAAACGTTCTTGAAAAGAGGTAGATTGCTG |
| | CACGACCTTGCAGCTGTCGAATGGGAAGAGGGAGATATATTTTTCTCTTTCTTTTAAA |
| | GACCGCCAGAGGGCGTCGGCAGCCCTCCAGCTTGTACATGAGGATGAAATACTCGTGG |
| | GTCTGTGCCGAGTCGCCATCTTATGCTGGATCACGTGTACTGTCCTGAAGCGGCAGAT |
| | GGACAAGGGGCGTGACTTCCAGCTCTGCTGCCAAACACCCACTGATCTACATGCCCAC |
| | TTTCTTGCTGATGCGTTGACATCAGAGGCTGGACTTACTGCCAATCAGTATCACCTAG |
| | GTCTCCTAAAACGTCTGTGTTTGCTGGCTGCAGGAGGACTGTTTCTGAGCACCCTGAA |
| | TTTCAGTGGTGAAGACCTCAGATGTGTTGGGTTTACTGAGGCTGATGTCTCTGTGTTG |
| | CAGGCCGCGAATATTCTTTTGCCGAGCAACACTCATAAAGACCGTTACAAGTTCATAC |
| | ACTTGAACGTCCAGGAGTTTTGTACAGCCATTGCATTTCTGATGGCAGTACCCAACTA |
| | TCTGATCCCCTCAGGCAGCAGAGAGTATAAAGAGAAGAGAGAACAATACTCTGACTTT |
| | AATCAAGTGTTTACTTTCATTTTTGGTCTTCTAAATGCAAACAGGAGAAAGATTCTTG |
| | AGACATCCTTTGGATACCAGCTACCGATGGTAGACAGCTTCAAGTGGTACTCGGTGGG |
| | ATACATGAACATTTGGACCGTGACCCGGAAAAGTTGACGCACCATATGCCTTTGTTT |
| | TACTGTCTCTATGAGAATCGGGAAGAAGAATTTGTGAAGACGATTGTGGATGCTCTCA |
| | TGGAGGTTACAGTTTACCTTCAATCAGACAAGGATATGATGGTCTCATTATACTGTCT |
| | GGATTACTGCTGTCACCTGAGGACACTTAAGTTGAGTGTTCAGCGCATCTTTCAAAAC |
| | AAACTGGAGAAATGCAACTTGTCGGCAGCCAGCTGTCAGGACCTAGCCTTGTTTCTCA |
| | CCAGCATCCAACACGTAACTCGATTGTGCCTGGGATTTAATCGGCTCCAAGATGATGG |
| | CATAAAGCTATTGTGTGCGGCCCTGACTCACCCCAAGTGTGCCTTAGAGAGACTGGAG |
| | CTCTGGTTTTGCCAGCTGGCAGCACCCGCTTGCAAGCACTTGTCAGATGCTCTCCTGC |
| | AGAACAGGAGCCTGACACACCTGAATCTGAGCAAGAACAGCCTGAGAGACGAGGGAGT |
| | CAAGTTCCTGTGTGAGGCCTTGGGTCGCCCAGATGGTAACCTGCAGAGCCTGAGTTTG |
| | TCAGGTTGTTCTTTCACAAGAGAGGGCTGTGGAGAGCTGGCTAATGCCCTCAGCCATA |
| | ATCATAATGTGAAAATCTTGGATTTGGGAGAAAATGATCTTCAGGATGATGGAGTGAA |
| | GCTACTGTGTGAGGCTCTGAAACCACATCGTGCATTGCACACACTTGGGTTGGCGAAA |
| | TGCAATCTGACAACTGCTTGCTGCCAGCATCTCTTCTCTGTTCTCAGCAGCAGTAAGA |
| | GCCTGGTCAATCTGAACCTTCTAGGCAATGAATTGGATACTGATGGTGTCAAGATGCT |
| | ATGTAAGGCTTTGAAAAAGTCGACATGCAGGCTGCAGAAACTCGGGTAAACCTCACTG |
| | ACTTTTCTGCAGGGGAGAACATACAGGGACAAGGCTAGATTGACTAGGCTTCTA |

| | | |
|---|---|---|
| ORF Start: ATG at 34 | | ORF Stop: TAA at 2077 |
| SEQ ID NO:6 | 681aa | MW at 76724.1 kD |

| | |
|---|---|
| NOV3a, | MGERASGKTIVINLAVLRWIKGEMWQNMISYVVHLTAHEINQMTNSSLAELIAKDWPD |
| CG100760-01 | |
| Protein Sequence | GQAPIADILSDPKKLLFILEDLDNIRFELNVNESALCSNSTQKVPIPVLLVSLLKRKM |
| | APGCWFLISSRPTRGNNVKTFLKEVDCCTTLQLSNGKREIYFNSFFKDRQRASAALQL |
| | VHEDEILVGLCRVAILCWITCTVLKRQMDKGRDFQLCCQTPTDLHAHFLADALTSEAG |
| | LTANQYHLGLLKRLCLLAAGGLFLSTLNFSGEDLRCVGFTEADVSVLQAANILLPSNT |
| | HKDRYKFIHLNVQEFCTAIAFLMAVPNYLIPSGSREYKEKREQYSDFNQVFTFIFGLL |
| | NANRRKILETSFGYQLPMVDSFKWYSVGYMKHLDRDPEKLTHHMPLFYCLYENREEEF |
| | VKTIVDALMEVTVYLQSDKDMMVSLYCLDYCCHLRTLKLSVQRIFQNKLEKCNLSAAS |
| | CQDLALFLTSIQHVTRLCLGFNRLQDDGIKLLCAALTHPKCALERLELWFCQLAAPAC |
| | KHLSDALLQNRSLTHLNLSKNSLRDEGVKFLCEALGRPDGNLQSLSLSGCSFTREGCG |
| | ELANALSHNHNVKILDLGENDLQDDGVKLLCEALKPHRALHTLGLAKCNLTTACCQHL |
| | FSVLSSSKSLVNLNLLGNELDTDGVKMLCKALKKSTCRLQKLG |

Further analysis of the NOV3a protein yielded the following properties shown in Table 3B.

TABLE 3B
Protein Sequence Properties NOV3a

| | |
|---|---|
| PSort | 0.8200 probability located in endoplasmic reticulum |
| analysis: | (membrane); 0.1900 probability located in plasma membrane; |
| | 0.1000 probability located in endoplasmic reticulum (lumen); |
| | 0.1000 probability located in outside |
| SignalP | Cleavage site between residues 23 and 24 |
| analysis: | |

A search of the NOV3a protein against the Geneseq database, a proprietary database that contains sequences published in patents and patent publication, yielded several homologous proteins shown in Table 3C.

TABLE 3C
Geneseq Results for NOV3a

| | | NOV3a | Identities/ | |
|---|---|---|---|---|
| | | Residues/ | Similarities for | |
| Geneseq | Protein/Organism/Length [Patent | Match | the Matched | Expect |
| Identifier | #, Date] | Residues | Region | Value |
| AAM50330 | Human nucleotide binding site | 1 . . . 681 | 539/745 (72%) | 0.0 |
| | protein NBS-3 - Homo sapiens, 875 | 116 . . . 859 | 587/745 (78%) | |
| | aa. [WO200183753-A2, 08-NOV-2001] | | | |
| AAM50326 | Human nucleotide binding site | 1 . . . 510 | 469/517 (90%) | 0.0 |
| | protein NBS-3 - Homo sapiens, 631 | 116 . . . 631 | 479/517 (91%) | |
| | aa. [WO200183753-A2, 08-NOV-2001] | | | |
| AAM50328 | Human nucleotide binding site | 2 . . . 681 | 247/750 (32%) | e−105 |
| | protein NBS-5 - Homo sapiens, 858 | 48 . . . 792 | 381/750 (49%) | |
| | aa. [WO200183753-A2, 08-NOV-2001] | | | |
| AAE07514 | Human PYRIN-1 protein - Homo | 2 . . . 680 | 238/729 (32%) | e−100 |
| | sapiens, 1034 aa. [WO200161005- | 224 . . . 944 | 362/729 (49%) | |
| | A2, 23-AUG-2001] | | | |
| ABG03924 | Novel human diagnostic protein | 2 . . . 680 | 228/741 (30%) | 7e−78 |
| | #3915 - Homo sapiens, 952 aa. | 178 . . . 908 | 334/741 (44%) | |
| | [WO200175067-A2, 11-OCT-2001] | | | |

In a BLAST search of public sequence databases, the NOV3a protein was found to have homology to the proteins shown in the BLASTP data in Table 3D.

TABLE 3D
Public BLASTP Results for NOV3a

| | | NOV3a | Identities/ | |
|---|---|---|---|---|
| Protein | | Residues/ | Similarities for | |
| Accession | | Match | the Matched | Expect |
| Number | Protein/Organism/Length | Residues | Portion | Value |
| CAD19388 | SEQUENCE 15 FROM PATENT | 1 . . . 681 | 539/745 (72%) | 0.0 |
| | WO0183753 - Homo sapiens | 116 . . . 859 | 587/745 (78%) | |
| | (Human), 875 aa. | | | |
| CAD19384 | SEQUENCE 3 FROM PATENT | 1 . . . 510 | 469/517 (90%) | 0.0 |
| | WO0183753 - Homo sapiens | 116 . . . 631 | 479/517 (91%) | |
| | (Human), 631 aa (fragment). | | | |
| CAD19386 | SEQUENCE 7 FROM PATENT | 2 . . . 681 | 247/750 (32%) | e−104 |
| | WO0183753 - Homo sapiens | 48 . . . 792 | 381/750 (49%) | |
| | (Human), 858 aa (fragment). | | | |
| Q96MN2 | CDNA FLJ32126 FIS, CLONE | 2 . . . 681 | 247/750 (32%) | e−104 |
| | PEBLM2000112, WEAKLY | 80 . . . 824 | 381/750 (49%) | |
| | SIMILAR TO Homo sapiens | | | |
| | NUCLEOTIDE-BINDING SITE | | | |
| | PROTEIN 1 MRNA - Homo sapiens | | | |
| | (Human), 919 aa. | | | |
| Q96MN2 | NACHT-, LRR- and PYD-containing | 2 . . . 681 | 247/750 (32%) | e−104 |
| | protein 4 (PAAD and NACHT- | 155 . . . 899 | 381/750 (49%) | |
| | containing protein 2) (PYRIN- | | | |
| | containing APAF1-like protein 4) | | | |
| | (Ribonuclease inhibitor 2) - Homo | | | |
| | sapiens (Human), 994 aa. | | | |

### Example 4

The NOV4 clone was analyzed, and the nucleotide and encoded polypeptide sequences are shown in Table 4A.

TABLE 4A
NOV4 Sequence Analysis

| | |
|---|---|
| SEQ ID NO:7 | 782 bp |

| | |
|---|---|
| NOV4a, | TCTCAAGGGATAATCACTAAATTCTGCCGAAAGGACTGAGGAACGGTGCCTGGAAAAG |
| CG100851-02 | |
| DNA Sequence | GGCAAGAATATCACGGCATGGGCATGAGTAGCTTGAAACTGCTGAAGTATGTCCTGTT |
| | TTTCTTCAACTTGCTCTTTTGGATCTGTGGCTGCTGCATTTTGGGCTTTGGGATCTAC |
| | CTGCTGATCCACAACAACTTCGGAGTGCTCTTCCATAACCTCCCCTCCCTCACGCTGG |
| | GCAATGTGTTTGTCATCGTGGGCTCTATCAAGGAAAACAAGTGTCTGCTTATGTCGTT |
| | CTTCATCCTGCTGCTGATTATCCTCCTTGCTGAGGTGACCTTGGCCATCCTGCTCTTT |
| | GTATATGAACAGAAGCTGAATGAGTATGTGGCTAAGGGTCTGACCGACAGCATCCACC |
| | GTTACCACTCAGACAATAGCACCAAGGCAGCGTGGGACTCCATCCAGTCATTTCTGCA |
| | GTGTTGTGGTATAAATGGCACGAGTGATGGGACCAGTGGCCCACCAGCATCTTGCCCC |
| | TCAGATCGAAAAGTGGAGGGTTGCTATGCGAAAGCAAGACTGTGGTTTCATTCCAATT |
| | TCCTGTATATCGGAATCATCACCATCTGTGTATGTGTGATTGAGGTGTTGGGGATGTC |
| | CTTTGCACTGACCCTGAACTGCCAGATTGACAAAACCAGCCAGACCATAGGGCTATGA |
| | TCTGCAGTAGTTCTGTGGTGAAGAGACTTGTTTCATCTCCTGGAAATGCAAAACCATT |
| | TATAGCATGAGCCCTACATGATCATCAG |

| | | |
|---|---|---|
| ORF Start: ATG at 76 | | ORF Stop: TGA at 694 |
| SEQ ID NO:8 | 206aa | MW at 22888.8 kD |

| | |
|---|---|
| NOV4a, | MGMSSLKLLKYVLFFFNLLFWICGCCILGFGIYLLIHNNFGVLFHNLPSLTLGNVFVI |
| CG100851-02 | |
| Protein Sequence | VGSIKENKCLLMSFFILLLIILLAEVTLAILLFVYEQKLNEYVAKGLTDSIHRYHSDN |
| | STKAAWDSIQSFLQCCGINGTSDGTSGPPASCPSDRKVEGCYAKARLWFHSNFLYIGI |
| | ITICVCVIEVLGMSFALTLNCQIDKTSQTIGL |

Further analysis of the NOV4a protein yielded the following properties shown in Table 4B.

TABLE 4B
Protein Sequence Properties NOV4a

| | |
|---|---|
| PSort | 0.6400 probability located in plasma membrane; |
| analysis: | 0.4600 probability located in Golgi body; |
| | 0.3700 probability located in endoplasmic |
| | reticulum (membrane); 0.1000 probability located |
| | in endoplasmic reticulum (lumen) |
| SignalP | Cleavage site between residues 32 and 33 |
| analysis: | |

A search of the NOV4a protein against the Geneseq database, a proprietary database that contains sequences published in patents and patent publication, yielded several homologous proteins shown in Table 4C.

TABLE 4C
Geneseq Results for NOV4a

| | | NOV4a | Identities/ | |
|---|---|---|---|---|
| | | Residues/ | Similarities for | |
| Geneseq | Protein/Organism/Length [Patent | Match | the Matched | Expect |
| Identifier | #, Date] | Residues | Region | Value |
| AAY96141 | Human haematopoietic CD53 - | 1 . . . 206 | 205/219 (93%) | e−115 |
| | Homo sapiens, 219 aa. | 1 . . . 219 | 205/219 (93%) | |
| | [US6111093-A, 29-AUG-2000] | | | |
| AAB58136 | Lung cancer associated polypeptide | 1 . . . 206 | 205/219 (93%) | e−115 |
| | sequence SEQ ID 474 - Homo | 13 . . . 231 | 205/219 (93%) | |
| | sapiens, 231 aa. [WO200055180- | | | |
| | A2, 21-SEP-2000] | | | |
| AAW89152 | Human CD53 antigen - Homo | 1 . . . 206 | 205/219 (93%) | e−115 |
| | sapiens, 219 aa. [US5849898-A, 15-DEC-1998] | 1 . . . 219 | 205/219 (93%) | |
| AAW80455 | Human CD53 antigen - Homo | 1 . . . 206 | 205/219 (93%) | e−115 |
| | sapiens, 219 aa. [US5830731-A, 03-NOV-1998] | 1 . . . 219 | 205/219 (93%) | |
| AAR91446 | Human CD53 antigen - Homo | 1 . . . 206 | 205/219 (93%) | e−115 |
| | sapiens, 219 aa. [US5506126-A, 09-APR-1996] | 1 . . . 219 | 205/219 (93%) | |

In a BLAST search of public sequence databases, the NOV4a protein was found to have homology to the proteins shown in the BLASTP data in Table 4D.

TABLE 4D
Public BLASTP Results for NOV4a

| | | NOV4a | Identities/ | |
|---|---|---|---|---|
| Protein | | Residues/ | Similarities for | |
| Accession | | Match | the Matched | Expect |
| Number | Protein/Organism/Length | Residues | Portion | Value |
| P19397 | Leukocyte surface antigen CD53 | 1 . . . 206 | 205/219 (93%) | e−115 |
| | (Cell surface glycoprotein CD53) - | 1 . . . 219 | 205/219 (93%) | |
| | Homo sapiens (Human), 219 aa. | | | |
| AAH21310 | CD53 ANTIGEN - Mus musculus | 1 . . . 206 | 168/219 (76%) | 6e−95 |
| | (Mouse), 219 aa. | 1 . . . 219 | 183/219 (82%) | |
| Q61451 | Leukocyte surface antigen CD53 | 2 . . . 206 | 167/218 (76%) | 2e−94 |
| | (Cell surface glycoprotein CD53) - | 1 . . . 218 | 182/218 (82%) | |
| | Mus musculus (Mouse), 218 aa. | | | |
| A39574 | leukocyte antigen OX-44 - rat, 219 | 1 . . . 206 | 164/219 (74%) | 7e−94 |
| | aa. | 1 . . . 219 | 183/219 (82%) | |
| P24485 | Leukocyte surface antigen CD53 | 2 . . . 206 | 163/218 (74%) | 3e−93 |
| | (Cell surface glycoprotein CD53) | 1 . . . 218 | 182/218 (82%) | |
| | (Leukocyte antigen MRC OX-44) - | | | |
| | Rattus norvegicus (Rat), 218 aa. | | | |

PFam analysis predicts that the NOV4a protein contains the domains shown in the Table 4E.

TABLE 4E
Domain Analysis of NOV4a

| | | Identities/ | |
|---|---|---|---|
| | NOV4a | Similarities | Expect |
| Pfam Domain | Match Region | for the Matched Region | Value |
| transmembrane4 | 10 . . . 36 | 17/27 (63%) | 4.4e−08 |
| | | 27/27 (100%) | |
| transmembrane4 | 58 . . . 197 | 52/202 (26%) | 1.2e−44 |
| | | 120/202 (59%) | |

### Example 5

The NOV5 clone was analyzed, and the nucleotide and encoded polypeptide sequences are shown in Table 5A.

TABLE 5A
NOV5 Sequence Analysis

| | |
|---|---|
| SEQ ID NO:9 | 1719 bp |

| | |
|---|---|
| NOV5a, | ATGCGGACGCCGGTGGTGATGACGCTGGGCATGGTGTTGGCGCCCTGCGGGCTCCTGC |
| CG101068-01 | |
| DNA Sequence | TCAACCTGACCGGCACCCTGGCGCCCGGCTGGCGGCTGGTGAAGGGCTTCCTGAACCA |
| | GCCAGTGGACGTGGAGTTGTACCAGGGCCTGTGGGACATGTGTCGCGAGCAGAGCAGC |
| | CGCGAGCGCGAGTGCGGCCAGACGGACCAGTGGGGCTACTTCGAGGCCCAGCCCGTGC |
| | TGGTGGCGCGGGCACTCATGGTCACCTCGCTGGCCGCCACGGTCCTGGGGCTTCTGCT |
| | GGCGTCGCTGGGCGTGCGCTGCTGGCAGGACGAGCCCAACTTCGTGCTGGCAGGGCTC |
| | TCGGGCGTCGTGCTCTTCGTCGCTGGCCTCCTCGGCCTCATCCCGGTGTCCTGGTACA |
| | ACCACTTCTTGGGGGACCGCGACGTGCTGCCCGCCCCGGCCAGCCCGGTCACGGTGCA |
| | GGTCAGCTACAGCCTGGTCCTGGGCTACCTGGGCAGCTGCCTCCTGCTGCTGGGCGGC |
| | TTCTCGCTGGCGCTCAGCTTCGCGCCCTGGTGCGACGAGCGTTGTCGCCGCCGCCGCA |
| | AGGGACCCTCCGCCGGGCCTCGCCGCAGCAGCGTCAGCACCATCCAAGTGGAGTGGCC |
| | CGAGCCCGACCTGGCGCCCGCCATCAAGTACTACAGCGACGGCCAGCACCGACCGCCG |
| | CCTGCCCAGCACCGCAAGCCCAAGCCCAAGCCCAAGGTCGGCTTCCCCATGCCGCGGC |
| | CGCGGCCCAAGGCCTACACCAACTCGGTGGACGTCCTCGACGGGGAGGGGTGGGAGTC |
| | CCAGGACGCTCCCTCGTGCAGCACCCACCCCTGCGACAGCTCGCTGCCCTGCGACTCC |
| | GACCTCTAGACGCTTGTAGAGCCTGGGGGGCGCCGGGTGGCAAAGGACTCACCCCCGC |
| | ACAGGCCCGCCTGGCTTCGAGTTGGAACCCGGACACTTGCCCCTCACTGGTGTGGATG |
| | GAAATCTGCCTTTCGTGGGACCAAACAGGACTCCTTGGACGATTAGTTCAGGTTGGGT |
| | TTGGTTTTCTTCTTAAAGAGTTTAGTTTTCCTCTCCAGAGGGATCAGGGTCCTCTTAG |
| | GGAGTGACCGGCTTTTCATATATTTTTGCTGAAGAATATATGGAAAGGGTGCCATTTG |
| | CGTCACGTGGACCAGGGACAGTGCTGAAATCAGCAGTGCTCAGAAACAATTTAACATG |
| | TTGAAACGACAATATTCTAAAATACTGATGAATCTTGCATCAATATAATTATTGGGTT |
| | TTTTTTCTTTTTCCTGCTGTATAACTCCTTGCCATGCAAACTCTCAAGAGGCCAATAT |
| | ATTCCTGGCCATGTTTGAATGAGCCTCTTAAAATAAACTTAGAGCCATGCAAATGCCA |
| | GCAGCTTAATGGATTTCATGGAATGAAATACCGTGATTAACTCATAGCTACATATCAT |
| | TGCATAAATQGGATTTATCTTTTTTCTCACTTATTTTTGCGGTGAAAGTCGAGGGCAT |
| | GCAAGAGTTTCTCTTCCAGAAGCCAAGAGGAGAACAAAGGTCCTAATGCTGTACTATT |
| | CCACCCTTTGGACGCCTCATCCAGGACGCAGAGGACTCTAGGTTTAACATTTTGTACA |
| | AAATGGAACCTGTTAATCATATTAAAGCACATATGTATATATCTTTTATTTATAAATA |
| | AAATTTTAAAACAATAGTTTCAGTATAGCCACAAAAA |

| | | |
|---|---|---|
| ORF Start: ATG at 1 | | ORF Stop: TAG at 877 |
| SEQ ID NO:10 | 292 aa | MW at 31914.5 kD |

| | |
|---|---|
| NOV5a, | MRTPVVMTLGMVLAPCGLLLNLTGTLAPGWRLVKGFLNQPVDVELYQGLWDMCREQSS |
| CG101068-01 | RERECGQTDQWGYFEAQPVLVAPALMVTSLAATVLGLLLASLGVRCWQDEPNFVLAGL |
| Protein Sequence | |
| | SGVVLFVAGLLGLIPVSWYNHFLGDRDVLPAPASPVTVQVSYSLVLGYLGSCLLLLGG |
| | FSLALSFAPWCDERCRRRRKGPSAGPRRSSVSTIQVEWPEPDLAPAIKYYSDGQHRPP |
| | PAQHRKPKPKPKVGFPMPRPRPKAYTNSVDVLDGEGWESQDAPSCSTHPCDSSLPCDS |
| | DL |

Further analysis of the NOV5a protein yielded the following properties shown in Table 5B.

TABLE 5B
Protein Sequence Properties NOV5a

| | |
|---|---|
| PSort | 0.6400 probability located in plasma membrane; |
| analysis: | 0.4600 probability located in Golgi body; 0.3700 probability |
| | located in endoplasmic reticulum (membrane); |
| | 0.1000 probability located in endoplasmic reticulum (lumen) |
| SignalP | Cleavage site between residues 28 and 29 |
| analysis: | |

A search of the NOV5a protein against the Geneseq database, a proprietary database that contains sequences published in patents and patent publication, yielded several homologous proteins shown in Table 5C.

TABLE 5C
Geneseq Results for NOV5a

| | | NOV5a | Identities/ | |
|---|---|---|---|---|
| | | Residues/ | Similarities for | |
| Geneseq | Protein/Organism/Length [Patent | Match | the Matched | Expect |
| Identifier | #, Date] | Residues | Region | Value |
| AAB64401 | Amino acid sequence of human | 9 . . . 206 | 74/206 (35%) | 1e−16 |
| | intracellular signalling molecule | 9 . . . 209 | 101/206 (48%) | |
| | INTRA33 - Homo sapiens, 217 aa. | | | |
| | [WO200077040-A2, 21-DEC-2000] | | | |
| AAG75467 | Human colon cancer antigen protein | 6 . . . 187 | 59/188 (31%) | 2e−13 |
| | SEQ ID NO: 6231 - Homo sapiens, | 7 . . . 192 | 92/188 (48%) | |
| | 210 aa. [WO200122920-A2, 05-APR-2001] | | | |
| ABB50278 | Claudin 4 ovarian tumor marker | 6 . . . 187 | 59/188 (31%) | 2e−13 |
| | protein, SEQ ID NO: 45 - Homo | 6 . . . 191 | 92/188 (48%) | |
| | sapiens, 209 aa. [WO200175177-A2, | | | |
| | 11-OCT-2001] | | | |
| AAB43133 | Human ORFX ORF2897 | 6 . . . 187 | 59/188 (31%) | 2e−13 |
| | polypeptide sequence SEQ ID | 6 . . . 191 | 92/188 (48%) | |
| | NO: 5794 - Homo sapiens, 209 aa. | | | |
| | [WO200058473-A2, 05-OCT-2000] | | | |
| ABB50396 | Human secreted protein encoded by | 9 . . . 187 | 59/185 (31%) | 2e−13 |
| | gene 96 SEQ ID NO: 344 - Homo | 1 . . . 183 | 91/185 (48%) | |
| | sapiens, 202 aa. [WO200162891-A2, 30-AUG-2001] | | | |

In a BLAST search of public sequence databases, the NOV5a protein was found to have homology to the proteins shown in the BLASTP data in Table 5D.

TABLE 5D
Public BLASTP Results for NOV5a

| | | NOV5a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q96B33 | SIMILAR TO RIKEN CDNA | 28 . . . 292 | 252/267 (94%) | e−147 |
| | 2310014B08 GENE - Homo | 2 . . . 268 | 254/267 (94%) | |
| | sapiens (Human), 268 aa | | | |
| | (fragment). | | | |
| Q9D7D7 | 2310014B08RIK PROTEIN | 1 . . . 292 | 230/296 (77%) | e−135 |
| | (RIKEN CDNA 2310014B08 | 1 . . . 296 | 248/296 (83%) | |
| | GENE) - Mus musculus (Mouse), | | | |
| | 296 aa. | | | |
| O95484 | Claudin-9 - Homo sapiens | 9 . . . 206 | 74/206 (35%) | 4e−16 |
| | (Human), 217 aa. | 9 . . . 209 | 101/206 (48%) | |
| Q9Z0S7 | Claudin-9 - Mus musculus | 9 . . . 206 | 71/206 (34%) | 1e−14 |
| | (Mouse), 217 aa. | 9 . . . 209 | 99/206 (47%) | |
| Q98SR2 | CLAUDIN-3 - Gallus gallus | 10 . . . 206 | 64/202 (31%) | 1e−13 |
| | (Chicken), 214 aa. | 9 . . . 207 | 99/202 (48%) | |

PFam analysis predicts that the NOV5a protein contains the domains shown in the Table 5E.

TABLE 5E
Domain Analysis of NOV5a

| | | Identities/ | |
|---|---|---|---|
| | NOV5a | Similarities | Expect |
| Pfam Domain | Match Region | for the Matched Region | Value |
| PMP22_Claudin | 3 . . . 177 | 40/194 (21%) | 0.00018 |
| | | 108/194 (56%) | |

### Example 6

The NOV6 clone was analyzed, and the nucleotide and encoded polypeptide sequences are shown in Table 6A.

TABLE 6A
NOV6 Sequence Analysis

| | |
|---|---|
| SEQ ID NO:11 | 2369 bp |

| | |
|---|---|
| NOV6a, | CGGCCGGAGCGCCGAGGCCCGGCCATGGCCACCACCAGCACCACGGGCTCCACCCTGC |
| CG101231-01 | |
| DNA Sequence | TGCAGCCCCTCAGCAACGCCGTGCAGCTGCCCATCGACCAGGTCAACTTTGTAGTGTG |
| | CCAACTCTTTGCCTTGCTAGCAGCCATTTGGTTTCGAACTTATCTACATTCAAGCAAA |
| | ACTAGCTCTTTTATAAGACATGTAGTTGCTACCCTTTTGGGCCTTTATCTTGCACTTT |
| | TTTGCTTTGGATGGTATGCCTTACACTTTCTTGTACAAAGTGGAATTTCCTACTGTAT |
| | CATGATCATCATAGGAGTGGAGAACATGCACAATTACTGCTTTGTGTTTGCTCTGGGA |
| | TACCTCACAGTGTGCCAAGTTACTCGAGTCTATATCTTTGACTATGGACAATATTCTG |
| | CTGATTTTTCAGGCCCAATGATGATCATTACTCAGAAGATCACTAGTTTGGCTTGCGA |
| | AATTCATGATGGGATGTTTCGGAAGGATGAAGAACTGACTTCCTCACAGAGGGATTTA |
| | GCTGTAAGGCGCATGCCAAGCTTACTGGAGTATTTGAGTTACAACTGTAACTTCATGG |
| | GGATCCTGGCAGGCCCACTTTGCTCTTACAAAGACTACATTACTTTCATTGAAGGCAG |
| | ATCATACCATATCACACAATCTGGTGAAAATGGAAAAGAAGAGACACAGTATGAAAGA |
| | ACAGAGCCATCTCCAAATAGTGCGGTTGTTCAGAAGCTCTTAGTTTGTGGGCTGTCCT |
| | TGTTATTTCACTTGACCATCTGTACAACATTACCTGTGGAGTACAACATTGATGAGCA |
| | TTTTCAAGCTACAGCTTCGTGGCCAACAAAGATTATCTATCTGTATATCTCTCTTTTG |
| | GCTGCCAGACCCAATACTATTTTGCATGGACGCTAGCTGATGCCATTAATAAATGCTG |
| | CAGGCTTTGGTTTCAGAGGGTATGACGAAAATGGAGCAGCTCGCTGGGACTTAATTTC |
| | CAATTTGAGAATTCAACAATAGAGATGTCAACAAGTTTCAAGATGTTTCTTGATAAAT |
| | TGGAATATTCAGACAGCTCTTTGGCTCAAAAGGGTGTGTTATGAACGAACCTCCTTCA |
| | GTCCAACTATCCAGACGTTCATTCTCTCTGCCATTTGGCACGGGGTATACCCAGGATA |
| | TTATCTAACGTTTCTAACAGGGGTGTTAATGACATTAGCAGCAAGAGCTGTAAGAAAT |
| | AACTTTAGACATTATTTCATTGAACCTTCCCAACTGAAATTATTTTATGATGTTATAA |
| | CATGGATAGTAACTCAAGTAGCAATAAGTTACACACTTGTGCCATTTGTGCTTCTTTC |
| | TATAAAACCATCACTCACGTTTTACAGCTCCTGGTATTATTGCCTGCACATTCTTGGT |
| | ATCTTAGTATTATTGTTGTTGCCAGTAAAAAAAACTCAAAGAAGAAAGAATACACATG |
| | AAAACATTCAGCTCTCACAATCCAAAAAGTTTGATGAAGGAGAAAATTCTTTGGGACA |
| | GAACAGTTTTTCTACAACAAACAATGTTTGCAATCAGAATCAAGAAATAGCCTCGAGA |
| | CATTCATCACTAAAGCAGTGATCGGGAAGGCTCTGAGGGCTGTTTTTTTTTTTTGATG |
| | TTAACAGAAACCAATCTTAGCACCTTTTCAAGGGGTTTGAGTTTGTTGGAAAAGCAGT |
| | TAACTGGGGGGAAATGGACAGTTATAGATAAGGAATTTCCTGTACACCAGATTCGAAA |
| | TGGAGTGAAACAAGCCCTCCCATGCCATGTCCCCGTGGGCCACGCCTTATGTAAGAAT |
| | ATTTCCATATTTCAGTGGGCACTCCCAACCTCAGCACTTGTCCGTAGGGTCACACGCG |
| | TGCCCTGTTGCTGAATGTATGTTGCGTATCCCAAGGCACTGAAGAGGTGGAAAAATAA |
| | TCGTGTCAATCTGGATGATAGAGAGAAATTAACTTTTCCAAATGAATGTCTTGCCTTA |
| | AACCCTCTATTTCCTAAAATATTGTTCCTAAATGGTATTTTCAAGTGTAATATTGTGA |
| | GAACGCTACTGCAGTAGTTGATGTTGTGTGCTGTAAAGGATTTTAGGAGGAATTTGAA |
| | ACAGGATATTTAAGAGTGTGGATATTTTTAAAATGCAATAAACATCTCAGTATTTGAA |
| | GGGTTTTCTTAAAGTATGTCAAATGACTACAATCCATAGTGAAACTGTAAACAGTAAT |
| | GGACGCCAAATTATAGGTAGCTGATTTTGCTGGAGAGTTTAATTACCTTGTGCAGTCA |
| | AAGAGCGCTTCCAGAAGGAATCTCTTAAAACATAATGAGAGGTTTGGTAATGTGATAT |
| | TTTAAGCTTACTCTTTTTCTTAAAAGAGAGAGGTGACGAAGGAAGGCAG |

| | | |
|---|---|---|
| ORF Start: ATG at 25 | | ORF Stop: TGA at 1585 |
| SEQ ID NO:12 | 520 aa | MW at 59480.0 kD |

| | |
|---|---|
| NOV6a, | MATTSTTGSTLLQPLSNAVQLPIDQVNFVVCQLFALLAAIWFRTYLHSSKTSSFIRHV |
| CG101231-01 | |
| Protein Sequence | VATLLGLYLALFCFGWYALHFLVQSGISYCIMIIIGVENNHNYCFVFALGYLTVCQVT |
| | RVYIFDYGQYSADFSGPMMIITQKITSLACEIHDGMFRKDEELTSSQRDLAVRRMPSL |
| | LEYLSYNCNFMGILAGPLCSYKDYITFIEGRSYHITQSGENGKEETQYERTEPSPNSA |
| | VVQKLLVCGLSLLFHLTICTTLPVEYNIDEHFQATASWPTKIIYLYISLLAARPKYYF |
| | AWTLADAINNAAGFGFRGYDENGAARWDLISNLRIQQIEMSTSFKMFLDNWNIQTALW |
| | LKRVCYERTSFSPTIQTFILSAIWHGVYPGYYLTFLTGVLMTLAARAVRNNFRHYFIE |
| | PSQLKLFYDVITWIVTQVAISYTVVPFVLLSIKPSLTFYSSWYYCLHILGILVLLLLP |
| | VKKTQRRKNTHENIQLSQSKKFDEGENSLGQNSFSTTNNVCNQNQEIASRHSSLKQ |

| | |
|---|---|
| SEQ ID NO:13 | 2270 bp |

| | |
|---|---|
| NOV6b, | CGGCCGGAGCGCCGAGGCCCGGCCATGGCCACCACCAGCACCACGGGCTCCACCCTGC |
| CG101231-02 | |
| DNA Sequence | TGCAGCCCCTCAGCAACGCCGTGCAGCTGCCCATCGACCAGGTCAACTTTGTAGTGTG |
| | CCAACTCTTTGCCTTGCTAGCAGCCATTTGGTTTCGAACTTATCTACATTCAAGCAAA |
| | ACTAGCTCTTTTATAAGACATGTAGTTGCTACCCTTTTGGGCCTTTATCTTGCACTTT |
| | TTTGCTTTGGATGGTATGCCTTACACTTTCTTGTACAAAGTGGAATTTCCTACTGTAT |
| | CATGATCATCATAGGAGTGGAGAACATGCAGCCAATGATGATCATTACTCAGAAGATC |
| | ACTAGTTTGGCTTGCGAAATTCATGATGGGATGTTTCGGAAGGATGAAGAACTGACTT |
| | CCTCACAGAGGGATTTAGCTGTAAGGCGCATGCCAAGCTTACTGGAGTATTTGAGTTA |
| | CAACTGTAACTTCATGGGGATCCTGGCAGGCCCACTTTGCTCTTACAAAGACTACATT |
| | ACTTTCATTGAAGGCAGATCATACCATATCACACAATCTGGTGAATGGAAAAGAAAAG |
| | AGACACAGTATGAAAGAACAGAGCCATCTCCAAATAGTGCGGTTGTTCAGAAGCTCTT |
| | AGTTTGTGGGCTGTCCTTGTTATTTCACTTGACCATCTGTACAACATTACCTGTGGAG |
| | TACAACATTGATGAGCATTTTCAAGCTACAGCTTCGTGGCCAACAAAGATTATCTATC |
| | TGTATATCTCTCTTTTGGCTGCCAGACCCAAATACTATTTTGCATGGACGCTAGCTGA |
| | TGCCATTAATAATGCTGCAGGCTTTGGTTTCAGAGGGTATGACGAAAATGGAGCAGCT |
| | CGCTGGGACTTAATTTCCAATTTGAGAATTCAACAATAGAGATGTCAACAAAGTTTCA |
| | AGATGTTTCTTGATAATTGGAATATTCAGACAGCTCTTTGGCTCAAAAGGGTCTGTTA |
| | TGAACGAACCTCCTTCAGTCCAACTATCCAGACGTTCATTCTCTCTGCCATTTGGCAC |
| | GGGGTATACCCAGGATATTATCTAACGTTTCTAACAGGGGTGTTAATGACATTAGCAG |
| | CAAGAGCTGTAAGAAATAACTTTAGACATTATTTCATTGAACCTTCCCAACTGAAATT |
| | ATTTTATGATGTTATAACATGGATAGTAACTCAAGTAGCAATAAGTTACACAGTTGTG |
| | CCATTTGTGCTTCTTTCTATAAAACCATCACTCACGTTTTACAGCTCCTGGTATTATT |
| | GCCTGCACATTCTTGGTATCTTAGTATTATTGTTGTTGCCAGTAAAAAAAACTCAAAG |
| | AAGAAAGAATACACATGAAAACATTCAGCTCTCACAATCCAAAAAGTTTGATGAAGGA |
| | GAAATTCTTTGGGACAGAACAGTTTTTCTACAACAAACAATGTTTGCAAATCAGAATC |
| | AAGAAATAGCCTCGAGACATTCATCACTAAAGCAGTGATCGGGAAGGCTCTGAGGGCT |
| | GTTTTTTTTTTTTGATGTTAACAGAAACCAATCTTAGCACCTTTTCAAGGGGTTTGAG |
| | TTTGTTGGAAAAGCAGTTAACTGGGGGGAAATGGACAGTTATAGATAAGGAATTTCCT |
| | GTACACCAGATTGGAAATGGAGTGAAACAAGCCCTCCCATGCCATGTCCCCGTGGGCC |
| | ACGCCTTATGTAAGAATATTTCCATATTTCAGTGGGCACTCCCAACCTCAGCACTTGT |
| | CCGTAGGGTCACACGCGTGCCCTGTTGCTGAATGTATGTTGCGTATCCCAAGGCACTG |
| | AAGAGGTGGAAAATAATCGTGTCAATCTGGATGATAGAGAGAAATTAACTTTTCCAAA |
| | ATGAATGTCTTGCCTTAAACCCTCTATTTCCTAAAATATTGTTCCTA3ATGGTATTTT |
| | CAAGTGTAATATTGTGAGAACGCTACTGCAGTAGTTGATGTTGTGTGCTGTAAAGGAT |
| | TTTAGGAGGAATTTGAAACAGGATATTTAAGAGTGTGGATATTTTTAAAATGCAATAA |
| | ACATCTCAGTATTTGAAGGGTTTTCTTAAAGTATGTCAAATGACTACAATCCATAGTG |
| | AAACTGTAAACAGTAATGGACGCCAAATTATAGGTAGCTGATTTTGCTGGAGAGTTTA |
| | ATTACCTTGTGCAGTCAAAGAGCGCTTCCAGAAGGAATCTCTTAAAACATAATGAGAG |
| | GTTTGGTAATGTGATATTTTAAGCTTACTCTTTTTCTTAAAAGAGAGAGGTGACGAAG |
| | GAAGGCAG |

| | | |
|---|---|---|
| ORF Start: ATG at 25 | | ORF Stop: TGA at 1486 |
| SEQ ID NO:14 | 487 aa | MW at 55677.7 kD |

| | |
|---|---|
| NOV6b, | MATTSTTGSTLLQPLSNAVQLPIDQVNFVVCQLFALLAAIWFRTYLHSSKTSSFIRHV |
| CG101231-02 | |
| Protein Sequence | VATLLGLYLALFCFGWYALHFLVQSGISYCIMIIIGVENMQPMMIITQKITSLACEIH |
| | DGMFRKDEELTSSQRDLAVRRMPSLLEYLSYNCNFMGILAGPLCSYKDYITFIEGRSY |
| | HITQSGENGKEETQYERTEPSPNSAVVQKLLVCGLSLLFHLTICTTLPVEYNIDEHFQ |
| | ATASWPTKIIYLYISLLAARPKYYFAWTLALAINNAAGFGFRGYDENGAAAWDLISNL |
| | RIQQIEMSTSFKMFLDNANIQTALWLKRVCYERTSFSPTIQTFILSAIWHGVYPGYYL |
| | TFLTGVLMTLAARAVRNNFRHYFIEPSQLKLFYDVITWIVTQVAISYTVVPFVLLSIK |
| | PSLTFYSSWYYCLHILGILVLLLLPVKKTQRRKNTHENIQLSQSKKFDEGENSLGQNS |
| | FSTTNNVCNQNQEASRHSSLKQ |

Sequence comparison of the above protein sequences yields the following sequence relationships shown in Table 6B.

TABLE 6B
Comparison of NOV6a against NOV6b.

| | NOV6a Residues/ | Identities/Similarities |
|---|---|---|
| Protein Sequence | Match Residues | for the Matched Region |
| NOV6b | 1 . . . 520 | 474/520 (91%) |
| | 1 . . . 487 | 474/520 (91%) |

Further analysis of the NOV6a protein yielded the following properties shown in Table 6C.

TABLE 6C
Protein Sequence Properties NOV6a

| | |
|---|---|
| PSort | 0.6000 probability located in plasma membrane; |
| analysis: | 0.4000 probability located in Golgi body; |
| | 0.3406 probability located in mitochondrial |
| | intermembrane space; |
| | 0.3384 probability located in mitochondrial inner membrane |
| SignalP | Cleavage site between residues 44 and 45 |
| analysis: | |

A search of the NOV6a protein against the Geneseq database, a proprietary database that contains sequences published in patents and patent publication, yielded several homologous proteins shown in Table 6D.

TABLE 6D
Geneseq Results for NOV6a

| | | NOV6a | Identities/ | |
|---|---|---|---|---|
| | | Residues/ | Similarities for | |
| Geneseq | Protein/Organism/Length [Patent | Match | the Matched | Expect |
| Identifier | #, Date] | Residues | Region | Value |
| AAG81345 | Human AFP protein sequence SEQ | 98 . . . 520 | 419/423 (99%) | 0.0 |
| | ID NO: 208 - Homo sapiens, 423 aa. | 1 . . . 423 | 421/423 (99%) | |
| | [WO200129221-A2, 26-APR-2001] | | | |
| AAB93797 | Human protein sequence SEQ ID | 102 . . . 520 | 416/419 (99%) | 0.0 |
| | NO: 13560 - Homo sapiens, 432 aa. | 14 . . . 432 | 419/419 (99%) | |
| | [EP1074617-A2, 07-FEB-2001] | | | |
| AAM93974 | Human stomach cancer expressed | 102 . . . 520 | 416/419 (99%) | 0.0 |
| | polypeptide SEQ ID NO: 17 - Homo | 14 . . . 432 | 419/419 (99%) | |
| | sapiens, 432 aa. [WO200109317- | | | |
| | A1, 08-FEB-2001] | | | |
| ABG04835 | Novel human diagnostic protein | 50 . . . 297 | 243/248 (97%) | e−143 |
| | #4826 - Homo sapiens, 371 aa. | 23 . . . 270 | 246/248 (98%) | |
| | [WO200175067-A2, 11-OCT-2001] | | | |
| ABG04835 | Novel human diagnostic protein | 50 . . . 297 | 243/248 (97%) | e−143 |
| | #4826 - Homo sapiens, 371 aa. | 23 . . . 270 | 246/248 (98%) | |
| | [WO200175067-A2, 11-OCT-2001] | | | |

In a BLAST search of public sequence databases, the NOV6a protein was found to have homology to the proteins shown in the BLASTP data in Table 6E.

TABLE 6E
Public BLASTP Results for NOV6a

| | | NOV6a | Identities/ | |
|---|---|---|---|---|
| Protein | | Residues/ | Similarities for | |
| Accession | | Match | the Matched | Expect |
| Number | Protein/Organism/Length | Residues | Portion | Value |
| AAH25429 | SIMILAR TO RIKEN CDNA | 1 . . . 520 | 451/520 (86%) | 0.0 |
| | 2810049G06 GENE - Mus | 1 . . . 519 | 479/520 (91%) | |
| | Musculus (Mouse), 519 aa. | | | |
| CAC38595 | SEQUENCE 207 FROM | 98 . . . 520 | 419/423 (99%) | 0.0 |
| | PATENT WO0129221 - Homo | 1 . . . 423 | 421/423 (99%) | |
| | sapiens (Human), 423 aa. | | | |
| AAH25020 | RIKEN CDNA 2810049G06 | 1 . . . 520 | 422/520 (81%) | 0.0 |
| | GENE - Mus musculus (Mouse), | 1 . . . 487 | 449/520 (86%) | |
| | 487 aa. | | | |
| Q9CZ73 | 2810049G06RIK PROTEIN - | 1 . . . 520 | 421/520 (80%) | 0.0 |
| | Mus musculus (Mouse), 487 aa. | 1 . . . 487 | 448/520 (85%) | |
| Q96KY4 | SIMILAR TO RIKEN CDNA | 171 . . . 520 | 348/350 (99%) | 0.0 |
| | 2810049G06 GENE - Homo | 1 . . . 350 | 350/350 (99%) | |
| | sapiens (Human), 350 aa. | | | |

PFam analysis predicts that the NOV6a protein contains the domains shown in the Table 6F.

TABLE 6F
Domain Analysis of NOV6a

| | | Identities/ | |
|---|---|---|---|
| | NOV6a | Similarities | Expect |
| Pfam Domain | Match Region | for the Matched Region | Value |
| Adeno_Penton_B | 204 . . . 222 | 8/20 (40%) | 0.54 |
| | | 17/20 (85%) | |
| MBOAT | 148 . . . 442 | 108/334 (32%) | 4.1e−89 |
| | | 225/334 (67%) | |

### Example 7

The NOV7 clone was analyzed, and the nucleotide and encoded polypeptide sequences are shown in Table 7A.

TABLE 7A
NOV7 Sequence Analysis

| | |
|---|---|
| SEQ ID NO:15 | 537 bp |

| | |
|---|---|
| NOV7a, | ATTAGCAACGGCTCATGATGAACTCAATCAAAGGGGGCTTGACCAGCATCTCAGGTCT |
| CG101362-01 | |
| DNA Sequence | ACTCTATGTTTTCCAGTGCCCATCAGTGCCAAGGGTATTGATTAGCCTATCCGGGACA |
| | AAGAGAGAAGAAAGAGTGAGACACCACCACTAAAAGGGCTGCAGGTGGATACCGCCTC |
| | CCTCAAGCTGGAAAAAGATTAGAAAGATGGTGAAAACAGGAAGACCTTCCTCATCCCA |
| | CTATCAGGAAGATGAGGAAAGAGATCAGGAGGATCACAGGTGGAGAGGAGAAGAGGAC |
| | CATGCTCGATCCTCTCTGGTAATAGGCCTGAGATTCCCTCTCGTACTGGGTGATACAC |
| | ATCTGCTCCCAGTGTTCCATCCTCCAGGCTTCGGGCGCTTCTTGCAGAGGCCCAGGTC |
| | ACTCCATGTGGCCACAAAGAGAACCAGCATCCAGCAGCCATGGTTCGCCATAATGACT |
| | GCTCTGCCTCGGTCGTGAGGAGAGGAGAAGCTCGCGGCGCCGCGGCTGTCAGCGACTG |
| | GCTCGGAGGACAGGC |

| | | |
|---|---|---|
| ORF Start: ATG at 201 | | ORF Stop: TGA at 480 |
| SEQ ID NO:16 | 93 aa | MW at 10769.1 kD |

| | |
|---|---|
| NOV7a, | MVKTGRPSSSHYQEDEERDQEDHRWRGEEDHARSSLVIGLRFPLVLGDTHLLPVFHPP |
| CG101362-01 | |
| Protein Sequence | GFGRFLQRPRSLHVATKRTSIQQPRFAIMTALPRS |

Further analysis of the NOV7a protein yielded the following properties shown in Table 7B.

TABLE 7B
Protein Sequence Properties NOV7a

| | |
|---|---|
| PSort | 0.6400 probability located in microbody (peroxisome); |
| analysis: | 0.4500 probability located in cytoplasm; |
| | 0.2288 probability located in lysosome (lumen); |
| | 0.1000 probability located in mitochondrial matrix space |
| SignalP | No Known Signal Sequence Predicted |
| analysis: | |

A search of the NOV7a protein against the Geneseq database, a proprietary database that contains sequences published in patents and patent publication, yielded several homologous proteins shown in Table 7C.

TABLE 7C
Geneseq Results for NOV7a

| | | | Identities/ | |
|---|---|---|---|---|
| | | NOV7a | Similarities | |
| | | Residues/ | for the | |
| Geneseq | Protein/Organism/Length [Patent #, | Match | Matched | Expect |
| Identifier | Date] | Residues | Region | Value |
| AAY19678 | SEQ ID NO 396 from WO9922243 - | 1 . . . 30 | 14/30 (46%) | 0.94 |
| | Homo sapiens, 133 aa. [WO9922243- | 63 . . . 91 | 19/30 (62%) | |
| | A1, 06-MAY-1999] | | | |
| AAB92467 | Human protein sequence SEQ ID | 2 . . . 90 | 24/90 (26%) | 1.2 |
| | NO: 10527 - Homo sapiens, 563 aa. | 318 . . . 398 | 41/90 (44%) | |
| | [EP1074617-A2, 07-FEB-2001] | | | |
| AAU16292 | Human novel secreted protein, Seq ID | 2 . . . 90 | 24/90 (26%) | 1.2 |
| | 1245 - Homo sapiens, 564 aa. | 319 . . . 399 | 41/90 (44%) | |
| | [WO200155322-A2, 02-AUG-2001] | | | |
| ABB50224 | Human transcription factor TRFX-75 - | 2 . . . 90 | 24/90 (26%) | 1.2 |
| | Homo sapiens, 596 aa. | 351 . . . 431 | 41/90 (44%) | |
| | [WO200172777-A2, 04-OCT-2001] | | | |
| AAM33060 | Peptide #7097 encoded by probe for | 5 . . . 30 | 13/26 (50%) | 1.6 |
| | measuring placental gene expression - | 1 . . . 25 | 18/26 (69%) | |
| | Homo sapiens, 49 aa. | | | |
| | [WO200157272-A2, 09-AUG-2001] | | | |

In a BLAST search of public sequence databases, the NOV7a protein was found to have homology to the proteins shown in the BLASTP data in Table 7D.

TABLE 7D
Public BLASTP Results for NOV7a

| | | | Identities/ | |
|---|---|---|---|---|
| | | NOV7a | Similarities | |
| Protein | | Residues/ | for the | |
| Accession | | Match | Matched | Expect |
| Number | Protein/Organism/Length | Residues | Portion | Value |
| Q9D3A0 | 6330414C15RIK PROTEIN - Mus | 1 . . . 30 | 14/30 (46%) | 2.2 |
| | musculus (Mouse), 150 aa. | 51 . . . 79 | 19/30 (62%) | |
| Q9Y269 | Protein HSPC020 - Homo sapiens | 1 . . . 30 | 14/30 (46%) | 2.2 |
| | (Human), and, 121 aa. | 51 . . . 79 | 19/30 (62%) | |
| Q9UKD0 | DNA BINDING PROTEIN P96PIF | 2 . . . 90 | 24/90 (26%) | 2.9 |
| | (GLUCOCORTICOID | 318 . . . 398 | 41/90 (44%) | |
| | MODULATORY ELEMENT | | | |
| | BINDING PROTEIN 1) - Homo | | | |
| | sapiens (Human), 563 aa. | | | |
| Q9NWH1 | HYPOTHETICAL 61.4 KDA | 2 . . . 90 | 24/90 (26%) | 2.9 |
| | PROTEIN - Homo sapiens (Human), | 318 . . . 398 | 41/90 (44%) | |
| | 563 aa. | | | |
| Q9Y692 | GLUCOCORTICOID | 2 . . . 90 | 24/90 (26%) | 3.8 |
| | MODULATORY ELEMENT | 328 . . . 408 | 40/90 (43%) | |
| | BINDING PROTEIN-1 - Homo | | | |
| | sapiens (Human), 573 aa. | | | |

### Example 8

The NOV8 clone was analyzed, and the nucleotide and encoded polypeptide sequences are shown in Table 8A.

TABLE 8A
NOV8 Sequence Analysis

| | |
|---|---|
| SEQ ID NO:17 | 3653 bp |

| | |
|---|---|
| NOV8a, | CGGGATGCCCGGCTTGCTGAATTGGATCACGGGGGCAGCCCTGCCCCTCACCGCGTCT |
| CG101458-01 | |
| DNA Sequence | GATGTTACCTCCTGTGTCAGCGGTTATGCCCTGGGCCTAACTGCCTCCCTCACCTATG |
| | GCAACCTGGATGCCCAGCCCTTCCAGGGTCTCTTCGTGTACCCCCTGGATGAGTGCAC |
| | CACGGTGATCGGCTTTGAGGCAGTCATTGCCGACCGTGTCGTGACAGTACAGATCAAG |
| | GACAAAGCCAAGCTGGAGAGCGGCCACTTCGATGCCTCCCATGTTCGATCCCCAACAG |
| | TCACAGGTAAGGAGACCAGAAGGGCTGCCGCGGGACCTGGGAAGGTGACCTTGGACGA |
| | GGATTTGGAGCGGATCCTGTTCGTGGCCAACCTGGGGACCATTGCCCCCATGGAGAAT |
| | GTCACCATCTTCATCAGCACCTCCTCGGAGCTCCCAACGCTGCCCAGCGGGGCTGTGA |
| | GGGTCCTTCTGCCTGCTGTCTGTGCCCCAACCGTGCCCCAGTTCTGCACCAAGAGCAC |
| | TGGCACCTCCAACCAACAGGCCCAGGGGAAAGACAGGCACTGCTTCGGTGCCTGGGCC |
| | CCGGGCTCCTGGAATAAGTTGTGCCTGGCGACTCTCCTGAACACCGAAGTGTCCAACC |
| | CCATGGAGTATGAGTTCAACTTCCAGCTGGAGATCCGTGGGCCATGTCTGCTCGCAGG |
| | TGTGGAGAGTCCCACTCATGAGATTCGTGCCGACGCCGCCCCATCTGCCCGCTCGGCC |
| | AAGAGCATCATCATCACCTTGGCCAACAAGCACACCTTTGACCGGCCTGTGGAGATCC |
| | TCATCCACCCCAGCGAGCCCCATATGCCCCATGTCCTGATAGAGAAAGGGGACATGAC |
| | CCTGGGAGAGTTTGACCAGCACTTGAAGGGAAGAACAGATTTCATTAAAGGGATGAAG |
| | AAGAAGAGCAGAGCAGAGCGGAAGACAGAAATCATTCGAAAACGCCTCCACAAAGACA |
| | TTCCCCACCACTCCGTCATCATGCTCAACTTCTGTCCCGACCTCCAGTCAGTCCAGCC |
| | GTGCCTGAGAAAGGCCCACGGGGAGTTCATCTTCCTCATTGACAGGAGCAGCAGCATG |
| | AGCGGGATCAGCATGCACCGAGTCAAGGATGCCATGTTGGTGGCCCTTAAGAGCCTCA |
| | TGCCAGCCTGCCTCTTCAATATCATTGGGTTTGGATCCACATTTAAGAGCCTTTTTCC |
| | TTCCAGCCAGACCTACAGTGAGGACAGCTTGGCCATGGCTTGTGATGACATCCAGAGA |
| | ATGAAGGCCQACATGGGTGGGACCAACATCCTTTCCCCTCTCAAGTGGGTCATCAGGC |
| | AGCCAGTGCACCGAGGCCACCCGCGGCTCCTCTTCGTGATCACAGATGGCGCTGTCAA |
| | CAACACAGGGAAAGGTGCTGGAGCTGGTGCGAATCACGCCTTCTCCACCAGGTGCTAT |
| | AGCTTTGGAATTGGACCCAACGTCTGCCACAGACTGGTGAAAGGACTGGCATCTGTGT |
| | CCGAGGGCAGTGCTGAGCTCCTGATGGAGGGGGAGCGGCTGCAACCCAAGATGGTCAA |
| | ATCCTTGAAGAAGGCCATGGCCCCAGTCCTGAGCGATGTGACTGTGGAGTGGATCTTC |
| | CCTGAGACCACTGAGGTCCTGGTCTCACCCGTCAGCGCCAGCTCCCTCTTCCCTGGAG |
| | AACGGCTGGTGGGGTATGGCATTGTATGTGATGCTTCTTTGCACATCTCCAATCCCAG |
| | ATCTGACAAGAGGCGCCGGTACAGCATGCTGCACTCTCAGGAGTCTGGCAGCTCTGTC |
| | TTCTACCACTCTCAGGATGACGGACCCGGGCTGGAAGGTGGAGACTGTGCCAAGAACT |
| | CGGGGGCACCCTTCATCCTAGGGCAGGCCAAAAATGCCCGGCTAGCCAGCGGAGACTC |
| | TACCACCAAGCACGGTCTGAACCTCTCTCAGCGACGGAGGGCATACAGCACCAACCAG |
| | ATCACCAATCACAAGCCCCTCCCAAGAGCCACCATGGCAAGTGACCCCATGCCAGCTG |
| | CCAAGAGATACCCACTGCGGAAAGCCAGGCTGCAGGACCTCACCAACCAGACCAGCCT |
| | GGATGTCCAGCGGTGGCAGATTGATTTGCAGGTATTGCTGAACAGTGGTCAGGACCTG |
| | AACCAGGGCCCCAAACTCCGTGGCCCAGGGGCCCGAAGGCCCTCTCTGCTGCCCCAAG |
| | GCTGCCAGCCCTTCCTGCCCTGGGGCCAGGAGACCCAGGCCTGGAGCCCTGTGAGAGA |
| | GCGGACTTCTGACAGCCGAAGCCCTGGAGATCTGCCCGCAGAGCCGTCCCACCATCCC |
| | TCTGCCTTCGAGACAGAGACGTCCTCGGACTGGGACCCCCCAGCCGAGTCCCAGGAGC |
| | GAGCCAGTCCCAGCAGGCCCGCCACCCCGGCCCCGGTGCTGGGCAAGGCCCTGGTCAA |
| | AGGCCTGCACGACAGCCAACGCCTGCAGTGGGAGGTGAGCTTCGAGCTGGGGACCCCT |
| | GGACCGGAGCGGGGCGGCGCGCAGGATGCCGACCTATGGACCGAGACCTTCCACCACC |
| | TGGCGGCCCGCGCCATCATCCGCGACTTCGAGCAGCTGGCGGAGCGCGAGGGCGAGAT |
| | CGAGCAGGGTTCCAACCGCCGCTACCAAGTGAGCGCCTTGCACACCAGCAAGGCCTGC |
| | AACATCATTAGCAAATACACAGCCTTCGTGCCTGTGGACGTGAGCAAGAGCCGGTACC |
| | TGCCCACCGTGGTGGAGTACCCCAACTCTGGTCGTATGCTTGGCTCTCGGGCCCTGGC |
| | CCAACAGTGGAGGCGCACCTCTTCTGGCTTTGGAAGGCCGCAGACGATGCTTGGAGAA |
| | GATTCGGCACCAGGAAATGGTAAATTTCAGGTCCTAGACATGGAGGCAAGTCCCACTG |
| | CTCTCTTCAGCGAGGCCAGGTCCCCCGGCCGCGAGAAGCACGGTGCTTCTGAAGGTCC |
| | CCAGCGCAGCCTGGCTACAAATACTCTTTCTTCCATGAAGGCCTCAGAGAATCTCTTT |
| | GGATCCAGGCTAAATCTCAACAAGTCCAGGCTACTGACGCGAGCAGCCAAGGGCTTCC |
| | TGAGCAAGCCACTGATCAAAGCTGTGGAGTCGACCTCCGGGAACCAGAGCTTCGACTA |
| | CATACCTCTGGTGTCTCTGCAGCTGGCCTCCGGAGCCTTCCTGCTCAACGAAGCCTTC |
| | TGTGAGGCCACGCACATCCCCATGGAGAAGCTCAAGTGGACGTCCCCCTTCACCTGCC |
| | ATCGAGTGTCCCTCACCACCCGCCCGTCTGAGTCCAAGACCCCGAGTCCCCAGCTGTG |
| | CACCAGCTCCCCGCCTAGGCACCCGTCCTGTGACAGCTTCTCCCTGGAGCCTCTGGCC |
| | AAGGGCAAGCTGGGCCTGGAGCCGAGGGCAGTGGTGGAGCACACTGGGAAGCTGTGGG |
| | CCACGGTGGTGGGGCTGGCATGGCTGGAGCACAGTTCGGCCTCCTACTTCACTGAGTG |
| | GGAGTTGGTGGCTGCCAAGGCCAACTCATGGCTGGAGCAGCAGGAAGTACCCGAGGGC |
| | CGCACGCAGGGCACACTCAAGGCCGCTGCCCGCCAGCTGTTTGTGCTTCTGCGGCACT |
| | GGGATGAGAATCTCGAGTTCAATATGCTCTGCTATAACCCGAATTATGTGTAGTTGA |

| | | |
|---|---|---|
| ORF Start: ATG at 5 | | ORF Stop: TAG at 3647 |
| SEQ ID NO:18 | 1213 aa | MW at 133118.0 kd |

| | |
|---|---|
| NOV8a, | MPGLLNWITGAALPLTASDVTSCVSGYALGLTASLTYGNLEAQPFQGLFVYPLDECTT |
| CG101458-01 | |
| Protein Sequence | VIGFEAVIADRVVTVQIKDKAKLESGHFDASHVRSPTVTGKETRRAAAGPGKVTLDED |
| | LERILFVANLGTIAPMENVTIFISTSSELPTLPSGAVRVLLPAVCAPTVPQFCTKSTG |
| | TSNQQAQGKDRHCFGAWAPGSWNKLCLATLLNTEVSNPMEYEFNFQLEIRGPCLLAGV |
| | ESPTHEIRADAAPSARSAKSIIITLANKHTFDRPVEILIHPSEPHMPHVLIEKGDMTL |
| | GEFDQHLKGRTDFIKGMKKKSRAERKTEIIRKRLHKDIPHHSVIMLNFCPDLQSVQPC |
| | LRKAHGEFIFLIDRSSSMSGISMHRVKDAMLVALKSLMPACLFNIIGFGSTFKSLFPS |
| | SQTYSEDSLAMACDDIQRMKADMGGTNILSPLKNVIRQPVHRGHPRLLFVITDGAVNN |
| | TGKVLELVRNHAFSTRCYSFGIGPNVCHRLVKGLASVSEGSAELLMEGERLQPKMVKS |
| | LKKAIAPVLSDVTVEWIFPETTEVLVSPVSASSLFPGERLVGYGIVCDASLHISNPRS |
| | DKRRRYSMLHSQESGSSVFYHSQDDGPGLEGGDCAKNSCAPFILGQAKNARLASGDST |
| | TKHGLNLSQRRRAYSTNQITNHKPLRRATMASDPMPAAKRYPLRKARLQDLTNQTSLD |
| | VQRWQIDLQVLLNSGQDLNQGPKLRGPGARRPSLLPQGCQPFLPWGQETQAWSPVRER |
| | TSDSRSPGDLPAEPSHHPSAFETETSSDWDPPAESQERASPSRPATPAPVLGKALVKG |
| | LHDSQRLQWEVSFELGTPGPERGGAQDADLWSETFHHLAARAIIRDFEQLAEREGEIE |
| | QGSNRRYQVSALHTSKACNIISKYTAFVPVDVSKSRYLPTVVEYPNSGRMLGSRALAQ |
| | QWRGTSSGFGRPQTMLGEDSAPGNGKFQVLDMEASPTALFSEARSPGREKHGASEGPQ |
| | RSLATNTLSSMKASENLFGSRLNLNKSRLLTRAAKGFLSKPLIKAVESTSGNQSFDYI |
| | PLVSLQLASGAFLLNEAFCEATHIPMEKLKWTSPFTCHRVSLTTRPSESKTPSPQLCT |
| | SSPPMPSCDSFSLEPLAKGKLGLEPPAVVEHTGKLWATVVNGLAWLEHSSASYFTEWE |
| | LVAAKANSWLEQQEVPEGRTQGTLKAAARQLFVLLRHWDENLEFNMLCYNPNYV |

Further analysis of the NOV8a protein yielded the following properties shown in Table 8B.

TABLE 8B
Protein Sequence Properties NOV8a

| | |
|---|---|
| PSort | 0.8700 probability located in nucleus; 0.8500 probability |
| analysis: | located in endoplasmic reticulum (membrane); |
| | 0.7900 probability located in plasma membrane; |
| | 0.3325 probability located in microbody (peroxisome) |
| SignalP | Cleavage site between residues 19 and 20 |
| analysis: | |

A search of the NOV8a protein against the Geneseq database, a proprietary database that contains sequences published in patents and patent publication, yielded several homologous proteins shown in Table 8C.

TABLE 8C
Geneseq Results for NOV8a

| | | NOV8a | Identities/ | |
|---|---|---|---|---|
| | | Residues/ | Similarities for | |
| Geneseq | Protein/Organism/Length [Patent | Match | the Matched | Expect |
| Identifier | #, Date] | Residues | Region | Value |
| AAB82047 | Human mast cell surface antigen - | 13 . . . 565 | 168/565 (29%) | 2e−59 |
| | Homo sapiens, 786 aa. | 15 . . . 500 | 269/565 (46%) | |
| | [JP2001025388-A, 30-JAN-2001] | | | |
| AAY82530 | Human neurotransmitter associated | 1034 . . . 1211 | 82/194 (42%) | 1e−32 |
| | protein sequence SEQ ID NO: 6 - | 16 . . . 207 | 105/194 (53%) | |
| | Homo sapiens, 210 aa. | | | |
| | [WO200012685-A2, 09-MAR-2000] | | | |
| AAU33242 | Novel human secreted protein | 36 . . . 568 | 120/537 (22%) | 4e−23 |
| | #3733 - Homo sapiens, 1730 aa. | 650 . . . 1096 | 214/537 (39%) | |
| | [WO200179449-A2, 25-OCT-2001] | | | |
| AAB51022 | Human minor vault protein p193 - | 36 . . . 568 | 120/537 (22%) | 6e−23 |
| | Homo sapiens, 1724 aa. | 644 . . . 1090 | 214/537 (39%) | |
| | [US6156879-A, 05-DEC-2000] | | | |
| AAY54373 | cDNA sequence encoding the | 36 . . . 568 | 120/537 (22%) | 6e−23 |
| | human minor vault protein p193 - | 644 . . . 1090 | 214/537 (39%) | |
| | Homo sapiens, 1724 aa. | | | |
| | [WO9962547-A1, 09-DEC-1999] | | | |

In a BLAST search of public sequence databases, the NOV8a protein was found to have homology to the proteins shown in the BLASTP data in Table 8D.

TABLE 8D
Public BLASTP Results for NOV8a

| | | NOV8a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9CUE8 | 4931403E03RIK PROTEIN - Mus | 1 . . . 1208 | 883/1218 (72%) | 0.0 |
| | musculus (Mouse), 1209 aa | 1 . . . 1209 | 1012/1218 (82%) | |
| | (fragment). | | | |
| Q96M71 | CDNA FLJ32784 FIS, CLONE | 588 . . . 953 | 362/369 (98%) | 0.0 |
| | TESTI2002245 - Homo sapiens | 1 . . . 367 | 362/369 (98%) | |
| | (Human), 424 aa. | | | |
| Q9BVH8 | HYPOTHETICAL 106.2 KDA | 274 . . . 1211 | 311/1047 (29%) | e−106 |
| | PROTEIN - Homo sapiens | 32 . . . 998 | 467/1047 (43%) | |
| | (Human), 1001 aa (fragment). | | | |
| O75668 | DJ745E8.1 (BREAST CANCER | 417 . . . 564 | 148/148 (100%) | 4e−80 |
| | SUPPRESSOR CANDIDATE 1 | 1 . . . 148 | 148/148 (100%) | |
| | (BCSC-1) LIKE) - Homo sapiens | | | |
| | (Human), 148 aa (fragment). | | | |
| Q9CTV9 | 5830475I06RIK PROTEIN - Mus | 13 . . . 565 | 165/567 (29%) | 5e−57 |
| | musculus (Mouse), 565 aa | 15 . . . 500 | 259/567 (45%) | |
| | (fragment). | | | |

PFam analysis predicts that the NOV8a protein contains the domains shown in the Table 8E.

TABLE 8E
Domain Analysis of NOV8a

| | | Identities/ | |
|---|---|---|---|
| | | Similarities | |
| | | for the Matched | Expect |
| Pfam Domain | NOV8a Match Region | Region | Value |
| vwa | 355 . . . 523 | 37/203 (18%) | 0.021 |
| | | 107/203 (53%) | |

### Example 9

The NOV9 clone was analyzed, and the nucleotide and encoded polypeptide sequences are shown in Table 9A.

TABLE 9A
NOV9 Sequence Analysis

| | |
|---|---|
| SEQ ID NO:19 | 868 bp |

| | |
|---|---|
| NOV9a, | CGTTTTCTTCTACAATGTCTGAAGAAGTGACCTACGCGACACTCACATTTCAGGATTC |
| CG101475-01 | |
| DNA Sequence | TGCTGGAGCAAGGAATAACCGAGTATGGAAATAACCTAAGAAAAGAGGTCATCCAGCT |
| | CCATCTCCCATTTGGCGTCATGCTGCTCTGGGTCTGGTAACTCTTTGCCTGATGTTGC |
| | TGATTGGGCTGGTGACATTGGGGATGATGTGTTTGCAGATATCTAATGACATTAACTC |
| | AGATTCAGAGAAATTGAGTCAACTTCAGAAAACCATCCAACAGCAGCAGGATAACTTA |
| | TCCCAGCAACTGGGCAACTCCAACAACTTGTCCATGGAGGAGGAATTTCTCAAGTCAC |
| | AGATCTCCAGTGTACTGAAGAGGCAGGAACAAATGGCCATCAAACTGTGCCAAGAGCT |
| | AATCATTCATTTTTCAGACCACAGATGTAATCCATGTCCTAAGATGTGGCAATGGTAC |
| | CAAAATAGTTGCTACTATTTTACAACAAATGAGGAGAAAACCTGGGCTAACAGTAGAA |
| | AGGACTGCATAGACAAAGAACTCCACCCTAGTGAAGATAGACAGTTTGGAAGAAAGGA |
| | TTTTCTTATGTCACAGCCATTACTCATGTTTTCGTTCTTTTGGCTGGGATTATCATGG |
| | GACTCCTCTGGCAGAAGTTGGTTCTGGGAAGATGGCTCTGTTCCCTCTCCATCCTTGA |
| | GTACTAAAGAACTTGACCAGATCAATGGATCCAAAGGATGTGCTTATTTTCAAAAAGG |
| | AAATATTTATATTTCTCGCTGTAGTGCTGAAATTTTTTGGATTTGCGAGAAGACAGCT |
| | GCCCCAGTGAGACTGAGGATTTGGATTAGTATGCTTCTTCCAAATTCTCCAAGAA |

| | | |
|---|---|---|
| ORF Start: ATG at 15 | | ORF Stop: TAG at 840 |
| SEQ ID NO:20 | 275 aa | MW at 31470.4 kD |

| | |
|---|---|
| NOV9a, | MSEEVTYATLTFQDSAGARNNRDGNNLRKRGHPAPSPIWRHAALGLVTLCLMLLIGLV |
| CG101475-01 | |
| Protein Sequence | TLGMMCLQISNDINSDSEKLSQLQKTIQQQQDNLSQQLGNSNNLSMEEEFLKSQISSV |
| | LKRQEQMAIKLCQELIIHFSDHRCNPCPKMWQWYQNSCYYFTTNEEKTWANSRKDCID |
| | KNSTLVKIDSLEEKDFLMSQPLLMFSFFWLGLSWDSSGRSWFWEDGSVPSPSLSTKEL |
| | DQINGSKGCAYFQKGNIYISRCSAEIFWICEKTAAPVKTEDLD |

| | |
|---|---|
| SEQ ID NO:21 | 819 bp |

| | |
|---|---|
| NOV9b, | ACACTCACATTTCAGGATTCTGCTGGAGCAAGGAATAACCGAGATGGAAATAACCTAA |
| CG101475-02 | |
| DNA Sequence | GAAAAAGAGGGCATCCAGCTCCATCTCCCATTTGGCGTCATGCTGCTCTGGGTCTGGT |
| | AACTCTTTGCCTGATGTTGCTGATTGGGCTGGTGACGTTGGGGATGATGTTTTTGCAG |
| | ATATCTAATGACATTAACTCAGATTCAGAGAAATTGAGTCAACTTCAGAAAACCATCC |
| | AACAGCAGCAGGATAACTTATCCCAGCAACTGGGCAACTCCAACAACTTGTCCATGGA |
| | GGAGGAATTTCTCAGTCACAGATCTCCAGTGTACTGAAGAAGGCAGGAACAAATGGCC |
| | ATCAAACTGTGCCAAGAGCTAATCATTCATACTTCAGACCACAGATGTAATCCATGTC |
| | CTAAGATGTGGCAATGGTACCAAAATAGTTGCTACTATTTTACAACAAATGAGGAGAA |
| | AACCTGGGCTAACAGTAGAAAGGACTGCATAGACAAGAACTCCACCCTAGTGAAGATA |
| | GACAGTTTGGAAGAAAAGGATTTTCTTATGTCACAGCCATTACTCATGTTTTCGTTCT |
| | TTTGGCTGGGATTATCATGGGACTCCTCTGGCAGAAGTTGCTTCTGGGAAGATGGCTC |
| | TGTTCCCTCTCCATCCTTATTTAGTACTAAAGAACTTGACCAGATCAATGGATCCAAA |
| | GGATGTGCTTATTTTCAAAGGAAAAATATTTATATTTCTCGCTGTAGTGCTGAAATTT |
| | TTTGGATTTGCGAGAAAGACAGCTGCCCCAGTGAAGACTGAGGATTTGGATTAGAGGG |
| | CGATTCC |

| | | |
|---|---|---|
| ORF Start: at 1 | | ORF Stop: TAG at 805 |
| SEQ ID NO: 22 | 268 aa | MW at 30704.5 kD |

| | |
|---|---|
| NOV9b, | TLTFQDSAGARNNRDGNNLRKRGHPAPSPIWRHAALGLVTLCLMLLIGLVTLGMMFLQ |
| CG101475-02 | |
| Protein Sequence | ISNDINSDSEKLSQLQKTIQQQQDNLSQQLGNSNNLSMEEEFLKSQISSVLKRQEQMA |
| | IKLCQELIIHTSDHRCNPCPKMWQWYQNSCYYFTTNEEKTWANSRKDCIDKNSTLVKI |
| | DSLEEKDFLMSQPLLMFSFFWLGLSWDSSGRSWFWEDGSVPSPSLFSTKELDQINGSK |
| | GCAYFQKGNIYISRCSAEIFWICEKTAAPVKTEDLD |

Sequence comparison of the above protein sequences yields the following sequence relationships shown in Table 9B.

TABLE 9B
Comparison of NOV9a against NOV9b.

| | | Identities/ |
|---|---|---|
| | NOV9a Residues/ | Similarities |
| Protein Sequence | Match Residues | for the Matched Region |
| NOV9b | 9 . . . 275 | 241/268 (89%) |
| | 1 . . . 268 | 241/268 (89%) |

Further analysis of the NOV9a protein yielded the following properties shown in Table 9C.

TABLE 9C
Protein Sequence Properties NOV9a

| | |
|---|---|
| PSort | 0.7900 probability located in plasma membrane; 0.3000 probability located in |
| analysis: | Golgi body; 0.2000 probability located in endoplasmic reticulum (membrane); |
| | 0.1000 probability located in mitochondrial inner membrane |
| SignalP | Cleavage site between residues 62 and 63 |
| analysis: | |

A search of the NOV9a protein against the Geneseq database, a proprietary database that contains sequences published in patents and patent publication, yielded several homologous proteins shown in Table 9D.

TABLE 9D
Geneseq Results for NOV9a

| | | NOV9a | Identities/ | |
|---|---|---|---|---|
| | Protein/Organism/ | Residues/ | Similarities for | |
| Geneseq | Length [Patent | Match | the Matched | Expect |
| Identifier | #, Date] | Residues | Region | Value |
| AAU29320 | Human PRO polypeptide sequence | 1 . . . 227 | 224/227 (98%) | e−131 |
| | #297 - Homo sapiens, 232 aa. | 1 . . . 227 | 225/227 (98%) | |
| | [WO200168848-A2, 20 SEP. 2001] | | | |
| AAM79324 | Human protein SEQ ID NO 2970 - | 1 . . . 270 | 91/270 (33%) | 3e−37 |
| | Homo sapiens, 289 aa. | 25 . . . 280 | 147/270 (53%) | |
| | [WO200157190-A2, 09 AUG. | | | |
| | 2001] | | | |
| ABB11776 | Human macrophage Ag homologue, | 1 . . . 270 | 91/270 (33%) | 3e−37 |
| | SEQ ID NO: 2146 - Homo sapiens, | 25 . . . 280 | 147/270 (53%) | |
| | 289 aa. [WO200157188-A2, 09 | | | |
| | AUG. 2001] | | | |
| AAM78340 | Human protein SEQ ID NO 1002 - | 1 . . . 270 | 88/270 (32%) | 1e−35 |
| | Homo sapiens, 265 aa. | 1 . . . 256 | 147/270 (53%) | |
| | [WO200157190-A2, 09 AUG. | | | |
| | 2001] | | | |
| AAY02283 | Secreted protein clone br342_11 | 1 . . . 270 | 88/270 (32%) | 1e−35 |
| | polypeptide sequence - Homo | 1 . . . 256 | 147/270 (53%) | |
| | sapiens, 265 aa. [WO9918127-A1, | | | |
| | 15 APR. 1999] | | | |

In a BLAST search of public sequence databases, the NOV9a protein was found to have homology to the proteins shown in the BLASTP data in Table 9E.

TABLE 9E
Public BLASTP Results for NOV9a

| | | NOV9a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9D403 | 4933425B16RIK PROTEIN - Mus | 1 . . . 275 | 197/276 (71%) | e−113 |
| | musculus (Mouse), 275 aa. | 1 . . . 275 | 227/276 (81%) | |
| AAL95693 | C-TYPE LECTIN PROTEIN | 1 . . . 270 | 88/270 (32%) | 2e−35 |
| | CLL-1 - Homo sapiens (Human), | 1 . . . 256 | 147/270 (53%) | |
| | 265 aa. | | | |
| Q9NZH3 | C-TYPE LECTIN-LIKE | 28 . . . 274 | 83/249 (33%) | 5e−33 |
| | RECEPTOR-1 - Homo sapiens | 36 . . . 269 | 131/249 (52%) | |
| | (Human), 280 aa. | | | |
| Q9XTA8 | LECTIN-LIKE OXIDIZED LDL | 36 . . . 272 | 79/247 (31%) | 5e−27 |
| | RECEPTOR - Oryctolagus | 36 . . . 278 | 124/247 (49%) | |
| | cuniculus (Rabbit), 278 aa. | | | |
| P78380 | LECTIN-LIKE OXIDIZED LDL | 36 . . . 266 | 79/245 (32%) | 3e−24 |
| | RECEPTOR - Homo sapiens | 32 . . . 268 | 124/245 (50%) | |
| | (Human), 273 aa. | | | |

PFam analysis predicts that the NOV9a protein contains the domains shown in the Table 9F.

TABLE 9F
Domain Analysis of NOV9a

| | | Identities/ | |
|---|---|---|---|
| | | Similarities for | Expect |
| Pfam Domain | NOV9a Match Region | the Matched Region | Value |
| lectin_c | 161 . . . 264 | 29/125 (23%) | 7.4e−06 |
| | | 61/125 (49%) | |

### Example 10

The NOV 10 clone was analyzed, and the nucleotide and encoded polypeptide sequences are shown in Table 10A.

TABLE 10A
NOV10 Sequence Analysis

| | SEQ ID NO: 23 | 516 bp | |
|---|---|---|---|
| NOV10a, | CACTGCGCATGCTATTTGGGCGCCCACCTCAGTGCACATGTTCACTGGGCGTCTTCTA | | |
| CG101772-01 | CTCTACCCCTTCGCCCTCGTGGGGGTGTGAGGGTCGCGTTCCTGCTGTCTGGACTTTT | | |
| DNA Sequence | TCTGTCCCACTGAGACGCAATGTATCGATAACAAAACTTTTTATCTGCACACACACAC | | |
| | ACCACCAACTGAAAGTCGGGATCCTGCACCTGGTCAGGAGAGAGAAGAAGATCAGGGT | | |
| | GCAGCTGAGACTCAATGCCTGACCTGGAAGCTGATCTCCAGGAGCTGTCTCAGTCAAA | | |
| | GACTGGGGGTGAATGTGGAAATGAAGATTCTGCCAAAATCAGAACAATTTAAAATGCC | | |
| | AGAAGGAGGTATGCTATCCATTATTATGTGCTTTCTGTTTTCCACAATATTATACTTT | | |
| | TGATAATAAAAGAGAACATTACTATCCCTTTAAAATCAGAGTTCAAATGCAG | | |
| | ORF Start: ATG at 9 | ORF Stop: TGA at 465 | |
| | SEQ ID NO: 24 | 152 aa | MW at 17265.6kD |

| | |
|---|---|
| NOV10a, | MLFGRPPQCTCSLGVFYSTPSPSWGCEGRVPAVWTFSVPLRRNVSITKLFICTHTHTH |
| CG101772-01 | THPWFQEPGDEEPQQEEPPTESRDPAPGQEREEDQGAAETQCLTWKLISRSCLSQRLG |
| Protein Sequence | VNVEMKILPKSEQFKMPEGGMLSIIMCFLFSTILYF |

Further analysis of the NOV10a protein yielded the following properties shown in Table 10B.

TABLE 10B
Protein Sequence Properties NOV10a

| | |
|---|---|
| PSort | 0.9190 probability located in plasma membrane; 0.2000 probability located in |
| analysis: | lysosome (membrane); 0.1021 probability located in microbody (peroxisome); |
| | 0.1000 probability located in endoplasmic reticulum (membrane) |
| SignalP | No Known Signal Sequence Predicted |
| analysis: | |

A search of the NOV10a protein against the Geneseq database, a proprietary database that contains sequences published in patents and patent publication, yielded several homologous proteins shown in Table 10C.

TABLE 10C
Geneseq Results for NOV10a

| | | | Identities/ | |
|---|---|---|---|---|
| | | NOV10a | Similarities | |
| | Protein/Organism/ | Residues/ | for the | |
| Geneseq | Length [Patent | Match | Matched | Expect |
| Identifier | #, Date] | Residues | Region | Value |
| AAM39588 | Human polypeptide SEQ ID NO | 65 . . . 136 | 51/78 (65%) | 4e−20 |
| | 2733 - Homo sapiens, 111 aa. | 28 . . . 105 | 56/78 (71%) | |
| | [WO200153312-A1, 26 JUL. 2001] | | | |
| AAM41374 | Human polypeptide SEQ ID NO | 65 . . . 135 | 52/77 (67%) | 2e−19 |
| | 6305 - Homo sapiens, 106 aa. | 29 . . . 105 | 57/77 (73%) | |
| | [WO200153312-A1, 26 JUL. 2001] | | | |
| ABG05297 | Novel human diagnostic protein | 65 . . . 136 | 48/78 (61%) | 8e−19 |
| | #5288 - Homo sapiens, 112 aa. | 29 . . . 106 | 56/78 (71%) | |
| | [WO200175067-A2, 11 OCT. 2001] | | | |
| ABG05297 | Novel human diagnostic protein | 65 . . . 136 | 48/78 (61%) | 8e−19 |
| | #5288 - Homo sapiens, 112 aa. | 29 . . . 106 | 56/78 (71%) | |
| | [WO200175067-A2, 11 OCT. 2001] | | | |
| ABG27048 | Novel human diagnostic protein | 64 . . . 135 | 45/78 (57%) | 5e−15 |
| | #27039 - Homo sapiens, 249 aa. | 70 . . . 147 | 53/78 (67%) | |
| | [WO200175067-A2, 11 OCT. 2001] | | | |

In a BLAST search of public sequence databases, the NOV10a protein was found to have homology to the proteins shown in the BLASTP data in Table 10D.

TABLE 10D
Public BLASTP Results for NOV10a

| | | NOV10a | Identities/ | |
|---|---|---|---|---|
| Protein | | Residues/ | Similarities for | |
| Accession | | Match | the Matched | |
| Number | Protein/Organism/Length | Residues | Portion | Expect Value |
| Q8WTP9 | XAGE-3 PROTEIN - Homo | 65 . . . 136 | 51/78 (65%) | 1e−19 |
| | sapiens (Human), 111 aa. | 28 . . . 105 | 56/78 (71%) | |
| Q8WYS9 | HYPOTHETICAL 12.3 KDA | 65 . . . 136 | 51/78 (65%) | 1e−19 |
| | PROTEIN - Homo sapiens | 28 . . . 105 | 56/78 (71%) | |
| | (Human), 111 aa. | | | |
| Q9HD64 | G antigen family D 2 protein | 1 . . . 136 | 59/149 (39%) | 3e−18 |
| | (XAGE-1) - Homo sapiens | 1 . . . 140 | 76/149 (50%) | |
| | (Human), 146 aa. | | | |
| Q8WWM1 | XAGE-5 PROTEIN - Homo | 65 . . . 136 | 45/78 (57%) | 9e−15 |
| | sapiens (Human), 108 aa. | 25 . . . 102 | 53/78 (67%) | |
| Q96GT9 | SIMILAR TO G ANTIGEN 8 | 65 . . . 136 | 39/78 (50%) | 7e−13 |
| | (XAGE-2 PROTEIN) - Homo | 28 . . . 105 | 53/78 (67%) | |
| | sapiens (Human), 111 aa. | | | |

### Example 11

The NOV11 clone was analyzed, and the nucleotide and encoded polypeptide sequences are shown in Table 11A.

TABLE 11BA
NOV11 Sequence Analysis

| | |
|---|---|
| SEQ ID NO:25 | 709 bp |

| | |
|---|---|
| NOV11a, | CGGCCGGTTTTGGTAGGCCCGGGCCGCCGCCAGGCCTCCGCCTGAGCCCGCACCCGCC |
| CG102532-01 | |
| DNA Sequence | ATGGACAACTACGCAGATCTTTCGGATACCGAGCTGACCACCTTGCTGCGCCGGTACA |
| | ACATCCCGCACGGGCCTGTAGTAGGATCAACTCGTAGGCTTTACGAGAAGAAGATCTT |
| | CGAGTACGAGACCCAGAGGCGGCGGCTCTCGCCCCCCAGCTCGTCCGCCGCCTCCTCT |
| | TATAGCTTCTCTGACTTGAATTCGACTAGAGGGGATGCAGATATGTATGATCTTCCCA |
| | AGAAAGAGGACGCTTTACTCTACCAGAGCAAGGGCTACAATGACGATCTTTTGTCTTC |
| | TTCTGAAGAGGAGTGCAAGGATAGGGAACGCCCCATGTACGGCCGGGACAGTGCCTAC |
| | CAGAGCATCACGCACTACCGCCCTGTTTCAGCCTCCAGGAGCTCCCTGGACCTGTCCT |
| | ATTATCCTACTTCCTCCTCCACCTCTTTTATGTCCTCCTCATCATCTTCCTCTTCATG |
| | GCTCACCCGCCGTGCCATCCGGCCTGAAAACCGTGCTCCTGGGGCTGGGCTGGGCCAG |
| | GATCGCCAGGTCCCGCTCTGGGGCCAGCTGCTGCTTTTCCTGGTCTTTGTGATCGTCC |
| | TCTTCTTCATTTACCACTTCATGCAGGCTGAAGAAGGCAACCCCTTCTGACTGCAGCC |
| | AAGCTAATTCCGG |

| | | |
|---|---|---|
| ORF Start: ATG at 59 | | ORF Stop: TGA at 686 |
| SEQ ID NO:26 | 209 aa | MW at 23844.1 kD |

| | |
|---|---|
| NOV11a, | MDNYADLSDTELTTLLRRYNIPHGPVVGSTRRLYEKKIFEYETQRRRLSPPSSSAASS |
| CG102532-01 | |
| Protein Sequence | YSFSDLNSTRGDADMYDLPKKEDALLYQSKGYNDDLLSSSEEECKDRERPMYGRDSAY |
| | QSITHYRPVSASRSSLDLSYYPTSSSTSFMSSSSSSSSWLTRPAIRPENPAPGAGLGQ |
| | DRQVPLWGQLLLFLVFVIVLFFIYHFMQAEEGNPF |

Further analysis of the NOV11a protein yielded the following properties shown in Table 11B.

TABLE 11B
Protein Sequence Properties NOV11a

| | |
|---|---|
| PSort | 0.8500 probability located in endoplasmic reticulum (membrane); 0.6000 probability |
| analysis: | located in nucleus; 0.4400 probability located in plasma |
| | membrane; 0.2323 probability located in microbody (peroxisome) |
| SignalP | No Known Signal Sequence Predicted |
| analysis: | |

A search of the NOV11a protein against the Geneseq database, a proprietary database that contains sequences published in patents and patent publication, yielded several homologous proteins shown in Table 11C.

TABLE 11C
Geneseq Results for NOV11a

| | | NOV11a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAY41294 | Human emerin sequence | 1 . . . 209 | 209/254 (82%) | e−112 |
| | (EMD_HU) - Homo sapiens, 254 | 1 . . . 254 | 209/254 (82%) | |
| | aa. [WO9954468-A1, 28 OCT. | | | |
| | 1999] | | | |
| AAG02346 | Human secreted protein, SEQ ID | 1 . . . 51 | 51/51 (100%) | 2e−23 |
| | NO: 6427 - Homo sapiens, 51 aa. | 1 . . . 51 | 51/51 (100%) | |
| | [EP1033401-A2, 06 SEP. 2000] | | | |
| AAY41297 | Human thymopoietin gamma | 6 . . . 209 | 60/231 (25%) | 7e−10 |
| | sequence - Homo sapiens, 345 aa. | 114 . . . 333 | 107/231 (45%) | |
| | [WO9954468-A1, 28 OCT. 1999] | | | |
| AAR93188 | Thymopoietin-gamma - Homo | 6 . . . 209 | 60/231 (25%) | 7e−10 |
| | sapiens, 345 aa. [WO9609526-A1, | 114 . . . 333 | 107/231 (45%) | |
| | 28 MAR. 1996] | | | |
| AAR76499 | Human thymopoietin-gamma - | 6 . . . 209 | 60/231 (25%) | 7e−10 |
| | Homo sapiens, 345 aa. | 114 . . . 333 | 107/231 (45%) | |
| | [WO9517205-A1, 29 JUN. 1995] | | | |

In a BLAST search of public sequence databases, the NOV11a protein was found to have homology to the proteins shown in the BLASTP data in Table 11D.

TABLE 11D
Public BLASTP Results for NOV11a

| | | NOV11a | Identities/ | |
|---|---|---|---|---|
| Protein | | Residues/ | Similarities for | |
| Accession | | Match | the Matched | Expect |
| Number | Protein/Organism/Length | Residues | Portion | Value |
| P50402 | Emerin - Homo sapiens | 1 . . . 209 | 209/254 (82%) | e−111 |
| | (Human), 254 aa. | 1 . . . 254 | 209/254 (82%) | |
| Q63190 | Emerin - Rattus norvegicus | 1 . . . 209 | 162/256 (63%) | 1e−81 |
| | (Rat), 260 aa. | 1 . . . 256 | 182/256 (70%) | |
| O08579 | Emerin - Mus musculus | 1 . . . 209 | 162/255 (63%) | 1e−81 |
| | (Mouse), 259 aa. | 1 . . . 255 | 182/255 (70%) | |
| Q61032 | THYMOPOIETIN GAMMA - | 6 . . . 209 | 66/231 (28%) | 2e−11 |
| | Mus musculus (Mouse), 342 aa. | 112 . . . 331 | 106/231 (45%) | |
| AAC25390 | THYMOPOIETIN GAMMA - | 6 . . . 209 | 60/231 (25%) | 2e−09 |
| | Homo sapiens (Human), 345 aa. | 114 . . . 333 | 107/231 (45%) | |

PFam analysis predicts that the NOV11a protein contains the domains shown in the Table 11E.

TABLE 11E
Domain Analysis of NOV11a

| | | Identities/ | |
|---|---|---|---|
| Pfam | | Similarities | Expect |
| Domain | NOV11a Match Region | for the Matched Region | Value |
| LEM | 1 . . . 44 | 22/47 (47%) | 4.4e−24 |
| | | 43/47 (91%) | |

### Example 12

The NOV12 clone was analyzed, and the nucleotide and encoded polypeptide sequences are shown in Table 12A.

TABLE 12A
NOV12 Sequence Analysis

| | SEQ ID NO:27 | 2812 bp |
|---|---|---|
| NOV12a, | CATTGAGTCGGCTTTTCTACTGCTTCGGCTAGGGTACCTTGTGACCATGTCTTCCAAG | |
| CG102575-01 | AAGAATAGAAAGCGGTTGAACCAAAGCGCGGAAAATGGTTCGTCCTTGCCCTCTGCTG | |
| DNA Sequence | CTTCCTCTTGTGCGGAGGCACGGGCTCCTTCTGCTGGATCAGACTTCGCGGCAACCTC | |
| | CGGGACTCTGACGGTGACCAACTTATTAGAAAAGGGTAAAATTCCTAAAACATTCCAG | |
| | AATTCCCTTATTCATCTTGGACTCAACACTATGAAGTCTGCAAATATATGTATAGGTC | |
| | GACCAGTGTTGCTTACTAGTTTGAACGGAAAGCAAGAGGTATATACAGCCTGGCCTAT | |
| | GGCAGGATTTCCTGGAGGCAAGGTCGGCCTGAGTGAAATGGCACAGAAAAATGTGGGT | |
| | GTGAGGCCTGGTGATGCCATCCAGGTCCAGCCTCTTGTGGGTGCTGTGCTACAGGCTG | |
| | AGGAAATGGATGTGGCACTGAGTGACAAAGATATGGAAATTAATGAAGAAGAACTGAC | |
| | TGGTTGTATCCTGAGAAAACTAGATGGCAAGATTGTTTTACCAGGCAACTTTCTGTAT | |
| | TGTACATTCTATGGACGACCGTACAAGCTGCAAGTATTGCGAGTGAAAGGGGCAGATG | |
| | GCATGATATTGGGAGGGCCTCAGAGTGACTCTGACACTGATGCCCAAAGAATGGCCTT | |
| | TGAACAGTCCAGCATGGAAACCAGTAGCCTGGAGTTATCCTTACAGCTAAGCCAGTTA | |
| | GATCTGGAGGATACCCAGATCCCAACATCAAGAAGTACTCCTTATAAACCAATTGATG | |
| | ACAGAATTACAAATAAAGCCAGTGATGTTTTGCTGGATGTTACACAGAGCCCTGGAGA | |
| | TGGCAGTGGACTTATGCTAGAGGAAGTCACAGGTCTTAAATGTAATTTTGAATCTGCC | |
| | AGAGAAGGAAATGAGCAACTTACTGAAGAAGAGAGACTGCTAAAGTTCAGCATAGGAG | |
| | CAAAGTGCAATACTGATACTTTTTATTTTATTTCTTCAACAACAAGAGTCAATTTTAC | |
| | AGAGATTGATAAAAATTCAAAAGAGCAAGACAGTGATGTTAAAAGTAACTATGACCAT | |
| | GATAGAGGATTAAGTAGCCAGCTGAAAGCAATTAGAGAAATAATTGAATTGCCCCTCA | |
| | AAATTCCTGCCCCTAGAGGATTGTTACTTTATGGTCCTCCATGTACTGGAAAAACAAT | |
| | GATCGCCAGGGCTGTTGCTAATGAATTTGGAGCCTATGTTTCTGTAATTAATGGTCCT | |
| | GAAATTATAAGCAAGTTCTATGGTGAGACTGAAGCAAAGTTACGTCAGATATTTGCTG | |
| | AAGCCACTCTAAGACACCCATCAATTATTTTTATTGATGAGCTGGATGCACTTTGTCC | |
| | GAAAAGAGAGGGGGCCCAGAATGAAGTGGAAAAAAGAGTTGTGGCTTCACTCTTAACA | |
| | CTGATGGATGGCATTGGTTCAGAAGTAAGTGAAGGACAAGTGTTGGTTCTTGGGGCCA | |
| | CAAATCGCCCTCATGCCTTGGATGCTGCTCTCCGAAGACCTGGGCGATTTGATAAAGA | |
| | GATTGAGATTGGAGTTCCCAATGCTCAGGACCGGCTAGATATTCTCCAGAAACTGCTT | |
| | CGAAGGGTACCCCATTTGCTCACTGAGGCTGAGCTGCTGCAGCTGGCAAATAGTGCTC | |
| | ATGGATACGTTGGAGCAGACTTGAAAGTCTTGTGTAATGAAGCAGGTCTCTGTGCCTT | |
| | GCGGAGAATCCTGAAAAAACAGCCTAACCTCCCTGATGTCAAGGTGGCTGGACTGGTG | |
| | AAGATTACTCTGAAGGATTTCTTGCAGGCAATGAATGATATCAGACCCAGTGCCATGA | |
| | GGGAAATAGCAATTGATGTCCCAAATGTAAGTTATGATGATGTTGGTGGAGTTAGAAA | |
| | GCAAATGGCCCAAATCAGAGAGCTTGTTGAGCTTCCACTACGCCATCCTCAACTTTTC | |
| | AAATCTATTGGTATTCCTGCCCCTAGAGGATTGTTACTTTATGGTCCTCCATGTACTG | |
| | GAAAAACAATGATCGCCAGGGCTGTTGCTAATGAATTTGGAGCCTATGTTTCTGTAAT | |
| | TAATGGTCCTGAAATTATAAGCAAGTATGTTGGTGAGAGTGAACGTGCTGTGCGACAA | |
| | GTTTTTCAACGAGCCAAGAACTCAGCACCATCAATTATTTTTATTGATGAGCTGGATG | |
| | CACTTTGTCCGAAAAGAGAGGGGGCCCAGAATGAAGTGGAAAAAAGAGTTGTGGCTTC | |
| | ACTCTTAACACTGATGGATGGCATTGGTTCAGTAAGTATAGTGTTGGTTCTTGGGGCC | |
| | ACAAATCGCCCTCATGCCTTGGATGCTGCTCTCCGAAGACCTGGGCGATTTGATAAAG | |
| | AGATTGAGATTGGAGTTCCCAATGCTCAGGACCGGCTAGATATTCTCCAGAAACTGCT | |
| | TCGAAGGGTACCCCATTTGCTCACTGAGGCTGAGCTGCTGCAGCTGGCAAATAGTGCT | |
| | CATGGATACGTTGGAGCAGACTTGAAAGTCTTGTGTAATGAAGCAGGTGAGTGTGGTT | |
| | TGCTATGGGACATTCAAGCCAATCTCATCATGAAAAGACATTTCACTCAGGCCTTGAG | |
| | CACTGTGACACCTAGAATTCCTGAGTCATTGAGACGTTTTTATGAAGATTATCAAGAG | |
| | AAGAGTGGGCTGCATACACTCTGAGAAAATATATATATTCAAGATGCTGAAAATCCTT | |
| | TCCAGAGAAAATTGTTTCTTTTTAAAATTTTTGAGAGTGTTAAAAAAAATTTTACTAG | |
| | GCAAAATGTTTGAAGTATGTTCAGTAGA | |
| | ORF Start: ATG at 47 | ORF Stop: TGA at 2690 |
| | SEQ ID NO:28 | 881 aa MW at 96419.5 kD |
| NOV12a, | MSSKKNRKRLNQSAENGSSLPSAASSCAEARAPSAGSDFAATSGTLTVTNLLEKGKIP | |
| CG102575-01 | KTFQNSLIHLGLNTMKSANICIGRPVLLTSLNGKQEVYTAWPMAGFPGGKVGLSEMAQ | |
| Protein Sequence | KNVGVRPGDAIQVQPLVGAVLQAEEMDVALSDKDMEINEEELTGCILRKLDGKIVLPG | |
| | NFLYCTFYGRPYKLQVLRVKGADGMILGGPQSDSDTDAQRMAFEQSSMETSSLELSLQ | |
| | LSQLDLEDTQIPTSRSTPYKPIDDRITNKASDVLLDVTQSPGDGSGLMLEEVTGLKCN | |
| | FESAREGNEQLTEEERLLKFSIGAKCNTDTFYFISSTTRVNFTEIDKNSKEQDSDVKS | |
| | NYDHDRGLSSQLKAIREIIELPLKIPAPRGLLLYGPPCTGKTMIARAVANEFGAYVSV | |
| | INGPEIISKFYGETEAKLRQIFAEATLRHPSIIFIDELDALCPKREGAQNEVEKRVVA | |
| | SLLTLMDGIGSEVSEGQVLVLGATNRPHALDAALRRPGRFDKEIEIGVPNAQDRLDIL | |
| | QKLLRRVPHLLTEAELLQLANSAHGYVGADLKVLCNEAGLCALRRILKKQPNLPDVKV | |
| | AGLVKITLKDFLQAMNDIRPSAMREIAIDVPNVSYDDVGGVRKQMAQIRELVELPLRH | |
| | PQLFKSIGIPAPRGLLLYGPPCTGKTMIARAVANEFGAYVSVINGPEIISKYVGESER | |
| | AVRQVFQRAKNSAPSIIFIDELDALCPKREGAQNEVEKRVVASLLTLMDGIGSVSIVL | |
| | VLGATNRPHALDAALRRPGRFDKEIEIGVPNAQDRLDILQKLLRRVPHLLTEAELLQL | |
| | ANSAHGYVGADLKVLCNEAGECGLLWDIQANLIMKRHFTQALSTVTPRIPESLRRFYE | |
| | DYQEKSGLHTL | |
| | SEQ ID NO:29 | 2789 bp |
| NOV12b, | CAGAGTTCGCCCTTCATTGAGTCGGCTTTTCTACTGCTTCGGCTAGGGTACCTTGTGA | |
| CG102575-02 | CCATGTCTTCCAAGAAGAATAGAAAGCGGTTGAACCAAAGCGCGGAAAATGGTTCGTC | |
| DNA Sequence | CTTGCCCTCTGCTGCTTCCTCTTGTGTGGAGGCACGGGCTCCTTCTGCTGGATCAGAC | |
| | TTCGCGGCAACCTCCGGGACTCTGACGGTGACCAACTTATTAGAAAAGGTAGATGACA | |
| | AAATTCCTAAAACATTCCAGAATTCCCTTATTCATCTTGGACTCAACACTATGAAGTC | |
| | TGCAAATATATGTATAGGTCGACCAGTGTTGCTTACTAGTTTGAACGGAAAGCAAGAG | |
| | GTGTATACAGCCTGGCCTATGGCAGGATTTCCTGGAGGCAAGGTCGGCCTGAGTGAAA | |
| | TGGCACAGAAAAATGTGGGTGTGAGGCCTGGTGATGCCATCCAGGTCCAGCCTCTTGT | |
| | GGGTGCTGTGCTACAGGCTGAGGAAATGGATGTGGCACTGAGTGACAAAGATATGGAA | |
| | ATTAATGAAGAAGAACTGACTGGTTGTATCCTGAGAAAACTAGATGGCAAGATTGTTT | |
| | TACCAGGCAACTTTCTGTATTGTACATTCTATGGACGACCGTACAAGCTGCAAGTATT | |
| | GCGAGTGAAAGGGGCAGATGGCATGATATTGGGAGGGCCTCAGAGTGACTCTGACACT | |
| | GATGCCCAAAGAATGGCCTTTGAACAGTCCAGCATGGAAACCAGTAGCCTGGAGTTAT | |
| | CCTTACAGCTAAGCCAGTTAGATCTGGAGGATACCCAGATCCCAACATCAAGAAGTAC | |
| | TCCTTATAAACCAATTGATGACAGAATTACAAATAAAGCCAGTGATGTTTTGCTGGAT | |
| | GTTACACAGAGCCCTGGAGATGGCAGTGGACTTATGCTAGAGGAAGTCACAGGTCTTA | |
| | AATGTAATTTTGAATCTGCCAGAGAAGGAAATGAGCAACTTACTGAAGAAGAGAGACT | |
| | GCTAAAGTTCAGCATAGGAGCAAAGTGCAATACTGATACTTTTTATTTTATTTCTTCA | |
| | ACAACAAGAGTCAATTTTACAGAGATTGATAAAAATTCAAAAGAGCAAGACAACCAAT | |
| | TTAAAGTAACTTATGACATGATAGGAGGATTAAGTAGCCAGCTGAAAGCAATTAGAGA | |
| | AATAATTGAATTGCCCCTCAAACAGCCTGAGCTTTTCAAGAGTTATGGAATTCCTGCC | |
| | CCTAGAGGAGTGTTACTTTATGGTCCTCCAGGTACTGGAAAAACAATGATCGCCAGGG | |
| | CTGTTGCTAATGAAGTTGGAGCCTATGTTTCTGTAATTAATGGTCCTGAAATTATAAG | |
| | CAAATTCTATGGTGAGACTGAAGCAAAGTTACGTCAGATATTTGCTGAAGCCACTCTA | |
| | CGACACCCATCAATTATTTTTATTGATGAGCTGGATGCACTTTGTCCGAAAAGAGAGG | |
| | GGGCCCAGAATGAAGTGGAAAAAAGAGTTGTGGCTTCACTCTTAACACTGATGGATGG | |
| | CATTGGTTCAGAAGTAAGTGAAGGACAAGTGTTGGTTCTTGGGGCCACAAATCGCCCT | |
| | CATGCCTTGGATGCTGCTCTCCGAAGACCTGGGCGATTTGATAAAGAGATTGAGATTG | |
| | GAGTTCCCAATGCTCAGGACCGGCTAGATATTCTCCAGAAACTGCTTCGAAGGGTACC | |
| | CCATTTGCTCACTGAGGCTGAGCTGCTGCAGCTGGCAAATAGTGCTCATGGATACGTT | |
| | GGAGCAGACTTGAAAGTCTTGTGTAATGAAGCAGGTCTCTGTGCCTTGCGGAGAATCC | |
| | TGAAAAAACAGCCTAACCTCCCTGATGTCAAGGTGGCTGGACTGGTGAAGATTACTCT | |
| | GAAGGATTTCTTGCAGGCAATGAATGATATCAGACCCAGTGCCATGAGGGAAATAGCA | |
| | ATTGATGTCCCAAATGTATCCTGGTCAGATATAGGAGGACTGGAAAGTATCAAACTGA | |
| | AGTTGGAACAGGCTGTGGAATGGCCCTTAAAACATCCAGAGTCTTTCATTCGAATGGG | |
| | TATTCAGCCACCTAAAGGAGTTCTTCTCTATGGGCCACCTGGGTGCTCTAAAACAATG | |
| | ATAGCAAAGGCTTTGGCCAATGAGAGTGGACTGAATTTTCTAGCTATAAAGGGGCCTG | |
| | AATTAATGAATAAATATGTTGGTGAATCTGAAAGAGCAGTTAGAGAGACCTTCCGAAA | |
| | AGCAAGAGCAGTGGCGCCTTCCATTATTTTCTTTGATGAACTGGATGCCTTAGCAGTT | |
| | GAAAGGGGCAGTTCTTTAGGTGCTGGGAATGTAGCCGATCGTGTTTTGGCTCAGCTCT | |
| | TAACAGAAATGGATGGGATTGAACAGCTAAAGGATGTGACCATTTTGGCAGCTACTAA | |
| | CCGTCCAGATAGGATAGACAAGGCTTTGATGCGGCCTGGAAGAATTGATAGAATCATC | |
| | TATGTGCCTTTACCGGATGCAGCAACAAGAAGGGAAATATTTAAGCTGCAGTTTCACT | |
| | CCATGCCTGTCAGTAATGAAGTTGACCTGGATGAACTCATCCTTCAAACCGACGCATA | |
| | CTCAGGAGCAGAGATTGTAGCTGTCTGCAGAGAGGCAGCTCTTCTGGCTCTGGAAGAA | |
| | GACATTCAAGCCAATCTCATCATGAAAAGACATTTCACTCAGGCCTTGAGCACTGTGA | |
| | CACCTAGAATTCCTGAGTCATTGAGACGTTTTTATGAAGATTATCAAGAGAAGAGTGG | |
| | GCTGCATACACTCTGAGAAAATATATATATTCAAGATGCTGAAAATCCTTTCCAGAGA | |
| | AAATT | |
| | ORF Start: ATG at 61 | ORF Stop: TGA at 2740 |
| | SEQ ID NO:30 | 893 aa MW at 97931.2 kD |

| | |
|---|---|
| NOV12b, | MSSKKNRKRLNQSAENGSSLPSAASSCVEARAPSAGSDFAATSGTLTVTNLLEKVDDK |
| CG102575-02 | IPKTFQNSLIHLGLNTMKSANICIGRPVLLTSLNGKQEVYTAWPMAGFPGGKVGLSEM |
| Protein Sequence | AQKNVGVRPGDAIQVQPLVGAVLQAEEMDVALSDKDMEINEEELTGCILRKLDGKIVL |
| | PGNFLYCTFYGRPYKLQVLRVKGADGMILGGPQSDSDTDAQRMAFEQSSMETSSLELS |
| | LQLSQLDLEDTQIPTSRSTPYKPIDDRITNKASDVLLDVTQSPGDGSGLMLEEVTGLK |
| | CNFESAREGNEQLTEEERLLKFSIGAKCNTDTFYFISSTTRVNFTEIDKNSKEQDNQF |
| | KVTYDMIGGLSSQLKAIREIIELPLKQPELFKSYGIPAPRGVLLYGPPGTGKTMIARA |
| | VANEVGAYVSVINGPEIISKFYGETEAKLRQIFAEATLRHPSIIFIDELDALCPKREG |
| | AQNEVEKRVVASLLTLMDGIGSEVSEGQVLVLGATNRPHALDAALRRPGRFDKEIEIG |
| | VPNAQDRLDILQKLLRRVPHLLTEAELLQLANSAHGYVGADLKVLCNEAGLCALRRIL |
| | KKQPNLPDVKVAGLVKITLKDFLQAMNDIRPSAMREIAIDVPNVSWSDIGGLESIKLK |
| | LEQAVEWPLKHPESFIRMGIQPPKGVLLYGPPGCSKTMIAKALANESGLNFLAIKGPE |
| | LMNKYVGESERAVRETFRKARAVAPSIIFFDELDALAVERGSSLGAGNVADRVLAQLL |
| | TEMDGIEQLKDVTILAATNRPDRIDKALMRPGRIDRIIYVPLPDAATRREIFKLQFHS |
| | MPVSNEVDLDELILQTDAYSGAEIVAVCREAALLALEEDIQANLIMKRHFTQALSTVT |
| | PRIPESLRRFYEDYQEKSGLHTL |

Sequence comparison of the above protein sequences yields the following sequence relationships shown in Table 12B.

TABLE 12B
Comparison of NOV12a against NOV12b.

| | | Identities/ |
|---|---|---|
| Protein | NOV12a Residues/ | Similarities for |
| Sequence | Match Residues | the Matched Region |
| NOV12b | 1 . . . 881 | 724/895 (80%) |
| | 1 . . . 893 | 764/895 (84%) |

Further analysis of the NOV12a protein yielded the following properties shown in Table 12C.

TABLE 12C
Protein Sequence Properties NOV12a

| | |
|---|---|
| PSort | 0.7000 probability located in plasma membrane; 0.3000 probability located in |
| analysis: | microbody (peroxisome); 0.2000 probability located in endoplasmic reticulum |
| | (membrane); 0.1000 probability located in mitochondrial inner membrane |
| SignalP | No Known Signal Sequence Predicted |
| analysis: | |

A search of the NOV12a protein against the Geneseq database, a proprietary database that contains sequences published in patents and patent publication, yielded several homologous proteins shown in Table 12D.

TABLE 12D
Geneseq Results for NOV12a

| | | NOV12a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAU17209 | Novel signal transduction pathway | 261 . . . 823 | 442/575 (76%) | 0.0 |
| | protein, Seq ID 774 - Homo sapiens, | 2 . . . 574 | 481/575 (82%) | |
| | 574 aa. [WO200154733-A1, 02 | | | |
| | AUG. 2001] | | | |
| AAB59399 | Protein tyrosine phosphatase related | 337 . . . 848 | 229/527 (43%) | e−120 |
| | sequence - Unidentified, 806 aa. | 190 . . . 711 | 340/527 (64%) | |
| | [WO200075339-A1, 14 DEC. 2000] | | | |
| AAE09327 | Human intracellular regulatory | 337 . . . 848 | 229/527 (43%) | e−120 |
| | molecule, VCP - Homo sapiens, 806 | 190 . . . 711 | 340/527 (64%) | |
| | aa. [US6274312-B1, 14 AUG. 2001] | | | |
| AAB05879 | Human transitional endoplasmic | 337 . . . 848 | 229/527 (43%) | e−120 |
| | reticulum ATPase protein sequence - | 190 . . . 711 | 340/527 (64%) | |
| | Homo sapiens, 806 aa. | | | |
| | [WO200034470-A1, 15 JUN. 2000] | | | |
| ABB59038 | Drosophila melanogaster | 322 . . . 844 | 228/540 (42%) | e−117 |
| | polypeptide SEQ ID NO 3906 - | 170 . . . 704 | 342/540 (63%) | |
| | Drosophila melanogaster, 801 aa. | | | |
| | [WO200171042-A2, 27 SEPT. 2001] | | | |

In a BLAST search of public sequence databases, the NOV12a protein was found to have homology to the proteins shown in the BLASTP data in Table 12E.

TABLE 12E
Public BLASTP Results for NOV12a

| | | NOV12a | Identities/ | |
|---|---|---|---|---|
| Protein | | Residues/ | Similarities for | |
| Accession | | Match | the Matched | Expect |
| Number | Protein/Organism/Length | Residues | Portion | Value |
| AAM00262 | SPERMATOGENESIS | 1 . . . 881 | 745/895 (83%) | 0.0 |
| | ASSOCIATED FACTOR - Homo | 1 . . . 893 | 785/895 (87%) | |
| | sapiens (Human), 893 aa. | | | |
| Q9Z2K7 | SPAF - Mus musculus (Mouse), | 1 . . . 881 | 640/895 (71%) | 0.0 |
| | 892 aa. | 1 . . . 892 | 721/895 (80%) | |
| Q9CXZ7 | 2510048F20RIK PROTEIN - Mus | 1 . . . 881 | 640/896 (71%) | 0.0 |
| | musculus (Mouse), 893 aa. | 1 . . . 893 | 721/896 (80%) | |
| Q8ZYN4 | AAA FAMILY ATPASE, | 356 . . . 876 | 265/537 (49%) | e−136 |
| | POSSIBLE CELL DIVISION | 184 . . . 714 | 358/537 (66%) | |
| | CONTROL PROTEIN CDC48 - | | | |
| | Pyrobaculum aerophilum, 731 aa. | | | |
| Q58556 | Cell division cycle protein 48 | 309 . . . 855 | 271/578 (46%) | e−136 |
| | homolog MJ1156 - Methanococcus | 127 . . . 697 | 378/578 (64%) | |
| | jannaschii, 903 aa. | | | |

PFam analysis predicts that the NOV12a protein contains the domains shown in the Table 12F.

TABLE 12F
Domain Analysis of NOV12a

| | | Identities/ | |
|---|---|---|---|
| Pfam | | Similarities | Expect |
| Domain | NOV12a Match Region | for the Matched Region | Value |
| AAA | 378 . . . 566 | 95/217 (44%) | 3.3e−75 |
| | | 165/217 (76%) | |
| AAA | 652 . . . 837 | 98/217 (45%) | 2e−77 |
| | | 165/217 (76%) | |

### Example 13

The NOV13 clone was analyzed, and the nucleotide and encoded polypeptide sequences are shown in Table 13A.

TABLE 13A
NOV13 Sequence Analysis

| | SEQ ID NO:31 | 420 bp |
|---|---|---|
| NOV13a, | TGCAGAAGGTGACCCTGGGCCTGCTTGTGTTCCTGGCAGGCTTTCCTGTCCTGGACGC | |
| CG102615-01 | CAATGACCTAGAAGATAAAAACAGTCCTTTCTACTATGACTGGCACAGCCTCCAGGTT | |
| DNA Sequence | GGCGGGCTCATCTGCGCTGGGGTTCTGTGCGCCATGGGCATCATCATCGTCATGAGTG | |
| | CAAAATGCAAATGCAAGTTTGGCCAGAAGTCCGGTCACCATCCAGGGGAGACTCCACC | |
| | TCTCATCACCCCAGGCTCAGCCCAAAGCTGATGAGGACAGACCAGCTGAAATTGGGTG | |
| | GAGGACCGTTCTCTGTCCCCAGGTCCTGTCTCTGCACAGAAACTTGAACTCCAGGATG | |
| | GAATTCTTCCTCCTCTGCTGGGACTCCTTTGCATGGCAGGGCCTCATCTCACCTCTCG | |
| | CAAGAGGGTCTCTT | |
| | ORF Start: at 3 | ORF Stop: TGA at 261 |
| | SEQ ID NO:32 | 86 aa MW at 9131.6 kD |
| NOV13a, | QKVTLGLLVFLAGFPVLDANDLEDKNSPFYYDWHSLQVGGLICAGVLCAMGIIIVMSA | |
| CG102615-01 | KCKCKFGQKSGHHPGETPPLITPGSAQS | |
| Protein Sequence | | |
| | SEQ ID NO:33 | 462 bp |
| NOV13b, | TCAGCCTGGTGAACCACACAGAGGCTGGGGCGAGGAGGATACCATCTGTCAGTCTTGG | |
| CG102615-04 | CTGGATGACATCATGGGAAGGGGGTATAGTGGGGCCTTGCAGGCCAGAGGTGGCTTGG | |
| DNA Sequence | AGGAGCCCCTGGAAAGAGGCTTAAGAGGCCAGCGCTCTGACATGCAGAAGGTGACCCT | |
| | GGGCCTGCTTGTGTTCCTGGCAGGCTTTCCTGTCCTGGACGCCAATGACCTAGAAGAT | |
| | AAAAACAGTCCTTTCTACTATGACTGGCACAGCCTCCAGGTTGGCGGGCTCATCTGCG | |
| | CTGGGGTTCTGTGCGCCATGGGCATCATCATCGTCATGAGTGCAAAATGCAAATGCAA | |
| | GTTTGGCCAGAAGTCCGGTCACCATCCAGGGGAGACTCCACCTCTCATCACCCCAGGC | |
| | TCAGCCCAAAGCTGATGAGGACAGACCAGCTGAAATTGGGTGGAGGACCGTTCTCT | |
| | ORF Start: ATG at 71 | ORF Stop: TGA at 419 |
| | SEQ ID NO:34 | 116 aa MW at 12362.2 kD |

| | |
|---|---|
| NOV13b, | MGRGYSGALQARGGLEEPLERGLRGQRSDMQKVTLGLLVFLAGFPVLDANDLEDKNSP |
| CG102615-04 | FYYDWHSLQVGGLICAGVLCAMGIIIVMSAKCKCKFGQKSGHHPGETPPLITPGSAQS |
| Protein Sequence | |

Sequence comparison of the above protein sequences yields the following sequence relationships shown in Table 13B.

TABLE 13B
Comparison of NOV13a against NOV13b.

| | NOV13a Residues/ | Identities/Similarities |
|---|---|---|
| Protein Sequence | Match Residues | for the Matched Region |
| NOV13b | 1 . . . 86 | 86/86 (100%) |
| | 31 . . . 116 | 86/86 (100%) |

Further analysis of the NOV13a protein yielded the following properties shown in Table 13C.

TABLE 13C
Protein Sequence Properties NOV13a

| | |
|---|---|
| PSort | 0.4600 probability located in plasma membrane; |
| analysis: | 0.2000 probability located in |
| | lysosome (membrane); 0.1000 probability |
| | located in endoplasmic reticulum |
| | (membrane); 0.1000 probability located |
| | in endoplasmic reticulum (lumen) |
| SignalP | Cleavage site between residues 20 and 21 |
| analysis: | |

A search of the NOV13a protein against the Geneseq database, a proprietary database that contains sequences published in patents and patent publication, yielded several homologous proteins shown in Table 13D.

TABLE 13D
Geneseq Results for NOV13a

| | | NOV13a | Identities/ | |
|---|---|---|---|---|
| | | Residues/ | Similarities for | |
| Geneseq | Protein/Organism/Length [Patent | Match | the Matched | Expect |
| Identifier | #, Date] | Residues | Region | Value |
| AAM23962 | Human EST encoded protein SEQ | 1 . . . 86 | 86/86 (100%) | 7e−47 |
| | ID NO: 1487 - Homo sapiens, 87 aa. | 2 . . . 87 | 86/86 (100%) | |
| | [WO200154477-A2, 02-AUG-2001] | | | |
| AAW92959 | Human MAT-8 protein - Homo | 1 . . . 86 | 86/86 (100%) | 7e−47 |
| | sapiens, 87 aa. [WO9905276-A1, | 2 . . . 87 | 86/86 (100%) | |
| | 04-FEB-1999] | | | |
| AAY48304 | Human prostate cancer-associated | 1 . . . 86 | 86/86 (100%) | 7e−47 |
| | protein 1 - Homo sapiens, 87 aa. | 2 . . . 87 | 86/86 (100%) | |
| | [DE19811194-A1, 16-SEP-1999] | | | |
| AAR90990 | Human Mat-8 polypeptide - Homo | 1 . . . 86 | 86/86 (100%) | 7e−47 |
| | sapiens, 87 aa. [WO9605322-A1, | 2 . . . 87 | 86/86 (100%) | |
| | 22-FEB-1996] | | | |
| AAB53415 | Human colon cancer antigen protein | 1 . . . 86 | 86/112 (76%) | 2e−42 |
| | sequence SEQ ID NO: 955 - Homo | 39 . . . 150 | 86/112 (76%) | |
| | sapiens, 150 aa. [WO200055351-A1, | | | |
| | 21-SEP-2000] | | | |

In a BLAST search of public sequence databases, the NOV13a protein was found to have homology to the proteins shown in the BLASTP data in Table 13E.

TABLE 13E
Public BLASTP Results for NOV13a

| | | | Identities/ | |
|---|---|---|---|---|
| | | NOV13a | Similarities | |
| Protein | | Residues/ | for the | |
| Accession | | Match | Matched | Expect |
| Number | Protein/Organism/Length | Residues | Portion | Value |
| Q14802 | FXYD domain-containing ion transport | 1 . . . 86 | 86/86 (100%) | 2e−46 |
| | regulator 3 precursor (Chloride | 2 . . . 87 | 86/86 (100%) | |
| | conductance inducer protein Mat-8) | | | |
| | (Mammary tumor 8 kDa protein) | | | |
| | (Phospholemman-like) - Homo sapiens | | | |
| | (Human), 87 aa. | | | |
| Q61835 | FXYD domain-containing ion transport | 1 . . . 86 | 63/86 (73%) | 2e−33 |
| | regulator 3 precursor (Chloride | 2 . . . 87 | 72/86 (83%) | |
| | conductance inducer protein Mat-8) | | | |
| | (Mammary tumor 8 kDa protein) | | | |
| | (Phospholemman-like) - Mus musculus | | | |
| | (Mouse), 88 aa. | | | |
| O97797 | FXYD domain-containing ion transport | 2 . . . 84 | 60/83 (72%) | 8e−32 |
| | regulator 3 precursor (Chloride | 3 . . . 85 | 68/83 (81%) | |
| | conductance inducer protein Mat-8) | | | |
| | (Mammary tumor 8 kDa protein) - Sus | | | |
| | scrofa (Pig), 88 aa. | | | |
| Q9D2W0 | FXYD domain-containing ion transport | 1 . . . 86 | 45/86 (52%) | 4e−21 |
| | regulator 4 precursor (Channel | 2 . . . 87 | 59/86 (68%) | |
| | inducing factor) (CHIF) - Mus | | | |
| | musculus (Mouse), 88 aa. | | | |
| Q63113 | FXYD domain-containing ion transport | 3 . . . 86 | 44/84 (52%) | 7e−20 |
| | regulator 4 precursor (Channel | 4 . . . 87 | 55/84 (65%) | |
| | inducing factor) (CHIF) | | | |
| | (Corticosteroid-induced protein) - | | | |
| | Rattus norvegicus (Rat), 87 aa. | | | |

PFam analysis predicts that the NOV13a protein contains the domains shown in the Table 13F.

TABLE 13F
Domain Analysis of NOV13a

| | | Identities/ | |
|---|---|---|---|
| | | Similarities | |
| | NOV13a Match | for the Matched | Expect |
| Pfam Domain | Region | Region | Value |
| ATP1G1_PLM_MAT8 | 19 . . . 74 | 27/57 (47%) | 2.7e−35 |
| | | 55/57 (96%) | |

### Example 14

The NOV14 clone was analyzed, and the nucleotide and encoded polypeptide sequences are shown in Table 14A.

TABLE 14A
NOV14 Sequence Analysis

| | SEQ ID NO:35 | 1638 bp |
|---|---|---|
| NOV14a, | TTATCTAATATGTTTGTTTTAGCTACATCTTTATCAAGCCAAGTGAATCCTGACTGGC | |
| CG102646-01 | GAACATATATCATGCTTGCAGTATATTTTCTAATTTTACTTGTTATTGGATATTATGG | |
| DNA Sequence | TTATAAGCAAGCAACCGGAGATTTAAGTGAATATATGCTTGGCGAAAGAAATATTGGT | |
| | CCATATGTCACTGCCTTATCTGCCGGAGCTTCAGATATGAGCGGTTGGATGATTATGG | |
| | GATTACCTGGAGAAGTTTATACTACAGGTTTATCAGCAGCATGGTTAGCTATTGGGTT | |
| | AACTATCGGAGCTTATGTTAACTACATACTTGTAGCACCAAGACTTCGTGTGTACACT | |
| | GAAAAAGCCAATGACTCAATTACATTGCCTAATTACTTTACACATCGTCTTAATGATA | |
| | ATTCCAATATTATTAAAATTATCTCTGGTGGTATCATTGTTGTATTTTTTACACTCTA | |
| | TACTCATTCAGGTATGGTATCAGGTGGTAAATTATTTGATAGTGCTTTTGGTTTAGAC | |
| | TATCATATTGGACTTATTTTAATCTCTGTCATTGTAATTTTATATACTTTTTTTGGTG | |
| | GCTATTTAGCAGTGTCGTTAACTGACTTTTTCCAAGGGGTTGTCATGTTAATTGCGAT | |
| | GGTTATGGTACCTATTGTAGCCATGATGCAGCTCGGAGGTATGGATGCTTTTTCACAA | |
| | GCAGCAACATTAAAACCTACTAATTTAGATTTATTTAAAGGAACAACTATTATAGGCA | |
| | TCATTTCATTCTTTGCTTGGGGATTAGGCTATTTTGGCCAGCCTCATATCATTGTACG | |
| | ATTTATGTCTATCAAATCCGTACGACAATTAAAAACGTCTAGAAGATTTGGTATTAGT | |
| | TGGATGGCTATTAGTTTAATCGGTGCAGTATGTGTTGGATTAATTGGCATTTCGTTTG | |
| | TACAAGATAAAGGTGTTGAATTAAAAGATCCAGAAACACTATTTATTTTAATGGGACA | |
| | AATTTTATTCCATCCTCTTGTAGGTGGGTTCCTACTTGCAGCCATTTTGGCAGCAATT | |
| | ATGAGTACGATTTCTTCCCAATTACTTGTGACTTCAAGTTCACTTACAGAAGATTTTT | |
| | ACAAGTTAATTCGTGGTGAAGAAGCAGCAAAGCAACATAAGAAAGAATTTTTATTAGT | |
| | GGGTCGATTATCTGTTGTAGTCGTTGCGATTATCTCCATCCTCATTGCATGGACGCCA | |
| | AATGACACTATCTTAAATCTTGTTGGTAACGCTTGGGCTGGATTCGGTGCAGCATTTG | |
| | GTCCACTGGTATTATTATCTCTCTATTCGAAAGGTTTAAGTCGTACTGGAGCTATTTC | |
| | TGGAATGTTATCAGGAGCAATTGTCGTCATTCTTTGGATTGTGTTTGTTAAACCATTA | |
| | GGAGCATATAATGATTTCTTTAATTTATATGAAATTATTCCTGGTTTCTTAACAAGTC | |
| | TTATTGTGACATATGTAGTGAGTCTTGTAACTAAAAAGCCAGATCTCAATGTTCAAAA | |
| | AGATTTAGAAGACGTCAAACGTATTGTAAAAGGACAATAAATTAATAATATTCAACGA | |
| | TGCTTAATGTCAATATTATTTCAATTAGTGCATTACTCTTATAATATGAAACACAAAT | |
| | AAATTTTTATACAT | |
| | ORF Start: ATG at 10 | ORF Stop: TAA at 1546 |
| | SEQ ID NO:36 | 512 aa MW at 55813.4 kD |

| | |
|---|---|
| NOV14a, | MFVLATSLSSQVNPDWRTYIMLAVYFLILLVIGYYGYKQATGDLSEYMLGERNIGPYV |
| CG102646-01 | TALSAGASDMSGWMIMGLPGEVYTTGLSAAWLAIGLTIGAYVNYILVAPRLRVYTEKA |
| Protein Sequence | NDSITLPNYFTHRLNDNSNIIKIISGGIIVVFFTLYTHSGMVSGGKLFDSAFGLDYHI |
| | GLILISVIVILYTFFGGYLAVSLTDFFQGVVMLIAMVMVPIVAMMQLGGMDAFSQAAT |
| | LKPTNLDLFKGTTIIGIISFFAWGLGYFGQPHIIVRFMSIKSVRQLKTSRRFGISWMA |
| | ISLIGAVCVGLIGISFVQDKGVELKDPETLFILMGQILFHPLVGGFLLAAILAAIMST |
| | ISSQLLVTSSSLTEDFYKLIRGEEAAKQHKKEFLLVGRLSVVVVAIISILIAWTPNDT |
| | ILNLVGNAWAGFGAAFGPLVLLSLYSKGLSRTGAISGMLSGAIVVILWIVFVKPLGAY |
| | NDFFNLYEIIPGFLTSLIVTYVVSLVTKKPDLNVQKDLEDVKRIVKGQ |

Further analysis of the NOV14a protein yielded the following properties shown in Table 14B.

TABLE 14B
Protein Sequence Properties NOV14a

| | |
|---|---|
| PSort | 0.8200 probability located in plasma membrane; |
| analysis: | 0.4600 probability located in |
| | Golgi body; 0.3700 probability located in |
| | endoplasmic reticulum (membrane); |
| | 0.1000 probability located in endoplasmic |
| | reticulum (lumen) |
| SignalP | Cleavage site between residues 37 and 38 |
| analysis: | |

A search of the NOV14a protein against the Geneseq database, a proprietary database that contains sequences published in patents and patent publication, yielded several homologous proteins shown in Table 14C.

TABLE 14C
Geneseq Results for NOV14a

| | | NOV14a | Identities/ | |
|---|---|---|---|---|
| | | Residues/ | Similarities for | |
| Geneseq | Protein/Organism/Length [Patent | Match | the Matched | Expect |
| Identifier | #, Date] | Residues | Region | Value |
| AAB76757 | Corynebacterium glutamicum MCT | 20 . . . 506 | 233/502 (46%) | e−127 |
| | protein SEQ ID NO: 496 - | 9 . . . 499 | 339/502 (67%) | |
| | Corynebacterium glutamicum, 524 | | | |
| | aa. [WO200100805-A2, 04-JAN-2001] | | | |
| AAG93195 | C glutamicum protein fragment SEQ | 20 . . . 506 | 233/502 (46%) | e−127 |
| | ID NO: 6949 - Corynebacterium | 9 . . . 499 | 339/502 (67%) | |
| | glutamicum, 524 aa. [EP1108790- | | | |
| | A2, 20-JUN-2001] | | | |
| AAW20806 | H. pylori transporter protein, | 64 . . . 506 | 208/450 (46%) | e−112 |
| | 09ap20802orf27 - Helicobacter | 5 . . . 445 | 306/450 (67%) | |
| | pylori, 446 aa. [WO9640893-A1, | | | |
| | 19-DEC-1996] | | | |
| AAG82596 | S. epidermidis open reading frame | 266 . . . 510 | 171/245 (69%) | 1e−94 |
| | protein sequence SEQ ID NO: 2286 - | 163 . . . 407 | 208/245 (84%) | |
| | Staphylococcus epidermidis, 408 aa. | | | |
| | [WO200134809-A2, 17-MAY-2001] | | | |
| AAB96626 | Putative P. abyssi permease #22 - | 24 . . . 508 | 174/503 (34%) | 4e−83 |
| | Pyrococcus abyssi, 537 aa. | 11 . . . 507 | 275/503 (54%) | |
| | [FR2792651-A1, 27-OCT-2000] | | | |

In a BLAST search of public sequence databases, the NOV14a protein was found to have homology to the proteins shown in the BLASTP data in Table 14D.

TABLE 14D
Public BLASTP Results for NOV14a

| | | NOV14a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q99SY5 | HIGH AFFINITY PROLINE | 1 . . . 510 | 378/510 (74%) | 0.0 |
| | PERMEASE - Staphylococcus | 1 . . . 510 | 443/510 (86%) | |
| | aureus (strain Mu50/ATCC | | | |
| | 700699), and, 512 aa. | | | |
| O30986 | HIGH AFFINITY PROLINE | 1 . . . 494 | 366/494 (74%) | 0.0 |
| | PERMEASE - Staphylococcus | 1 . . . 493 | 431/494 (87%) | |
| | aureus, 497 aa. | | | |
| Q53584 | PROLINE PERMEASE | 1 . . . 494 | 366/494 (74%) | 0.0 |
| | HOMOLOG - Staphylococcus | 1 . . . 493 | 430/494 (86%) | |
| | aureus, 497 aa. | | | |
| O06493 | Osmoregulated proline transporter | 20 . . . 494 | 268/478 (56%) | e−158 |
| | (Sodium/proline symporter) - | 7 . . . 473 | 371/478 (77%) | |
| | Bacillus subtilis, 492 aa. | | | |
| P94392 | HOMOLOGUE OF PROLINE | 54 . . . 504 | 243/452 (53%) | e−142 |
| | PERMEASE OF E. COLI - Bacillus | 1 . . . 442 | 336/452 (73%) | |
| | subtilis, 449 aa. | | | |

PFam analysis predicts that the NOV14a protein contains the domains shown in the Table 14E.

TABLE 14E
Domain Analysis of NOV14a

| | | Identities/ | |
|---|---|---|---|
| | | Similarities | |
| | NOV14a | for the | |
| Pfam Domain | Match Region | Matched Region | Expect Value |
| SSF | 47 . . . 447 | 134/449 (30%) | 5.7e−121 |
| | | 318/449 (71%) | |

### Example 15

The NOV15 clone was analyzed, and the nucleotide and encoded polypeptide sequences are shown in Table 15A.

TABLE 15A
NOV15 Sequence Analysis

| | SEQ ID NO:37 | 1146 bp |
|---|---|---|
| NOV15a, | CTAGCTCGACAGCTTCCCGGCGGCTGCGCGATGGACAGCCCCGAGGTGACCTTCACTC | |
| CG102878-01 | TCGCCTATCTGGTGTTCGCCGTGTGCTTCGTGTTCACGCCCAACGAGTTCCACGCGGC | |
| DNA Sequence | GGGGCTCACGGTGCAGAACCTGCTGTCGGGCTGGCTGGGCAGCGAGGACGCCGCCTTC | |
| | GTGCCCTTCCACTTGCGCCGCACGGCCGCCACGCTGTTGTGCCACTCGCTGCTGCCGC | |
| | TCGGCTACTATGTGGGCATGTGCCTTGCGGCTTCAGAAAAGCGGCTCCACGCCCTCAG | |
| | CCAGGCCCCTGAGGCCTGGCGGCTCTTCCTGCTGCTGGCCGTGACCCTCCCCTCCATC | |
| | GCCTGCATCCTGATCTACTACTGGTCCCGTGACCGGTGGGCCTGCCACCCACTGGCGC | |
| | GCACCCTGGCCCTCTACGCCCTCCCACAGTCTGGCTGGCAGGCTGTTGCCTCCTCTGT | |
| | CAACACTGAGTTCCGGCGGATTGACAAGTTTGCCACCGGTGCACCAGGTGCCCGTGTG | |
| | ATTGTGACAGACACGTGGGTGATGAAGGTAACCACCTACCGAGTGCACGTGGCCCAGC | |
| | AGCAGGACGTGCACCTGACTGTGACGGAGTCTCGGCAGCATGAGCTCTCGCCAGACTC | |
| | GAACTTGCCCGTGCAGCTCCTCACCATCCGTGTGGCCAGCACCAACCCTGCTGTGCAG | |
| | GCCTTTGACATCAGGCTGAACTCCACTGAGTACGGGGAGCTCTGCGAGAAGCTCCGGG | |
| | CACCCATCCGCAGGGCAGCCCATGTGGTCATCCACCAGAGCCTGGGCGACCTGTTCCT | |
| | GGAGACATTTGCCTCCCTGGTAGAGGTCAACCCGGCCTACTCAGTGCCCAGCAGCCAG | |
| | GAGCTGGAGGCCTGCATAGGCTGCATGCAGACACGTGCCAGCGTGAAGCTGGTGAAGA | |
| | CCTGCCAGGAGGCAGCCACAGGCGAGTGCCAGCAGTGTTACTGCCGCCCCATGTGGTG | |
| | CCTCACCTGCATGGGCAAGTGGTTCGCCAGCCGCCAGGACCCCCTGCGCCCTGACACC | |
| | TGGCTGGCCAGCCGCGTGCCCTGCCCCACCTGCCGCGCACGCTTCTGCATCCTGGATG | |
| | TGTGCACCGTGCGCTGAGTGGGCTGGGGCCTTGAGGTGACTCTG | |
| | ORF Start: ATG at 31 | ORF Stop: TGA at 1117 |
| | SEQ ID NO:38 | 362 aa MW at 40433.3 kD |
| NOV15a, | MDSPEVTFTLAYLVFAVCFVFTPNEFHAAGLTVQNLLSGWLGSEDAAFVPFHLRRTAA | |
| CG102878-01 | TLLCHSLLPLGYYVGMCLAASEKRLHALSQAPEAWRLFLLLAVTLPSIACILIYYWSR | |
| Protein Sequence | DRWACHPLARTLALYALPQSGWQAVASSVNTEFRRIDKFATGAPGARVIVTDTWVMKV | |
| | TTYRVHVAQQQDVHLTVTESRQHELSPDSNLPVQLLTIRVASTNPAVQAFDIRLNSTE | |
| | YGELCEKLRAPIRRAAHVVIHQSLGDLFLETFASLVEVNPAYSVPSSQELEACIGCMQ | |
| | TRASVKLVKTCQEAATGECQQCYCRPMWCLTCMGKWFASRQDPLRPDTWLASRVPCPT | |
| | CRARFCILDVCTVR | |
| | SEQ ID NO:39 | 1115 bp |
| NOV15b, | TTCGCCCTTGGCTGCGCGATGGACAGCCCCGAGGTGACCTTCACTCTCGCCTATCTGG | |
| CG102878-02 | TGTTCGCCGTGTGCTTCGTGTTCACGCCCAACGAGTTCCACGCGGCGGGGCTCACGGT | |
| DNA Sequence | GCAGAACCTGCTGTCGGGCTGGCTGGGCAGCGAGGACGCCGCCTTCGTGCCCTTCCAC | |
| | TTGCGCCGCACGGCCGCCACGCTGTTGTGCCACTCGCTGCTGCCGCTCGGCTACTACG | |
| | TGGGCATGTGCCTTGCGGCTTCAGAAAAGCGGCTCCACGCCCTCAGCCAGGCCCCTGA | |
| | GGCCTGGCGGCTCTTCCTGCTGCTGGCCGTGACCCTCCCCTCCATTGCCTGCATCCTG | |
| | ATCTACTACTGGTCCCGTGACCGGTGGGCCTGCCACCCACTGGCGCGCACCCTGGCCC | |
| | TCTACGCCCTCCCACAGTCTGGCTGGCAGGCTGTTGCCTCCTCTGTCAACACTGAGTT | |
| | CCGGCGGATTGACAAGTTTGCCACCGGTGCACCAGGTGCCCGTGTGATTGTGACAGAC | |
| | ACGTGGGTGATGAAGGTAACCACCTACCGAGTGCACGTGGCCCAGCAGCAGGACGTGC | |
| | ACCTGACTGTGACGGAGTCTCGGCAGCATGAGCTCTCGCCAGACTCGAACTTGCCCGT | |
| | GCAGCTCCTCACCATCCGTGTGGCCAGCACCAACCCTGCTGTGCAGGCCTTTGACATC | |
| | TGGCTGAACTCCACTGAGTACGGGGAGCTCTGCGAGAAGCTCCGGGCACCCATCCGCA | |
| | GGGCAGCCCATGTGGTCATCCACCAGAGCCTGGGCGACCTGTTCCTGGAGACATTTGC | |
| | CTCCCTGGTAGAGGTCAACCCGGCCTACTCAGTGCCCAGCAGCCAGGAGCTGGAGGCC | |
| | TGCATAGGCTGCATGCAGACACGTGCCAGCGTGAAGCTGGTGAAGACCTGCCAGGAGG | |
| | CAGCCACAGGCGAGTGCCAGCAGTGTTACTGCCGCCCCATGTGGTGCCTCACCTGCAT | |
| | GGGCAAGTGGTTCGCCAGCCGCCAGGACCCCCTGCGCCCTGACACCTGGCTGGCCAGC | |
| | CGCGTGCCCTGCCCCACCTGCCGCGCACGCTTCTGCATCCTGGATGTGTGCACCGTGC | |
| | GCTGATGTGGCGG | |
| | ORF Start: ATG at 19 | ORF Stop: TGA at 1105 |
| | SEQ ID NO: 40 | 362 aa MW at 40463.4 kD |

| | |
|---|---|
| NOV15b, | MDSPEVTFTLAYLVFAVCFVFTPNEFHAAGLTVQNLLSGWLGSEDAAFVPFHLRRTAA |
| CG102878-02 | TLLCHSLLPLGYYVGMCLAASEKRLHALSQAPEAWRLFLLLAVTLPSIACILIYYWSR |
| Protein Sequence | DRWACHPLARTLALYALPQSGWQAVASSVNTEFRRIDKFATGAPGARVIVTDTWVMKV |
| | TTYRVHVAQQQDVHLTVTESRQHELSPDSNLPVQLLTIRVASTNPAVQAFDIWLNSTE |
| | YGELCEKLRAPIRRAAHVVIHQSLGDLFLETFASLVEVNPAYSVPSSQELEACIGCMQ |
| | TRASVKLVKTCQEAATGECQQCYCRPMWCLTCMGKWFASRQDPLRPDTWLASRVPCPT |
| | CRARFCILDVCTVR |

Sequence comparison of the above protein sequences yields the following sequence relationships shown in Table 15B.

TABLE 15B
Comparison of NOV15a against NOV15b.

| | NOV15a Residues/ | Identities/Similarities |
|---|---|---|
| Protein Sequence | Match Residues | for the Matched Region |
| NOV15b | 1 . . . 362 | 361/362 (99%) |
| | 1 . . . 362 | 361/362 (99%) |

Further analysis of the NOV15a protein yielded the following properties shown in Table 15C.

TABLE 15C
Protein Sequence Properties NOV15a

| | |
|---|---|
| PSort | 0.6760 probability located in plasma membrane; |
| analysis: | 0.1000 probability located in |
| | endoplasmic reticulum (membrane); |
| | 0.1000 probability located in |
| | endoplasmic reticulum (lumen); |
| | 0.1000 probability located in outside |
| SignalP | Cleavage site between residues 29 and 30 |
| analysis: | |

A search of the NOV15a protein against the Geneseq database, a proprietary database that contains sequences published in patents and patent publication, yielded several homologous proteins shown in Table 15D.

TABLE 15D
Geneseq Results for NOV15a

| | | NOV15a | Identities/ | |
|---|---|---|---|---|
| | | Residues/ | Similarities for | |
| Geneseq | Protein/Organism/Length [Patent | Match | the Matched | Expect |
| Identifier | #, Date] | Residues | Region | Value |
| AAG81377 | Human AFP protein sequence SEQ | 1 . . . 362 | 360/362 (99%) | 0.0 |
| | ID NO: 272 - Homo sapiens, 362 aa. | 1 . . . 362 | 360/362 (99%) | |
| | [WO200129221-A2, 26-APR-2001] | | | |
| ABB69639 | Drosophila melanogaster | 1 . . . 358 | 122/389 (31%) | 7e−60 |
| | polypeptide SEQ ID NO: 35709 - | 1 . . . 383 | 200/389 (51%) | |
| | Drosophila melanogaster, 409 aa. | | | |
| | [WO200171042-A2, 27-SEP-2001] | | | |
| AAG23427 | Arabidopsis thaliana protein | 337 . . . 362 | 13/26 (50%) | 2.8 |
| | fragment SEQ ID NO: 26729 - | 77 . . . 102 | 16/26 (61%) | |
| | Arabidopsis thaliana, 284 aa. | | | |
| | [EP1033405-A2, 06-SEP-2000] | | | |
| AAG23426 | Arabidopsis thaliana protein | 337 . . . 362 | 13/26 (50%) | 2.8 |
| | fragment SEQ ID NO: 26728 - | 206 . . . 231 | 16/26 (61%) | |
| | Arabidopsis thaliana, 413 aa. | | | |
| | [EP1033405-A2, 06-SEP-2000] | | | |
| ABG11786 | Novel human diagnostic protein | 285 . . . 354 | 23/89 (25%) | 3.6 |
| | #11777 - Homo sapiens, 198 aa. | 54 . . . 141 | 37/89 (40%) | |
| | [WO200175067-A2, 11-OCT-2001] | | | |

In a BLAST search of public sequence databases, the NOV15a protein was found to have homology to the proteins shown in the BLASTP data in Table 15E.

TABLE 15E
Public BLASTP Results for NOV15a

| | | NOV15a | Identities/ | |
|---|---|---|---|---|
| Protein | | Residues/ | Similarities for | |
| Accession | | Match | the Matched | Expect |
| Number | Protein/Organism/Length | Residues | Portion | Value |
| CAC38627 | SEQUENCE 271 FROM | 1 . . . 362 | 361/362 (99%) | 0.0 |
| | PATENT WO0129221 - Homo | 1 . . . 362 | 361/362 (99%) | |
| | sapiens (Human), 362 aa. | | | |
| Q9DCF3 | 0610039G24RIK PROTEIN - | 1 . . . 362 | 323/362 (89%) | 0.0 |
| | Mus musculus (Mouse), 362 aa. | 1 . . . 362 | 341/362 (93%) | |
| Q96GP5 | SIMILAR TO RIKEN CDNA | 1 . . . 226 | 226/226 (100%) | e−129 |
| | 0610039G24 GENE - Homo | 1 . . . 226 | 226/226 (100%) | |
| | sapiens (Human), 232 aa. | | | |
| Q9VN16 | CG14646 PROTEIN - Drosophila | 1 . . . 358 | 122/389 (31%) | 2e−59 |
| | melanogaster (Fruit fly), 409 aa. | 1 . . . 383 | 200/389 (51%) | |
| Q95TM4 | LD39811P - Drosophila | 20 . . . 358 | 116/370 (31%) | 1e−55 |
| | melanogaster (Fruit fly), 393 aa. | 4 . . . 367 | 190/370 (51%) | |

### Example 16

The NOV16 clone was analyzed, and the nucleotide and encoded polypeptide sequences are shown in Table 16A.

TABLE 16A
NOV16 Sequence Analysis

| | SEQ ID NO:41 | 2765 bp |
|---|---|---|
| NOV 16a, | CTGGCGGCGTCGCATGGAGGGCTCTGGGGGCGGTGCGGGCGAGCGGGCGCCGCTGCTG | |
| CG103459-01 | GGCGCGCGGCGGGCGGCGGCGGCCGCGGCGGCGGCTGGGGCGTTCGCGGGCCGGCGCG | |
| DNA Sequence | CGGCGTGCGGGGCCGTGCTGCTGACGGAGCTGCTGGAGCGCGCCGCTTTCTACGGCAT | |
| | CACGTCCAACCTGGTGCTATTCCTGAACGGGGCGCCGTTCTGCTGGGAGGGCGCGCAG | |
| | GCCAGCGAGGCGCTGCTGCTCTTCATGGGCCTCACCTACCTGGGCTCGCCGTTCGGAG | |
| | GCTGGCTGGCCGACGCGCGGCTGGGCCGGGCGCGCGCCATCCTGCTGAGCCTGGCGCT | |
| | CTACCTGCTGGGCATGCTGGCCTTCCCGCTGCTGGCCGCGCCCGCCACGCGAGCCGCG | |
| | CTCTGCGGTTCCGCGCGCCTGCTCAACTGCACGGCGCCTGGTCCCGACGCCGCCGCCC | |
| | GCTGCTGCTCACCGGCCACCTTCGCGGGGCTGGTGCTGGTGGGCCTGGGCGTGGCCAC | |
| | CGTCAAGGCCAACATCACGCCCTTCGGCGCCGACCAGGTTAAAGATCGAGGTCCGGAA | |
| | GCCACTAGGAGATTTTTTAATTGGTTTTATTGGAGCATTAACCTGGGAGCGATCCTGT | |
| | CGTTAGGTGGCATTGCCTATATTCAGCAGAACGTCAGCTTTGTCACTGGTTATGCGAT | |
| | CCCCACTGTCTGCGTCGGCCTTGCTTTTGTGGCCTTCCTCTGTGGCCAGAGCGTTTTC | |
| | ATCACCAAGCCTCCTGATGGCAGTGCCTTCACCGATATGTTCAAGATACTGACGTATT | |
| | CCTGCTGTTCCCAGAAGCGAAGTGGAGAGCGCCAGAGTAATGGTGAAGGCATTGGAGT | |
| | CTTTCAGCAATCTTCTAAACAAAGTCTGTTTGATTCATGTAAGATGTCTCATGGTGGG | |
| | CCATTTACAGAAGAGAAAGTGGAAGATGTGAAAGCTCTGGTCAAGATTGTCCCTGTTT | |
| | TCTTGGCTTTGATACCTTACTGGACAGTGTATTTCCAAATGCAGACAACATATGTTTT | |
| | ACAGAGTCTTCATTTGAGGATTCCAGAAATTTCAAATATTACAACCACTCCTCACACG | |
| | CTCCCTGCAGCCTGGCTGACCATGTTTGATGCTGTGCTCATCCTCCTGCTCATCCCTC | |
| | TGAAGGACAAACTGGTCGATCCCATTTTGAGAAGACATGGCCTGCTCCCATCCTCCCT | |
| | GAAGAGGATCGCCGTGGGCATGTTCTTTGTCATGTGCTCAGCCTTTGCTGCAGGAATT | |
| | TTGGAGAGTAAAAGGCTGAACCTTGTTAAAGAGAAAACCATTAATCAGACCATCGGCA | |
| | ACGTCGTCTACCATGCTGCCGATCTGTCGCTGTGGTGGCAGGTGCCGCAGTACTTGCT | |
| | GATTGGGATCAGCGAGATCTTTGCAAGTATCGCAGGCCTGGAATTTGCATACTCAGCT | |
| | GCCCCCAAGTCCATGCAGAGTGCCATAATGGGCTTGTTCTTTTTCTTCTCTGGCGTCG | |
| | GGTCGTTCGTGGGTTCTGGACTGCTGGCACTGGTGTCTATCAAAGCCATCGGATGGAT | |
| | GAGCAGTCACACAGACTTTGGTAATATTAACGGCTGCTATTTGAACTATTACTTTTTT | |
| | CTTCTGGCTGCTATTCAAGGAGCTACCCTCCTGCTTTTCCTCATTATTTCTGTGAAAT | |
| | ATGACCATCATCGAGACCATCAGCGATCAAGAGCCAATGGCGTGCCCACCAGCAGGAG | |
| | GGCCTGACCTTCCTGAGGCCATGTGCGGTTTCTGAGGCTGACATGTCAGTAACTGACT | |
| | GGGGTGCACTGAGAACAGGCAAGACTTTAAATTCCCATAAAATGTCTGACTTCACTGA | |
| | AACTTGCATGTTGCCTGGATTGATTTCTTCTTTCCCTCTATCCAAAGGAGCTTGGTAA | |
| | GTGCCTTACTGCAGCGTGTCTCCTGGCACGCTGGGCCCTCCGGGAGGAGAGCTGCAGA | |
| | TTTCGAGTATGTCGCTTGTCATTCAAGGTCTCTGTGAATCCTCTAGCTGGGTTCCCTT | |
| | TTTTACAGAAACTCACAAATGGAGATTGCAAAGTCTTGGGGAACTCCACGTGTTAGTT | |
| | GGCATCCCAGTTTCTTAAACAAATAGTATCACCTGCTTCCCATAGCCATATCTCACTG | |
| | TAAAAAAAAAAATTAATAAACTGTTACTTATATTTAAGAAAGTGAGGATTTTTTTTTT | |
| | TTAAAGATAAAAGCATGGTCAGATGCTGCAAGGATTTTACATAAATGCCATATTTATG | |
| | GTTTCCTTCCTGAGAACAATCTTGCTCTTGCCATGTTCTTTGATTTAGGCTGGTAGTA | |
| | AACACATTTCATCTGCTGCTTCAAAAAGTACTTACTTTTTAAACCATCAACATTACTT | |
| | TTCTTTCTTAAGGCAAGGCATGCATAAGAGTCATTTGAGACCATGTGTCCCATCTCAA | |
| | GCCACAGAGCAACTCACGGGGTACTTCACACCTTACCTAGTCAGAGTGCTTATATATA | |
| | GCTTTATTTTGGTACGATTGAGACTAAAGACTGATCATGGTTGTATGTAAGGAAAACA | |
| | TTCTTTTGAACAGAAATAGTGTAATTAAAAATAATTGAAAGTGTTAAATGTGAACTTG | |
| | AGCTGTTTGACCAGTCACATTTTTGTATTGTTACTGTACGTGTATCTGGGGCTTCTCC | |
| | GTTTGTTAATACTTTTTCTGTATTTGTTGCTGTATTTTTGGCATAACTTTATTATAAA | |
| | AAGCATCTCAAATGCGAAAAAAAAAAAAAAAAAAAAAAA | |
| | ORF Start: ATG at 14 | ORF Stop: TGA at 1745 |
| | SEQ ID NO:42 | 577 aa MW at 62004.6 kD |

| | |
|---|---|
| NOV16a, | MEGSGGGAGERAPLLGARRAAAAAAAAGAFAGRRAACGAVLLTELLERAAFYGITSNL |
| CG103459-01 | VLFLNGAPFCWEGAQASEALLLFMGLTYLGSPFGGWLADARLGRARAILLSLALYLLG |
| Protein Sequence | MLAFPLLAAPATRAALCGSARLLNCTAPGPDAAARCCSPATFAGLVLVGLGVATVKAN |
| | ITPFGADQVKDRGPEATRRFFNWFYWSINLGAILSLGGIAYIQQNVSFVTGYAIPTVC |
| | VGLAFVAFLCGQSVFITKPPDGSAFTDMFKILTYSCCSQKRSGERQSNGEGIGVFQQS |
| | SKQSLFDSCKMSHGGPFTEEKVEDVKALVKIVPVFLALIPYWTVYFQMQTTYVLQSLH |
| | LRIPEISNITTTPHTLPAAWLTMFDAVLILLLIPLKDKLVDPILRRHGLLPSSLKRIA |
| | VGMFFVMCSAFAAGILESKRLNLVKEKTINQTIGNVVYHAADLSLWWQVPQYLLIGIS |
| | EIFASIAGLEFAYSAAPKSMQSAIMGLFFFFSGVGSFVGSGLLALVSIKAIGWMSSHT |
| | DFGNINGCYLNYYFFLLAAIQGATLLLFLIISVKYDHHRDHQRSRANGVPTSRRA |

Further analysis of the NOV16a protein yielded the following properties shown in Table 16B.

TABLE 16B
Protein Sequence Properties NOV16a

| | |
|---|---|
| PSort | 0.6000 probability located in plasma membrane; |
| analysis: | 0.4000 probability located in |
| | Golgi body; 0.3000 probability located |
| | in endoplasmic reticulum (membrane); |
| | 0.3000 probability located in microbody |
| | (peroxisome) |
| SignalP | No Known Signal Sequence Predicted |
| analysis: | |

A search of the NOV16a protein against the Geneseq database, a proprietary database that contains sequences published in patents and patent publication, yielded several homologous proteins shown in Table 16C.

TABLE 16C
Geneseq Results for NOV16a

| | | NOV16a | Identities/ | |
|---|---|---|---|---|
| | | Residues/ | Similarities for | |
| Geneseq | Protein/Organism/Length [Patent | Match | the Matched | Expect |
| Identifier | #, Date] | Residues | Region | Value |
| AAU12071 | Human PHT1 variant protein from | 1 . . . 577 | 577/577 (100%) | 0.0 |
| | Caco-2 cells - Homo sapiens, 577 | 1 . . . 577 | 577/577 (100%) | |
| | aa. [WO200192468-A2, 06-DEC-2001] | | | |
| AAU12068 | Human PHT1 protein isolated from | 1 . . . 577 | 577/577 (100%) | 0.0 |
| | Caco-2 cells - Homo sapiens, 577 | 1 . . . 577 | 577/577 (100%) | |
| | aa. [WO200192468-A2, 06-DEC-2001] | | | |
| AAU12070 | Human PHT1 variant protein from | 1 . . . 577 | 575/577 (99%) | 0.0 |
| | BeWo cells - Homo sapiens, 577 | 1 . . . 577 | 576/577 (99%) | |
| | aa. [WO200192468-A2, 06-DEC-2001] | | | |
| AAE16771 | Human transporter and ion channel-8 | 1 . . . 577 | 576/577 (99%) | 0.0 |
| | (TRICH-8) protein - Homo | 1 . . . 576 | 576/577 (99%) | |
| | sapiens, 576 aa. [WO200192304- | | | |
| | A2, 06-DEC-2001] | | | |
| AAB82821 | Human proton/oligonucleotide | 22 . . . 577 | 555/556 (99%) | 0.0 |
| | transporter hPHT1 polypeptide - | 1 . . . 556 | 555/556 (99%) | |
| | Homo sapiens, 556 aa. | | | |
| | [WO200160854-A1, 23-AUG-2001] | | | |

In a BLAST search of public sequence databases, the NOV16a protein was found to have homology to the proteins shown in the BLASTP data in Table 16D.

TABLE 16D
Public BLASTP Results for NOV16a

| | | NOV16a | Identities/ | |
|---|---|---|---|---|
| Protein | | Residues/ | Similarities for | |
| Accession | | Match | the Matched | Expect |
| Number | Protein/Organism/Length | Residues | Portion | Value |
| O09014 | PEPTIDE/HISTIDINE | 9 . . . 576 | 500/578 (86%) | 0.0 |
| | TRANSPORTER - Rattus | 3 . . . 571 | 531/578 (91%) | |
| | norvegicus (Rat), 572 aa. | | | |
| Q91W98 | SIMILAR TO PEPTIDE | 9 . . . 576 | 496/578 (85%) | 0.0 |
| | TRANSPORTER 3 - Mus | 3 . . . 573 | 531/578 (91%) | |
| | musculus (Mouse), 574 aa. | | | |
| AAH28394 | SIMILAR TO PEPTIDE | 117 . . . 577 | 460/461 (99%) | 0.0 |
| | TRANSPORTER 3 - Homo | 1 . . . 461 | 460/461 (99%) | |
| | sapiens (Human), 461 aa. | | | |
| Q9P2X9 | PEPTIDE TRANSPORTER 3 - | 9 . . . 558 | 289/570 (50%) | e−152 |
| | Homo sapiens (Human), 581 aa. | 14 . . . 564 | 379/570 (65%) | |
| Q9WU80 | CAMP INDUCIBLE 1 | 8 . . . 567 | 279/577 (48%) | e−144 |
| | PROTEIN - Mus musculus | 6 . . . 570 | 366/577 (63%) | |
| | (Mouse), 578 aa. | | | |

PFam analysis predicts that the NOV16a protein contains the domains shown in the Table 16E.

TABLE 16E
Domain Analysis of NOV16a

| | | Identities/ | |
|---|---|---|---|
| | | Similarities | |
| | NOV16a | for the | |
| Pfam Domain | Match Region | Matched Region | Expect Value |
| PTR2 | 103 . . . 496 | 109/448 (24%) | 6.7e−103 |
| | | 310/448 (69%) | |

### Example 17

The NOV17 clone was analyzed, and the nucleotide and encoded polypeptide sequences are shown in Table 17A.

TABLE 17A
NOV17 Sequence Analysis

| | SEQ ID NO:43 | 1393 bp |
|---|---|---|
| NOV17a, | CCCATGGAGGCTCCGGGACCCCGCGCCTTGCGGACTGCGCTCTGTGGCGGCTGTTGCT | |
| CG104210-01 | GCCTCCTCCTATGTGCCCAGCTGGCTGTGGCTGGTAAAGGAGCTCGAGGCTTTGGGAG | |
| DNA Sequence | GGGAGCCCTGATCCGCCTGAATATCTGGCCGGCGGTCCAAGGGGCCTGCAAACAGCTG | |
| | GAGGTCTGTGAGCACTGCGTGGAGGGAGACAGAGCGCGCAATCTCTCCAGCTGCATGT | |
| | GGGAGCAGTGCCGGCCAGAGGAGCCAGGTCACTGTGTGGCCCAATCTGAGGTGGTCAA | |
| | GGAAGGTTGCTCCATCTACAACCGCTCAGAGGCATGTCCAGCTGCTCACCACCACCCC | |
| | ACCTATGAACCGAAGACAGTCACAACAGGTAGCCCCCCAGTCCCTGAGGCCCACAGCC | |
| | CTGGATTTGACGGGGCCAGCTTTATCGGAGGTGTCGTGCTGGTGTTGAGCCTACAGGC | |
| | GGTGGCTTTCTTTGTGCTGCACTTCCTCAAGGCCAAGGACAGCACCTACCAGACGCTG | |
| | TGAGTACCTGGCCAGCAGCAAGTACCTGAGTCCCAGCTCACCTCCTGGTTCCTGCCCC | |
| | ACCGTTCCCCTTCAGTACCCAGGGTGCTGTCTTCTCCACTGGCAAGCCCTCAGGACGG | |
| | TGACAGCGTGCTCCATGTGAGCCACACCCCTTTTGTCTCCTCCAGTTGGGGTGTTTCC | |
| | TTTGTCAGATGTTGGCTGGGACCAGGACTCAGCCTGGGCCAGTCTAGGAGCCCAGCTG | |
| | AGCCCTCCTGTGTCTTTTCCCTTCATGCTGCCAGCAGGGAAGAGAACCAGTAGGTGCC | |
| | AGCCCAGCAACCTGTGGCCCGCGTTTCTGTGGCTGTGGGCAGGAGCTGGGCCTTGTGT | |
| | CTAGTTGGGTTTTGCTCTGAGAAGGGGAGCTGTGCTGAGGCCCTCTGTGTGCCGTGTG | |
| | TGCTGTGGGGCGGGTCGCCACAGCCTGTGTTAAAGTGTTTGCTCTTCCTCTGCTGCCT | |
| | CCTCTCGAGGCAGGGGGTCCTTGGCTGGCTGAGGCAGTGTCACCTTCCTGAGTGTCCT | |
| | CTTTGGCCTCTGCAGAATCTGACCCCTTTGGGCCTGGACTCCATCCTGAGGGGAAAGG | |
| | AGGATGCAGAGGGTGGCCTCTGGGCACCCTTGTGGGTAAGCGGGGGGCGGGGGCGGGA | |
| | AAAACTCTGGCCGCCAGTTTTTGGCTCCTGCGGGCACCAAGCAGGCTCAGTGTCTGAT | |
| | GCTTGACATCTCCTCCTGTCCTGGGCCTGGAACCTGCAGCTGAGAAAATCCCTCAACC | |
| | ACCTCGTCTCCTCCATCGCCCCTGCTGGGCCCCCCAGCCTGACAGTGGGTTGTATGCC | |
| | TGCCTCTTTCCACCAACTGGCCTGGGCACTGCCCCCAAATAAAGGAACTCTGCACTGC | |
| | A | |
| | ORF Start: ATG at 4 | ORF Stop: TGA at 523 |
| | SEQ ID NO:44 | 173 aa MW at 18421.0 kD |
| NOV17a, | MEAPGPRALRTALCGGCCCLLLCAQLAVAGKGARGFGRGALIRLNIWPAVQGACKQLE | |
| CG104210-01 | VCEHCVEGDRARNLSSCMWEQCRPEEPGHCVAQSEVVKEGCSIYNRSEACPAAHHHPT | |
| Protein Sequence | YEPKTVTTGSPPVPEAHSPGFDGASFIGGVVLVLSLQAVAFFVLHFLKAKDSTYQTL | |
| | SEQ ID NO:45 | 561 bp |
| NOV17b, | CCCATGGAGGCTCCGGGACCCCGCGCCTTGCGGACTGCGCTCTGTGGCGGCTGTTGCT | |
| CG104210-02 | GCCTCCTCCTATGTGCCCAGCTGGCTGTGGCTGGTAAAGGAGCTCGAGGCTTTGGGAG | |
| DNA Sequence | GGGAGCCCTGATCCGCCTGAATATCTGGCCGGCGGTCCAAGGGGCCTGCAAACAGCTG | |
| | GAGGTCTGTGAGCACTGCGTGGAGGGAGACAGAGCGCGCAATCTCTCCAGCTGCGTGT | |
| | GGGAGCAGTGCCGGCCAGAGGAGCCAGGACACTGTGTGGCCCAATCTGAGGTGGTCAA | |
| | GGAAGGTTGCTCCATCTACAACCGCTCAGAGGCATGTCCAGCTGCTCACCACCACCCC | |
| | ACCTATGAACCGAAGACAGTCACAACAGGGAGCCCCCCAGTCCCTGAGGCCCACAGCC | |
| | CTGGATTTGACGGGGCCAGCTTTATCGGAGGTGTCGTGCTGGTGTTGAGCCTACAGGC | |
| | GGTGGCTTTCTTTGTGCTGCACTTCCTCAAGGCCAAGGACAGCACCTACCAGACGCTG | |
| | TGAGTACCTGGCCAGCAGCAAGTACCTGAGTCCCAGCTC | |
| | ORF Start: ATG at 4 | ORF Stop: TGA at 523 |
| | SEQ ID NO:46 | 173 aa MW at 18389.0 kD |
| NOV17b, | MEAPGPRALRTALCGGCCCLLLCAQLAVAGKGARGFGRGALIRLNIWPAVQGACKQLE | |
| CG104210-02 | VCEHCVEGDRARNLSSCVWEQCRPEEPGHCVAQSEVVKEGCSIYNRSEACPAAHHHPT | |
| Protein Sequence | YEPKTVTTGSPPVPEAHSPGFDGASFIGGVVLVLSLQAVAFFVLHFLKAKDSTYQTL | |
| | SEQ ID NO:47 | 349 bp |
| NOV17c, | CACCGGATCCGGTAAAGGAGCTCGAGGCTTTGGGAGGGGAGCCCTGATCCGCCTGAAT | |
| 272249075 DNA | ATCTGGCCGGCGGTCCAAGGGGCCTGCAAACAGCTGGAGGTCTGTGAGCACTGCGTGG | |
| Sequence | AGGGAGACAGAGCGCGCAATCTCTCCAGCTGCATGTGGGAGCAGTGCCGGCCAGAGGA | |
| | GCCAGGACACTGTGTGGCCCAATCTGAGGTGGTCAAGGAAGGTTGCTCCATCTACAAC | |
| | CGCTCAGAGGCATGTCCAGCTGCTCACCACCACCCCACCTATGAACCGAAGACAGTCA | |
| | CAACAGGGAGCCCCCCAGTCCCTGAGGCCCACAGCCCTGGATTTGACGGGGTCGACGG | |
| | C | |
| | ORF Start: at 2 | ORF Stop: end of sequence |
| | SEQ ID NO:48 | 116 aa MW at 12383.7 kD |

| | |
|---|---|
| NOV17c, | TGSGKGARGFGRGALIRLNIWPAVQGACKQLEVCEHCVEGDRARNLSSCMWEQCRPEE |
| 272249075 | PGHCVAQSEVVKEGCSIYNRSEACPAAHHHPTYEPKTVTTGSPPVPEAHSPGFDGVDG |
| Protein Sequence | |

Sequence comparison of the above protein sequences yields the following sequence relationships shown in Table 17B.

TABLE 17B
Comparison of NOV17a against NOV17b and NOV17c.

| | NOV17a Residues/ | Identities/Similarities |
|---|---|---|
| Protein Sequence | Match Residues | for the Matched Region |
| NOV17b | 1 . . . 173 | 139/173 (80%) |
| | 1 . . . 173 | 140/173 (80%) |
| NOV17c | 41 . . . 139 | 99/99 (100%) |
| | 15 . . . 113 | 99/99 (100%) |

Further analysis of the NOV17a protein yielded the following properties shown in Table 17C.

TABLE 17C
Protein Sequence Properties NOV17a

| | |
|---|---|
| PSort | 0.6850 probability located in endoplasmic |
| analysis: | reticulum (membrane); 0.6400 |
| | probability located in plasma membrane; |
| | 0.4600 probability located in Golgi |
| | body; 0.1000 probability located in |
| | endoplasmic reticulum (lumen) |
| SignalP | Cleavage site between residues 30 and 31 |
| analysis: | |

A search of the NOV17a protein against the Geneseq database, a proprietary database that contains sequences published in patents and patent publication, yielded several homologous proteins shown in Table 17D.

TABLE 17D
Geneseq Results for NOV17a

| | | NOV17a | Identities/ | |
|---|---|---|---|---|
| | | Residues/ | Similarities for | |
| Geneseq | Protein/Organism/Length [Patent | Match | the Matched | Expect |
| Identifier | #, Date] | Residues | Region | Value |
| AAE03827 | Human gene 10 encoded secreted | 1 . . . 173 | 173/173 (100%) | e−103 |
| | protein HBINS58, SEQ ID NO: 73 - | 1 . . . 173 | 173/173 (100%) | |
| | Homo sapiens, 173 aa. | | | |
| | [WO200136440-A1, 25-MAY-2001] | | | |
| AAE03852 | Human gene 10 encoded secreted | 1 . . . 160 | 159/160 (99%) | 5e−94 |
| | protein HBINS58, SEQ ID NO: 98 - | 1 . . . 160 | 159/160 (99%) | |
| | Homo sapiens, 210 aa. | | | |
| | [WO200136440-A1, 25-MAY-2001] | | | |
| AAB58415 | Lung cancer associated polypeptide | 73 . . . 173 | 41/124 (33%) | 8e−10 |
| | sequence SEQ ID 753 - Homo | 95 . . . 214 | 56/124 (45%) | |
| | sapiens, 214 aa. [WO200055180- | | | |
| | A2, 21-SEP-2000] | | | |
| AAG03771 | Human secreted protein, SEQ ID | 73 . . . 173 | 38/124 (30%) | 1e−07 |
| | NO: 7852 - Homo sapiens, 197 aa. | 78 . . . 197 | 52/124 (41%) | |
| | [EP1033401-A2, 06-SEP-2000] | | | |
| ABB65987 | Drosophila melanogaster | 116 . . . 173 | 29/60 (48%) | 9e−05 |
| | polypeptide SEQ ID NO: 24753 - | 127 . . . 183 | 35/60 (58%) | |
| | Drosophila melanogaster, 183 aa. | | | |
| | [WO200171042-A2, 27-SEP-2001] | | | |

In a BLAST search of public sequence databases, the NOV17a protein was found to have homology to the proteins shown in the BLASTP data in Table 17E.

TABLE 17E
Public BLASTP Results for NOV17a

| | | NOV17a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9D6W7 | 2310047N01RIK PROTEIN - | 1 . . . 173 | 140/173 (80%) | 1e−82 |
| | Mus musculus (Mouse), 172 aa. | 1 . . . 171 | 150/173 (85%) | |
| Q9BPV0 | CD164 ISOFORM DELTA 4 - | 73 . . . 173 | 41/111 (36%) | 6e−11 |
| | Homo sapiens (Human), 184 aa. | 78 . . . 184 | 56/111 (49%) | |
| Q9CVT7 | CD164 ANTIGEN - Mus | 25 . . . 173 | 51/173 (29%) | 2e−10 |
| | musculus (Mouse), 161 aa | 5 . . . 161 | 67/173 (38%) | |
| | (fragment). | | | |
| Q9QX82 | ENDOLYN PRECURSOR - | 54 . . . 173 | 41/140 (29%) | 4e−10 |
| | Rattus norvegicus (Rat), 195 aa. | 57 . . . 195 | 59/140 (41%) | |
| Q9Z317 | MGC-24V - Mus musculus | 54 . . . 173 | 44/144 (30%) | 7e−10 |
| | (Mouse), 197 aa. | 58 . . . 197 | 58/144 (39%) | |

### Example 18

The NOV18 clone was analyzed, and the nucleotide and encoded polypeptide sequences are shown in Table 18A.

TABLE 18A
NOV18 Sequence Analysis

| | SEQ ID NO:49 | 788 bp |
|---|---|---|
| NOV18a, | CTTTTGCCTTTATGCAACCAACATGGAGATTTTGTACCATGTCCTGTTCTTAGTGCTT | |
| CG104251-01 | GAATGTCCTAACCTGAAGCTGAAGAAGCCGCCCTGGCTGCACATGCTGTCGGCCATGA | |
| DNA Sequence | CTGTATGCTCTGGTGGTGGTGTCTTCCTCATTACCGGAGGAATCATTTATGATGTTAT | |
| | TGTTGAACCTCCAAGTGTTGGCTCTATGACTGATGAACATGGGCATCAGAGGCCAGTA | |
| | GCTTTCTTTGCCTATAGAGTAAATGGACAATATATTATGGAAGGACTTGCATCCAGCT | |
| | TCCTGTTTACAATGGGAGGTTTAGGTTTCATAATCCTGGACCAATTGAATGCACCAAA | |
| | TATCCCAAAACTCAATAGATTTCTTCTTCTATTCATTGGATTTGTCTGTGTTCTATTG | |
| | AGTATTTTCATGGCTAGAGTATTCATGAGAATGAAACTGCCGAGCTATCTGATGGGTT | |
| | AGAGTGCCTTTGAGAAGAAATCAGTGGATACTGGATTTTTTCTTGTCAATGAAGTTTT | |
| | AAAGGCTGTACCAATCCTCTAATATGAAATGTGGAAAAGAATGAAGAGCAGCAGTAAA | |
| | AGAAATATCTAGTGAAAAAACAGGAAGCGTATTGAAGCTTGGACTAGAATTTCTTCTT | |
| | GGTATTAAAGAGACAAGTTTATCACAGAATTTTTTTTCCTGCTGGCCTATTGCTATAC | |
| | CAATGATGTTGAGTGGCATTTTCTTTTTAGTTTTTCATTAAAATATATTCCATATCTA | |
| | CAACTATAATATCAAATAAAGTGATTATTTTTTA | |
| | ORF Start: ATG at 23 | ORF Stop: TAG at 464 |
| | SEQ ID NO:50 | 147 aa MW at 16447.7 kD |

| | |
|---|---|
| NOV18a, | MEILYHVLFLVLECPNLKLKKPPWLHMLSAMTVCSGGGVFLITGGIIYDVIVEPPSVG |
| CG104251-01 | SMTDEHGHQRPVAFFAYRVNGQYIMEGLASSFLFTMGGLGFIILDQLNAPNIPKLNRF |
| Protein Sequence | LLLFIGFVCVLLSIFMARVFMRMKLPSYLMG |

Further analysis of the NOV18a protein yielded the following properties shown in Table 18B.

TABLE 18B
Protein Sequence Properties NOV18a

| | |
|---|---|
| PSort | 0.6400 probability located in plasma membrane; |
| analysis: | 0.4600 probability located in |
| | Golgi body; 0.3700 probability located in |
| | endoplasmic reticulum (membrane); |
| | 0.1000 probability located in endoplasmic |
| | reticulum (lumen) |
| SignalP | Cleavage site between residues 42 and 43 |
| analysis: | |

A search of the NOV18a protein against the Geneseq database, a proprietary database that contains sequences published in patents and patent publication, yielded several homologous proteins shown in Table 18C.

TABLE 18C
Geneseq Results for NOV18a

| | | NOV18a | Identities/ | |
|---|---|---|---|---|
| | | Residues/ | Similarities for | |
| Geneseq | Protein/Organism/Length [Patent | Match | the Matched | Expect |
| Identifier | #, Date] | Residues | Region | Value |
| AAY53631 | A bone marrow secreted protein | 1 . . . 147 | 133/149 (89%) | 1e−69 |
| | designated BMS155 - Homo | 1 . . . 149 | 135/149 (90%) | |
| | sapiens, 149 aa. [WO9933979-A2, | | | |
| | 08-JUL-1999] | | | |
| AAY53042 | Human secreted protein clone | 1 . . . 147 | 133/149 (89%) | 1e−69 |
| | pu282_10 protein sequence SEQ ID | 1 . . . 149 | 135/149 (90%) | |
| | NO: 90 - Homo sapiens, 149 aa. | | | |
| | [WO9957132-A1, 11-NOV-1999] | | | |
| AAB12143 | Hydrophobic domain protein | 1 . . . 147 | 133/149 (89%) | 1e−69 |
| | isolated from WERI-RB cells - | 1 . . . 149 | 135/149 (90%) | |
| | Homo sapiens, 149 aa. | | | |
| | [WO200029448-A2, 25-MAY-2000] | | | |
| AAY59670 | Secreted protein 108-005-5-0-F6-FL - | 1 . . . 147 | 133/149 (89%) | 1e−69 |
| | Homo sapiens, 149 aa. | 1 . . . 149 | 135/149 (90%) | |
| | [WO9940189-A2, 12-AUG-1999] | | | |
| AAY60146 | Human endometrium tumor EST | 1 . . . 147 | 133/149 (89%) | 1e−69 |
| | encoded protein 206 - Homo | 23 . . . 171 | 135/149 (90%) | |
| | sapiens, 171 aa. [DE19817948-A1, | | | |
| | 21-OCT-1999] | | | |

In a BLAST search of public sequence databases, the NOV18a protein was found to have homology to the proteins shown in the BLASTP data in Table 18D.

TABLE 18D
Public BLASTP Results for NOV18a

| | | NOV18a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9NRP0 | DC2 (HYDROPHOBIC PROTEIN | 1 . . . 147 | 133/149 (89%) | 3e−69 |
| | HSF-28) (HYPOTHETICAL 16.8 | 1 . . . 149 | 135/149 (90%) | |
| | KDA PROTEIN) - Homo sapiens | | | |
| | (Human), 149 aa. | | | |
| Q9P075 | HSPC307 - Homo sapiens | 1 . . . 147 | 133/149 (89%) | 3e−69 |
| | (Human), 167 aa (fragment). | 19 . . . 167 | 135/149 (90%) | |
| Q9CPZ2 | 2310008M10RIK PROTEIN | 1 . . . 147 | 132/149 (88%) | 6e−69 |
| | (RIKEN CDNA 2310008M10 | 1 . . . 149 | 135/149 (90%) | |
| | GENE) - Mus musculus (Mouse), | | | |
| | 149 aa. | | | |
| Q9P1R4 | HDCMD45P - Homo sapiens | 1 . . . 147 | 132/149 (88%) | 2e−68 |
| | (Human), 160 aa (fragment). | 12 . . . 160 | 134/149 (89%) | |
| AAH24224 | SIMILAR TO DC2 PROTEIN - | 40 . . . 147 | 96/108 (88%) | 7e−50 |
| | Homo sapiens (Human), 119 aa. | 12 . . . 119 | 100/108 (91%) | |

### Example 19

The NOV19 clone was analyzed, and the nucleotide and encoded polypeptide sequences are shown in Table 19A.

TABLE 19A
NOV19 Sequence Analysis

| | SEQ ID NO:51 | 3761 bp |
|---|---|---|
| NOV19a, | GGGCGCGCCGAGCCGGGCGCGGGGGCGCTGAACGGCGGAGCGGGAGCGGCCGGAGGAG | |
| CG104934-01 | CCATGGACTGCAGCCTCGTGCGGACGCTCGTGCACAGATACTGTGCAGGAGAAGAGAA | |
| DNA Sequence | TTGGGTGGACAGCAGGACCATCTACGTGGGACACAGGGAGCCACCTCCGGGCGCAGAG | |
| | GCCTACATCCCACAGAGATACCCAGACAACAGGATCGTCTCGTCCAAGTACACATTTT | |
| | GGAACTTTATACCCAAGAATTTATTTGAACAATTCAGAAGAGTAGCCAACTTTTATTT | |
| | CCTTATCATATTTCTGGTGCAGTTGATTATTGATACACCCACAAGTCCAGTGACAAGC | |
| | GGACTTCCACTCTTCTTTGTCATTACTGTGACGGCTATCAAACAGGGTTATGAAGACT | |
| | GGCTTCGACATAAAGCAGACAATGCCATGAACCAGTGTCCTGTTCATTTCATTCAGCA | |
| | CGGCAAGCTCGTTCGGAAACAAAGTCGAAAGCTGCGAGTTGGGGACATTGTCATGGTT | |
| | AAGGAGGACGAGACCTTTCCCTGCGACTTGATCTTCCTTTCCAGCAACCGGGGAGATG | |
| | GGACGTGCCACGTCACCACCGCCAGCTTGGATGGAGAATCCAGCCATAAAACGCATTA | |
| | CGCGGTCCAGGACACCAAAGGCTTCCACACAGAGGAGGATATCGGCGGACTTCACGCC | |
| | ACCATCGAGTGTGAGCAGCCCCAGCCCGACCTCTACAAGTTCGTGGGTCGCATCAACG | |
| | TTTACAGTGACCTGAATGACCCCGTGGTGAGGCCCTTAGGATCGGAAAACCTGCTGCT | |
| | TAGAGGAGCTACACTGAAGAACACTGAGAAAATCTTTGGTGTGGCTATTTACACGGGA | |
| | ATGGAAACCAAGATGGCATTAAATTATCAATCAAAATCTCAGAAGCGATCTGCCGTGG | |
| | AAAAATCGATGAATGCGTTCCTCATTGTGTATCTCTGCATTCTGATCAGCAAAGCCCT | |
| | GATAAACACTGTGCTGAAATACATGTGGCAGAGTGAGCCCTTTCGGGATGAGCCGTGG | |
| | TATAATCAGAAAACGGAGTCGGAAAGGCAGAGGAATCTGTTCCTCAAGGCATTCACGG | |
| | ACTTCCTGGCCTTCATGGTCCTCTTTAACTACATCATCCCTGTGTCCATGTACGTCAC | |
| | GGTCGAGATGCAGAAGTTCCTCGGCTCTTACTTCATCACCTGGGACGAAGACATGTTT | |
| | GACGAGGAGACTGGCGAGGGGCCTCTGGTGAACACGTCGGACCTCAATGAAGAGCTGG | |
| | GACAGGTGGAGTACATCTTCACAGACAAGACCGGCACCCTCACGGAAAACAACATGGA | |
| | GTTCAAGGAGTGCTGCATCGAAGGCCATGTCTACGTGCCCCACGTCATCTGCAACGGG | |
| | CAGGTCCTCCCAGAGTCGTCAGGAATCGACATGATTGACTCGTCCCCCAGCGTCAACG | |
| | GGAGGGAGCGCGAGGAGCTGTTTTTCCGGGCCCTCTGTCTCTGCCACACCGTCCAGGT | |
| | GAAAGACGATGACAGCGTAGACGGCCCCAGGAAATCGCCGGACGGGGGGAAATCCTGT | |
| | GTGTACATCTCATCCTCGCCCGACGAGGTGGCGCTGGTCGAAGGTGTCCAGAGACTTG | |
| | GCTTTACCTACCTAAGGCTGAAGGACAATTACATGGAGATATTAAACAGGGAGAACCA | |
| | CATCGAAAGGTTTGAATTGCTGGAAATTTTGAGTTTTGACTCAGTCAGAAGGAGAATG | |
| | AGTGTAATTGTAAAATCTGCTACAGGAGAAATTTATCTGTTTTGCAAAGGAGCAGATT | |
| | CTTCGATATTCCCCCGAGTGATAGAAGGCAAAGTTGACCAGATCCGAGCCAGAGTGGA | |
| | GCGTAACGCAGTGGAGGGGCTCCGAACTTTGTGTGTTGCTTATAAAAGGCTGATCCAA | |
| | GAAGAATATGAAGGCATTTGTAAGCTGCTGCAGGCTGCCAAAGTGGCCCTTCAAGATC | |
| | GAGAGAAAAAGTTAGCAGAAGCCTATGAGCAAATAGAGAAAGATCTTACTCTGCTTGG | |
| | TGCTACAGCTGTTGAGGACCGGCTGCAGGAGAAAGCTGCAGACACCATCGAGGCCCTG | |
| | CAGAAGGCCGGGATCAAAGTCTGGGTTCTCACGGGAGACAAGATGGAGACGGCCGCGG | |
| | CCACGTGCTACGCCTGCAAGCTCTTCCGCAGGAACACGCAGCTGCTGGAGCTGACCAC | |
| | CAAGAGGATCGAGGAGCAGAGCCTGCACGACGTCCTGTTCGAGCTGAGCAAGACGGTC | |
| | CTGCGCCACAGCGGGAGCCTGACCAGAGACAACCTGTCCGGACTTTCAGCAGATATGC | |
| | AGGACTACGGTTTAATTATCGACGGAGCTGCACTGTCTCTGATAATGAAGCCTCGAGA | |
| | AGACGGGAGTTCCGGCAACTACAGGGAGCTCTTCCTGGAAATCTGCCGGAGCTGCAGC | |
| | GCGGTGCTCTGCTGCCGCATGGCGCCCTTGCAGAAGGCTCAGATTGTTAAATTAATCA | |
| | AATTTTCAAAAGAGCACCCAATCACGTTAGCAATTGGCGATGGTGCAAATGATGTCAG | |
| | CATGATTCTGGAAGCGCACGTGGGCATAGGTGTCATCGGCAAGGAAGGCCGCCAGGCT | |
| | GCCAGGAACAGCGACTATGCAATCCCAAAGTTTAAGCATTTGAAGAAGATGCTGCTTG | |
| | TTCACGGGCATTTTTATTACATTAGGATCTCTGAGCTCGTGCAGTACTTCTTCTATAA | |
| | GAACGTCTGCTTCATCTTCCCTCAGTTTTTATACCAGTTCTTCTGTGGGTTTTCACAA | |
| | CAGACTTTGTACGACACCGCGTATCTGACCCTCTACAACATCAGCTTCACCTCCCTCC | |
| | CCATCCTCCTGTACAGCCTCATGGAGCAGCATGTTGGCATTGACGTGCTCAAGAGAGA | |
| | CCCGACCCTGTACAGGGACGTCGCCAAGAATGCCCTGCTGCGCTGGCGCGTGTTCATC | |
| | TACTGGACGCTCCTGGGACTGTTTGACGCACTGGTGTTCTTCTTTGGTGCTTATTTCG | |
| | TGTTTGAAAATACAACTGTGACAAGCAACGGGCAGATATTTGGAAACTGGACGTTTGG | |
| | AACGCTGGTATTCACCGTGATGGTGTTCACAGTTACACTAAAGCTTGCATTGGACACA | |
| | CACTACTGGACTTGGATCAACCATTTTGTCATCTGGGGGTCGCTGCTGTTCTACGTTG | |
| | TCTTTTCGCTTCTCTGGGGAGGAGTGATCTGGCCGTTCCTCAACTACCAGAGGATGTA | |
| | CTACGTGTTCATCCAGATGCTGTCCAGCGGGCCCGCCTGGCTGGCCATCGTGCTGCTG | |
| | GTGACCATCAGCCTCCTTCCCGACGTCCTCAAGAAAGTCCTGTGCCGGCAGCTGTGGC | |
| | CAACAGCAACAGAGAGAGTCCAGAATGGGTGCGCACAGCCTCGGGACCGCGACTCAGA | |
| | ATTCACCCCTCTTGCCTCTCTGCAGAGCCCAGGCTACCAGAGCACCTGTCCCTCGGCC | |
| | GCCTGGTACAGCTCCCACTCTCAGCAGGTGACACTCGCGGCCTGGAAGGAGAAGGTGT | |
| | CCACGGAGCCCCCACCCATCCTCGGCGGTTCCCATCACCACTGCAGTTCCATCCCAAG | |
| | TCACAGCTGCCCTAGGTCCCGTGTGGGAATGCTCGTGTGATGGATGGTCCTAAGCCTG | |
| | TGGAGACTGTGCACGTGCCTCTTCCTGGCCCCCAGCAGGCAAGGAGGGGGGTCACAGG | |
| | CCTTGCCCTCGAGCATGGCACCCTGGCCGCCTGGACCCAGCACTGTGGT | |
| | ORF Start: ATG at 61 | ORF Stop: TGA at 3634 |
| | SEQ ID NO:52 | 1191 aa MW at 135846.0 kD |

| | |
|---|---|
| NOV19a, | MDCSLVRTLVHRYCAGEENWVDSRTIYVGHREPPPGAEAYIPQRYPDNRIVSSKYTFW |
| CG104934-01 | NFIPKNLFEQFRRVANFYFLIIFLVQLIIDTPTSPVTSGLPLFFVITVTAIKQGYEDW |
| Protein Sequence | LRHKADNAMNQCPVHFIQHGKLVRKQSRKLRVGDIVMVKEDETFPCDLIFLSSNRGDG |
| | TCHVTTASLDGESSHKTHYAVQDTKGFHTEEDIGGLHATIECEQPQPDLYKFVGRINV |
| | YSDLNDPVVRPLGSENLLLRGATLKNTEKIFGVAIYTGMETKMALNYQSKSQKRSAVE |
| | KSMNAFLIVYLCILISKALINTVLKYMWQSEPFRDEPWYNQKTESERQRNLFLKAFTD |
| | FLAFMVLFNYIIPVSMYVTVEMQKFLGSYFITWDEDMFDEETGEGPLVNTSDLNEELG |
| | QVEYIFTDKTGTLTENNMEFKECCIEGHVYVPHVICNGQVLPESSGIDMIDSSPSVNG |
| | REREELFFRALCLCHTVQVKDDDSVDGPRKSPDGGKSCVYISSSPDEVALVEGVQRLG |
| | FTYLRLKDNYMEILNRENHIERFELLEILSFDSVRRRMSVIVKSATGEIYLFCKGADS |
| | SIFPRVIEGKVDQIRARVERNAVEGLRTLCVAYKRLIQEEYEGICKLLQAAKVALQDR |
| | EKKLAEAYEQIEKDLTLLGATAVEDRLQEKAADTIEALQKAGIKVWVLTGDKMEDAAA |
| | TCYACKLFRRNTQLLELTTKRIEEQSLHDVLFELSKTVLRHSGSLTRDNLSGLSADMQ |
| | DYGLIIDGAALSLIMKPREDGSSGNYRELFLEICRSCSAVLCCRMAPLQKAQIVKLIK |
| | FSKEHPITLAIGDGANDVSMILEAHVGIGVIGKEGRQAARNSDYAIPKFKHLKKMLLV |
| | HGHFYYIRISELVQYFFYKNVCFIFPQFLYQFFCGFSQQTLYDTAYLTLYNISFTSLP |
| | ILLYSLMEQHVGIDVLKRDPTLYRDVAKNALLRWRVFIYWTLLGLFDALVFFFGAYFV |
| | FENTTVTSNGQIFGNWTFGTLVFTVMVFTVTLKLALDTHYWTWINHFVIWGSLLFYVV |
| | FSLLWGGVIWPFLNYQRMYYVFIQMLSSGPAWLAIVLLVTISLLPDVLKKVLCRQLWP |
| | TATERVQNGCAQPRDRDSEFTPLASLQSPGYQSTCPSAAWYSSHSQQVTLAAWKEKVS |
| | TEPPPILGGSHHHCSSIPSHSCPRSRVGMLV |

Further analysis of the NOV19a protein yielded the following properties shown in Table 19B.

TABLE 19B
Protein Sequence Properties NOV19a

| | |
|---|---|
| PSort | 0.6000 probability located in plasma membrane; |
| analysis: | 0.4000 probability located in |
| | Golgi body; 0.3000 probability located |
| | in endoplasmic reticulum (membrane); |
| | 0.0300 probability located in mitochondrial |
| | inner membrane |
| SignalP | No Known Signal Sequence Predicted |
| analysis: | |

A search of the NOV19a protein against the Geneseq database, a proprietary database that contains sequences published in patents and patent publication, yielded several homologous proteins shown in Table 19C.

TABLE 19C
Geneseq Results for NOV19a

| | | NOV19a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAO14200 | Human transporter and ion | 1 . . . 1191 | 1190/1192 (99%) | 0.0 |
| | channel TRICH-17 - Homo | 1 . . . 1192 | 1191/1192 (99%) | |
| | sapiens, 1192 aa. [WO200204520- | | | |
| | A2, 17-JAN-2002] | | | |
| AAB42368 | Human ORFX ORF2132 | 338 . . . 1109 | 770/772 (99%) | 0.0 |
| | polypeptide sequence SEQ ID | 1 . . . 772 | 772/772 (99%) | |
| | NO: 4264 - Homo sapiens, 797 aa. | | | |
| | [WO200058473-A2, 05-OCT-2000] | | | |
| AAG67546 | Amino acid sequence of a human | 22 . . . 1109 | 657/1119 (58%) | 0.0 |
| | transporter protein - Homo | 18 . . . 1106 | 833/1119 (73%) | |
| | sapiens, 1177 aa. [WO200164878- | | | |
| | A2, 07-SEP-2001] | | | |
| AAO14203 | Human transporter and ion | 22 . . . 1109 | 583/1119 (52%) | 0.0 |
| | channel TRICH-20 - Homo | 18 . . . 1040 | 755/1119 (67%) | |
| | sapiens, 1096 aa. [WO200204520- | | | |
| | A2, 17-JAN-2002] | | | |
| AAM39290 | Human polypeptide SEQ ID NO: | 370 . . . 1109 | 424/771 (54%) | 0.0 |
| | 2435 - Homo sapiens, 815 aa. | 1 . . . 744 | 544/771 (69%) | |
| | [WO200153312-A1, 26-JUL-2001] | | | |

In a BLAST search of public sequence databases, the NOV19a protein was found to have homology to the proteins shown in the BLASTP data in Table 19D.

TABLE 19D
Public BLASTP Results for NOV19a

| | | NOV19a | Identities/ | |
|---|---|---|---|---|
| Protein | | Residues/ | Similarities for | |
| Accession | | Match | the Matched | Expect |
| Number | Protein/Organism/Length | Residues | Portion | Value |
| P98197 | Potential phospholipid- | 1 . . . 1189 | 1074/1195 (89%) | 0.0 |
| | transporting ATPase IH (EC | 1 . . . 1185 | 1117/1195 (92%) | |
| | 3.6.3.1) - Mus musculus (Mouse), | | | |
| | 1187 aa. | | | |
| P98196 | Potential phospholipid- | 338 . . . 1109 | 772/772 (100%) | 0.0 |
| | transporting ATPase IS (EC | 1 . . . 772 | 772/772 (100%) | |
| | 3.6.3.1) - Homo sapiens (Human), | | | |
| | 797 aa (fragment). | | | |
| Q8WX24 | BB206I21.1 (ATPASE, CLASS | 14 . . . 997 | 633/992 (63%) | 0.0 |
| | VI, TYPE 11C) - Homo sapiens | 1 . . . 962 | 770/992 (76%) | |
| | (Human), 962 aa (fragment). | | | |
| Q9N0Z4 | RING-FINGER BINDING | 22 . . . 1109 | 574/1123 (51%) | 0.0 |
| | PROTEIN - Oryctolagus | 10 . . . 1036 | 752/1123 (66%) | |
| | cuniculus (Rabbit), 1107 aa | | | |
| | (fragment). | | | |
| Q9Y2G3 | Potential phospholipid- | 486 . . . 1109 | 358/625 (57%) | 0.0 |
| | transporting ATPase IR (EC | 1 . . . 601 | 462/625 (73%) | |
| | 3.6.3.1) - Homo sapiens (Human), | | | |
| | 672 aa (fragment). | | | |

PFam analysis predicts that the NOV19a protein contain the domains shown in the Table 19E.

TABLE 19E
Domain Analysis of NOV19a

| | | Identities/ | |
|---|---|---|---|
| | | Similarities | |
| | NOV19a | for the | |
| Pfam Domain | Match Region | Matched Region | Expect Value |
| Hydrolase | 408 . . . 846 | 46/448 (10%) | 0.0058 |
| | | 258/448 (58%) | |

### Example 20

The NOV20 clone was analyzed, and the nucleotide and encoded polypeptide sequences are shown in Table 20A.

TABLE 20A
NOV20 Sequence Analysis

| | SEQ ID NO:53 | 2588 bp |
|---|---|---|
| NOV20a, | AGTTCCGAACAGAAGGCTGTGTATTCTCTGCCGCTTATTGTGGCCTCGACAGGCCATG | |
| CG105463-01 | GTTACTTTGGCCACTGCCAGAGCAGCCTTGGCACTATGGAGGAGCCTAGGGCTACCCC | |
| DNA Sequence | TCAGCTGTACTTGGGGCTGGTCCTGCAGTTGCTACCCAGGGTTATGGCAGCACTGCCT | |
| | GAAGGTGTGAGACCAAATTCGAATCCTTATGGTTTTCCATGGGAATTGGTGATATGTG | |
| | CAGCTGTCCTTGGATTTGTTGCTGTTCCCTTTTTTTTGTGGAGAAGTTTTAGATCGGT | |
| | TAGGAGTCGGCTTTATGTGGGAAGAGAGAAAGAGCTTGCTATAGCGCTTTCTGGACTA | |
| | ATTGAAGAAAAATGTAGACTACTTGAAAAATTTAGCCTTGTTCAAAAAGAGTATGAAG | |
| | GCTATGAAGTAGAGTCATCTTTAGAGGATGCCAGCTTTGAGAAGGAGGCAACAGAAGC | |
| | ACAAAGTCTGGAGGCAAACTGTGAAAAGCTGAACAGGTCCAATTCTGAACTGGAGCAT | |
| | GAAATACTCTGTCTAGAAAAGGGGATAAAAGAAGAGAAATCTAAACATTCTGAACAAG | |
| | ATGAGGTGATGGCAGATATTTCCAAAAAGATACAGTCTCTAGAAGATGAGTCAAAATC | |
| | CCTCAAATCACTACTAACTGAAGCCAAAATGACCTTCAAGGGATTTCAAATGAATGAA | |
| | GAAAAACTGGAGATAGGAATACAAGATGCTTCGAGTGAAAATTGTCAACTTCAGGAAA | |
| | GCCAGAAACAGCTTTTGCAAGAAGCTGAAGTATGGAAAGAACAAGTGAGTGAACTTAA | |
| | TAAACAGAAAATAACATTTGAAGACTCCAAAGTACACGCAGAACAAGTTCTAAATGAT | |
| | AAAGAAAATCACATCGAGACTCTGACTGAACGCTTGCTAAAGATCAAAGATCAGGCTG | |
| | CTGTGCTGGAAGAAGACATAACGGATGATGGTAACTTGGAATTAGAAATGAACAGTGA | |
| | ATTGAAAGATGGTGCTTACTTAGATAATCCTCCAAAAGGAGCTTTGAAGAAACTGATT | |
| | CATGCTGCTAAGTTAAATGCTTCTTTAACAACCTTAGAAGGAGAAAGAAACCAATTTA | |
| | TATTCAGTTATCTGAAGTTGATAAAACCAAGGAAGAGCTTAGAGAGCATATTAAAAAT | |
| | CTTCAGACGGAACAAGCATCTTTGCAGTCGGAAAACACACATTTTGAAAGTGAGAATC | |
| | AGAAACTTCAACAGAAAGTTAATGACTGAGTTATATCAAGAAAATGAAATGAAACTCT | |
| | ACAGGAAATTAATAGTAGAGGAAAATAACCGGTTAGAGAAAGAGAAACTTTCTAAAGT | |
| | AGACGAAATGATCAGCCATGCCACTGAAGAGCTGGAGACCTGCAGAAAGCGAGCCAAA | |
| | GATCTTGAAGAAGAACTTGAGAGAACTATTCTTTTTTATCAAGGGAAGATTATATACC | |
| | ATGAGAAAAAAGCACATGATAATTGTTTGGCAGCATGGACTGCTGAAAGAAACCTCAA | |
| | TGATTTAAGGAAAGAAAATGCTCACAAAAGACAAAAATTAGCTGAAACAGAGTTTAAA | |
| | ATTAAACTTTTAGAAAAAGATCCTTATGCACTTGATGTTCCAAATACAGCATTTGGCA | |
| | GAGAGCATTCCTCATATGGTCCCTCACCATTGGGTCGGCCTTCATCTGAAACGAGAGC | |
| | TTTTCTCTATCTTCCGACTTTGTTGGAGGGTCCACTGAGACTCTCACCTTTGCTTCCA | |
| | GGGGGAGGAGGAAGAGACCCAAGAGGCCCAGGGAATCCTCTGGACCACCAGATTACCA | |
| | AGGAAAGAGGAGAATCAAGCTGTGATAGGTTTACTGATCCTCACAAGGCTCCTTCTGA | |
| | CACTGGGCCCCTGTCACCTCCGTGGGAACAGGACCGTAGGATGATGTTTCCTCCACCA | |
| | GGACAATCATATCCTGATTCAGCTCTTCCTCCACAAAGGCAAGACAGATTTTATTCTA | |
| | ATTCTGCTAGACGCTCTGGACTAGCAGAACTCAGAAGTTTTAATATACCTTCTTTGGA | |
| | TAAAATGGATGGGTCAATGCCTTCAGAAATGGAATCCAGTGGAAATGATACCAAAGAT | |
| | AATCTTGGTAATTTAAATGTGGCTGATTCATCTCTCCCTGCTGGAAATGAAGTGAGTG | |
| | GCCCTGGCTTTGTTCCTCCACCTCTTGCTTCAATCAGAGGTCCATTGTTTCCAGTGGA | |
| | TACGAGGGGCCCGTTCATGAGAAGAGGACCTCCTTTCCCTCCACCTCCTCCAGGAACC | |
| | ATGTTTGGAGCTTCTCCAGATTATTTTCCACCAAGGGATGTCCCAGGTCCACCACGTG | |
| | CTCCATTTGCAATGAGAAATGTCTGTCCACCGAGGGGTTTTCCTCCTTACCTTCCCCC | |
| | AAGACCTGGATTTTGCCCCCACCCCCACCCCCACAGTGAGTTCCCTTTAGGGTTGAGT | |
| | CTGCCTTCAAATGAGCCTGCTGCTGAAGATCCAGAACCACGGCAAGAAACCTGATAAT | |
| | ATTTTTGCTGTCTTCAAAAGTCATTTTGACTATTCTCATTTTCAGTTGAAGTAACTGC | |
| | TGTTACTTCAGTGATTACACTTTTGCTCAAATTGAA | |
| | ORF Start: ATG at 94 | ORF Stop: TGA at 2488 |
| | SEQ ID NO:54 | 798 aa MW at 90383.6 kD |
| NOV20a, | MEEPRATPQLYLGLVLQLLPRVMAALPEGVRPNSNPYGFPWELVICAAVLGFVAVPFF | |
| CG105463-01 | LWRSFRSVRSRLYVGREKELAIALSGLIEEKCRLLEKFSLVQKEYEGYEVESSLEDAS | |
| Protein Sequence | FEKEATEAQSLEANCEKLNRSNSELEHEILCLEKGIKEEKSKHSEQDEVMADISKKIQ | |
| | SLEDESKSLKSLLTEAKMTFKGFQMNEEKLEIGIQDASSENCQLQESQKQLLQEAEVW | |
| | KEQVSELNKQKITFEDSKVHAEQVLNDKENHIETLTERLLKIKDQAAVLEEDITDDGN | |
| | LELEMNSELKDGAYLDNPPKGALKKLIHAAKLNASLTTLEGERNQFIFSYLKLIKPRK | |
| | SLESILKIFRRNKHLCSRKTHILKVRIRNFNRKLMTELYQENEMKLYRKLIVEENNRL | |
| | EKEKLSKVDEMISHATEELETCRKRAKDLEEELERTILFYQGKIIYHEKKAHDNCLAA | |
| | WTAERNLNDLRKENAHKRQKLAETEFKIKLLEKDPYALDVPNTAFGREHSSYGPSPLG | |
| | RPSSETRAFLYLPTLLEGPLRLSPLLPGGGGRDPRGPGNPLDHQITKERGESSCDRFT | |
| | DPHKAPSDTGPLSPPWEQDRRMMFPPPGQSYPDSALPPQRQDRFYSNSARRSGLAELR | |
| | SFNIPSLDKMDGSMPSEMESSGNDTKDNLGNLNVADSSLPAGNEVSGPGFVPPPLASI | |
| | RGPLFPVDTRGPFMRRGPPFPPPPPGTMFGASPDYFPPRDVPGPPRAPFAMRNVCPPR | |
| | GFPPYLPPRPGFCPHPHPHSEFPLGLSLPSNEPAAEDPEPRQET | |
| | SEQ ID NO:55 | 2483 bp |
| NOV20b, | AGCTGGAATTCGCCCTTCTCGACAGGCCATGGTTACTTTGGCCACTGCCAGAGCAGCC | |
| CG105463-02 | TTGGCACTATGGAGGAGCCTAGGGCTACCCCTCAGCTGTACTTGGGGCTGGTCCTGCA | |
| DNA Sequence | GTTGCTACCCAGGGTTATGGCAGCACTGCCTGAAGGTGTGAGACCAAATTCGAATCCT | |
| | TATGGTTTTCCATGGGAATTGGTGATATGTGCAGCTGTCCTTGGATTTGTTGCTGTTC | |
| | CCTTTTTTTTGTGGAGAAGTTTTAGATCGGTTAGGAGTCGGCTTTATGTGGGAAGAGA | |
| | GAAAGAGCTTGCTATAGCGCTTTCTGGACTAATTGAAGAAAAATGTAGACTACTTGAA | |
| | AAATTTAGCCTTGTTCAAAAAGAGTATGAAGGCTATGAAGTAGAGTCATCTTTAGAGG | |
| | ATGCCAGCTTTGAGAAGGAGGCAACAGAAGCACAAAGTCTGGAGGCAAACTGTGAAAA | |
| | GCTGAACAGGTCCAATTCTGAACTGGAGCATGAAATACTCTGTCTAGAAAAGGGGATA | |
| | AAAGAAGAGAAATCTAAACATTCTGAACAAGATGAGGTGATGGCAGATATTTCCAAAA | |
| | AGATACAGTCTCTAGAAGATGAGTCAAAATCCCTCAAATCACTACTAACTGAAGCTAA | |
| | AATGACCTTCAAGGGATTTCAAATGAATGAAGAAAAACTGGAGATAGGAATACAAGAT | |
| | GCTTCGAGTGAAAATTGTCAACTTCAGGAAAGCCAGAAACAGCTTTTGCAAGAAGCTG | |
| | AAGTATGGAAAGAACAAGTGAGTGAACTTAATAAACAGAAAATAACATTTGAAGACTC | |
| | CAAAGTACACGCAGAACAAGTTCTAAATGATAAAGAAAATCACATCGAGACTCTGACT | |
| | GAACGCTTGCTAAAGATCAAAGATCAGGCTGCTGTGCTGGAAGAAGACATAACGGATG | |
| | ATGGTAACTTGGAATTAGAAATGAACAGTGAATTGAAAGATGGTGCTTACTTAGATAA | |
| | TCCTCCAAAAGGAGCTTTGAAGAAACTGATTCATGCTGCTAAGTTAAATGCTTCTTTA | |
| | ACAACCTTAGAAGGAGAAAGAAACCAATTTATATTCAGTTATCTGAAGTTGATAAAAC | |
| | CAAGGAAGAGCTTAGAGAGCATATTAAAAATCTTCAGACGGAACAAGCATCTTTGCAG | |
| | TCGGAAAACACACATTTTGAAAGTGAGAATCAGAAACTTCAACAGAAAGTTAATGACT | |
| | GAGTTATATCAAGAAAATGAAATGAAACTCTACAGGAAATTAATAGTAGAGGAAAATA | |
| | ACCGGTTAGAGAAAGAGAAACTTTCTAAAGTAGACGAAATGATCAGCCATGCCACTGA | |
| | AGAGCTGGAGACCTGCAGAAAGCGAGCCAAAGATCTTGAAGAAGAACTTGAGAGAACT | |
| | ATTCTTTTTTATCAAGGGAAGATTATATACCATGAGAAAAAAGCACATGATAATTGTT | |
| | TGGCAGCATGGACTGCTGAAAGAAACCTCAATGATTTAAGGAAAGAAAATGCTCACAA | |
| | AAGACAAAAATTAGCTGAAACAGAGTTTAAAATTAAACTTTTAGAAAAAGATCCTTAT | |
| | GCACTTGATGTTCCAAATACAGCATTTGGCAGAGAGCATTCCTCATATGGTCCCTCAC | |
| | CATTGGGTCGGCCTTCATCTGAAACGAGAGCTTTTCTCTATCTTCCGACTTTGTTGGA | |
| | GGGTCCACTGAGACTCTCACCTTTGCTTCCAGGGGGAGGAGGAAGAGGCCCAAGAGGC | |
| | CCAGGGAATCCTCTGGACCACCAGATTACCAAGGAAAGAGGAGAATCAAGCTGTGATA | |
| | GGTTTACTGATCCTCACAAGGCTCCTTCTGACACTGGGCCCCTGTCACCTCCGTGGGA | |
| | ACAGGACCGTAGGATGATGTTTCCTCCACCAGGACAATCATATCCTGATTCAGCTCTT | |
| | CCTCCACAAAGGCAAGACAGATTTTATTCTAATTCTGCTAGACGCTCTGGACTAGCAG | |
| | AACTCAGAAGTTTTAATATACCTTCTTTGGATAAAATGGATGGGTCAATGCCTTCAGA | |
| | AATGGAATCCAGTGGAAATGATACCAAAGATAATCTTGGTAATTTAAATGTGGCTGAT | |
| | TCATCTCTCCCTGCTGGAAATGAAGTGAGTGGCCCTGGCTTTGTTCCTCCACCTCTTG | |
| | CTCCAATCAGAGGTCCGTTGTTTCCAGTGGATACGAGGGGCCCGTTCATGAGAAGAGG | |
| | ACCTCCTTTCCCTCCACCTCCTCCAGGAACCATGTTTGGAGCTTCTCCAGATTATTTT | |
| | CCACCAAGGGATGTCCCAGGTCTACCACGTGCTCCATTTGCAATGAGAAATGTCTGTC | |
| | CACCGAGGGGTTTTCCTCCTTACCTTCCCCCAAGACCTGGATTTTGCCCCCACCCCCA | |
| | CCCCCACATTCTGAAGATAGAGTGAGTTCCCTTTAGGGTTGAGTGCCTTCAATGAGC | |
| | CTGCTGCTGAAGATCCAGAACCACGGCAAGAAACCTGATAATATTTT | |
| | ORF Start: ATG at 67 | ORF Stop: TGA at 2401 |
| | SEQ ID NO:56 | 778 aa MW at 88255.5 kD |

| | |
|---|---|
| NOV20b, | MEEPRATPQLYLGLVLQLLPRVMAALPEGVRPNSNPYGFPWELVICAAVLGFVAVPFF |
| CG105463-02 | LWRSFRSVRSRLYVGREKELAIALSGLIEEKCRLLEKFSLVQKEYEGYEVESSLEDAS |
| Protein Sequence | FEKEATEAQSLEANCEKLNRSNSELEHEILCLEKGIKEEKSKHSEQDEVMADISKKIQ |
| | SLEDESKSLKSLLTEAKMTFKGFQMNEEKLEIGIQDASSENCQLQESQKQLLQEAEVW |
| | KEQVSELNKQKITFEDSKVHAEQVLNDKENHIETLTERLLKIKDQAAVLEEDITDDGN |
| | LELEMNSELKDGAYLDNPPKGALKKLIHAAKLNASLTTLEGERNQFIFSYLKLIKPRK |
| | SLESILKIFRRNKHLCSRKTHILKVRIRNFNRKLMTELYQENEMKLYRKLIVEENNRL |
| | EKEKLSKVDEMISHATEELETCRKRAKDLEEELERTILFYQGKIIYHEKKAHDNCLAA |
| | WTAERNLNDLRKENAHKRQKLAETEFKIKLLEKDPYALDVPNTAFGREHSSYGPSPLG |
| | RPSSETRAFLYLPTLLEGPLRLSPLLPGGGGRGPRGPGNPLDHQITKERGESSCDRFT |
| | DPHKAPSDTGPLSPPWEQDRRMMFPPPGQSYPDSALPPQRQDRFYSNSARRSGLAELR |
| | SFNIPSLDKMDGSMPSEMESSGNDTKDNLGNLNVADSSLPAGNEVSGPGFVPPPLAPI |
| | RGPLFPVDTRGPFMRRGPPFPPPPPGTMFGASPDYFPPRDVPGLPRAPFAMRNVCPPR |
| | GFPPYLPPRPGFCPHPHPHILKIE |

Sequence comparison of the above protein sequences yields the following sequence relationships shown in Table 20B.

TABLE 20B
Comparison of NOV20a against NOV20b.

| | NOV20a Residues/ | Identities/Similarities |
|---|---|---|
| Protein Sequence | Match Residues | for the Matched Region |
| NOV20b | 1 . . . 750 | 662/750 (88%) |
| | 1 . . . 750 | 662/750 (88%) |

Further analysis of the NOV20a protein yielded the following properties shown in Table 20C.

TABLE 20C
Protein Sequence Properties NOV20a

| | |
|---|---|
| PSort | 0.4600 probability located in plasma membrane; |
| analysis: | 0.1000 probability located in |
| | endoplasmic reticulum (membrane); |
| | 0.1000 probability located in |
| | endoplasmic reticulum (lumen); |
| | 0.1000 probability located in outside |
| SignalP | Cleavage site between residues 25 and 26 |
| analysis: | |

A search of the NOV20a protein against the Geneseq database, a proprietary database that contains sequences published in patents and patent publication, yielded several homologous proteins shown in Table 20D.

TABLE 20D
Geneseq Results for NOV20a

| | | NOV20a | Identities/ | |
|---|---|---|---|---|
| | | Residues/ | Similarities for | |
| Geneseq | Protein/Organism/Length [Patent | Match | the Matched | Expect |
| Identifier | #, Date] | Residues | Region | Value |
| AAY77574 | Human cytoskeletal protein | 1 . . . 798 | 638/812 (78%) | 0.0 |
| | (HCYT) (clone 3768043) - Homo | 1 . . . 806 | 683/812 (83%) | |
| | sapiens, 806 aa. [WO200006730- | | | |
| | A2, 10-FEB-2000] | | | |
| ABG05280 | Novel human diagnostic protein | 1 . . . 797 | 639/814 (78%) | 0.0 |
| | #5271 - Homo sapiens, 881 aa. | 59 . . . 867 | 684/814 (83%) | |
| | [WO200175067-A2, 11-OCT-2001] | | | |
| ABG05280 | Novel human diagnostic protein | 1 . . . 797 | 639/814 (78%) | 0.0 |
| | #5271 - Homo sapiens, 881 aa. | 59 . . . 867 | 684/814 (83%) | |
| | [WO200175067-A2, 11-OCT-2001] | | | |
| ABG20258 | Novel human diagnostic protein | 1 . . . 797 | 634/814 (77%) | 0.0 |
| | #20249 - Homo sapiens, 881 aa. | 59 . . . 867 | 681/814 (82%) | |
| | [WO200175067-A2, 11-OCT-2001] | | | |
| ABG20258 | Novel human diagnostic protein | 1 . . . 797 | 634/814 (77%) | 0.0 |
| | #20249 - Homo sapiens, 881 aa. | 59 . . . 867 | 681/814 (82%) | |
| | [WO200175067-A2, 11-OCT-2001] | | | |

In a BLAST search of public sequence databases, the NOV20a protein was found to have homology to the proteins shown in the BLASTP data in Table 20E.

TABLE 20E
Public BLASTP Results for NOV20a

| | | NOV20a | Identities/ | |
|---|---|---|---|---|
| Protein | | Residues/ | Similarities for | |
| Accession | | Match | the Matched | Expect |
| Number | Protein/Organism/Length | Residues | Portion | Value |
| O15320 | Meningioma-expressed antigen 6/11 | 1 . . . 798 | 653/810 (80%) | 0.0 |
| | (MEA6) (MEA11) - Homo sapiens | 1 . . . 804 | 696/810 (85%) | |
| | (Human), 804 aa. | | | |
| Q96SG9 | BA500G10.2 (NOVEL PROTEIN | 1 . . . 798 | 616/805 (76%) | 0.0 |
| | SIMILAR TO MENINGIOMA | 15 . . . 816 | 670/805 (82%) | |
| | EXPRESSED ANTIGEN 6 (MEA6) | | | |
| | AND 11 (MEA11)) - Homo sapiens | | | |
| | (Human), 825 aa (fragment). | | | |
| Q96RT6 | CTAGE-2 - Homo sapiens (Human), | 30 . . . 775 | 605/749 (80%) | 0.0 |
| | 754 aa. | 1 . . . 746 | 642/749 (84%) | |
| O95046 | WUGSC: H_DJ0988G15.3 | 1 . . . 770 | 570/775 (73%) | 0.0 |
| | PROTEIN (DJ1005H11.2) | 1 . . . 775 | 633/775 (81%) | |
| | (WUGSC: H_DJ0988G15.3 | | | |
| | PROTEIN) - Homo sapiens | | | |
| | (Human), 777 aa. | | | |
| AAH26864 | SIMILAR TO MENINGIOMA | 30 . . . 796 | 520/783 (66%) | 0.0 |
| | EXPRESSED ANTIGEN 6 | 1 . . . 778 | 600/783 (76%) | |
| | (COILED-COIL PROLINE-RICH) - | | | |
| | Mus musculus (Mouse), 779 aa. | | | |

PFam analysis predicts that the NOV20a protein contains the domains shown in the Table 20F.

TABLE 20F
Domain Analysis of NOV20a

| | | | |
|---|---|---|---|
| | | Identities/ | |
| Pfam | | Similarities | Expect |
| Domain | NOV20a Match Region | for the Matched Region | Value |

No Significant Matches Found

### Example 21

The NOV21 clone was analyzed, and the nucleotide and encoded polypeptide sequences are shown in Table 21A.

TABLE 21A
NOV21 Sequence Analysis

| | SEQ ID NO:57 | 1269 bp |
|---|---|---|
| NOV21a, | AGCGGGGGGCGCTGCCTCGAGCCTCATGGCTGCCCCTGCTTCCGTCATGGGCCCACTC | |
| CG105491-01 | GGGCCCTCTGCCCTGGGCCTTCTGCTGCTGCTCCTGGTGGTGGCCCCTCCCCGGGTCG | |
| DNA Sequence | CAGCATTGGTCCACAGACAGCCAGAGAACCAGGGAATCTCCCTAACTGGCAGCGTGGC | |
| | CTGTGGTCGGCCCAGCATGGAGGGGAAAATCCTGGGCGGCGTCCCTGCGCCCGAGAGG | |
| | AAGTGGCCGTGGCAGGTCAGCGTGCACTACGCAGGCCTCCACGTCTGCGGCGGCTCCA | |
| | TCCTCAATGAGTACTGGGTGCTGTCAGCTGCGCACTGCTTTCACAGGGACAAGAATAT | |
| | CAAAATCTATGACATGTACGTAGGCCTCGTAAACCTCAGGGTGGCCGGCAACCACACC | |
| | CAGTGGTATGAGGTGAACAGGGTGATCCTGCACCCCACATATGAGATGTACCACCCCA | |
| | TCGGAGGTGACGTGGCCCTGGTGCAGCTGAAGACCCGCATTGTGTTTTCTGAGTCCGT | |
| | GCTCCCGGTTTGCCTTGCAACTCCAGAAGTGAACCTTACCAGTGCCAATTGCTGGGCT | |
| | ACGGGATGGGGACTAGTCTCAAAACAAGGTGAGACCTCAGACGAGCTGCAGGAGGTGC | |
| | AGCTCCCGCTGATCCTGGAGCCCTGGTGCCACCTGCTCTACGGACACATGTCCTACAT | |
| | CATGCCCGACATGCTGTGTGCTGGGGACATCCTGAATGCTAAGACCGTGTGTGAGGGC | |
| | GACTCCGGGGGCCCACTTGTCTGTGAATTCAACCGCAGCTGGTTGCAGATTGGAATTG | |
| | TGAGCTGGGGCCGAGGCTGCTCCAACCCTCTGTACCCTGGAGTGTATGCCAGTGTTTC | |
| | CTATTTCTCAAAATGGATATGTGATAACATAGAAATCACGCCCACTCCTGCTCAGCCA | |
| | GCCCCTGCTCTCTCTCCAGCTCTGGGGCCCACTCTCAGCGTCCTAATGGCCATGCTGG | |
| | CTGGCTGGTCAGTGCTGTGAGGTCAGGATACCCACTCTAGGATTCTCATGGCTGCACA | |
| | CCCTGCCCCAGCCCAGCTGCCTCCAGACCCCTAAGCATCTCCTGTCCTGGCCTCTCTG | |
| | AAGCAGACAAGGGCCACCTATCCCGGGGGTGGATGCTGAGTCCAGGAGGTGATGAGCA | |
| | AGTGTACAAAAGAAAAAAGGGAAGGGGGAGAGGGGCTGGTCAGGGAGAACCCAGCTTG | |
| | GGCAGAGTGCACCTGAGATTTGATAAGATCATTAAATATTTACAAAGCAAA | |
| | ORF Start: ATG at 26 | ORF Stop: TGA at 1004 |
| | SEQ ID NO:58 | 326 aa MW at 35323.8 kD |

| | |
|---|---|
| NOV21a, | MAAPASVMGPLGPSALGLLLLLLVVAPPRVAALVHRQPENQGISLTGSVACGRPSMEG |
| CG105491-01 | KILGGVPAPERKWPWQVSVHYAGLHVCGGSILNEYWVLSAAHCFHRDKNIKIYDMYVG |
| Protein Sequence | LVNLRVAGNHTQWYEVNRVILHPTYEMYHPIGGDVALVQLKTRIVFSESVLPVCLATP |
| | EVNLTSANCWATGWGLVSKQGETSDELQEVQLPLILEPWCHLLYGHMSYIMPDMLCAG |
| | DILNAKTVCEGDSGGPLVCEFNRSWLQIGIVSWGRGCSNPLYPGVYASVSYFSKWICD |
| | NIEITPTPAQPAPALSPALGPTLSVLMAMLAGWSVL |

Further analysis of the NOV21a protein yielded the following properties shown in Table 21B.

TABLE 21B
Protein Sequence Properties NOV21a

| | |
|---|---|
| PSort | 0.7900 probability located in plasma membrane; |
| analysis: | 0.3000 probability located in Golgi body; |
| | 0.2000 probability located in endoplasmic |
| | reticulum (membrane); |
| | 0.1007 probability located in microbody (peroxisome) |
| SignalP | Cleavage site between residues 33 and 34 |
| analysis: | |

A search of the NOV21a protein against the Geneseq database, a proprietary database that contains sequences published in patents and patent publication, yielded several homologous proteins shown in Table 21C.

TABLE 21C
Geneseq Results for NOV21a

| | | NOV21a | Identities/ | |
|---|---|---|---|---|
| | | Residues/ | Similarities for | |
| Geneseq | Protein/Organism/Length [Patent | Match | the Matched | Expect |
| Identifier | #, Date] | Residues | Region | Value |
| AAU82747 | Amino acid sequence of novel | 1 . . . 326 | 326/326 (100%) | 0.0 |
| | human protease #46 - Homo | 1 . . . 326 | 326/326 (100%) | |
| | sapiens, 326 aa. [WO200200860- | | | |
| | A2, 03-JAN-2002] | | | |
| AAB73945 | Human protease T - Homo sapiens, | 13 . . . 288 | 115/286 (40%) | 6e−53 |
| | 290 aa. [WO200116293-A2, | 4 . . . 272 | 152/286 (52%) | |
| | 08-MAR-2001] | | | |
| AAE03821 | Human gene 4 encoded secreted | 13 . . . 288 | 115/286 (40%) | 6e−53 |
| | protein HWHIH10, SEQ ID NO: 67 - | 4 . . . 272 | 152/286 (52%) | |
| | Homo sapiens, 290 aa. | | | |
| | [WO200136440-A1, 25-MAY-2001] | | | |
| AAU12282 | Human PRO4327 polypeptide | 13 . . . 288 | 115/286 (40%) | 6e−53 |
| | sequence - Homo sapiens, 290 aa. | 4 . . . 272 | 152/286 (52%) | |
| | [WO200140466-A2, 07-JUN-2001] | | | |
| AAY73388 | HTRM clone 3376404 protein | 13 . . . 288 | 115/286 (40%) | 6e−53 |
| | sequence - Homo sapiens, 290 aa. | 4 . . . 272 | 152/286 (52%) | |
| | [WO9957144-A2, 11-NOV-1999] | | | |

In a BLAST search of public sequence databases, the NOV21a protein was found to have homology to the proteins shown in the BLASTP data in Table 21D.

TABLE 21D
Public BLASTP Results for NOV21a

| | | NOV21a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9BQR3 | Marapsin precursor (EC 3.4.21.—) - | 13 . . . 288 | 115/286 (40%) | 1e−52 |
| | Homo sapiens (Human), 290 aa. | 4 . . . 272 | 152/286 (52%) | |
| Q9PVX7 | EPIDERMIS SPECIFIC SERINE | 50 . . . 314 | 98/275 (35%) | 5e−52 |
| | PROTEASE - Xenopus laevis | 16 . . . 287 | 159/275 (57%) | |
| | (African clawed frog), 389 aa. | | | |
| AAH24903 | RIKEN CDNA 2010001P08 | 51 . . . 326 | 114/288 (39%) | 2e−51 |
| | GENE - Mus musculus (Mouse), | 45 . . . 329 | 158/288 (54%) | |
| | 331 aa. | | | |
| Q91XC4 | SIMILAR TO DISTAL | 51 . . . 288 | 106/247 (42%) | 6e−51 |
| | INTESTINAL SERINE | 28 . . . 272 | 138/247 (54%) | |
| | PROTEASE - Mus musculus | | | |
| | (Mouse), 310 aa. | | | |
| Q9QYZ9 | DISTAL INTESTINAL SERINE | 51 . . . 288 | 105/247 (42%) | 7e−50 |
| | PROTEASE - Mus musculus | 28 . . . 272 | 137/247 (54%) | |
| | (Mouse), 310 aa. | | | |

PFam analysis predicts that the NOV21a protein contains the domains shown in the Table 21E.

TABLE 21E
Domain Analysis of NOV21a

| | | Identities/ | |
|---|---|---|---|
| Pfam | | Similarities | Expect |
| Domain | NOV21a Match Region | for the Matched Region | Value |
| trypsin | 60 . . . 288 | 87/265 (33%) | 5.3e−72 |
| | | 172/265 (65%) | |

### Example 22

The NOV22 clone was analyzed, and the nucleotide and encoded polypeptide sequences are shown in Table 22A.

TABLE 22A
NOV22 Sequence Analysis

| | |
|---|---|
| SEQ ID NO: 59 | 4131bp |

| | |
|---|---|
| NOV22a, | GTGCCGAGGATGGCCAGGCAGCCACCGCCGCCCTGGATCCATGCAGCCTTCCTCCTCT |
| CG105954-01 | |
| DNA Sequence | GCCTCCTCAGTCTTGGCGGAGCCATCGAAATTCCTATGGTTCCAAGCATTCAGAATGA |
| | GCTGACGCAGCCGCCAACCATCACCAAGCAGTCAGCGAAGGATCACATCGTGGACCCC |
| | CGTGATAACATCCTGATTGAGTGTGAAGCAAAAGGGAACCCTGCCCCCAGCTTCCACT |
| | GGACACGAAACAGCAGATTCTTCAACATCGCCAAGGACCCCCGGGTGTCCATGAGGAG |
| | GAGGTCTGGGACCCTGGTGATTGACTTCCGCAGTGGCGGGCGGCCGGAGGAATATGAG |
| | GGGGAATATCAGTGCTTCGCCCGCAACAAATTTGGCACGGCCCTGTCCAATAGGATCC |
| | GCCTGCAGGTGTCTAAATCTCCTCTGTGGCCCAAGGAAAACCTAGACCCTGTCGTGGT |
| | CCAAGAGGGCGCTCCTTTGACGCTCCAGTGCAACCCCCCGCCTGGACTTCCATCCCCG |
| | GTCATCTTCTGGATGAGCAGCGCCATGGAGCCCATCACCCAAGACAAACGTGTCTCTC |
| | AGGGCCATAACGGAGACCTATACTTCTCCAACGTGATGCTGCAGGACATGCAGACCGA |
| | CTACAGTTGTAACGCCCGCTTCCACTTCACCCACACCATCCAGCAGAAGAACCCTTTC |
| | ACCCTCAAGGTCCTCACCAGTAAGCCTTATAATGACTCGTCCTTAAGAAACCACCCTG |
| | ACATGTACAGTGCCCGAGGAGTTGCAGAAAGAACACCAAGCTTCATGTATCCCCAGGG |
| | CACCGCGAGCAGCCAGATGGTGCTTCGTGGCATGGACCTCCTGCTGGAATGCATCGCC |
| | TCCGGGGTCCCAACACCAGACATCGCATGGTACAAGAAAGGTGGGGACCTCCCATCTG |
| | ATAAGGCCAAGTTTGAGAACTTTAATAAGGCCCTGCGTATCACAAATGTCTCTGAGGA |
| | AGACTCCGGGGAGTATTTCTGCCTGGCCTCCAACAAGATGGGCAGCATCCGGCACACG |
| | ATCTCGGTGAGAGTAAAGGCTGCTCCCTACTGGCTGGACGAACCCAAGAACCTTATTC |
| | TGGCTCCTGGCGAGGATGGGAGACTGGTGTGTCGAGCCAATGGAAACCCCAAACCCAC |
| | TGTCCAGTGGATGGTGAATGGGGAACCTTTGCAAGCGGCACCACCTAACCCAAACCGT |
| | GAGGTGGCCGGAGACACCATCATCTTCCGGGACACCCAGATCAGCAGCAGGGCTGTGT |
| | ACCAGTGCAACACCTCCAACGAGCATGGCTACCTGCTGGCCAACGCCTTTGTCAGTGT |
| | GCTGGATGTGCCGCCTCGGATGCTGTCGCCCCGGAACCAGCTCATTCGAGTGATTCTT |
| | TACAACCGGACGCGGCTGGACTGCCCTTTCTTTGGGTCTCCCATCCCCACACTGCGAT |
| | GGTTTAAGAATGGGCAAGGAAGCAACCTGGATGGTGGCAACTACCATGTTTATGAGAA |
| | CGGCAGTCTGGAAATTAAGATGATCCGCAAAGAGGACCAGGGCATCTACACCTGTGTC |
| | GCCACCAACATCCTGGGCAAAGCTGAAAACCAAGTCCGCCTGGAGGTAAAAGACCCCA |
| | CCAGGATCTACCGGATGCCCGAGGACCAGGTGGCCAGAAGGGGCACCACGGTGCAACT |
| | GGAGTGTCGGGTGAAGCACGACCCCTCCCTGAAACTCACCGTCTACTGGCTGAAGGAT |
| | GACGAGCCGCTCTATATTGGAAACAGGATGAAGAAGGAAGACGACTCCCTGACCATCT |
| | TTGGGGTGGCAGAGCGGGACCAGGGCAGTTACACGTGTGTCGCCAGCACCGAGCTAGA |
| | CCAAGACCTGGCCAAGGCCTACCTCACCGTGCTAGGACGGCCAGACCGGCCCCGGGAC |
| | CTGGAGCTGACCGACCTGGCCGAGAGGAGCGTGCGGCTGACCTGGATCCCCGGGGATG |
| | CTAACAACAGCCCCATCACAGACTACGTCGTCCAGTTTGAAGAAGACCAGTTCCAACC |
| | TGGGGTCTGGCATGACCATTCCAAGTACCCCGGCAGCGTTAACTCAGCCGTCCTCCGG |
| | CTGTCCCCGTATGTCAACTACCAGTTCCGTGTCATTGCCATCAACGAGGTTGGGAGCA |
| | GCCACCCCAGCCTCCCATCCGAGCGCTACCGAACCAGTGGAGCACCCCCCGAGTCCAA |
| | TCCTGGTGACGTGAAGGGAGAGGGGACCAGAAAGAACAACATGGAGATCACGTGGACG |
| | CCCATGAATGCCACCTCGGCCTTTGGCCCCAACCTGCGCTACATTGTCAAGTGGAGGC |
| | GGAGAGAGACTCGAGAGGCCTGGAACAACGTCACAGTGTGGGGCTCTCGCTACGTGGT |
| | GGGGCAGACCCCAGTCTACGTGCCCTATGAGATCCGAGTCCAGGCTGAAAATGACTTC |
| | GGGAAGGGCCCTGAGCCAGAGTCCGTCATCGGTTACTCCGGAGAAGATTATCCCAGGG |
| | CTGCGCCCACTGAAGTTAAAGTCCGAGTCATGAACAGCACAGCCATCAGCCTTCAGTG |
| | GAACCGCGTCTACTCCGACACGGTCCAGGGCCAGCTCAGAGAGTACCGAGCCTACTAC |
| | TGGAGGGAGAGCAGCTTGCTGAAGAACCTGTGGGTGTCTCAGAAGAGACAGCAAGCCA |
| | GCTTCCCTGGTGACCGCCTCCGTGGCGTGGTGTCCCGCCTCTTCCCCTACAGTAACTA |
| | CAAGCTGGAGATGGTTGTGGTCAATGGGAGAGGTGATGGGCCTCGCAGTGAGACCAAG |
| | GAGTTCACCACCCCGGAAGGAGTACCCAGTGCCCCTAGGCGTTTCCGAGTCCGGCAGC |
| | CCAACCTGGAGACAATCAACCTGGAATGGGATCATCCTGAGCATCCAAATGGGATCAT |
| | GATTGGATACACTCTCAAATATGTGGCCTGTACGTTCTCCCCAGTTAACGGGACCAAA |
| | GTAGGAAAGCAGATAGTGGAAAACTTCTCTCCCAATCAGACCAAGTTCACGGTGCAAA |
| | GAACGGACCCCGTGTCACGCTACCGCTTTACCCTCAGCGCCAGGACGCAGGTGGGCTC |
| | TGGGGAAGCCGTCACAGAGGAGTCACCAGCACCCCCGAATGAAGGTAGGTGCATGGCA |
| | GCAGCCCCTGGGGTAAAACCCCCGACTACCGTGGGTGCGACGGGCGCTGTGAGCAGTA |
| | CCGATGCTACTGCCATTGCTGCCACCACCGAAGCCACAACAGTCCCCATCATCCCAAC |
| | TGTCGCACCTACCACCATGGCCACCACCACCACCGTCGCCACAACTACTACAACCACT |
| | GCTGCCGCCACCACCACCACGGAGAGTCCTCCCACCACCACCTCCGGGACTAAGATAC |
| | ACGAATCCGGTACTGCGCATCGCCCATGCTCCCCAGCCCCTGATGAGCAGTCCATATG |
| | GAACGTCACGGTGCTCCCCAACAGTAAATGGGCCAACATCACCTGGAAGCACAATTTC |
| | GGGCCCGGAACTGACTTTGTGGTTGAGTACATCGACAGTAACCATACGAAAAAAACTG |
| | TCCCAGTTAAGGCCCAGGCTCAGCCTATACAGCTGACAGACCTCTATCCCGGGATGAC |
| | ATACACGTTGCGGGTTTATTCCCGGGACAACGAGGGCATCAGCAATCATTCACGGGTT |
| | TGCTTCCGGCCCTCCCCGCCAGCTTACACCAACAACCAAGCGGACATCGCCACCCAGG |
| | GCTGGTTCATTGGGCTTATGTGCGCCATCGCCCTCCTGGTGCTGATCCTGCTCATCGT |
| | CTGTTTCATCAAGAGGAGTCGCGGCGGCAAGTACCCAGTACGAGAAAAGAAGGATGTT |
| | CCCCTTGGCCCTGAAGACCCCAAGGAAGAGGATGGCTCATTTGACTATAGGTCTTTGG |
| | CCAGTGATGAGGACAACAAGCCCCTGCAGGGCAGTCAGACATCTCTGGACGGCACCAT |
| | CAAGCAGCAGGAGAGTGACGACAGCCTGGTGGACTATGGCGAGGGTGGCGAGGGTCAG |
| | TTCAATGAAGACGGCTCCTTCATCGGCCAGTACACGGTCAAAAAGGACAAGGAGGAAA |
| | CAGAGGGCAACGAAAGCTCAGAGGCCACGTCACCTGTCAATGCTATCTACTCTCTGGC |
| | CTAACGGAGCCCA |

| | | |
|---|---|---|
| ORF Start: ATG at 10 | | ORF Stop: TAA at 4120 |
| SEQ ID NO: 60 | 1370 aa | MW at 152752.5kD |

| | |
|---|---|
| NOV22a, | MARQPPPPWIHAAFLLCLLSLGGAIEIPMVPSIQNELTQPPTITKQSAKDHIVDPRDN |
| CG105954-01 | |
| Protein Sequence | ILIECEAKGNPAPSFHWTRNSRFFNIAKDPRVSMRRRSGTLVIDFRSGGRPEEYEGEY |
| | QCFARNKFGTALSNRIRLQVSKSPLWPKENLDPVVVQEGAPLTLQCNPPPGLPSPVIF |
| | WMSSAMEPITQDKRVSQGHNGDLYFSNVMLQDMQTDYSCNARFHFTHTIQQKNPFTLK |
| | VLTSKPYNDSSLRNHPDMYSARGVAERTPSFMYPQGTASSQMVLRGMDLLLECIASGV |
| | PTPDIAWYKKGGDLPSDKAKFENFNKALRITNVSEEDSGEYFCLASNKMGSIRHTISV |
| | RVKAAPYWLDEPKNLILAPGEDGRLVCRANGNPKPTVQWMVNGEPLQAAPPNPNREVA |
| | GDTIIFRDTQISSRAVYQCNTSNEHGYLLANAFVSVLDVPPRMLSPRNQLIRVILYNR |
| | TRLDCPFFGSPIPTLRWFKNGQGSNLDGGNYHVYENGSLEIKMIRKEDQGIYTCVATN |
| | ILGKAENQVRLEVKDPTRIYRMPEDQVARRGTTVQLECRVKHDPSLKLTVYWLKDDEP |
| | LYIGNRMKKEDDSLTIFGVAERDQGSYTCVASTELDQDLAKAYLTVLGRPDRPRDLEL |
| | TDLAERSVRLTWIPGDANNSPITDYVVQFEEDQFQPGVWHDHSKYPGSVNSAVLRLSP |
| | YVNYQFRVIAINEVGSSHPSLPSERYRTSGAPPESNPGDVKGEGTRKNNMEITWTPMN |
| | ATSAFGPNLRYIVKWRRRETREAWNNVTVWGSRYVVGQTPVYVPYEIRVQAENDFGKG |
| | PEPESVIGYSGEDYPRAAPTEVKVRVMNSTAISLQWNRVYSDTVQGQLREYRAYYWRE |
| | SSLLKNLWVSQKRQQASFPGDRLRGVVSRLFPYSNYKLEMVVVNGRGDGPRSETKEFT |
| | TPEGVPSAPRRFRVRQPNLETINLEWDHPEHPNGIMIGYTLKYVACTFSPVNGTKVGK |
| | QIVENFSPNQTKFTVQRTDPVSRYRFTLSARTQVGSGEAVTEESPAPPNEGRCMAAAP |
| | GVKPPTTVGATGAVSSTDATAIAATTEATTVPIIPTVAPTTMATTTTVATTTTTTAAA |
| | TTTTESPPTTTSGTKIHESGTAHRPCSPAPDEQSIWNVTVLPNSKWANITWKHNFGPG |
| | TDFVVEYIDSNHTKKTVPVKAQAQPIQLTDLYPGMTYTLRVYSRDNEGISNHSRVCFR |
| | PSPPAYTNNQADIATQGWFIGLMCAIALLVLILLIVCFIKRSRGGKYPVREKKDVPLG |
| | DGSFIGQYTVKKDKEETEGNESSEATSPVNAIYSLA |

Further analysis of the NOV22a protein yielded the following properties shown in Table 22B.

TABLE 22B
Protein Sequence Properties NOV22a

| | |
|---|---|
| PSort | 0.4600 probability located in plasma membrane; |
| analysis: | 0.1000 probability located in endoplasmic |
| | reticulum (membrane); |
| | 0.1000 probability located in endoplasmic reticulum (lumen); |
| | 0.1000 probability located in outside |
| SignalP | Cleavage site between residues 25 and 26 |
| analysis: | |

A search of the NOV22a protein against the Geneseq database, a proprietary database that contains sequences published in patents and patent publication, yielded several homologous proteins shown in Table 22C.

TABLE 22C
Geneseq Results for NOV22a

| | | NOV22a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAM78714 | Human protein SEQ ID NO 1376 - | 92 . . . 1050 | 928/959 (96%) | 0.0 |
| | Homo sapiens, 937 aa. | 1 . . . 937 | 933/959 (96%) | |
| | [WO200157190-A2, 09-AUG-2001] | | | |
| AAM78715 | Human protein SEQ ID NO 1377 - | 92 . . . 1050 | 928/974 (95%) | 0.0 |
| | Homo sapiens, 952 aa. | 1 . . . 952 | 933/974 (95%) | |
| | [WO200157190-A2, 09-AUG-2001] | | | |
| AAW59994 | Human neural cell adhesion | 15 . . . 1072 | 517/1075 (48%) | 0.0 |
| | molecule splice variant NrCAMvar - | 20 . . . 1082 | 708/1075 (65%) | |
| | Homo sapiens, 1304 aa. | | | |
| | [WO9836062-A1, 20-AUG-1998] | | | |
| AAU10650 | Chicken Nr-CAM protein sequence - | 12 . . . 1054 | 508/1055 (48%) | 0.0 |
| | Gallus sp, 1268 aa. [US6313265- | 12 . . . 1042 | 696/1055 (65%) | |
| | B1, 06-NOV-2001] | | | |
| AAB90717 | Human CO722_1 protein sequence | 15 . . . 1060 | 509/1058 (48%) | 0.0 |
| | SEQ ID 130 - Homo sapiens, 1192 | 20 . . . 1047 | 701/1058 (66%) | |
| | aa. [WO200119988-A1, 22-MAR-2001] | | | |

In a BLAST search of public sequence databases, the NOV22a protein was found to have homology to the proteins shown in the BLASTP data in Table 22D.

TABLE 22D
Public BLASTP Results for NOV22a

| | | NOV22a | | |
|---|---|---|---|---|
| Protein | | Residues/ | Identities/ | |
| Accession | | Match | Similarities for the | Expect |
| Number | Protein/Organism/Length | Residues | Matched Portion | Value |
| O42414 | NEUROFASCIN PRECURSOR - | 13 . . . 1370 | 1026/1372 (74%) | 0.0 |
| | Gallus gallus (Chicken), 1369 | 14 . . . 1369 | 1148/1372 (82%) | |
| | aa. | | | |
| Q91Z60 | NEUROFASCIN 155 KDA | 1 . . . 1042 | 985/1042 (94%) | 0.0 |
| | ISOFORM - Rattus norvegicus | 1 . . . 1031 | 1008/1042 (96%) | |
| | (Rat), 1174 aa. | | | |
| Q9QVN5 | NEUROFASCIN ISOFORM - | 25 . . . 1042 | 965/1019 (94%) | 0.0 |
| | Rattus sp, 1151 aa. | 1 . . . 1008 | 987/1019 (96%) | |
| Q90924 | NEUROFASCIN PRECURSOR - | 13 . . . 1114 | 844/1114 (75%) | 0.0 |
| | Gallus gallus (Chicken), 1272 | 14 . . . 1120 | 949/1114 (84%) | |
| | aa. | | | |
| O94856 | KIAA0756 PROTEIN - Homo | 193 . . . 1050 | 830/858 (96%) | 0.0 |
| | sapiens (Human), 836 aa | 1 . . . 836 | 833/858 (96%) | |
| | (fragment). | | | |

PFam analysis predicts that the NOV22a protein contains the domains shown in the Table 22E.

TABLE 22E
Domain Analysis of NOV22a

| | | Identities/ | | |
|---|---|---|---|---|
| | NOV22a | Similarities for | | |
| Pfam Domain | Match Region | the Matched Region | | Expect Value |
| ig | 56 . . . 120 | 11/68 | (16%) | 0.032 |
| | | 43/68 | (63%) | |
| ig | 155 . . . 215 | 13/62 | (21%) | 0.01 |
| | | 39/62 | (63%) | |
| ig | 278 . . . 335 | 18/61 | (30%) | 4.3e−11 |
| | | 44/61 | (72%) | |
| ig | 368 . . . 427 | 13/63 | (21%) | 2.1e−05 |
| | | 44/63 | (70%) | |
| ig | 462 . . . 520 | 18/62 | (29%) | 1.1e−07 |
| | | 45/62 | (73%) | |
| ig | 553 . . . 611 | 19/60 | (32%) | 5.9e−09 |
| | | 40/60 | (67%) | |
| fn3 | 630 . . . 716 | 28/88 | (32%) | 6.6e−14 |
| | | 64/88 | (73%) | |
| fn3 | 729 . . . 815 | 27/92 | (29%) | 5.9e−07 |
| | | 62/92 | (67%) | |
| fn3 | 827 . . . 922 | 24/97 | (25%) | 3.7e−07 |
| | | 66/97 | (68%) | |
| fn3 | 934 . . . 1026 | 20/97 | (21%) | 2.1e−08 |
| | | 66/97 | (68%) | |
| fn3 | 1134 . . . 1213 | 22/85 | (26%) | 1.5e−08 |
| | | 56/85 | (66%) | |

### Example 23

The NOV23 clone was analyzed, and the nucleotide and encoded polypeptide sequences are shown in Table 23A.

TABLE 23A
NOV23 Sequence Analysis

| | |
|---|---|
| SEQ ID NO: 61 | 2497 bp |

| | |
|---|---|
| NOV23a, | GCCCCGATGGACGCCGCGTTCCTCCTCGTCCTCGGGCTGTTGGCCCAGAGCCTCTGCC |
| CG105963-01 | |
| DNA Sequence | TGTCTTTGGGGGTTCCTGGATGGAGGAGGCCCACCACCCTGTACCCCTGGCGCCGGGC |
| | GCCTGCCCTGAGCCGCGTGCGGAGGGCCTGGGTCATCCCCCCGATCAGCGTATCCGAG |
| | AACCACAAGCGTCTCCCCTACCCCCTGGTTCAGGTGAGCAGGTGGAAGCACCAGTTGG |
| | CCAGCGTCATCTCCAGCATCCAGGGCCCCGGCGTGGATGAGGAGCCCCGGGGCGTCTT |
| | CTCTATCGCCCAGTTCACAGGGAAGGTCTTCCTCAATGCCATGCTGGACCGCGAGAAG |
| | ACTGATCGCTTCAGGCTAAGAGGGTTTGCCCTGGACCTGGGAGGATCCACCCTGGAGG |
| | ACCCCACGGACCTGGAGATTGTAGTTGTGGATCAGAATGACAACCGGCCAGCCTTCCT |
| | GCAGGAGGCGTTCACTGGCCGCGTGCTGGAGGGTGCAGTCCCAGGTACCTATGTGACC |
| | AGGGCAGAGGCCACAGATGCCGACGACCCCGAGACGGACAACGCAGCGCTGCGGTTCT |
| | CCATCCTGCAGCAGGGCAGCCCCGAGCTCTTCAGCATCGACGAGCTCACAGGAGAGAT |
| | CCGCACAGTGCAAGTGGGGCTGGACCGCGAGGTGGTCGCGGTGTACAATCTGACCCTG |
| | CAGGTGGCGGACATGTCTGGAGACGGCCTCACAGCCACTGCTTCAGCCATCATCACCT |
| | TTGATGACATCAATGACAATGCCCCCGAGTTCACCAGGGATGAGTTCTTCATGGAGGC |
| | CATAGAGGCCGTCAGCGGAGTGGATGTGGGACGCCTGGAAGTGGAGGACAGGGACCTG |
| | CCAGGCTCCCCAAACTGGGTGGCCAGGTTCACCATCCTGGAAGGCGACCCCGATGGGC |
| | AGTTCACCATCCGCACGGACCCCAAGACCAACGAGGGTGTTCTGTCCATTGTGAAGGC |
| | CCTGGACTATGAAAGCTGTGAACACTACGAAACTAAAACACACGGGCAGGATAAGACA |
| | GAGAACGCACGGGCAGGGCTGAGGGCTGAGCGGGGCCAGGCCAAGGTCCGCGTGCATG |
| | TGCAGGACACCAACGAGCCCCCCGTGTTCCAGGAGAACCCACTTCGGACCAGCCTAGC |
| | AGAGGGGGCACCCCCAGGCACTCTGGTGGCCACCTTCTCTGCCCGGGACCCTGACACA |
| | GAGCAGCTGCAGAGGCTCAGCTACTCCAAGGACTACGACCCGGAAGACTGGCTGCAAG |
| | TGGACGCAGCCACTGGCCGGATCCAGACCCAGCACGTGCTCAGCCCGGCGTCCCCTTT |
| | CCTCAAGGGCGGCTGGTACAGAGCCATCGTCTTGGCCCAGGATGCCTCCCAGCCCCGC |
| | ACCGCCACCGGCACCCTGTCCATCGAGATCCTGGAGGTGAACGACCATGCACCTGTGC |
| | TGGCCCCGCCGCCGCCGGGCAGCCTGTGCAGCGAGCCACACCAAGGCCCAGGCCTCCT |
| | CCTGGGCGCCACGGATGAGGACCTGCCCCCCCACGGGGCCCCCTTCCACTTCCAGCTG |
| | AGCCCCAGGCTCCCAGAGCTCGGCCGGAACTGGAGCCTCAGCCAGGTCAACCCTCTCT |
| | CCCATCACCGTCTCCACCCAGACCCCCACCTGCCCCATGGCCCCCATTTCATGTCTGT |
| | GGCTCACCAGCTTTTCCCCAGACCCAGCTCCGGAGCCCACAGGCGTGGCCGATGCAGA |
| | AACCTCAGGAAGGTGTGTTGTGAATGTGGGAGGGAGGGTGTGGCGGTCGTGGGCTGTG |
| | CGGGAGTTCTGACTAGGGGAAGTGGGCTCAGCCTGGGCGCACTGGTCATCGTGCTGGC |
| | CAGCGCCCTCCTGCTGCTGGTGCTGGTCCTGCTCGTGGCACTCCGGGCGCGGTTCTGG |
| | AAGCAGTCTCGGGGCAAGGGGCTGCTGCACGGCCCCCAGGACGACCTTCGAGACAATG |
| | TCCTCAACTACGATGAGCAAGGAGGCGGGGAGGAGGACCAGGACGCCTACGACATCAG |
| | CCAGCTGCGTCACCCGACAGCGCTGAGCCTGCCTCTGGGACCGCCGCCACTTCGCAGA |
| | GATGCCCCGCAGGGCCGCCTGCACCCCCAGCCACCCCGAGTGCTGCCCACCAGCCCCC |
| | TGGACATCGCCGACTTCATCAATGATGGCTTGGAGGCTGCAGATAGTGACCCCAGTGT |
| | GCCGCCTTACGACACAGCCCTCATCTATGACTACGAGGGTGACGGCTCGGTGGCGGGG |
| | ACGCTGAGCTCCATCCTGTCCAGCCAGGGCGATGAGGACCAGGACTACGACTACCTCA |
| | GAGACTGGGGGCCCCGCTTCGCCCGGCTGGCAGACATGTATGGGCACCCGTGCGGGTT |
| | GGAGTACGGGGCCAGATGGGACCACCAGGCCAGGGAGGGTCTTTCTCCTGGGGCACTG |
| | CTACCCAGACACAGAGGCCGGACAGCCTGACCCTGGGGCGCAACTGGACATGCCACTC |
| | ccc |

| | | |
|---|---|---|
| ORF Start: ATG at 7 | | ORF Stop: TGA at 2464 |
| SEQ ID NO: 62 | 819 aa | MW at 89687.6kD |

| | |
|---|---|
| NOV23a, | MDAAFLLVLGLLAQSLCLSLGVPGWRRPTTLYPWRRAPALSRVRRAWVIPPISVSENH |
| CG105963-01 | |
| Protein Sequence | KRLPYPLVQVSRWKHQLASVISSIQGPGVDEEPRGVFSIAQFTGKVFLNAMLDREKTD |
| | RFRLRGFALDLGGSTLEDPTDLEIVVVDQNDNRPAFLQEAFTGRVLEGAVPGTYVTRA |
| | EATDADDPETDNAALRFSILQQGSPELFSIDELTGEIRTVQVGLDREVVAVYNLTLQV |
| | ADMSGDGLTATASAIITFDDINDNAPEFTRDEFFMEAIEAVSGVDVGRLEVEDRDLPG |
| | SPNWVARFTILEGDPDGQFTIRTDPKTNEGVLSIVKALDYESCEHYETKTHGQDKTEN |
| | ARAGLRAERGQAKVRVHVQDTNEPPVFQENPLRTSLAEGAPPGTLVATFSARDPDTEQ |
| | LQRLSYSKDYDPEDWLQVDAATGRIQTQHVLSPASPFLKGGWYRAIVLAQDASQPRTA |
| | TGTLSIEILEVNDHAPVLAPPPPGSLCSEPHQGPGLLLGATDEDLPPHGAPFHFQLSP |
| | RLPELGRNWSLSQVNPLSHHRLHPDPHLPHGPHFMSVAHQLFPRPSSGAHRRGRCRNL |
| | RKVCCECGREGVAVVGCAGVLTRGSGLSLGALVIVLASALLLLVLVLLVALRARFWKQ |
| | SRGKGLLHGPQDDLRDNVLNYDEQGGGEEDQDAYDISQLRHPTALSLPLGPPPLRRDA |
| | PQGRLHPQPPRVLPTSPLDIADFINDGLEAADSDPSVPPYDTALIYDYEGDGSVAGTL |
| | SSILSSQGDEDQDYDYLRDWGPRFARLADMYGHPCGLEYGARWDHQAREGLSPGALLP |
| | RHRGRTA |

Further analysis of the NOV23a protein yielded the following properties shown in Table 23B.

TABLE 23B
Protein Sequence Properties NOV23a

| | |
|---|---|
| PSort | 0.6850 probability located in endoplasmic reticulum (mem- |
| analysis: | brane); 0.6400 probability located in plasma membrane; |
| | 0.4600 probability located in Golgi |
| | body; 0.1000 probability located in |
| | endoplasmic reticulum (lumen) |
| SignalP | Cleavage site between residues 22 and 23 |
| analysis: | |

A search of the NOV23a protein against the Geneseq database, a proprietary database that contains sequences published in patents and patent publication, yielded several homologous proteins shown in Table 23C.

TABLE 23C
Geneseq Results for NOV23a

| | | NOV23a | Identities/ | | |
|---|---|---|---|---|---|
| | | Residues/ | Similarities | | |
| Geneseq | Protein/Organism/Length | Match | for the Matched | | Expect |
| Identifier | [Patent #, Date] | Residues | Region | | Value |
| ABG30224 | Novel human diagnostic protein | 1 . . . 819 | 750/820 | (91%) | 0.0 |
| | #30215 - Homo sapiens, 814 aa. | 1 . . . 814 | 766/820 | (92%) | |
| | [WO200175067-A2, Oct. 11, 2001] | | | | |
| ABG30224 | Novel human diagnostic protein | 1 . . . 819 | 750/820 | (91%) | 0.0 |
| | #30215 - Homo sapiens, 814 aa. | 1 . . . 814 | 766/820 | (92%) | |
| | [WO200175067-A2, Oct. 11, 2001] | | | | |
| AAB24089 | Human PRO2198 protein sequence | 1 . . . 819 | 750/820 | (91%) | 0.0 |
| | SEQ ID NO:79 - Homo sapiens, 814 | 1 . . . 814 | 766/820 | (92%) | |
| | aa. [WO200053755-A2, Sep. 14, 2000 | | | | |
| ABB57233 | Mouse ischaemic condition related | 35 . . . 786 | 313/767 | (40%) | e−152 |
| | protein sequence SEQ ID NO:606 - | 149 . . . 902 | 436/767 | (56%) | |
| | Mus musculus, 906 aa. | | | | |
| | [WO200188188-A2, Nov. 22, 2001] | | | | |
| AAY70741 | Human N-cadherin - Homo sapiens, | 40 . . . 786 | 311/762 | (40%) | e−151 |
| | 906 aa. [WO200021555-A1, | 154 . . . 902 | 435/762 | (56%) | |
| | Apr. 20, 2000] | | | | |

In a BLAST search of public sequence databases, the NOV23a protein was found to have homology to the proteins shown in the BLASTP data in Table 23D.

TABLE 23D
Public BLASTP Results for NOV23a

| | | NOV23a | Identities/ | | |
|---|---|---|---|---|---|
| Protein | | Residues/ | Similarities for | | |
| Accession | | Match | the Matched | | Expect |
| Number | Protein/Organism/Length | Residues | Portion | | Value |
| P55291 | Muscle-cadherin precursor (M- | 1 . . . 819 | 750/820 | (91%) | 0.0 |
| | cadherin) (Cadherin-15) (Cadherin- | 1 . . . 814 | 766/820 | (92%) | |
| | 14) - Homo sapiens (Human), 814 | | | | |
| | aa. | | | | |
| P33146 | Muscle-cadherin precursor (M- | 1 . . . 787 | 616/791 | (77%) | 0.0 |
| | cadherin) (Cadherin-15) (Cadherin- | 1 . . . 783 | 662/791 | (82%) | |
| | 14) - Mus musculus (Mouse), 784 | | | | |
| | aa. | | | | |
| IJMSCM | M-cadherin - mouse, 730 aa | 56 . . . 787 | 576/736 | (78%) | 0.0 |
| | (fragment). | 1 . . . 729 | 620/736 | (83%) | |
| Q8UVQ7 | N-CADHERIN - Brachydanio rerio | 39 . . . 786 | 316/762 | (41%) | e−157 |
| | (Zebrafish) (Zebra danjo), 893 aa. | 140 . . . 889 | 443/762 | (57%) | |
| Q90275 | NEURAL-CADHERIN | 39 . . . 786 | 315/763 | (41%) | e−154 |
| | PRECURSOR (N-CADHERIN) - | 29 . . . 779 | 442/763 | (57%) | |
| | Brachydanio rerio (Zebrafish) | | | | |
| | (Zebra danio), 783 aa. | | | | |

PFam analysis predicts that the NOV23a protein contains the domains shown in the Table 23E.

TABLE 23E
Domain Analysis of NOV23a

| | | Identities/ | | |
|---|---|---|---|---|
| | NOV23a | Similarities for | | Expect |
| Pfam Domain | Match Region | the Matched Region | | Value |
| cadherin | 50 . . . 143 | 23/111 | (21%) | 0.011 |
| | | 61/111 | (55%) | |
| cadherin | 157 . . . 251 | 38/108 | (35%) | 8.7e−25 |
| | | 74/108 | (69%) | |
| cadherin | 265 . . . 367 | 34/107 | (32%) | 6.2e−18 |
| | | 74/107 | (69%) | |
| cadherin | 380 . . . 473 | 34/109 | (31%) | 7.7e−20 |
| | | 71/109 | (65%) | |
| Cadherin_C_term | 634 . . . 788 | 83/158 | (53%) | 5.3e−90 |
| | | 146/158 | (92%) | |

### Example 24

The NOV24 clone was analyzed, and the nucleotide and encoded polypeptide sequences are shown in Table 24A.

TABLE 24A
NOV24 Sequence Analysis

| | |
|---|---|
| SEQ ID NO: 63 | 3617 bp |

| | |
|---|---|
| NOV24a, | GAGATGGGACTGCAATAGAAATCCGGGCAGCCCGAAGAGGCACCCAGCGCTCCAGCCA |
| CG105973-01 | |
| DNA Sequence | CCAGCTGGGCCGCCCGGGAGTCCCTGGCTCTAGACCAGCCGCGAGGAGGCGCCGCGAG |
| | AGAGCTGGTCCCTGCCCGCGGCCGGAGGAGGGCTAGAGCCCCTGGGCCAGCCCCCCGA |
| | GCCGGCTGGGCGGGCGGGCGGGTGGGAGCAGACGCCGGGCACTGTCACCACGGGTGCG |
| | CCGAGCGCACCGACCCGGGACACGGGCAGCTGGGGACCGCCAGATTCCACCAGCCCCC |
| | CTTGCCCCGCAGGGGTCCTCGGCTCGCGCTCCTGGGTAGCAGCCACCCACCGGGGCGG |
| | AGGGAGATGTCGCCCGGGGCCAGCCGCGGTCCCCGGGGAAGCCAGGCGCCGCTGATCG |
| | CGCCCCTCTGCTGCGCCGCGGCCGCGCTGGGGATGTTGCTGTGGTCCCCCGCCTGTCA |
| | GGCGTTCAACCTGGACGTGGAAAAGCTCACAGTGTACAGCGGCCCCAAGGGCAGCTAC |
| | TTCGGCTACGCCGTGGACTTCCACATACCCGACGCCCGCACAGCGAGTGTCTTGGTGG |
| | GGGCGCCCAAAGCCAACACCAGCCAGCCCGATATCGTGGAAGGGGGAGCCGTCTATTA |
| | CTGTCCTTGGCCCGCGGAGGGGTCTGCGCAGTGCAGGCAGATACCGTTTGACACCACC |
| | AACAACAGAAAGATCAGAGTTAATGGAACCAAAGAACCTATCGAGTTCAAATCCAATC |
| | AGTGGTTTGGAGCAACAGTGAAAGCTCACAAAGGAAAAGTTGTGGCCTGTGCTCCTTT |
| | ATATCACTGGAGAACTCTTAAACCGACACCAGAAAAGGACCCAGTTGGCACCTGCTAT |
| | GTAGCAATTCAGAACTTCAGCGCCTATGCCGAGTTCTCTCCTTGCCGGAACAGCAATG |
| | CTGATCCGGAAGGCCAGGGTTACTGCCAAGCAGGATTTAGTCTGGATTTTTATAAGAA |
| | TGGAGACCTTATTGTGGGAGGACCTGGGAGTTTCTACTGGCAAGGACAAGTGATCACT |
| | GCCAGTGTTGCAGATATCATTGCAAATTACTCATTCAAGGATATCCTCAGGAAACTGG |
| | CAGGAGAAAAGCAGACGGAAGTGGCTCCAGCTTCCTATGATGACAGTTACCTTGGATA |
| | CTCAGTTGCTGCTGGGGAGTTTACTGGGGATTCTCAGCAAGAATTGGTTGCTGGAATT |
| | CCAAGAGGAGCACAGAATTTTGGATATGTTTCCATCATTAACTCTACGGATATGACGT |
| | TTATTCAGAATTTCACGGGAGAACAGATGGCATCTTATTTTGGATATACCGTTGTCGT |
| | ATCAGATGTTAACAGTGATGGACTGGATGATGTCCTGGTTGGGGCACCTCTCTTTATG |
| | GAACGTGAATTTGAGAGCAACCCCAGAGAAGTAGGGCAAATCTACCTGTATTTGCAAG |
| | TGAGCTCTCTCCTCTTCAGAGACCCCCAGATCCTCACTGGCACCGAGACGTTTGGGAG |
| | ATTCGGTAGTGCTATGGCACACTTAGGAGACCTGAACCAAGATGGATACAATGACATT |
| | GCCATCGGAGTGCCTTTTGCAGGCAAGGATCAAAGAGGCAAAGTGCTCATTTATAATG |
| | GGAACAAAGATGGCTTAAACACCAAGCCTTCCCAAGTTCTGCAAGGAGTGTGGGCCTC |
| | ACATGCTGTCCCTTCCGGATTTGGCTTTACTTTAAGAGGAGATTCAGACATAGACAAG |
| | AATGATTACCCAGATTTGATTGTGGGTGCATTTGGAACAGGAAAAGTCGCTGTTTACA |
| | GAGCAAGACCGGTTGTGACTGTAGATGCCCAGCTTCTGCTGCACCCAATGATTATCAA |
| | TCTTGAAAATAAAACTTGCCAGGTTCCAGACTCTATGACATCTGCTGCCTGCTTTTCT |
| | TTAAGAGTATGTGCATCTGTCACAGGCCAGAGCATTGCAAACACAATAGTCTTGATGG |
| | CAGAGGTGCAATTAGATTCCCTGAAACAGAAAGGAGCTATTAAACGGACGCTCTTCCT |
| | TGATAACCATCAGGCTCATCGCGTCTTCCCTCTTGTGATAAAAAGGCAGAAATCCCAC |
| | CAGTGCCAGGATTTCATCGTTTACCTTCGAGATGAAACTGAATTCCGAGATAAATTAT |
| | CTCCAATCAACATTAGTTTGAATTACAGTTTGGACGAATCCACCTTTAAAGAAGGCCT |
| | GGAAGTGAAACCAATATTGAACTACTACAGAGAAAACATTGTTAGTGAACAGGCTCAC |
| | ATTCTGGTGGACTGTGGAGAAGACAATCTGTGTGTTCCTGACTTGAAGCTGTCGGCTA |
| | GACCAGATAAGCATCAGGTAATCATTGGAGATGAAAATCACCTTATGCTCATAATAAA |
| | TGCAAGAAATGAAGGGGAAGGAGCATATGAAGCTGAACTCTTTGTAATGATACCAGAA |
| | GAGGCAGATTATGTTGGAATCGAACGCAACAACAAGGGATTTCGACCACTGAGCTGTG |
| | AGTACAAGATGGAAAATGTAACCAGGATGGTGGTGTGTGACCTTGGGAACCCTATGGT |
| | GTCTGGAACAAATTATTCCCTGGGCCTCCGATTTGCAGTTCCACGTCTTGAGAAAACA |
| | AACATGAGCATTAACTTCGATCTCCAAATCAGAAGTTCCAACAAGGACAATCCAGACA |
| | GCAATTTTGTGAGCCTGCAAATCAACATCACTGCTGTAGCGCAGGTGGAAATAAGAGG |
| | AGTGTCACACCCTCCGCAGATTGTTCTGCCCATTCATAACTGGGAACCAGAAGAGGAG |
| | CCCCACAAAGAGGAGGAGGTTGGACCATTGGTGGAACATATTTATGAGCTGCACAATA |
| | TTGGACCAAGTACCATCAGTGACACCATCCTGGAGGTGGGCTGGCCTTTCTCTGCCCG |
| | GGATGAATTTCTTCTCTATATTTTCCATATTCAAACTCTGGGACCTCTGCAGTGCCAA |
| | CCAAATCCTAATATCAATCCACAGGATATAAAGCCTGCTGCCTCCCCAGAGGACACCC |
| | CTGAGCTCAGCGCCTTTTTGCGAAACTCTACTATTCCTCATCTTGTCAGGAAGAGGGA |
| | TGTACATGTGGTCGAATTCCACAGACAGAGCCCTGCAAAAATACTGAATTGTACAAAT |
| | ATCGAGTGTTTACAAATCTCCTGTGCAGTGGGACGACTCGAAGGAGGAGAAAGCGCAG |
| | TCCTGAAAGTCAGGTCACGATTATGGGCCCACACCTTCCTCCAGAGAAAAAATGATCC |
| | CTATGCTCTTGCATCCCTGGTGTCCTTTGAAGTTAAGAAGATGCCTTATACAGATCAG |
| | CCAGCAAAACTCCCAGAAGGAAGCATAGTAATTAAGACATCAGTTATTTGGGCAACTC |
| | CGAATGTTTCCTTCTCAATCCCATTATGGGTAATAATACTAGCAATACTTCTTGGATT |
| | GTTGGTTCTCGCCATTTTAACCTTAGCTTTATGGAAGTGTGGATTCTTTGACAGAGCC |
| | AGACCTCCTCAGGAGGACATGACCGACAGGGAACAGCTGACAAATGACAAGACCCCTG |
| | AGGCATGACAAGAAAAAAAAAGAAGACCAAAGACCTCAAACACTGGTCCTGTTCAAAG |
| | AAAAAGAAAGAACATGAGGCC |

| | | |
|---|---|---|
| ORF Start: ATG at 355 | | ORF Stop: TGA at 3544 |
| SEQ ID NO: 64 | 1063 aa | MW at 117472.3kD |

| | |
|---|---|
| NOV24a, | MSPGASRGPRGSQAPLIAPLCCAAAALGMLLWSPACQAFNLDVEKLTVYSGPKGSYFG |
| CG105973-01 | |
| Protein Sequence | YAVDFHIPDARTASVLVGAPKANTSQPDIVEGGAVYYCPWPAEGSAQCRQIPFDTTNN |
| | RKIRVNGTKEPIEFKSNQWFGATVKAHKGKVVACAPLYHWRTLKPTPEKDPVGTCYVA |
| | IQNFSAYAEFSPCRNSNADPEGQGYCQAGFSLDFYKNGDLIVGGPGSFYWQGQVITAS |
| | VADIIANYSFKDILRKLAGEKQTEVAPASYDDSYLGYSVAAGEFTGDSQQELVAGIPR |
| | GAQNFGYVSIINSTDMTFIQNFTGEQMASYFGYTVVVSDVNSDGLDDVLVGAPLFMER |
| | EFESNPREVGQIYLYLQVSSLLFRDPQILTGTETFGRFGSAMAHLGDLNQDGYNDIAI |
| | GVPFAGKDQRGKVLIYNGNKDGLNTKPSQVLQGVWASHAVPSGFGFTLRGDSDIDKND |
| | YPDLIVGAFGTGKVAVYRARPVVTVDAQLLLHPMIINLENKTCQVPDSMTSAACFSLR |
| | VCASVTGQSIANTIVLMAEVQLDSLKQKGAIKRTLFLDNHQAHRVFPLVIKRQKSHQC |
| | QDFIVYLRDETEFRDKLSPINISLNYSLDESTFKEGLEVKPILNYYRENIVSEQAHIL |
| | VDCGEDNLCVPDLKLSARPDKHQVIIGDENHLMLIINARNEGEGAYEAELFVMIPEEA |
| | DYVGIERNNKGFRPLSCEYKMENVTRMVVCDLGNPMVSGTNYSLGLRFAVPRLEKTNM |
| | SINFDLQIRSSNKDNPDSNFVSLQINITAVAQVEIRGVSHPPQIVLPIHNWEPEEEPH |
| | KEEEVGPLVEHIYELHNIGPSTISDTILEVGWPFSARDEFLLYIFHIQTLGPLQCQPN |
| | PNINPQDIKPAASPEDTPELSAFLRNSTIPHLVRKRDVHVVEFHRQSPAKILNCTNIE |
| | CLQISCAVGRLEGGESAVLKVRSRLWAHTFLQRKNDPYALASLVSFEVKKMPYTDQPA |
| | KLPEGSIVIKTSVIWATPNVSFSIPLWVIILAILLGLLVLAILTLALWKCGFFDRARP |
| | PQEDMTDREQLTNDKTPEA |

| | |
|---|---|
| SEQ ID NO: 65 | 3617 bp |

| | |
|---|---|
| NOV24b, | GAGATGGGACTGCAATAGAAATCCGGGCAGCCCGAAGAGGCACCCAGCGCTCCAGCCA |
| CG105973-02 | |
| DNA Sequence | CCAGCTGGGCCGCCCGGGAGTCCCTGGCTCTAGACCAGCCGCGAGGAGGCGCCGCGAG |
| | AGAGCTGGTCCCTGCCCGCGGCCGGAGGAGGGCTAGAGCCCCTGGGCCAGCCCCCCGA |
| | GCCGGCTGGGCGGGCGGGCGGGTGGGAGCAGACGCCGGGCACTGTCACCACGGGTGCG |
| | CCGAGCGCACCGACCCGGGACACGGGCAGCTGGGGACCGCCAGATTCCACCAGCCCCC |
| | CTTGCCCCGCAGGGGTCCTCGGCTCGCGCTCCTGGGTAGCAGCCACCCACCGGGGCGG |
| | AGGGAGATGTCGCCCGGGGCCAGCCGCGGTCCCCGGGGAAGCCAGGCGCCGCTGATCG |
| | CGCCCCTCTGCTGCGCCGCGGCCGCGCTGGGGATGTTGCTGTGGTCCCCCGCCTGTCA |
| | GGCGTTCAACCTGGACGTGGAAAAGCTCACAGTGTACAGCGGCCCCAAGGGCAGCTAC |
| | TTCGGCTACGCCGTGGACTTCCACATACCCGACGCCCGCACAGCGAGTGTCTTGGTGG |
| | GGGCGCCCAAAGCCAACACCAGCCAGCCCGATATCGTGGAAGGGGGAGCCGTCTATTA |
| | CTGTCCTTGGCCCGCGGAGGGGTCCGCGCAGTGCAGGCAGATACCGTTTGACACCACC |
| | AACAACAGAAAGATCAGAGTTAATGGAACCAAAGAACCTATCGAGTTCAAATCCAATC |
| | AGTGGTTTGGAGCAACAGTGAAAGCTCACAAAGGAAAAGTTGTGGCCTGTGCTCCTTT |
| | ATATCACTGGAGAACTCTTAAACCGACACCAGAAAAGGACCCAGTTGGCACCTGCTAT |
| | GTAGCAATTCAGAACTTCAGCGCCTATGCCGAGTTCTCTCCTTGCCGGAACAGCAATG |
| | CTGATCCGGAAGGCCAGGGTTACTGCCAAGCAGGATTTAGTCTGGATTTTTATAAGAA |
| | TGGAGACCTTATTGTGGGAGGACCTGGGAGTTTCTACTGGCAAGGACAAGTGATCACT |
| | GCCAGTGTTGCAGATATCATTGCAAATTACTCATTCAAGGATATCCTCAGGAAACTGG |
| | CAGGAGAAAAGCAGACGGAAGTGGCTCCAGCTTCCTATGATGACAGTTACCTTGGATA |
| | CTCAGTTGCTGCTGGGGAGTTTACTGGGGATTCTCAGCAAGAATTGGTTGCTGGAATT |
| | CCAAGAGGAGCACAGAATTTTGGATATGTTTCCATCATTAACTCTACGGATATGACGT |
| | TTATTCAGAATTTCACGGGAGAACAGATGGCATCTTATTTTGGATATACCGTTGTCGT |
| | ATCAGATGTTAACAGTGATGGACTGGATGATGTCCTGGTTGGGGCACCTCTCTTTATG |
| | GAACGTGAATTTGAGAGCAACCCCAGAGAAGTAGGGCAAATCTACCTGTATTTGCAAG |
| | TGAGCTCTCTCCTCTTCAGAGACCCCCAGATCCTCACTGGCACCGAGACGTTTGGGAG |
| | ATTCGGTAGTGCTATGGCACACTTAGGAGACCTGAACCAAGATGGATACAATGACATT |
| | GCCATCGGAGTGCCTTTTGCAGGCAAGGATCAAAGAGGCAAAGTGCTCATTTATAATG |
| | GGAACAAAGATGGCTTAAACACCAAGCCTTCCCAAGTTCTGCAAGGAGTGTGGGCCTC |
| | ACATGCTGTCCCTTCCGGATTTGGCTTTACTTTAAGAGGAGATTCAGACATAGACAAG |
| | AATGATTACCCAGATTTGATTGTGGGTGCATTTGGAACAGGAAAAGTCGCTGTTTACA |
| | GAGCAAGACCGGTTGTGACTGTAGATGCCCAGCTTCTGCTGCACCCAATGATTATCAA |
| | TCTTGAAAATAAAACTTGCCAGGTTCCAGACTCTATGACATCTGCTGCCTGCTTTTCT |
| | TTAAGAGTATGTGCATCTGTCACAGGCCAGAGCATTGCAAACACAATAGTCTTGATGG |
| | CAGAGGTGCAATTAGATTCCCTGAAACAGAAAGGAGCTATTAAACGGACGCTCTTCCT |
| | TGATAACCATCAGGCTCATCGCGTCTTCCCTCTTGTGATAAAAAGGCAGAAATCCCAC |
| | CAGTGCCAGGATTTCATCGTTTACCTTCGAGATGAAACTGAATTCCGAGATAAATTAT |
| | CTCCAATCAACATTAGTTTGAATTACAGTTTGGACGAATCCACCTTTAAAGAAGGCCT |
| | GGAAGTGAAACCAATATTGAACTACTACAGAGAAAACATTGTTAGTGAACAGGCTCAC |
| | ATTCTGGTGGACTGTGGAGAAGACAATCTGTGTGTTCCTGACTTGAAGCTGTCGGCTA |
| | GACCAGATAAGCATCAGGTAATCATTGGAGATGAAAATCACCTTATGCTCATAATAAA |
| | TGCAAGAAATGAAGGGGAGGGAGCATATGAAGCTGAACTCTTTGTAATGATACCAGAA |
| | GAGGCAGATTATGTTGGAATCGAACGCAACAACAAGGGATTTCGACCACTGAGCTGTG |
| | AGTACAAGATGGAAAATGTAACCAGGATGGTGGTGTGTGACCTTGGGAACCCTATGGT |
| | GTCTGGAACAAATTATTCCCTGGGCCTCCGATTTGCAGTTCCACGTCTTGAGAAAACA |
| | AACATGAGCATTAACTTCGATCTCCAAATCAGAAGTTCCAACAAGGACAATCCAGACA |
| | GCAATTTTGTGAGCCTGCAAATCAACATCACTGCTGTAGCGCAGGTGGAAATAAGAGG |
| | AGTGTCACACCCTCCGCAGATTGTTCTGCCCATTCATAACTGGGAACCAGAAGAGGAG |
| | CCCCACAAAGAGGAGGAGGTTGGACCATTGGTGGAACATATTTATGAGCTGCACAATA |
| | TTGGACCAAGTACCATCAGTGACACCATCCTGGAGGTGGGCTGGCCTTTCTCTGCCCG |
| | GGATGAATTTCTTCTCTATATTTTCCATATTCAAACTCTGGGACCTCTGCAGTGCCAA |
| | CCAAATCCTAATATCAATCCACAGGATATAAAGCCTGCTGCCTCCCCAGAGGACACCC |
| | CTGAGCTCAGCGCCTTTTTGCGAAACTCTACTATTCCTCATCTTGTCAGGAAGAGGGA |
| | TGTACATGTGGTCGAATTCCACAGACAGAGCCCTGCAAAAATACTGAATTGTACAAAT |
| | ATCGAGTGTTTACAAATCTCCTGTGCAGTGGGACGACTCGAAGGAGGAGAAAGCGCAG |
| | TCCTGAAAGTCAGGTCACGATTATGGGCCCACACCTTCCTCCAGAGAAAAAATGATCC |
| | CTATGCTCTTGCATCCCTGGTGTCCTTTGAAGTTAAGAAGATGCCTTATACAGATCAG |
| | CCAGCAAAACTCCCAGAAGGAAGCATAGCAATTAAGACATCAGTTATTTGGGCAACTC |
| | CGAATGTTTCCTTCTCAATCCCATTATGGGTAATAATACTAGCAATACTTCTTGGATT |
| | GTTGGTTCTCGCCATTTTAACCTTAGCTTTATGGAAGTGTGGATTCTTTGACAGAGCC |
| | AGACCTCCTCAGGAGGACATGACCGACAGGGAACAGCTGACAAATGACAAGACCCCTG |
| | AGGCATGACAAGAAAAAAAAAGAAGACCAAAGACCTCAAACACTGGTCCTGTTCAAAG |
| | AAAAAGAAAGAACATGAGGCC |

| | | |
|---|---|---|
| ORF Start: ATG at 355 | | ORF Stop: TGA at 3544 |
| SEQ ID NO: 66 | 1063 aa | MW at 117444.2kD |

| | |
|---|---|
| NOV24b, | MSPGASRGPRGSQAPLIAPLCCAAAALGMLLWSPACQAFNLDVEKLTVYSGPKGSYFG |
| CG105973-02 | |
| Protein Sequence | YAVDFHIPDARTASVLVGAPKANTSQPDIVEGGAVYYCPWPAEGSAQCRQIPFDTTNN |
| | RKIRVNGTKEPIEFKSNQWFGATVKAHKGKVVACAPLYHWRTLKPTPEKDPVGTCYVA |
| | IQNFSAYAEFSPCRNSNADPEGQGYCQAGFSLDFYKNGDLIVGGPGSFYWQGQVITAS |
| | VADIIANYSFKDILRKLAGEKQTEVAPASYDDSYLGYSVAAGEFTGDSQQELVAGIPR |
| | GAQNFGYVSIINSTDMTFIQNFTGEQMASYFGYTVVVSDVNSDGLDDVLVGAPLFMER |
| | EFESNPREVGQIYLYLQVSSLLFRDPQILTGTETFGRFGSAMAHLGDLNQDGYNDIAI |
| | GVPFAGKDQRGKVLIYNGNKDGLNTKPSQVLQGVWASHAVPSGFGFTLRGDSDIDKND |
| | YPDLIVGAFGTGKVAVYRARPVVTVDAQLLLHPMIINLENKTCQVPDSMTSAACFSLR |
| | VCASVTGQSIANTIVLMAEVQLDSLKQKGAIKRTLFLDNHQAHRVFPLVIKRQKSHQC |
| | QDFIVYLRDETEFRDKLSPINISLNYSLDESTFKEGLEVKPILNYYRENIVSEQAHIL |
| | VDCGEDNLCVPDLKLSARPDKHQVIIGDENHLMLIINARNEGEGAYEAELFVMIPEEA |
| | DYVGIERNNKGFRPLSCEYKMENVTRMVVCDLGNPMVSGTNYSLGLRFAVPRLEKTNM |
| | SINFDLQIRSSNKDNPDSNFVSLQINITAVAQVEIRGVSHPPQIVLPIHNWEPEEEPH |
| | KEEEVGPLVEHIYELHNIGPSTISDTILEVGWPFSARDEFLLYIFHIQTLGPLQCQPN |
| | PNINPQDIKPAASPEDTPELSAFLRNSTIPHLVRKRDVHVVEFHRQSPAKILNCTNIE |
| | CLQISCAVGRLEGGESAVLKVRSRLWAHTFLQRKNDPYALASLVSFEVKKMPYTDQPA |
| | KLPEGSIAIKTSVIWATPNVSFSIPLWVIILAILLGLLVLAILTLALWKCGFFDRARP |
| | PQEDMTDREQLTNDKTPEA |

Sequence comparison of the above protein sequences yields the following sequence relationships shown in Table 24B.

TABLE 24B
Comparison of NOV24a against NOV24b.

| | | Identities/ |
|---|---|---|
| | NOV24a Residues/ | Similarities for |
| Protein Sequence | Match Residues | the Matched Region |
| NOV24b | 1 . . . 1063 | 1010/1063 (95%) |
| | 1 . . . 1063 | 1010/1063 (95%) |

Further analysis of the NOV24a protein yielded the following properties shown in Table 24C.

TABLE 24C
Protein Sequence Properties NOV24a

| | |
|---|---|
| PSort | 0.4600 probability located in plasma membrane; 0.1125 |
| analysis: | probability located in microbody (peroxisome); 0.1000 |
| | probability located in endoplasmic reticulum |
| | (membrane); 0.1000 probability located in endoplasmic |
| | reticulum (lumen) |
| SignalP | Cleavage site between residues 39 and 40 |
| analysis: | |

A search of the NOV24a protein against the Geneseq database, a proprietary database that contains sequences published in patents and patent publication, yielded several homologous proteins shown in Table 24D.

TABLE 24D
Geneseq Results for NOV24a

| | | NOV24a | Identities/ | | |
|---|---|---|---|---|---|
| | | Residues/ | Similarities for | | |
| Geneseq | Protein/Organism/Length | Match | the Matched | | Expect |
| Identifier | [Patent #, Date] | Residues | Region | | Value |
| AAM39241 | Human polypeptide SEQ ID NO | 29 . . . 1063 | 1031/1035 | (99%) | 0.0 |
| | 2386 - Homo sapiens, 1035 aa. | 1 . . . 1035 | 1031/1035 | (99%) | |
| | [WO200153312-A1, Jul. 26, 2001] | | | | |
| AAM41027 | Human polypeptide SEQ ID NO | 24 . . . 1063 | 1024/1046 | (97%) | 0.0 |
| | 5958 - Homo sapiens, 1044 aa. | 1 . . . 1044 | 1027/1046 | (97%) | |
| | [WO200153312-A1, Jul. 26, 2001] | | | | |
| ABG18895 | Novel human diagnostic protein | 8 . . . 1055 | 500/1052 | (47%) | 0.0 |
| | #18886 - Homo sapiens, 1061 aa. | 25 . . . 1049 | 692/1052 | (65%) | |
| | [WO200175067-A2, Oct. 11, 2001] | | | | |
| ABG18895 | Novel human diagnostic protein | 8 . . . 1055 | 500/1052 | (47%) | 0.0 |
| | #18886 - Homo sapiens, 1061 aa. | 25 . . . 1049 | 692/1052 | (65%) | |
| | [WO200175067-A2, Oct. 11, 2001] | | | | |
| AAB70508 | Tissue remodeling protein alpha 5 | 34 . . . 1063 | 474/1036 | (45%) | 0.0 |
| | beta 1 integrin (VLA-5) protein - | 37 . . . 1049 | 667/1036 | (63%) | |
| | Mammalian, 1049 aa. | | | | |
| | [WO200111086-A2, Feb. 15, 2001] | | | | |

In a BLAST search of public sequence databases, the NOV24a protein was found to have homology to the proteins shown in the BLASTP data in Table 24E.

TABLE 24E
Public BLASTP Results for NOV24a

| | | NOV24a | Identities/ | | |
|---|---|---|---|---|---|
| Protein | | Residues/ | Similarities for | | |
| Accession | | Match | the Matched | | Expect |
| Number | Protein/Organism/Length | Residues | Portion | | Value |
| P53708 | Integrin alpha-8 - Homo sapiens | 39 . . . 1063 | 1020/1025 | (99%) | 0.0 |
| | (Human), 1025 aa. | 1 . . . 1025 | 1020/1025 | (99%) | |
| O70304 | INTEGRIN ALPHA8 - Mus | 46 . . . 1057 | 910/1012 | (89%) | 0.0 |
| | musculus (Mouse), 1012 aa | 1 . . . 1012 | 972/1012 | (95%) | |
| | (fragment). | | | | |
| P26009 | Integrin alpha-8 precursor - Gallus | 27 . . . 1063 | 797/1037 | (76%) | 0.0 |
| | gallus (Chicken), 1044 aa. | 13 . . . 1044 | 907/1037 | (86%) | |
| P26008 | Integrin alpha-V precursor | 35 . . . 1055 | 493/1024 | (48%) | 0.0 |
| | (Vitronectin receptor alpha | 16 . . . 1022 | 678/1024 | (66%) | |
| | subunit) (CD51) - Gallus gallus | | | | |
| | (Chicken), 1034 aa. | | | | |
| Q9MZD6 | INTEGRIN ALPHA V SUBUNIT | 8 . . . 1055 | 508/1057 | (48%) | 0.0 |
| | PRECURSOR - Bos taurus | 12 . . . 1035 | 692/1057 | (65%) | |
| | (Bovine), 1047 aa. | | | | |

PFam analysis predicts that the NOV24a protein contains the domains shown in the Table 24F.

TABLE 24F
Domain Analysis of NOV24a

| | | Identities/ | | |
|---|---|---|---|---|
| | NOV24a | Similarities for | | Expect |
| Pfam Domain | Match Region | the Matched Region | | Value |
| FG-GAP | 54 . . . 117 | 22/67 | (33%) | 1.4e−15 |
| | | 53/67 | (79%) | |
| FG-GAP | 264 . . . 317 | 19/63 | (30%) | 2.1e−06 |
| | | 42/63 | (67%) | |
| FG-GAP | 318 . . . 383 | 23/66 | (35%) | 1.7e−15 |
| | | 49/66 | (74%) | |
| FG-GAP | 384 . . . 443 | 29/67 | (43%) | 2e−16 |
| | | 53/67 | (79%) | |
| FG-GAP | 447 . . . 501 | 22/66 | (33%) | 7.8e−10 |
| | | 41/66 | (62%) | |
| integrin_A | 1035 . . . 1049 | 10/15 | (67%) | 0.00011 |
| | | 14/15 | (93%) | |

### Example 25

The NOV25 clone was analyzed, and the nucleotide and encoded polypeptide sequences are shown in Table 25A.

TABLE 25A
NOV25 Sequence Analysis

| | |
|---|---|
| SEQ ID NO: 67 | 1524 bp |

| | |
|---|---|
| NOV25a, | TTTGCCATCATGTTGCGGTTGGTGGCAGCTTGCCCTGAGTCATGTGTGGTGTGCACCA |
| CG106915-01 | |
| DNA Sequence | AAGATGTAACCCTCTGTCACCAGCTAACCTATATAGTAGCAGCCCCTATGACCACGAG |
| | GGTTTTAATCATCACCGATGGATATCTCTCCTCTATTGAGAGCACAAACCTGTCTCTC |
| | TTGTTTAATCTTGCCCTGCTCTCCCTAAGCAGAAATGGTATCGAGGATGTTCAGGAAG |
| | ATGCCCTGCATGGGCTTACGATGTTGCGGACCTTGTTGCTGGAGCACAACCAAATATC |
| | CAGCTCTTCGCTCACTGATCACACCTTCAGCAAGCTTCACAGCCTGCAGGTACTGGTG |
| | CTGAGCAATAATGCTCTCCGCACCCTACGAGGGTCTTGGTTCCGAAACACAAGCGGCC |
| | TGACCCGGCTCCAGCTGGATGGGAATCAGATTACTAATCTCACAGACAGTTCTTTCGG |
| | AGGCACGAATCTCCACAGTCTCAGGTATCTGGATTTATCCAACAATTTTATTTCCTAC |
| | ATTGGGAAAGATGCCTTCCGGCCCCTGCCTCAACTACAGGAAGTGGACCTTTCCCGAA |
| | ATAGGTTAGCCCACATGCCGGATGTGTTTACTCCACTGAAGCAGTTAATCCTTCTGAG |
| | CTTAGATAAGAACCAGTGGAGCTGCACTTGTGATCTCCATCCCCTTGCTCGGTTTTTA |
| | AGAAACTACATTAAGTCTTCTGCTCACACGCTCAGGAATGCCAAGGACCTAAATTGCC |
| | AGCCATCTACCGCAGCTGTGGCAGCTGCACAGAGTGTGCTGAGGCTGTCTGAGACCAA |
| | CTGTGATTCCAAAGCTCCCAACTTCACTCTGGTTCTAAAGGACAGAAGTCCCCTCCTC |
| | CCAGGACCAGATGTGGCCCTGCTGACTGTCCTTGGCTTCGCAGGTGCTGTTGGTCTCA |
| | CTTGCCTAGGTTTAGTTGTATTTAACTGGAAACTCCACCAAGGCAAAGCAAATGAACA |
| | CACATCAGAAAACCTTTGTTGCAGAACCTTCGATGAACCCCTGTGTGCTCATGAGGCA |
| | AGAAATTACCACACTAAGGGATACTGCAACTGCCACTTAACTCAGGAAAACGAGATAA |
| | AGGTCATGTCCATTGTGGGGTCCAGAAAAGAAATGCCACTTTTACAGGAAAATAGCCA |
| | TCAAGCAACATCGGCCTCTGAGTCTGCAACCCTTGACAGATCATTTAGAAACCTGAAA |
| | AAGAAAGACCGTGGGGTAGGCAGCACTTTATTTTGCCAGGATGGTAGATTGCTGCATT |
| | CGGAATGTTCAGAGCCTCCTGGAAATATGAGAGCTTTTAATGAAGCAGGCTTACTTAC |
| | AACATATAATCCAAGGAAAGTTCAAAAGCTATGGAATCTTGAGCCTGGAGAAGTCCAG |
| | CCTCAAACTCTGCAACACCATATAATAAGAACAGAAGATATCAGCAGTGACATATTTA |
| | GAAGAAGATATGCAACACCCGCTTCAGCCTTGGCAGGAGAAAGTCTTGAGAAGCGTTT |
| | AACAAATGAATCATGA |

| | | |
|---|---|---|
| ORF Start: ATG at 10 | | ORF Stop: TGA at 1522 |
| SEQ ID NO: 68 | 504 aa | MW at 56079.2kD |

| | |
|---|---|
| NOV25a, | MLRLVAACPESCVVCTKDVTLCHQLTYIVAAPMTTRVLIITDGYLSSIESTNLSLLFN |
| G106915-01 | |
| Protein Sequence | LALLSLSRNGIEDVQEDALHGLTMLRTLLLEHNQISSSSLTDHTFSKLHSLQVLVLSN |
| | NALRTLRGSWFRNTSGLTRLQLDGNQITNLTDSSFGGTNLHSLRYLDLSNNFISYIGK |
| | DAFRPLPQLQEVDLSRNRLAHMPDVFTPLKQLILLSLDKNQWSCTCDLHPLARFLRNY |
| | IKSSAHTLRNAKDLNCQPSTAAVAAAQSVLRLSETNCDSKAPNFTLVLKDRSPLLPGP |
| | DVALLTVLGFAGAVGLTCLGLVVFNWKLHQGKANEHTSENLCCRTFDEPLCAHEARNY |
| | HTKGYCNCHLTQENEIKVMSIVGSRKEMPLLQENSHQATSASESATLDRSFRNLKKKD |
| | RGVGSTLFCQDGRLLHSECSEPPGNMRAFNEAGLLTTYNPRKVQKLWNLEPGEVQPQT |
| | LQHHIIRTEDISSDIFRRRYATPASALAGESLEKRLTNES |

Further analysis of the NOV25a protein yielded the following properties shown in Table 25B.

TABLE 25B
Protein Sequence Properties NOV25a

| | |
|---|---|
| analysis: | probability located in plasma membrane; 0.3000 |
| | probability located in microbody (peroxisome); |
| | 0.2622 probability located in mitochondrial matrix |
| | space |
| SignalP | No Known Signal Sequence Predicted |
| analysis: | |

A search of the NOV25a protein against the Geneseq database, a proprietary database that contains sequences published in patents and patent publication, yielded several homologous proteins shown in Table 25C.

TABLE 25C
Geneseq Results for NOV25a

| | | NOV25a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAU83655 | Human PRO protein, Seq ID No: | 3 . . . 237 | 79/264 (29%) | 5e−19 |
| | 128 - Homo sapiens, 473 aa. | 22 . . . 283 | 121/264 (44%) | |
| | [WO200208288-A2, 31-JAN-2002] | | | |
| AAB49891 | Human PRO526 protein sequence - | 3 . . . 237 | 79/264 (29%) | 5e−19 |
| | Homo sapiens, 473 aa. | 22 . . . 283 | 121/264 (44%) | |
| | [WO200070050-A1, 23-NOV-2000] | | | |
| AAB50908 | Human PRO526 protein - Homo | 3 . . . 237 | 79/264 (29%) | 5e−19 |
| | sapiens, 473 aa. [WO200073452- | 22 . . . 283 | 121/264 (44%) | |
| | A2, 07-DEC-2000] | | | |
| AAU04589 | Human Nogo receptor - Homo | 3 . . . 237 | 79/264 (29%) | 5e−19 |
| | sapiens, 473 aa. [WO200151520- | 22 . . . 283 | 121/264 (44%) | |
| | A2, 19-JUL-2001] | | | |
| AAU12362 | Human PRO526 polypeptide | 3 . . . 237 | 79/264 (29%) | 5e−19 |
| | sequence - Homo sapiens, 473 aa. | 22 . . . 283 | 121/264 (44%) | |
| | [WO200140466-A2, 07-JUN-2001] | | | |

In a BLAST search of public sequence databases, the NOV25a protein was found to have homology to the proteins shown in the BLASTP data in Table 25D.

TABLE 25D
Public BLASTP Results for NOV25a

| | | NOV25a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9BGY6 | HYPOTHETICAL 56.5 KDA | 1 . . . 504 | 478/504 (94%) | 0.0 |
| | PROTEIN - Macaca fascicularis | 1 . . . 504 | 481/504 (94%) | |
| | (Crab eating macaque) | | | |
| | (Cynomolgus monkey), 510 aa. | | | |
| Q961X3 | GH01279P - Drosophila | 45 . . . 233 | 68/212 (32%) | 3e−21 |
| | melanogaster (Fruit fly), 615 aa. | 313 . . . 522 | 103/212 (48%) | |
| Q9N0E3 | UNNAMED PROTEIN PRODUCT - | 3 . . . 237 | 82/264 (31%) | 1e−19 |
| | Macaca fascicularis (Crab eating | 22 . . . 283 | 125/264 (47%) | |
| | macaque) (Cynomolgus monkey), | | | |
| | 473 aa. | | | |
| Q9VZ84 | CG7509 PROTEIN - Drosophila | 45 . . . 233 | 69/230 (30%) | 6e−19 |
| | melanogaster (Fruit fly), 633 aa. | 313 . . . 540 | 103/230 (44%) | |
| Q9BZR6 | NOGO RECEPTOR - Homo | 3 . . . 237 | 79/264 (29%) | 1e−18 |
| | sapiens (Human), 473 aa. | 22 . . . 283 | 121/264 (44%) | |

PFam analysis predicts that the NOV25a protein contains the domains shown in the Table 25E.

TABLE 25E
Domain Analysis of NOV25a

| | NOV25a | Identities/Similarities | Expect |
|---|---|---|---|
| Pfam Domain | Match Region | for the Matched Region | Value |
| LRR | 58 . . . 81 | 7/25 (28%) | 0.3 |
| | | 19/25 (76%) | |
| LRR | 108 . . . 131 | 10/25 (40%) | 0.11 |
| | | 19/25 (76%) | |
| LRR | 132 . . . 155 | 8/25 (32%) | 0.7 |
| | | 18/25 (72%) | |
| LRR | 158 . . . 181 | 11/25 (44%) | 0.00021 |
| | | 19/25 (76%) | |
| LRR | 182 . . . 204 | 10/25 (40%) | 0.093 |
| | | 18/25 (72%) | |
| LRRCT | 214 . . . 270 | 15/63 (24%) | 0.046 |
| | | 42/63 (67%) | |

### Example 26

The NOV26 clone was analyzed, and the nucleotide and encoded polypeptide sequences are shown in Table 26A.

TABLE 26A
NOV26 Sequence Analysis

| | |
|---|---|
| SEQ ID NO: 69 | 3757 bp |

| | |
|---|---|
| NOV26a, | CTCTTTGCCATCATGTTGCGGTTGGTGGCAGCTTGCCCTGAGTCATGTGTGGTGTGCA |
| CG106924-01 | |
| DNA Sequence | CCAAAGATGTAACCCTCTGTCACCAGCTAACCTATATAGTAGCAGCCCCTATGACCAC |
| | GAGGGTTTTAATCATCACCGATGGATATCTCTCCTCTATTGAGAGCACAAACCTGTCT |
| | CTCTTGTTTAATCTTGCCCTGCTCTCCCTAAGCAGAAATGGTATCGAGGATGTTCAGG |
| | AAGATGCCCTGCATGGGCTTACGATGTTGCGGACCTTGTTGCTGGAGCACAACCAAAT |
| | ATCCAGCTCTTCGCTCACTGATCACACCTTCAGCAAGCTTCACAGCCTGCAGGTACTG |
| | GTGCTGAGCAATAATGCTCTCCGCACCCTACGAGGGTCTTGGTTCCGAAACACAAGCG |
| | GCCTGACCCGGCTCCAGCTGGATGGGAATCAGATTACTAATCTCACAGACAGTTCTTT |
| | CGGAGGCACGAATCTCCACAGTCTCAGGTATCTGGATTTATCCAACAATTTTATTTCC |
| | TACATTGGGAAAGATGCCTTCCGGCCCCTGCCTCAACTACAGGAAGTGGACCTTTCCC |
| | GAAATAGGTTAGCCCACATGCCGGATGTGTTTACTCCACTGAAGCAGTTAATCCTTCT |
| | GAGCTTAGATAAGAACCAGTGGAGCTGCACTTGTGATCTCCATCCCCTTGCTCGGTTT |
| | TTAAGAAACTACATTAAGTCTTCTGCTCACACGCTCAGGAATGCCAAGGACCTAAATT |
| | GCCAGCCATCTACCGCAGCTGTGGCAGCTGCACAGAGTGTGCTGAGGCTGTCTGAGAC |
| | CAACTGTGATTCCAAAGCTCCCAACTTCACTCTGGTTCTAAAGGACAGAAGTCCCCTC |
| | CTCCCAGGACCAGATGTGGCCCTGCTGACTGTCCTTGGCTTCGCAGGTGCTGTTGGTC |
| | TCACTTGCCTAGGTTTAGTTGTATTTAACTGGAAACTCCACCAAGGCAAAGCAAATGA |
| | ACACACATCAGAAAACCTTTGTTGCAGAACCTTCGATGAACCCCTGTGTGCTCATGAG |
| | GCAAGAAATTACCACACTAAGGGATACTGCAACTGCCACTTAACTCAGGAAAACGAGA |
| | TAAAGGTCATGTCCATTGTGGGGTCCAGAAAAGAAATGCCACTTTTACAGGAAAATAG |
| | CCATCAAGCAACATCGGCCTCTGAGTCTGCAACCCTTGACAGATCATTTAGAAACCTG |
| | AAAAAGAAAGACCGTGGGGTAGGCAGCACTTTATTTTGCCAGGATGGTAGATTGCTGC |
| | ATTCGGAATGTTCAGAGCCTCCTGGAAATATGAGAGCTTTTAATGAAGCAGGCTTACT |
| | TACAACATATAATCCAAGGAAAGTTCAAAAGCTATGGAATCTTGAGCCTGGAGAAGTC |
| | CAGCCTCAAACTCTGCAACACCATATAATAAGAACAGAAGATATCAGCAGTGACATAT |
| | TTAGAAGAAGATATGCAACACCCGCTTCAGCCTTGGCAGGAGAAAGTCTTGAGAAGCG |
| | TTTAACAAATGAATCATGGCAGCCTCCAATAGAAAAAGAAGACAATGGCTTACACCCT |
| | CACAGGCAAAGACATTTTATTACAAGCTCATCATCCAAGCCTTGTGAGCCTGAGGAAC |
| | ACTATGTACAAAATATCGTACAAAAAAATAGATCAAAATATGATGATCCTTGTGGACT |
| | GTTAAAACAGAGCAAACCTAGGTATTTTCAGCCAAACAATTCTCTTATCTGTAAATAT |
| | GTGCCCTGTGAGCAATTTGAAGATTACATGAAAGAAAAGAAGCCAAATCGTAGACAAC |
| | ACTCAAAGCCTGAGAAAGAGCAAATCCAAATTAACAGTGCAATAGAAAAATTTCTTAT |
| | GAGTGAGGACAACATAGATTTATCAGGATTATCAACAAAAACCAAGAAAGCATATTCC |
| | CCAAAGAGGGTTATCTTCCATGATCCTGATTTAGTAGAAATAAATAGGTCGATGATGT |
| | CACCCAAAATATCAACCCCTTGGAAACGACAGAAAAATCAAAGTAACCAACTGACTAA |
| | GTTGGATGTTAAAAAATTTAGCAACACTGGGGAGAGAAACAAAGGAGAAAAATGGTTT |
| | ACTAATTCATGGGTTCTGAAAAGGAAGAGAACCCCTCAGTCTGACCTCAAAGGGAAAA |
| | TTAAAGGACAAAACTTAAAATTAAATTTACATCCTTTTAGAAAAGTCAGAGTCCATCC |
| | AGAAAAATCCTTGTCAAGTCTCCCAAAGCAATGCAAGCAGGTATTGTTGCCTCCTAAG |
| | AAATTATCCAAAACTTCTGAGACAGAAGCCAAAATAAATACTGTGTGTTCTGCAGATT |
| | TTCTTCAACAGTCAGAGAGTAGCAACTATGTTAGACTCACTTCAAAGAGGCTGCCTCT |
| | GAAACATGACTCAAAGCAGACCCCATATTATCAACGAAACACTAAACGTGCCCCCCTG |
| | CTCAGTGCTAACAACTTGCGTGTAGTCAACCAGAGCTCTATAGAAAGCAGCTGTTACT |
| | CAGCTGGCCACATTCCTGATGGAAACACATCAAAATTGCCCCAACCTACACCCACTGA |
| | TGCTGAGCACAGGCACTCACATTCTCAATTCTCAACTGAGCAAATGGAAGATGCAACT |
| | CAGCTTGAATCAAAAGTGCTTAGTTATTTAGCAACTACTTGGGAAAATACAGGAAGTG |
| | ATGTTTTACCATTCCAACATTCCAGGAGGGCTACTGACCAAGGGACAACGGAGTCCAC |
| | TGAGCACATGGGACAGAATGTATCAAAGACCAGTGAGTTAAATCAGTTTTCTTTGTCC |
| | CCGAGGAATCAAACACAACTTTTAGATGCTCACAAGACTGACAGCTACAACAAGGAAT |
| | ACACTTTAGACCAAAATGAAGGCTTACAACACAGAGAGCAAAATTCAAGTCATGCACA |
| | GCTTGAAAATAAAGAAAAAACATTAATGACAAAACCCCAAATACCACATCAAATTGTG |
| | GAAAATTGTATTATGGATAAGGAAGAAAATGATGTAGAAAAAAAACTTTCAAAAACAG |
| | AAACTTATGATTCCTCTCTCATTCCCCAAACACAATCCAAGAACAACCTATCATTTAT |
| | GAAGACAAATTCAATTCCATACCAAAATAGAATAGAACTTCCCAAGGATATCAGTACT |
| | TCTCCTGTTAGTAGTCAAGCCGTTTGGCACCTAACCAATAGTAGCGAAAAAGGAATTG |
| | ACAGCACAAATGCATTGCCCAGAAATGACGGCACTGAAGCACTAGAGATAAAAATAGT |
| | AGGGAAAGAAGAGAAAAATATGCTTGATGAAAGCAAGACAGATTCTAGTATGTTAACT |
| | CAGATCTCACAAATGACCTTAAAAGGCATCACAAAAGAAAGGCAGCAAACTTGGGAAA |
| | ATGGAACAAGTGAAAAATATATATTACATGATGCAAGCTCTGCCGAGGAGACCATTAC |
| | AGCTAAAGATTTAAGTATCACAAGTTCCCATGAAACCCAAAATAGAATACTTTGCAGT |
| | GAAGTAGATCCTGAAGTTAACAGTAATGTACATAATTTTAGAGAAGTTCAAAATATTC |
| | AACCAGATAAAGATAGGGCACATAAAGAAGGCGCAATGACAGTGGAGACACATGAAGC |
| | GCTTTCCTTCTTACCAGGGTTAAAAGACAGTTTTGAGGCAGAAAATGAGGTGTTTTTA |
| | GTTCCTAGCAGAATAAATGAAGCTGAAAACTCTGCTCCAAAACCTGTACTGTATCCAC |
| | CATCTGCTGAATATGCTACTACATCACCTTTAGAAACAGAATAAA |

| | | |
|---|---|---|
| ORF Start: ATG at 13 | | ORF Stop: TAA at 3754 |
| SEQ ID NO: 70 | 1247 aa | MW at 140902.2 kD |

| | |
|---|---|
| NOV26a, | MLRLVAACPESCVVCTKDVTLCHQLTYIVAAPMTTRVLIITDGYLSSIESTNLSLLFN |
| CG106924-01 | |
| Protein Sequence | LALLSLSRNGIEDVQEDALHGLTMLRTLLLEHNQISSSSLTDHTFSKLHSLQVLVLSN |
| | NALRTLRGSWFRNTSGLTRLQLDGNQITNLTDSSFGGTNLHSLRYLDLSNNFISYIGK |
| | DAFRPLPQLQEVDLSRNRLAHMPDVFTPLKQLILLSLDKNQWSCTCDLHPLARFLRNY |
| | IKSSAHTLRNAKDLNCQPSTAAVAAAQSVLRLSETNCDSKAPNFTLVLKDRSPLLPGP |
| | DVALLTVLGFAGAVGLTCLGLVVFNWKLHQGKANEHTSENLCCRTFDEPLCAHEARNY |
| | HTKGYCNCHLTQENEIKVMSIVGSRKEMPLLQENSHQATSASESATLDRSFRNLKKKD |
| | RGVGSTLFCQDGRLLHSECSEPPGNMRAFNEAGLLTTYNPRKVQKLWNLEPGEVQPQT |
| | LQHHIIRTEDISSDIFRRRYATPASALAGESLEKRLTNESWQPPIEKEDNGLHPHRQR |
| | HFITSSSSKPCEPEEHYVQNIVQKNRSKYDDPCGLLKQSKPRYFQPNNSLICKYVPCE |
| | QFEDYMKEKKPNRRQHSKPEKEQIQINSAIEKFLMSEDNIDLSGLSTKTKKAYSPKRV |
| | IFHDPDLVEINRSMMSPKISTPWKRQENQSNQLTKLDVKKFSNTGERNKGEKWFTNSW |
| | VLKRKRTPQSDLKGKIKGQNLKLNLHPFRKVRVHPEKSLSSLPKQCKQVLLPPKKLSK |
| | TSETEAKINTVCSADFLQQSESSNYVRLTSKRLPLKHDSKQTPYYQRNTKRAPLLSAN |
| | NLRVVNQSSIESSCYSAGHIPDGNTSKLPQPTPTDAEHRHSHSQFSTEQMEDATQLES |
| | KVLSYLATTWENTGSDVLPFQHSRRATDQGTTESTEHMGQNVSKTSELNQFSLSPRNQ |
| | TQLLDAHKTDSYNKEYTLDQNEGLQHREQNSSHAQLENKEKTLMTKPQIPHQIVENCI |
| | MDKEENDVEKKLSKTETYDSSLIPQTQSKNNLSFMKTNSIPYQNRIELPKDISTSPVS |
| | SQAVWHLTNSSEKGIDSTNALPRNDGTEALEIKIVGKEEKNMLDESKTDSSMLTQISQ |
| | MTLKGITKERQQTWENGTSEKYILHDASSAEETITAKDLSITSSHETQNRILCSEVDP |
| | EVNSNVHNFREVQNIQPDKDRAHKEGAMTVETHEALSFLPGLKDSFEAENEVFLVPSR |
| | INEAENSAPKPVLYPPSAEYATTSPLETE |

| | |
|---|---|
| SEQ ID NO: 71 | 645 bp |

| | |
|---|---|
| NOV26b, | GGATCCGCCCTGCTCTCCCTAAGCAGAAATGGTATCGAGGATGTTCAGGAAGATGCCC |
| 210062144 DNA | |
| Sequence | TGCATGGGCTTACGATGTTGCGGACCTTGTTGCTGGAGCACAACCAAATATCCAGCTC |
| | TTCGCTCACTGATCACACCTTCAGCAAGCTTCACAGCCTGCAGGTACTGGTGCTGAGC |
| | AATAATGCTCTCCGCACCCTACGAGGGTCTTGGTTCCGAAACACAAGCGGCCTGACCC |
| | GGCTCCAGCTGGATGGGAATCAGATTACTAATCTCACAGACAGTTCTTTCGGAGGCAC |
| | GAATCTCCACAGTCTCAGGTATCTGGATTTATCCAACAATTTTATTTCCTACATTGGG |
| | AAAGATGCCTTCCGGCCCCTGCCTCAACTACAGGAAGTGGACCTTTCCCGAAATAGGT |
| | TAGCCCACATGCCGGATGTGTTTACTCCACTGAAGCAGTTAATCCTTCTGAGCTTAGA |
| | TAAGAACCAGTGGAGCTGCACTTGTGATCTCCATCCCCTTGCTCGGTTTTTAAGAAAC |
| | TACATTAAGTCTTCTGCTCACACGCTCAGGAATGCCAAGGACCTAAATTGCCAGCCAT |
| | CTACCGCAGCTGTGGCAGCTGCACAGAGTGTGCTGAGGCTGTCTGAGACCAACTGTGA |
| | TCTCGAG |

| | | |
|---|---|---|
| ORF Start: at 1 | | ORF Stop: end of sequence |
| SEQ ID NO: 72 | 215 aa | MW at 23982.8kD |

| | |
|---|---|
| NOV26b, | GSALLSLSRNGIEDVQEDALHGLTMLRTLLLEHNQISSSSLTDHTFSKLHSLQVLVLS |
| 210062144 | |
| Protein Sequence | NNALRTLRGSWFRNTSGLTRLQLDGNQITNLTDSSFGGTNLHSLRYLDLSNNFISYIG |
| | KDAFRPLPQLQEVDLSRNRLAHMPDVFTPLKQLILLSLDKNQWSCTCDLHPLARFLRN |
| | YIKSSAHTLRNAKDLNCQPSTAAVAAAQSVLRLSETNCDLE |

Sequence comparison of the above protein sequences yields the following sequence relationships shown in Table 26B.

TABLE 26B
Comparison of NOV26a against NOV26b.

| | NOV26a Residues/ | Identities/Similarities |
|---|---|---|
| Protein Sequence | Match Residues | for the Matched Region |
| NOV26b | 60 . . . 270 | 199/211 (94%) |
| | 3 . . . 213 | 199/211 (94%) |

Further analysis of the NOV26a protein yielded the following properties shown in Table 26C.

TABLE 26C
Protein Sequence Properties NOV26a

| | |
|---|---|
| PSort | 0.8524 probability located in mitochondrial inner |
| analysis: | membrane; 0.6000 probability located in endoplasmic |
| | reticulum (membrane); 0.3000 probability |
| | located in microbody (peroxisome); 0.2622 |
| | probability located in mitochondrial matrix space |
| SignalP | No Known Signal Sequence Predicted |
| analysis: | |

A search of the NOV26a protein against the Geneseq database, a proprietary database that contains sequences published in patents and patent publication, yielded several homologous proteins shown in Table 26D.

TABLE 26D
Geneseq Results for NOV26a

| | | NOV26a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAU83655 | Human PRO protein, Seq ID No: | 3 . . . 237 | 79/264 (29%) | 1e−18 |
| | 128 - Homo sapiens, 473 aa. | 22 . . . 283 | 121/264 (44%) | |
| | [WO200208288-A2, 31-JAN-2002] | | | |
| AAB49891 | Human PRO526 protein sequence - | 3 . . . 237 | 79/264 (29%) | 1e−18 |
| | Homo sapiens, 473 aa. | 22 . . . 283 | 121/264 (44%) | |
| | [WO200070050-A1, 23-NOV-2000] | | | |
| AAB50908 | Human PRO526 protein - Homo | 3 . . . 237 | 79/264 (29%) | 1e−18 |
| | sapiens, 473 aa. [WO200073452- | 22 . . . 283 | 121/264 (44%) | |
| | A2, 07-DEC-2000] | | | |
| AAU04589 | Human Nogo receptor - Homo | 3 . . . 237 | 79/264 (29%) | 1e−18 |
| | sapiens, 473 aa. [WO200151520- | 22 . . . 283 | 121/264 (44%) | |
| | A2, 19-JUL-2001] | | | |
| AAU12362 | Human PRO526 polypeptide | 3 . . . 237 | 79/264 (29%) | 1e−18 |
| | sequence - Homo sapiens, 473 aa. | 22 . . . 283 | 121/264 (44%) | |
| | [WO200140466-A2, 07-JUN-2001] | | | |

In a BLAST search of public sequence databases, the NOV26a protein was found to have homology to the proteins shown in the BLASTP data in Table 26E.

TABLE 26E
Public BLASTP Results for NOV26a

| | | NOV26a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9BGY6 | HYPOTHETICAL 56.5 KDA | 1 . . . 504 | 478/504 (94%) | 0.0 |
| | PROTEIN - Macaca fascicularis | 1 . . . 504 | 481/504 (94%) | |
| | (Crab eating macaque) | | | |
| | (Cynomolgus monkey), 510 aa. | | | |
| Q961X3 | GH01279P - Drosophila | 45 . . . 233 | 68/212 (32%) | 9e−21 |
| | melanogaster (Fruit fly), 615 aa. | 313 . . . 522 | 103/212 (48%) | |
| Q9N0E3 | UNNAMED PROTEIN PRODUCT - | 3 . . . 237 | 82/264 (31%) | 3e−19 |
| | Macaca fascicularis (Crab eating | 22 . . . 283 | 125/264 (47%) | |
| | macaque) (Cynomolgus monkey), | | | |
| | 473 aa. | | | |
| Q9VZ84 | CG7509 PROTEIN - Drosophila | 45 . . . 233 | 69/230 (30%) | 1e−18 |
| | melanogaster (Fruit fly), 633 aa. | 313 . . . 540 | 103/230 (44%) | |
| Q9BZR6 | NOGO RECEPTOR - Homo | 3 . . . 237 | 79/264 (29%) | 3e−18 |
| | sapiens (Human), 473 aa. | 22 . . . 283 | 121/264 (44%) | |

PFam analysis predicts that the NOV26a protein contains the domains shown in the Table 26F.

TABLE 26F
Domain Analysis of NOV26a

| | NOV26a | Identities/Similarities | Expect |
|---|---|---|---|
| Pfam Domain | Match Region | for the Matched Region | Value |
| LRR | 58 . . . 81 | 7/25 (28%) | 0.3 |
| | | 19/25 (76%) | |
| LRR | 108 . . . 131 | 10/25 (40%) | 0.11 |
| | | 19/25 (76%) | |
| LRR | 132 . . . 155 | 8/25 (32%) | 0.7 |
| | | 18/25 (72%) | |
| LRR | 158 . . . 181 | 11/25 (44%) | 0.00021 |
| | | 19/25 (76%) | |
| LRR | 182 . . . 204 | 10/25 (40%) | 0.093 |
| | | 18/25 (72%) | |
| LRRCT | 214 . . . 270 | 15/63 (24%) | 0.046 |
| | | 42/63 (67%) | |

### Example 27

The NOV27 clone was analyzed, and the nucleotide and encoded polypeptide sequences are shown in Table 27A.

TABLE 27A
NOV27 Sequence Analysis

| | |
|---|---|
| SEQ ID NO: 73 | 2358 bp |

| | |
|---|---|
| NOV27a, | GACTTCCTGGCTCGCCAGCCCCTTCCTTCCGGAGCCTGACCCGGGCCCGGGCGACCTC |
| CG106942-01 | |
| DNA Sequence | CCCGCGCGCTTCCCGGCCGCTGCCCAGGGGGTAGAGCGGGCGCAGCCGATCACTACCT |
| | GACGGCCTTTTTGGCGGCCTGGCCGGGCTGTGCAGGGTGGTAGGGCAAGACGCGCGGC |
| | TCCCAATTCTCCCCGGCGCCTTCGCCGGCCCCGGGCTTCTCGCGCTCCGCTCCGGGCT |
| | GCACCGAGTTGGGCCGGCGCGCCGCGTTGGTGTTGCCGCGCGGCGGCAGCTCAGAGTC |
| | TCCAGGTTGGGGCGGGCCTGGGCCGCACGGCTCCTCCACCCAGGTGACGCTGAGCAGG |
| | CTCAGGGTGAAGCCCAGGGAGATGCCCACGGCCACGGGCCCTGCGGGCCGCAGCACCG |
| | ACAGCAGCAGCGATGCCCGCATGGCGCCCGGACCGCGGGTCCCCGGCCCCGGCGAACC |
| | CCCAGAGCAGCCAGAGGAGTCTCCGAGGGGGCGGGACCGGGGAGGGGGCGGATCCGGA |
| | GGGCTCGGGCCCCGCGGGCGGGCCCGCTCCCTCCCCGCAGAGCAGAGCCAGCGGCCCG |
| | AGCCGAATCCCCGGAGCCGCGCCTCGATTCCCCTCCAGCAGCTGCTCTGGGCTGCGCA |
| | GGGTTCTTGCGCTCGGCACTGGAGCCTCAGCCGCGGCCGCAGCTGTCCGACGTGTCAC |
| | TGCAAGGGCCCCGCCCCCGGGGTGGGGTCTCGGGCTCTCGCTACCGGAGAGGGAGGAG |
| | AAGGGGGAGGTTAAAGGGGAAGGACCCCCGGAAGTGCCCCCTCCTCAGTGCGGGAGAG |
| | GGAGACGCCGGGGGCGGAGTCCCCTGCCTCCCGCGGCGTGGTTGGTGCGTCCCATGTG |
| | ACGTCAGAAGCAGCCCGCCCCTGCCTGGATGGTGCGCCCTGAGTGACGTCAGGAGCAG |
| | AGGCCGGAGCTGTCCATCAGCACCAAAGGCCGCGGGCGGGCTCAGGGCATGGGGCCGC |
| | GGTTCTGGGGCGGCCCGAGCCCCGGCTCCTGCGCCTTCCCCTTCCTCAGGCCCAGCCC |
| | GAGTTCCCGGACGCCGCGGGACTGGAGTGCCAGCCGGTGTTGGACGTGGAGCGGCGCC |
| | GCCACCGCGCCGACACCATTCTCTCCGGCCCAGCAGCCCCCTTCCTCGCACGACGGAC |
| | TTTCCCTGGACCCCAGCACTATGCCGGGGACTGTGGCAACACTGCGGTTCCAGCTGCT |
| | GCCCCCTGAGCCAGATGATGCCTTCTGGGGTGCACCTTGTGAACAGCCCCTGGAGCGC |
| | AGGTACCAGGCACTGCCGGCCCTCGTCTGCATCATGTGCTGTTTGTTTGGAGTCGTCT |
| | ACTGCTTCTTCGGTTACCGCTGCTTCAAGGCAGTGCTCTTTCTCACTGGGTTGCTGTT |
| | TGGCTCGGTGGTCATCTTCCTCCTCTGCTACCGAGAGCGGGTGCTAGAGACACAGCTG |
| | AGTGCTGGGGCGAGCGCGGGCATCGCTCTGGGCATCGGGCTGCTCTGCGGGCTGGTGG |
| | CCATGCTAGTGCGCAGCGTGGGCCTCTTCCTGGTGGGGCTGCTGCTCGGCCTGCTGCT |
| | CGCAGCTGCTGCCCTGCTGGGCTCCGCACCCTACTACCAGCCAGGCTCCGTGTGGGGT |
| | CCACTGGGGCTGTTGCTGGGGGGCGGCCTGCTCTGTGCCCTGCTCACTCTGCGCTGGC |
| | CCCGCCCACTCACCACCCTGGCCACCGCCGTGACTGGTGCTGCGCTGATCGCCACTGC |
| | CGCTGACTACTTCGCCGAGCTGCTACTGCTGGGGCGCTACGTGGTGGAGCGACTCCGG |
| | GCTGCTCCTGTGCCCCCACTCTGCTGGCGAAGCTGGGCCCTGCTGGCACTCTGGCCCC |
| | TGCTCAGCCTGATGGGCGTTCTGGTGCAGTGGAGGGTGACAGCTGAGGGGGACTCCCA |
| | CACGGAAGTGGTCATCAGCCGGCAGCGCCGACGCGTGCAACTGATGCGGATTCGGCAG |
| | CAGGAAGATCGCAAGGAGAAAAGGCGGAAAAAGAGACCTCCTCGGGCTCCCCTCAGAG |
| | GTCCCCGGGCTCCTCCCAGGCCTGGGCCACCAGATCCTGCTTATCGGCGCAGGCCAGT |
| | GCCCATCAAACGCTTCAATGGAGACGTCCTCTCCCCGAGCTATATCCAGAGCTTCCGA |
| | GACCGGCAGACCGGGAGCTCCCTGAGCTCCTTCATGGCCTCACCCACAGATGCGGACT |
| | ATGAGTATGGGTCCCGGGGACCTCTGACAGCCTGCTCAGGCCCCCCAGTGCGGGTATA |
| | GCCATATCTGTCTGTCTAGACTCTGCAGTCACCAGCTCTGACAGCTCGAGGAGGCCGG |
| | TAGGCTGCAATCAGCTTCCGGTTTGGTGGTCCTTCCCA |

| | | |
|---|---|---|
| ORF Start: ATG at 977 | | ORF Stop: TAG at 2261 |
| SEQ ID NO: 74 | 428 aa | MW at 46672.9 kD |

| | |
|---|---|
| NOV27a, | MGPRFWGGPSPGSCAFPFLRPSPSSRTPRDWSASRCWTWSGAATAPTPFSPAQQPPSS |
| CG106942-01 | |
| Protein Sequence | HDGLSLDPSTMPGTVATLRFQLLPPEPDDAFWGAPCEQPLERRYQALPALVCIMCCLF |
| | GVVYCFFGYRCFKAVLFLTGLLFGSVVIFLLCYRERVLETQLSAGASAGIALGIGLLC |
| | GLVAMLVRSVGLFLVGLLLGLLLAAAALLGSAPYYQPGSVWGPLGLLLGGGLLCALLT |
| | LRWPRPLTTLATAVTGAALIATAADYFAELLLLGRYVVERLRAAPVPPLCWRSWALLA |
| | LWPLLSLMGVLVQWRVTAEGDSHTEVVISRQRRRVQLMRIRQQEDRKEKRRKKRPPRA |
| | PLRGPRAPPRPGPPDPAYRRRPVPIKRFNGDVLSPSYIQSFRDRQTGSSLSSFMASPT |
| | DADYEYGSRGPLTACSGPPVRV |

Further analysis of the NOV27a protein yielded the following properties shown in Table 27B.

TABLE 27B
Protein Sequence Properties NOV27a

| | |
|---|---|
| PSort | 0.6000 probability located in plasma membrane; 0.4000 |
| analysis: | probability located in Golgi body; 0.3000 probability located |
| | in endoplasmic reticulum (membrane); 0.2400 probability |
| | located in nucleus |
| SignalP | No Known Signal Sequence Predicted |
| analysis: | |

A search of the NOV27a protein against the Geneseq database, a proprietary database that contains sequences published in patents and patent publication, yielded several homologous proteins shown in Table 27C.

TABLE 27C
Geneseq Results for NOV27a

| | | | Identities/ | |
|---|---|---|---|---|
| | | NOV27a | Similarities | |
| | | Residues/ | for the | |
| Geneseq | Protein/Organism/Length [Patent | Match | Matched | Expect |
| Identifier | #, Date] | Residues | Region | Value |
| AAM34044 | Peptide #8081 encoded by probe for | 79 . . . 188 | 60/111 (54%) | 2e−25 |
| | measuring placental gene expression - | 19 . . . 124 | 76/111 (68%) | |
| | Homo sapiens, 124 aa. | | | |
| | [WO200157272-A2, 09-AUG-2001] | | | |
| AAM20130 | Peptide #6564 encoded by probe for | 79 . . . 188 | 60/111 (54%) | 2e−25 |
| | measuring cervical gene expression - | 19 . . . 124 | 76/111 (68%) | |
| | Homo sapiens, 124 aa. | | | |
| | [WO200157278-A2, 09-AUG-2001] | | | |
| AAM73861 | Human bone marrow expressed | 79 . . . 188 | 60/111 (54%) | 2e−25 |
| | probe encoded protein SEQ ID NO: | 19 . . . 124 | 76/111 (68%) | |
| | 34167 - Homo sapiens, 124 aa. | | | |
| | [WO200157276-A2, 09-AUG-2001] | | | |
| AAM61147 | Human brain expressed single exon | 79 . . . 188 | 60/111 (54%) | 2e−25 |
| | probe encoded protein SEQ ID NO: | 19 . . . 124 | 76/111 (68%) | |
| | 33252 - Homo sapiens, 124 aa. | | | |
| | [WO200157275-A2, 09-AUG-2001] | | | |
| ABB24734 | Protein #6733 encoded by probe for | 79 . . . 188 | 60/111 (54%) | 2e−25 |
| | measuring heart cell gene expression - | 19 . . . 124 | 76/111 (68%) | |
| | Homo sapiens, 124 aa. | | | |
| | [WO200157274-A2, 09-AUG-2001] | | | |

In a BLAST search of public sequence databases, the NOV27a protein was found to have homology to the proteins shown in the BLASTP data in Table 27D.

TABLE 27D
Public BLASTP Results for NOV27a

| | | NOV27a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9VWD2 | CG14234 PROTEIN - Drosophila | 103 . . . 392 | 82/299 (27%) | 3e−20 |
| | melanogaster (Fruit fly), 381 aa. | 43 . . . 329 | 143/299 (47%) | |
| Q9CRG1 | 2010003B14RIK PROTEIN - Mus | 112 . . . 305 | 53/208 (25%) | 6e−07 |
| | musculus (Mouse), 556 aa | 286 . . . 490 | 95/208 (45%) | |
| | (fragment). | | | |
| Q9NS93 | SEVEN TRANSMEMBRANE | 115 . . . 305 | 50/205 (24%) | 4e−05 |
| | PROTEIN TM7SF3 - Homo | 303 . . . 504 | 88/205 (42%) | |
| | sapiens (Human), 570 aa. | | | |
| Q9NUS4 | CDNA FLJ11169 FIS, CLONE | 115 . . . 305 | 50/205 (24%) | 4e−05 |
| | PLACE1007282 - Homo sapiens | 303 . . . 504 | 88/205 (42%) | |
| | (Human), 570 aa. | | | |
| O28838 | Hypothetical protein AF1434 - | 107 . . . 304 | 51/201 (25%) | 7e−04 |
| | Archaeoglobus fulgidus, 199 aa. | 14 . . . 188 | 82/201 (40%) | |

### Example 28

The NOV28 clone was analyzed, and the nucleotide and encoded polypeptide sequences are shown in Table 28A.

TABLE 28A
NOV28 Sequence Analysis

| | |
|---|---|
| SEQ ID NO: 75 | 2177 bp |

| | |
|---|---|
| NOV28a, | ATTCCCCTTGCCGACCCACATACACCATGAAGAGGTGCAGATCGGACGAGCTGCAGCA |
| CG107513-01 | |
| DNA Sequence | ACAACAGGGCGAGGAGGATGGAGCTGGGCTGGAAGATGCCGCTTCCCACCTGCCGGGC |
| | GCGGACCTCCGGCCTGGGGAGACCACGGGTGCTAACTCTGCTGGCGGGCCAACTTCAG |
| | ACGCCGGCGCTGCCGCGGCGCCCAACCCAGGTCCCCGAAGCAAGCCTCCTGATTTAAA |
| | GAAAATCCAGCAGCTGTCAGAGGGCTCCATGTTTGGCCACGGTCTGAAGCACCTGTTC |
| | CACAGCCGCCGTCGGTCTCGGGAAAGGGAGCACCAGACGTCTCAGGATTCCCAGCAGC |
| | ATCAGCAGCAGCAGGGTATGTCCGACCATGACTCCCCAGATGAGAAGGAGCGCTCTCC |
| | GGAGATGCATCGCGTCTCCTACGCCATGTCCCTGCACGACCTGCCCGCCCGGCCCACC |
| | GCCTTCAACCGCGTGCTGCAGCAGATCCGCTCCCGGCCCTCCATCAAGCGGGGCGCCA |
| | GCCTGCACAGCAGCAGTGGGGGCGGCAGCAGCGGGAGCAGCAGCCGGCGCACCAAGAG |
| | TAGCTCCCTGGAGCCCCAGCGTGGCAGCCCTCACCTGCTGCGCAAGGCCCCCCAGGAC |
| | AGCAGCCTGGCCGCCATCCTGCACCAGCACCAGTGCCGTCCCCGCTCTTCCTCCACCA |
| | CCGACACTGCTCTGCTGCTGGCCGACGGCAGCAACGTGTACCTCCTGGCTGAGGAGGC |
| | CGAAGGCATCGGGGACAAGGTGGATAAGGGAGACCTGGTGGCCCTGAGCCTCCCCGCC |
| | GGCCATGGTGACACCGACGGCCCCATCAGCCTGGACGTGCCCGATGGGGCACCGGACC |
| | CCCAGCGGACCAAGGCCGCCATTGACCACCTGCACCAGAAGATCCTGAAGATCACCGA |
| | GCAGATCAAGATTGAGCAGGAGGCTCGCGACGACAATGTGGCAGAGTATCTGAAACTG |
| | GCCAACAACGCGGACAAGCAGCAGGTGTCACGCATCAAGCAAGTGTTCGAGAAGAAGA |
| | ACCAGAAGTCAGCCCAGACCATCGCCCAGCTGCACAAGAAGCTGGAGCACTACCGCCG |
| | GCGCCTGAAGGAGATTGAGCAGAACGGGCCCTCGCGGCAGCCCAAGGACGTGCTGCGG |
| | GACATGCAGCAGGGGCTGAAGGACGTGGGCGCCAACGTGCGCGCAGGCATCAGCGGCT |
| | TTGGGGGCGGCGTGGTGGAGGGCGTCAAGGGCAGCCTCTCTGGCCTCTCACAGGCCAC |
| | CCACACCGCCGTGGTGTCCAAGCCCCGGGAGTTTGCCAGCCTCATCCGCAACAAGTTT |
| | GGCAGTGCTGACAACATCGCCCACCTGAAGGACCCCCTGGAAGATGGGCCCCCTGAGG |
| | AGGCAGCCCGGGCACTGAGCGGCAGTGCCACACTCGTCTCCAGCCCCAAGTATGGCAG |
| | CGATGATGAGTGCTCCAGCGCCACGCTCAGCTCAGCCGGGGCAGGCAGCAACTCTGGG |
| | GCTGGGCCTGGTGGGGCGCTGGGGAGCCCTAAGTCCAATGCACTGTATGGTGCTCCTG |
| | GAAACCTGGATGCTCTGCTGGAAGAGCTACGGGAGATCAAGGAGGGACAGTCTCACCT |
| | GGAGGACTCCATGGAAGACCTGAAGACTCAGCTGCAGAGGGACTACACCTACATGACC |
| | CAGTGCCTGCAGGAGGAGCGCTACAGGTACGAGCGGCTGGAGGAGCAGCTCAACGACC |
| | TGACTGAGCTTCATCAGAACGAGATGACGAACCTGAAGCAGGAGCTGGCCAGCATGGA |
| | GGAGAAGGTGGCCTACCAGTCCTATGAGAGGGCACGGGACATCCAGGAGGCCGTGGAG |
| | TCCTGCCTGACCCGGGTCACCAAGCTGGAGCTGCAGCAGCAACAGCAGCAGGTGGTAC |
| | AGCTGGAGGGCGTGGAGAATGCCAACGCGCGGGCGCTGCTGGGCAAGTTCATCAACGT |
| | GATCCTGGCGCTCATGGCCGTGCTGCTGGTGTTCGTGTCCACCATCGCCAACTTCATC |
| | ACGCCCCTCATGAAGACACGCCTGCGCATCACCAGCACCACCCTCCTGGTCCTCGTCC |
| | TGTTCCTCCTCTGGAAGCACTGGGACTCCCTCACCTACCTCCTGGAGCACGTGTTGCT |
| | GCCCAGCTGAGTGGCCAGCCACACCAACCCT |

| | | |
|---|---|---|
| ORF Start: ATG at 27 | | ORF Stop: TGA at 2154 |
| SEQ ID NO:76 | 709 aa | MW at 77503.9 kD |

| | |
|---|---|
| NOV28a, | MKRCRSDELQQQQGEEDGAGLEDAASHLPGADLRPGETTGANSAGGPTSDAGAAAAPN |
| CG107513-01 | |
| Protein Sequence | PGPRSKPPDLKKIQQLSEGSMFGHGLKHLFHSRRRSREREHQTSQDSQQHQQQQGMSD |
| | HDSPDEKERSPEMHRVSYAMSLHDLPARPTAFNRVLQQIRSRPSIKRGASLHSSSGGG |
| | SSGSSSRRTKSSSLEPQRGSPHLLRKAPQDSSLAAILHQHQCRPRSSSTTDTALLLAD |
| | GSNVYLLAEEAEGIGDKVDKGDLVALSLPAGHGDTDGPISLDVPDGAPDPQRTKAAID |
| | HLHQKILKITEQIKIEQEARDDNVAEYLKLANNADKQQVSRIKQVFEKKNQKSAQTIA |
| | QLHKKLEHYRRRLKEIEQNGPSRQPKDVLRDMQQGLKDVGANVRAGISGFGGGVVEGV |
| | KGSLSGLSQATHTAVVSKPREFASLIRNKFGSADNIAHLKDPLEDGPPEEAARALSGS |
| | ATLVSSPKYGSDDECSSATLSSAGAGSNSGAGPGGALGSPKSNALYGAPGNLDALLEE |
| | LREIKEGQSHLEDSMEDLKTQLQRDYTYMTQCLQEERYRYERLEEQLNDLTELHQNEM |
| | TNLKQELASMEEKVAYQSYERARDIQEAVESCLTRVTKLELQQQQQQVVQLEGVENAN |
| | SLTYLLEHVLLPS |

Further analysis of the NOV28a protein yielded the following properties shown in Table 28B.

TABLE 28B
Protein Sequence Properties NOV28a

| | |
|---|---|
| PSort | 0.6000 probability located in plasma membrane; 0.4000 probability located in |
| analysis: | Golgi body; 0.3000 probability located in endoplasmic reticulum (membrane); |
| | 0.3000 probability located in microbody (peroxisome) |
| SignalP | No Known Signal Sequence Predicted |
| analysis: | |

A search of the NOV28a protein against the Geneseq database, a proprietary database that contains sequences published in patents and patent publication, yielded several homologous proteins shown in Table 28C.

TABLE 28C
Geneseq Results for NOV28a

| | | NOV28a | Identities/ | |
|---|---|---|---|---|
| | | Residues/ | Similarities for | |
| Geneseq | Protein/Organism/Length [Patent | Match | the Matched | Expect |
| Identifier | #, Date] | Residues | Region | Value |
| AAY94907 | Human secreted protein clone | 57 . . . 703 | 359/672 (53%) | e−178 |
| | ca106_19x protein sequence SEQ | 13 . . . 646 | 438/672 (64%) | |
| | ID NO: 20 - Homo sapiens, 653 aa. | | | |
| | [WO200009552-A1, 24-FEB-2000] | | | |
| AAM78708 | Human protein SEQ ID NO 1370 - | 249 . . . 708 | 258/466 (55%) | e−134 |
| | Homo sapiens, 477 aa. | 26 . . . 476 | 326/466 (69%) | |
| | [WO200157190-A2, 09-AUG-2001] | | | |
| AAU28090 | Novel human secretory protein, Seq | 258 . . . 708 | 254/457 (55%) | e−132 |
| | ID No 259 - Homo sapiens, 446 aa. | 4 . . . 445 | 320/457 (69%) | |
| | [WO200166689-A2, 13-SEP-2001] | | | |
| AAM40705 | Human polypeptide SEQ ID NO | 352 . . . 703 | 224/355 (63%) | e−117 |
| | 5636 - Homo sapiens, 369 aa. | 13 . . . 362 | 267/355 (75%) | |
| | [WO200153312-A1, 26-JUL-2001] | | | |
| AAM38919 | Human polypeptide SEQ ID NO | 379 . . . 703 | 209/328 (63%) | e−108 |
| | 2064 - Homo sapiens, 331 aa. | 2 . . . 324 | 248/328 (74%) | |
| | [WO200153312-A1, 26-JUL-2001] | | | |

In a BLAST search of public sequence databases, the NOV28a protein was found to have homology to the proteins shown in the BLASTP data in Table 28D.

TABLE 28D
Public BLASTP Results for NOV28a

| | | NOV28a | Identities/ | |
|---|---|---|---|---|
| Protein | | Residues/ | Similarities for | |
| Accession | | Match | the Matched | Expect |
| Number | Protein/Organism/Length | Residues | Portion | Value |
| O75069 | Hypothetical protein KIAA0481 | 69 . . . 709 | 638/641 (99%) | 0.0 |
| | (Cerebral protein-11) (hucep-11) - | 10 . . . 650 | 640/641 (99%) | |
| | Homo sapiens (Human), 650 aa | | | |
| | (fragment). | | | |
| Q9ULS5 | Hypothetical protein KIAA1145 - | 250 . . . 708 | 258/465 (55%) | e−134 |
| | Homo sapiens (Human), 467 aa | 17 . . . 466 | 325/465 (69%) | |
| | (fragment). | | | |
| AAH26867 | SIMILAR TO KIAA1145 | 258 . . . 708 | 249/457 (54%) | e−129 |
| | PROTEIN - Mus musculus | 4 . . . 445 | 316/457 (68%) | |
| | (Mouse), 446 aa. | | | |
| O94876 | Hypothetical protein KIAA0779 - | 393 . . . 703 | 202/314 (64%) | e−105 |
| | Homo sapiens (Human), 320 aa | 1 . . . 313 | 241/314 (76%) | |
| | (fragment). | | | |
| Q9VI21 | CG1021 PROTEIN - Drosophila | 288 . . . 675 | 188/409 (45%) | 1e−84 |
| | melanogaster (Fruit fly), 638 aa. | 252 . . . 638 | 248/409 (59%) | |

### Example 29

The NOV29 clone was analyzed, and the nucleotide and encoded polypeptide sequences are shown in Table 29A.

TABLE 29A
NOV29 Sequence Analysis

| | |
|---|---|
| SEQ ID NO: 77 | 664 bp |

| | |
|---|---|
| NOV29a, | ATCGCCCCTCCTGCGCTAGCGGAGGTGATCGCCGCGGCGATGCCGGAGGAGGGTTCGG |
| CG107533-02 | |
| DNA Sequence | GCTGCTCGGTGCGGCGCAGGCCCTATGGGTGCGTCCTGCGGGCTGCTTTGGTCCCATT |
| | GGTCGCGGGCTTGGTGATCTGCCTCGTGGTGTGCATCCAGCGCTTCGCACAGGCTCAG |
| | CAGCAGCTGCCGCTCGAGTCACTTGGGGACCTCAGCAGGACCCCAGGCTATACTGGCA |
| | GGGGGGCCCAGCACTGGGCCGCTCCTTCCTGCATGGACCAGAGCTGGACAAGGGGCAG |
| | CTACGTATCCATCGTGATGGCATCTACATGGTACACATCCAGGTGACGCTGGCCATCT |
| | GCTCCTCCACGACGGCCTCCAGGCACCACCCCACCACCCTGGCCGTGGGAATCTGCTC |
| | TCCCGCCTCCCGTAGCATCAGCCTGCTGCGTCTCAGCTTCCACCAAGGTTGTACCATT |
| | GCCTCCCAGCGCCTGACGCCCCTGGCCCGAGGGGACACACTCTGCACCAACCTCACTG |
| | GGACACTTTTGCCTTCCCGAAACACTGATGAGACCTTCTTTGGAGTGCAGTGGGTGCG |
| | CCCCTGACCACTGCTGCTGATTAGGGTTTTTTAAATTTTATTTTATTTTATTTAAGTT |
| | CAAGAGAAAAAGTGTACACACAGGGG |

| | | |
|---|---|---|
| ORF Start: ATG at 40 | | ORF Stop: TGA at 334 |
| SEQ ID NO: 78 | 98 aa | MW at 10705.2kD |

| | |
|---|---|
| NOV29a, | MPEEGSGCSVRRRPYGCVLRAALVPLVAGLVICLVVCIQRFAQAQQQLPLESLGDLSR |
| CG107533-02 | |
| Protein Sequence | TPGYTGRGAQHWAAPSCMDQSWTRGSYVSIVMASTWYTSR |

Further analysis of the NOV29a protein yielded the following properties shown in Table 29B.

TABLE 29B
Protein Sequence Properties NOV29a

| | |
|---|---|
| PSort | 0.7900 probability located in plasma membrane; |
| analysis: | 0.3000 probability located in Golgi body; |
| | 0.2000 probability located in endoplasmic |
| | reticulum (membrane); |
| | 0.1000 probability located in mitochondrial inner membrane |
| SignalP | Cleavage site between residues 45 and 46 |
| analysis: | |

A search of the NOV29a protein against the Geneseq database, a proprietary database that contains sequences published in patents and patent publication, yielded several homologous proteins shown in Table 29C.

TABLE 29C
Geneseq Results for NOV29a

| | | NOV29a | Identities/ | |
|---|---|---|---|---|
| | | Residues/ | Similarities for | |
| Geneseq | Protein/Organism/Length [Patent | Match | the Matched | Expect |
| Identifier | #, Date] | Residues | Region | Value |
| AAR50121 | CD27L - Homo sapiens, 193 aa. | 1 . . . 54 | 54/54 (100%) | 7e−25 |
| | [WO9405691-A, 17-MAR-1994] | 1 . . . 54 | 54/54 (100%) | |
| AAW41180 | CD27 ligand - Homo sapiens, 216 | 39 . . . 54 | 16/16 (100%) | 0.16 |
| | aa. [US5716805-A, 10-FEB-1998] | 62 . . . 77 | 16/16 (100%) | |
| AAR50122 | sCD27L-3 - Homo sapiens, 216 aa. | 39 . . . 54 | 16/16 (100%) | 0.16 |
| | [WO9405691-A, 17-MAR-1994] | 62 . . . 77 | 16/16 (100%) | |
| AAR53971 | CD27-L type II transmembrane | 39 . . . 54 | 16/16 (100%) | 0.16 |
| | protein - Mammalia, 216 aa. | 62 . . . 77 | 16/16 (100%) | |
| | [WO9410308-A, 11-MAY-1994] | | | |
| AAG26041 | Zea mays protein fragment SEQ ID | 24 . . . 72 | 19/55 (34%) | 2.4 |
| | NO: 30347 - Zea mays subsp. mays, | 6 . . . 59 | 26/55 (46%) | |
| | 172 aa. [EP1033405-A2, 06-SEP-2000] | | | |

In a BLAST search of public sequence databases, the NOV29a protein was found to have homology to the proteins shown in the BLASTP data in Table 29D.

TABLE 29D
Public BLASTP Results for NOV29a

| | | NOV29a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q96J57 | TUMOR NECROSIS FACTOR | 1 . . . 54 | 54/54 (100%) | 2e−24 |
| | (LIGAND) SUPERFAMILY, | 1 . . . 54 | 54/54 (100%) | |
| | MEMBER 7 - Homo sapiens | | | |
| | (Human), 193 aa. | | | |
| P32970 | CD27 ligand (CD27-L) (CD70 | 1 . . . 54 | 54/54 (100%) | 2e−24 |
| | antigen) - Homo sapiens (Human), | 1 . . . 54 | 54/54 (100%) | |
| | 193 aa. | | | |
| Q9KFY7 | HYPOTHETICAL PROTEIN | 39 . . . 98 | 18/61 (29%) | 7.4 |
| | BH0329 - Bacillus halodurans, 423 aa. | 240 . . . 300 | 28/61 (45%) | |
| Q9RC64 | UNKNOWN - Bacillus halodurans, | 39 . . . 98 | 18/61 (29%) | 7.4 |
| | 262 aa. | 79 . . . 139 | 28/61 (45%) | |
| O34255 | PURL PROTEIN - Wolinella | 70 . . . 93 | 11/24 (45%) | 9.7 |
| | succinogenes, 331 aa (fragment). | 8 . . . 31 | 15/24 (61%) | |

### Example 30

The NOV30 clone was analyzed, and the nucleotide and encoded polypeptide sequences are shown in Table 30A.

TABLE 30A
NOV30 Sequence Analysis

| | |
|---|---|
| SEQ ID NO: 79 | 2840 bp |

| | |
|---|---|
| NOV30a, | CGGCAGTGGCAGGAGCCGCCTTTCCGATTCCCTACGATGCGGGTGCTGAGCTATGGCA |
| CG107562-01 | |
| DNA Sequence | AAGGGCAGCGAAGTGACGAGCGAGACCCGCGTACGACTGTGAAAGCCACCTGGAGCCA |
| | CCTTGCCGGGATTGTACCTGCAGGCAGAAAGTCTTCCTACGACCGTCTTTTCCCTTAG |
| | AGGCACCAGAATCCCTGTAACCATTCATCCAGGTGTTGAGAAGATATGTAGCAGCCGA |
| | GCACCCATCTTTTGACACCGTCCTCTGAAATCAGCTTTGGAGATGCTTTCACTCTGTC |
| | CGTCTTCTGCAGCAGCCAGGCAGAGTGCCGACTCCTTCACAGCCGTGAGGAACTCTTC |
| | AGGCTCCAGAAGCTCTTAAACCTGATCTACAATGGAAAAAATTCTTTTTTATCTGTTT |
| | CTCATTGGCATAGCAGTGAAAGCTCAGATCTGTCCAAAGCGTTGTGTCTGTCAGATTT |
| | TGTCTCCTAATCTTGCAACCCTTTGTGCCAAGAAAGGGCTTTTATTTGTTCCACCAAA |
| | CATTGACAGAAGAACTGTGGAACTGCGGTTGGCAGACAATTTTGTTACAAATATTAAA |
| | AGGAAAGATTTTGCCAATATGACCAGCTTGGTGGACCTGACTCTATCCAGGAATACAA |
| | TAAGTTTTATTACACCTCATGCTTTCGCTGACCTACGAAATTTGAGGGCTTTGCATTT |
| | GAATAGCAACAGATTGACTAAAATTACAAATGATATGTTCAGTGGTCTTTCCAATCTT |
| | CATCATTTGATACTGAACAACAATCAGCTGACTTTAATTTCCTCTACAGCGTTTGATG |
| | ATGTCTTCGCCCTTGAGGAGCTGGATCTGTCCTATAATAATCTAGAAACCATTCCTTG |
| | GGATGCTGTTGAGAAGATGGTTAGCTTGCATACCCTTAGTTTGGATCACAATATGATT |
| | GATAACATTCCTAAGGGGACCTTCTCCCATTTGCACAAGATGACTCGGTTAGATGTGA |
| | CATCAAATAAATTGCAGAAGCTACCACCTGACCCTCTCTTTCAGCGAGCTCAGGTACT |
| | AGCAACCTCAGGAATCATAAGCCCATCTACTTTTGCATTAAGTTTTGGTGGAAACCCC |
| | TTGCATTGCAATTGTGAATTGTTGTGGTTGAGGCGTCTGTCCAGAGAAGATGACTTAG |
| | AGACCTGTGCTTCTCCTCCACTTTTAACTGGCCGCTACTTTTGGTCAATTCCTGAAGA |
| | AGAGTTTTTGTGTGAGCCTCCTCTCATTACTCGTCATACACATGAGATGAGAGTCCTG |
| | GAGGGACAAAGGGCAACACTGAGGTGCAAAGCCAGGGGAGACCCTGAGCCTGCAATTC |
| | ACTGGATTTCTCCTGAAGGGAAGCTTATTTCAAATGCAACAAGATCTCTGGTGTATGA |
| | TAACGGAACACTTGACATTCTTATCACAACTGTAAAGGATACAGGTGCTTTTACCTGC |
| | ATTGCTTCCAATCCTGCTGGGGAAGCAACACAAATAGTGGATCTTCATATAATTAAGC |
| | TCCCTCACTTACTAAATAGTACAAACCATATCCATGAGCCTGATCCTGGTTCTTCAGA |
| | TATCTCAACTTCTACCAAGTCAGGTTCTAATACAAGCAGTAGTAATGGTGATACTAAA |
| | TTGAGTCAAGATAAAATTGTGGTGGCAGAAGCTACATCATCAACGGCACTACTTAAAT |
| | TTAATTTTCAAAGAAATATCCCTGGAATACGTATGTTTCAAATCCAGTACAATGGTAC |
| | TTATGATGACACCCTTGTTTACAGGATGATACCTCCTACGAGCAAAACTTTTCTGGTC |
| | AATAATCTGGCTGCTGGAACTATGTATGACTTGTGTGTCTTGGCCATATATGATGATG |
| | GCATCACTTCCCTCACTGCCACAAGAGTCGTGGGTTGCATCCAGTTTACTACGGAACA |
| | GGATTATGTGCGTTGCCATTTCATGCAGTCCCAGTTTTTGGGAGGCACCATGATTATT |
| | ATTATTGGTGGAATCATTGTAGCATCTGTGCTGGTATTCATCATTATTCTGATGATCC |
| | GGTATAAGGTTTGCAACAATAATGGGCAACACAAGGTCACCAAGGTTAGCAATGTTTA |
| | TTCCCAAACTAACGGGGCTCAAATACAAGGCTGTAGTGTAACGCTGCCCCAGTCCGTG |
| | TCCAAACAAGCTGTGGGACACGAAGAGAATGCCCAGTGTTGTAAAGCTACCAGTGACA |
| | ATGTGATTCAATCTTCAGAAACTTGTTCGAGTCAAGACTCCTCTACCACTACCTCTGC |
| | TTTGCCTCCTTCCTGGACTTCAAGCACTTCTGTGTCCCAAAAGCAGAAAAGAAAGACT |
| | GGCACAAAGCCAAGTACAGAACCACAGAATGAAGCCGTCACAAATGTTGAATCCCAAA |
| | ACACTAACAGGAACAACTCAACTGCCTTGCAGTTAGCTAGCCGTCCTCCCGATTCTGT |
| | CACAGAGGGGCCCACGTCTAAAAGAGCACATATAAAGCCAAGTAAGTTTATCACTTTG |
| | CCTGCTGAGAGATCCGGAGCAAGGCACAAGTACTCCCTCAATGGAGAATTAAAGGAAT |
| | ACTATTGTTATATTAACTCGCCGAACACATGTGGACTGTTTCCTAAAAGAAGCATGTC |
| | TATGAATGTGATGTTTATTCAGTCTGACTGTTCTGATGGTCATAGTGGAAAGGCAACT |
| | CTCAAATTCTGAGGGACTACTGGAAAGCTCTGTGTAATTTATAATTTCTTTTTCATGA |
| | AAAATCATTTTGAGAACTCACATAGAAGATTGGAATTTGCAATTCCAATGCTGTGTAT |
| | AAATCAACCTTCTCAGATGCTTTGCTGACTAATGTTGACCAGATTGTCCAGGAAAC |

| | | |
|---|---|---|
| ORF Start: ATG at 380 | | ORF Stop: TGA at 2678 |
| SEQ ID NO: 80 | 766 aa | MW at 84690.6 kD |

| | |
|---|---|
| NOV30a, | MEKILFYLFLIGIAVKAQICPKRCVCQILSPNLATLCAKKGLLFVPPNIDRRTVELRL |
| CG107562-01 | |
| Protein Sequence | ADNFVTNIKRKDFANMTSLVDLTLSRNTISFITPHAFADLRNLRALHLNSNRLTKITN |
| | DMFSGLSNLHHLILNNNQLTLISSTAFDDVFALEELDLSYNNLETIPWDAVEKMVSLH |
| | TLSLDHNMIDNIPKGTFSHLHKMTRLDVTSNKLQKLPPDPLFQRAQVLATSGIISPST |
| | FALSFGGNPLHCNCELLWLRRLSREDDLETCASPPLLTGRYFWSIPEEEFLCEPPLIT |
| | RHTHEMRVLEGQRATLRCKARGDPEPAIHWISPEGKLISNATRSLVYDNGTLDILITT |
| | VKDTGAFTCIASNPAGEATQIVDLHIIKLPHLLNSTNHIHEPDPGSSDISTSTKSGSN |
| | TSSSNGDTKLSQDKIVVAEATSSTALLKFNFQRNIPGIRMFQIQYNGTYDDTLVYRMI |
| | PPTSKTFLVNNLAAGTMYDLCVLAIYDDGITSLTATRVVGCIQFTTEQDYVRCHFMQS |
| | QFLGGTMIIIIGGIIVASVLVFIIILMIRYKVCNNNGQHKVTKVSNVYSQTNGAQIQG |
| | CSVTLPQSVSKQAVGHEENAQCCKATSDNVIQSSETCSSQDSSTTTSALPPSWTSSTS |
| | VSQKQKRKTGTKPSTEPQNEAVTNVESQNTNRNNSTALQLASRPPDSVTEGPTSKPAH |
| | IKPSKFITLPAERSGARHKYSLNGELKEYYCYINSPNTCGLFPKRSMSMNVMFIQSDC |
| | SDGHSGKATLKF |

| | |
|---|---|
| SEQ ID NO: 81 | 2388 bp |

| | |
|---|---|
| NOV30b, | GCTCTTAAACCTGATCTACAATGGAAAAAATTCTTTTTTATCTGTTTCTCATTGGCAT |
| CG107562-02 | |
| DNA Sequence | AGCAGTGAAAGCTCAGATCTGTCCAAAGCGTTGTGTCTGTCAGATTTTGTCTCCTAAT |
| | CTTGCAACCCTTTGTGCCAAGAAAGGGCTTTTATTTGTTCCACCAAACATTGACAGAA |
| | GAACTGTGGAACTGCGGTTGGCAGACAATTTTGTTACAAATATTAAAAGGAAAGATTT |
| | TGCCAATATGACCAGCTTGGTGGACCTGACTCTATCCAGGAATACAATAAGTTTTATT |
| | ACACCTCATGCTTTCGCTGACCTACGAAATTTGAGGGCTTTGCATTTGAATAGCAACA |
| | GATTGACTAAAATTACAAATGATATGTTCAGTGGTCTTTCCAATCTTCATCATTTGAT |
| | ACTGAACAACAATCAGCTGACTTTAATTTCCTCTACAGCGTTTGATGATGTCTTCGCC |
| | CTTGAGGAGCTGGATCTGTCCTATAATAATCTAGAAACCATTCCTTGGGATGCTGTTG |
| | AGAAGATGGTTAGCTTGCATACCCTTAGTTTGGATCACAATATGATTGATAACATTCC |
| | TAAGGGGACCTTCTCCCATTTGCACAAGATGACTCGGTTAGATGTGACATCAAATAAA |
| | TTGCAGAAGCTACCACCTGACCCTCTCTTTCAGCGAGCTCAGGTACTAGCAACCTCAG |
| | GAATCATAAGCCCATCTACTTTTGCATTAAGTTTTGGTGGAAACCCCTTGCATTGCAA |
| | TTGTGAATTGTTGTGGTTGAGGCGTCTGTCCAGAGAAGATGACTTAGAGACCTGTGCT |
| | TCTCCTCCACTTTTAACTGGCCGCTACTTTTGGTCAATTCCTGAAGAAGAGTTTTTGT |
| | GTGAGCCTCCTCTCATTACTCGTCATACACATGAGATGAGAGTCCTGGAGGGACAAAG |
| | GGCAACACTGAGGTGCAAAGCCAGGGGAGACCCTGAGCCTGCAATTCACTGGATTTCT |
| | CCTGAAGGGAAGCTTATTTCAAATGCAACAAGATCTCTGGTGTATGATAACGGAACAC |
| | TTGACATTCTTATCACAACTGTAAAGGATACAGGTGCTTTTACCTGCATTGCTTCCAA |
| | TCCTGCTGGGGAAGCAACACAAATAGTGGATCTTCATATAATTAAGCTCCCTCACTTA |
| | CTAAATAGTACAAACCATATCCATGAGCCTGATCCTGGTTCTTCAGATATCTCAACTT |
| | CTACCAAGTCAGGTTCTAATACAAGCAGTAGTAATGGTGATACTAAATTGAGTCAAGA |
| | TAAAATTGTGGTGGCAGAAGCTACATCATCAACGGCACTACTTAACTTTACTTTTCAA |
| | AGAACTATCCCTGGAATACGTATGTTTCAAATCCAGTACAATGGTACTTATGATGACA |
| | CCCTTGTTTACAGGATGATACCTCCTACGAGCAAAACTTTTCTGGTCAATAATCTGGC |
| | TGCTGGAACTATGTATGACTTGTGTGTCTTGGCCATATATGATGATGGCATCACTTCC |
| | CTCACTGCCACAAGAGTCGTGGGTTGCATCCAGTTTACTACGGAACAGGATTATGTGC |
| | GTTGCCATTTCATGCAGTCCCAGTTTTTGGGAGGCACCATGATTATTATTATTGGTGG |
| | AATCATTGTAGCATCTGTGCTGGTATTCATCATTATTCTGATGATCCGGTATAAGGTT |
| | TGCAACAATAATGGGCAACACAAGGTCACCAAGGTTAGCAATGTTTATTCCCAAACTA |
| | ACGGGGCTCAAATACAAGGCTGTAGTGTAACGCTGCCCCAGTCCGTGTCCAAACAAGC |
| | TGTGGGACACGAAGAGATTGCCCAGTGTTGTAAAGCTACCAGTGACAATGTGATTCAA |
| | TCTTCAGAAACTTGTTCGAGTCAGGACTCCTCTACCACTACCTCTGCTTTGCCTCCTT |
| | CCTGGACTTCAAGCACTTCTGTGTCCCAAAAGCAGAAAAGAAAGACTGGCACAAAGCC |
| | AAGTACAGAACCACAGAATGAAGCCGTCACAAATGTTGAATCCCAAAACACTAACAGG |
| | AACAACTCAACTGCCTTGCAGTTAGCTAGCCGTCCTCCCGATTCTGTCACAGAGGGGC |
| | CCACGTCTAAAAGAGCACATATAAAGCCAAATGCTTTGCTGACTAATGTTGACCAGAT |
| | TGTCCAGGAAACACAGAGGCTGGAGTTAATCTGAAGAGCACCACTTCTCCTCTCTCTC |
| | CTGAAAAAATTTGCCACTGATATTTTTACTGGATAAAATTCAAAAATGTTTCAATTCA |
| | CAAAGGCTAATTGTTGAACTGGTGTCGTAGAAGAAATTGTCTACAGGAGCCAAGGTGA |
| | AAGTCTCTGATGACGGCGGAACTGGCTCCATTAGACCATGGTTCATCCTCTTTTAAAA |
| | ACAAATTTTT |

| | | |
|---|---|---|
| ORF Start: ATG at 21 | | ORF Stop: TGA at 2178 |
| SEQ ID NO: 82 | 719 aa | MW at 79402.7 kD |

| | |
|---|---|
| NOV30b, | MEKILFYLFLIGIAVKAQICPKRCVCQILSPNLATLCAKKGLLFVPPNIDRRTVELRL |
| CG107562-02 | |
| Protein Sequence | ADNFVTNIKRKDFANMTSLVDLTLSRNTISFITPHAFADLPNLRALHLNSNRLTKITN |
| | DMFSGLSNLHHLILNNNQLTLISSTAFDDVFALEELDLSYNNLETIPWDAVEKMVSLH |
| | TLSLDHNMIDNIPKGTFSHLHKMTRLDVTSNKLQKLPPDPLFQRAQVLATSGIISPST |
| | FALSFGGNPLHCNCELLWLRRLSREDDLETCASPPLLTGRYFWSIPEEEFLCEPPLIT |
| | RHTHEMRVLEGQRATLRCKARGDPEPAIHWISPEGKLISNATRSLVYDNGTLDILITT |
| | VKDTGAFTCIASNPAGEATQIVDLHIIKLPHLLNSTNHIHEPDPGSSDISTSTKSGSN |
| | TSSSNGDTKLSQDKIVVAEATSSTALLNFTFQRTIPGIRMFQIQYNGTYDDTLVYRMI |
| | PPTSKTFLVNNLAAGTMYDLCVLAIYDDGITSLTATRVVGCIQFTTEQDYVRCHFMQS |
| | QFLGGTMIIIIGGIIVASVLVFIIILMIRYKVCNNNGQHKVTKVSNVYSQTNGAQIQG |
| | CSVTLPQSVSKQAVGHEEIAQCCKATSDNVIQSSETCSSQDSSTTTSALPPSWTSSTS |
| | VSQKQKRKTGTKPSTEPQNEAVTNVESQNTNRNNSTALQLASRPPDSVTEGPTSKRAH |
| | IKPNALLTNVDQIVQETQRLELI |

| | |
|---|---|
| SEQ ID NO: 83 | 1545 bp |

| | |
|---|---|
| NOV30c, | GGATCCCAGATCTGTCCAAAGCGTTGTGTCTGTCAGATTTTGTCTCCTAATCTTGCAA |
| 210086373 | |
| DNA Sequence | CCCTTTGTGCCAAGAAAGGGCTTTTATTTGTTCCACCAAACATTGACAGAAGAACTGT |
| | GGAACTGCGGTTGGCAGACAATTTTGTTACAAATATTAAAAGGAAAGATTTTGCCAAT |
| | ATGAGCAGCTTGGTGGACCTGACTCTATCCAGGAATACAATAAGTTTTATTACACCTC |
| | ATGCTTTCGCTGACCTACGAAATTTGAGGGCTTTGCATTTGAATAGCAACAGATTGAC |
| | TAAAATTACAAATGATATGTTCAGTGGTCTTTCCAATCTTCATCATTTGATACTGAAC |
| | AACAATCAGCTGACTTTAATTTCCTCTACAGCGTTTGATGATGTCTTCGCCCTTGAGG |
| | AGCTGGATCTGTCCTATAATAATCTAGAAACCATTCCTTGGGATGCTGTTGAGAAGAT |
| | GGTTAGCTTGCATACCCTTAGTTTGGATCACAATATGATTGATAACATTCCTAAGGGG |
| | ACCTTCTCCCATTTGCACAAGATGACTCGGTTAGATGTGACATCAAATAAATTGCAGA |
| | AGCTACCACCTGACCCTCTCTTTCAGCGAGCTCAGGTACTAGCAACCTCAGGAATCAT |
| | AAGCCCATCTACTTTTGCATTAAGTTTTGGTGGAAACCCCTTGCATTGCAATTGTGAA |
| | TTGTTGTGGTTGAGGCGTCTGTCCAGAGAAGATGACTTAGAGACCTGTGCTTCTCCTC |
| | CACTTTTAACTGGCCGCTACTTTTGGTCAATTCCTGAAGAAGAGTTTTTGTGTGAGCC |
| | TCCTCTCATTACTCGTCATACACATGAGATGAGAGTCCTGGAGGGACAAAGGGCAACA |
| | CTGAGGTGCAAAGCCAGGGGAGACCCTGAGCCTGCAATTCACTGGATTTCTCCTGAAG |
| | GGAAGCTTATTTCAAATGCAACAAGATCTCTGGTGTATGATAACGGAACACTTGACAT |
| | TCTTATCACAACTGTAAAGGATACAGGTGCTTTTACCTGCATTGCTTCCAATCCTGCT |
| | GGGGAAGCAACACAAATAGTGGATCTTCATATAATTAAGCTCCCTCACTTACTAAATA |
| | GTACAAACCATATCCATGAGCCTGATCCTGGTTCTTCAGATATCTCAACTTCTACCAA |
| | GTCAGGTTCTAATACAAGCAGTAGTAATGGTGATACTAAATTGAGTCAAGATAAAATT |
| | GTGGTGGCAGAAGCTACATCATCAACGGCACTACTTAAATTTAATTTTCGAAGAAATA |
| | TCCCTGGAATACGTATGTTTCAAATCCAGTACAATGGTACTTATGATGACACCCTTGT |
| | TTACAGAATGATACCTCCTACGAGCAAAACTTTTCTGGTCAATAATCTGGCTGCTGGA |
| | ACTATGTATGACTTGTGTGTCTTGGCCATATATGATGATGGCATCACTTCCCTCACTG |
| | CCACAAGAGTCGTGGGTTGCATCCAGTTTACTACGGAACAGGATTATGTGCGTTGCCA |
| | TTTCATGCAGTCCCAGTTTTTGGGAGGCACCCTCGAG |

| | | |
|---|---|---|
| ORF Start: at 1 | | ORF Stop: end of sequence |
| SEQ ID NO: 84 | 515 aa | MW at 57372.8kD |

| | |
|---|---|
| NOV30c, | GSQICPKRCVCQILSPNLATLCAKKGLLFVPPNIDRRTVELRLADNFVTNIKRKDFAN |
| 210086373 | |
| Protein Sequence | MTSLVDLTLSRNTISFITPHAFADLRNLRALHLNSNRLTKITNDMFSGLSNLHHLILN |
| | NNQLTLISSTAFDDVFALEELDLSYNNLETIPWDAVEKMVSLHTLSLDHNMIDNIPKG |
| | TFSHLHKMTRLDVTSNKLQKLPPDPLFQRAQVLATSGIISPSTFALSFGGNPLHCNCE |
| | LLWLRRLSREDDLETCASPPLLTGRYFWSIPEEEFLCEPPLITRHTHEMRVLEGQRAT |
| | LRCKARGDPEPAIHWISPEGKLISNATRSLVYDNGTLDILITTVKDTGAFTCIASNPA |
| | GEATQIVDLHIIKLPHLLNSTNHIHEPDPGSSDISTSTKSGSNTSSSNGDTKLSQDKI |
| | VVAEATSSTALLKFNFRRNIPGIRMFQIQYNGTYDDTLVYRMIPPTSKTFLVNNLAAG |
| | TMYDLCVLAIYDDGITSLTATRVVGCIQFTTEQDYVRCHFMQSQFLGGTLE |

| | |
|---|---|
| SEQ ID NO: 85 | 1545 bp |

| | |
|---|---|
| NOV30d, | GGATCCCAGATCTGTCCAAAGCGTTGTGTCTGTCAGATTTTGTCTCCTAATCTTGCAA |
| 210086403 | |
| DNA Sequence | CCCTTTGTGCCAAGAAAGGGCTTTTATTTGTTCCACCAAACATTGACAGAAGAACTGT |
| | GGAACTGCGGTTGGCAGACAATTTTGTTACAAATATTAAAAGGAAAGATTTTGCCAAT |
| | ATGACCAGCTTGGTGGACCTGACTCTATCCAGGAATACAATAAGTTTTATTACACCTC |
| | ATGCTTTCGCTGACCTACGAAATTTGAGGGCTTTGCATTTGAATAGCAACAGATTGAC |
| | TAAAATTACAAATGATATGTTCAGTGGTCTTTCCAATCTTCATCATTTGATACTGAAC |
| | AACAATCAGCTGACTTTAATTTCCTCTACAGCGTTTGATGATGTCTTCACCCTTGAGG |
| | AGCTGGATCTGTCCTATAATAATCTAGAAACCATTCCTTGGGATGCTGTTGAGAAGAT |
| | GGTTAGCTTGCATACCCTTAGTTTGGATCACAATATGATTGATAACATTCCTAAGGGG |
| | ACCTTCTCCCATTTGCACAAGATGACTCGGTTAGATGTGACATCAAATAAATTGCAGA |
| | AGCTACCACCTGACCCTCTCTTTCAGCGAGCTCAGGTACTAGCAACCTCAGGAATCAT |
| | AAGCCCATCTACTTTTGCATTAAGTTTTGGTGGAAACCCCTTGCATTGCAATTGTGAA |
| | TTGTTGTGGTTGAGGCGTCTGTCCAGAGAAGATGACTTAGAGACCTGTGCTTCTCCTC |
| | CACTTTTAACTGGCCGCTACTTTTGGTCAATTCCTGAAGAAGAGTTTTTGTGTGAGCC |
| | TCCTCTCATTACTCGTCATACACATGAGATGAGAGTCCTGGAGGGACAAAGGGCAACA |
| | CTGAGGTGCAAAGCCAGGGGAGACCCTGAGCCTGCAATTCACTGGATTTCTCCTGAAG |
| | GGAAGCTTATTTCAAATGCAACAAGATCTCTGGTGTATGATAACGGAACACTTGACAT |
| | TCTTATCACAACTGTAAAGGATACAGGTGCTTTTACCTGCATTGCTTCCAATCCTGCT |
| | GGGGAAGCAACACAAATAGTGGATCTTCATATAATTAAGCTCCCTCACTTACTAAATA |
| | GTACAAACCATATCCATGAGCCTGATCCTGGTTCTTCAGATATCTCAACTTCTACCAA |
| | GTCAGGTTCTAATACAAGCAGTAGTAATGGTGATACTAAATTGAGTCAAGATAAAATT |
| | GTGGTGGCAGAAGCTACATCATCAACGGCACTACTTAAATTTAATTTTCAAAGAAATA |
| | TCCCTGGAATACGTATGTTTCAAATCCAGTACAATGGTACTTATGATGACACCCTTGT |
| | TTACAGAATGATACCTCCTACGAGCAAAACTTTTCTGGTCAATAATCTGGCTGCTGGA |
| | ACTATGTATGACTTGTGTGTCTTGGCCATATATGATGATGGCATCACTTCCCTCACTG |
| | CCACAAGAGTCGTGGGTTGCATCCAGTTTACTACGGAACAGGATTATGTGCGTTGCCA |
| | TTTCATGCAGTCCCAGTTTTTGGGAGGCACCCTCGAG |

| | | |
|---|---|---|
| ORF Start: at 1 | | ORF Stop: end of sequence |
| SEQ ID NO: 86 | 515 aa | MW at 57374.8kD |

| | |
|---|---|
| NOV30d, | GSQICPKRCVCQILSPNLATLCAKKGLLFVPPNIDRRTVELRLADNFVTNIKRKDFAN |
| 210086403 | |
| Protein Sequence | MTSLVDLTLSRNTISFITPHAFADLRNLRALHLNSNRLTKITNDMFSGLSNLHHLILN |
| | NNQLTLISSTAFDDVFTLEELDLSYNNLETIPWDAVEKMVSLHTLSLDHNMIDNIPKG |
| | TFSHLHKMTRLDVTSNKLQKLPPDPLFQRAQVLATSGIISPSTFALSFGGNPLHCNCE |
| | LLWLRRLSREDDLETCASPPLLTGRYFWSIPEEEFLCEPPLITRHTHEMRVLEGQRAT |
| | LRCKARGDPEPAIHWISPEGKLISNATRSLVYDNGTLDILITTVKDTGAFTCIASNPA |
| | GEATQIVDLHIIKLPHLLNSTNHIHEPDPGSSDISTSTKSGSNTSSSNGDTKLSQDKI |
| | VVAEATSSTALLKFNFQRNIPGIRMFQIQYNGTYDDTLVYRMIPPTSKTFLVNNLAAG |
| | TMYDLCVLAIYDDGITSLTATRVVGCIQFTTEQDYVRCHFMQSQFLGGTLE |

| | |
|---|---|
| SEQ ID NO: 87 | 1545bp |

| | |
|---|---|
| NOV30e, | GGATCCCAGATCTGTCCAAAGCGTTGTGTCTGTCAGATTTTGTCTCCTAATCTTGCAA |
| 210086422 | |
| DNA Sequence | CCCTTTGTGCCAAGAAAGGGCTTTTATTTGTTCCACCAAACATTGACAGAAGAACTGT |
| | GGAACTGCGGTTGGCAGACAATTTTGTTACAAATATTAAAAGGAAAGATTTTGCCAAT |
| | ATGACCAGCTTGGTGGACCTGACTCTATCCAGGAATACAATAAGTTTTATTACACCTC |
| | ATGCTTTCGCTGACCTACGAAATTTGAGGGCTTTGCATTTGAATAGCAACAGATTGAC |
| | TAAAATTACAAATGATATGTTCAGTGGTCTTTCCAATCTTCATCATTTGATACTGAAC |
| | AACAATCAGCTGACTTTAATTTCCTCTACAGCGTTTGATGATGTCTTCACCCTTGAGG |
| | AGCTGGATCTGTCCTATAATAATCTAGAAACCATTCCTTGGGATGCTGTTGAGAAGAT |
| | GGTTAGCTTGCATACCCTTAGTTTGGATCACAATATGATTGATAACATTCCTAAGGGG |
| | ACCTTCTCCCATTTGCACAAGATGACTCGGTTAGATGTGACATCAAATAAATTGCAGA |
| | AGCTACCACCTGACCCTCTCTTTCAGCGAGCTCAGGTACTAGCAACCTCAGGAATCAT |
| | AAGCCCATCTACTTTTGCATTAAGTTTTGGTGGAAACCCCTTGCATTGCAATTGTGAA |
| | TTGTTGTGGTTGAGGCGTCTGTCCAGAGAAGATGACTTAGAGACCTGTGCTTCTCCTC |
| | CACTTTTAACTGGCCGCTACTTTTGGTCAATTCCTGAAGAAGAGTTTTTGTGTGAGCC |
| | TCCTCTCATTACTCGTCATACACATGAGATGAGAGTCCTGGAGGGACAAAGGGCAACA |
| | CTGAGGTGCAAAGCCAGGGGAGACCCTGAGCCTGCAATTCACTGGATTTCTCCTGAAG |
| | GGAAGCTTATTTCAAATGCAACAAGATCTCTGGTGTATGATAACGGAACACTTGACAT |
| | TCTTATCACAACTGTAAAGGATACAGGTGCTTTTACCTGCATTGCTTCCAATCCTGCT |
| | GGGGAAGCAACACAAATAGTGGATCTTCATATAATTAAGCTCCCTCACTTACTAAATA |
| | GTACAAACCATATCCATGAGCCTGATCCTGGTTCTTCAGATATCTCAACTTCTACCAA |
| | GTCAGGTTCTAATACAAGCAGTAGTAATGGTGATACTAAATTGAGTCAAGATAAAATT |
| | GTGGTGGCAGAAGCTACATCATCAACGGCACTACTTAAATTTAATTTTCAAAGAAATA |
| | TCCCTGGAATACGTATGTTTCAAATCCAGTACAATGGTACTTATGATGACACCCTTGT |
| | TCACAGAATGATACCTCCTACGAGCAAAACTTTTCTGGTCAATAATCTGGCTGCTGGA |
| | ACTATGTATGACTTGTGTGTCTTGGCCATATATGATGATGGCATCACTTCCCTCACTG |
| | CCACAAGAGTCGTGGGTTGCATCCAGTTTACTACGGAACAGGATTATGTGCGTTGCCA |
| | TTTCATGCAGTCCCAGTTTTTGGGAGGCACCCTCGAG |

| | | |
|---|---|---|
| ORF Start: at 1 | | ORF Stop: end of sequence |
| SEQ ID NO: 88 | 515 aa | MW at 57348.7 kD |

| | |
|---|---|
| NOV30e, | GSQICPKRCVCQILSPNLATLCAKKGLLFVPPNIDRRTVELRLADNFVTNIKRKDFAN |
| 210086422 | |
| Protein Sequence | MTSLVDLTLSRNTISFITPHAFADLRNLRALHLNSNRLTKITNDMFSGLSNLHHLILN |
| | NNQLTLISSTAFDDVFTLEELDLSYNNLETIPWDAVEKMVSLHTLSLDHNMIDNIPKG |
| | TFSHLHKMTRLDVTSNKLQKLPPDPLFQRAQVLATSGIISPSTFALSFGGNPLHCNCE |
| | LLWLRRLSREDDLETCASPPLLTGRYFWSIPEEEFLCEPPLITRHTHEMRVLEGQRAT |
| | LRCKARGDPEPAIHWISPEGKLISNATRSLVYDNGTLDILITTVKDTGAFTCIASNPA |
| | GEATQIVDLHIIKLPHLLNSTNHIHEPDPGSSDISTSTKSGSNTSSSNGDTKLSQDKI |
| | VVAEATSSTALLKFNFQRNIPGIRMFQIQYNGTYDDTLVHRMIPPTSKTFLVNNLAAG |
| | TMYDLCVLAIYDDGITSLTATRVVGCIQFTTEQDYVRCHFMQSQFLGGTLE |

Sequence comparison of the above protein sequences yields the following sequence relationships shown in Table 30B.

TABLE 30B
Comparison of NOV30a against NOV30b through NOV30e.

| Protein | NOV30a Residues/ | Identities/Similarities for |
|---|---|---|
| Sequence | Match Residues | the Matched Region |
| NOV30b | 1 . . . 704 | 624/704 (88%) |
| | 1 . . . 704 | 626/704 (88%) |
| NOV30c | 17 . . . 529 | 465/513 (90%) |
| | 2 . . . 514 | 468/513 (90%) |
| NOV30d | 17 . . . 529 | 465/513 (90%) |
| | 2 . . . 514 | 467/513 (90%) |
| NOV30e | 17 . . . 529 | 464/513 (90%) |
| | 2 . . . 514 | 467/513 (90%) |

Further analysis of the NOV30a protein yielded the following properties shown in Table 30C.

TABLE 30C
Protein Sequence Properties NOV30a

| | |
|---|---|
| PSort | 0.6850 probability located in endoplasmic |
| analysis: | reticulum (membrane); |
| | 0.6400 probability located in plasma membrane; |
| | 0.4600 probability located in Golgi body; |
| | 0.1000 probability located in endoplasmic reticulum (lumen) |
| SignalP | Cleavage site between residues 18 and 19 |
| analysis: | |

A search of the NOV30a protein against the Geneseq database, a proprietary database that contains sequences published in patents and patent publication, yielded several homologous proteins shown in Table 30D.

TABLE 30D
Geneseq Results for NOV30a

| | | NOV30a | Identities/ | |
|---|---|---|---|---|
| | | Residues/ | Similarities for | |
| Geneseq | Protein/Organism/Length [Patent | Match | the Matched | Expect |
| Identifier | #, Date] | Residues | Region | Value |
| AAG67505 | Amino acid sequence of a human | 1 . . . 766 | 765/766 (99%) | 0.0 |
| | secreted polypeptide - Homo | 1 . . . 766 | 766/766 (99%) | |
| | sapiens, 766 aa. [WO200166690- | | | |
| | A2, 13-SEP-2001] | | | |
| AAB09968 | Human brain-specific | 1 . . . 756 | 374/780 (47%) | 0.0 |
| | transmembrane glycoprotein - | 1 . . . 770 | 505/780 (63%) | |
| | Homo sapiens, 789 aa. | | | |
| | [WO200031256-A1, 02-JUN-2000] | | | |
| AAM39059 | Human polypeptide SEQ ID NO | 1 . . . 756 | 373/780 (47%) | 0.0 |
| | 2204 - Homo sapiens, 789 aa. | 1 . . . 770 | 504/780 (63%) | |
| | [WO200153312-A1, 26-JUL-2001] | | | |
| AAU28092 | Novel human secretory protein, Seq | 1 . . . 756 | 373/780 (47%) | 0.0 |
| | ID No 261 - Homo sapiens, 789 aa. | 1 . . . 770 | 504/780 (63%) | |
| | [WO200166689-A2, 13-SEP-2001] | | | |
| AAB12448 | Human hh00149 protein SEQ ID | 20 . . . 756 | 368/760 (48%) | 0.0 |
| | NO: 4 - Homo sapiens, 785 aa. | 17 . . . 766 | 497/760 (64%) | |
| | [WO200031255-A1, 02-JUN-2000] | | | |

In a BLAST search of public sequence databases, the NOV30a protein was found to have homology to the proteins shown in the BLASTP data in Table 30E.

TABLE 30E
Public BLASTP Results for NOV30a

| | | NOV30a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q96NI6 | CDNA FLJ30803 FIS, CLONE | 1 . . . 704 | 699/704 (99%) | 0.0 |
| | FEBRA2001245, WEAKLY | 1 . . . 704 | 701/704 (99%) | |
| | SIMILAR TO NAG14 - Homo | | | |
| | sapiens (Human), 719 aa. | | | |
| Q9ULH4 | KIAA1246 PROTEIN - Homo | 1 . . . 756 | 374/780 (47%) | 0.0 |
| | sapiens (Human), 832 aa (fragment). | 44 . . . 813 | 505/780 (63%) | |
| Q9BE71 | HYPOTHETICAL 84.7 KDA | 1 . . . 756 | 374/780 (47%) | 0.0 |
| | PROTEIN - Macaca fascicularis | 1 . . . 770 | 503/780 (63%) | |
| | (Crab eating macaque) | | | |
| | (Cynomolgus monkey), 789 aa. | | | |
| Q9CYK3 | 5730420O05RIK PROTEIN - Mus | 1 . . . 756 | 378/783 (48%) | 0.0 |
| | musculus (Mouse), 788 aa. | 1 . . . 769 | 506/783 (64%) | |
| Q9P244 | KIAA1484 PROTEIN - Homo | 58 . . . 701 | 332/655 (50%) | e−177 |
| | sapiens (Human), 700 aa (fragment). | 1 . . . 631 | 441/655 (66%) | |

PFam analysis predicts that the NOV30a protein contains the domains shown in the Table 30F.

TABLE 30F
Domain Analysis of NOV30a

| | NOV30a | Identities/Similarities | Expect |
|---|---|---|---|
| Pfam Domain | Match Region | for the Matched Region | Value |
| LRR | 76 . . . 99 | 9/25 (36%) | 0.49 |
| | | 18/25 (72%) | |
| LRR | 100 . . . 123 | 8/25 (32%) | 0.0031 |
| | | 21/25 (84%) | |
| LRR | 148 . . . 171 | 11/25 (44%) | 0.0021 |
| | | 20/25 (80%) | |
| LRR | 172 . . . 195 | 7/25 (28%) | 0.0035 |
| | | 19/25 (76%) | |
| LRRCT | 240 . . . 285 | 21/54 (39%) | 0.00023 |
| | | 37/54 (69%) | |
| ig | 301 . . . 359 | 15/62 (24%) | 1.4e−08 |
| | | 41/62 (66%) | |
| fn3 | 416 . . . 496 | 16/86 (19%) | 0.066 |
| | | 57/86 (66%) | |

### Example 31

The NOV31 clone was analyzed, and the nucleotide and encoded polypeptide sequences are shown in Table 31A.

TABLE 31A
NOV31 Sequence Analysis

| | |
|---|---|
| SEQ ID NO: 89 | 1220 bp |

| | |
|---|---|
| NOV31a, | TCATGGCGCTGGCGGGCTTGGCCATGGGCTGCATCGACACCGTGGCCAGCATGCAGCT |
| CG108184-01 | |
| DNA Sequence | GGTAAGGATGTACCAGAAGGACTCGGCCGTCTTCCTCCAGGTGCTCCATTTCTTCGTG |
| | GGCTTTGGTGCTCTGCTGAGCCCCCTTATTGCTGACCCTTTCCTGTCTGAGGCCAACT |
| | GCTTGCCTGCCAATAGCACGGCCAACACCACCTCCCGAGGCCACCTGTTCCATGTCTC |
| | CAGGGTGCTGGGCCAGCACCACGTAGATGCCAAGCCTTGGTCCAACCAGACGTTCCCA |
| | GGGCTGACTCCAAAGGACGGGGCAGGGACCCGAGTGTCCTATGCCTTCTGGATCATGG |
| | CCCTCATCAATCTTCCAGTGCCCATGGCTGTGCTGATGCTGCTGTCCAAGGAGCGGCT |
| | GCTGACCTGCTGTCCCCAGAGGAGGCCCCTGCTTCTGTCTGCTGATGAGCTTGCCTTG |
| | GAGACACAGCCTCCTGAGAASGAAGATGCCTCCTCACTGCCCCCAAAGTTTCAGTCAC |
| | ACCTAGGTCATGAGGACCTGTTCAGCTGCTGCCAAAGGAAGAACCTCAGAGGAGCCCC |
| | TTATTCCTTCTTTGCCATCCACATCACGGGCGCCCTGGTACTGTTCATGACGGATGGG |
| | TTGACGGGTGCCTATTCCGCCTTCGTGTACAGCTATGCTGTGGAGAAGCCCCTGTCTG |
| | TGGGACACAAGGTGGCTGGCTACCTCCCCAGCCTCTTCTGGGGCTTCATCACACTGGG |
| | CCGGCTCCTCTCCATTCCCATATCCTCAAGAATGAAGCCGGCCACCATCGTTTTCATC |
| | AACGTGGTAGGCGTGGTGGTGACGTTCCTGGTGCTGCTTATTTTCTCCTACAACGTCG |
| | TGTTCCTGTTCGTGGGGACGGCAAGCCTGGGCCTGTTTCTCAGCAGCACCTTCCCCAG |
| | CATGCTGGCCTACACGGAGGACTCGCTGCAGTACAAAGGCTGTGCAACCACAGTGCTG |
| | GTGACAGGGGCAGGAGTTGGCGAGATGGTGCTGCAGATGCTGGTTGGTTCGATATTCC |
| | AGGCTCAGGGCAGCTATAGTTTCCTGGTCTGTGGCGTGATCTTTGCTTGTCTGGCTTT |
| | TACCTTCTATATCTTGCTCCTGTTTTTCCACAGGATGCACCCTGGACTCCCATCAGTT |
| | CCTACCCAAGACAGATCAATTGGAATGGAAAACTCTGAGTGCTACCAGAGGTAAAACT |
| | GG |

| | |
|---|---|
| ORF Start: ATG at 3 | ORF Stop: TAA at 1212 |

| | | |
|---|---|---|
| SEQ ID NO: 90 | 403 aa | MW at 44063.2kD |

| | |
|---|---|
| NOV31a, | MALAGLAMGCIDTVANMQLVRMYQKDSAVFLQVLHFFVGFGALLSPLIADPFLSEANC |
| CG108184-01 | |
| Protein Sequence | LPANSTANTTSRGHLFHVSRVLGQHHVDAKPWSNQTFPGLTPKDGAGTRVSYAFWIMA |
| | LINLPVPMAVLMLLSKERLLTCCPQRRPLLLSADELALETQPPEKEDASSLPPKFQSH |
| | LGHEDLFSCCQRKNLRGAPYSFFAIHITGALVLFMTDGLTGAYSAFVYSYAVEKPLSV |
| | GHKVAGYLPSLFWGFITLGRLLSIPISSRMKPATMVFINVVGVVVTFLVLLIFSYNVV |
| | FLFVGTASLGLFLSSTFPSMLAYTEDSLQYKGCATTVLVTGAGVGEMVLQMLVGSIFQ |
| | AQGSYSFLVCGVIFGCLAFTFYILLLFFHRMHPGLPSVPTQDRSIGMENSECYQR |

| | |
|---|---|
| SEQ ID NO: 91 | 1217 bp |

| | |
|---|---|
| NOV31b, | ATGGCGCTGGCGGGCTTGGCCATGGGCTGCATCGACACCGTGGCCAACATGCAGCAGG |
| CG108184-02 | |
| DNA SEQUENCE | TAAGGATGTACCAGAAGGACTCGGCCGTCTTCCTCCAGGTGCTCCATTTCTTCGTGGG |
| | CTTTGGTGCTCTGCTGAGCCCCCTTATTGCTGACCCTTTCCTGTCTGAGGCCAACTGC |
| | TTGCCTGCCAATAGCACGGCCAACACCACCTCCCGAGGCCACCTGTTCCATGTCTCCA |
| | GGGTGCTGGGCCAGCACCACGTAGATGCCAAGCCTTGGTCCAACCAGACGTTCCCAGG |
| | GCTGACTCCAAAGGACGGGGCAGGGACCCGAGTGTCCTATGCCTTCTGGATCATGGCC |
| | CTCATCAATCTTCCAGTGCCCATGGCTGTGCTGATGCTGCTGTCCAAGGAGCGGCTGC |
| | TGACCTGCTGTCCCCAGAGGAGGCCCCTGCTTCTGTCTGCTGATGAGCTTGCCTTGGA |
| | GACACAGCCTCCTGAGAAGGAAGATGCCTCCTCACTGCCCCCAAAGTTTCAGTCACAC |
| | CTAGGGCATGAGGACCTGTTCAGCTGCTGCCAAAGGAAGAACCTCAGAGGAGCCCCTT |
| | ATTCCTTCTTTGCCATCCACATCACGGCCGCCCTGGTCCTGTTCATGACGGATGGGTT |
| | GACGGGTGCCTATTCCGCCTTCGTGTACAGCTATGCTGTGGAGAAGCCCCTGTCTGTG |
| | GGACACAAGGTGGCTGGCTACCTCCCCAGCCTCTTCTGGGGCTTCATCACACTGGGCC |
| | GGCTCCTCTCCATTCCCATATCCTCAAGAATGAAGCCGGCCACCATGGTTTTCATCAA |
| | CGTGGTTGGCGTGGTGGTGACGTTCCTGGTGCTGCTTATTTTCTCCTACAACGTCGTC |
| | TTCCTGTTCGTGGGGACGGCAAGCCTGGGCCTGTTTCTCAGCAGCACCTTCCCCAGCA |
| | TGCTGGCCTACACGGAGGACTCGCTGCAGTACAAAGGCTGTGCAACCACAGTGCTGGT |
| | GACAGGGGCAGGAGTTGGCGAGATGGTGCTGCAGATGCTGGTTGGTTCGATATTCCAG |
| | GCTCAGGGCAGCTATAGTTTCCTGGTCTGTGGCGTGATCTTTGGTTGTCTGGCTTTTA |
| | CCTTCTATATCTTGCTCCTGTTTTTCCACAGGATGCACCCTGGACTCCCATCAGTTCC |
| | TACCCAAGACAGATCAATTGGAATGGAAAACTCTGAGTGCTACCAGAGGTAAAACTG |

| | |
|---|---|
| ORF Start: ATG at 1 | ORF Stop: TAA at 1210 |

| | | |
|---|---|---|
| SEQ ID NO: 92 | 403 aa | MW at 44092.2kD |

| | |
|---|---|
| NOV31b, | MALAGLAMGCIDTVANMQQVRMYQKDSAVFLQVLHFFVGFGALLSPLIADPFLSEANC |
| CG108184-02 | |
| Protein Sequence | LPANSTANTTSRGHLFHVSRVLGQHHVDAKPWSNQTFPGLTPKDGAGTRVSYAFWIMA |
| | LINLPVPMAVLMLLSKERLLTCCPQRRPLLLSADELALETQPPEKEDASSLPPKFQSH |
| | LGHEDLFSCCQRKNLRGAPYSFFAIHITAALVLFMTDGLTGAYSAFVYSYAVEKPLSV |
| | GHKVAGYLPSLFWGFITLGRLLSIFISSRMKPATMVFINVVGVVVTFLVLLIFSYNVV |
| | FLFVGTASLGLFLSSTFPSMLAYTEDSLQYKGCATIWLVTGAGVGEMVLQMLVGSIFQ |
| | AQGSYSFLVCGVIFGCLAFTFYILLLFFHRMHPGLPSVPTQDRSIGMENSECYQR |

| | |
|---|---|
| SEQ ID NO: 93 | 1190 bp |

| | |
|---|---|
| NOV31b, | ATGGCGCTGGCGGGCTTGGCCATGGGCTGCATCGACACCGTGGCCAACATGCAGCTGG |
| CG108184-03 | |
| DNA Sequence | TAAGGATGTACCAGAAGGACTCGGCCGTCTTCCTCCAGGTGCTCCATTTCTTCGTGGG |
| | CTTTGGTGCTCTGCTGAGCCCCCTTATTGCTGACCCTTTCCTGTCTGAGGCCAACTGC |
| | TTGCCTGCCAATAGCACGGCCAACACCACCTCCCGAGGCCACCTGTTCCATGTCTCCA |
| | GGGTGCTGGGCCAGCACCACGTAGATGCCAAGCCTTGGTCCAACCAGACGTTCCCAGG |
| | GCTGACTCCAAAGGACGGGGCAGGGACCCGAGTGTCCTATGCCTTCTGGATCATGGCC |
| | CTCATCAATCTTCCAGTGCCCATGGCTGTGCTGATGCTGCTGTCCAAGGAGCGGCTGC |
| | TGACCTGCTGTCCCCAGAGGAGGCCCCTGCTTCTGTCTGCTGATGAGCTTGCCTTGGA |
| | GACACAGCCTCCTGAGAAGGAAGATGCCTCCTCACTGCCCCCAAAGTTTCAGTCACAC |
| | CTAGGGCATGAGGACCTGTTCAGCTGCTGCCAAAGGAAGAACCTCAGAGGAGCCCCTT |
| | ATTCCTTCTTTGCCATCCACATCACGGGCGCCCTGGGTGCCTATTCCGCCTTCGTGTA |
| | CAGCTATGCTGTGGAGAAGCCCCTGTCTGTGGGACACAAGGTGGCTGGCTACCTCCCC |
| | AGCCTCTTCTGGGGCTTCATCACACTGGGCCGGCTCCTCTCCATTCCCATATCCTCAA |
| | GAATGAAGCCGGCCACCATGGTTTTCATCAACGTGGTTGGCGTGGTGGTGACGTTCCT |
| | GGTGCTGCTTATTTTCTCCTACAACGTCGTCTTCCTGTTCGTGGGGACGGCAAGCCTG |
| | GGCCTGTTTCTCAGCAGCACCTTCCCCAGCATGCTGGCCTACACGGAGGACTCGCTGC |
| | AGTACAAAGGCTGTGCAACCACAGTGCTGGTGACAGGGGCAGGAGTTGGCGAGATGGT |
| | GCTGCAGATGCTGGTTGGTTCGATATTCCAGGCTCAGGGCAGCTATAGTTTCCTGGTC |
| | TGTGGCGTGATCTTTGGTTGTCTGGCTTTTACCTTCTATACCTTGCTCCTGTTTTTCC |
| | ACAGGATGCACCCTGGACTCCCATCAGTTCCTACCCAAGACAGATCAATTGGAATGGA |
| | AAACTCTGAGTGCTACCAGAGGTAAAACTG |

| | |
|---|---|
| ORF Start: ATG at 1 | ORF Stop: TAA at 1183 |

| | | |
|---|---|---|
| SEQ ID NO: 94 | 399 aa | MW at 43072.9kD |

| | |
|---|---|
| NOV31c, | MALAGLAMGCIDTVANMQLVRMYQKDSAVFLQVLHFFVGFGALLSPLIADPFLSEANC |
| CG108184-03 | |
| Protein Sequence | LPANSTANTTSRGHLFHVSRVLGQHHVDAKPWSNQTFPGLTPKDGAGTRVSYAFWIMA |
| | LINLPVPMAVLMLLSKERLLTCCPQRRPLLLSADELALETQPPEKEDASSLPPKFQSH |
| | LGHEDLFSCCQRKNLRGAPYSFFAIHITGALGAYSAFVYSYAVEKPLSVGHKVAGYLP |
| | SLFWGFITLGRLLSIPISSRMKPATMVFINVVGVVVTFLVLLIFSYNVVFLFVGTASL |
| | GLFLSSTFPSMLAYTEDSLQYKGCATTVLVTGAGVGEMVLQMLVGSIFQAQGSYSFLV |
| | CGVIFGCLAFTFYTLLLFFHRMHPGLPSVPTQDRSIGMENSECYQR |

Sequence comparison of the above protein sequences yields the following sequence relationships shown in Table 31B.

TABLE 31B
Comparison of NOV31a against NOV31b and NOV31c.

| | NOV31a Residues/ | Identities/Similarities |
|---|---|---|
| Protein Sequence | Match Residues | for the Matched Region |
| NOV31b | 1 . . . 403 | 383/403 (95%) |
| | 1 . . . 403 | 383/403 (95%) |
| NOV31c | 1 . . . 403 | 364/403 (90%) |
| | 1 . . . 394 | 364/403 (90%) |

Further analysis of the NOV31a protein yielded the following properties shown in Table 31C.

TABLE 31C
Protein Sequence Properties NOV31a

| | |
|---|---|
| PSort | 0.6000 probability located in plasma membrane; 0.4445 |
| analysis: | probability located in mitochondrial inner membrane; 0.4000 |
| | probability located in Golgi body; 0.3000 probability |
| | located in endoplasmic reticulum (membrane) |
| SignalP | Cleavage site between residues 50 and 51 |
| analysis: | |

A search of the NOV31a protein against the Geneseq database, a proprietary database that contains sequences published in patents and patent publication, yielded several homologous proteins shown in Table 31D.

TABLE 31D
Geneseq Results for NOV31a

| | | | Identities/ | |
|---|---|---|---|---|
| | | NOV31a | Similarities | |
| | | Residues/ | for the | |
| Geneseq | Protein/Organism/Length [Patent | Match | Matched | Expect |
| Identifier | #, Date] | Residues | Region | Value |
| AAM24206 | Human EST encoded protein SEQ ID | 1 . . . 101 | 76/109 (69%) | 9e−32 |
| | NO: 1731 - Homo sapiens, 226 aa. | 112 . . . 220 | 81/109 (73%) | |
| | [WO200154477-A2, 02-AUG-2001] | | | |
| AAY11034 | H. pylori ORF | 201 . . . 346 | 32/146 (21%) | 0.35 |
| | 04ep41903_19689182_c1_43 inner | 226 . . . 368 | 68/146 (45%) | |
| | membrane protein - Helicobacter | | | |
| | pylori, 407 aa. [WO9824475-A1, 11-JUN-1998] | | | |
| AAY11033 | H. pylori ORF | 201 . . . 346 | 32/146 (21%) | 0.35 |
| | 04ep41903_16667055_c1_37 inner | 131 . . . 273 | 68/146 (45%) | |
| | membrane protein - Helicobacter | | | |
| | pylori, 312 aa. [WO9824475-A1, 11-JUN-1998] | | | |
| ABB68766 | Drosophila melanogaster polypeptide | 212 . . . 371 | 39/168 (23%) | 0.46 |
| | SEQ ID NO: 33090 - Drosophila | 582 . . . 746 | 71/168 (42%) | |
| | melanogaster, 816 aa. | | | |
| | [WO200171042-A2, 27-SEP-2001] | | | |
| ABB48281 | Listeria monocytogenes protein #985 - | 250 . . . 356 | 32/117 (27%) | 1.0 |
| | Listeria monocytogenes, 402 aa. | 62 . . . 172 | 52/117 (44%) | |
| | [WO200177335-A2, 18-OCT-2001] | | | |

In a BLAST search of public sequence databases, the NOV31a protein was found to have homology to the proteins shown in the BLASTP data in Table 31E.

TABLE 31E
Public BLASTP Results for NOV31a

| | | NOV31a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9GM43 | HYPOTHETICAL 44.1 KDA | 1 . . . 403 | 397/403 (98%) | 0.0 |
| | PROTEIN - Macaca fascicularis | 1 . . . 403 | 399/403 (98%) | |
| | (Crab eating macaque) | | | |
| | (Cynomolgus monkey), 403 aa. | | | |
| CAD28481 | HYPOTHETICAL 34.1 KDA | 93 . . . 403 | 311/311 (100%) | e−180 |
| | PROTEIN - Homo sapiens | 1 . . . 311 | 311/311 (100%) | |
| | (Human), 311 aa (fragment). | | | |
| Q9D8I5 | 2010001E11RIK PROTEIN - Mus | 205 . . . 375 | 49/171 (28%) | 1e−13 |
| | musculus (Mouse), 366 aa. | 188 . . . 353 | 89/171 (51%) | |
| Q8VCV9 | SIMILAR TO RIKEN CDNA | 205 . . . 375 | 46/171 (26%) | 1e−12 |
| | 2010001E11 GENE - Mus | 187 . . . 352 | 89/171 (51%) | |
| | musculus (Mouse), 377 aa. | | | |
| Q96PW9 | KIAA1919 PROTEIN - Homo | 205 . . . 353 | 44/149 (29%) | 1e−12 |
| | sapiens (Human), 403 aa | 121 . . . 265 | 79/149 (52%) | |
| | (fragment). | | | |

### Example 32

The NOV32 clone was analyzed, and the nucleotide and encoded polypeptide sequences are shown in Table 32A.

TABLE 32A
NOV32 Sequence Analysis

| | |
|---|---|
| SEQ ID NO: 95 | 1557 bp |

| | |
|---|---|
| NOV32a, | CCCTGAGGAACAGACGTTCCCTGGCAGCCCTGGCACCTACAACCCCAGACATGCTGCT |
| CG10823 8-01 | |
| DNA Sequence | GCTGCTGCCCCTGCTCTGGGGGAGGGAGGGGGTGGAGGGACAGGGACAGCAAGAGAAT |
| | GGTTACACACTGCAAGTGCAGAGGGAGGTGAGGGTGCAGGAGGGCCTGTGTGTCCACG |
| | TGCCCTCCTCCTTCTCCCACCCCCAGGTTGCCTTGACTAACTCTCCCCAAGTTCATGG |
| | CTACTGGTTCCAGGAAGGGGCTGACACAGCCCAGGATGCTCCAATGGCTACGAACAAC |
| | CCAAAACGAAAAGTGAAGAAGGAGACCCAGGGCCGATTCCGTCTCTCTGGAAACCTGC |
| | AGATGAACGACTGCTCCCTGAGCATCGGAGACGCCAGGAGGAAGGACCAGGGGTCATT |
| | TTCTTTCTTTCGCATGGAGAGAGGAAGCATGAGATGGAATTACGCGTCTAACCAGCTC |
| | CACGTGTTGGTGACGGCCCTGACCCACAGGCCCAATATCTCCTCCCTGGGGACCATGG |
| | AGTCCGGCCGCCCGGGAAACCTGACCTGCTCTGTGTCCTGGGCCTGTGAGCAGGGGAT |
| | ACCCCTACCATCTATCTCCTGGATGGGGACCTCCGTGTCCTTCCCGGGCCGCACCACA |
| | GCCCGCTCCTCAGTGCTCACCCTCATCCCAAAGCCCCAGGACCATGGCACCAACCTCA |
| | CCTGTCAGGTGACCCTGCCTGAGGCTGGTGTGACCTTGACCAGGACTGTCCAATTCAA |
| | TGCGTCCGACCCTCCTCAGAACTTGACTGTGGCTATCTTCCAAGCAGACGGCACAGCA |
| | TCCACAGCCTTGGGGAACAGCTCATCCCTCTCAGTCCTGGAGGGCCAGTCTCTGCGCC |
| | TGGTCTGTGCTGTCGACAGCAATCCCCCTGCCAGGCTGAGCTGGACCCAAGGGAGCCT |
| | GACCCTGAGCCCCTCACAGTCCTCGAACCATGGGCTGCTGAAGCTGCCTCGAGTGCAC |
| | GCGAGGGATGAAGGGGAATTCACCTGCCGAGCTCAGAACCCTCGGGGCTCCCAGCACA |
| | TTTCCCTGAGCCTCTCCCTGCAGAATGAGGGCACAGGTACCACATGGCCTGTATCAGG |
| | AGTGATGCTGGGGGTGGTCGGGGGAGCTGGAGCCACAGCCCTGGTCTTCCTGTCCTTC |
| | TGCGTCATCTTCATCGTGGTGAGGTCCTGCAGGAAGAAATCAGCAAGGCCAGCAGCGG |
| | GCATCAGGGATATGGGCATGGAGGATGCAAACGCTGTCAGGGGCTCAGCCTATCAGCA |
| | GGGACCCCTGACTGAATCCTGGACAGACGGCAGCCCCCCGAAGCATCCTCCCATGGCT |
| | GCCTCCTCCTTAGGAGAAGGAGAGCTCCAGCATGCAACCCTCAGCTTCCATAAGGTCA |
| | GGCCTCAGAACGCGCAGGGACAGGAGGCCATGGACAGTGAATACTTGGAGATCAAGAT |
| | CCACAAGCGAGAAACTGCAGAGACTCGGGCCTGATTGGGGGATCACGGTCCCTCCAGG |
| | CAAAGGAGAAGTCAGAAGCTGATTCTTCTAAAATTAACAGCCCTCTAGA |

| | |
|---|---|
| ORE Start: ATG at 51 | ORF Stop: TGA at 1482 |

| | | |
|---|---|---|
| SEQ ID NO: 96 | 477 aa | MW at 51555.7kD |

| | |
|---|---|
| NOV32a, | MLLLLPLLWGREGVEGQGQQENGYTLQVQREVRVQEGLCVHVPSSFSHPQVALTNSPQ |
| CG108238-01 | |
| Protein Sequence | VHGYWFQEGADTAQDAPMATNNPKRKVKKETQGRFRLSGNLQMNDCSLSIGDARRKDQ |
| | GSFSFFRMERGSMRWNYASNQLHVLVTALTHRPNISSLGTMESGRPGNLTCSVSWACE |
| | QGIPLPSISWMGTSVSFPGRTTARSSVLTLIPKPQDHGTNLTCQVTLPEAGVTLTRTV |
| | QFNASDPPQNLTVAIFQADGTASTALGNSSSLSVLEGQSLRLVCAVDSNPPARLSWTQ |
| | GSLTLSPSQSSNHGLLKLPRVHARDEGEFTCRAQNPRGSQHISLSLSLQNEGTGTTWP |
| | VSGVMLGVVGGAGATALVFLSFCVIFIVVRSCRKKSARPAAGIRDMGMEDANAVRGSA |
| | YQQGPLTESWTDGSPPKHPPMAASSLGEGELQHATLSFHKVRPQNAQGQEAMDSEYLE |
| | IKIHKRETAETRA |

Further analysis of the NOV32a protein yielded the following properties shown in Table 32B.

TABLE 32B
Protein Sequence Properties NOV32a

| | |
|---|---|
| PSort | 0.7000 probability located in plasma membrane; 0.3000 |
| analysis: | probability located in microbody (peroxisome); 0.2000 |
| | probability located in endoplasmic reticulum |
| | (membrane); 0.1000 probability located in |
| | mitochondrial inner membrane |
| SignalP | Cleavage site between residues 17 and 18 |
| analysis: | |

A search of the NOV32a protein against the Geneseq database, a proprietary database that contains sequences published in patents and patent publication, yielded several homologous proteins shown in Table 32C.

TABLE 32C
Geneseq Results for NOV32a

| | | NOV32a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABB53288 | Human polypeptide #28 - Homo | 1 . . . 477 | 326/479 (68%) | e−180 |
| | sapiens, 490 aa. [WO200181363- | 5 . . . 476 | 374/479 (78%) | |
| | A1, 01-NOV-2001] | | | |
| AAE15809 | Human sialoadhesin factor-3 | 1 . . . 469 | 317/469 (67%) | e−172 |
| | (SAF-3) - Homo sapiens, 467 aa. | 3 . . . 467 | 362/469 (76%) | |
| | [WO200190193-A1, 29-NOV-2001] | | | |
| AAW59992 | Sialoadhesin family member-3 | 1 . . . 469 | 317/469 (67%) | e−172 |
| | (SAF-3) - Homo sapiens, 467 aa. | 3 . . . 467 | 362/469 (76%) | |
| | [EP869178-A1, 07-OCT-1998] | | | |
| AAB29188 | Siglec 5 protein - Homo sapiens, | 1 . . . 469 | 316/469 (67%) | e−171 |
| | 467 aa. [WO200053747-A1, 14-SEP-2000] | 3 . . . 467 | 361/469 (76%) | |
| AAU14582 | Human novel protein #453 - Homo | 1 . . . 469 | 316/469 (67%) | e−171 |
| | sapiens, 467 aa. [WO200155437- | 3 . . . 467 | 361/469 (76%) | |
| | A2, 02-AUG-2001] | | | |

In a BLAST search of public sequence databases, the NOV32a protein was found to have homology to the proteins shown in the BLASTP data in Table 32D.

TABLE 32D
Public BLASTP Results for NOV32a

| | | NOV32a | Identities/ | |
|---|---|---|---|---|
| Protein | | Residues/ | Similarities for | |
| Accession | | Match | the Matched | Expect |
| Number | Protein/Organism/Length | Residues | Portion | Value |
| AAG00573 | SIALIC ACID BINDING | 1 . . . 477 | 351/485 (72%) | 0.0 |
| | IMMUNOGLOBULIN-LIKE | 4 . . . 485 | 396/485 (81%) | |
| | LECTIN 8 LONG SPLICE | | | |
| | VARIANT - Homo sapiens | | | |
| | (Human), 499 aa. | | | |
| Q9Y286 | QA79 MEMBRANE PROTEIN, | 1 . . . 469 | 316/469 (67%) | e−170 |
| | ALLELIC VARIANT AIRM-1B | 3 . . . 467 | 361/469 (76%) | |
| | PRECURSOR - Homo sapiens | | | |
| | (Human), 467 aa. | | | |
| Q9NYZ4 | SIGLEC SAF2 - Homo sapiens | 1 . . . 408 | 299/414 (72%) | e−168 |
| | (Human), 431 aa. | 4 . . . 415 | 338/414 (81%) | |
| AAK51233 | SIALIC ACID-BINDING | 3 . . . 469 | 309/480 (64%) | e−167 |
| | IMMUNOGLOBULIN-LIKE | 1 . . . 477 | 355/480 (73%) | |
| | LECTIN-LIKE SHORT SPLICE | | | |
| | VARIANT - Homo sapiens | | | |
| | (Human), 477 aa. | | | |
| Q9BYI9 | FOAP-9 - Homo sapiens (Human), | 1 . . . 469 | 312/475 (65%) | e−167 |
| | 463 aa. | 2 . . . 463 | 362/475 (75%) | |

PFam analysis predicts that the NOV32a protein contains the domains shown in the Table 32E.

TABLE 32E
Domain Analysis of NOV32a

| | NOV32a | Identities/Similarities | Expect |
|---|---|---|---|
| Pfam Domain | Match Region | for the Matched Region | Value |
| ig | 160 . . . 219 | 18/60 (30%) | 0.023 |
| | | 40/60 (67%) | |
| ig | 269 . . . 323 | 13/58 (22%) | 1.7e−08 |
| | | 45/58 (78%) | |

### Example 33

The NOV33 clone was analyzed, and the nucleotide and encoded polypeptide sequences are shown in Table 33A.

TABLE 33A
NOV33 Sequence Analysis

| | |
|---|---|
| SEQ ID NO: 97 | 1494 bp |

| | |
|---|---|
| NOV33a, | TTTAATGCACAGATATAATACAGGAGTATGCTAGGTGGGTCTGTTTTTCAACTTGGAT |
| CG108695-01 | |
| DNA Sequence | CTCTCCTTACAGGGCTACTTTCTCATGTTTTGATGGAAGTCAAGAAGATACCAGTGGA |
| | AGAGGGGCTGTGCACTACAATCCCTTGTGCATTTGAATTTCCCAGAGAACCCCCAAGC |
| | AATTCCATGATCATGCACTACTGGCTCAACAAAAACATCAGCTCCCTGGTAGCTACAA |
| | ATAAACCCAATGCTACCATTGGTGATAACACCAAGGACAAATTTTACATGACTGGGAA |
| | TCTTGATGAAGAAGACTGTACCCTACTCATCCACGATATACTCAAAGGGAACAGCATA |
| | ACATATTTATTCTACGCAGATCTAGGAGAACAAAAAAGTGCTTTCCTGGGGGAGAATA |
| | TCAAACTTACCCAAAAGCCAGAGCTCCACATGCCAGAGATTCTTTTGCCTGAGAAGAC |
| | TGTGACCTTGAACTGTACCCTCAAAGGCACCTGCAAAGAAACCAAAGCCCTCTTCCAC |
| | TCCCGGAAGAACCCAGCCATCTCCAGCAGCTCCTCCTCGGTGCTGCACTTCACCCTGA |
| | GGCCTGAGGACCATGGCAACACCCTTGGATGTCACTTGAACTTATCCCTAGCCAACGT |
| | TTGTTCAATTCCTCTTGTTCCTTGGAGAAGACAGTTCTGTGCAGCTGTTCCTTCCACG |
| | GGATCCCCACGCCCTCCGTGCAGTGGTGGATGGGAGGTGTCCCTGTGGCTGTGAACAG |
| | CATGGATAACATCCTCCGGGTGACTTCTTCCACATGTGCCCCCTGGGCCAACAGCACC |
| | ATCAGCCTCATTGGGGAGCCAGAAAGAGTCATGAGACTTCACTGTGAGGGGAAGAACC |
| | AATATGGAATTCACACTTCCAGCATCTTCCTGCTTAGAATGAAGATGCTTACCTGGTG |
| | GGAGGAGCATCAGAGTCCCAAGGCCAAAGAAGGCTTGCCCCTTAAGAAACCAGAGCTG |
| | CTGGAGGAAACAGAAGTACCCAAAATGCCTGAAGCTGACACCCCACCAGACCGGGCTG |
| | GAGGTAAGTCCCTGGGACAGTCACAGGCAGACTGTCAAGCAACAGGGCCGCAGGCCAG |
| | GGCTCCCACCCCATCACCTGGCAGAACCCCGGGGAGGCACTTGTTCAGGACGGAGAGA |
| | CCCAGGCCCCCTCGCCCCACACAGCAGATGCTACATTTGCCAAAACAAAGCCCCACAG |
| | ACTGGGCGGCTTGAACGGCAGATACTGATTTTCTCCCCCTCTGGAGGCTGGAAATCCC |
| | AGGTCAAGGTGCCAGCAGCAGAGCTTCCTTCTGAGGCCTCTCACCTTGGCCGGTAGAT |
| | GCTGTCTTCTCCCTGTGTCCTCACAGGGTCATCCCTCTGCGTGTGCCTGGGTCCTAAT |
| | TTCCTTCCCTTGTAAGGACACCAGTCATTGAATTCAGGCCCATC |

| | |
|---|---|
| ORF Start: ATG at 28 | ORF Stop: TGA at 1288 |

| | | |
|---|---|---|
| SEQ ID NO:98 | 420 aa | MW at 46435.0kd |

| | |
|---|---|
| NOV33a, | MLGGSVFQLGSLLTGLLSHVLMEVKKIPVEEGLCTTIPCAFEFPREPPSNSMIMHYWL |
| CG108695-01 | |
| Protein Sequence | NKNISSLVATNKPNATIGDNTKDKFYMTGNLDEEDCTLLIHDILKGNSITYLFYADLG |
| | EQKSAFLGENIKLTQKPELHMPEILLAEKTVTLNCTLKGTCKETKALFHSRKNPAISS |
| | SSSSVLHFTLRPEDHGNTLGCHLNLSLANVTRSSLVKLQVVCECWAPARLFNSSCSLE |
| | KTVLCSCSFHGIPTPSVQWWMGGVPVAVNSMDNILRVTSSTCAPWANSTISLIGEPER |
| | VMRLHCEGKNQYGIHTSSIFLLRMKMLTWWEEHQSPKAKEGLPLKKPELLEETEVPKM |
| | PEADTPPDRAGGKSLGQSQADCQATGPQARAPTPSPGRTPGRHLFRTERPRPPRPTQQ |
| | MLHLPKQSPTDWAA |

Further analysis of the NOV33a protein yielded the following properties shown in Table 33B.

TABLE 33B
Protein Sequence Properties NOV33a

| | |
|---|---|
| PSort | 0.4500 probability located in cytoplasm; 0.3000 probability |
| analysis: | located in microbody (peroxisome); 0.1000 probability located |
| | in mitochondrial matrix space; |
| | 0.1000 probability located in lysosome (lumen) |
| SignalP | Cleavage site between residues 23 and 24 |
| analysis: | |

A search of the NOV33a protein against the Geneseq database, a proprietary database that contains sequences published in patents and patent publication, yielded several homologous proteins shown in Table 33C.

TABLE 33C
Geneseq Results for NOV33a

| | | NOV33a | Identities/ | |
|---|---|---|---|---|
| | | Residues/ | Similarities for | |
| Geneseq | Protein/Organism/Length [Patent | Match | the Matched | Expect |
| Identifier | #, Date] | Residues | Region | Value |
| AAM00948 | Human bone marrow protein, SEQ | 25 . . . 232 | 69/226 (30%) | 6e−21 |
| | ID NO: 424 - Homo sapiens, 556 | 32 . . . 254 | 113/226 (49%) | |
| | aa. [WO200153453-A2, 26-JUL-2001] | | | |
| AAU14239 | Human novel protein #110 - Homo | 25 . . . 232 | 69/226 (30%) | 6e−21 |
| | sapiens, 551 aa. [WO200155437- | 27 . . . 249 | 113/226 (49%) | |
| | A2, 02-AUG-2001] | | | |
| AAW55884 | Human CD33-like protein - Homo | 25 . . . 232 | 69/226 (30%) | 6e−21 |
| | sapiens, 551 aa. [WO9806733-A1, | 27 . . . 249 | 113/226 (49%) | |
| | 19-FEB-1998] | | | |
| AAB50907 | Human PRO333 protein - Homo | 25 . . . 224 | 67/216 (31%) | 7e−21 |
| | sapiens, 394 aa. [WO200073452- | 25 . . . 237 | 108/216 (49%) | |
| | A2, 07-DEC-2000] | | | |
| AAB33462 | Human PRO333 protein UNQ294 | 25 . . . 224 | 67/216 (31%) | 7e−21 |
| | SEQ ID NO: 249 −Homo sapiens, | 25 . . . 237 | 108/216 (49%) | |
| | 394 aa. [WO200053758-A2, 14-SEP-2000] | | | |

In a BLAST search of public sequence databases, the NOV33a protein was found to have homology to the proteins shown in the BLASTP data in Table 33D.

TABLE 33D
Public BLASTP Results for NOV33a

| | | NOV33a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q95JN1 | HYPOTHETICAL 34.6 KDA | 85 . . . 359 | 244/315 (77%) | e−132 |
| | PROTEIN - Macaca fascicularis | 1 . . . 311 | 248/315 (78%) | |
| | (Crab eating macaque) | | | |
| | (Cynomolgus monkey), 312 aa. | | | |
| Q9D4M0 | 4931406B18RIK PROTEIN - Mus | 12 . . . 339 | 166/369 (44%) | 1e−79 |
| | musculus (Mouse), 367 aa. | 11 . . . 359 | 216/369 (57%) | |
| Q9D4Y7 | 4930538L19RIK PROTEIN - Mus | 52 . . . 312 | 133/267 (49%) | 1e−65 |
| | musculus (Mouse), 283 aa. | 1 . . . 247 | 173/267 (63%) | |
| AAD50978 | SIALIC ACID BINDING IG-LIKE | 25 . . . 232 | 69/226 (30%) | 1e−20 |
| | LECTIN-5 - Homo sapiens | 27 . . . 249 | 113/226 (49%) | |
| | (Human), 551 aa. | | | |
| O15389 | OB BINDING PROTEIN-2 | 25 . . . 232 | 69/226 (30%) | 1e−20 |
| | (SIGLEC5) - Homo sapiens | 27 . . . 249 | 113/226 (49%) | |
| | (Human), 551 aa. | | | |

### Example 34

The NOV34 clone was analyzed, and the nucleotide and encoded polypeptide sequences are shown in Table 34A.

TABLE 34A
NOV34 Sequence Analysis

| | |
|---|---|
| SEQ ID NO:99 | 1152 bp |

| | |
|---|---|
| NOV34a, | TGGGACCACGTGGGATGCTGCCGTGGCTTCTTGTCTTCTCTGCTCTGGGTCTCCAGG |
| CG109505-01 | |
| DNA Sequence | CCTGGGGTTCTTTCTCCTGGAGTGAGACCCAAGCCAGAGGCTTGTCCCAGAGGCTTAT |
| | GGACCTGTTTGTCAGCATCTCACAGTTCATTCACAAGGATACTCCCACCATCGTCTCC |
| | CGCAAGGAGTGGGGGGCAAGACCGCTCGCCTGCAGGGCCCTGCTGACCCTGCCTGTGG |
| | CCTACATCATCACAGACCAGCTCCCAGGGATGCAGTGCCAGCAGCAGAGCGTTTGCAG |
| | CCAGATGCTGCGGGGGTTGCAGTCCCATTCCGTCTACACCATAGGCTGGTGCGACGTG |
| | GCGTACAGCTTCCTGGTTGGGGATGATGGCAGGGTGTATGAAGGTGTTGGCTGGAACA |
| | TCCAAGGCTTGCACACCCAGGGCTACAACAACATTTCCCTGGGCATCGCCTTCTTTGG |
| | CAATAAGAAGGGTCACTCCCCCAGCCCTGCTGCCTTATCAGCTGCAGAGGGTCTGATC |
| | TCCTATGCCATCCAGAAGGGTCACCTGTCGCCCAGGTATATTCAGCCACTTCTTCTGA |
| | AAGAAGAGACCTGCCTGGACCCTCAACATCCAGTGATGCCCAGGAAGCAGCTTGCCCC |
| | GGCGTTGTCCCACGGTCTGTGTGGGGAGCCAGGGAGACCCTTATCAAAAATGAACCTC |
| | CCAGCCAAATATGTCATCATCATCCACACCGCTGGCACAAGCTGCACTGTATCCACAG |
| | ACTGCCAGACTGTCGTCCGAAACATACAGTCCTTTCACATGGACACACGGAACTTTTG |
| | TGACATTGGATATAGCTTCCTGGTGGGCCAGGATGGTGGCGTGTATGAAGGGGTTGGA |
| | TGGCACATCCAAGGCTCTCACACTTATGGATTCAACGATATTGCCCTAGGAATTGCCT |
| | TCATCGGCATCCCCTACTTTGTAGGTCCAAATGCTGCAGCGCTGGAGGCGGCCCAGGA |
| | CCTGATCCAGTGTGCCGTGGTTGAGGGGTACCTGACTCCAAACTACCTGCTGATGGGC |
| | CACAGTGACGTGGTCAACATCCTGTCCCCTGGGCAGGCTTTGTATAACATCATCAGCA |
| | CCTGGCCTCATTTCAAGCACTGAAGGAGGCCCCACTCCCTTTGAGACTGC |

| | |
|---|---|
| ORF Start: ATG at 16 | ORF Stop: TGA at 1123 |

| | | |
|---|---|---|
| SEQ ID NO: 100 | 369 aa | MW at 40411.0kd |

| | |
|---|---|
| NOV34a, | MLPWLLVFSALGLQAWGSFSWSETQARGLSQRLMDLFVSISQFIHKDTPTIVSRKEWG |
| CG109505-01 | |
| Protein Sequence | ARPLACRALLTLPVAYIITDQLPGMQCQQQSVCSQMLRGLQSHSVYTIGWCDVAYSFL |
| | VGDDGRVYEGVGWNIQGLHTQGYNNISLGIAFFGNKKGHSPSPAALSAAEGLISYAIQ |
| | KGHLSPRYIQPLLLKEETCLDPQHPVMPRKQLAPALSHGLCGEPGRPLSKMNLPAKYV |
| | IIIHTAGTSCTVSTDCQTVVRNIQSFHMDTRNFCDIGYSFLVGQDGGVYEGVGWHIQG |
| | SHTYGFNDIALGIAFIGIPYFVGPNAAALEAAQDLIQCAVVEGYLTPNYLLMGHSDVV |
| | NILSPGQALYNIISTWPHFKH |

Further analysis of the NOV34a protein yielded the following properties shown in Table 34B.

TABLE 34B
Protein Sequence Properties NOV34a

| | |
|---|---|
| PSort | 0.3894 probability located in outside; 0.1213 probability |
| analysis: | located in microbody (peroxisome); 0.1000 probability located |
| | in endoplasmic reticulum (membrane); |
| | 0.1000 probability located in endoplasmic reticulum (lumen) |
| SignalP | Cleavage site between residues 18 and 19 |
| analysis: | |

A search of the NOV34a protein against the Geneseq database, a proprietary database that contains sequences published in patents and patent publication, yielded several homologous proteins shown in Table 34C.

TABLE 34C
Geneseq Results for NOV34a

| | | NOV34a | Identities/ | |
|---|---|---|---|---|
| | | Residues/ | Similarities for | |
| Geneseq | Protein/Organism/Length [Patent | Match | the Matched | Expect |
| Identifier | #, Date] | Residues | Region | Value |
| AAY96963 | Wound healing tissue peptidoglycan | 1 . . . 369 | 252/369 (68%) | e−150 |
| | recognition protein-like protein - | 1 . . . 368 | 295/369 (79%) | |
| | Homo sapiens, 368 aa. | | | |
| | [WO200039327-A1, 06-JUL-2000] | | | |
| ABB53272 | Human polypeptide #12 - Homo | 1 . . . 369 | 236/369 (63%) | e−140 |
| | sapiens, 369 aa. [WO200181363- | 1 . . . 369 | 282/369 (75%) | |
| | A1, 01-NOV-2001] | | | |
| AAE00693 | Human full length granulocyte | 1 . . . 369 | 236/369 (63%) | e−140 |
| | peptide homolog Zgpa1 protein #2 - | 1 . . . 369 | 282/369 (75%) | |
| | Homo sapiens, 369 aa. | | | |
| | [WO200129224-A2, 26-APR-2001] | | | |
| AAE00692 | Human full length granulocyte | 1 . . . 369 | 235/375 (62%) | e−137 |
| | peptide homolog Zgpa1 protein #1 - | 1 . . . 375 | 282/375 (74%) | |
| | Homo sapiens, 375 aa. | | | |
| | [WO200129224-A2, 26-APR-2001] | | | |
| AAY76124 | Human secreted protein encoded by | 2 . . . 270 | 213/269 (79%) | e−118 |
| | gene 1 - Homo sapiens, 244 aa. | 4 . . . 242 | 215/269 (79%) | |
| | [WO9958660-A1, 18-NOV-1999] | | | |

In a BLAST search of public sequence databases, the NOV34a protein was found to have homology to the proteins shown in the BLASTP data in Table 34D.

TABLE 34D
Public BLASTP Results for NOV34a

| | | NOV34a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q96LB9 | PEPTIDOGLYCAN | 2 . . . 369 | 309/370 (83%) | e−175 |
| | RECOGNITION PROTEIN-I- | 4 . . . 341 | 311/370 (83%) | |
| | ALPHA PRECURSOR - Homo | | | |
| | sapiens (Human), 341 aa. | | | |
| Q9HD75 | HYPOTHETICAL 40.0 KDA | 1 . . . 369 | 252/369 (68%) | e−149 |
| | PROTEIN - Homo sapiens | 1 . . . 368 | 295/369 (79%) | |
| | (Human), 368 aa. | | | |
| CAC38715 | SEQUENCE 7 FROM PATENT | 1 . . . 369 | 236/369 (63%) | e−140 |
| | WO0129224 - Homo sapiens | 1 . . . 369 | 282/369 (75%) | |
| | (Human), 369 aa. | | | |
| Q96LB8 | PEPTIDOGLYCAN | 1 . . . 369 | 237/373 (63%) | e−138 |
| | RECOGNITION PROTEIN-I- | 1 . . . 373 | 282/373 (75%) | |
| | BETA PRECURSOR - Homo | | | |
| | sapiens (Human), 373 aa. | | | |
| CAC38714 | SEQUENCE 4 FROM PATENT | 1 . . . 369 | 235/375 (62%) | e−137 |
| | WO0129224 - Homo sapiens | 1 . . . 375 | 282/375 (74%) | |
| | (Human), 375 aa. | | | |

### Example 35

The NOV35 clone was analyzed, and the nucleotide and encoded polypeptide sequences are shown in Table 35A.

TABLE 35A
NOV35 Sequence Analysis

| | |
|---|---|
| SEQ ID NO: 101 | 3950 bp |

| | |
|---|---|
| NOV35a, | ATGCGCGGGGCGGGGGCGGCGGGGCTGCTGGCGCTGCTGCTGCTGCTGCTGCTGCTGC |
| CG109742-01 | |
| Protein Sequence | TGCTGGGCCTGGGCGGCAGGGTCGAGGGGGGGCCGGCCGGCGAGCGGGGCGCAGGCGG |
| | GGGCGGGGCGCTGGCCCGCGAGCGCTTCAAGGTGGTCTTTGCGCCGGTGATCTGCAAG |
| | CGGACCTGTCTCAAGGGCCAGTGTCGGGACAGTTGTCAGCAGGGCTCCAACATGACGC |
| | TCATCGGAGAGAACGGCCACAGCACAGACACGCTCACGGGCTCCGGCTTCCGCGTGGT |
| | GGTGTGCCCTCTCCCCTGCATGAATGGCGGCCAGTGCTCCTCGCGAAACCAGTGCCTG |
| | TGTCCCCCGGACTTCACTGGGCGCTTCTGCCAGGTGCCCGCAGGAGGAGCCGGTGGGG |
| | GTACCGGCGGCTCAGGCCCCGGCCTGAGCAGGACAGGGGCCCTGTCCACAGGGGCGCT |
| | GCCGCCCCTGGCTCCGGAGGGCGACTCTGTGGCCAGCAAGCACGCCATCTACGCCGTC |
| | CAGGTGATCGCTGACCCTCCTGGGCCCGGGGAGGGGCCTCCTGCCCAGCACGCAGCCT |
| | TCCTGGTGCCCCTAGGCCCGGGACAGATCTCAGCAGAAGTGCAGGCCCCGCCCCCCGT |
| | GGTGAATGTGCGCGTCCATCACCCGCCCGAGGCCTCAGTCCAGGTGCACCGCATTGAG |
| | AGCTCGAACGCCGAGAGCGCAGCCCCCTCCCAGCACCTGCTGCCGCACCCCAAGCCCT |
| | CGCACCCCCGGCCGCCCACCCAGAAGCCCCTGGGCCGCTGCTTTCAGGACACTCTGCC |
| | CAAGCAGCCGTGTGGCAGCAACCCCCTCCCCGGCCTCACCAAGCAGGAAGACTGCTGC |
| | GGTAGCATCGGCACTGCCTGGGGCCAGAGCAAGTGCCACAAGTGTCCCCAGCTGCAGT |
| | ACACAGGAGTGCAGAAGCCAGGGCCTGTACGTGGGGAAGTGGGCGCTGACTGTCCCCA |
| | GGGCTACAAGAGGCTTAACAGCACCCACTGCCAGGACATCAACGAGTGCGCAATGCCG |
| | GGCGTGTGTCGCCATGGTGACTGCCTCAACAACCCTGGCTCCTATCGCTGTGTCTGCC |
| | CACCTGGCCATAGTTTAGGCCCCTCCCGTACACAGTGCATTGCAGACAAACCGGAGGA |
| | GAAGAGCCTGTGTTTCCGCCTGGTGAGCCCTGAGCACCAGTGCCAGCACCCACTGACC |
| | ACCCGCCTGACCCGCCAGCTCTGCTGCTGCAGTGTCGGCAAGGCCTGGGGCGCGCGGT |
| | GTCAGCGCTGCCCAACAGATGGCACCGCTGCGTTCAAGGAGATCTGCCCAGCTGGGAA |
| | GGGATACCACATTCTCACCTCCCACCAGACGCTCACCATTCAGGGCGACAGTGACTTT |
| | TCCCTTTTCCTGCACCCTGACGGGCCACCCAAGCCCCAGCAGCTTCCGGAGAGCCCTA |
| | GCCAGGCTCCACCACCTGAGGACACAGAGGAAGAGAGAGGGGTGACCACGGACTCACC |
| | GGTGAGTGAGGAGAGGTCAGTGCAGCAGAGCCACCCAACTGCCACCACGACTCCTGCC |
| | CGGCCCTACCCCGAGCTGATCTCCCGTCCCTCGCCCCCGACCATGCGCTGGTTCCTGC |
| | CGGACTTGCCTCCTTCCCGCAGCGCCGTAGAGATCGCTCCCACTCAGGTCACAGAGAC |
| | TGATGAGTGCCGACTGAACCAGAACATCTGTGGCCACGGAGAGTGCGTGCCGGGCCCC |
| | CCTGACTACTCCTGCCACTGCAACCCCGGCTACCGGTCACATCCCCAGCACCGCTACT |
| | GCGTGGATGTGAACGAGTGCGAGGCAGAGCCCTGTGGCCCGGGGAGGGGCATCTGCAT |
| | GAACACCGGCGGCTCCTACAATTGCCACTGCAACCGCGGCTACCGCCTGCACGTGGGC |
| | GCCGGGGGGCGCTCGTGCGTGGACCTGAACGAATGCGCCAAGCCCCACCTGTGCGGCG |
| | ACGGCGGCTTCTGCATCAACTTTCCCGGTCACTACAAGTGCAACTGCTACCCCGGCTA |
| | CCGGCTCAAAGCCTCCCGGCCTCCTGTGTGCGAAGACATCGACGAGTGCCGGGACCCA |
| | AGCTCTTGCCCGGATGGCAAATGCGAGAACAAGCCCGGGAGCTTCAAGTGCATCGCCT |
| | GTCAGCCTGGCTGGTGCGAGAACCTCCCGGGCTCCTTCCGCTGCACCTGTGCCCAGGG |
| | CTACGCGCCCGCGCCCGACGGCCGCAGTTGCTTGGATGTGGACGAGTGTGAGGCTGGG |
| | GACGTGTGTGACAATGGCATCTGCAGCAACACGCCAGGATCTTTCCAGTGTCAGTGCC |
| | TCTCTGGCTACCATCTGTCCAGGGACCGGAGCCACTGCGAGGACATTGATGAGTGTGA |
| | CTTCCCTGCAGCCTGCATTGGGGGTGACTGCATCAATACCAATGGCTCCTACAGATGT |
| | CTTTGCCCCCAGGGGCATCGGCTGGTGGGTGGCAGGAAATGCCAAGACATAGATGAGT |
| | GCAGCCAGGACCCGAGCCTGTGCCTTCCCCATGGGGCCTGCAAGAACCTTCAGGGCTC |
| | CTATGTGTGTGTCTGCGATGAGGGCTTCACTCCCACCCAGGACCAGCACGGTTGTGAG |
| | GAGGTGGAGCAGCCCCACCACAAGAAGGAGTGCTACCTGAACTTCGATGACACAGTGT |
| | TCTGCGACAGCGTATTGGCCACCAACGTGACCCAGCAGGAGTGCTGCTGCTCTCTGGG |
| | GGCCGGCTGGGGCGACCACTGCGAAATCTACCCCTGCCCAGTCTACAGCTCAGCCGAG |
| | TTCCACAGCCTCTGCCCAGACGGAAAGGGCTACACCCAGGACAACAACATCGTCAACT |
| | ACGGCATCCCAGCCCACCGTGACATCGACGAGTGCATGTTGTTCGGGTCGGAGATTTG |
| | CAAGGAGGGCAAGTGCGTGAACACGCAGCCTGGCTACGAGTGCTACTGCAAGCAGGGC |
| | TTCTACTACGACGGGAACCTGCTGGAATGCGTGGACGTGGACGAGTGCCTGGACGAGT |
| | CCAACTGCCGGAACGGAGTGTGTGAGAACACGCGCGGCGGCTACCGCTGTGCCTGCAC |
| | GCCCCCTGCCGAGTACAGTCCCGCGCAGCGCCAGTGCCTGAGCCCGGAAGAGATGGAG |
| | CGTGCCCCGGAGCGGCGCGACGTGTGCTGGAGCCAGCGCGGAGAGGACGGCATGTGCG |
| | CTGGCCCCCTGGCCGGGCCTGCCCTCACCTTCGACGACTGCTGCTGCCGCCAGGGCCG |
| | CGGCTGGGGCGCCCAATGCCGACCGTGCCCGCCGCGCGGCGCGGGGTCCCATTGCCCG |
| | ACATCGCAGAGCGAGAGCAATTCCTTCTGGGACACAAGCCCCCTGCTGTTGGGGAAGC |
| | CCCCAAGAGATGAGGACAGTTCAGAGGAGGATTCAGACGAGTGTCGCTGCGTGAGTGG |
| | GACGCCTCCCGCGCCCGCTGCGTGGATATCGACGAGTGCCGAGAGCTGAACCAGCGCG |
| | GGCTGCTGTGCAAGAGCGAGCGCTGCGTGAACACCAGCGGCTCCTTCCGCTGCGTCTG |
| | CAAAGCCGGCTTCGCGCGCAGCCGCCCGCACGGGGCCTGCGTTCCCCAGCGCCGCCGC |
| | TGACGCCGCCGACGCCGCCCTCGGCCCAGACCTCGGTGATCACTGAGGGATTTCCGCG |
| | AGCTCGGCCTCACTTCTGCCCCGACTTGTGGCTCGGACCCAGGGACCTTCAGGGCCCG |
| | CAGACCCTCCCGGCGCCTTGAGACCCGAGGCGCCCCTACCGGCCCCCCTCCCCGGTTA |
| | GCGGGCGGTTGTAAGGTCTCCGGCGGGCGCTGCCTGCCTTCCTCCCAGAGGGTGTTTC |
| | AAAAAA |

| | |
|---|---|
| ORF Start: TG at 1 | ORF Stop: TGA at 3655 |

| | | |
|---|---|---|
| SEQ ID NO: 102 | 1218 aa | MW at 130645.3kD |

| | |
|---|---|
| NOV35a, | MRGAGAAGLLALLLLLLLLLLGLGGRVEGGPAGERGAGGGGALARERFKVVFAPVICK |
| CG109742-01 | |
| Protein Sequence | RTCLKGQCRDSCQQGSNMTLIGENGHSTDTLTGSGFRVVVCPLPCMNGGQCSSRNQCL |
| | CPPDFTGRFCQVPAGGAGGGTGGSGPGLSRTGALSTGALPPLAPEGDSVASKHAIYAV |
| | QVIADPPGPGEGPPAQHAAFLVPLGPGQISAEVQAPPPVVINRVHHPPEASVQVHRIE |
| | SSNAESAAPSQHLLPHPKPSHPRPPTQKPLGRCFQDTLPKQPCGSNPLPGLTKQEDCC |
| | GSIGTAWGQSKCHKCPQLQYTGVQKPGPVRGEVGADCPQGYKRLNSTHCQDINECAMP |
| | GVCRHGDCLNNPGSYRCVCPPGHSLGPSRTQCIADKPEEKSLCFRLVSPEHQCQHPLT |
| | TRLTRQLCCCSVGKAWGARCQRCPTDGTAAFKEICPAGKGYHILTSHQTLTIQGESDF |
| | SLFLHPDGPPKPQQLPESPSQAPPPEDTEEERGVTTDSPVSEERSVQQSHPTATTTPA |
| | RPYPELISRPSPPTMRWFLPDLPPSRSAVEIAPTQVTETDECRLNQNICGHGECVPGP |
| | PDYSCHCNPGYRSHPQHRYCVDVNECEAEPCGPGRGICMNTGGSYNCHCNRGYRLHVG |
| | AGGRSCVDLNECAKPHLCGDGGFCINFPGHYKCNCYPGYRLKASRPPVCEDIDECRDP |
| | SSCPDGKCENKPGSFKCIACQPGWCENLPGSFRCTCAQGYAPAPDGRSCLDVDECEAG |
| | DVCDNGICSNTPGSFQCQCLSGYHLSRDRSHCEDIDECDFPAACIGGDCINTNGSYRC |
| | LCPQGHRLVGGRKCQDIDECSQDPSLCLPHGACKNLQGSYVCVCDEGFTPTQDQHGCE |
| | EVEQPHHKKECYLNFDDTVFCDSVLATNVTQQECCCSLGAGWGDHCEIYPCPVYSSAE |
| | FHSLCPDGKGYTQDNNIVNYGIPAHRDIDECMLFGSEICKEGKCVNTQPGYECYCKQG |
| | FYYDGNLLECVDVDECLDESNCRNGVCENTRGGYRCACTPPAEYSPAQRQCLSPEEME |
| | RAPERRDVCWSQRGEDGMCAGPLAGPALTFDDCCCRQGRGWGAQCRPCPPRGAGSHCP |
| | TSQSESNSFWDTSPLLLGKPPRDEDSSEEDSDECPCVSGRCVPRPGGAVCECPGGFQL |
| | DASEARCVDIDECRELNQRGLLCKSERCVNTSGSFRCVCKAGFARSRPHGACVPQRRR |

| | |
|---|---|
| SEQ ID NO: 103 | 603 bp |

| | |
|---|---|
| NOV3 5b, | GGATCCCGCTTCAAGGTGGTCTTTGCGCCGGTGATCTGCAAGCGGACCTGTCTCAAGG |
| 207639410 DNA | |
| Sequence | GCCAGTGTCGGGACAGTTGTCAGCAGGGCTCCAACATGACGCTCATCGGAGAGAACGG |
| | CCACAGCACAGACACGCTCACGGGCTCCGGCTTCCGCGTGGTGGTGTGCCCTCTCCCC |
| | TGCATGAATGGCGGCCAGTGCTCCTCGCGAAACCAGTGCCTGTGTCCCCCGGACTTCA |
| | CTGGGCGCTTCTGCCAGGTGCCCGCAGGAGGAGCCGGTGGGGGTACCGGCGGCTCAGG |
| | CCCCGGCCTGAGCAGGACAGGGGCCCTGTCCACAGGGGCGCTGCCGCCCCTGGCTCCG |
| | GAGGGCGGCTCTGTGGCCAGCAAGCACGCCATCTACGCCGTCCAGGTGATCGCTGACC |
| | CTCCTGGGCCCGGGGAGGGGCCTCCTGCCCAGCACGCAGCCTTCCTGGTGCCCCTAGG |
| | CCCGGGACAGATCTCAGCAGAAGTGCAGGCCCCGCCCCCCGTGGTGAATGTGCGCGTC |
| | CATCACCCGCCCGAGGCCTCAGTCCAGGTGCACCGCATTGAGAGCTCGAACGCCGAGA |
| | GCGCAGCCCCCTCCCAGCTCGAG |

| | |
|---|---|
| ORE Start: at 1 | ORF Stop: end of sequence |

| | | |
|---|---|---|
| SEQ ID NO: 104 | 201 aa | MW at 20294.8kD |

| | |
|---|---|
| NOV35b, | GSRFKVVFAPVICKRTCLKGQCRDSCQQGSNMTLIGENGHSTDTLTGSGFRVVVCPLP |
| 207639410 | |
| Protein Sequence | CMNGGQCSSRNQCLCPPDFTGRFCQVPAGGAGGGTGGSGPGLSRTGALSTGALPPLAP |
| | EGGSVASKRAIYAVQVIADPPGPGEGPPAQHAAFLVPLGPGQISAEVQAPPPVVNVRV |
| | HHPPEASVQVHRIESSNAESAAPSQLE |

| | |
|---|---|
| SEQ ID NO: 105 | 603 bp |

| | |
|---|---|
| NOV35c, | GGATCCCGCTTCAAGGTGGTCTTTGCGCCGGTGATCTGCAAGCGGACCTGTCTCAAGG |
| 207639427 DNA | |
| Sequence | GCCAGTGTCGGGACAGTTGTCAGCAGGGCTCCAACATGACGCTCATCGGAGAGAACGG |
| | CCACAGCACAGACACGCTCACGGGCTCCGGCTTCCGCGTGGTGGTGTGCCCTCTCCCC |
| | TGCATGAATGGCGGCCAGTGCTCCTCGCGAAACCAGTGCCTGTGTCCCCCGGACTTCA |
| | CTGGGCGCTTCTGCCAGGTGCCCGCAGGAGGAGCCGGTGGGGGTACCGGCGGCTCAGG |
| | CCCCGGCCTGAGCAGGACAGGGGCCCTGTCCACAGGGGCGCTGCCGCCCCTGGCTCCG |
| | GAGGGCGACTCTGTGGCCAGCACGCACGCCATCTACGCCGTCCAGGTGATCGCTGACC |
| | CTCCTGGGCCCGGGGAGGGGCCTCCTGCCCAGCACGCAGCCTTCCTGGTGCCCCTAGG |
| | CCCGGGACAGATCTCAACAGAAGTGCAGGCCCCGCCCCCCGTGGTGAATGTGCGCGTC |
| | CATCACCCGCCCGAGGCCTCAGTCCAGGTGCACCGCATTGAGAGCTCGAACGCCGAGA |
| | GCGCAGCCCCCTCCCAGCTCGAG |

| | |
|---|---|
| ORE Start: at 1 | ORE Stop: end of Sequence |

| | | |
|---|---|---|
| SEQ ID NO: 106 | 201 aa | MW at 20382.9kD |

| | |
|---|---|
| NOV35c, | GSRFKVVFAPVICKRTCLKGQCRDSCQQGSNMTLIGENGHSTDTLTGSGFRVVVCPLP |
| 207639427 | |
| Protein Sequence | CMNGGQCSSRNQCLCPPDFTGRFCQVPAGGAGGGTGGSGPGLSRTGALSTGALPPLAP |
| | EGDSVASKHAIYAVQVIADPPGPGEGPPAQHAAFLVPLGPGQISTEVQAPPPVVNVRV |
| | HHPPEASVQVHRIESSNAESAAPSQLE |

| | |
|---|---|
| SEQ ID NO: 107 | 603 bp |

| | |
|---|---|
| NOV35d, | GGATCCCGCTTCAAGGTGGTCTTTGCGCCGGTGATCTGCAAGCGGACCTGTCTCAAGG |
| 2O7639438 DNA | |
| Sequence | GCCAGTGTCGGGACAGTTGTCCGCAGGGCTCCAACATGACGCTCATCGGAGAGAACGG |
| | CCACAGCACAGACACGCTCACGGGCTCCGGCTTCCGCGTGGTGGTGTGCCCTCTCCCC |
| | TGCACGAATGGCGGCCAGTGCTCCTCGCGAAACCAGTGCCTGTGTCCCCCGGACTTCA |
| | CTGGGCGCTTCTGCCAGGTGCCCGCAGGAGGAGCCGGTGGGGGTACCGGCGGCTCAGG |
| | CCCCGGCCTGAGCAGGACAGGGGCCCTGTCCACAGGGGCGCTGCCGCCCCTGGCTCCG |
| | GAGGGCGACTCTGTGGCCAGCAAGCACGCCATCTACGCCGTCCAGGTGATCGCTGACC |
| | CTCCTGGGCCCGGGGAGGGGCCTCCTGCCCAGCACGCAGCCTTCCTGGTGCCCCTAGG |
| | CCCGGGACAGATCTCAGCAGAAGTGCAGGCCCCGCCCCCCGTGGTGAATGTGCGCGTC |
| | CATCACCCGCCCGAGGCCTCAGTCCAGGTGCACCGCATTGAGAGCTCGAACGCCGAGA |
| | GCGCAGCCCCCTCCCAGCTCGAG |

| | |
|---|---|
| ORF Start: at 1 | ORF Stop: end of sequence |

| | | |
|---|---|---|
| SEQ ID NO: 108 | 201 aa | MW at 20291.7kD |

| | |
|---|---|
| NOV35d, | GSRFKVVFAPVICKRTCLKGQCRDSCPQGSNMTLIGENGHSTDTLTGSGFRVVVCPLP |
| 207639438 | |
| Protein Sequence | CTNGGQCSSRNQCLCPPDFTGRFCQVPAGGAGGGTGGSGPGLSRTGALSTGALPPLAP |
| | EGDSVASKHAIYAVQVIADPPGPGEGPPAQHAAFLVPLGPGQISAEVQAPPPVVNVRV |
| | HHPPEASVQVHRIESSNAESAAPSQLE |

| | |
|---|---|
| SEQ ID NO: 109 | 603 bp |

| | |
|---|---|
| NOV35e, | GGATCCCGCTTCAAGGTGGTCTTTGCGCCGGTGATCTGCAAGCGGACCTGTCTCGAGG |
| 207639448 DNA | |
| Sequence | GCCAGTGTCGGGACAGTTGTCAGCAGGGCTCCAACATGACGCTCATCGGAGAGAACGG |
| | CCACAGCACAGACACGCTCACGGGCTCCGGCTTCCGCGTGGTGGTGTGCCCTCTCCCC |
| | TGCATGAATGGCGGCCAGTGCTCCTCGCGAAACCAGTGCCTGTGTCCCCCGGACTTCA |
| | CTGGGCGCTTCTGCCAGGTGCCCGCAGGAGGAGCCGGTGGGGGTACCGGCGGCTCAGG |
| | CCCCGGCCTGAGCAGGACAGGGGCCCTGTCCACAGGGGCGCTGCCGCCCCTGGCTCCG |
| | GAGGGCGACTCTGTGGCCAGCAAGCACGCCATCTACGCCGTCCAGGTGATCGCTGACC |
| | CTCCTGGGCCCOGGGAGGGGCCTCCTGCCCAGCACGCAGCCTTCCTGGTGCCCCTAGG |
| | CCCGGGACAGATCTCAGCAGAAGTGCAGGCCCCGCCCCCCGTGGTGAATGTGCGCGTC |
| | CATCACCCGCCCGAGGCCTCAGTCCAGGTGCACCGCATTGAGAGCTCGAACGCCGAGA |
| | GCGCAGCCCCCTCCCAGCTCGAG |

| | |
|---|---|
| ORF Start: at 1 | ORF Stop: end of sequence |

| | | |
|---|---|---|
| SEQ ID NO: 110 | 201 aa | MW at 20353.8kD |

| | |
|---|---|
| NOV35e, | GSRFKVVFAPVICKRTCLEGQCRDSCQQGSNMTLIGENGHSTDTLTGSGRFVVVCPLP |
| 207639448 | |
| Protein Sequence | CMNGGQCSSRNQCLCPPDFTGRFCQVPAGGAGGGTGGSGPGLSRTGALSTGALPPLAP |
| | EGDSVASKHAIYAVQVIADPPGPGEGPPAQHAAFLVPLGPGQISAEVQAPPPVVNVRV |
| | HHPPEASVQVHRIESSNAESAAPSQLE |

Sequence comparison of the above protein sequences yields the following sequence relationships shown in Table 35B.

TABLE 35B
Comparison of NOV35a against NOV35b through NOV35e.

| | NOV35a Residues/ | Identities/Similarities |
|---|---|---|
| Protein Sequence | Match Residues | for the Matched Region |
| NOV35b | 47 . . . 243 | 154/197 (78%) |
| | 3 . . . 199 | 154/197 (78%) |
| NOV35c | 47 . . . 243 | 154/197 (78%) |
| | 3 . . . 199 | 154/197 (78%) |
| NOV35d | 47 . . . 243 | 153/197 (77%) |
| | 3 . . . 199 | 153/197 (77%) |
| NOV35e | 47 . . . 243 | 154/197 (78%) |
| | 3 . . . 199 | 155/197 (78%) |

Further analysis of the NOV35a protein yielded the following properties shown in Table 35C.

TABLE 35C
Protein Sequence Properties NOV35a

| | |
|---|---|
| PSort | 0.8200 probability located in outside; 0.1900 probability |
| analysis: | located in lysosome (lumen); 0.1000 probability located in |
| | endoplasmic reticulum (membrane); 0.1000 probability |
| | located in endoplasmic reticulum (lumen) |
| SignalP | Cleavage site between residues 30 and 31 |
| analysis: | |

A search of the NOV35a protein against the Geneseq database, a proprietary database that contains sequences published in patents and patent publication, yielded several homologous proteins shown in Table 35D.

TABLE 35D
Geneseq Results for NOV35a

| | | NOV35a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAB61419 | Human TANGO 275 protein - | 1 . . . 1218 | 1217/1289 (94%) | 0.0 |
| | Homo sapiens, 1289 aa. | 1 . . . 1289 | 1217/1289 (94%) | |
| | [WO200100672-A1, 04-JAN-2001] | | | |
| AAY70551 | Human latent transforming growth | 35 . . . 1218 | 1183/1208 (97%) | 0.0 |
| | factor-beta binding protein 3 (I) - | 1 . . . 1208 | 1183/1208 (97%) | |
| | Homo sapiens, 1208 aa. | | | |
| | [WO200012551-A1, 09-MAR-2000] | | | |
| AAY70554 | Human latent transforming growth | 35 . . . 1218 | 1183/1257 (94%) | 0.0 |
| | factor-beta binding protein 3 (III) - | 1 . . . 1257 | 1183/1257 (94%) | |
| | Homo sapiens, 1257 aa. | | | |
| | [WO200012551-A1, 09-MAR-2000] | | | |
| AAB61483 | Human TANGO 300 extracellular | 10 . . . 1213 | 1056/1230 (85%) | 0.0 |
| | domain - Homo sapiens, 1251 aa. | 6 . . . 1229 | 1078/1230 (86%) | |
| | [WO200100672-A1, 04-JAN-2001] | | | |
| AAR79475 | Mouse LTBP-3 - Mus sp, 1251 aa. | 10 . . . 1213 | 1056/1230 (85%) | 0.0 |
| | [WO9522611-A2, 24-AUG-1995] | 6 . . . 1229 | 1078/1230 (86%) | |

In a BLAST search of public sequence databases, the NOV35a protein was found to have homology to the proteins shown in the BLASTP data in Table 35E.

TABLE 35E
Public BLASTP Results for NOV35a

| | | NOV35a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9NS15 | LATENT TRANSFORMING | 1 . . . 1218 | 1218/1242 (98%) | 0.0 |
| | GROWTH FACTOR BETA | 15 . . . 1256 | 1218/1242 (98%) | |
| | BINDING PROTEIN 3 - Homo | | | |
| | sapiens (Human), 1256 aa. | | | |
| Q9H7K2 | FLJ00070 PROTEIN - Homo | 1 . . . 1218 | 1217/1289 (94%) | 0.0 |
| | sapiens (Human), 1382 aa | 95 . . . 1382 | 1217/1289 (94%) | |
| | (fragment). | | | |
| A57293 | latent transforming growth factor | 10 . . . 1213 | 1056/1230 (85%) | 0.0 |
| | beta-binding protein 3 precursor - | 6 . . . 1229 | 1078/1230 (86%) | |
| | mouse, 1251 aa. | | | |
| Q61810 | LATENT TRANSFORMING | 1 . . . 1213 | 1054/1240 (85%) | 0.0 |
| | GROWTH FACTOR-BETA | 1 . . . 1231 | 1074/1240 (86%) | |
| | BINDING PROTEIN - Mus | | | |
| | musculus (Mouse), 1253 aa. | | | |
| Q96HB9 | SIMILAR TO LATENT | 500 . . . 1218 | 719/743 (96%) | 0.0 |
| | TRANSFORMING GROWTH | 4 . . . 746 | 719/743 (96%) | |
| | FACTOR BETA BINDING | | | |
| | PROTEIN 3 - Homo sapiens | | | |
| | (Human), 746 aa (fragment). | | | |

PFam analysis predicts that the NOV35a protein contains the domains shown in the Table 35F.

TABLE 35F
Domain Analysis of NOV35a

| | | Identities/ | |
|---|---|---|---|
| | NOV35a | Similarities | Expect |
| Pfam Domain | Match Region | for the Matched Region | Value |
| EGF | 99 . . . 126 | 14/47 (30%) | 0.00034 |
| | | 23/47 (49%) | |
| TB | 273 . . . 316 | 20/48 (42%) | 0.013 |
| | | 32/48 (67%) | |
| EGF | 345 . . . 380 | 16/47 (34%) | 0.00073 |
| | | 28/47 (60%) | |
| TB | 399 . . . 440 | 23/47 (49%) | 3.5e−17 |
| | | 35/47 (74%) | |
| EGF | 564 . . . 600 | 14/47 (30%) | 0.0033 |
| | | 28/47 (60%) | |
| EGF | 606 . . . 644 | 17/48 (35%) | 0.66 |
| | | 28/48 (58%) | |
| EGF | 716 . . . 745 | 12/47 (26%) | 0.0073 |
| | | 26/47 (55%) | |
| EGF | 751 . . . 786 | 16/47 (34%) | 3e−07 |
| | | 28/47 (60%) | |
| EGF | 792 . . . 826 | 14/47 (30%) | 0.069 |
| | | 26/47 (55%) | |
| EGF | 832 . . . 869 | 11/47 (23%) | 8.6e−05 |
| | | 26/47 (55%) | |
| TB | 889 . . . 932 | 21/47 (45%) | 6.7e−14 |
| | | 33/47 (70%) | |
| EGF | 959 . . . 996 | 14/47 (30%) | 0.0034 |
| | | 28/47 (60%) | |
| EGF | 1002 . . . 1037 | 15/47 (32%) | 2.2e−05 |
| | | 29/47 (62%) | |
| TB | 1061 . . . 1106 | 18/48 (38%) | 0.0014 |
| | | 33/48 (69%) | |
| EGF | 1173 . . . 1208 | 12/49 (24%) | 0.32 |
| | | 26/49 (53%) | |

### Example 36

The NOV36 clone was analyzed, and the nucleotide and encoded polypeptide sequences are shown in Table 36A.

TABLE 36A
NOV36 Sequence Analysis

| | |
|---|---|
| SEQ ID NO: 111 | 1846 bp |

| | |
|---|---|
| NOV36a, | ATCATGCACTCCCAGAGACCTCCACTTGCACTCCTTGAGGGCTCCACCCTCGATAGAA |
| CG109844-01 | |
| DNA Sequence | AAGGAGAAATCGCAGCTTCACTCGTCTCTAGGCTGTGGAAAGTCTCCAATTCAACTCT |
| | GTTCCAAATGATGCTGGTCACTGTTTTGTTGGCTACCATTCTTGGTGACTGTGGTCCT |
| | CCACCTGAGTTACCATTTGCTTTTCCAATAAATCCGTTGTATGATACTGAATTCAAAA |
| | CTGGAACTACTCTGAAGTACACCTGCCACCCTGGGCATGGTAAAATCAATTCAAGTCG |
| | ACTGATTTGTGATGCCAAAGACTCGTGGAACTATAGTATCTTTTGTGCAAGTAAGCGA |
| | TGCAGAAATCCAGAATTAATCAATGGGATAGTGGAAGTTAAAAAAGATCTTCTCCTTG |
| | GTTCAACCATAAAATTCAGCTGCTCAGAGGGGTTTTTCTTAATTGGCTCAACCACCAG |
| | TCATTGTCAGATCCAAGGTAAAGGAGTTGATTGGAGTGATCCTCTCCCAGAAACCTCA |
| | GTTGCCAAGTGCGAGCCCCCTCCAGACATCAGGAATGGGAAGCACAGCGGTGGAGATC |
| | AAGAATTCTACACATATGCCTCCTCTGTCACCTACAGCTGCAACCCCTACTTCTCACT |
| | CATAGGCAACGTCTCCATCTCCTGCACCGTGGAGAATGAAACAATAGGTGTCTGGAGC |
| | CCAAACCCTCCTATCTGTGAAGAAATTGTCTGTCGTCGACCACAGATTCCAAAGGCAA |
| | TCTTTGTTTCTGGATTTGGACCCCTCTATACTTACAAAGACTCTATTATGGTTAACTG |
| | TGAGGAAGGTTATATCCTCAGAGGCAGCAGTTTAATCTATTGTGAAACGAATAATGAG |
| | TGGTATCCTTCTGTTCCCTCTTGCAGAGTGAATGGTTGCACTGTCCTACCGGACATTT |
| | CCTATGCTTCCTGGGAGAGAAATGACTACAACCTAAGTGATCACGAAATATTTGAAAT |
| | TGGAACTGAGTTGAAATATCTATGCAAACCTGGCTATAGACCTGTTTTAGATGAGCCT |
| | CTGACTGTGACTTGTCAGGAAAATTTCACATGGACATCTTCCAATGAGTGTGAGAGTG |
| | TATGTTGCCCAACACCAGATCTGGAGAATATCAGAATCATAAATGAAAGGAGGTATTT |
| | CACTGGTAGATGTGTCTATGCCTATGGAGACTATATTTCATATATGTGTGATGAAGGC |
| | TATTACCCTATTTCTGTTGACGGGGAGAGTTCCTGCCACACAGATGGCACATGGAAGC |
| | CTAAAATGCCAGCATGTGAGCCAGGTTGCAGTTTTGCCCCTAGTTTTGCCCATGGGCA |
| | TCCTAAACAAGTTAATTTATGCAACTGTTTCAAAAATGAGGCTGTATATAAATGTGAT |
| | GAAGGCTACACTGTGATCGGACAGGTGAAACTCACCTGCATTTCTTCCTGCTGGTCAT |
| | CTCCAGCCCCTCAATGTAAAAGTCTGTGTCTGAAACCAGAAATAGTGAATGGAAGGCT |
| | GTCTGTGGATAAGGATCAGTATGTTGAGTCTGAAAATGTTACCATTGAATGTGATTCT |
| | GGCTATGGTGTGGTTGGTCTCAAAAGTATCACTTGCTCAGAGAAGAGAACCTGGTACC |
| | CAGAAGTGCCCAGGTGTGAGTGGGAGGCACCTGAAGGTTGTGAGCAAGTGCTCACAGG |
| | CAGAAAACTCATGCAGTGTCTCCCAAGCCCAGAGGATGTGAAAGTGGCCCTGGAGGTG |
| | TATAAGCTGTCTCTGGAGATAAAACAACTTGAAAAAGAGAGAGACAAATTGATGAACA |
| | CCCATCAGAAATTTTCTGAAAAAGAGGAATGAAGGACTTATTTTTCCC |

| | |
|---|---|
| ORF Start: ATG at 4 | ORF Stop: TGA at 1828 |

| | | |
|---|---|---|
| SEQ ID NO: 112 | 608 aa | MW at 68085.6kD |

| | |
|---|---|
| NOV36a | NHSQRPPLALLEGSTLDRKGEIAASLVSRLWKVSNSTLFQMMLVTVLLATILGDCGPP |
| CG109844-01 | |
| Protein Sequence | PELPFAFPINPLYDTEFKTGTTLKYTCHPGHGKINSSRLICDAKDSWNYSIFCASKRC |
| | RNPELINGIVEVKKDLLLGSTIKFSCSEGFFLIGSTTSHCQIQGKGVDWSDPLPETSV |
| | AKCEPPPDIRNGKHSGGDQEFYTYASSVTYSCNPYFSLIGNVSISCTVENETIGVWSP |
| | NPPICEEIVCRRPQIPKAIFVSGFGPLYTYKDSIMVNCEEGYILRGSSLIYCETNNEW |
| | YPSVPSCRVNGCTVLPDISYASWERNDYNLSDHEIFEIGTELKYLCKPGYRPVLDEPL |
| | TVTCQENLTWTSSNECESVCCPTPDLENIRIINERRYFTGRCVYAYGDYISYMCDEGY |
| | YPISVDGESSCHTDGTWKPKMPACEPGCSFAPSFAHGHPKQVNLCNCFKUEAVYKCDE |
| | GYTVIGQVKLTCISSCWSSPAPQCKSLCLKPEIVNGRLSVDKDQYVESENVTIECDSG |
| | YGVVGLKSITCSEKRTWYPEVPRCEWEAPEGCEQVLTGRKLMQCLPSPEDVKVALEVY |
| | KLSLEIKQLEKERDKLMNTHQKFSEKEE |

Further analysis of the NOV36a protein yielded the following properties shown in Table 36B.

TABLE 36B
Protein Sequence Properties NOV36a

| | |
|---|---|
| PSort | 0.7900 probability located in plasma membrane; |
| analysis: | |
| | 0.3000 probability located in Golgi body; 0.2000 probability |
| | located in endoplasmic reticulum (membrane) |
| SignalP | Cleavage site between residues 54 and 55 |
| analysis: | |

A search of the NOV36a protein against the Geneseq database, a proprietary database that contains sequences published in patents and patent publication, yielded several homologous proteins shown in Table 36C.

TABLE 36C
Geneseq Results for NOV36a

| | | NOV36a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAR13490 | Human C4 binding protein - Homo | 22 . . . 594 | 341/577 (59%) | 0.0 |
| | sapiens, 581 aa. [WO9111461-A, | 1 . . . 570 | 428/577 (74%) | |
| | 08-AUG-1991] | | | |
| AAW39924 | Amino acid sequence of a mouse | 30 . . . 602 | 315/575 (54%) | 0.0 |
| | sperm protein designated sp56 - | 9 . . . 565 | 403/575 (69%) | |
| | Mus sp, 579 aa. [WO9800440-A1, | | | |
| | 08-JAN-1998] | | | |
| AAB43640 | Human cancer associated protein | 390 . . . 594 | 136/205 (66%) | 3e−80 |
| | sequence SEQ ID NO: 1085 - | 10 . . . 209 | 162/205 (78%) | |
| | Homo sapiens, 220 aa. | | | |
| | [WO200055350-A1, 21-SEP-2000] | | | |
| AAM50797 | Human C3B/C4B receptor CR1 | 48 . . . 539 | 158/532 (29%) | 2e−59 |
| | (complement receptor type 1) - | 1389 . . . 1897 | 238/532 (44%) | |
| | Homo sapiens, 2039 aa. | | | |
| | [US6316604-B1, 13-NOV-2001] | | | |
| ABG00287 | Novel human diagnostic protein | 48 . . . 539 | 158/532 (29%) | 2e−59 |
| | #278 - Homo sapiens, 2039 aa. | 1389 . . . 1897 | 238/532 (44%) | |
| | [WO200175067-A2, 11-OCT-2001] | | | |

In a BLAST search of public sequence databases, the NOV36a protein was found to have homology to the proteins shown in the BLASTP data in Table 36D.

TABLE 36D
Public BLASTP Results for NOV36a

| | | NOV36a | Identities/ | |
|---|---|---|---|---|
| Protein | | Residues/ | Similarities for | |
| Accession | | Match | the Matched | |
| Number | Protein/Organism/Length | Residues | Portion | Expect Value |
| P04003 | C4b-binding protein alpha chain | 1 . . . 594 | 347/598 (58%) | 0.0 |
| | precursor (C4bp) (Proline-rich | 1 . . . 586 | 436/598 (72%) | |
| | protein) (PRP) - Homo sapiens | | | |
| | (Human), 597 aa. | | | |
| S53711 | C4BP alpha chain precursor - | 1 . . . 595 | 330/599 (55%) | 0.0 |
| | rabbit, 597 aa. | 1 . . . 587 | 414/599 (69%) | |
| Q60736 | SPERM FERTILIZATION | 30 . . . 602 | 315/575 (54%) | 0.0 |
| | PROTEIN SP56 PRECURSOR - | 9 . . . 565 | 403/575 (69%) | |
| | Mus musculus (Mouse), 579 aa. | | | |
| Q28065 | C4b-binding protein alpha chain | 1 . . . 595 | 327/601 (54%) | 0.0 |
| | precursor (C4bp) - Bos taurus | 1 . . . 590 | 416/601 (68%) | |
| | (Bovine), 610 aa. | | | |
| Q63514 | C4b-binding protein alpha chain | 41 . . . 595 | 278/558 (49%) | e−174 |
| | precursor (C4bp) - Rattus | 1 . . . 550 | 385/558 (68%) | |
| | norvegicus (Rat), 558 aa. | | | |

PFam analysis predicts that the NOV36a protein contains the domains shown in the Table 36E.

TABLE 36E
Domain Analysis of NOV36a

| | | Identities/ | |
|---|---|---|---|
| | NOV36a | Similarities | Expect |
| Pfam Domain | Match Region | for the Matched Region | Value |
| sushi | 55 . . . 111 | 14/66 (21%) | 7.2e−10 |
| | | 41/66 (62%) | |
| sushi | 116 . . . 172 | 21/63 (33%) | 2.2e−08 |
| | | 41/63 (65%) | |
| sushi | 177 . . . 237 | 27/67 (40%) | 9.2e−17 |
| | | 49/67 (73%) | |
| sushi | 242 . . . 297 | 21/64 (33%) | 6.6e−12 |
| | | 40/64 (62%) | |
| sushi | 302 . . . 364 | 17/72 (24%) | 9.9e−05 |
| | | 46/72 (64%) | |
| sushi | 369 . . . 430 | 23/70 (33%) | 4.1e−11 |
| | | 46/70 (66%) | |
| sushi | 434 . . . 488 | 16/64 (25%) | 2.3e−06 |
| | | 35/64 (55%) | |
| sushi | 492 . . . 546 | 24/63 (38%) | 3.7e−11 |
| | | 37/63 (59%) | |

### Example 37

The NOV37 clone was analyzed, and the nucleotide and encoded polypeptide sequences are shown in Table 37A.

TABLE 37A
NOV37 Sequence Analysis

| | |
|---|---|
| SEQ ID NO: 113 | 2974 bp |

| | |
|---|---|
| NOV37a, | CGGGGACTCGGAGGTACTGGGCGCGCGCGGCTCCGGCTCGGGACGCCTCGGGACGCCT |
| CG110014-02 | |
| DNA Sequence | CGGGGTCGGGTTCCGGTTGCGGCTGCTGCTGCGGCGCCCGCGCTTCCGTAGCGTTCCG |
| | CCTCCTGTGCCCGCCGCGGAGCAAGTCTGCGCGCCCGCCGTGCGCCCCTAAGCTCCTT |
| | TTACCTGAGCCCGCCGCGATGGGAGCTGCGCGGGGATCCCCGGCCAGACCCCGCCGGT |
| | TGCCTCTGCTCAGCGTCCTGCTGCTGCCGCTGCTGGGCGGTACCCAGACAGCCATTGT |
| | CTTCATCAAGCAGCCGTCCTCCCAGGATGCACTGCAGGGGCGCCGGGCGCTGCTTCGC |
| | TGTGAGGTTGAGGCTCCGGGCCCGGTACATGTGTACTGGCTGCTCGATGGGGCCCCTG |
| | TCCAGGACACGGAGCGGCGTTTCGCCCAGGGCAGCAGCCTGAGCTTTGCAGCTGTGGA |
| | CCCGCTGCAGGACTCTGGCACCTTCCAGTGTGTGGCTCGGGATGATGTCACTGGAGAA |
| | GAAGCCCGCAGTGCCAACGCCTCCTTCAACATCAAATGGATTGAGGCAGGTCCTGTGG |
| | TCCTGAAGCATCCAGCCTCGGAAGCTGAGATCCAGCCACAGACCCAGGTCAAACTTCG |
| | TTGCCACATTGATGGGCACCCTCGGCCCACCTACCAATGGTTCCGAGATGGGACCCCC |
| | CTTTCTGATGGTCAGAGCAACCACACAGTCAGCAGCAAGGAGCGGAACCTGACGCTCC |
| | GGCCAGCTGGTCCTGAGCATAGTGGGCTGTATTCCTGCTGCGCCCACAGTGCTTTTGG |
| | CCAGGCTTGCAGCAGCCAGAACTTCACCTTGAGCATTGCTGATGAAAGCTTTGCCAGG |
| | GTGGTGCTGGCACCCCAGGACGTGGTAGTAGCGAGGTATGAGGAGGCCATGTTCCATT |
| | GCCAGTTCTCAGCCCAGCCACCCCCGAGCCTGCAGTGGCTCTTTGAGGATGAGACTCC |
| | CATCACTAACCGCAGTCGCCCCCCACACCTCCGCAGAGCCACAGTGTTTGCCAACGGG |
| | TCTCTGCTGCTGACCCAGGTCCGGCCACGCAATGCAGGGATCTACCGCTGCATTGGCC |
| | AGGGGCAGAGGGGCCCACCCATCATCCTGGAAGCCACACTTCACCTAGCAGAGATTGA |
| | AGACATGCCGCTATTTGAGCCACGGGTGTTTACAGCTGGCAGCGAGGAGCGTGTGACC |
| | TGCCTTCCCCCCAAGGGTCTGCCAGAGCCCAGCGTGTGGTGGGAGCACGCGGGAGTCC |
| | GGCTGCCCACCCATGGCAGGGTCTACCAGAASGGCCACGAGCTGGTGTTGGCCAATAT |
| | TGCTGAAAGTGATGCTGGTGTCTACACCTGCCACGCGGCCAACCTGGCTGGTCAGCGG |
| | AGACAGGATGCCAACATCACTGTGGCCACTGTGCCCTCCTGGCTGAAGAAGCCCCAAG |
| | ACAGCCAGCTGGAGGAGGGCAAACCCGGCTACTTGGATTGCCTGACCCAGGCCACACC |
| | AAAACCTACAGTTGTCTGGTACAGAAACCAGATGCTCATCTCAGACGACTCACGGTTC |
| | GAGGTCTTCAAGAATGGGACCTTGCGCATCAACAGCGTGGAGGTGTATGATGGGACAT |
| | GGTACCGTTGTATGAGCAGCACCCCAGCCGGCAGCATCGAGGCGCAAGCCCGTGTCCA |
| | AGTGCTGGAAAAGCTCAAGTTCACACCACCACCCCAGCCACAGCAGTGCATGGAGTTT |
| | GACAAGGAGGCCACGGTGCCCTGTTCAGCCACAGGCCGAGAGAAGCCCACTATTAAGT |
| | GGGAACGGGCAGATGGGAGCAGCCTCCCAGAGTGGGTGACAGACAACGCTGGGACCCT |
| | GCATTTTGCCCGGGTGACTCGAGATGACGCTGGCAACTACACTTGCATTGCCTCCAAC |
| | GGGCCGCAGGGCCAGATTCGTGCCCATGTCCAGCTCACTGTGGCAGTTTTTATCACCT |
| | TCAAAGTGGAACCAGAGCGTACGACTGTGTACCCTTCCAAACTCTATCGGCTGATGCA |
| | GCGCTGCTGGGCCCTCAGCCCCAAGGACCGGCCCTCCTTCAGTGAGATTGCCAGCGCC |
| | CTGGGAGACAGCACCGTGGACAGCAAGCCGTGAGGAGGGAGCCCGCTCAGGATGGCCT |
| | GGGCAGGGGAGGACATCTCTAGAGGGAAGCTCACAGCATGATGGGCAAGATCCCTGTC |
| | CTCCTGGGCCCTGAGGCCCCTGCCCTAGTGCAACAGGCATTGCTGAGGTCTGAGCAGG |
| | GCCTGGCCTTTCCTCCTCTTCCTCACCCTCATCCTTTGGGAGGCTGACTTGGACCCAA |
| | ACTGGGCGACTAGGGCTTTGAGCTGGGCAGTTTTCCCTGCCACCTCTTCCTCTATCAG |
| | GGACAGTGTGGGTGCCACAGGTAACCCCAATTTCTGGCCTTCAACTTCTCCCCTTGAC |
| | CGGGTCCAACTCTGCCACTCATCTGCCAACTTTGCCTGGGGAGGGCTAGGCTTGGGAT |
| | GAGCTGGGTTTGTGGGGAGTTCCTTAATATTCTCAAGTTCTGGGCACACAGGGTTAAT |
| | GAGTCTCTTGGCCCACTGGTCCCACTTGGGGGTCTAGACCAGGATTATAGAGGACACA |
| | GCAAGTGAGTCCTCCCCACTCTGGGCTTGTGCACACTGACCCAGACCCACGGTCTTCC |
| | CCACCCTTCTCTCCTTTCCTCATCCTAAGTGCCTGGCAGATGAAGGAGTTTTCAGGAG |
| | CTTTTGACACTATATAAACCGCCCTTTTTGTATGCACCACGGGCGGCTTTTATATGTA |
| | ATTGCAGCGTGGGGTGGGTGGGCATGGGAGGTAGGGGTGGGCCCTGGAGATGAGGAGG |
| | GTGGGCCATCCTTACCCCACACTTTTATTGTTGTCGTTTTTTGTGTGTTTGTGTTTTT |
| | TTGTTTTTGTTTTTGTTTTTACACTCGCTGCTCTCAATAAATAAGCCTTTTTAAAAAA |
| | AAAAAAAAAAAAAAAA |

| | |
|---|---|
| ORF Start: ATG at 193 | ORF Stop: TGA at 2119 |

| | | |
|---|---|---|
| SEQ ID NO: 114 | 642 aa | MW at 70935.5kD |

| | |
|---|---|
| NOV37a, | MGAARGSPARPRRLPLLSVLLLPLLGGTQTAIVFIKQPSSQDALQGRRALLRCEVEAP |
| CG110014-02 | |
| Protein Sequence | GPVHVYWLLDGAPVQDTERRFAQGSSLSFAAVDPLQDSGTFQCVARDDVTGEEARSAN |
| | ASFNIKWIEAGPVVLKHPASEAEIQPQTQVKLRCHIDGHPRPTYQWFRDGTPLSDGQS |
| | NHTVSSKKRNLTLRPAGPEHSGLYSCCAHSAFGQACSSQNFTLSIADESFARVVLAPQ |
| | DVVVARYEEAMFHCQFSAQPPPSLQWLFEDETPITNRSRPPHLRRATVFANGSLLLTQ |
| | VRPRNAGIYRCIGQGQRGPPIILEATLHLAEIEDMPLFEPRVFTAGSEERVTCLPPKG |
| | LPEPSVWWEHAGVRLPTHGRVYQKGHELVLANIAESDAGVYTCHAANLAGQRRQDANI |
| | TVATVPSWLKKPQDSQLEEGKPGYLDCLTQATPKPTVVWYRNQMLISEDSRFEVFKNG |
| | TLRINSVEVYDGTWYRCMSSTPAGSIEAQARVQVLEKLKFTPPPQPQQCMEFDKEATV |
| | PCSATGREKPTIKWERADGSSLPEWVTDNAGTLHFARVTRDDAGNYTCIASNGPQGQI |
| | RAHVQLTVAVFITFKVEPERTTVYPSKLYRLMQRCWALSPKDRPSFSEIASALGDSTV |
| | RAHVQLTVAVFITFKVEPERTTVYPSKLYRLMQRCWALSPKDRPSFSEIASALGDSTV |
| | DSKP |

| | |
|---|---|
| SEQ ID NO: 115 | 3693 bp |

| | |
|---|---|
| NOV37b, | CTTTTCCTGAGCCCGCCGCGATGGGAGCTGCGCGGGGATCCCCGGCCAGACCCCGCCG |
| CG110014-03 | |
| DNA Sequence | GTTGCCTCTGCTCAGCGTCCTGCTGCTGCCGCTGCTGGGCGGTACCCAGACAGCCATT |
| | GTCTTCATCAAGCAGCCGTCCTCCCAGGATGCACTGCAGGGGCGCCGGGCGCTGCTTC |
| | GCTGTGAGGTTGAGGCTCCGGGCCCGGTACATGTGTACTGGCTGCTCGATGGGGCCCC |
| | TGTCCAGGACACGGAGCGGCGTTTCGCCCAGGGCAGCAGCCTGAGCTTTGCAGCTGTG |
| | GACCGGCTGCAGGACTCTGGCACCTTCCAGTGTGTGGCTCGGGATGATGTCACTGGAG |
| | AAGAAGCCCGCAGTGCCAACGCCTCCTTCAACATCAAATGGATTGAGGCAGGTCCTGT |
| | GGTCCTGAAGCATCCAGCCTCGGAAGCTGAGATCCAGCCACAGACCCAGGTCACACTT |
| | CGTTGCCACATTGATGGGCACCCTCGATGAAAGCTTTGCCAGGGTGGTGCTGGCACCC |
| | CAGGACGTGGTAGTAGCGAGGTATGAGGAGGCCATGTTCCATTGCCAGTTCTCAGCCC |
| | AGCCACCCCCGAGCCTGCAGTGGCTCTTTGAGGATGAGACTCCCATCACTAACCGCAG |
| | TCGCCCCCCACACCTCCGCAGAGCCACAGTGTTTGCCAACGGGTCTCTGCTGCTGACC |
| | CAGGTCCGGCCACGCAATGCAGGGATCTACCGCTGCATTGGCCAGGGGCAGAGGGGCC |
| | CACCCATCATCCTGGAAGCCACACTTCACCTAGCAGAGATTGAAGACATGCCGCTATT |
| | TGAGCCACGGGTGTTTACAGCTGGCAGCGAGGAGCGTGTGACCTGCCTTCCCCCCAAG |
| | GGTCTGCCAGAGCCCAGCGTGTGGTGGGAGCACGCGGGAGTCCGGCTGCCCACCCATG |
| | GCAGGGTCTACCAGAAGGGCCACGAGCTGGTGTTGGCCATJATTGCTGAAAGTGATGC |
| | TGGTGTCTACACCTGCCACGCGGCCAACCTGGCTGGTCAGCGGAGACAGGATGTCAAC |
| | ATCACTGTGGCCACTGTGCCCTCCTGGCTGAAGAAGCCCCAAGACAGCCAGCTGGAGG |
| | AGGGCAAACCCGGCTACTTGGATTGCCTGACCCAGGCCACACCAAAACCTACAGTTGT |
| | CTGGTACAGAAACCAGATGCTCATCTCAGAGGACTCACGGTTCGAGGTCTTCAAGAAT |
| | GGGACCTTGCGCATCAACAGCGTGGAGGTGTATGATGGGACATGGTACCGTTGTATGA |
| | GCAGCACCCCAGCCGGCAGCATCGAGGCGCAAGCCCGTGTCCAAGTGCTGGAAAAGCT |
| | CAAGTTCACACCACCACCCCAGCCACAGCAGTGCATGGAGTTTGACAAGGAGGCCACG |
| | GTGCCCTGTTCAGCCACAGGCCGAGAGAAGCCCACTATTAAGTGGGAACGGGCAGATG |
| | GGAGCAGCCTCCCAGAGTGGGTGACAGACAACGCTGGGACCCTGCATTTTGCCCGGGT |
| | GACTCGAGATGACGCTGGCAACTACACTTGCATTGCCTCCAACGGGCCGCAGGGCCAG |
| | ATTCGTGCCCATGTCCAGCTCACTGTGGCAGTTTTTATCACCTTCAAAGTGGAACCAG |
| | AGCGTACGACTGTGTACCAGGGCCACACAGCCCTACTGCAGTGCGAGGCCCAGGGAGA |
| | CCCCAAGCCGCTGATTCAGTGGAAAGGCAAGGACCGCATCCTGGACCCCACCAAGCTG |
| | GGACCCAGGATGCACATCTTCCAGAATGGCTCCCTGGTGATCCATGACGTGGCCCCTG |
| | AGGACTCAGGCCGCTACACCTGCATTGCAGGCAACAGCTGCAACATCAAGCACACGGA |
| | GGCCCCCCTCTATGTCGTGGACAAGCCTGTGCCGGAGGAGTCGGAGCGCCCTGGCAGC |
| | CCTCCCCCCTACAAGATGATCCAGACCATTGGGTTGTCGGTGGGTGCCGCTGTGGCCT |
| | ACATCATTGCCGTGCTGGGCCTCATGTTCTACTGCAAGAAGCGCTGCAAAGCCAAGCG |
| | GCTGCAGAAGCAGCCCGAGGGCGAGGAGCCAGAGATGGAATGCCTCAACGGTGGGCCT |
| | TTGCAGAACGGGCAGCCCTCAGCAGAGATCCAAGAAGAAGTGGCCTTGACCAGCTTGG |
| | GCTCCGGCCCCGCGGCCCCCAACAAACGCCACAGCACAAGTGATAAGATGCACTTCCC |
| | ACGGTCTAGCCTGCAGCCCATCACCACGCTGGGGAAGAGTGAGTTTGGGGAGGTGTTC |
| | CTGGCAAAGGCTCAGGGCTTGGAGGAGGGAGTGGCAGAGACCCTGGTACTTGTGAAGA |
| | GCCTGCAGAGCAAGGATGAGCAGCAGCAGCTGGACTTCCGGAGGGAGTTGGAGATGTT |
| | TGGGAAGCTGAACCACGCCAACGTGGTGCGGCTCCTGGGGCTGTGCCGGGAGGCTGAG |
| | CCCCACTACATGGTGCTGGAATATGTGGATCTGGGAGACCTCAAGCAGTTCCTGAGGA |
| | TTTCCAAGAGCAAGGATGAAAAATTGAAGTCACGGCCCCTCAGCACCAAGCAGAAGGT |
| | GGCCCTATGCACCCAGGTAGCCCTGGGCATGGAGCACCTGTCCAACAACCGCTTTGTG |
| | CATAAGGACTTGGCTGCGCGTAACTGCCTGGTCAGTGCCCAGAGACAAGTGAAGGTGT |
| | CTGCCCTGGGCCTCAGCAAGGATGTGTACAACAGTGAGTACTACCACTTCCGCCAGGC |
| | CTGGGTGCCGCTGCGCTGGATGTCCCCCGAGGCCATCCTGGAGGGTGACTTCTCTACC |
| | AAGTCTGATGTCTGGGCCTTCGGTGTGCTGATGTGGGAAGTGTTTACACATGGAGAGA |
| | TCCCCCATGGTGGGCAGGCAGATGATGAAGTACTGGCAGATTTGCAGGCTGGGAAGGC |
| | TAGACTTCCTCAGCCCGAGGGCTGCCCTTCCAAACTCTATCGGCTGATGCAGCGCTGC |
| | TGGGCCCTCAGCCCCAAGGACCGGCCCTCCTTCAGTGAGATTGCCAGCGCCCTGGGAG |
| | ACAGCACCGTGGACAGCAAGCCGTGAGGAGGGAGCCCGCTCAGGATGGCCTGGGCAGG |
| | GGAGGACATCTCTAGAGGGAAGCTCACAGCATGATGGGCAAGATCCCTGTCCTCCTGG |
| | GCCCTGAGGCCCCTGCCCTAGTGCAACAGGCATTGCTGAGGTCTGAGCAGGGCCTGGC |
| | CTTTCCTCCTCTTCCTCACCCTCATCCTTTGGGAGGCTGACTTGGACCCAAACTGGGC |
| | GACTAGGGCTTTGAGCTGGGCAGTTTTCCCTGCCACCTCTTCCTCTATCAGGGACAGT |
| | GTGGGTGCCACAGGTAACCCCAATTTCTGGCCTTCAACTTCTCCCCTTGACCGGGTCC |
| | AACTCTGCCACTCATCTGCCAACTTTGCCTGGGGAGGGCTAGGCTTGGGATGAGCTGG |
| | GTTTGTGGGGAGTTCCTTAATATTCTCAAGTTCTGGGCACACAGGGTTAATGAGTCTC |
| | TTGGCCCACTGGTCCCACTTGGGGGTCTAGACCAGGATTATAGAGGACACAGCAAGTG |
| | AGTCCTCCCCACTCTGGGCTTGTGCACACTGACCCAGACCCACGTCTTCCCCACCCTT |
| | CTCTCCTTTCCTCATCCTAAGTGCCTGGCAGATGAAGGAGTTTTCAGGAGCTTTTGAC |
| | ACTATATAAACCGCCCTTTTCGTATGCACCACGGGCGGC |

| | |
|---|---|
| ORF Start: ATG at 478 | ORF Stop: TGA at 3040 |

| | | |
|---|---|---|
| SEQ ID NO: 116 | 854 aa | MW at 95008.5kD |

| | |
|---|---|
| NOV37b, | MGTLDESFARVVLAPQDVVVARYEEANFHCQFSAQPPPSLQWLFEDETPITNRSRPPH |
| CG110014-03 | |
| Protein Sequence | LRRATVFANGSLLLTQVRPRNAGIYRCIGQGQRGPPIILEATLHLAEIEDMPLFEPRV |
| | FTAGSEERVTCLPPKGLPEPSVWWEHAGVRLPTHGRVYQKGHELVLANIAESDAGVYT |
| | CHAANLAGQRRQDVNITVATVPSWLKKPQDSQLEEGKPGYLDCLTQATPKPTVVWYRN |
| | QMLISEDSRFEVFKNGTLRINSVEVYDGTWYRCMSSTPAGSIEAQARVQVLEKLKFTP |
| | PPQPQQCMEFDKEAIVPCSATGREKPTIKWERADGSSLPEWVTDNAGTLHFARVTRDD |
| | AGNYTCIASNGPQGQIRAHVQLTVAVFITFKVEPERTTVYQGHTALLQCEAQGDPKPL |
| | IQWKGKDRILDPTKLGPRMHIFQNGSLVIHDVAPEDSGRYTCIAGNSCNIKHTEAPLY |
| | VVDKPVPEESEGPGSPPPYKMIQTIGLSVGAATAYIIAVLGLMFYCKKRCKAKRLQKQ |
| | PEGEEPEMECLNGGPLQNGQPSAEIQEEVALTSLGSGPAAPNKRHSTSDKMHFPRSSL |
| | QPITTLGKSEFGEVFLAKAQGLEEGVAETLVLVKSLQSKDEQQQLDFRRELEMFGKLN |
| | HANVVRLLGLCREAEPHYMVLEYVDLGDLKQFLRISKSKDEKLKSRPLSTKQKVALCT |
| | QVALGMEHLSNNRFVHKDLAARNCLVSAQRQVKVSALGLSKDVYNSEYYHFRQAWVPL |
| | RWMSPEAILEGDFSTKSDVWAFGVLMWEVFTHGEMPHGGQADDEVLADLQAGKARLPQ |
| | PEGCPSKLYRLMQRCWALSPKDRPSFSEIASALGDSTVDSKP |

| | |
|---|---|
| SEQ ID NO: 117 | 2866 bp |

| | |
|---|---|
| NOV37c, | CTCAGCTCCTTTTCCTGAGCCCGCCGCGATGGGAGCTGCGCGGGGATCCCCGGCCAGA |
| CG110014-04 | |
| DNA Sequence | CCCCGCCGGTTGCCTCTGCTCAGCGTCCTGCTGCTGCCGCTGCTGGGCGGTACCCAGA |
| | CAGCCATTGTCTTCATCAAGCAGCCGTCCTCCCAGGATGCACTGCAGGGGCGCCGGGC |
| | GCTGCTTCGCTGTGAGGTTGAGGCTCCGGGCCCGGTACATGTGTACTGGCTGCTCGAT |
| | GGGGCCCCTGTCCAGGACACGGAGCGGCGTTTCGCCCAGGGCAGCAGCCTGAGCTTTG |
| | CAGCTGTGGACCGGCTGCAGGACTCTGGCACCTTCCAGTGTGTGGCTCGGGATGATGT |
| | CACTGGAGAAGAAGCCCGCAGTGCCAACGCCTCCTTCAACATCAAATGGATTGAGGCA |
| | GGTCCTGTGGTCCTGAAGCATCCAGCCTCGGAAGCTGAGATCCAGCCACAGACCCAGG |
| | TCACACTTCGTTGCCACATTGATGGGCACCCTCGGCCCACCTACCAATGGTTCCGAGA |
| | TGGGACCCCCCTTTCTGATGGTCAGAGCAACCACACAGTCAGCAGCAAGGAGCGGAAC |
| | CTGACGCTCCGGCCAGCTGGTCCTGAGCATAGTGGGCTGTATTCCTGCTGCGCCCACA |
| | GTGCTTTTGGCCAGGCTTGCAGCAGCCAGAACTTCACCTTGAGCATTGCTGATGAAAG |
| | CTTTGCCAGGGTGGTGCTGGCACCCCAGGACGTGGTAGTAGCGAGGTATGAGGAGGCC |
| | ATGTTCCATTGCCAGTTCTCAGCCCAGCCACCCCCGAGCCTGCAGTGGCTCTTTGAGG |
| | ATGAGACTCCCATCACTAACCGCAGTCGCCCCCCACACCTCCGCAGAGCCACAGTGTT |
| | TGCCAACGGGTCTCTGCTGCTGACCCAGGTCCGGCCACGCAATGCAGGGATCTACCGC |
| | TGCATTGGCCAGGGGCAGAGGGGCCCACCCATCATCCTGGAAGCCACACTTCACCTAG |
| | CAGAGATTGAAGACATGCCGCTATTTGAGCCACGGGTGTTTACAGCTGGCAGCGAGGA |
| | GCGTGTGACCTGCCTTCCCCCCAAGGGTCTGCCAGAGCCCAGCGTGTGGTGGGAGCAC |
| | GCGGGAGTCCGGCTGCCCACCCATGGCAGGGTCTACCAGAAGGGCCACGAGCTGGTGT |
| | TGGCCAATATTGCTGAAAGTGATGCTGGTGTCTACACCTGCCACGCGGCCAACCTGGC |
| | TGGTCAGCGGAGACAGGATGTCAACATCACTGTGGCCACTGTGCCCTCCTGGCTGAAG |
| | AAGCCCCAAGACAGCCAGCTGGAGGAGGGCAAACCCGGCTACTTGGATTGCCTGACCC |
| | AGGCCACACCAAAACCTACAGTTGTCTGGTACAGAAACCAGATGCTCATCTCAGAGGA |
| | CTCACGGTTCGAGGTGTTCCTGGCAAAGGCTCAGGGCTTGGAGGAGGGAGTGGCAGAG |
| | ACCCTGGTACTTGTGAAGAGCCTGCAGAGCAAGGATGAGCAGCAGCAGCTGGACTTCC |
| | GGAGGGAGTTGGAGATGTTTGGGAAGCTGAACCACGCCAACGTGGTGCGGCTCCTGGG |
| | GCTGTGCCGGGAGGCTGAGCCCCACTACATGGTGCTGGAATATGTGGATCTGGGAGAC |
| | CTCAAGCAGTTCCTGAGGATTTCCAAGAGCAAGGATGAAAAATTGAAGTCACAGCCCC |
| | TCAGCACCAAGCAGAAGGTGGCCCTATGCACCCAGGTAGCCCTGGGCATGGAGCACCT |
| | GTCCAACAACCGCTTTGTGCATAAGGACTTGGCTGCGCGTAACTGCCTGGTCAGTGCC |
| | CAGAGACAAGTGAAGGTGTCTGCCCTGGGCCTCAGCAAGGATGTGTACAACAGTGAGT |
| | ACTACCACTTCCGCCAGGCCTGGGTGCCGCTGCGCTGGATGTCCCCCGAGGCCATCCT |
| | GGAGGGTGACTTCTCTACCAAGTCTGATGTCTGGGCCTTCGGTGTGCTGATGTGGGAA |
| | GTGTTTACACATGGAGAGATGCCCCATGGTGGGCAGGCAGATGATGAAGTACTGGCAG |
| | ATTTGCAGGCTGGGAAGGCTAGACTTCCTCAGCCCGAGGGCTGCCCTTCCAAACTCTA |
| | TCGGCTGATGCAGCGCTGCTGGGCCCTCAGCCCCAAGGACCGGCCCTCCTTCAGTGAG |
| | ATTGCCAGCGCCCTGGGAGACAGCACCGTGGACAGCAAGCCGTGAGGAGGGAGCCCGC |
| | TCAGGATGGCCTGGGCAGGGGAGGACATCTCTAGAGGGAAGCTCACAGCATGATGGGC |
| | AAGATCCCTGTCCTCCTGGGCCCTGAGGCCCCTGCCCTAGTGCAACAGGCATTGCTGA |
| | GGTCTGAGCAGGGCCTGGCCTTTCCTCCTCTTCCTCACCCTCATCCTTTGGGAGGCTG |
| | ACTTGGACCCAAACTGGGCGACTAGGGCTTTGAGCTGGGCAGTTTTCCCTGCCACCTC |
| | TTCCTCTATCAGGGACAGTGTGGGTGCCACAGGTAACCCCAATTTCTGGCCTTCAACT |
| | TCTCCCCTTGACCGGGTCCAACTCTGCCACTCATCTGCCAACTTTGCCTGGGGAGGGC |
| | TAGGCTTGGGATGAGCTGGGTTTGTGGGGAGTTCCTTAATATTCTCAAGTTCTGGGCA |
| | CACAGGGTTAATGAGTCTCTTGGCCCACTGGTCCCACTTGGGGGTCTAGACCAGGATT |
| | ATAGAGGACACAGCAAGTGAGTCCTCCCCACTCTGCGCTTGTGCACACTGACCCAGAC |
| | CCACGTCTTCCCCACCCTTCTCTCCTTTCCTCATCCTAAGTGCCTGGCAGATGAAGGA |
| | GTTTTCAGGAGCTTTTGACACTATATAAACCGCCCTTTTTGTATGCACCACGGGCGGC |
| | TTTTATATGTAATTGCAGCGTGGG |

| | |
|---|---|
| ORF Start: ATG at 29 | ORF Stop: TGA at 2189 |

| | | |
|---|---|---|
| SEQ ID NO: 118 | 720 aa | MW at 80086.2kD |

| | |
|---|---|
| NOV37c, | MGAARGSPARPRRLPLLSVLLLPLLGGTQTAIVFIKQPSSQDALQGRRALLRCEVEAP |
| CG110014-04 | |
| Protein Sequence | GPVHVYWLLDGAPVQDTERRFAQGSSLSFAAVDRLQDSGTFQCVARDDVTGEEARSAN |
| | ASFNIKWIEAGPVVLKHPASEAEIQPQTQVThRCHIDGHPRPTYQWFRDGTPLSDGQS |
| | NHTVSSKERNLTLRPAGPEHSGLYSCCAHSAFGQACSSQNFTLSIADESFARVVLAPQ |
| | DVVVARYEEAMFHCQFSAQPPPSLQWLFEDETPITNRSRPPHLRRATVFANGSLLLTQ |
| | VRPRNAGIYRCIGQGQRGPPIILEATLHLAEIEDMPLFEPRVFTAGSEERVTCLPPKG |
| | LPEPSVWWEHAGVRLPTHGRVYQKGHELVLANIAESDAGVYTCHAANLAGQRRQDVNI |
| | TVATVPSWLKKPQDSQLEEGKPGYLDCLTQATPKPTVVWYRNQMLISEDSRFEVFLAK |
| | AQGLEEGVAETLVLVKSLQSKDEQQQLDFRRELEMFGKLNHANVVRLLGLCREAEPHY |
| | MVLEYVDLGDLKQFLRISKSKDEKLKSQPLSTKQKVALCTQVALGMEHLSNNRFVHKD |
| | LAARNCLVSAQRQVKVSALGLSKDVYNSEYYHFRQAWVPLRWMSPEAILEGDFSTKSD |
| | VWAFGVLMWEVFTHGEMPHGGQADDEVLADLQAGKARLPQPEGCPSKLYRLMQRCWAL |
| | SPKDRPSFSEIASALGDSTVDSKP |

Sequence comparison of the above protein sequences yields the following sequence relationships shown in Table 37B.

TABLE 37B
Comparison of NOV37a against NOV37b and NOV37c.

| | NOV37a Residues/ | Identities/Similarities |
|---|---|---|
| Protein Sequence | Match Residues | for the Matched Region |
| NOV37b | 221 . . . 604 | 383/384 (99%) |
| | 5 . . . 388 | 383/384 (99%) |
| NOV37c | 28 . . . 461 | 431/434 (99%) |
| | 28 . . . 461 | 431/434 (99%) |

Further analysis of the NOV37a protein yielded the following properties shown in Table 37C.

TABLE 37C
Protein Sequence Properties NOV37a

| | |
|---|---|
| PSort | 0.6950 probability located in outside; 0.1900 probability |
| analysis: | located in lysosome (lumen); 0.1900 probability |
| | located in plasma membrane; 0.1363 probability |
| | located in microbody (peroxisome) |
| SignalP | Cleavage site between residues 31 and 32 |
| analysis: | |

A search of the NOV37a protein against the Geneseq database, a proprietary database that contains sequences published in patents and patent publication, yielded several homologous proteins shown in Table 37D.

TABLE 37D
Geneseq Results for NOV37a

| | | NOV37a | Identities/ | |
|---|---|---|---|---|
| | | Residues/ | Similarities for | |
| Geneseq | Protein/Organism/Length [Patent | Match | the Matched | Expect |
| Identifier | #, Date] | Residues | Region | Value |
| AAW08747 | Human colon carcinoma kinase 4 | 1 . . . 604 | 599/604 (99%) | 0.0 |
| | (CCK-4) - Homo sapiens, 1070 aa. | 1 . . . 604 | 600/604 (99%) | |
| | [WO9637610-A2, 28-NOV-1996] | | | |
| ABB68257 | Drosophila melanogaster | 128 . . . 588 | 136/486 (27%) | 5e−40 |
| | polypeptide SEQ ID NO: 31563 - | 56 . . . 531 | 217/486 (43%) | |
| | Drosophila melanogaster, 1395 aa. | | | |
| | [WO200171042-A2, 27-SEP-2001] | | | |
| AAY08404 | Human ROBO1 protein - Homo | 128 . . . 602 | 144/508 (28%) | 1e−39 |
| | sapiens, 1649 aa. [WO9920764-A1, | 68 . . . 555 | 216/508 (42%) | |
| | 29-APR-1999] | | | |
| AAY13566 | Human Robo 1 polypeptde - Homo | 128 . . . 602 | 144/508 (28%) | 1e−39 |
| | sapiens, 1651 aa. [WO9925833-A1, | 68 . . . 555 | 216/508 (42%) | |
| | 27-MAY-1999] | | | |
| AAY08401 | Drosophila sp. ROBO1 protein - | 128 . . . 588 | 135/486 (27%) | 2e−39 |
| | Drosophila sp, 1395 aa. | 56 . . . 531 | 217/486 (43%) | |
| | [WO9920764-A1, 29-APR-1999] | | | |

In a BLAST search of public sequence databases, the NOV37a protein was found to have homology to the proteins shown in the BLASTP data in Table 37E.

TABLE 37E
Public BLASTP Results for NOV37a

| | | NOV37a | Identities/ | |
|---|---|---|---|---|
| Protein | | Residues/ | Similarities for | |
| Accession | | Match | the Matched | Expect |
| Number | Protein/Organism/Length | Residues | Portion | Value |
| JC4593 | protein-tyrosine kinase-related | 1 . . . 604 | 601/604 (99%) | 0.0 |
| | receptor PTK7 precursor - human, | 1 . . . 604 | 601/604 (99%) | |
| | 1070 aa. | | | |
| AAC50484 | TRANSMEMBRANE | 1 . . . 604 | 601/604 (99%) | 0.0 |
| | RECEPTOR PRECURSOR - | 1 . . . 604 | 601/604 (99%) | |
| | Homo sapiens (Human), 1070 aa. | | | |
| Q13308 | Tyrosine-protein kinase-like 7 | 1 . . . 604 | 599/604 (99%) | 0.0 |
| | precursor (Colon carcinoma kinase- | 1 . . . 604 | 600/604 (99%) | |
| | 4) (CCK-4) - Homo sapiens | | | |
| | (Human), 1070 aa. | | | |
| Q91048 | Tyrosine-protein kinase-like 7 | 17 . . . 604 | 382/589 (64%) | 0.0 |
| | precursor (Kinase like protein) - | 4 . . . 585 | 462/589 (77%) | |
| | Gallus gallus (Chicken), 1051 aa. | | | |
| Q9NSQ6 | HYPOTHETICAL 40.9 KDA | 357 . . . 461 | 104/105 (99%) | 5e−56 |
| | PROTEIN - Homo sapiens | 1 . . . 105 | 104/105 (99%) | |
| | (Human), 364 aa (fragment). | | | |

PFam analysis predicts that the NOV37a protein contains the domains shown in the Table 37F.

TABLE 37F
Domain Analysis of NOV37a

| | | Identities/ | |
|---|---|---|---|
| | NOV37a | Similarities | Expect |
| Pfam Domain | Match Region | for the Matched Region | Value |
| ig | 46 . . . 103 | 17/60 (28%) | 9.7e−07 |
| | | 42/60 (70%) | |
| ig | 143 . . . 202 | 17/63 (27%) | 1.4e−09 |
| | | 48/63 (76%) | |
| ig | 239 . . . 303 | 14/68 (21%) | 0.00022 |
| | | 47/68 (69%) | |
| ig | 336 . . . 393 | 15/60 (25%) | 3e−05 |
| | | 38/60 (63%) | |
| ig | 426 . . . 483 | 16/61 (26%) | 0.019 |
| | | 37/61 (61%) | |
| ig | 517 . . . 572 | 15/59 (25%) | 3.1e−09 |
| | | 41/59 (69%) | |
| pkinase | 605 . . . 629 | 8/28 (29%) | 0.024 |
| | | 20/28 (71%) | |

### Example 38

The NOV38 clone was analyzed, and the nucleotide and encoded polypeptide sequences are shown in Table 38A.

TABLE 38A
NOV38 Sequence Analysis

| | |
|---|---|
| SEQ ID NO: 119 | 2894 bp |

| | |
|---|---|
| NOV38a, | GATGGTGGGCTGTGGGGTGGCAGTTTTATGTTTGTGGGTTTCCTGCGGCGCTGCAGCG |
| CG110187-01 | |
| DNA Sequence | GGACAGCTCGAGTACTCAGTGCCGGAGGAGACGGAGCGGGGCGTAGCCGTAGGCAATC |
| | TCTCCGCGGACTTGAGGCTGCCAGCGGCCGCTATGTCCTCGCGGAACTTTCGCTTCCT |
| | TTCCAGCCACCGCGAGCTCTACTTCGGGGTGGATCTACCCAGCGGCAATTTGGTGGTC |
| | AGAGAGCCGGCGGACCGCGAACAGCTGTGCAGGGCCAAAGCTGCCTGCGTCTTGACCT |
| | ACGACCTGGTGCTCGAGGACCCGCTGGAGCTGCACAAGATTCGGATTCACGTCCTGGA |
| | CACCAATGACAACTCACCTCTCTTTCCTGCCGGCGACGTGCAGCTGCACATCCCCGAG |
| | TTCCTGACGCCCGGAGCCCGCTTTACTCTCCCGAATGCCCAAGATGACGACGAGGGAA |
| | GCAATGGGATACTAAGCTACAGCCTAAGCCCCAGTCAGCACTTTCGCCTGGACATGGG |
| | ATCGCGGGTTGACGGCAGCGAATACCCGGAGTTGGTGTTGGAGAAAGCACTGGATCGC |
| | GAACAGCGCGCCACCCACCTGCTGGTGCTTACAGCTCGGGACGGCGGGCTACCTGCCC |
| | GCTCAGGAGACGCACAAGTCACCATCATTGTGGTGGACACAAATGACAACGCGCCTGT |
| | ATTTGAGCGCTCCGTATACCGCACCAAGGTTCCAGAGACTGCACCCAATGGGACTGTG |
| | TTATTCCGAGTTCAAGCCTTGGATCCAGATGAAGGGTCCAATGGGGAAGTCCAATACT |
| | CCCTAAGCAACAGCACGCAAGCAGAGCTGCGACACCGCTTTCACGTTCACCCTAAAAG |
| | TGGGGAGGTGCAAGTAGCTGCTTCACTAGGTCCGCCTGAAACGCTCTTGGAGGCATAC |
| | ATTGAGGCGAGGGACGAAGGTGTCTTTGGTTTAGCTAGCACCGCTAAACTGCTGGTGG |
| | AGGTGACTGACGTGAACGATCATGCCCCCGAACTGGACTTCCTGACTCTTTCGAACCC |
| | AGTACCTGAGGACGCTGCCCCTGGCACAGTGATTGCTCTCTTTAGTGTAAAGGATGAA |
| | GACCTCGATTCTAATGGTAGGGTCATTTGTGGCATGTCTAGTGCAGGCCCTTTTCAGC |
| | TGACGGCTTCCTTTGACAACTACTACAGCCTGCTGATTGATGGGCCCCTGGACCGGGA |
| | GCAGATCAGTGAATACCAAGTCCTGATCACGGCCTCAGATAGTGGCTCACCCCCACTT |
| | AGCACCCGAAGGACAATCACTGTGTCAGTTGCTGATGTGAATGACAATACACCAAACT |
| | TTCCTCAACCCCAGCAGGAACTTTTCGTTGCTGAAAACAATGGCCCTGGGGCCTCTCT |
| | AGGCCGAGTGTTTGCCCAGGACCCCGACCTGGGGAAGAATGGCCTTGTCTCTTATGAG |
| | CTGTTGGATGTTATCTCTGAACGGCCATCAGCCTCTAGCTTGGTGGCAGTGGAATCAG |
| | CCAGTGGGGCCATCACTGCCAAAACTTCCTTTGACTTTGAGCAGCTCAGGGGGTTTCA |
| | TTTCCAAGTAGAGGGCCGGGATGGTGGCATTCCTCCCAGAAGTGCAACAGTGACTATA |
| | AACTTGTTTGTGGTAGATAGGAATGACAATTATCCGGTTATCTTCGTTCCCTTGCCCA |
| | GAAATGGTTCTGTCCCAGTGGAAATTGTGCCCCGCTCTGCCAGGACTGGACACTTGGT |
| | CACAAAAGTGGTAGCAGAGGATGCTGACAGTGGTTCTAATGCCTGGCTTTCCTACCAC |
| | ATCTCCCGGGCGTCTGACTCTAGTCTCTTTAGAATTTCAGCCAATATAGGTGAGCTCC |
| | GTACTGCTCGCTTAGTTCTTCCCACTGATGCAGTTAAGCAGAGGGTGGTGGTAGTGGT |
| | TCGGGACCATGGAGACCCACCACTTTCCTCCTCTGTCACTCTGGGTGTGCTGTTGAGC |
| | AACTCTGTCCCTCAGTTACTTCCAGACTTTGAAGATGTCTGGGAACCAGGAGGGCAGC |
| | TTTCTGCCCAGAACTTGTATTTAGTAATTGCCTTGGCTTGTATTTCCTTTTTATTTCT |
| | GGGGTGCTTACTTTTCTTCGTGTGTACCAAGTTGCACCAGAGCCCAGGCTGTTGCGCT |
| | CAGAGCTGCTGTCGCTCTACAGAGGATCTGAGGTATGGAAGTAAGATGGTTTCAAATC |
| | CTTGCATGACATCAGCCACCATAGATGTCACTACAGTTGAGAGACTTTCTCAGACTTA |
| | TCTCTATCGGGCCTCTCTGGGACTTGGTTCTGATAATAACAGTTTGCTGTTGCGTGGG |
| | GAGTACAATGCTGCCGACCTGCGAAATCTTGCCACTGGGGTAGGACTGAATTTGCCAA |
| | TATCCTGTATTCAGATTCGGAATAGGAAAGGGGATCACGCTAATGTCAATGCCATGCC |
| | ACGACAGCCCAACCCTGACTGGCGTTACTCTGCCTCCCTGAGAGCAGGCATGCACAGC |
| | TCTGTGCACCTAGAGGAGGCTGGCATTCTACGGGCTGGTCCAGGAGGGCCTGATCAGC |
| | AGTGGCCAACAGTATCCAGTGCAACACCAGAACCAGAGGCAGGAGAAGTGTCCCCTCC |
| | AGTCGGTGCGGGTGTCAACAGCAACAGCTGGACCTTTAAATACGGACCAGGCAACCCC |
| | AAACAATCCGGTCCCGGTGAGTTGCCCGACAAATTCATTATCCCAGGATCTCCTGCAA |
| | TCATCTCCATCCGGCAGGAGCCTACTAACAGCCAAATTGACAAAAGTGACTTCATAAC |
| | CTTCGGCAAAAAGGAGGAGACCAAGAAAAAGAAGAAAAAGAAGAAGGGTAACAAGACC |
| | CAGGAGAAAAAAGAGAAAGGGAACAGCACGACTGACAACGTGACCAGTGAG |

| | |
|---|---|
| ORF Start: ATG at 2 | ORF Stop: TGA at 2891 |

| | | |
|---|---|---|
| SEQ ID NO: 120 | 963 aa | MW at 103961.5kD |

| | |
|---|---|
| NOV38a, | MVGCGVAVLCLWVSCGAAAGQLEYSVPEETERGVAVGNLSADLRLPAAAMSSRNFRFL |
| CG110187-01 | |
| Protein Sequence | SSHRELYFGVDLPSGNLVVREPADREQLCPAKAACVLTYDLVLEDPLELHKIRIHVLD |
| | TNDNSPLFPAGDVQLHIPEFLTPGARFTLFNAQDDDEGSNGILSYSLSPSQHFRLDMG |
| | SRVDGSEYPELVLEKALDREQRATHLLVLTARDGGLPARSGDAQVTIIVVDTNDNAPV |
| | FERSVYRTKVPETAPNGTVLFRVQALDPDEGSNGEVQYSLSNSTQAELRHRFHVHPKS |
| | GEVQVAASLGPPETLLEAYIEARDEGVFGLASTAKLLVEVTDVNDHAPELDFLTLSNP |
| | VPEDAAPGTVIALFSVKDEDLDSNGRVICGMSSAGPFQLTASFDNYYSLLIDGPLDRE |
| | QISEYQVLITASDSGSPPLSTRRTITVSVADVNDNTPNFPQPQQELFVAENNGPGASL |
| | GRVFAQDPDLGKNGLVSYELLDVISERPSASSLVAVESASGAITAKTSFDFEQLRGFH |
| | FQVEGRDGGIPPRSATVTINLFVVDRNDNYPVILVPLPRNGSVPVEIVPRSARTGHLV |
| | TKVVAEDADSGSNAWLSYHISRASDSSLFRISANIGELRTARLVLPTDAVKQRVVVVV |
| | RDHGDPPLSSSVTLGVLLSNSVPQLLPDFEDVWEPGGQLSAQNLYLVIALACISFLFL |
| | GCLLFFVCTKLHQSPGCCAQSCCRSTEDLRYGSKMVSNPCMTSATIDVTTVERLSQTY |
| | LYRASLGLGSDNNSLLLRGEYNAADLRNLATGVGLNLPISCIQIRNRKGDHANVNAMP |
| | RQPNPDWRYSASLRAGMHSSVHLEEAGILRAGPGGFDQQWPTVSSATPEPEAGEVSFP |
| | VGAGVNSNSWTFKYGPGNPKQSGPGELPDKFIIPGSPAIISIRQEPTNSQIDKSDFIT |
| | FGKKEETKKKKKKKKGNKTQEKKEKGNSTTDNSDQ |

| | |
|---|---|
| SEQ ID NO: 121 | 2010 bp |

| | |
|---|---|
| Nov38b, | AGATCTGCGGGACAGCTCGAGTACTCAGTGCGGGAGGAGACGGAGCGGGGCGTAGCCG |
| CG110187-03 | |
| DNA Sequence | TAGGCAATCTCTCCGCGGACTTGAGGCTGCCAGCGGCCGCTATGTCCTCGCGGAACTT |
| | TCGCTTCCTTTCCAGCCACCGCGAGCTCTACTTCGGGGTGGATCTACCCAGCGGCAAT |
| | TTGGTGGTCAGAGAGCCGGCGGACCGCGAACAGCTGTGCAGGGCCAAAGCTGCCTGCG |
| | TCTTGACCTACGACCTGGTGCTCGAGGACCCGCTGGAGCTGCACAAGATTCGGATTCA |
| | CGTCCTGGACACCAATGACAACTCACCTCTCTTTCCTGCCGGCGACGTGCAGCTGCAC |
| | ATCCCCGAGTTCCTGACGCCCGGAGCCCGCTTTACTCTCCCGAATGCCCAZGATGACG |
| | ACGAGGGAAGCAATGGGATACTAAGCTACAGCCTAAGCCCCAGTCAGCACTTTCGCCT |
| | GGACATGGGATCGCGGGTTGACGGCAGCGAATACCCGGAGTTGGTGTTGGAGAAAGCA |
| | CTGGATCGCGAACAGCGCGCCACCCACCTGCTGGTGCTTACAGCTCGGGACGGCGGGC |
| | TACCTGCCCGCTCAGGAGACGCACAAGTCACCATCATTGTGGTGGACACAAATGACAA |
| | CGCGCCTGTATTTGAGCGCTCCGTATACCGCACCAAGGTTCCAGAGACTGCACCCAAT |
| | GGGACTGTGTTATTCCGAGTTCAAGCCTTGGATCCAGATGAAGGGTCCAATGGGGAAG |
| | TCCAATACTCCCTAAGCAACAGCACGCAAGCAGAGCTGCGACACCGCTTTCACGTGCA |
| | GAGGCATACATTGAGGCGAGGGACGAAGGTGTCTTTGGTTTAGCTAGCACCGCTAAAC |
| | TGCTGGTGGAGGTGACTGACGTGAACGATCATGCCCCCGAACTGGACTTCCTGACTCT |
| | TTCGAACCCAGTACCTGAGGACGCTGCCCCTGGCACAGTGATTGCTCTCTTTAGTGTA |
| | AAGGATGAAGACCTCGATTCTAATGGTAGGGTCATTTGTGGCATGTCTAGTGCAGGCC |
| | CTTTTCAGCTGACGGCTTCCTTTGACAACTACTACAGCCTGCTGATTGATGGGCCCCT |
| | GGACCGGGAGCAGATCAGTGAAAACCAAGTCCTGATCACGGCCTCAGATAGTGGCTCA |
| | CCGCCACTTAGCACCCGAAGGACAATCACTGTGTCAGTTGCTGATGTGAATGACAATA |
| | CACCAAACTTTCCTCAACCCCAGCAGGAACTTTTCGTTGCTGAAAACAATGGCCCTGG |
| | GGCCTCTCTAGGCCGAGTGTTTGCCCAGGACCCCGACCTGGGGAAGAATGGCCTTGTC |
| | TCTTATGAGCTGTTGGATGTTATCTCTGAAGGGCCATCAGCCTCTAGCTTGGTGGCAG |
| | TGGAATCATCCAGTGGGGCCATCACTGCCAAAACTTCCTTTGACTTTGAGCAGCTCAG |
| | GGGGTTTCATTTCCAAGTAGAAGGCCGGGATGGTGGCATTCCTCCCAGAAGTGCAACA |
| | GTGACTATAAACTTGTTTGTGGTAGATAGGAATGACAATTATCCGGTTATCTTGTTTC |
| | CCTTGCCCAGAAATGGTTCTGTCCCAGTGGAAATTGTGCCCCGCTCTGCCAGGACTGG |
| | ACACTTGGTCACAAAAGTGGTAGCAGAGGATGCTGACAGTGGTTCTAATGCCTGGCTT |
| | TCCTACCACATCTCCCGGGCGTCTGACTCTAGTCTCTTTAGAATTTCAGCCAATATAG |
| | GTGAGCTCCGTACTGCTCGCTTAGTTCTTCCCACTGATGCAGTTAAGCAGAGGGTGGT |
| | GGTAGTGGTTCGGGACCATGGAGACCCACCACTTTCCTCCTCTGTCACTCTGGGTGTG |
| | CTGTTGAGCAACTCTGTCCCTCAGTTACTTCCAGACTTTGAAGATGTCTGGGAACCAG |
| | GAGGGCAGCTTTCTGCCCAGAACTTGTATTTAGTCGAC |

| | |
|---|---|
| ORF Start: at 7 | ORF Stop: end of sequence |

| | | |
|---|---|---|
| SEQ ID NO: 122 | 688 aa | MW at 72305.7kD |

| | |
|---|---|
| NOV38b, | AGQLEYSVREETERGVAVGNLSADLRLPAAANSSRNFRFLSSHRELYFGVDLPSGNLV |
| CG110187-03 | |
| Protein Sequence | VREFADREQLCRAKAACVLTYDLVLEDPLELHKIRIHVLDTNDNSPLFPAGDVQLHIP |
| | EFLTPGARFTLPNAQDDDEGSNGILSYSLSPSQHFRLDMGSRVDGSEYPELVLEKALD |
| | REQRATHLLVLTARDGGLPARSGDAQVTIIVVDTNDNAPVFERSVYRTKVPETAPNGT |
| | VLFRVQALDPDEGSNGEVQYSLSNSTQAELRHRFHVHPKSGEVQVAASLGPFETLLEA |
| | YIEARDEGVFGLASTAKLLVEVTDVNDHAPELDFLTLSNPVPEDAAPGTVIALFSVKD |
| | EDLDSNGRVICGMSSAGPFQLTASFDNYYSLLIDGPLDREQISEYQVLITASDSGSPP |
| | LSTRRTITVSVADVNDNTPNFPQPQQELFVAENNGPGASLGRVFAQDPDLGKNGLVSY |
| | ELLDVISEGPSASSLVAVESSSGAITAKTSFDFEQLRGFHFQVEGRDGGIPPRSATVT |
| | INLFVVDRNDNYPVILFPLPRNGSVPVEIVPRSARTGHLVTKVVAEDADSGSNAWLSY |
| | HISRASDSSLFRISANIGELRTARLVLPTDAVKQRVVVVVRDHGDPPLSSSVTLGVLL |
| | SNSVPQLLPDFEDVWEPGGQLSAQNLYLVD |

Sequence comparison of the above protein sequences yields the following sequence relationships shown in Table 38B.

TABLE 38B
Comparison of NOV38a against NOV38b.

| Protein | NOV38a Residues/ | Identities/ |
|---|---|---|
| Sequence | Match Residues | Similarities for the Matched Region |
| NOV38b | 19 . . . 685 | 630/667 (94%) |
| | 1 . . . 667 | 630/667 (94%) |

Further analysis of the NOV38a protein yielded the following properties shown in Table 38C.

TABLE 38C
Protein Sequence Properties NOV38a

| | |
|---|---|
| PSort | 0.4600 probability located in plasma membrane; |
| analysis: | 0.2400 probability located in nucleus; 0.1000 probability |
| | located in endoplasmic reticulum (membrane); |
| | 0.1000 probability located in endoplasmic reticulum (lumen) |
| SignalP | Cleavage site between residues 19 and 20 |
| analysis: | |

A search of the NOV38a protein against the Geneseq database, a proprietary database that contains sequences published in patents and patent publication, yielded several homologous proteins shown in Table 38D.

TABLE 38D
Geneseq Results for NOV38a

| | | NOV38a | Identities/ | |
|---|---|---|---|---|
| | | Residues/ | Similarities for | |
| Geneseq | Protein/Organism/Length [Patent | Match | the Matched | Expect |
| Identifier | #, Date] | Residues | Region | Value |
| ABG15234 | Novel human diagnostic protein | 8 . . . 963 | 480/984 (48%) | 0.0 |
| | #15225 - Homo sapiens, 1008 aa. | 31 . . . 1008 | 637/984 (63%) | |
| | [WO200175067-A2, 11-OCT-2001] | | | |
| ABG15234 | Novel human diagnostic protein | 8 . . . 963 | 480/984 (48%) | 0.0 |
| | #15225 - Homo sapiens, 1008 aa. | 31 . . . 1008 | 637/984 (63%) | |
| | [WO200175067-A2, 11-OCT-2001] | | | |
| AAM78649 | Human protein SEQ ID NO: 1311 - | 3 . . . 941 | 368/957 (38%) | e−170 |
| | Homo sapiens, 931 aa. | 9 . . . 931 | 526/957 (54%) | |
| | [WO200157190-A2, 09-AUG-2001] | | | |
| AAM79633 | Human protein SEQ ID NO: 3279 - | 3 . . . 941 | 370/962 (38%) | e−169 |
| | Homo sapiens, 949 aa. | 21 . . . 949 | 528/962 (54%) | |
| | [WO200157190-A2, 09-AUG-2001] | | | |
| ABB12315 | Human protocadherin homologue, | 3 . . . 941 | 370/962 (38%) | e−169 |
| | SEQ ID NO: 2685 - Homo sapiens, | 21 . . . 949 | 528/962 (54%) | |
| | 949 aa. [WO200157188-A2, 09-AUG-2001] | | | |

In a BLAST search of public sequence databases, the NOV38a protein was found to have homology to the proteins shown in the BLASTP data in Table 38E.

TABLE 38E
Public BLASTP Results for NOV38a

| | | NOV38a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9H158 | Protocadherin alpha-C1 precursor | 1 . . . 963 | 956/963 (99%) | 0.0 |
| | (PCDH-alpha-C1) - Homo sapiens | 1 . . . 963 | 959/963 (99%) | |
| | (Human), 963 aa. | | | |
| Q91Y10 | PROTOCADHERIN ALPHA C1 - | 1 . . . 963 | 813/964 (84%) | 0.0 |
| | Mus musculus (Mouse), 964 aa. | 1 . . . 964 | 873/964 (90%) | |
| Q91Y09 | PROTOCADHERIN ALPHA C2 - | 16 . . . 963 | 484/980 (49%) | 0.0 |
| | Mus musculus (Mouse), 1006 aa. | 37 . . . 1006 | 636/980 (64%) | |
| Q9Y5I4 | Protocadherin alpha C2 precursor | 8 . . . 963 | 482/984 (48%) | 0.0 |
| | (PCDH-alpha-C2) - Homo sapiens | 30 . . . 1007 | 639/984 (63%) | |
| | (Human), 1007 aa. | | | |
| Q9Y5H6 | Protocadherin alpha 8 precursor | 8 . . . 963 | 455/963 (47%) | 0.0 |
| | (PCDH-alpha8) - Homo sapiens | 17 . . . 950 | 590/963 (61%) | |
| | (Human), 950 aa. | | | |

PFam analysis predicts that the NOV38a protein contains the domains shown in the Table 38F.

TABLE 38F
Domain Analysis of NOV38a

| | | Identities/ | |
|---|---|---|---|
| Pfam | NOV38a | Similarities | |
| Domain | Match Region | for the Matched Region | Expect Value |
| cadherin | 129 . . . 224 | 31/110 (28%) | 1.6e−10 |
| | | 65/110 (59%) | |
| cadherin | 238 . . . 331 | 31/110 (28%) | 6e−16 |
| | | 66/110 (60%) | |
| cadherin | 345 . . . 436 | 36/107 (34%) | 6.2e−10 |
| | | 64/107 (60%) | |
| cadherin | 450 . . . 546 | 28/112 (25%) | 5e−12 |
| | | 67/112 (60%) | |
| cadherin | 566 . . . 657 | 28/108 (26%) | 0.00086 |
| | | 60/108 (56%) | |

### Example 39

The NOV39 clone was analyzed, and the nucleotide and encoded polypeptide sequences are shown in Table 39A.

TABLE 39A
NOV39 Sequence Analysis

| | |
|---|---|
| SEQ ID NO: 123 | 3630 bp |

| | |
|---|---|
| NOV39a, | TGCGGCCGCGGAAAGAATGCGCGCCGCCCGTGCGCTCCGCCTGCCGCGTCTGGCCACC |
| CG110205-01 | |
| DNA Sequence | CGCAGCCGCCGCGTCCGCACCTGACCATGGAGTGCGCCCTCCTGCTCGCGTGTGCCTT |
| | CCCGGCTGCGGGTTCGGGCCCGCCGAGGGGCCTGGCGGGACTGGGGCGCGTGGCCAAG |
| | GCGCTCCAGCTGTGCTGCCTCTGCTGTGCGTCGGTCGCCGCGGCCTTAGCCAGTGACA |
| | GCAGCAGCGGCGCCAGCGGATTAAATGATGATTACGTCTTTGTCACGCCAGTAGAAGT |
| | AGACTCAGCCGGGTCATATATTTCACACGACATTTTGCACAACGGCAGGAAAAAGCGA |
| | TCGGCGCAGAATGCCAGAAGCTCCCTGCACTACCGATTTTCAGCATTTGGACAGGAAC |
| | TGCACTTAGAACTTAAGCCCTCGGCGATTTTGAGCAGTCACTTTATTGTCCAGGTACT |
| | TGGAAAAGATGGTGCTTCAGAGACTCAGAAACCCGAGGTGCAGCAATGCTTCTATCAG |
| | GGATTTATCAGAAATGACAGCTCCTCCTCTGTCGCTGTGTCTACGTGTGCTGGCTTGT |
| | CAGGTTTAATAAGGACACGAAAAAATGAATTCCTCATCTCGCCATTACCTCAGCTTCT |
| | GGCCCAGGAACACAACTACAGCTCCCCTGCGGGTCACCATCCTCACGTACTGTACAAA |
| | AGGACAGCAGAGGAGAAGATCCAGCGGTACCGTGGCTACCCCGGCTCTGGCCGGAATT |
| | ATCCTGGTTACTCCCCAAGTCACATTCCCCATGCATCTCAGAGTCGAGAGACAGAGTA |
| | TCACCATCGAAGGTTGCAAAAGCAGCATTTTTGTGGACGACGCAAGAAATATGCTCCC |
| | AAGCCTCCCACAGACGACACCTATCTAAGGTTTGATGAATATGGGAGCTCTGGGCGAC |
| | CCAGAAGATCAGCTGGAAAATCACAAAAGGGCCTCAATGTGGAAACCCTCGTGGTGGC |
| | AGACAAGAAAATGGTGGAAAAGCATGGCAAGGGAAATGTCACCACATACATTCTCACA |
| | GTAATGAACATGGTTTCTGGCCTATTTAAAGATGGGACTATTGGAAGTGACATAAACG |
| | TGGTTGTGGTGAGCCTAATTCTTCTGGAACAAGAACCTGGAGGATTATTGATCAACCA |
| | TCATGCAGACCAGTCTCTGAATAGTTTTTGTCAATGGCAGTCTGCCCTCATTGGAAAG |
| | AATGGCAAGAGACATGATCATGCCATCTTACTAACAGGATTTGATATTTGTTCTTGGA |
| | AGAATGAACCATGTGACACTCTAGGGTTTGCCCCCATCAGTGGAATGTGCTCTAAGTA |
| | CCGAAGTTGTACCATCAATGAGGACACAGGACTTGGCCTTGCCTTCACCATCGCTCAT |
| | GAGTCAGGGCACAACTTTGGTATGATTCACGATGGAGAAGGGAATCCCTGCAGAAAGG |
| | CTGAAGGCAATATCATGTCTCCCACACTGACCGGAAACAATGGAGTGTTTTCATGGTC |
| | TTCCTGCAGCCGCCAGTATCTCAAGAAATTCCTCAGCACACCTCAGGCGGGGTGTCTA |
| | GTGGATGAGCCCAAGCAAGCAGGACAGTATAAATATCCGGACAAACTACCAGGACAGA |
| | TTTATGATGCTGACACACAGTGTAAATGGCAATTTGGAGCAAAAGCCAAGTTATGCAG |
| | CCTTGGTTTTGTGAAGGATATTTGCAAATCACTTTGGTGCCACCGAGTAGGCCACAGG |
| | TGTGAGACCAAGTTTATGCCCGCAGCAGAAGGGACCGTTTGTGGCTTGAGTATGTGGT |
| | GTCGGCAAGGCCAGTGCGTAAAGTTTGGGGAGCTCGGGCCCCGGCCCATCCACGGCCA |
| | GTGGTCCGCCTGGTCGAAGTGGTCAGAATGTTCCCGGACATGTGGTGGAGGAGTCAAG |
| | TTCCAGGAGAGACACTGCAATAACCCCAAGCCTCAGTATGGTGGCTTATTCTGTCCAG |
| | GTTCTAGCCGTATTTATCAGCTGTGCAATATTAACCCTTGCAATGAAAATAGCTTGGA |
| | TTTTCGGGCTCAACAGTGTGCAGAATATAACAGCAAACCTTTCCGTGGATGGTTCTAC |
| | CAGTGGAAACCCTATACAAAAGTGGAAGAGGAAGATCGATGCAAACTGTACTGCAAGG |
| | CTGAGAACTTTGAATTTTTTTTTGCAATGTCCGGCAAACTGAAAGATGGAACTCCCTG |
| | CTCCCCAAACAAAAATGATGTTTGTATTGACGGGGTTTGTGAACTAGTGGGATGTGAT |
| | CATGAACTAGGCTCTAAAGCAGTTTCAGATGCTTGTGGCGTTTGCAAAGGTGATAATT |
| | CAACTTGCAAGTTTTATAAAGGCCTGTACCTCAACCAGCATAAAGCAAATGAATATTA |
| | TCCGGTGGTCCTCATTCCAGCTGGCGCCCGAAGCATCGAAATCCAGGAGCTGCAGGTT |
| | TCCTCCAGTTACCTCGCAGTTCGAAGCCTCAGTCAAAAGTATTACCTCACCGGGGGCT |
| | GGAGCATCCACTGGCCTGGGGAGTTCCCCTTCGCTGGGACCACGTTTGAATACCAGCG |
| | CTCTTTCAACCGCCCGGAACGTCTGTACGCGCCAGGGCCCACAAATGAGACGCTGATT |
| | CTGATGCAAGGCAAAAATCCAGGGATAGCTTGGAAGTATGCACTTCCCAAGGTCATGA |
| | ATGGAACTCCACCAGCCACAAAAAGACCTGCCTATACCTGCTGGATGCCAGGTGAATG |
| | GAGTACATGCAGCAAGGCCTGTGCTGGAGGCCAGCAGAGCCGAAAGATCCAGTGTGTG |
| | CAAAAGAAGCCCTTCCAAAAGGAGGAAGCAGTGTTGCATTCTCTCTGTCCAGTGAGCA |
| | CACCCACTCAGGTCCAAGCCTGCAACAGCCATGCCTGCCCTCCACAATGGAGCCTTGG |
| | ACCCTGGTCTCAGTGTTCCAAGACCTGTGGACGAGGGGTGAGGAAGCGTGAACTCCTC |
| | TGCAAGGGCTCTGCCGCAGAAACCCTCCCCGAGAGCCAGTGTACCAGTCTCCCCAGAC |
| | CTGAGCTGCAGGAGGGCTGTGTGCTTGGACGATGCCCCAAGAACAGCCGGCTACAGTG |
| | GGTCGCTTCTTCGTGGAGCGAGTGTTCTGCAACCTGTGGTTTGGGTGTGAGGAAGAGG |
| | GAGATGAAGTGCAGCGAGAAGGGCTTCCAGGGAAAGCTGATAACTTTCCCAGAGCGAA |
| | GATGCCGTAATATTAAGAAACCAAATCTGGACTTGGAAGAGACCTGCAACCGACGGGC |
| | TTGCCCAGCCCATCCAGTGTACAACATGGTAGCTGGATGGTATTCATTGCCGTGGCAG |
| | CAGTGCACAGTCACCTGTGGGGGAGGGGTCCAGACCCGGTCAGTCCACTGTGTTCAGC |
| | AAGGCCGGCCTTCCTCAAGTTGTCTGCTCCATCAGAAACCTCCGGTGCTACGAGCCTG |
| | TAATACAAACTTCTGTCCAGCTCCTGAAAAGAGAGAGGATCCATCCTGCGTAGATTTC |
| | TTCAACTGGTGTCACCTAGTTCCTCAGCATGGTGTCTGCAACCACAAGTTTTACGGAA |
| | AACAATGCTGCAAGTCATGCACAAGGAAGATCTGATCTTGGTGTCCTCCCCAGCACCT |
| | TATGGCCAGGGGCTTACCTTTCAACCTCTAGAGA |

| | |
|---|---|
| ORF Start: ATG at 85 | ORF Stop: TGA at 3571 |

| | | |
|---|---|---|
| SEQ ID NO: 124 | 1162 aa | MW at 128776kD |

| | |
|---|---|
| NOV39a, | MECALLLACAFPAAGSGPPRGLAGLGRVAKALQLCCLCCASVAAALASDSSSGASGLN |
| CG110205-01 | |
| Protein Sequence | DDYVFVTPVEVDSAGSYISIDILHNGRKKRSAQNARSSLHYRFSAFGQELHLELKPSA |
| | ILSSHFIVQVLGKDGASETQKPEVQQCFYQGFIRNDSSSSVAVSTCAGLSGLIRTRKN |
| | EFLISPLPQLLAQEHNYSSPAGHHPHVLYKRTAEEKIQRYRGYPGSGRNYPGYSPSHI |
| | PHASQSRETEYHHRRLQKQHFCGRRKKYAPKPPTEDTYLRFDEYGSSGRPRRSAGKSQ |
| | KGLNVETLVVADKKMVEKHGKGNVTTYILTVMNNVSGLFKDGTIGSDINVVVVSLILL |
| | EQEPGGLLINHHADQSLNSFCQWQSALIGKNGKRHDHAILLTGFDICSWKNEPCDTLG |
| | FAPISGMCSKYRSCTINEDTGLGLAFTIAHESGHNFGMIHDGBGNPCRKAEGNIMSPT |
| | LTGNNGVFSWSSCSRQYLKKFLSTPQAGCLVDEPKQAGQYKYPDKLPGQIYDADTQCK |
| | WQFGAKAKLCSLGFVKDICKSLWCHRVGHRCETKFMPAAEGTVCGLSMWCRQGQCVKF |
| | GELGPRPIHGQWSAWSKWSECSRTCGGGVKFQERHCNNPKPQYGGLFCPGSSRIYQLC |
| | NINPCNENSLDFRAQQCAEYNSKPFRGWFYQWKPYTKVEEEDRCKLYCKAENFEFFFA |
| | MSGKVKDGTPCSPNKNDVCIDGVCELVGCDHELGSKAVSDACGVCKGDNSTCKFYKGL |
| | YLNQHKANEYYPVVLIPAGARSIEIQELQVSSSYLAVRSLSQKYYLTGGWSIDWPGEF |
| | PFAGTTFEYQRSFNRPERLYAPGPTNETLILMQGKNPGIAWKYALPKVMNGTPPATKR |
| | PAYTCWMPGEWSTCSKACAGGQQSRKIQCVQKKPFQKEEAVLHSLCPVSTPTQVQACN |
| | SHACPFQWSLGPWSQCSKTCGRGVRKRELLCKGSAAETLPESQCTSLPRPELQEGCVL |
| | GRCPKNSRLQWVASSWSECSATCGLGVRKREMKCSEKGFQGKLITFPERRCRNIKKPN |
| | LDLEETCNRRACPAHPVYNMVAGWYSLPWQQCTVTCGGGVQTRSVHCVQQGRPSSSCL |
| | LHQKPPVLRACNTNFCPAPEKREDPSCVDFFNWCHLVPQHGVCNHKFYGKQCCKSCTR |
| | KI |

| | |
|---|---|
| SEQ ID NO: 125 | 1059 bp |

| | |
|---|---|
| NOV39b, | AAGCTTGTGGAAACCCTCGTGGTGGCAGACAAGCAAATGGTGGAAAAGCATGGCAAGG |
| CG110205-02 | |
| DNA Sequence | GAAATGTCACCACATACATTCTCACAGTAATGAACATGGTTTCTGGCCTATTTAAAGA |
| | TGGGACTATTGGAAGTGACATAAACGTGGTTGTGGTGAGCCTAATTCTTCTGGAACAA |
| | GAACCTGGAGGATTATTGATCAACCATCATGCAGACCAGTCTCTGAATAGTTTTTGTC |
| | AATGGCAGTCTGCCCTCATTGGAAAGAATGGCAAGAGACATGATCATGCCATCTTACT |
| | AACAGGATTTGATATTTGTTCTTGGAAGAATGAACCATGTGACACTCTAGGGTTTGCC |
| | CCCATCAGTGGAATGTGCTCTAAGTACCGAAGTTGTACCATCAATGAGGACACAGGAC |
| | TTGGCCTTGCCTTCACCATCGCTCATGAGTCAGGGCACAACTTTGGTATGATTCACGA |
| | CGGAGAAGGGAATCCCTGCAGAAAGGCTGAAGGCAATATCATGTCTCCCACACTGACC |
| | GGAAACAATGGAGTGTTTTCATGGTCTTCCTGCAGCCGCCAGTATCTCAAGAAATTCC |
| | TCAGCACACCTCAGGCGGGGTGTCTAGTGGATGAGCCCAAGCAAGCAGGACAGTATAA |
| | ATATCCGGACAAACTACCAGGACAGATTTATGATGCTGACACACTGTGTAAATGGCAA |
| | TTTGGAGCAAAAGCCAAGTTATGCAGCCTTGGTTTTGTGAAGGATATTTGCAAATCAC |
| | TTTGGTGCCACCGAGTAGGCCACAGGTGTGAGACCAAGTTTATGCCCGCAGCAGAAGG |
| | GACCGTTTGTGGCTTGAGTATGTGGTGTCGGCAAGGCCAGTGCGTAAAGTTTGGGGAG |
| | CTCGGGCCCCGGCCCATCCACGGCCAGTGGTCCGCCTGGTCGAAGTGGTCAGAATGTT |
| | CCCGGACATGTGGTGGAGGAGTCAAGTTCCAGGAGAGACACTGCAATAACCCCAAGCC |
| | TCAGTATGGTGGCTTGTTCTGTCCAGGTTCTAGCCGTATTTATCAGCTGTGCAATATT |
| | AACCCTTGCCTCGAG |

| | |
|---|---|
| ORF Start: at 7 | ORF Stop: at 1054 |

| | | |
|---|---|---|
| SEQ ID NO: 126 | 349 aa | MW at 38372.7kD |

| | |
|---|---|
| NOV39b, | VETLVVADKQMVEKHGKGNVTTYILTVMNMVSGLFKDGTIGSDINVVVVSLILLEQEP |
| CG110205-02 | |
| Protein Sequence | GGLLINHHADQSLNSFCQWQSALIGKNGKRHDHAILLTGFDICSWKNEPCDTLGFAPI |
| | SGMCSKYRSCTINEDTGLGLAFTIAHESGHNFGMIHDGEGNPCRKAEGNIMSPTLTGN |
| | NGVFSWSSCSRQYLKKFLSTPQAGCLVDEPKQAGQYKYPDKLPGQIYDADTLCKWQFG |
| | AKAKLCSLGFVKDICKSLWCHRVGHRCETKFMPAAEGTVCGLSMWCRQGQCVKFGELG |
| | PRPIHGQWSAWSKWSECSRTCGGGVKFQERHCNNPKPQYGGLFCPGSSRIYQLCNINP |
| | C |

| | |
|---|---|
| SEQ ID NO: 127 | 1059 bp |

| | |
|---|---|
| NOV39c, | AAGCTTGTGGAAACCCTCGTGGTGGCAGACAAGCAAATGGTGGAAAAGCATGGCAAGG |
| 207756942 DNA | |
| Sequence | GAAATGTCACCACATACATTCTCACAGTAATGAACATGGTTTCTGGCCTATTTAAAGA |
| | TGGGACTATTGGAAGTGACATAAACGTGGTTGTGGTGAGCCTAATTCTTCTGGAACAA |
| | GAACCTGGAGGATTATTGATCAACCATCATGCAGACCAGTCTCTGAATAGTTTTTGTC |
| | AATGGCAGTCTGCCCTCATTGGAAAGAATGGCAAGAGACATGATCATGCCATCTTACT |
| | AACAGGATTTGATATTTGTTCTTGGAAGAATGAACCATGTGACACTCTAGGGTTTGCC |
| | CCCATCAGTGGAATGTGCTCTAAGTACCGAAGTTGTACCATCAATGAGGACACAGGAC |
| | TTGGCCTTGCCTTCACCATCGCTCATGAGTCAGGGCACAACTTTGGTATGATTCACGA |
| | CGGACAAGGGAATCCCTGCAGAAAGGCTGAAGGCAATATCATGTCTCCCACACTGACC |
| | GGAAACAATGGAGTGTTTTCATGGTCTTCCTGCAGCCGCCAGTATCTCAAGAAATTCC |
| | TCAGCACACCTCAGGCGGGGTGTCTAGTGGATGAGCCCAAGCAAGCAGGACAGTATAA |
| | ATATCCGGACAAACTACCAGGACAGATTTATGATGCTGACACACAGTGTAAATGGCAA |
| | TTTGGAGCAAAAGCCAAGTTATGCAGCCTTGGTTTTGTGAAGGATATTTGCAAATCAC |
| | TTTGGTGCCACCGAGTAGGCCACAGGTGTGAGACCAAGTTTATGCCCGCAGCAGAAGG |
| | GACCGTTTGTGGCTTGAGTATGTGGTGTCGGCAAGGCCAGTGCGTAAAGTTTGGGGAG |
| | CTCGGGCCCCGGCCCATCCACGGCCAGTGGTCCGCCTGGTCGAAGTGGTCAGAATGTT |
| | CCCGGACATGTGGTGGAGGAGTCAAGTTCCAGGAGAGACACTGCAATAACCCCAAGCC |
| | TCAGTATGGTGGCTTATTCTGTCCAGGTTCTAGCCGTATTTATCAGCTGTGCAATATT |
| | AACCCTTGCCTCGAG |

| | |
|---|---|
| ORF Start: at 1 | ORF Stop: end of sequence |

| | | |
|---|---|---|
| SEQ ID NO: 128 | 353 aa | MW at 38871.3kD |

| | |
|---|---|
| NOV39c, | KLVETLVVADKQMVEKHGKGNVTTYILTVMNMVSGLFKDGTIGSDINVVVVSLILLEQ |
| 207756942 | |
| Protein Sequence | EPGGLLINHHADQSLNSFCQWQSALIGKNGKRHDHAILLTGFDICSWKNEPCDTLGFA |
| | PISGMCSKYRSCTINEDTGLGLAFTIAHESGHNFGMIHDGEGNPCRKAEGNIMSPTLT |
| | GNNGVFSWSSCSRQYLKKFLSTPQAGCLVDEPKQAGQYKYPDKLPGQIYDADTQCKWQ |
| | FGAKAKLCSLGFVKDICKSLWCHRVGHRCETKFMPAAEGTVCGLSMWCRQGQCVKFGE |
| | LGPRPIHGQWSAWSKWSECSRTCGGGVKFQERHCNNPKPQYGGLFCPGSSRIYQLCNI |
| | NPCLE |

| | |
|---|---|
| SEQ ID NO: 129 | 1059 bp |

| | |
|---|---|
| NOV39d, | AAGCTTGTGGAAACCCTCGTGGTGGCAGACAAGAAAATGGTGGAAAAGCATGGCAAGG |
| 207756946 DNA | |
| Sequence | GAAATGTCACCACATACATTCTCACAGTAATGAACATGGTTTCTGGCCTATTTAAAGA |
| | TGGGACTATTGGAAGTGACATAAACGTGGTTGTGGTGAGCCTAATTCTTCTGGAACAA |
| | GAACCTGGAGGATTATTGATCAACCATCATGCAGACCAGTCTCTGAATAGTTTTTGTC |
| | AATGGCAGTCTGCCCTCATTGGAAAGAATGGCAAGAGACATGATCATGCCATCTTACT |
| | AACAGGATTTGATATTTGTTCTTGGAAGAATGAACCATGTGACACTCTAGGGTTTGCC |
| | CCCATCAGTGGAATGTGCTCTAAGTACCGAAGTTGTACCATCAATGAGGACACAGGAC |
| | TTGGCCTTGCCTTCACCATCGCTCATGAGTCAGGGCACAACTTTGGTATGATTCACGA |
| | CGGAGAAGGGAATCCCTGCAGAAAGGCTGAAGGCAATATCATGTCTCCCACACTGACC |
| | GGAAACAATGGAGTGTTTTCATGGTCTTCCTGCAGCCGCCAGTATCTCAAGAAATTCC |
| | TCAGCACACCTCAGGCGGGGTGTCTAGTGGATGAGCCCAAGCAAGCAGGACAGTATAA |
| | ATATCCGGACAAACTACCAGGACAGATTTATGATGCTGACACACAGTGTAAATGGCAA |
| | TTTGGAGCAAAAGCCAAGTTATGCAGCCTTGGTTTTGTGAAGGATATTTGCAAATCAC |
| | TTTGGTGCCACCGGGTAGGCCACAGGTGTGAGACCAAGTTTATGCCCGCAGCAGAAGG |
| | GACCGTTTGTGGCTTGAGTATGTGGTGTCGGCAAGGCCAGTGCGTAAAGTTTGGGGAG |
| | CTCGGGCCCCGGCCCATCCACGGCCAGTGGTCCGCCTGGTCGAAGTGGTCAGAATGTT |
| | CCCGGACATGTGGTGGAGGAGTCAAGTTCCAGGAGAGACACTGCAATAACCCCAAGCC |
| | TCAGTATGGTGGCATATTCTGTCCAGGTTCTAGCCGTATTTATCAGCTGTGCAATATT |
| | AACCCTTGCCTCGAG |

| | |
|---|---|
| ORF Start: at 1 | ORF Stop: end of sequence |

| | | |
|---|---|---|
| SEQ ID NO: 130 | 353 aa | MW at 38871.3kD |

| | |
|---|---|
| NOV39d, | KLVETLVVADKKMVEKHGKGNVTTYILTVMNMVSGLFKDGTIGSDINVVVVSLILLEQ |
| 207756946 | |
| Protein Sequence | EPGGLLINHHADQSLNSFCQWQSALIGKNGKRHDHAILLTGFDICSWKNEPCDTLGFA |
| | PISGMCSKYRSCTINEDTGLGLAFTIAHESGHNFGMIHDGEGNPCRKAEGNIMSPTLT |
| | GNNGVFSWSSCSRQYLKKFLSTPQAGCLVDEPKQAGQYKYPDKLPGQIYDADTQCKWQ |
| | FGAKAKLCSLGFVKDICKSLWCHRVGHRCETKFMPAAEGTVCGLSMWCRQGQCVKFGE |
| | LGPRPIHGQWSAWSKWSECSRTCGGGVKFQERHCNNPKPQYGGIFCPGSSRIYQLCNI |
| | NPCLE |

| | |
|---|---|
| SEQ ID NO: 131 | 1059 bp |

| | |
|---|---|
| NOV39e, | AAGCTTGTGGAAACCCTCGTGGTGGCAGACAAGAAAATGGTGGAAAAGCATGGCAAGG |
| 207756950 DNA | |
| Sequence | GAAATGTCACCACATACATTCTCACAGTAATGAACATGGTTTCTGGCCTATTTAAAGA |
| | TGGGACTATTGGAAGTGACATAAACGTGGTTGTGGTGAGCCTAATTCTTCTGGAACAA |
| | GAACCTGGAGGATTATTGATCAACCATCATGCAGACCAGTCTCTGAATAGTTTTTGTC |
| | AATGGCAGTCTGCCCTCATTGGAA~GAATGGCAAGAGACATGATCATGCCATCTTACT |
| | AACAGGATTTGATATTTGTTCTTGGAAGAATGAACCATGTGGCACTCTAGGGTTTGCC |
| | CCCATCAGTGGAATGTGCTCTAAGTACCGAAGTTGTACCATCAATGAGGACACAGGAC |
| | TTGGCCTTGCCTTCACCATCGCTCATGAGTCAGGGCACAACTTTGGTATGATTCACGA |
| | CGGAGAAGGGAATCCCTGCAGAAAGGCTGAAGGCAATATCATGTCTCCCACACTGACC |
| | GGAAACAATGGAGTGTTTTCATGGTCTTCCTGCAGCCGCCAGTATCTCAAGAAATTCC |
| | TCAGCACACCTCAGGCGGGGTGTCTAGTGGATGAGCCCAAGCAAGCAGGACAGTATAA |
| | ATATCCGGACAAACTACCAGGACAGATTTATGATGCTGACACACAGTGTAAATGGCAA |
| | TTTGGAGCAAAAGCCA~GTTATGCAGCCTTGGTTTTGTGGAGGATATTTGCAAATCAC |
| | TTTGGTGCCACCGAGTAGGCCACAGGTGTGAGACCAAGTTTATGCCCGCAGCAGAAGG |
| | GACCGTTTGTGGCTTGAGTATGTGGTGTCGGCAAGGCCAGTGCGTAAAGTTTGGGGAG |
| | CTCGGGCCCCGGCCCATCCACGGCCAGTGGTCCGCCTGGTCGAATTGGTCAGAATGTT |
| | CCCGGACATGTGGTGGAGGAGTCAAGTTCCAGGAGAGACACTGCAATAACCCCAAGCC |
| | TCAGTATGGTGGCATATTCTGTCCAGGTTCTAGCCGTATTTATCAGCTGTGCAATATT |
| | AACCCTTGCCTCGAG |

| | |
|---|---|
| ORF Start: at 1 | ORF Stop: end of sequence |

| | | |
|---|---|---|
| SEQ ID NO: 132 | 353 aa | MW at 38800.1kD |

| | |
|---|---|
| NOV39e, | KLVETLVVADKKMVEKHGKGNVTTYILTVMMMVSGLFKDGTIGSDINVVVVSLILLEQ |
| 207756950 | |
| Protein Sequence | EPGGLLINHHADQSLNSFCQWQSALIGKNGKRHDHAILLTGFDICSWKNEPCGTLGFA |
| | PISGMCSKYRSCTINEDTGLGLAFTIAHESGHNFGMIHDGEGNPCRKAEGNIMSPTLT |
| | GNNGVFSWSSCSRQYLKKFLSTPQAGCLVDEPKQAGQYKYPDKLPGQIYDADTQCKWQ |
| | FGAKAKLCSLGFVEDICKSLWCHRVGHRCETKFMPAAEGTVCGLSMWCRQGQCVKFGE |
| | LGPRPIHGQWSAWSNWSECSRTCGGGVKFQERHCNNPKPQYGGIFCPGSSRIYQLCNI |
| | NPCLE |

| | |
|---|---|
| SEQ ID NO: 133 | 1059 bp |

| | |
|---|---|
| NOV39f, | AAGCTTGTGGAAACCCTCGTGGTGGCAGACAAGAAAATGGTGGAAAAGCATGGCAAGG |
| 207756966 DNA | |
| Sequence | GAAATGTCACCACATACATTCTCACAGTAATGAACATGGTTTCTGGCCTATTTAAAGA |
| | TGGGACTATTGGAAGTGACATAAACGTGGTTGTGGTGAGCCTAATGCTTCTGGAACAA |
| | GAACCTGGAGGATTATTGATCAACCATCATGCAGACCAGTCTCTGAATAGTTTTTGTC |
| | AATGGCAGTCTGCCCTCATTGGAAAGAATGGCAAGAGACATGATCATGCCATCTTACT |
| | AACAGGATTTGATATTTGTTCTTGGAAGAATGAACCATGTGACACTCTAGGGTTTGCC |
| | CCCATCAGTGGAATGTGCTCTAAGTACCGAAGTTGTACCATCAATGAGGACACAGGAC |
| | TTGGCCTTGCCTTCACCATCGCTCATGAGTCAGGGCACAACTTTGGTATGATTCACGA |
| | CGGAGAAGGGAATCCCTGCAGAAAGGCTGAAGGCAATATCATGTCTCCCACACTGACC |
| | GGAAACAATGGAGTGTTTTCATGGTCTTCCTGCAGCCGCCAGTATCTCAAGAAATTCC |
| | TCAGCACACCTCAGGCGGGGTGTCTAGTGGATGAGCCCAAGCAAGCAGGACAGTATAA |
| | ATATCCGGACAAACTACCAGGACAGATTTATGATGCTGACACACAGTGTAAATGGCAA |
| | TTTGGAGCAAAAGCCAAGTTATGCAGCCTTGGTTTTGTGAAGGATATTTGCAAATCAC |
| | TTTGGTGCCACCGAGTAGGCCACAGGTGTGAGACCAAGTTTATGCCCGCAGCAGAAGG |
| | GACCGTTTGTGGCTTGAGTATGTGGTGTCGGCAAGGCCAGTGCGTAAAGTTTGGGGAG |
| | CTCGGGCCCCGGCCCATCCACGGCCAGTGGTCCGCCTGGTCGAAGTGGTCAGAATGTT |
| | CCCGGACATGTGGTGGAGGAGTCAAGTTCCAGGAGAGACACTGCAATAACCCCAAGCC |
| | TCAGTATGGTGGCATATTCTGTCCAQGTTCTAGCCGTATTTATCAGCTGTGCAATATT |
| | AACCCTTGCCTCGAG |

| | |
|---|---|
| ORF Start: at 1 | ORF Stop: end of sequence |

| | | |
|---|---|---|
| SEQ ID NO: 134 | 353 aa | MW at 38889.3kD |

| | |
|---|---|
| NOV39f, | KLVETLVVADKKMVEKHGKGNVTTYILTVD4NMVSGLFKDGTIGSDINVVVSLMLLEQ |
| 207756966 | |
| Protein Sequence | EPGGLLINHHADQSLNSFCQWQSALIGKNGKRHDHAILLTGFDICSWKNEPCDTLGFA |
| | PISGMCSKYRSCTINEDTGLGLAFTIAHESGHNFGMIHDGEGNPCRKAEGNIMSPTLT |
| | GNNGVFSWSSCSRQYLKKFLSTPQAGCLVDEPKQAGQYKYPDKLPGQIYDADTQCKWQ |
| | FGAKAKLCSLGFVKDICKSLWCHRVGHRCETKFMPAAEGTVCGLSMWCRQGQCVKFGE |
| | LGPRPIHGQWSAWSKWSECSRTCGGGVKFQERHCNNPKPQYGGIFCPGSSRIYQLCNI |
| | NPCLE |

Sequence comparison of the above protein sequences yields the following sequence relationships shown in Table 39B.

TABLE 39B
Comparison of NOV39a against NOV39b through NOV39f.

| | NOV39a Residues/ | Identities/Similarities |
|---|---|---|
| Protein Sequence | Match Residues | for the Matched Region |
| NOV39b | 295 . . . 643 | 347/349 (99%) |
| | 1 . . . 349 | 348/349 (99%) |
| NOV39c | 295 . . . 645 | 349/351 (99%) |
| | 3 . . . 353 | 350/351 (99%) |
| NOV39d | 295 . . . 645 | 349/351 (99%) |
| | 3 . . . 353 | 350/351 (99%) |
| NOV39e | 295 . . . 645 | 346/351 (98%) |
| | 3 . . . 353 | 348/351 (98%) |
| NOV39f | 295 . . . 645 | 348/351 (99%) |
| | 3 . . . 353 | 350/351 (99%) |

Further analysis of the NOV39a protein yielded the following properties shown in Table 39C.

TABLE 39C
Protein Sequence Properties NOV39a

| | |
|---|---|
| PSort | 0.6400 probability located in plasma membrane; |
| analysis: | 0.4600 probability located in Golgi body; 0.3700 probability |
| | located in endoplasmic reticulum (membrane); |
| | 0.1000 probability located in endoplasmic reticulum (lumen) |
| SignalP | Cleavage site between residues 48 and 49 |
| analysis: | |

A search of the NOV39a protein against the Geneseq database, a proprietary database that contains sequences published in patents and patent publication, yielded several homologous proteins shown in Table 39D.

TABLE 39D
Geneseq Results for NOV39a

| | | NOV39a | Identities/ | |
|---|---|---|---|---|
| | | Residues/ | Similarities for | |
| Geneseq | Protein/Organism/Length [Patent | Match | the Matched | Expect |
| Identifier | #, Date] | Residues | Region | Value |
| AAU72893 | Human metalloprotease partial | 305 . . . 1162 | 856/934 (91%) | 0.0 |
| | protein sequence #5 - Homo | 1 . . . 934 | 858/934 (91%) | |
| | sapiens, 934 aa. [WO200183782- | | | |
| | A2, 08-NOV-2001] | | | |
| AAU72891 | Human metalloprotease partial | 20 . . . 1160 | 660/1238 (53%) | 0.0 |
| | protein sequence #3 - Homo | 9 . . . 1221 | 828/1238 (66%) | |
| | sapiens, 1224 aa. [WO200183782- | | | |
| | A2, 08-NOV-2001] | | | |
| AAU72890 | Human metalloprotease partial | 59 . . . 1158 | 415/1134 (36%) | 0.0 |
| | protein sequence #2 - Homo | 37 . . . 1100 | 585/1134 (50%) | |
| | sapiens, 1103 aa. [WO200183782- | | | |
| | A2, 08-NOV-2001] | | | |
| AAB74945 | Human ADAM type metal protease | 59 . . . 1158 | 413/1134 (36%) | 0.0 |
| | MDTS2 protein SEQ ID NO: 10 - | 37 . . . 1100 | 585/1134 (51%) | |
| | Homo sapiens, 1103 aa. | | | |
| | [JP2001008687-A, 16-JAN-2001] | | | |
| AAB47719 | ADAMTS-E - Homo sapiens, 1104 | 59 . . . 1158 | 412/1134 (36%) | 0.0 |
| | aa. [EP1149903-A1, 31-OCT-2001] | 37 . . . 1101 | 583/1134 (51%) | |

In a BLAST search of public sequence databases, the NOV39a protein was found to have homology to the proteins shown in the BLASTP data in Table 39E.

TABLE 39E
Public BLASTP Results for NOV39a

| | | NOV39a | Identities/ | |
|---|---|---|---|---|
| Protein | | Residues/ | Similarities for | |
| Accession | | Match | the Matched | Expect |
| Number | Protein/Organism/Length | Residues | Portion | Value |
| CAC83612 | ADAMTS18 PROTEIN - Homo | 1 . . . 1073 | 937/1081 (86%) | 0.0 |
| | sapiens (Human), 1081 aa. | 1 . . . 1064 | 965/1081 (88%) | |
| CAC86015 | METALLOPROTEASE | 20 . . . 1007 | 576/1081 (53%) | 0.0 |
| | DISINTEGRIN 16 WITH | 9 . . . 1065 | 719/1081 (66%) | |
| | THROMBOSPONDIN TYPE I | | | |
| | MOTIF - Homo sapiens (Human), | | | |
| | 1072 aa. | | | |
| Q9H324 | ADAMTS-10 precursor (EC 3.4.24.—) | 59 . . . 1158 | 412/1134 (36%) | 0.0 |
| | (A disintegrin and | 11 . . . 1074 | 584/1134 (51%) | |
| | metalloproteinase with | | | |
| | thrombospondin motifs 10) | | | |
| | (ADAM-TS 10) (ADAM-TS10) - | | | |
| | Homo sapiens (Human), 1077 aa | | | |
| | (fragment). | | | |
| CAD20434 | SEQUENCE 8 FROM PATENT | 59 . . . 1101 | 398/1074 (37%) | 0.0 |
| | WO0188156 - Homo sapiens | 37 . . . 1041 | 558/1074 (51%) | |
| | (Human), 1044 aa (fragment). | | | |
| P58397 | ADAMTS-12 precursor (EC 3.4.24.—) | 60 . . . 1058 | 375/1026 (36%) | 0.0 |
| | (A disintegrin and | 51 . . . 997 | 548/1026 (52%) | |
| | metalloproteinase with | | | |
| | thrombospondin motifs 12) | | | |
| | (ADAM-TS 12) (ADAM- TS12) - | | | |
| | Homo sapiens (Human), 1593 aa. | | | |

PFam analysis predicts that the NOV39a protein contains the domains shown in the Table 39F.

TABLE 39F
Domain Analysis of NOV39a

| | | Identities/ | |
|---|---|---|---|
| | NOV39a | Similarities | Expect |
| Pfam Domain | Match Region | for the Matched Region | Value |
| Pep_M12B_propep | 111 . . . 222 | 27/119 (23%) | 1.6e−13 |
| | | 72/119 (61%) | |
| Reprolysin | 295 . . . 498 | 66/221 (30%) | 1.1e−21 |
| | | 158/221 (71%) | |
| tsp_1 | 593 . . . 643 | 23/54 (43%) | 1.2e−12 |
| | | 36/54 (67%) | |
| tsp_1 | 879 . . . 932 | 13/60 (22%) | 0.0042 |
| | | 39/60 (65%) | |
| tsp_1 | 934 . . . 989 | 18/64 (28%) | 0.022 |
| | | 36/64 (56%) | |
| tsp_1 | 997 . . . 1056 | 18/64 (28%) | 0.015 |
| | | 39/64 (61%) | |
| tsp_1 | 1072 . . . 1118 | 14/55 (25%) | 0.0041 |
| | | 34/55 (62%) | |

### Example 40

The NOV40 clone was analyzed, and the nucleotide and encoded polypeptide sequences are shown in Table 40A.

TABLE 40A
NOV40 Sequence Analysis

| | SEQ ID NO:135 | 3107 bp |
|---|---|---|
| NOV40a | CGTGGAGGTTCTTGTCCAGGGCACATGGAGGACCGTGTGTTGCGTGACGACCTCTGGA | |
| CG110242-01 | ATCTGGCCAAGGCCACTGTCGTGTGCCGCCAGCTACAGTGTGGACGGGCTGTGGCAGC | |
| DNA Sequence | CCCAACAGGGGCTCACTTCGGGGCAGGCTCTGGGAAAATCCTGCTGGATGACGTGCAG | |
| | TGTGTGGGGAGCGAGAGCCACCTGGGGCAGTGCGTGCATGGGGGCCGGGCCAGGCACA | |
| | ACTGTGGGCACCTGGAGGATGCCAGTGTCATCTGTTCAAGGCCACTCCCCAGTGTCCC | |
| | TACTTCTTGTGCCTCCCCAGGAGCCTGGATGGAGGTGAGGCTGCTGAACGGCACAGGA | |
| | AGGTGCTCAGGCCGTGTGGAAGTTCTCGTCCAGGGCACGTGGGGGACCGTGTGTGATG | |
| | ATCTCTGGGACCTGGCTGAGGCCACTGTCGTGTGCCGCCAGCTGCAGTGTGGCCAGGC | |
| | TGTGGCAGCCCCCACAGGGGCCCACTTTCGGGCAGGCTCTGGGAAGATCTTACTGGAT | |
| | GACATGCAGTGTGTGGGCAGTGAGAGCCATCTGGGTCAATGCATGCGTGGGGACCAGG | |
| | CCAGGCACAACTGTGGGCACCTGGAGGATGCCAGTGTCATCTGCACACACCATCAGTT | |
| | ACCAGCTGCAGGAGCCTGCATGGAGGTGAGGCTGCTGAATGGCACAGGGAGGTGCTTA | |
| | GGCCGCGTGGAGGTTCTCATCCAGGGCACGTGGGGGACGGTGTGTGACCACTTCTGGA | |
| | ACCTGGCCGAGGCCGCCGTTGTGTGCCGCCAGCTGCAATGTGGCCAGGCCATGGCAGC | |
| | CCACTTCGGGGCAAGCTCTGGGAAAGTCCTGCTGGATGACATGCAGTGTGTGGGCAGC | |
| | AAGAGCCACCTGGGGCGGTGCGTGCACAGGGGCTGGGCCAGGCACAACTGTGGGCACC | |
| | TGGAGGATGCCAGTGTCATCTGTGCAGAAAAATCTCGGTGTGGTGGCATTATTACCAA | |
| | CTCATCTGGAGCGATTAGGAATCCCCCACAGAATGAAATGCATGACAACATCACTTGT | |
| | GTGTGGGAAATCAAGGCAAATGCATCTGATCATATACTGCTGGCATTTCCACATCTTC | |
| | TTGACTGCACCAATGAATATTTTGAAATCCTGGACGGTCCACCATCCTCAGTAAAGTC | |
| | ATTGGGGAGGACCTGCTCTGGCTTCCAACACACCTACATCTCCTCCTCCAGCTCAATG | |
| | ACCCTCGTGTATTTCCGAAGCTTCAACAACCTAGGAAAATGGCTATTGACGATGTATT | |
| | TCCTTGTACTTGCAGAGGCAGTGTTGCAGACCCCACATCTAATCAGTCAGTGCCCCCA | |
| | GAGGACAACAGTCACCAGATCTCTCTCTGCAGGGGACTGGCCGGAGCTGCAGCTGGTG | |
| | GGTGGCTCTGGCCGGTGCTCAGGACGCGTGGAGATTCTCCACCAGGGCGCCTGGGGCA | |
| | CCGTGTGTGATGACCTGTGGGACCTGAACGAAGCTGAGGTTGTGTGCCGGCAGCTTGG | |
| | GTGTGGTCGAGCCATGTCTGCCCTTGGAAAGGCCCACTTTGGCCCCGGCTCAGGAGAC | |
| | ATCTTCCTGGACAACCTCCAGTGCGCTGGTGTGGAGCGCTACCTGGGCCAGTGCACCC | |
| | ACTCGGGCTGGTCAGAGCACAACCATGTTTCATATTCAGGCATAATTTCCACTTCAGA | |
| | AGAAGTAACTCCTTCACCTGGTTGTGGCGGTTACCTGGATACCTTGGAAGGATCCTTC | |
| | ACCAGCCCCAATTACCCAAAGCCGCATCCTGAGCTGGCTTATTGTGTGTGGCACATAC | |
| | AAGTGGAGAAAGATTACAAGATAAAACTAAACTTCAAAGAGATTGAAATAGACAAACA | |
| | GTGCAAATTTGATTTTCTTGCCATCTATGATGGCCCCTCCACCAACTCTGGCCTGATT | |
| | GGACAAGTCTGTGGCCGTGTGACTCCCACCTTCGAATCGTCATCAAACTCTCTGACTG | |
| | TCGTGTTGTCTACAGATTATGCCAATTCTTACCGGGGATTTTCTGCTTCCTACACCTC | |
| | AATTTATGCAGAAAACATCACAGCATCTTTAACTTGCTCTTCTGACAGGATGAGAGTT | |
| | ATTATAAGCAAATCCTACCTAGAGGCTTTTAACTCTAATGGGAATAACTTGCAACTAA | |
| | AAGACCCAACTTGCAGACCAAAATTATCAAATGTTGTGGAATTTTCTGTCCCTCTTAA | |
| | TGGATGTGGTACAATCAGACAGGTAGAAGATCAGTCAATTACTTACACCAATATAATC | |
| | ACCTTTTCTGCATCCTCAACTTCTGAAGTGATCACCCGTCAGAAACAACTCCAGATTA | |
| | TTGTGAAGTGTGAAATGGGACATAATTCTACAGTGGAGATAATATACATAACAGAAGA | |
| | TGATGTAATACAAAGTCAAAATGCACTGGGCAAATATAACACCAGCATGGCTCTTTTT | |
| | GAATCCAATTCATTTGAAAAGACTATACTTGAATCACCATATTATGTGGATTTGAACC | |
| | AAACTCTTTTTGTTCAAGTTAGTCTGCACACCTCAGATCCAAATTTGGTGGTGTTTCT | |
| | TGATACCTGTAGAGCCTCTCCCACCTCTGACTTTGCATCTCCAACCTACGACCTAATC | |
| | AAGAGTGGGTGTAGTCGAGATGAAACTTGTAAGGTGTATCCCTTATTTGGACACTATG | |
| | GGAGATTCCAGTTTAATGCCTTTAAATTCTTGAGAAGTATGAGCTCTGTGTATCTGCA | |
| | GTGTAAAGTTTTGATATGTGATAGCAGTGACCACCAGTCTCGCTGCAATCAAGGTTGT | |
| | GTCTCCAGAAGCAAACGAGACATTTCTTCATATAAATGGAAAACAGATTCCATCATAG | |
| | GACCCATTCGTCTGAAAAGGGATCGAAGTGCAAGTGGCAATTCAGGATCTCAGCATGA | |
| | AACACATGCGGAAGAAACTCCAAACCAGCCTTTCAACAGTGTGCATCTGTTTTCCTTC | |
| | ATGGTTCTAGCTCTGAATGTGGTGACTGTAGCGACAATCACAGTGAGGCATTTTGTAA | |
| | ATCAACGGGCAGACTACAAATACCAGAAGCTGCAGAACTATTAACTAACAGGTCCAAC | |
| | CCTAAGTGAGACATGTTTCTCCAGGATGCCAAA | |
| | ORF Start: ATG at 25 | ORF Stop: TAA at 3058 |
| | SEQ ID NO:136 | 1011 aa MW at 110883.1 kD |
| NOV40a, | MEDRVLRDDLWNLAKATVVCRQLQCGRAVAAPTGAHFGAGSGKILLDDVQCVGSESHL | |
| CG110242-01 | GQCVHGGRARHNCGHLEDASVICSRPLPSVPTSCASPGAWMEVRLLNGTGRCSGRVEV | |
| Protein Sequence | LVQGTWGTVCDDLWDLAEATVVCRQLQCGQAVAAPTGAHFRAGSGKILLDDMQCVGSE | |
| | SHLGQCMRGDQARHNCGHLEDASVICTHHQLPAAGACMEVRLLNGTGRCLGRVEVLIQ | |
| | GTWGTVCDHFWNLAEAAVVCRQLQCGQAMAAHFGASSGKVLLDDMQCVGSKSHLGRCV | |
| | HRGWARHNCGHLEDASVICAEKSRCGGIITNSSGAIRNPPQNEMHDNITCVWEIKANA | |
| | SDHILLAFPHLLDCTNEYFEILDGPPSSVKSLGRTCSGFQHTYISSSSSMTLVYFRSF | |
| | NNLGKWLLTMYFLVLAEAVLQTPHLISQCPQRTTVTRSLSAGDWPELQLVGGSGRCSG | |
| | RVEILHQGAWGTVCDDLWDLNEAEVVCRQLGCGRAMSALGKAHFGPGSGDIFLDNLQC | |
| | AGVERYLGQCTHSGWSEHNHVSYSGIISTSEEVTPSPGCGGYLDTLEGSFTSPNYPKP | |
| | HPELAYCVWHIQVEKDYKIKLNFKEIEIDKQCKFDFLAIYDGPSTNSGLIGQVCGRVT | |
| | PTFESSSNSLTVVLSTDYANSYRGFSASYTSIYAENITASLTCSSDRMRVIISKSYLE | |
| | AFNSNGNNLQLKDPTCRPKLSNVVEFSVPLNGCGTIRQVEDQSITYTNIITFSASSTS | |
| | EVITRQKQLQIIVKCEMGHNSTVEIIYITEDDVIQSQNALGKYNTSMALFESNSFEKT | |
| | ILESPYYVDLNQTLFVQVSLHTSDPNLVVFLDTCRASPTSDFASPTYDLIKSGCSRDE | |
| | TCKVYPLFGHYGRFQFNAFKFLRSMSSVYLQCKVLICDSSDHQSRCNQGCVSRSKRDI | |
| | SSYKWKTDSIIGPIRLKRDRSASGNSGSQHETHAEETPNQPFNSVHLFSFMVLALNVV | |
| | TVATITVRHFVNQRADYKYQKLQNY | |
| | SEQ ID NO:137 | 744 bp |
| NOV40b, | GGTACCACTTGCTCTTCTGACAGGATGAGAGTTATTATAAGCAAATCCTACCTAGAGG | |
| 207728344 DNA | CTTTTAACTCTAATGGGAATAACTTGCAACTAAAAGACCCAACTTGCAGACCAAAATT | |
| Sequence | ATCAAATGTTGTGGAATTTTCTGTCCCTCTTAATGGATGTGGTACAATCAGAAAGGTA | |
| | GAAGATCAGTCAATTACTTACACCAATATAATCGCCTTTTCTGCATCCTCAACTTCTG | |
| | AAGTGATCACCCGTCAGAAACAACTCCAGATTATTGTGAAGTGTGAAATGGGACATAA | |
| | TTCTACAGTGGAGATAATATACATAACAGAAGATGATGTAATACAAAGTCAAAATGCA | |
| | CTGGGCAAATATAACACCAGCATGGCTCTTTTTGAATCCAATTCATTTGAAAAGACTA | |
| | TACTTGAATCACCATATTATGTGGATTTGAACCAAACTCTTTTTGTTCAAGTTAGTCT | |
| | GCACACCTCAGATCCAAATTTGGTGGTGTTTCTTGATACCTGTAGAGCCTCTCCCACC | |
| | TCTGACTTTGCATCTCCAACCTACGACCTAATCAAGAGTGGATGTAGTCGAGATGAAA | |
| | CTTGTAAGGTGTATCCCTTATTTGGACACTATGGGAGATTCCAGTTTAATGCCTTTAA | |
| | ATTCTTGAGAAGTATGAGCTCTGTGTATCTGCAGTGTAAAGTTTTGATATGTGATAGC | |
| | AGTGACCACCAGTCTCGCTGCAATCAAGGTTGTGTCTCCAGACTCGAG | |
| | ORF Start: at 1 | ORF Stop: end of sequence |
| | SEQ ID NO:138 | 248 aa MW at 27830.1 kD |
| NOV40b, | GTTCSSDRMRVIISKSYLEAFNSNGNNLQLKDPTCRPKLSNVVEFSVPLNGCGTIRKV | |
| 207728344 | EDQSITYTNIIAFSASSTSEVITRQKQLQIIVKCEMGHNSTVEIIYITEDDVIQSQNA | |
| Protein Sequence | LGKYNTSMALFESNSFEKTILESPYYVDLNQTLFVQVSLHTSDPNLVVFLDTCRASPT | |
| | SDFASPTYDLIKSGCSRDETCKVYPLFGHYGRFQFNAFKFLRSMSSVYLQCKVLICDS | |
| | SDHQSRCNQGCVSRLE | |
| | SEQ ID NO:139 | 744 bp |
| NOV40c, | GGTACCACTTGCTCTTCTGACAGGATGAGAGTTATTATAAGCAAATCCTACCTAGAGG | |
| 207728348 DNA | CTTTTAACTCTAATGGGAATAACTTGCAACTAAAAGACCCAACTTGCAGACCAAAATT | |
| Sequence | ATCAAATGTTGTGGAATTTTCTGTCCCTCTTAATGGATGTGGTACAATCAGAAAGGTA | |
| | GAAGATCAGTCAATTACTTACACCAATATAATCACCTTTTCTGCATCCTCAACTTCTG | |
| | AAGTGATCACCCGTCAGAAACAACTCCAGATTATTCTGAAGTGTGAAATGGGACATAA | |
| | TTCTACAGTGGAGATAATATACATAACAGAAGATGATGTAATACAAAGTCAAAATGCA | |
| | CTGGGCAAATATAACACCAGCATGGCTCTTTTTGAATCCAATTCATTTGAAAAGACTA | |
| | TACTTGAAACACCATATTATGTGGATTTGAACCAAACTCTTTTTGTTCAAGTTAGTCT | |
| | GCACACCTCAGATCCAAATTTGGTGGTGTTTCTTGATACCTGTAGAGCCTCTCCCACC | |
| | TCTGACTTTGCATCTCCAACCTACGACCTAATCAAGAGTGGATGTAGTCGAGATGAAA | |
| | CTTGTAAGGTGTATCCCTTATTTGGACACTATGGGAGATTCCAGTTTAATGCCTTTAA | |
| | ATTCTTGAGAAGTATGAGCTCTGTGTATCTGCAGTGTAAAGTTTTGATATGTGATAGC | |
| | AGTGACCACCAGTCTCGCTGCAATCAAGGTTGTGTCTCCAGACTCGAG | |
| | ORF Start: at 1 | ORF Stop: end of sequence |
| | SEQ ID NO:140 | 248 aa MW at 27888.2 kD |
| NOV40c, | GTTCSSDRMRVIISKSYLEAFNSNGNNLQLKDPTCRPKLSNVVEFSVPLNGCGTIRKV | |
| 207728348 | EDQSITYTNIITFSASSTSEVITRQKQLQIILKCEMGHNSTVEIIYITEDDVIQSQNA | |
| Protein Sequence | LGKYNTSMALFESNSFEKTILETPYYVDLNQTLFVQVSLHTSDPNLVVFLDTCRASPT | |
| | SDFASPTYDLIKSGCSRDETCKVYPLFGHYGRFQFNAFKFLRSMSSVYLQCKVLICDS | |
| | SDHQSRCNQGCVSRLE | |
| | SEQ ID NO:141 | 744 bp |
| NOV40d, | GGTACCACTTGCTCTTCTGACAGGATGAGAGTTATTATAAGCAAATCCTACCTAGAGG | |
| 207728354 DNA | CTTTTAACTCTAATGGGAATAACTTGCAACTAAAAGACCCAACTTGCAGACCAAAATT | |
| Sequence | ATCAAATGTTGTGGAATTTTCTGTCCCTCTTAATGGATGTGGTACAATCAGAAAGGTA | |
| | GAAGATCAGTCAATTACTTACACCAATATAATCACCTTTTCTGCATCCTCAACTTCTG | |
| | AAGTGATCACCCGTCAGAAACAACTCCAGATTATTGTGAAGTGTGAAATGGGACATAA | |
| | TTCTACAGTGGAGATAATATACATAACAGAAGATGATGTAATACAAAGTCAAAATGCA | |
| | CTGGGCAAATATAACACCAGCATGGCTCTTTTTGAATCCAATTCATTTGAAAAGACTA | |
| | TACTTGAATCACCATATTATGTGGATTTGAACCAAACTCTTTTTGTTCAAGTTAGTCT | |
| | GCACACCTCAGATCCAAATTTGGTGGTGTTTCTTGATACCTGTAGAGCCTCTCCCACC | |
| | TCTGACTTTGCATCTCCAACCTACGACCTAATCAAGAGTGGATGTAGTCGAGATGAAA | |
| | CTTGTAAGGTGTATCCCTTATTTGGACACTATGGGAGATTCCAGTTTAATGCCTTTAA | |
| | ATTCTTGAGAAGTATGAGCTCTGTGTATCTGCAGTGTAAAGTTTTGATATGTGATAGC | |
| | AGTGACCACCAGTCTCGCTGCAATCAAGGTTGTGTCTCCAGACTCGAG | |
| | ORF Start: at 1 | ORF Stop: end of sequence |
| | SEQ ID NO:142 | 248 aa MW at 27860.2 kD |
| NOV40d, | GTTCSSDRMRVIISKSYLEAFNSNGNNLQLKDPTCRPKLSNVVEFSVPLNGCGTIRKV | |
| 207728354 | EDQSITYTNIITFSASSTSEVITRQKQLQIIVKCEMGHNSTVEIIYITEDDVTQSQNA | |
| Protein Sequence | LGKYNTSMALFESNSFEKTILESPYYVDLNQTLFVQVSLHTSDPNLVVFLDTCRASPT | |
| | SDFASPTYDLIKSGCSRDETCKVYPLFGHYGRFQFNAFKFLRSMSSVYLQCKVLICDS | |
| | SDHQSRCNQGCVSRLE | |
| | SEQ ID NO:143 | 744 bp |
| NOV40e, | GGTACCACTTGCTCTTCTGACAGGATGAGAGTTATTATAAGCAAATCCTACCTAGAGG | |
| 207728365 DNA | CTTTTAACTCTAATGGGAATAACTTGCAACTAAAAGACCCAACTTGCAGACCAAAATT | |
| Sequence | ATCAAATGTTGTGGAATTTTCTGTCCCTCTTAATGGATGTGGTACAATCAGAAAGGTA | |
| | GAAGATCAGTCAATTACTTACACCAATATAATCACCCTTTCTGCATCCTCAACTTCTG | |
| | AAGTGATCACCCGTCAGAAACAACTCCAGATTATTGTGAAGTGTGAAATGGGACATAA | |
| | TTCTACAGTGGAGATAATATACATAACAGAAGATGATGTAATACAAAGTCAAAATGCA | |
| | CTGGGCAAATATAACACCAGCATGGCTCTTTTTGAATCCAATTCATTTGAAAAGACTA | |
| | TACTTGAATCACCATATTATGTGGATTTGAACCAAACTCTTTTTGTTCAAGTTAGTCT | |
| | GCACACCTCAGATCCAAATTTGGTGGTGTTTCTTGATACCTGTAGAGCCTCTCCCACC | |
| | TCTGACTTTGCATCTCCAACCTACGACCTAATCAAGAGTGGATGTAGTCGAGATGAAA | |
| | CTTGTAAGGTGTATCCCTTATTTGGACACTATGGGAGATTCCAGTTTAATGCCTTTAA | |
| | ATTCTTGAGAAGTATGAGCTCTGTGTATCTGCAGTGTAAAGTTTTGATATGTGATAGC | |
| | AGTGACCACCAGTCTCGCTGCAATCAAGGTTGTGTCTCCAGACTCGAG | |
| | ORF Start: at 1 | ORF Stop: end of sequence |
| | SEQ ID NO:144 | 248 aa MW at 27826.1 kD |

| | |
|---|---|
| NOV40e, | GTTCSSDRMRVIISKSYLEAFNSNGNNLQLKDPTCRPKLSNVVEFSVPLNGCGTIRKV |
| 207728365 | EDQSITYTNIITLSASSTSEVITRQKQLQIIVKCEMGHNSTVETIYITEDDVIQSQNA |
| Protein Sequence | LGKYNTSMALFESNSFEKTILESPYYVDLNQTLFVQVSLHTSDPNLVVFLDTCRASPT |
| | SDFASPTYDLIKSGCSRDETCKVYPLFGHYGRFQFNAFKFLRSMSSVYLQCKVLICDS |
| | SDHQSRCNQGCVSRLE |

Sequence comparison of the above protein sequences yields the following sequence relationships shown in Table 40B.

TABLE 40B
Comparison of NOV40a against NOV40b through NOV40e.

| | NOV40a Residues/ | Identities/Similarities |
|---|---|---|
| Protein Sequence | Match Residues | for the Matched Region |
| NOV40b | 680 . . . 923 | 242/244 (99%) |
| | 3 . . . 246 | 243/244 (99%) |
| NOV40c | 680 . . . 923 | 241/244 (98%) |
| | 3 . . . 246 | 244/244 (99%) |
| NOV40d | 680 . . . 923 | 243/244 (99%) |
| | 3 . . . 246 | 244/244 (99%) |
| NOV40e | 680 . . . 923 | 242/244 (99%) |
| | 3 . . . 246 | 243/244 (99%) |

Further analysis of the NOV40a protein yielded the following properties shown in Table 40C.

TABLE 40C
Protein Sequence Properties NOV40a

| | |
|---|---|
| PSort | 0.7000 probability located in plasma membrane; |
| analysis: | 0.5843 probability located in |
| | mitochondrial inner membrane; |
| | 0.3000 probability located in microbody |
| | (peroxisome); 0.2000 probability located |
| | in endoplasmic reticulum (membrane) |
| SignalP | No Known Signal Sequence Predicted |
| analysis: | |

A search of the NOV40a protein against the Geneseq database, a proprietary database that contains sequences published in patents and patent publication, yielded several homologous proteins shown in Table 40D.

TABLE 40D
Geneseq Results for NOV40a

| | | NOV40a | Identities/ | |
|---|---|---|---|---|
| | | Residues/ | Similarities for | |
| Geneseq | Protein/Organism/Length [Patent | Match | the Matched | Expect |
| Identifier | #, Date] | Residues | Region | Value |
| AAB80245 | Human PRO257 protein - Homo | 559 . . . 1011 | 449/456 (98%) | 0.0 |
| | sapiens, 607 aa. [WO200104311- | 152 . . . 607 | 450/456 (98%) | |
| | A1, 18-JAN-2001] | | | |
| AAU12343 | Human PRO257 polypeptide | 559 . . . 1011 | 449/456 (98%) | 0.0 |
| | sequence - Homo sapiens, 607 aa. | 152 . . . 607 | 450/456 (98%) | |
| | [WO200140466-A2, 07-JUN-2001] | | | |
| AAB07456 | Protein encoded by a novel gene | 559 . . . 1011 | 449/456 (98%) | 0.0 |
| | associated with insulin synthesis - | 130 . . . 585 | 450/456 (98%) | |
| | Homo sapiens, 585 aa. | | | |
| | [WO200040722-A2, 13-JUL-2000] | | | |
| AAY13377 | Amino acid sequence of protein | 559 . . . 1011 | 449/456 (98%) | 0.0 |
| | PRO257 - Homo sapiens, 607 aa. | 152 . . . 607 | 450/456 (98%) | |
| | [WO9914328-A2, 25-MAR-1999] | | | |
| AAY25323 | Human pancreatic PA153 consensus | 559 . . . 1011 | 449/456 (98%) | 0.0 |
| | protein - Homo sapiens, 607 aa. | 152 . . . 607 | 450/456 (98%) | |
| | [WO9931274-A2, 24-JUN-1999] | | | |

In a BLAST search of public sequence databases, the NOV40a protein was found to have homology to the proteins shown in the BLASTP data in Table 40E.

TABLE 40E
Public BLASTP Results for NOV40a

| | | NOV40a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q96DU4 | DMBT1/8KB.2 PROTEIN | 8 . . . 945 | 435/1028 (42%) | 0.0 |
| | PRECURSOR - Homo sapiens | 1406 . . . 2403 | 605/1028 (58%) | |
| | (Human), 2413 aa. | | | |
| Q9UGM3 | DMBT1 PROTOTYPE | 8 . . . 945 | 435/1028 (42%) | 0.0 |
| | PRECURSOR - Homo sapiens | 1419 . . . 2416 | 605/1028 (58%) | |
| | (Human), 2426 aa. | | | |
| Q9Y4V9 | DMBT1/6KB.1 PROTEIN | 8 . . . 945 | 435/1028 (42%) | 0.0 |
| | PRECURSOR - Homo sapiens | 778 . . . 1775 | 605/1028 (58%) | |
| | (Human), 1785 aa. | | | |
| Q9UKJ4 | GP-340 VARIANT PROTEIN - | 8 . . . 945 | 435/1028 (42%) | 0.0 |
| | Homo sapiens (Human), 2413 aa. | 1406 . . . 2403 | 605/1028 (58%) | |
| Q9Y211 | DMBT1 - Homo sapiens | 8 . . . 945 | 435/1028 (42%) | 0.0 |
| | (Human), 1785 aa. | 778 . . . 1775 | 605/1028 (58%) | |

PFam analysis predicts that the NOV40a protein contains the domains shown in the Table 40F.

TABLE 40F
Domain Analysis of NOV40a

| | | Identities/ | |
|---|---|---|---|
| | | Similarities | |
| | NOV40a | for the | |
| Pfam Domain | Match Region | Matched Region | Expect Value |
| SRCR | 1 . . . 82 | 37/114 (32%) | 2.2e−10 |
| | | 62/114 (54%) | |
| SRCR | 104 . . . 201 | 48/114 (42%) | 2.4e−34 |
| | | 78/114 (68%) | |
| SRCR | 217 . . . 310 | 43/113 (38%) | 3.2e−24 |
| | | 72/113 (64%) | |
| CUB | 315 . . . 416 | 38/118 (32%) | 2.6e−10 |
| | | 72/118 (61%) | |
| SRCR | 456 . . . 551 | 47/114 (41%) | 1.9e−28 |
| | | 78/114 (68%) | |
| CUB | 561 . . . 667 | 38/117 (32%) | 4.9e−35 |
| | | 83/117 (71%) | |
| zona_pellucida | 680 . . . 923 | 78/286 (27%) | 4.1e−40 |
| | | 184/286 (64%) | |

### Example 41

The NOV41 clone was analyzed, and the nucleotide and encoded polypeptide sequences are shown in Table 41A.

TABLE 41A
NOV41 Sequence Analysis

| | SEQ ID NO:145 | 4011 bp |
|---|---|---|
| NOV41a, | ATGCCTCTGTCCAGCCACCTGCTGCCCGCCTTGGTCCTGTTCCTGGGCCACCTGGCTG | |
| CG99598-01 | TCAACCTCCCAGCAGCAGGGTCCTCAGGCTGGGCCTGGGTCCCCAACCACTGCAGGAG | |
| DNA Sequence | CCCTGGCCAGGCCGTGTGCAACTTCGTGTGTGACTGCAGGGACTGCTCAGATGAGGCC | |
| | CAGTGTGGTTACCACGGGGCCTCGCCCACCCTGGGCGCCCCCTTCGCCTGTGACTTCG | |
| | AGCAGGACCCCTGCGGCTGGCGGGACATTAGTACCTCAGGCTACAGCTGGCTCCGAGA | |
| | CAGGGCAGGGGCCGCACTGGAGGGTCCTGGGCCTCACTCAGACCACACACTGGGCACC | |
| | GACTTGGGCTGGTACATGGCCGTTGGAACCCACCGAGGGAAAGAGGCATCCACCGCAG | |
| | CCCTGCGCTCGCCAACCCTGCGAGAGGCAGCCTCCTCTTGCAAGCTGAGGCTCTGGTA | |
| | CCACGCGGCCTCTGGAGATGTGGCTGAACTGCGGGTGGAGCTGACCCATGGCGCAGAG | |
| | ACCCTGACCCTGTGGCAGAGCACAGGGCCCTGGGGCCCTGGCTGGCAGGAGTTGGCAG | |
| | TGACCACAGGCCGCATCCGGGGTGACTTCCGAGTGACCTTCTCTGCCACCCGAAATGC | |
| | CACCCACAGGGGCGCTGTGGCTCTAGATGACCTAGAGTTCTGGGACTGTGGTCTGCCC | |
| | ACCCCCCAGGCCAACTGTCCCCCGGGACACCACCACTGCCAGAACAAGGTCTGCGTGG | |
| | AGCCCCAGCAGCTGTGCGACGGGGAAGACAACTGCGGGGACCTGTCTGATGAGAACCC | |
| | ACTCACCTGTGGCCGCCACATAGCCACCGACTTTGAGACAGGCCTGGGCCCATGGAAC | |
| | CGCTCGGAAGGCTGGTCCCGGAACCACCGCGCTGGTGGTCCTGAGCGCCCCTCCTGGC | |
| | CACGCCGTGACCACAGCCGGAACAGTGCACAGGGCTCCTTCCTGGTCTCCGTGGCCGA | |
| | GCCTGGCACCCCTGCTATACTCTCCAGCCCCGAATTCCAAGCCTCAGQCACCTCCAAC | |
| | TGCTCCGCCCCCAGCCAGCTCTTGGTTCCACAGCTGGTCTTCTATCAGTACCTGAGTG | |
| | GGTCTGAGGCTGGCTGCCTCCAGCTGTTCCTGCAGACTCTGGGGCCCGGCGCCCCCCG | |
| | GGCCCCCGTCCTGCTGCGGAGGCGCCGAGGGGAGCTGGGGACCGCCTGGGTCCGAGAC | |
| | CGTGTTGACATCCAGAGCGCCTACCCCTTCCAGATCCTCCTGGCCGGGCAGACAGGCC | |
| | CGGGGGGCGTCGTGGGTCTGGACGACCTCATCCTGTCTGACCACTGCAGACCAGTCTC | |
| | GGAGGTGTCCACCCTGCAGCCGCTGCCTCCTGGGCCCCGGGCCCCAGCCCCCCAGCCC | |
| | CTGCCGCCCAGCTCGCGGCTCCAGGATTCCTGCAAGCAGGGGCATCTTGCCTGCGGGG | |
| | ACCTGTGTGTGCCCCCGGAACAACTGTGTGACTTCGAGGAGCAGTGCGCAGGGGGCGA | |
| | GGACGAGCAGGCCTGTGGTAAAGGGGCTCAACCTCCTGCAGACCTCCTGCTGCGGAGC | |
| | CAAGGGGGCACCACAGACTTTGAGTCCCCCGAGGCTGGGGGCTGGGAGGACGCCAGCG | |
| | TGGGGCGGCTGCAGTGGCGGCGTGTCTCAGCCCAGGAGAGCCAGGGGTCCAGTGCAGC | |
| | TGCTGCTGGCTTCCTGGCACTAGTTGTGGTGGACAACGGCTCCCGGGAGCTGGCATGG | |
| | CAGGCCCTGAGCAGCAGTGCAGGCATCTGGAAGGTGGACAAGGTCCTTCTAGGGGCCC | |
| | GCCGCCGGCCCTTCCGGCTGGAGTTTGTCGGTTTGGTGGACTTGGATGGCCCTGACCA | |
| | GCAGGGAGCTGGGGTGGACAACGTGACCCTGAGGGACTGTAGCCCCACAGTGACCACC | |
| | GAGAGAGACAGAGAGGTCTCCTGTAACTTTGAGCGGGACACATGCAGCTGGTACCCAG | |
| | GCCACCTCTCAGACACACACTGGCGCTGGGTGGAGAGCCGCGGCCCTGACCACGACCA | |
| | CACCACAGGCCAAGGCCACTTTGTGCTCCTGGACCCCACAGACCCCCTGGCCTGGGGC | |
| | CACAGTGCCCACCTGCTCTCCAGGCCCCAGGTGCCAGCAGCACCCACGGAGTGTCTCA | |
| | GCTTCTGGTACCACCTCCATGGGCCCCAGATTGGGACTCTGCGCCTAGCCATGAGACG | |
| | GGAAGGGGAGGAGACACACCTGTGGTCGCGGTCAGGCACCCAGGGCAACCGCTGGCAC | |
| | GAGGCCTGGGCCACCCTTTCCCACCAGCCTGGCTCCCATGCCCAGTACCAGCTGCTGT | |
| | TCGAGGGCCTCCGGGACGGATACCACGGCACCATGGCGCTGGACGATGTGGCCGTGCG | |
| | GCCGGGCCCCTGCTGGGCCCCTAATTACTGCTCCTTTGAGGACTCAGACTGCGGCTTC | |
| | TCCCCTGGAGGCCAAGGTCTCTGGAGGCGGCAGGCCAATGCCTCGGGCCATGCTGCCT | |
| | GGGGCCCCCCAACAGACCATACCACTGAGACAGCCCAAGGGCACTACATGGTGGTGGA | |
| | CACAAGCCCAGACGCACTACCCCGGGGCCAGACGGCCTCCCTGACCTCCAAGGAGCAC | |
| | AGGCCCCTGGCCCAGCCTGCTTGTCTGACCTTCTGGTACCACGGGAGCCTCCGCAGCC | |
| | CAGGCACCCTGCGGGTCTACCTGGAGGAGCGCGGGAGGCACCAGGTGCTCAGCCTCAG | |
| | TGCCCACGGCGGGCTTGCCTGGCGCCTGGGCAGCATGGACGTGCAGGCCGAGCGAGCC | |
| | TGGAGGGTGGTGTTTGAGGCAGTGGCCGCAGGCGTGGCACACTCCTACGTGGCTCTGG | |
| | ATGATCTGCTCCTCCAGGACGGGCCCTGCCCTCAGCCAGGTTCCTGTGATTTTGAGTC | |
| | TGGCCTGTGTGGCTGGAGCCACCTGGCCTGGCCCGGCCTGGGCGGATACAGCTGGGAC | |
| | TGGGGCGGGGGAGCCACCCCCTCTCGTTACCCCCAGCCCCCTGTGGACCACACCCTGG | |
| | GCACAGAGGCAGGCCACTTTGCCTTCTTTGAAACTGGCGTGCTGGGCCCCGGGGGCCG | |
| | GGCCGCCTGGCTGCGCAGCGAGCCTCTGCCGGCCACCCCAGCCTCCTGCCTCCGCTTC | |
| | TGGTACCACATGGGTTTTCCTGAGCACTTCTACAAGGGGGAGCTGAAGGTACTGCTGC | |
| | ACAGTGCTCAGGGCCAGCTGGCTGTGTGGGGCGCAGGCGGGCATCGGCGGCACCAGTG | |
| | GCTGGAGGCCCAGGTGGAGGTAGCCAGTGCCAAGGAGTTCCAGATCGTGTTTGAAGCC | |
| | ACTCTGGGCGGCCAGCCAGCCCTGGGGCCCATTGCCCTGGATGACGTGGAGTATCTGG | |
| | CTGGGCAGCATTGCCAGCAGCCTGCCCCCAGCCCGGGGAACACAGCCGCACCCGGGTC | |
| | TGTGCCAGCTGTGGTTGGCAGTGCCCTCCTATTGCTCATGCTCCTGGTGCTGCTGGGA | |
| | CTTGGGGGACGGCGCTGGCTGCAGAAGAAGGGGAGCTGCCCCTTCCAGAGCAACACAG | |
| | AGGCCACAGCCCCTGGCTTTGACAACATCCTTTTCAATGCGGAGCCATGCGGTGTTGG | |
| | AGGGCACAGCACAGCACCCTTGCCAGCCGCTAGGCTCCCATCCGCCCTGGGGACTAAG | |
| | TCCCAGCGGAGGGCGGCAGTTGGGCACGGCTACCGCCGTCCCTCGTCTTCAGGTGCCG | |
| | TGGGGCTGACCAGTGCCCACCAACTGTCCACGCAGATGGGCGAAGAGGAAATGGCCCT | |
| | GCAGAGACCTTCAGAGCTCCCCCCGGCAGCCCACTCCCGGGCATCAGTCATGAAAATT | |
| | CACCAGCTTTCCCCACAACTAGGGGCCTGGGAGCTGAGAGCAGGACCGGACAATCTGG | |
| | CCCCAGCGCCAAGGGCAGGAACTTTTCCCAGCTTCTCTTCAGAGCTGCATCAAAGAAA | |
| | GCAGCGCCCAGTGACACCGCTCCTCTTCCTTCCACGCCTCAGGCCTCCACCCCTCACC | |
| | CTTGTATAA | |
| | ORF Start: ATG at 1 | ORF Stop: TAA at 4009 |
| | SEQ ID NO:146 | 1336 aa MW at 144032.7 kD |

| | |
|---|---|
| NOV41a, | MPLSSHLLPALVLFLGHLAVNLPAAGSSGWAWVPNHCRSPGQAVCNFVCDCRDCSDEA |
| CG99598-01 | QCGYHGASPTLGAPFACDFEQDPCGWRDISTSGYSWLRDRAGAALEGPGPHSDHTLGT |
| Protein Sequence | DLGWYMAVGTHRGKEASTAALRSPTLREAASSCKLRLWYHAASGDVAELRVELTHGAE |
| | TLTLWQSTGPWGPGWQELAVTTGRIRGDFRVTFSATRNATHRGAVALDDLEFWDCGLP |
| | TPQANCPPGHHHCQNKVCVEPQQLCDGEDNCGDLSDENPLTCGRHIATDFETGLGPWN |
| | RSEGWSRNHRAGGPERPSWPRRDHSRNSAQGSFLVSVAEPGTPAILSSPEFQASGTSN |
| | CSAPSQLLVPQLVFYQYLSGSEAGCLQLFLQTLGPGAPRAPVLLRRRRGELGTAWVRD |
| | RVDIQSAYPFQILLAGQTGPGGVVGLDDLILSDHCRPVSEVSTLQPLPPGPRAPAPQP |
| | LPPSSRLQDSCKQGHLACGDLCVPPEQLCDFEEQCAGGEDEQACGKGAQFPADLLLRS |
| | QGGTTDFESPEAGGWEDASVGRLQWRRVSAQESQGSSAAAAGFLALVVVDNGSRELAW |
| | QALSSSAGIWKVDKVLLGARRRPFRLEFVGLVDLDGPDQQGAGVDNVTLRDCSPTVTT |
| | ERDREVSCNFERDTCSWYPGHLSDTHWRWVESRGPDHDHTTGQGHFVLLDPTDPLAWG |
| | HSAHLLSRPQVPAAPTECLSFWYHLHGPQIGTLRLAMRREGEETHLWSRSGTQGNRWH |
| | EAWATLSHQPGSHAQYQLLFEGLRDGYHGTMALDDVAVRPGPCWAPNYCSFEDSDCGF |
| | SPGGQGLWRRQANASGHAAWGPPTDHTTETAQGHYMVVDTSPDALPRGQTASLTSKEH |
| | RPLAQPACLTFWYHGSLRSPGTLRVYLEERGRHQVLSLSAHGGLAWRLGSMDVQAERA |
| | WRVVFEAVAAGVAHSYVALDDLLLQDGPCPQPGSCDFESGLCGWSHLAWPGLGGYSWD |
| | WGGGATPSRYPQPPVDHTLGTEAGHFAFFETGVLGPGGPAAWLRSEPLPATPASCLRF |
| | WYHMGFPEHFYKGELKVLLHSAQGQLAVWGAGGHRRHQWLEAQVEVASAKEFQIVFEA |
| | TLGGQPALGPIALDDVEYLAGQHCQQPAPSPGNTAAPGSVPAVVGSALLLLMLLVLLG |
| | LGGRRWLQKKGSCPFQSNTEATAPGFDNILFNAEPCGVGGHSTAPLPAARLPSALGTK |
| | SQRPAAVGHGYRRPSSSGAVGLTSAHQLSTQMGEEEMALQRPSELPPAAHSRASVMKI |
| | HQLSPQLGAWELRAGPDNLAPAPRAGTFPSFSSELHQRKQRPVTPLLFLPRLRPPPLT |
| | LV |

Further analysis of the NOV41a protein yielded the following properties shown in Table 41B.

TABLE 41B
Protein Sequence Properties NOV41a

| | |
|---|---|
| PSort | 0.4600 probability located in plasma membrane; |
| analysis: | 0.2464 probability located in |
| | microbody (peroxisome); 0.1000 probability |
| | located in endoplasmic reticulum |
| | (membrane); 0.1000 probability located in |
| | endoplasmic reticulum (lumen) |
| SignalP | Cleavage site between residues 20 and 21 |
| analysis: | |

A search of the NOV41a protein against the Geneseq database, a proprietary database that contains sequences published in patents and patent publication, yielded several homologous proteins shown in Table 41C.

TABLE 41C
Geneseq Results for NOV41a

| | | NOV41a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAE17494 | Human secretion and trafficking | 1 . . . 1194 | 1159/1238 (93%) | 0.0 |
| | protein-3 (SAT-3) - Homo sapiens, | 1 . . . 1205 | 1160/1238 (93%) | |
| | 1217 aa. [WO200202610-A2, 10-JAN-2002] | | | |
| AAU29282 | Human PRO polypeptide sequence | 1 . . . 1194 | 1093/1195 (91%) | 0.0 |
| | #259 - Homo sapiens, 1137 aa. | 1 . . . 1125 | 1099/1195 (91%) | |
| | [WO200168848-A2, 20-SEP-2001] | | | |
| AAB42780 | Human ORFX ORF2544 | 161 . . . 620 | 372/475 (78%) | 0.0 |
| | polypeptide sequence SEQ ID | 45 . . . 453 | 379/475 (79%) | |
| | NO: 5088 - Homo sapiens, 465 aa. | | | |
| | [WO200058473-A2, 05-OCT-2000] | | | |
| AAB01432 | Human TANGO 239 (form 2) - | 574 . . . 957 | 110/406 (27%) | 4e−29 |
| | Homo sapiens, 686 aa. | 270 . . . 664 | 178/406 (43%) | |
| | [WO200039284-A1, 06-JUL-2000] | | | |
| ABB53298 | Human polypeptide #38 - Homo | 574 . . . 957 | 109/406 (26%) | 8e−28 |
| | sapiens, 686 aa. [WO200181363- | 270 . . . 664 | 176/406 (42%) | |
| | A1, 01-NOV-2001] | | | |

In a BLAST search of public sequence databases, the NOV41a protein was found to have homology to the proteins shown in the BLASTP data in Table 41D.

TABLE 41D
Public BLASTP Results for NOV41a

| | | NOV41a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q63191 | Apical endosomal glycoprotein | 1 . . . 1194 | 848/1245 (68%) | 0.0 |
| | precursor - Rattus norvegicus | 1 . . . 1204 | 944/1245 (75%) | |
| | (Rat), 1216 aa. | | | |
| Q91641 | Thyroid hormone-induced protein | 573 . . . 957 | 118/415 (28%) | 1e−28 |
| | B precursor - Xenopus laevis | 270 . . . 667 | 187/415 (44%) | |
| | (African clawed frog), 688 aa. | | | |
| O88799 | Zonadhesin precursor - Mus | 631 . . . 1109 | 141/518 (27%) | 3e−27 |
| | musculus (Mouse), 5376 aa. | 30 . . . 525 | 221/518 (42%) | |
| Q99ND0 | ZAN (ZONADHESIN) - Mus | 631 . . . 1109 | 141/518 (27%) | 6e−27 |
| | musculus (Mouse), 5374 aa. | 30 . . . 525 | 221/518 (42%) | |
| Q9BZ83 | ZONADHESIN VARIANT 6 - | 636 . . . 1109 | 143/522 (27%) | 8e−20 |
| | Homo sapiens (Human), 2721 aa. | 29 . . . 519 | 209/522 (39%) | |

PFam analysis predicts that the NOV41a protein contains the domains shown in the Table 41E.

TABLE 41E
Domain Analysis of NOV41a

| | | Identities/ | |
|---|---|---|---|
| | | Similarities | |
| | NOV41a | for the | |
| Pfam Domain | Match Region | Matched Region | Expect Value |
| MAM | 75 . . . 231 | 57/174 (33%) | 1.4e−44 |
| | | 122/174 (70%) | |
| ldl_recept_a | 236 . . . 276 | 13/43 (30%) | 3.3e−09 |
| | | 29/43 (67%) | |
| MAM | 280 . . . 443 | 48/188 (26%) | 7.2e−22 |
| | | 122/188 (65%) | |
| ldl_recept_a | 473 . . . 510 | 12/43 (28%) | 0.2 |
| | | 26/43 (60%) | |
| MAM | 493 . . . 634 | 33/174 (19%) | 0.0004 |
| | | 86/174 (49%) | |
| MAM | 646 . . . 799 | 68/173 (39%) | 6.7e−54 |
| | | 132/173 (76%) | |
| MAM | 803 . . . 959 | 67/173 (39%) | 2.5e−47 |
| | | 119/173 (69%) | |
| MAM | 963 . . . 1128 | 62/176 (35%) | 6.4e−56 |
| | | 137/176 (78%) | |

### Example B: Sequencing Methodology and Identification of NOVX Clones

1. GeneCalling™ Technology: This is a proprietary method of performing differential gene expression profiling between two or more samples developed at CuraGen and described by Shimkets, et al., “Gene expression analysis by transcript profiling coupled to a gene database query” Nature Biotechnology 17:198-803 (1999). cDNA was derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then digested with up to as many as 120 pairs of restriction enzymes and pairs of linker-adaptors specific for each pair of restriction enzymes were ligated to the appropriate end. The restriction digestion generates a mixture of unique cDNA gene fragments. Limited PCR amplification is performed with primers homologous to the linker adapter sequence where one primer is biotinylated and the other is fluorescently labeled. The doubly labeled material is isolated and the fluorescently labeled single strand is resolved by capillary gel electrophoresis. A computer algorithm compares the electropherograms from an experimental and control group for each of the restriction digestions. This and additional sequence-derived information is used to predict the identity of each differentially expressed gene fragment using a variety of genetic databases. The identity of the gene fragment is confirmed by additional, gene-specific competitive PCR or by isolation and sequencing of the gene fragment.

2. SeqCalling™ Technology: cDNA was derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then sequenced using CuraGen's proprietary SeqCalling technology. Sequence traces were evaluated manually and edited for corrections if appropriate. cDNA sequences from all samples were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

3. PathCalling™ Technology:

The NOVX nucleic acid sequences are derived by laboratory screening of cDNA library by the two-hybrid approach. cDNA fragments covering either the full length of the DNA sequence, or part of the sequence, or both, are sequenced. In silico prediction was based on sequences available in CuraGen Corporation's proprietary sequence databases or in the public human sequence databases, and provided either the full length DNA sequence, or some portion thereof.

The laboratory screening was performed using the methods summarized below:

cDNA libraries were derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then directionally cloned into the appropriate two-hybrid vector (Gal4-activation domain (Gal4-AD) fusion). Such cDNA libraries as well as commercially available cDNA libraries from Clontech (Palo Alto, Calif.) were then transferred from E.coli into a CuraGen Corporation proprietary yeast strain (disclosed in U.S. Pat. Nos. 6,057,101 and 6,083,693, incorporated herein by reference in their entireties).

Gal4-binding domain (Gal4-BD) fusions of a CuraGen Corportion proprietary library of human sequences was used to screen multiple Gal4-AD fusion cDNA libraries resulting in the selection of yeast hybrid diploids in each of which the Gal4-AD fusion contains an individual cDNA. Each sample was amplified using the polymerase chain reaction (PCR) using non-specific primers at the cDNA insert boundaries. Such PCR product was sequenced; sequence traces were evaluated manually and edited for corrections if appropriate. cDNA sequences from all samples were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

Physical clone: the cDNA fragment derived by the screening procedure, covering the entire open reading frame is, as a recombinant DNA, cloned into pACT2 plasmid (Clontech) used to make the cDNA library. The recombinant plasmid is inserted into the host and selected by the yeast hybrid diploid generated during the screening procedure by the mating of both CuraGen Corporation proprietary yeast strains N106′ and YULH (U.S. Pat. Nos. 6,057,101 and 6,083,693).

4. RACE: Techniques based on the polymerase chain reaction such as rapid amplification of cDNA ends (RACE), were used to isolate or complete the predicted sequence of the cDNA of the invention. Usually multiple clones were sequenced from one or more human samples to derive the sequences for fragments. Various human tissue samples from different donors were used for the RACE reaction. The sequences derived from these procedures were included in the SeqCalling Assembly process described in preceding paragraphs.

5. Exon Linking: The NOVX target sequences identified in the present invention were subjected to the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The PCR product derived from exon linking was cloned into the pCR2.1 vector from Invitrogen. The resulting bacterial clone has an insert covering the entire open reading frame cloned into the pCR2.1 vector. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported herein.

6. Physical Clone: Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

The PCR product derived by exon linking, covering the entire open reading frame, was cloned into the pCR2.1 vector from Invitrogen to provide clones used for expression and screening purposes.

### Example C: Quantitative Expression Analysis of Clones in Various Cells and Tissues

The quantitative expression of various clones was assessed using microtiter plates containing RNA samples from a variety of normal and pathology-derived cells, cell lines and tissues using real time quantitative PCR (RTQ PCR). RTQ PCR was performed on an Applied Biosystems ABI PRISM® 7700 or an ABI PRISM® 7900 HT Sequence Detection System. Various collections of samples are assembled on the plates, and referred to as Panel 1 (containing normal tissues and cancer cell lines), Panel 2 (containing samples derived from tissues from normal and cancer sources), Panel 3 (containing cancer cell lines), Panel 4 (containing cells and cell lines from normal tissues and cells related to inflammatory conditions), Panel 5D/5I (containing human tissues and cell lines with an emphasis on metabolic diseases), AI_comprehensive_panel (containing normal tissue and samples from autoimmune diseases), Panel CNSD.01 (containing central nervous system samples from normal and diseased brains) and CNS_neurodegeneration_panel (containing samples from normal and Alzheimer's diseased brains).

RNA integrity from all samples is controlled for quality by visual assessment of agarose gel electropherograms using 28S and 18S ribosomal RNA staining intensity ratio as a guide (2:1 to 2.5:1 28s:18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

First, the RNA samples were normalized to reference nucleic acids such as constitutively expressed genes (for example, β-actin and GAPDH). Normalized RNA (5 ul) was converted to cDNA and analyzed by RTQ-PCR using One Step RT-PCR Master Mix Reagents (Applied Biosystems; Catalog No. 4309169) and gene-specific primers according to the manufacturer's instructions.

In other cases, non-normalized RNA samples were converted to single strand cDNA (sscDNA) using Superscript II (Invitrogen Corporation; Catalog No. 18064-147) and random hexamers according to the manufacturer's instructions. Reactions containing up to 10 μg of total RNA were performed in a volume of 20 μl and incubated for 60 minutes at 42° C. This reaction can be scaled up to 50 μg of total RNA in a final volume of 100 μl. sscDNA samples are then normalized to reference nucleic acids as described previously, using 1×TaqMan® Universal Master mix (Applied Biosystems; catalog No. 4324020), following the manufacturer's instructions.

Probes and primers were designed for each assay according to Applied Biosystems Primer Express Software package (version I for Apple Computer's Macintosh Power PC) or a similar algorithm using the target sequence as input. Default settings were used for reaction conditions and the following parameters were set before selecting primers: primer concentration=250 nM, primer melting temperature (Tm) range=58°-60° C., primer optimal Tm=59° C., maximum primer difference=2° C., probe does not have 5′G, probe Tm must be 10° C. greater than primer Tm, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe, respectively. Their final concentrations were: forward and reverse primers, 900 nM each, and probe, 200 nM.

PCR conditions: When working with RNA samples, normalized RNA from each tissue and each cell line was spotted in each well of either a 96 well or a 384-well PCR plate (Applied Biosystems). PCR cocktails included either a single gene specific probe and primers set, or two multiplexed probe and primers sets (a set specific for the target clone and another gene-specific set multiplexed with the target probe). PCR reactions were set up using TaqMan® One-Step RT-PCR Master Mix (Applied Biosystems, Catalog No. 4313803) following manufacturer's instructions. Reverse transcription was performed at 48° C. for 30 minutes followed by amplification/PCR cycles as follows: 95° C. 10 min, then 40 cycles of 95° C. for 15 seconds, 60° C. for 1 minute. Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100.

When working with sscDNA samples, normalized sscDNA was used as described previously for RNA samples. PCR reactions containing one or two sets of probe and primers were set up as described previously, using 1×TaqMan® Universal Master mix (Applied Biosystems; catalog No. 4324020), following the manufacturer's instructions. PCR amplification was performed as follows: 95° C. 10 min, then 40 cycles of 95° C. for 15 seconds, 60° C. for 1 minute. Results were analyzed and processed as described previously.

Panels 1, 1.1, 1.2, and 1.3D

The plates for Panels 1, 1.1, 1.2 and 1.3D include 2 control wells (genomic DNA control and chemistry control) and 94 wells containing cDNA from various samples. The samples in these panels are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in these panels are widely available through the American Type Culture Collection (ATCC), a repository for cultured cell lines, and were cultured using the conditions recommended by the ATCC. The normal tissues found on these panels are comprised of samples derived from all major organ systems from single adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose.

In the results for Panels 1, 1.1, 1.2 and 1.3D, the following abbreviations are used:

ca.=carcinoma,

*=established from metastasis,

met=metastasis,

s cell var=small cell variant,

non-s=non-sm=non-small,

squam=squamous,

pl. eff=pl effusion=pleural effusion,

glio=glioma,

astro=astrocytoma, and

neuro=neuroblastoma.

General_Screening_Panel_v1.4

The plates for Panel 1.4 include 2 control wells (genomic DNA control and chemistry control) and 94 wells containing cDNA from various samples. The samples in Panel 1.4 are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in Panel 1.4 are widely available through the American Type Culture Collection (ATCC), a repository for cultured cell lines, and were cultured using the conditions recommended by the ATCC. The normal tissues found on Panel 1.4 are comprised of pools of samples derived from all major organ systems from 2 to 5 different adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose. Abbreviations are as described for Panels 1, 1.1, 1.2, and 1.3D.

Panels 2D and 2.2

The plates for Panels 2D and 2.2 generally include 2 control wells and 94 test samples composed of RNA or cDNA isolated from human tissue procured by surgeons working in close cooperation with the National Cancer Institute's Cooperative Human Tissue Network (CHTN) or the National Disease Research Initiative (NDRI). The tissues are derived from human malignancies and in cases where indicated many malignant tissues have “matched margins” obtained from noncancerous tissue just adjacent to the tumor. These are termed normal adjacent tissues and are denoted “NAT” in the results below. The tumor tissue and the “matched margins” are evaluated by two independent pathologists (the surgical pathologists and again by a pathologist at NDRI or CHTN). This analysis provides a gross histopathological assessment of tumor differentiation grade. Moreover, most samples include the original surgical pathology report that provides information regarding the clinical stage of the patient. These matched margins are taken from the tissue surrounding (i.e. immediately proximal) to the zone of surgery (designated “NAT”, for normal adjacent tissue, in Table RR). In addition, RNA and cDNA samples were obtained from various human tissues derived from autopsies performed on elderly people or sudden death victims (accidents, etc.). These tissues were ascertained to be free of disease and were purchased from various commercial sources such as Clontech (Palo Alto, Calif.), Research Genetics, and Invitrogen. General oncology screening panel_v_{—}2.4 is an updated version of Panel 2D.

Panel 3D

The plates of Panel 3D are comprised of 94 cDNA samples and two control samples. Specifically, 92 of these samples are derived from cultured human cancer cell lines, 2 samples of human primary cerebellar tissue and 2 controls. The human cell lines are generally obtained from ATCC (American Type Culture Collection), NCI or the German tumor cell bank and fall into the following tissue groups: Squamous cell carcinoma of the tongue, breast cancer, prostate cancer, melanoma, epidermoid carcinoma, sarcomas, bladder carcinomas, pancreatic cancers, kidney cancers, leukemias/lymphomas, ovarian/uterine/cervical, gastric, colon, lung and CNS cancer cell lines. In addition, there are two independent samples of cerebellum. These cells are all cultured under standard recommended conditions and RNA extracted using the standard procedures. The cell lines in panel 3D and 1.3D are of the most common cell lines used in the scientific literature. Oncology_cell_line_screening_panel_v3.2 is an updated version of Panel 3. The Cell Lines in panel 3D, 1.3D and oncology_cell_line_screening_panel_v3.2 are of the most common cell lines used in the scientific literature.

Panels 4D, 4R, and 4.1D

Panel 4 includes samples on a 96 well plate (2 control wells, 94 test samples) composed of RNA (Panel 4R) or cDNA (Panels 4D/4.1D) isolated from various human cell lines or tissues related to inflammatory conditions. Total RNA from control normal tissues such as colon and lung (Stratagene, La Jolla, Calif.) and thymus and kidney (Clontech) was employed. Total RNA from liver tissue from cirrhosis patients and kidney from lupus patients was obtained from BioChain (Biochain Institute, Inc., Hayward, Calif.). Intestinal tissue for RNA preparation from patients diagnosed as having Crohn's disease and ulcerative colitis was obtained from the National Disease Research Interchange (NDRI) (Philadelphia, Pa.).

Astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, human umbilical vein endothelial cells were all purchased from Clonetics (Walkersville, Md.) and grown in the media supplied for these cell types by Clonetics. These primary cell types were activated with various cytokines or combinations of cytokines for 6 and/or 12-14 hours, as indicated. The following cytokines were used; IL-1 beta at approximately 1-5 ng/ml, TNF alpha at approximately 5-10 ng/ml, IFN gamma at approximately 20-50 ng/ml, IL-4 at approximately 5-10 ng/ml, IL-9 at approximately 5-10 ng/ml, IL-13 at approximately 5-10 ng/ml. Endothelial cells were sometimes starved for various times by culture in the basal media from Clonetics with 0.1% serum.

Mononuclear cells were prepared from blood of employees at CuraGen Corporation, using Ficoll. LAK cells were prepared from these cells by culture in DMEM 5% FCS (Hyclone), 100 μM non essential amino acids (Gibco/Life Technologies, Rockville, Md.), 1 mM sodium pyruvate (Gibco), mercaptoethanol 5.5×10⁻⁵M (Gibco), and 10 mM Hepes (Gibco) and Interleukin 2 for 4-6 days. Cells were then either activated with 10-20 ng/ml PMA and 1-2 μg/ml ionomycin, IL-12 at 5-10 ng/ml, IFN gamma at 20-50 ng/ml and IL-18 at 5-10 ng/ml for 6 hours. In some cases, mononuclear cells were cultured for 4-5 days in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) with PHA (phytohemagglutinin) or PWM (pokeweed mitogen) at approximately 5 g/ml. Samples were taken at 24, 48 and 72 hours for RNA preparation. MLR (mixed lymphocyte reaction) samples were obtained by taking blood from two donors, isolating the mononuclear cells using Ficoll and mixing the isolated mononuclear cells 1:1 at a final concentration of approximately 2×10⁶ cells/ml in DMEM 5% FCS (Hyclone), 100 μM non essential amino acids (Gibco), 1 mM sodium pyruvate (Gibco), mercaptoethanol (5.5×10⁻⁵M) (Gibco), and 10 mM Hepes (Gibco). The MLR was cultured and samples taken at various time points ranging from 1-7 days for RNA preparation.

Monocytes were isolated from mononuclear cells using CD14 Miltenyi Beads, +ve VS selection columns and a Vario Magnet according to the manufacturer's instructions. Monocytes were differentiated into dendritic cells by culture in DMEM 5% fetal calf serum (FCS) (Hyclone, Logan, Utah), 100 μM non essential amino acids (Gibco), 1 mM sodium pyruvate (Gibco), mercaptoethanol 5.5×10⁻⁵M (Gibco), and 10 mM Hepes (Gibco), 50 ng/ml GMCSF and 5 ng/ml IL-4 for 5-7 days. Macrophages were prepared by culture of monocytes for 5-7 days in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and 10% AB Human Serum or MCSF at approximately 50 ng/ml. Monocytes, macrophages and dendritic cells were stimulated for 6 and 12-14 hours with lipopolysaccharide (LPS) at 100 ng/ml. Dendritic cells were also stimulated with anti-CD40 monoclonal antibody (Pharmingen) at 10 μg/ml for 6 and 12-14 hours.

CD4 lymphocytes, CD8 lymphocytes and NK cells were also isolated from mononuclear cells using CD4, CD8 and CD56 Miltenyi beads, positive VS selection columns and a Vario Magnet according to the manufacturer's instructions. CD45RA and CD45RO CD4 lymphocytes were isolated by depleting mononuclear cells of CD8, CD56, CD14 and CD19 cells using CD8, CD56, CD14 and CD19 Miltenyi beads and positive selection. CD45RO beads were then used to isolate the CD45RO CD4 lymphocytes with the remaining cells being CD45RA CD4 lymphocytes. CD45RA CD4, CD45RO CD4 and CD8 lymphocytes were placed in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and plated at 10⁶ cells/ml onto Falcon 6 well tissue culture plates that had been coated overnight with 0.5 μg/ml anti-CD28 (Pharmingen) and 3 ug/ml anti-CD3 (OKT3, ATCC) in PBS. After 6 and 24 hours, the cells were harvested for RNA preparation. To prepare chronically activated CD8 lymphocytes, we activated the isolated CD8 lymphocytes for 4 days on anti-CD28 and anti-CD3 coated plates and then harvested the cells and expanded them in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and IL-2. The expanded CD8 cells were then activated again with plate bound anti-CD3 and anti-CD28 for 4 days and expanded as before. RNA was isolated 6 and 24 hours after the second activation and after 4 days of the second expansion culture. The isolated NK cells were cultured in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and IL-2 for 4-6 days before RNA was prepared.

To obtain B cells, tonsils were procured from NDRI. The tonsil was cut up with sterile dissecting scissors and then passed through a sieve. Tonsil cells were then spun down and resupended at 10⁶ cells/ml in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco). To activate the cells, we used PWM at 5 μg/ml or anti-CD40 (Pharmingen) at approximately 10 μg/ml and IL-4 at 5-10 ng/ml. Cells were harvested for RNA preparation at 24, 48 and 72 hours.

To prepare the primary and secondary Th1/Th2 and Tr1 cells, six-well Falcon plates were coated overnight with 10 μg/ml anti-CD28 (Pharmingen) and 2 μg/ml OKT3 (ATCC), and then washed twice with PBS. Umbilical cord blood CD4 lymphocytes (Poietic Systems, German Town, Md.) were cultured at 10⁵-10⁶ cells/ml in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and IL-2 (4 ng/ml). IL-12 (5 ng/ml) and anti-IL4 (1 μg/ml) were used to direct to Th1, while IL-4 (5 ng/ml) and anti-IFN gamma (1 μg/ml) were used to direct to Th2 and IL-10 at 5 ng/ml was used to direct to Tr1. After 4-5 days, the activated Th1, Th2 and Tr1 lymphocytes were washed once in DMEM and expanded for 4-7 days in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and IL-2 (1 ng/ml). Following this, the activated Th1, Th2 and Tr1 lymphocytes were re-stimulated for 5 days with anti-CD28/OKT3 and cytokines as described above, but with the addition of anti-CD95L (1 μg/ml) to prevent apoptosis. After 4-5 days, the Th1, Th2 and Tr1 lymphocytes were washed and then expanded again with IL-2 for 4-7 days. Activated Th1 and Th2 lymphocytes were maintained in this way for a maximum of three cycles. RNA was prepared from primary and secondary Th1, Th2 and Tr1 after 6 and 24 hours following the second and third activations with plate bound anti-CD3 and anti-CD28 mAbs and 4 days into the second and third expansion cultures in Interleukin 2.

The following leukocyte cells lines were obtained from the ATCC: Ramos, EOL-1, KU-812. EOL cells were further differentiated by culture in 0.1 mM dbcAMP at 5×10⁵ cells/ml for 8 days, changing the media every 3 days and adjusting the cell concentration to 5×10⁵ cells/ml. For the culture of these cells, we used DMEM or RPMI (as recommended by the ATCC), with the addition of 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco). RNA was either prepared from resting cells or cells activated with PMA at 10 ng/ml and ionomycin at 1 μg/ml for 6 and 14 hours. Keratinocyte line CCD106 and an airway epithelial tumor line NCI-H292 were also obtained from the ATCC. Both were cultured in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco). CCD1106 cells were activated for 6 and 14 hours with approximately 5 ng/ml TNF alpha and 1 ng/ml IL-1 beta, while NCI-H292 cells were activated for 6 and 14 hours with the following cytokines: 5 ng/ml IL-4, 5 ng/ml IL-9, 5 ng/ml IL-13 and 25 ng/ml IFN gamma.

For these cell lines and blood cells, RNA was prepared by lysing approximately 10⁷ cells/ml using Trizol (Gibco BRL). Briefly, 1/10 volume of bromochloropropane (Molecular Research Corporation) was added to the RNA sample, vortexed and after 10 minutes at room temperature, the tubes were spun at 14,000 rpm in a Sorvall SS34 rotor. The aqueous phase was removed and placed in a 15 ml Falcon Tube. An equal volume of isopropanol was added and left at −20° C. overnight. The precipitated RNA was spun down at 9,000 rpm for 15 min in a Sorvall SS34 rotor and washed in 70% ethanol. The pellet was redissolved in 300 μl of RNAse-free water and 35 μl buffer (Promega) 5 μl DTT, 7 μl RNAsin and 8 μl DNAse were added. The tube was incubated at 37° C. for 30 minutes to remove contaminating genomic DNA, extracted once with phenol chloroform and re-precipitated with 1/10 volume of 3M sodium acetate and 2 volumes of 100% ethanol. The RNA was spun down and placed in RNAse free water. RNA was stored at −80° C.

AI_Comprehensive Panel_v1.0

The plates for AI_comprehensive panel_v1.0 include two control wells and 89 test samples comprised of cDNA isolated from surgical and postmortem human tissues obtained from the Backus Hospital and Clinomics (Frederick, Md.,). Total RNA was extracted from tissue samples from the Backus Hospital in the Facility at CuraGen. Total RNA from other tissues was obtained from Clinomics.

Joint tissues including synovial fluid, synovium, bone and cartilage were obtained from patients undergoing total knee or hip replacement surgery at the Backus Hospital. Tissue samples were immediately snap frozen in liquid nitrogen to ensure that isolated RNA was of optimal quality and not degraded. Additional samples of osteoarthritis and rheumatoid arthritis joint tissues were obtained from Clinomics. Normal control tissues were supplied by Clinomics and were obtained during autopsy of trauma victims.

Surgical specimens of psoriatic tissues and adjacent matched tissues were provided as total RNA by Clinomics. Two male and two female patients were selected between the ages of 25 and 47. None of the patients were taking prescription drugs at the time samples were isolated.

Surgical specimens of diseased colon from patients with ulcerative colitis and Crohns disease and adjacent matched tissues were obtained from Clinomics. Bowel tissue from three female and three male Crohn's patients between the ages of 41-69 were used. Two patients were not on prescription medication while the others were taking dexamethasone, phenobarbital, or tylenol. Ulcerative colitis tissue was from three male and four female patients. Four of the patients were taking lebvid and two were on phenobarbital.

Total RNA from post mortem lung tissue from trauma victims with no disease or with emphysema, asthma or COPD was purchased from Clinomics. Emphysema patients ranged in age from 40-70 and all were smokers, this age range was chosen to focus on patients with cigarette-linked emphysema and to avoid those patients with alpha-1 anti-trypsin deficiencies. Asthma patients ranged in age from 36-75, and excluded smokers to prevent those patients that could also have COPD. COPD patients ranged in age from 35-80 and included both smokers and non-smokers. Most patients were taking corticosteroids, and bronchodilators.

In the labels employed to identify tissues in the AI_comprehensive panel_v1.0 panel, the following abbreviations are used:

AI=Autoimmunity

Syn=Synovial

Normal=No apparent disease

Rep22/Rep20=individual patients

RA=Rheumatoid arthritis

Backus=From Backus Hospital

OA=Osteoarthritis

(SS)(BA)(MF)=Individual patients

Adj=Adjacent tissue

Match control=adjacent tissues

-M=Male

-F=Female

COPD=Chronic obstructive pulmonary disease

Panels 5D and 5I

The plates for Panel 5D and 5I include two control wells and a variety of cDNAs isolated from human tissues and cell lines with an emphasis on metabolic diseases. Metabolic tissues were obtained from patients enrolled in the Gestational Diabetes study. Cells were obtained during different stages in the differentiation of adipocytes from human mesenchymal stem cells. Human pancreatic islets were also obtained.

In the Gestational Diabetes study subjects are young (18-40 years), otherwise healthy women with and without gestational diabetes undergoing routine (elective) Caesarean section. After delivery of the infant, when the surgical incisions were being repaired/closed, the obstetrician removed a small sample (<1 cc) of the exposed metabolic tissues during the closure of each surgical level. The biopsy material was rinsed in sterile saline, blotted and fast frozen within 5 minutes from the time of removal. The tissue was then flash frozen in liquid nitrogen and stored, individually, in sterile screw-top tubes and kept on dry ice for shipment to or to be picked up by CuraGen. The metabolic tissues of interest include uterine wall (smooth muscle), visceral adipose, skeletal muscle (rectus) and subcutaneous adipose. Patient descriptions are as follows:

Patient 2 Diabetic Hispanic, overweight, not on insulin

Patient 7-9 Nondiabetic Caucasian and obese (BMI>30)

Patient 10 Diabetic Hispanic, overweight, on insulin

Patient 11 Nondiabetic African American and overweight

Patient 12 Diabetic Hispanic on insulin

Adipocyte differentiation was induced in donor progenitor cells obtained from Osirus (a division of Clonetics/BioWhittaker) in triplicate, except for Donor 3U which had only two replicates. Scientists at Clonetics isolated, grew and differentiated human mesenchymal stem cells (HuMSCs) for CuraGen based on the published protocol found in Mark F. Pittenger, et al., Multilineage Potential of Adult Human Mesenchymal Stem Cells Science Apr. 2, 1999: 143-147. Clonetics provided Trizol lysates or frozen pellets suitable for mRNA isolation and ds cDNA production. A general description of each donor is as follows:

Donor 2 and 3 U: Mesenchymal Stem cells, Undifferentiated Adipose

Donor 2 and 3 AM: Adipose, AdiposeMidway Differentiated

Donor 2 and 3 AD: Adipose, Adipose Differentiated

Human cell lines were generally obtained from ATCC (American Type Culture Collection), NCI or the German tumor cell bank and fall into the following tissue groups: kidney proximal convoluted tubule, uterine smooth muscle cells, small intestine, liver HepG2 cancer cells, heart primary stromal cells, and adrenal cortical adenoma cells. These cells are all cultured under standard recommended conditions and RNA extracted using the standard procedures. All samples were processed at CuraGen to produce single stranded cDNA.

Panel 5I contains all samples previously described with the addition of pancreatic islets from a 58 year old female patient obtained from the Diabetes Research Institute at the University of Miami School of Medicine. Islet tissue was processed to total RNA at an outside source and delivered to CuraGen for addition to panel 5I.

In the labels employed to identify tissues in the 5D and 5I panels, the following abbreviations are used:

GO Adipose=Greater Omentum Adipose

SK=Skeletal Muscle

UT=Uterus

PL=Placenta

AD=Adipose Differentiated

AM=Adipose Midway Differentiated

U=Undifferentiated Stem Cells

Panel CNSD.01

The plates for Panel CNSD.01 include two control wells and 94 test samples comprised of cDNA isolated from postmortem human brain tissue obtained from the Harvard Brain Tissue Resource Center. Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains two brains from each of the following diagnoses: Alzheimer's disease, Parkinson's disease, Huntington's disease, Progressive Supernuclear Palsy, Depression, and “Normal controls”. Within each of these brains, the following regions are represented: cingulate gyrus, temporal pole, globus palladus, substantia nigra, Brodman Area 4 (primary motor strip), Brodman Area 7 (parietal cortex), Brodman Area 9 (prefrontal cortex), and Brodman area 17 (occipital cortex). Not all brain regions are represented in all cases; e.g., Huntington's disease is characterized in part by neurodegeneration in the globus palladus, thus this region is impossible to obtain from confirmed Huntington's cases. Likewise Parkinson's disease is characterized by degeneration of the substantia nigra making this region more difficult to obtain. Normal control brains were examined for neuropathology and found to be free of any pathology consistent with neurodegeneration.

In the labels employed to identify tissues in the CNS panel, the following abbreviations are used:

PSP=Progressive supranuclear palsy

Sub Nigra=Substantia nigra

Glob Palladus=Globus palladus

Temp Pole=Temporal pole

Cing Gyr=Cingulate gyrus

BA 4=Brodman Area 4

Panel CNS_Neurodegeneration_V1.0

The plates for Panel CNS_Neurodegeneration_V1.0 include two control wells and 47 test samples comprised of cDNA isolated from postmortem human brain tissue obtained from the Harvard Brain Tissue Resource Center (McLean Hospital) and the Human Brain and Spinal Fluid Resource Center (VA Greater Los Angeles Healthcare System). Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains six brains from Alzheimer's disease (AD) patients, and eight brains from “Normal controls” who showed no evidence of dementia prior to death. The eight normal control brains are divided into two categories: Controls with no dementia and no Alzheimer's like pathology (Controls) and controls with no dementia but evidence of severe Alzheimer's like pathology, (specifically senile plaque load rated as level 3 on a scale of 0-3; 0=no evidence of plaques, 3=severe AD senile plaque load). Within each of these brains, the following regions are represented: hippocampus, temporal cortex (Brodman Area 21), parietal cortex (Brodman area 7), and occipital cortex (Brodman area 17). These regions were chosen to encompass all levels of neurodegeneration in AD. The hippocampus is a region of early and severe neuronal loss in AD; the temporal cortex is known to show neurodegeneration in AD after the hippocampus; the parietal cortex shows moderate neuronal death in the late stages of the disease; the occipital cortex is spared in AD and therefore acts as a “control” region within AD patients. Not all brain regions are represented in all cases.

In the labels employed to identify tissues in the CNS_Neurodegeneration_V1.0 panel, the following abbreviations are used:

AD=Alzheimer's disease brain; patient was demented and showed AD-like pathology upon autopsy

Control=Control brains; patient not demented, showing no neuropathology

Control (Path)=Control brains; pateint not demented but showing sever AD-like pathology

SupTemporal Ctx=Superior Temporal Cortex

Inf Temporal Ctx=Inferior Temporal Cortex

A. CG100689-01: LRR Protein

Expression of gene CG100689-01 was assessed using the primer-probe set Ag4186, described in Table AA. Results of the RTQ-PCR runs are shown in Tables AB and AC.

TABLE AA
Probe Name Ag4186

| | | | Start | SEQ ID |
|---|---|---|---|---|
| Primers | Sequences | Length | Position | No |
| Forward | 5′-ccagagtgttctgctctttgag-3′ | 22 | 1941 | 147 |
| Probe | TET-5′-tgctcttttatcagccagacttgaaa-3′-TAMRA | 26 | 1964 | 148 |
| Reverse | 5′-gagagtttcgtgagggtgaag-3′ | 21 | 1998 | 149 |

TABLE AB
General_screening_panel_v1.4

| | Rel. Exp. (%) | | Rel. Exp. (%) |
|---|---|---|---|
| | Ag4186, Run | | Ag4186, Run |
| Tissue Name | 221154078 | Tissue Name | 221154078 |
| Adipose | 0.0 | Renal ca. TK-10 | 0.0 |
| Melanoma* | 0.0 | Bladder | 0.0 |
| Hs688(A).T | | | |
| Melanoma* | 0.0 | Gastric ca. (liver met.) | 0.0 |
| Hs688(B).T | | NCI-N87 | |
| Melanoma* M14 | 2.3 | Gastric ca. KATO III | 15.9 |
| Melanoma* LOXIMVI | 0.0 | Colon ca. SW-948 | 0.0 |
| Melanoma* SK-MEL-5 | 0.0 | Colon ca. SW480 | 12.3 |
| Squamous Cell | 2.2 | Colon ca.* (SW480 met) | 0.0 |
| carcinoma SCC-4 | | SW620 | |
| Testis Pool | 100.0 | Colon ca. HT29 | 0.0 |
| Prostate ca.* (bone met) | 0.0 | Colon ca. HCT-116 | 28.3 |
| PC-3 | | | |
| Prostate Pool | 0.0 | Colon ca. CaCo-2 | 3.0 |
| Placenta | 0.0 | Colon cancer tissue | 0.0 |
| Uterus Pool | 0.0 | Colon ca. SW1116 | 0.0 |
| Ovarian ca. OVCAR-3 | 0.0 | Colon ca. Colo-205 | 0.0 |
| Ovarian ca. SK-OV-3 | 0.0 | Colon ca. SW-48 | 0.0 |
| Ovarian ca. OVCAR-4 | 30.1 | Colon Pool | 1.7 |
| Ovarian ca. OVCAR-5 | 0.0 | Small Intestine Pool | 1.7 |
| Ovarian ca. IGROV-1 | 0.0 | Stomach Pool | 0.0 |
| Ovarian ca. OVCAR-8 | 0.0 | Bone Marrow Pool | 0.0 |
| Ovary | 0.0 | Fetal Heart | 0.0 |
| Breast ca. MCF-7 | 1.7 | Heart Pool | 1.3 |
| Breast ca. MDA-MB- | 0.0 | Lymph Node Pool | 3.2 |
| 231 | | | |
| Breast ca. BT 549 | 0.0 | Fetal Skeletal Muscle | 0.0 |
| Breast ca. T47D | 0.0 | Skeletal Muscle Pool | 0.0 |
| Breast ca. MDA-N | 0.0 | Spleen Pool | 12.2 |
| Breast Pool | 0.0 | Thymus pool | 0.0 |
| Trachea | 1.7 | CNS cancer (glio/astro) | 0.0 |
| | | U87-MG | |
| Lung | 0.0 | CNS cancer (glio/astro) U- | 0.0 |
| | | 118-MG | |
| Fetal Lung | 2.6 | CNS cancer (neuro; met) | 0.0 |
| | | SK-N-AS | |
| Lung ca. NCI-N417 | 0.0 | CNS cancer (astro) SF-539 | 0.0 |
| Lung ca. LX-1 | 0.0 | CNS cancer (astro) SNB- | 0.0 |
| | | 75 | |
| Lung ca. NCI-H146 | 34.6 | CNS cancer (glio) SNB-19 | 1.6 |
| Lung ca. SHP-77 | 10.5 | CNS cancer (glio) SF-295 | 0.0 |
| Lung ca. A549 | 0.0 | Brain (Amygdala) Pool | 5.5 |
| Lung ca. NCI-H526 | 0.0 | Brain (cerebellum) | 4.8 |
| Lung ca. NCI-H23 | 2.4 | Brain (fetal) | 0.0 |
| Lung ca. NCI-H460 | 0.0 | Brain (Hippocampus) Pool | 9.4 |
| Lung ca. HOP-62 | 0.0 | Cerebral Cortex Pool | 0.0 |
| Lung ca. NCI-H522 | 0.0 | Brain (Substantia nigra) | 0.0 |
| | | Pool | |
| Liver | 0.0 | Brain (Thalamus) Pool | 4.4 |
| Fetal Liver | 1.7 | Brain (whole) | 0.0 |
| Liver ca. HepG2 | 0.0 | Spinal Cord Pool | 2.7 |
| Kidney Pool | 2.6 | Adrenal Gland | 0.0 |
| Fetal Kidney | 0.0 | Pituitary gland Pool | 0.0 |
| Renal ca. 786-0 | 0.0 | Salivary Gland | 0.0 |
| Renal ca. A498 | 0.0 | Thyroid (female) | 0.0 |
| Renal ca. ACHN | 0.0 | Pancreatic ca. CAPAN2 | 0.0 |
| Renal ca. UO-31 | 0.0 | Pancreas Pool | 2.4 |

TABLE AC
Panel 4.1D

| | Rel. Exp. (%) | | Rel. Exp. (%) |
|---|---|---|---|
| | Ag4186, Run | | Ag4186, Run |
| Tissue Name | 182086756 | Tissue Name | 182086756 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC | 0.0 |
| | | none | |
| Primary Th1 act | 0.0 | Lung Microvascular EC | 0.0 |
| | | TNFalpha + IL-1 beta | |
| Primary Th2 act | 0.0 | Microvascular Dermal EC | 0.0 |
| | | none | |
| Primary Tr1 act | 0.0 | Microsvasular Dermal EC | 0.0 |
| | | TNFalpha + IL-1beta | |
| Primary Th1 rest | 0.5 | Bronchial epithelium | 0.0 |
| | | TNFalpha + IL1beta | |
| Primary Th2 rest | 0.0 | Small airway epithelium | 0.0 |
| | | none | |
| Primary Tr1 rest | 0.0 | Small airway epithelium | 0.0 |
| | | TNFalpha + IL-1beta | |
| CD45RA CD4 | 0.0 | Coronery artery SMC rest | 0.0 |
| lymphocyte act | | | |
| CD45RO CD4 | 0.5 | Coronery artery SMC | 0.0 |
| lymphocyte act | | TNFalpha + IL-1beta | |
| CD8 lymphocyte act | 0.0 | Astrocytes rest | 0.0 |
| Secondary CD8 | 0.0 | Astrocytes TNFalpha + | 0.0 |
| lymphocyte rest | | IL-1beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CCD1106 (Keratinocytes) | 0.0 |
| CD95 CH11 | | none | |
| LAK cells rest | 0.0 | CCD1106 (Keratinocytes) | 0.0 |
| | | TNFalpha + IL-1beta | |
| LAK cells IL-2 | 0.0 | Liver cirrhosis | 0.4 |
| LAK cells IL-2 + IL-12 | 0.0 | NCI-H292 none | 0.0 |
| LAK cells IL-2 + IFN | 0.0 | NCI-H292 IL-4 | 0.0 |
| gamma | | | |
| LAK cells IL-2 + IL-18 | 0.0 | NCI-H292 IL-9 | 0.0 |
| LAK cells | 0.0 | NCI-H292 IL-13 | 0.0 |
| PMA/ionomycin | | | |
| NK Cells IL-2 rest | 0.0 | NCI-H292 IFN gamma | 0.0 |
| Two Way MLR 3 day | 0.0 | HPAEC none | 0.0 |
| Two Way MLR 5 day | 0.0 | HPAEC TNF alpha + IL-1 | 0.0 |
| | | beta | |
| Two Way MLR 7 day | 0.0 | Lung fibroblast none | 0.0 |
| PBMC rest | 0.0 | Lung fibroblast TNF alpha + | 0.4 |
| | | IL-1 beta | |
| PBMC PWM | 0.0 | Lung fibroblast IL-4 | 0.0 |
| PBMC PHA-L | 0.0 | Lung fibroblast IL-9 | 0.0 |
| Ramos (B cell) none | 0.4 | Lung fibroblast IL-13 | 0.0 |
| Ramos (B cell) | 0.5 | Lung fibroblast IFN | 1.1 |
| ionomycin | | gamma | |
| B lymphocytes PWM | 0.0 | Dermal fibroblast | 0.0 |
| | | CCD1070 rest | |
| B lymphocytes CD40L | 16.5 | Dermal fibroblast | 0.0 |
| and IL-4 | | CCD1070 TNF alpha | |
| EOL-1 dbcAMP | 1.0 | Dermal fibroblast | 0.0 |
| | | CCD1070 IL-1 beta | |
| EOL-1 dbcAMP | 3.8 | Dermal fibroblast IFN | 3.2 |
| PMA/ionomycin | | gamma | |
| Dendritic cells none | 0.0 | Dermal fibroblast IL-4 | 0.0 |
| Dendritic cells LPS | 0.0 | Dermal Fibroblasts rest | 1.0 |
| Dendritic cells anti- | 0.0 | Neutrophils TNFa + LPS | 0.0 |
| CD40 | | | |
| Monocytes rest | 0.0 | Neutrophils rest | 2.3 |
| Monocytes LPS | 0.0 | Colon | 1.4 |
| Macrophages rest | 0.4 | Lung | 1.6 |
| Macrophages LPS | 0.0 | Thymus | 19.6 |
| HUVEC none | 0.6 | Kidney | 100.0 |
| HUVEC starved | 0.0 | | |

General_screening_panel_v1.4 Summary: Ag4186 Expression of this gene is restricted to the testis (CT=33.7). Thus, expression of this gene could be used to differentiate between this sample and other samples on this panel and as a marker of testicular tissue. Therapeutic modulation of the expression or function of this gene may be useful in the treatment of male infertility and hypogonadism.

Panel 4.1D Summary: Ag4186 Expression of this gene is restricted to the kidney, thymus, and activated B lymphocytes (CTs=30-33). Thus, expression of this gene could be used to differentiate between the kidney derived sample and other samples on this panel and as a marker of kidney tissue. Therapeutic modulation of the expression or function of this gene could modulate kidney function and be important in the treatment of inflammatory or autoimmune diseases that affect the kidney, including lupus and glomerulonephritis.

B. CG100760-01: LRR Protein

Expression of gene CG100760-01 was assessed using the primer-probe set Ag4192, described in Table BA. Results of the RTQ-PCR runs are shown in Tables BB, BC and BD.

TABLE BA
Probe Name Ag4192

| | | | Start | SEQ ID |
|---|---|---|---|---|
| Primers | Sequences | Length | Position | No |
| Forward | 5′-tattcttttgccgagcaaca-3′ | 20 | 882 | 150 |
| Probe | TET-5′-caagttcatacacttgaacgtccagg-3′-TAMRA | 26 | 918 | 151 |
| Reverse | 5′-aatgcaatggctgtacaaaact-3 | 22 | 944 | 152 |

TABLE BB
General_screening_panel_v1.4

| | Rel. Exp. (%) | | Rel. Exp. (%) |
|---|---|---|---|
| | Ag4192, Run | | Ag4192, Run |
| Tissue Name | 221157609 | Tissue Name | 221157609 |
| Adipose | 0.0 | Renal ca. TK-10 | 0.0 |
| Melanoma* | 0.2 | Bladder | 0.0 |
| Hs688(A).T | | | |
| Melanoma* | 0.0 | Gastric ca. (liver met.) | 0.0 |
| Hs688(B).T | | NCI-N87 | |
| Melanoma* M14 | 3.3 | Gastric ca. KATO III | 4.8 |
| Melanoma* | 0.0 | Colon ca. SW-948 | 1.0 |
| LOXIMVI | | | |
| Melanoma* SK- | 2.8 | Colon ca. SW480 | 47.3 |
| MEL-5 | | | |
| Squamous cell | 0.0 | Colon ca.* (SW480 | 5.7 |
| carcinoma SCC-4 | | met) SW620 | |
| Testis Pool | 4.7 | Colon ca. HT29 | 1.0 |
| Prostate ca.* (bone | 7.8 | Colon ca. HCT-116 | 5.3 |
| met) PC-3 | | | |
| Prostate Pool | 0.0 | Colon ca. CaCo-2 | 3.7 |
| Placenta | 0.0 | Colon cancer tissue | 0.1 |
| Uterus Pool | 0.0 | Colon ca. SW1116 | 0.0 |
| Ovarian ca. | 0.3 | Colon ca. Colo-205 | 0.0 |
| OVCAR-3 | | | |
| Ovarian ca. SK-OV- | 0.0 | Colon ca. SW-48 | 0.0 |
| 3 | | | |
| Ovarian ca. | 1.4 | Colon Pool | 0.1 |
| OVCAR-4 | | | |
| Ovarian ca. | 0.2 | Small Intestine Pool | 0.8 |
| OVCAR-5 | | | |
| Ovarian ca. IGROV- | 0.0 | Stomach Pool | 0.4 |
| 1 | | | |
| Ovarian ca. | 1.3 | Bone Marrow Pool | 0.0 |
| OVCAR-8 | | | |
| Ovary | 3.1 | Fetal Heart | 0.0 |
| Breast ca. MCF-7 | 0.5 | Heart Pool | 0.3 |
| Breast ca. MDA- | 1.2 | Lymph Node Pool | 0.7 |
| MB-231 | | | |
| Breast ca. BT 549 | 0.1 | Fetal Skeletal Muscle | 0.0 |
| Breast ca. T47D | 0.0 | Skeletal Muscle Pool | 0.0 |
| Breast ca. MDA-N | 7.5 | Spleen Pool | 0.6 |
| Breast Pool | 0.0 | Thymus Pool | 0.8 |
| Trachea | 0.6 | CNS cancer (glio/astro) | 12.8 |
| | | U87-MG | |
| Lung | 0.2 | CNS cancer (glio/astro) | 15.2 |
| | | U-118-MG | |
| Fetal Lung | 0.0 | CNS cancer | 0.5 |
| | | (neuro; met) SK-N-AS | |
| Lung ca. NCI-N417 | 0.7 | CNS cancer (astro) SF- | 0.3 |
| | | 539 | |
| Lung ca. LX-1 | 0.6 | CNS cancer (astro) | 0.9 |
| | | SNB-75 | |
| Lung ca. NCI-H146 | 7.7 | CNS cancer (glio) | 0.3 |
| | | SNB-19 | |
| Lung ca. SHP-77 | 100.0 | CNS cancer (glio) SF- | 14.4 |
| | | 295 | |
| Lung ca. A549 | 0.0 | Brain (Amygdala) Pool | 0.0 |
| Lung ca. NCI-H526 | 33.0 | Brain (cerebellum) | 0.3 |
| Lung ca. NCI-H23 | 1.5 | Brain (fetal) | 0.0 |
| Lung ca. NCI-H460 | 0.0 | Brain (Hippocampus) | 0.1 |
| | | Pool | |
| Lung ca. HOP-62 | 0.0 | Cerebral Cortex Pool | 0.0 |
| Lung ca. NCI-H522 | 4.8 | Brain (Substantia nigra) | 0.0 |
| | | Pool | |
| Liver | 0.0 | Brain (Thalamus) Pool | 0.0 |
| Fetal Liver | 0.0 | Brain (whole) | 0.0 |
| Liver ca. HepG2 | 0.8 | Spinal Cord Pool | 0.0 |
| Kidney Pool | 0.3 | Adrenal Gland | 0.0 |
| Fetal Kidney | 2.9 | Pituitary gland Pool | 0.1 |
| Renal ca. 786-0 | 0.0 | Salivary Gland | 0.2 |
| Renal ca. A498 | 0.6 | Thyroid (female) | 0.6 |
| Renal ca. ACHN | 0.0 | Pancreatic ca. | 0.3 |
| | | CAPAN2 | |
| Renal ca. UO-31 | 0.0 | Pancreas Pool | 0.0 |

TABLE BC
Panel 4.1D

| | Rel. Exp. (%) | | Rel. Exp. (%) |
|---|---|---|---|
| | Ag4192, Run | | Ag4192, Run |
| Tissue Name | 175226746 | Tissue Name | 175226746 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC | 0.0 |
| | | none | |
| Primary Th1 act | 0.0 | Lung Microvascular EC | 0.0 |
| | | TNFalpha + IL-1 beta | |
| Primary Th2 act | 0.0 | Microvascular Dermal EC | 0.0 |
| | | none | |
| Primary Tr1 act | 0.0 | Microsvasular Dermal EC | 0.0 |
| | | TNFalpha + IL-1beta | |
| Primary Th1 rest | 0.0 | Bronchial epithelium | 0.0 |
| | | TNFalpha + IL1beta | |
| Primary Th2 rest | 0.0 | Small airway epithelium | 0.0 |
| | | none | |
| Primary Tr1 rest | 0.0 | Small airway epithelium | 0.0 |
| | | TNFalpha + IL-1beta | |
| CD45RA CD4 | 0.9 | Coronery artery SMC rest | 0.0 |
| lymphocyte act | | | |
| CD45RO CD4 | 2.9 | Coronery artery SMC | 0.0 |
| lymphocyte act | | TNFalpha + IL-1beta | |
| CD8 lymphocyte act | 0.0 | Astrocytes rest | 0.0 |
| Secondary CD8 | 2.1 | Astrocytes TNFalpha + | 0.0 |
| lymphocyte rest | | IL-1beta | |
| Secondary CD8 | 0.4 | KU-812 (Basophil) rest | 0.0 |
| lymphocyte act | | | |
| CD4 lymphocyte none | 0.0 | KU-812 (Basophil) | 1.0 |
| | | PMA/ionomycin | |
| 2ry Th1/Th2/Tr1_anti- | 0.0 | CCD1106 (Keratinocytes) | 0.0 |
| CD95 CH11 | | none | |
| LAK cells rest | 0.0 | CCD1106 (Keratinocytes) | 0.2 |
| | | TNFalpha + IL-1beta | |
| LAK cells IL-2 | 0.0 | Liver cirrhosis | 0.0 |
| LAK cells IL-2 + IL-12 | 0.0 | NCI-H292 none | 0.0 |
| LAK cells IL-2 + IFN | 0.9 | NCI-H292 IL-4 | 0.0 |
| gamma | | | |
| LAK cells IL-2 + IL-18 | 0.9 | NCI-H292 IL-9 | 0.9 |
| LAK cells | 0.0 | NCI-H292 IL-13 | 0.0 |
| PMA/ionomycin | | | |
| NK Cells IL-2 rest | 0.0 | NCI-H292 IFN gamma | 0.0 |
| Two Way MLR 3 day | 0.0 | HPAEC none | 0.0 |
| Two Way MLR 5 day | 0.0 | HPAEC TNF alpha + IL-1 | 0.0 |
| | | beta | |
| Two Way MLR 7 day | 0.0 | Lung fibroblast none | 0.0 |
| PBMC rest | 0.0 | Lung fibroblast TNF alpha + | 0.6 |
| | | IL-1 beta | |
| PBMC PWM | 0.0 | Lung fibroblast IL-4 | 0.0 |
| PBMC PHA-L | 2.1 | Lung fibroblast IL-9 | 0.0 |
| Ramos (B cell) none | 58.6 | Lung fibroblast IL-13 | 0.0 |
| Ramos (B cell) | 100.0 | Lung fibroblast IFN | 0.0 |
| ionomycin | | gamma | |
| B lymphocytes PWM | 2.6 | Dermal fibroblast | 0.0 |
| | | CCD1070 rest | |
| B lymphocytes CD40L | 4.7 | Dermal fibroblast | 0.0 |
| and IL-4 | | CCD1070 TNF alpha | |
| EOL-1 dbcAMP | 0.3 | Dermal fibroblast | 0.0 |
| | | CCD1070 IL-1 beta | |
| EOL-1 dbcAMP | 0.0 | Dermal fibroblast IFN | 0.0 |
| PMA/ionomycin | | gamma | |
| Dendritic cells none | 0.0 | Dermal fibroblast IL-4 | 0.0 |
| Dendritic cells LPS | 0.0 | Dermal Fibroblasts rest | 0.0 |
| Dendritic cells anti- | 0.0 | Neutrophils TNFa + LPS | 0.0 |
| CD40 | | | |
| Monocytes rest | 0.0 | Neutrophils rest | 0.0 |
| Monocytes LPS | 0.0 | Colon | 0.9 |
| Macrophages rest | 0.0 | Lung | 0.8 |
| Macrophages LPS | 0.0 | Thymus | 7.5 |
| HUVEC none | 0.0 | Kidney | 36.1 |
| HUVEC starved | 0.0 | | |

TABLE BD
general oncology screening panel_v_2.4

| | Rel. Exp. (%) | | Rel. Exp. (%) |
|---|---|---|---|
| | Ag4192, Run | | Ag4192, Run |
| Tissue Name | 268689533 | Tissue Name | 268689533 |
| Colon cancer 1 | 0.0 | Bladder cancer | 0.0 |
| | | NAT 2 | |
| Colon cancer | 0.8 | Bladder cancer | 0.0 |
| NAT 1 | | NAT 3 | |
| Colon cancer 2 | 0.0 | Bladder cancer | 0.0 |
| | | NAT 4 | |
| Colon cancer | 2.0 | Adenocarcinoma of | 4.7 |
| NAT 2 | | the prostate 1 | |
| Colon cancer 3 | 3.3 | Adenocarcinoma of | 0.0 |
| | | the prostate 2 | |
| Colon cancer | 2.4 | Adenocarcinoma of | 1.3 |
| NAT 3 | | the prostate 3 | |
| Colon malignant | 0.0 | Adenocarcinoma of | 0.0 |
| cancer 4 | | the prostate 4 | |
| Colon normal | 0.0 | Prostate cancer | 0.0 |
| adjacent tissue | | NAT 5 | |
| 4 | | | |
| Lung cancer 1 | 0.0 | Adenocarcinoma of | 1.8 |
| | | the prostate 6 | |
| Lung NAT 1 | 0.0 | Adenocarcinoma of | 0.0 |
| | | the prostate 7 | |
| Lung cancer 2 | 6.7 | Adenocarcinoma of | 1.2 |
| | | the prostate 8 | |
| Lung NAT 2 | 0.0 | Adenocarcinoma of | 0.0 |
| | | the prostate 9 | |
| Squamous cell | 4.3 | Prostate cancer | 0.0 |
| carcinoma 3 | | NAT 10 | |
| Lung NAT 3 | 0.0 | Kidney cancer 1 | 1.8 |
| metastatic | 5.0 | Kidney NAT 1 | 3.2 |
| melanoma 1 | | | |
| Melanoma 2 | 0.0 | Kidney cancer 2 | 100.0 |
| Melanoma 3 | 0.0 | Kidney NAT 2 | 10.7 |
| metastatic | 2.4 | Kidney cancer 3 | 0.9 |
| melanoma 4 | | | |
| metastatic | 20.0 | Kidney NAT 3 | 2.1 |
| melanoma 5 | | | |
| Bladder cancer | 0.0 | Kidney cancer 4 | 12.2 |
| 1 | | | |
| Bladder cancer | 0.0 | Kidney NAT 4 | 5.4 |
| NAT 1 | | | |
| Bladder cancer | 0.0 | | |
| 2 | | | |

General_screening_panel_v1.4 Summary: Ag4192 Highest expression of the CG100760-01 is seen in a lung cancer cell line (CT=29.6). Moderate levels of expression are also seen in a cluster of cell lines derived from lung, colon, and brain cancers. Thus, expression of this gene could be used to differentiate the lung cancer cell line sample from other samples on this panel and as a marker of lung, colon and brain cancer. Furthermore, this restricted pattern of expression suggests that therapeutic modulation of the expression or function of this gene may be useful in the treatment of these cancers.

Panel 4.1D Summary: Ag4192 Expression of this gene is limited to a few samples on this panel, with highest expression of the CG100760-01 gene in Ramos B cells stimulated with ionomycin (CT=30.4). Lower but still significant levels of expression are seen in untreated Ramos B cells, activated B lymphocytes, kidney and thymus. B cells represent a principle component of immunity and contribute to the immune response in a number of important functional roles, including antibody production. Production of antibodies against self-antigens is a major component in autoimmune disorders. Since B cells play an important role in autoimmunity, inflammatory processes and inflammatory cascades, therapeutic modulation of this gene product may reduce or eliminate the symptoms of patients suffering from asthma, allergies, chronic obstructive pulmonary disease, emphysema, Crohn's disease, ulcerative colitis, rheumatoid arthritis, psoriasis, osteoarthritis, systemic lupus erythematosus and other autoimmune disorders.

general oncology screening panel_v_{—}2.4 Summary: Ag4192 Expression of the CG100760-01 gene is limited in kidney cancer (CT=32) and melanoma on this panel. This expression in cancer derived samples is consistent with expression seen in Panel 1.4. Thus, expression of this gene could be used to differentiate the kidney cancer sample from other samples on this panel and as a marker of kidney cancer. Furthermore, therapeutic modulation of the expression or function of this gene may be useful in the treatment of kidney cancer.

C. CG101068-01: Claudin-9

Expression of gene CG101068-01 was assessed using the primer-probe set Ag4202, described in Table CA. Results of the RTQ-PCR runs are shown in Table CB.

TABLE CA
Probe Name Ag4202

| | | | Start | SEQ ID |
|---|---|---|---|---|
| Primers | Sequences | Length | Position | No |
| Forward | 5′-tataactccttgccatgcaaac-3′ | 22 | 1296 | 153 |
| Probe | TET-5′-tcaagaggccaatatattcctggcca-3′-TAMRA | 26 | 1320 | 154 |
| Reverse | 5′-gcatttgcatggctctaagtt-3′ | 21 | 1370 | 155 |

TABLE CB
General_screening_panel_v1.4

| | Rel. Exp. (%) | | Rel. Exp. (%) |
|---|---|---|---|
| | Ag4202, Run | | Ag4202, Run |
| Tissue Name | 221178754 | Tissue Name | 221178754 |
| Adipose | 0.4 | Renal ca. TK-10 | 0.3 |
| Melanoma* | 0.0 | Bladder | 9.2 |
| Hs688(A).T | | | |
| Melanoma* | 0.0 | Gastric ca. (liver met.) | 47.6 |
| Hs688(B).T | | NCI-N87 | |
| Melanoma* M14 | 0.0 | Gastric ca. KATO III | 100.0 |
| Melanoma* | 0.0 | Colon ca. SW-948 | 0.9 |
| LOXIMVI | | | |
| Melanoma* SK- | 0.7 | Colon ca. SW480 | 7.4 |
| MEL-5 | | | |
| Squamous cell | 0.0 | Colon ca.* (SW480 | 0.0 |
| carcinoma SCC-4 | | met) SW620 | |
| Testis Pool | 0.0 | Colon ca. HT29 | 0.0 |
| Prostate ca.* (bone | 0.0 | Colon ca. HCT-116 | 2.1 |
| met) PC-3 | | | |
| Prostate Pool | 0.0 | Colon ca. CaCo-2 | 14.6 |
| Placenta | 3.6 | Colon cancer tissue | 7.5 |
| Uterus Pool | 0.0 | Colon ca. SW1116 | 0.0 |
| Ovarian ca. | 0.7 | Colon ca. Colo-205 | 0.0 |
| OVCAR-3 | | | |
| Ovarian ca. SK-OV- | 6.7 | Colon ca. SW-48 | 0.5 |
| 3 | | | |
| Ovarian ca. | 0.0 | Colon Pool | 0.0 |
| OVCAR-4 | | | |
| Ovarian ca. | 12.9 | Small Intestine Pool | 0.0 |
| OVCAR-5 | | | |
| Ovarian ca. IGROV- | 2.3 | Stomach Pool | 0.0 |
| 1 | | | |
| Ovarian ca. | 0.3 | Bone Marrow Pool | 0.0 |
| OVCAR-8 | | | |
| Ovary | 1.8 | Fetal Heart | 0.0 |
| Breast ca. MCF-7 | 5.9 | Heart Pool | 0.0 |
| Breast ca. MDA- | 12.3 | Lymph Node Pool | 0.0 |
| MB-231 | | | |
| Breast ca. BT 549 | 0.0 | Fetal Skeletal Muscle | 0.0 |
| Breast ca. T47D | 30.1 | Skeletal Muscle Pool | 0.0 |
| Breast ca. MDA-N | 0.0 | Spleen Pool | 0.0 |
| Breast Pool | 0.0 | Thymus Pool | 0.0 |
| Trachea | 3.1 | CNS cancer (glio/astro) | 0.0 |
| | | U87-MG | |
| Lung | 0.0 | CNS cancer (glio/astro) | 0.0 |
| | | U-118-MG | |
| Fetal Lung | 4.2 | CNS cancer | 0.0 |
| | | (neuro; met) SK-N-AS | |
| Lung ca. NCI-N417 | 0.0 | CNS cancer (astro) SF- | 0.5 |
| | | 539 | |
| Lung ca. LX-1 | 1.9 | CNS cancer (astro) | 11.3 |
| | | SNB-75 | |
| Lung ca. NCI-H146 | 0.0 | CNS cancer (glio) | 2.6 |
| | | SNB-19 | |
| Lung ca. SHP-77 | 0.1 | CNS cancer (glio) SF- | 0.0 |
| | | 295 | |
| Lung ca. A549 | 8.1 | Brain (Amygdala) Pool | 0.0 |
| Lung ca. NCI-H526 | 0.0 | Brain (cerebellum) | 0.0 |
| Lung ca. NCI-H23 | 4.0 | Brain (fetal) | 0.0 |
| Lung ca. NCI-H460 | 0.0 | Brain (Hippocampus) | 0.0 |
| | | Pool | |
| Lung ca. HOP-62 | 0.5 | Cerebral Cortex Pool | 0.0 |
| Lung ca. NCI-H522 | 0.0 | Brain (Substantia nigra) | 0.0 |
| | | Pool | |
| Liver | 0.0 | Brain (Thalamus) Pool | 0.0 |
| Fetal Liver | 1.0 | Brain (whole) | 0.0 |
| Liver ca. HepG2 | 0.0 | Spinal Cord Pool | 0.0 |
| Kidney Pool | 0.0 | Adrenal Gland | 0.0 |
| Fetal Kidney | 0.0 | Pituitary gland Pool | 0.0 |
| Renal ca. 786-0 | 0.0 | Salivary Gland | 0.1 |
| Renal ca. A498 | 6.8 | Thyroid (female) | 3.5 |
| Renal ca. ACHN | 3.9 | Pancreatic ca. | 6.7 |
| | | CAPAN2 | |
| Renal ca. UO-31 | 0.0 | Pancreas Pool | 0.1 |

General_screening_panel_v1.4 Summary: Ag4202 Highest expression of the CG101068-01 gene is seen in a gastric cancer cell line (CT=26.5). Moderate levels of expression are seen in cell lines derived from pancreatic, brain, renal, lung, colon, breast and ovarian cancers. Thus, expression of this gene may be used to differentiate the gastric cancer cell line from other samples on this panel and as a marker of these cancers. This gene encodes a protein with homology to claudin, a family of proteins that are integral components of the tight junction. Members of this family have been shown to be upregulated in pancreatic cancer and colon cancer and in the former case proposed as novel targets for the treatment of this disease (Michl P. Gastroenterology September 2001;121(3):678-84; Miwa, N. Oncol Res 2001;12(11-12):469-76) Therefore, therapeutic modulation of the expression or function of this protein may be of use in the treatment of these cancers.

Claudin 11 has been shown to be a component of the CNS myelin and has been implicated in the regulation of growth and differentiation via signal transduction pathways.

Furthermore, evidence has been presented that shows that claudin 11 may be involved in the autoantigen that is responsible for the development of autoimmune demyelinating disease.(Bronstein J M. J Neurosci Res Mar. 15, 2000;59(6):706-11). Therefore, therapeutic modulation of the expression or function of this putative claudin may be of use in the treatment of demyelinating diseases such as multiple sclerosis and in restoring normal function to the CNS.

D. CG101231-01 and CG101231-02: Integral Membrane Protein

Expression of gene CG101231-01 and CG101231-02 was assessed using the primer-probe sets Ag4208 and Ag4997, described in Tables DA and DB. Results of the RTQ-PCR runs are shown in Tables DC and DD.

TABLE DA
Probe Name Ag4208

| | | | Start | SEQ ID |
|---|---|---|---|---|
| Primers | Sequences | Length | Position | No |
| Forward | 5′-cccactttgctcttacaaagac-3′ | 22 | 594 | 156 |
| Probe | TET-5′-actttcattgaaggcagatcatacca-3′-TAMRA | 26 | 622 | 157 |
| Reverse | 5′-ttttccattttcaccagattgt-3′ | 22 | 654 | 158 |

TABLE DB
Probe Name Ag4997

| | | | Start | SEQ ID |
|---|---|---|---|---|
| Primers | Sequences | Length | Position | No |
| Forward | 5′-ggagaaaattctttgggacaga-3′ | 22 | 1489 | 159 |
| Probe | TET-5′-caacaaacaatgtttgcaatcagaatca-3′-TAMRA | 28 | 1523 | 160 |
| Reverse | 5′-tgatgaatgtctcgaggctatt-3′ | 22 | 1554 | 161 |

TABLE DC
General_screening_panel_v1.4

| | Rel. Exp. (%) | | Rel. Exp. (%) |
|---|---|---|---|
| | Ag4997, Run | | Ag4997, Run |
| Tissue Name | 222456716 | Tissue Name | 222456716 |
| Adipose | 1.2 | Renal ca. TK-10 | 13.1 |
| Melanoma* | 26.4 | Bladder | 3.2 |
| Hs688(A).T | | | |
| Melanoma* | 30.6 | Gastric ca. (liver met.) | 36.3 |
| Hs688(B).T | | NCI-N87 | |
| Melanoma* M14 | 8.2 | Gastric ca. KATO III | 29.3 |
| Melanoma* | 9.0 | Colon ca. SW-948 | 4.0 |
| LOXIMVI | | | |
| Melanoma* SK- | 4.5 | Colon ca. SW480 | 33.4 |
| MEL-5 | | | |
| Squamous cell | 11.2 | Colon ca.* (SW480 | 13.5 |
| carcinoma SCC-4 | | met) SW620 | |
| Testis Pool | 2.8 | Colon ca. HT29 | 0.6 |
| Prostate ca.* (bone | 20.3 | Colon ca. HCT-116 | 10.8 |
| met) PC-3 | | | |
| Prostate Pool | 12.4 | Colon ca. CaCo-2 | 2.7 |
| Placenta | 0.3 | Colon cancer tissue | 5.8 |
| Uterus Pool | 1.4 | Colon ca. SW1116 | 8.9 |
| Ovarian ca. | 10.4 | Colon ca. Colo-205 | 5.9 |
| OVCAR-3 | | | |
| Ovarian ca. SK-OV- | 100.0 | Colon ca. SW-48 | 1.4 |
| 3 | | | |
| Ovarian ca. | 4.7 | Colon Pool | 5.6 |
| OVCAR-4 | | | |
| Ovarian ca. | 44.8 | Small Intestine Pool | 4.0 |
| OVCAR-5 | | | |
| Ovarian ca. IGROV- | 6.3 | Stomach Pool | 3.3 |
| 1 | | | |
| Ovarian ca. | 10.4 | Bone Marrow Pool | 2.0 |
| OVCAR-8 | | | |
| Ovary | 3.0 | Fetal Heart | 17.2 |
| Breast ca. MCF-7 | 11.0 | Heart Pool | 2.1 |
| Breast ca. MDA- | 22.4 | Lymph Node Pool | 6.9 |
| MB-231 | | | |
| Breast ca. BT 549 | 67.8 | Fetal Skeletal Muscle | 2.0 |
| Breast ca. T47D | 66.4 | Skeletal Muscle Pool | 0.5 |
| Breast ca. MDA-N | 6.9 | Spleen Pool | 1.3 |
| Breast Pool | 5.5 | Thymus Pool | 5.3 |
| Trachea | 5.3 | CNS cancer (glio/astro) | 21.3 |
| | | U87-MG | |
| Lung | 1.4 | CNS cancer (glio/astro) | 17.7 |
| | | U-118-MG | |
| Fetal Lung | 29.1 | CNS cancer | 12.3 |
| | | (neuro; met) SK-N-AS | |
| Lung ca. NCI-N417 | 2.4 | CNS cancer (astro) SF- | 18.0 |
| | | 539 | |
| Lung ca. LX-1 | 29.5 | CNS cancer (astro) | 49.7 |
| | | SNB-75 | |
| Lung ca. NCI-H146 | 12.0 | CNS cancer (glio) | 6.1 |
| | | SNB-19 | |
| Lung ca. SHP-77 | 17.8 | CNS cancer (glio) SF- | 20.6 |
| | | 295 | |
| Lung ca. A549 | 11.9 | Brain (Amygdala) Pool | 9.3 |
| Lung ca. NCI-H526 | 5.0 | Brain (cerebellum) | 6.9 |
| Lung ca. NCI-H23 | 11.3 | Brain (fetal) | 19.6 |
| Lung ca. NCI-H460 | 1.1 | Brain (Hippocampus) | 11.7 |
| | | Pool | |
| Lung ca. HOP-62 | 8.5 | Cerebral Cortex Pool | 14.9 |
| Lung ca. NCI-H522 | 19.6 | Brain (Substantia nigra) | 12.2 |
| | | Pool | |
| Liver | 0.0 | Brain (Thalamus) Pool | 17.2 |
| Fetal Liver | 21.5 | Brain (whole) | 10.4 |
| Liver ca. HepG2 | 0.0 | Spinal Cord Pool | 18.9 |
| Kidney Pool | 4.8 | Adrenal Gland | 0.7 |
| Fetal Kidney | 12.9 | Pituitary gland Pool | 3.3 |
| Renal ca. 786-0 | 11.6 | Salivary Gland | 1.0 |
| Renal ca. A498 | 5.4 | Thyroid (female) | 4.0 |
| Renal ca. ACHN | 4.9 | Pancreatic ca. | 52.9 |
| | | CAPAN2 | |
| Renal ca. UO-31 | 7.8 | Pancreas Pool | 5.3 |

TABLE DD
Panel 4.1D

| | Rel. Exp. (%) | | Rel. Exp. (%) |
|---|---|---|---|
| | Ag4997, Run | | Ag4997, Run |
| Tissue Name | 225428030 | Tissue Name | 225428030 |
| Secondary Th1 act | 2.7 | HUVEC IL-1beta | 3.2 |
| Secondary Th2 act | 3.8 | HUVEC IFN gamma | 4.0 |
| Secondary Tr1 act | 4.5 | HUVEC TNF alpha + IFN | 1.9 |
| | | gamma | |
| Secondary Th1 rest | 1.8 | HUVEC TNF alpha + IL4 | 3.0 |
| Secondary Th2 rest | 2.0 | HUVEC IL-11 | 11.9 |
| Secondary Tr1 rest | 1.4 | Lung Microvascular EC | 8.8 |
| | | none | |
| Primary Th1 act | 1.9 | Lung Microvascular EC | 3.9 |
| | | TNFalpha + IL-1 beta | |
| Primary Th2 act | 3.9 | Microvascular Dermal EC | 7.5 |
| | | none | |
| Primary Tr1 act | 3.5 | Microsvasular Dermal EC | 3.1 |
| | | TNFalpha + IL-1beta | |
| Primary Th1 rest | 1.7 | Bronchial epithelium | 38.7 |
| | | TNFalpha + IL1beta | |
| Primary Th2 rest | 3.9 | Small airway epithelium | 14.8 |
| | | none | |
| Primary Tr1 rest | 8.5 | Small airway epithelium | 24.0 |
| | | TNFalpha + IL-1beta | |
| CD45RA CD4 | 7.9 | Coronery artery SMC rest | 13.3 |
| lymphocyte act | | | |
| CD45RO CD4 | 1.6 | Coronery artery SMC | 12.9 |
| lymphocyte act | | TNFalpha + IL-1beta | |
| CD8 lymphocyte act | 1.4 | Astrocytes rest | 10.4 |
| Secondary CD8 | 1.7 | Astrocytes TNFalpha + | 12.0 |
| lymphocyte rest | | IL-1beta | |
| Secondary CD8 | 0.4 | KU-812 (Basophil) rest | 78.5 |
| lymphocyte act | | | |
| CD4 lymphocyte none | 0.2 | KU-812 (Basophil) | 100.0 |
| | | PMA/ionomycin | |
| 2ry Th1/Th2/Tr1_anti- | 2.0 | CCD1106 (Keratinocytes) | 31.2 |
| CD95 CH11 | | none | |
| LAK cells rest | 1.6 | CCD1106 (Keratinocytes) | 17.0 |
| | | TNFalpha + IL-1beta | |
| LAK cells IL-2 | 3.0 | Liver cirrhosis | 1.9 |
| LAK cells IL-2 + IL-12 | 1.0 | NCI-H292 none | 9.5 |
| LAK cells IL-2 + IFN | 1.6 | NCI-H292 IL-4 | 12.1 |
| gamma | | | |
| LAK cells IL-2 + IL-18 | 1.3 | NCI-H292 IL-9 | 14.8 |
| LAK cells | 1.3 | NCI-H292 IL-13 | 11.7 |
| PMA/ionomycin | | | |
| NK Cells IL-2 rest | 3.2 | NCI-H292 IFN gamma | 8.5 |
| Two Way MLR 3 day | 0.5 | HPAEC none | 4.9 |
| Two Way MLR 5 day | 2.2 | HPAEC TNF alpha + IL-1 | 3.8 |
| | | beta | |
| Two Way MLR 7 day | 0.4 | Lung fibroblast none | 13.4 |
| PBMC rest | 1.8 | Lung fibroblast TNF alpha + | 17.2 |
| | | IL-1 beta | |
| PBMC PWM | 0.5 | Lung fibroblast IL-4 | 11.3 |
| PBMC PHA-L | 1.4 | Lung fibroblast IL-9 | 22.7 |
| Ramos (B cell) none | 0.0 | Lung fibroblast IL-13 | 8.9 |
| Ramos (B cell) | 0.0 | Lung fibroblast IFN | 8.7 |
| ionomycin | | gamma | |
| B lymphocytes PWM | 1.9 | Dermal fibroblast | 24.8 |
| | | CCD1070 rest | |
| B lymphocytes CD40L | 3.0 | Dermal fibroblast | 13.8 |
| and IL-4 | | CCD1070 TNF alpha | |
| EOL-1 dbcAMP | 0.1 | Dermal fibroblast | 8.2 |
| | | CCD1070 IL-1 beta | |
| EOL-1 dbcAMP | 0.0 | Dermal fibroblast IFN | 7.9 |
| PMA/ionomycin | | gamma | |
| Dendritic cells none | 3.1 | Dermal fibroblast IL-4 | 15.7 |
| Dendritic cells LPS | 5.2 | Dermal Fibroblasts rest | 12.9 |
| Dendritic cells anti- | 3.7 | Neutrophils TNFa + LPS | 3.3 |
| CD40 | | | |
| Monocytes rest | 3.8 | Neutrophils rest | 20.2 |
| Monocytes LPS | 1.6 | Colon | 2.9 |
| Macrophages rest | 1.3 | Lung | 5.7 |
| Macrophages LPS | 0.1 | Thymus | 6.4 |
| HUVEC none | 3.6 | Kidney | 22.4 |
| HUVEC starved | 5.6 | | |

General_screening_panel_v1.4 Summary: Ag4997 Highest expression of the CG101231-01 gene is detected in an ovarian cancer SK-OV-3 cell line (CT=27). In addition, expression of this gene is also seen in cluster of cancer cell lines including pancreatic, CNS, colon, gastric, renal, lung, breast, ovarian, prostate, squamous cell carcinoma, and melanoma cancer cell lines. Overall, expression of this gene appears to be higher in samples derived from cancer cell lines than in normal tissues. Thus, expression of this gene could be used as a marker to detect the presence of cancer. Furthermore, therapeutic modulation of the expression or function of this gene product may be useful in the treatment of these cancers.

Among tissues with metabolic or endocrine function, this gene is expressed at low to moderate levels in pancreas, adipose, adrenal gland, thyroid, pituitary gland, skeletal muscle, heart, liver and the gastrointestinal tract. Therefore, therapeutic modulation of the activity of this gene may prove useful in the treatment of endocrine/metabolically related diseases, such as obesity and diabetes.

Interestingly, this gene is expressed at much higher levels in fetal (CTs=29) when compared to adult lung and liver(CTs=33-38). This observation suggests that expression of this gene can be used to distinguish fetal from adult lung and liver. In addition, the relative overexpression of this gene in fetal tissue suggests that the protein product may enhance growth or development of liver and lung in the fetus and thus may also act in a regenerative capacity in the adult. Therefore, therapeutic modulation of the protein encoded by this gene could be useful in treatment of liver and lung related diseases.

In addition, this gene is expressed at moderate levels in all regions of the central nervous system examined, including amygdala, hippocampus, substantia nigra, thalamus, cerebellum, cerebral cortex, and spinal cord. Therefore, this gene may play an important role in central nervous system and therapeutic modulation of this gene product may be useful in the treatment of neurological disorders such as Alzheimer's disease, Parkinson's disease, epilepsy, multiple sclerosis, schizophrenia and depression.

Panel 4.1D Summary: Ag4997 Highest expression of the CG101231-01 gene is detected in PMA/ionomycin treated basophils (CT=29.4). This gene is expressed at low to moderate levels in a wide range of cell types of significance in the immune response in health and disease. These cells include members of the T-cell, lymphocytes, endothelial cell, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. This pattern is in agreement with the expression profile in General_screening_panel_v1.4 and also suggests a role for the gene product in cell survival and proliferation. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

E. CG101362-01: Prion Protein

Expression of gene CG101362-01 was assessed using the primer-probe set Ag6902, described in Table EA. Results of the RTQ-PCR runs are shown in Table EB.

TABLE EA
Probe Name Ag6902

| | | | Start | SEQ ID |
|---|---|---|---|---|
| Primers | Sequences | Length | Position | No |
| Forward | 5′-gaacactgggagcagatgtg-3′ | 20 | 173 | 162 |
| Probe | TET-5′-aggaccatgctcgatcctctctggtaata-3′-TAMRA | 29 | 224 | 163 |
| Reverse | 5′-aggaggatcacaggtggaga-3′ | 20 | 260 | 164 |

TABLE EB
General_screening_panel_v1.6

| | Rel. Exp. (%) | | Rel. Exp. (%) |
|---|---|---|---|
| | Ag6902, Run | | Ag6902, Run |
| Tissue Name | 278388399 | Tissue Name | 278388399 |
| Adipose | 3.0 | Renal ca. TK-10 | 9.0 |
| Melanoma* | 23.5 | Bladder | 3.8 |
| Hs688(A).T | | | |
| Melanoma* | 21.0 | Gastric ca. (liver met.) | 21.2 |
| Hs688(B).T | | NCI-N87 | |
| Melanoma* M14 | 50.3 | Gastric ca. KATO III | 15.9 |
| Melanoma* | 38.7 | Colon ca. SW-948 | 2.6 |
| LOXIMVI | | | |
| Melanoma* SK- | 14.3 | Colon ca. SW480 | 24.5 |
| MEL-5 | | | |
| Squamous cell | 24.0 | Colon ca.* (SW480 | 16.8 |
| carcinoma SCC-4 | | met) SW620 | |
| Testis Pool | 6.5 | Colon ca. HT29 | 5.3 |
| Prostate ca.* (bone | 100.0 | Colon ca. HCT-116 | 12.2 |
| met) PC-3 | | | |
| Prostate Pool | 6.4 | Colon ca. CaCo-2 | 4.1 |
| Placenta | 1.5 | Colon cancer tissue | 6.6 |
| Uterus Pool | 4.1 | Colon ca. SW1116 | 1.5 |
| Ovarian ca. | 2.5 | Colon ca. Colo-205 | 2.3 |
| OVCAR-3 | | | |
| Ovarian ca. SK-OV- | 6.1 | Colon ca. SW-48 | 0.7 |
| 3 | | | |
| Ovarian ca. | 3.3 | Colon Pool | 9.6 |
| OVCAR-4 | | | |
| Ovarian ca. | 16.3 | Small Intestine Pool | 8.5 |
| OVCAR-5 | | | |
| Ovarian ca. IGROV- | 21.9 | Stomach Pool | 4.0 |
| 1 | | | |
| Ovarian ca. | 16.8 | Bone Marrow Pool | 4.9 |
| OVCAR-8 | | | |
| Ovary | 6.0 | Fetal Heart | 8.2 |
| Breast ca. MCF-7 | 2.2 | Heart Pool | 5.6 |
| Breast ca. MDA- | 32.1 | Lymph Node Pool | 11.3 |
| MB-231 | | | |
| Breast ca. BT 549 | 41.2 | Fetal Skeletal Muscle | 4.0 |
| Breast ca. T47D | 1.7 | Skeletal Muscle Pool | 0.7 |
| Breast ca. MDA-N | 7.1 | Spleen Pool | 1.6 |
| Breast Pool | 12.1 | Thymus Pool | 3.7 |
| Trachea | 5.4 | CNS cancer (glio/astro) | 31.4 |
| | | U87-MG | |
| Lung | 2.1 | CNS cancer (glio/astro) | 30.4 |
| | | U-118-MG | |
| Fetal Lung | 13.4 | CNS cancer | 2.7 |
| | | (neuro; met) SK-N-AS | |
| Lung ca. NCI-N417 | 1.6 | CNS cancer (astro) SF- | 7.5 |
| | | 539 | |
| Lung ca. LX-1 | 20.3 | CNS cancer (astro) | 32.8 |
| | | SNB-75 | |
| Lung ca. NCI-H146 | 1.0 | CNS cancer (glio) | 20.2 |
| | | SNB-19 | |
| Lung ca. SHP-77 | 1.7 | CNS cancer (glio) SF- | 66.4 |
| | | 295 | |
| Lung ca. A549 | 17.6 | Brain (Amygdala) Pool | 18.6 |
| Lung ca. NCI-H526 | 1.4 | Brain (cerebellum) | 67.8 |
| Lung ca. NCI-H23 | 23.8 | Brain (fetal) | 10.9 |
| Lung ca. NCI-H460 | 18.7 | Brain (Hippocampus) | 21.9 |
| | | Pool | |
| Lung ca. HOP-62 | 28.5 | Cerebral Cortex Pool | 33.0 |
| Lung ca. NCI-H522 | 4.0 | Brain (Substantia nigra) | 17.7 |
| | | Pool | |
| Liver | 0.5 | Brain (Thalamus) Pool | 30.4 |
| Fetal Liver | 5.5 | Brain (whole) | 29.3 |
| Liver ca. HepG2 | 8.0 | Spinal Cord Pool | 13.2 |
| Kidney Pool | 14.0 | Adrenal Gland | 5.4 |
| Fetal Kidney | 2.4 | Pituitary gland Pool | 3.0 |
| Renal ca. 786-0 | 8.0 | Salivary Gland | 2.2 |
| Renal ca. A498 | 3.8 | Thyroid (female) | 3.5 |
| Renal ca. ACHN | 10.6 | Pancreatic ca. | 13.0 |
| | | CAPAN2 | |
| Renal ca. UO-31 | 26.1 | Pancreas Pool | 3.7 |

General_screening_panel_v1.6 Summary: Ag6902 Highest expression of the CG101362-01 gene is detected in prostate cancer cell line (CT=29.5). Moderate to low levels of expression of this gene is also seen in cluster of cancer cell lines derived from gastric, colon, lung, renal, breast, ovarian, prostate, squamous cell carcinoma, melanoma and brain cancers. Thus, expression of this gene could be used as a marker to detect the presence of these cancers. Furthermore, therapeutic modulation of the expression or function of this gene may be effective in the treatment of gastric, colon, lung, renal, breast, ovarian, prostate, squamous cell carcinoma, melanoma and brain cancers.

Among tissues with metabolic or endocrine function, this gene is expressed at moderate to low levels in pancreas, adipose, adrenal gland, thyroid, pituitary gland, fetal skeletal muscle, heart, fetal liver and the gastrointestinal tract. Therefore, therapeutic modulation of the activity of this gene may prove useful in the treatment of endocrine/metabolically related diseases, such as obesity and diabetes.

Interestingly, this gene is expressed at much higher levels in fetal (CT=33.7) when compared to adult liver (CT=37). This observation suggests that expression of this gene can be used to distinguish fetal from adult liver. In addition, the relative overexpression of this gene in fetal liver suggests that the protein product may enhance liver growth or development in the fetus and thus may also act in a regenerative capacity in the adult. Therefore, therapeutic modulation of the protein encoded by this gene could be useful in treatment of liver related diseases.

In addition, this gene is expressed at moderate levels in all regions of the central nervous system examined, including amygdala, hippocampus, substantia nigra, thalamus, cerebellum, cerebral cortex, and spinal cord. Therefore, therapeutic modulation of this gene product may be useful in the treatment of central nervous system disorders such as Alzheimer's disease, Parkinson's disease, epilepsy, multiple sclerosis, schizophrenia and depression.

F. CG101458-01: von Willebrand Domain Protein

Expression of gene CG101458-01 was assessed using the primer-probe set Ag4220, described in Table FA. Results of the RTQ-PCR runs are shown in Tables FB, FC, FD and FE.

TABLE FA
Probe Name Ag4220

| | | | Start | SEQ ID |
|---|---|---|---|---|
| Primers | Sequences | Length | position | No |
| Forward | 5′-ccaacaagcacacctttgac-3′ | 20 | 777 | 165 |
| Probe | TET-5′-ctgtggagatcctcatccaccccag-3′-TAMRA | 25 | 801 | 166 |
| Reverse | 5′-ctatcaggacatggggcata-3′ | 20 | 835 | 167 |

TABLE FB
CNS_neurodegeneration_v1.0

| | Rel. Exp.(%) | | Rel. Exp.(%) |
|---|---|---|---|
| | Ag4220, Run | | Ag4220, Run |
| Tissue Name | 215620528 | Tissue Name | 215620528 |
| AD 1 Hippo | 12.3 | Control (Path) 3 | 0.0 |
| | | Temporal Ctx | |
| AD 2 Hippo | 32.8 | Control (Path) 4 | 7.5 |
| | | Temporal Ctx | |
| AD 3 Hippo | 18.6 | AD 1 Occipital Ctx | 3.1 |
| AD 4 Hippo | 22.2 | AD 2 Occipital Ctx | 0.0 |
| | | (Missing) | |
| AD 5 Hippo | 7.1 | AD 3 Occipital Ctx | 3.6 |
| AD 6 Hippo | 11.6 | AD 4 Occipital Ctx | 11.5 |
| Control 2 Hippo | 19.6 | AD 5 Occipital Ctx | 8.7 |
| Control 4 Hippo | 43.8 | AD 5 Occipital Ctx | 2.3 |
| Control (Path) 3 | 98.6 | Control 1 Occipital | 1.8 |
| Hippo | | Ctx | |
| AD 1 Temporal Ctx | 4.9 | Control 2 Occipital | 5.5 |
| | | Ctx | |
| AD 2 Temporal Ctx | 12.8 | Control 3 Occipital | 0.7 |
| | | Ctx | |
| AD 3 Temporal Ctx | 2.8 | Control 4 Occipital | 12.2 |
| | | Ctx | |
| AD 4 Temporal Ctx | 12.9 | Control (Path) 1 | 30.8 |
| | | Occipital Ctx | |
| AD 5 Inf Temporal | 7.2 | Control (Path) 2 | 2.8 |
| Ctx | | Occipital Ctx | |
| AD 5 Sup Temporal | 100.0 | Control (Path) 3 | 1.5 |
| Ctx | | Occipital Ctx | |
| AD 6 Inf Temporal | 8.1 | Control (Path) 4 | 2.2 |
| Ctx | | Occipital Ctx | |
| AD 6 Sup Temporal | 15.1 | Control 1 Parietal Ctx | 2.6 |
| Ctx | | | |
| Control 1 Temporal | 3.6 | Control 2 Parietal Ctx | 17.0 |
| Ctx | | | |
| Control 2 Temporal | 2.7 | Control 3 Parietal Ctx | 1.4 |
| Ctx | | | |
| Control 3 Temporal | 6.4 | Control (Path) 1 | 14.2 |
| Ctx | | Parietal Ctx | |
| Control 3 Temporal | 5.8 | Control (Path) 2 | 15.6 |
| Ctx | | Parietal Ctx | |
| Control (Path) 1 | 11.0 | Control (Path) 3 | 0.0 |
| Temporal Ctx | | Parietal Ctx | |
| Control (Path) 2 | 5.0 | Control (Path) 4 | 17.8 |
| Temporal Ctx | | Parietal Ctx | |

TABLE FC
General_screening_panel_v1.4

| | Rel. Exp. (%) | | Rel. Exp. (%) |
|---|---|---|---|
| | Ag4220, Run | | Ag4220, Run |
| Tissue Name | 215620528 | Tissue Name | 215620528 |
| Adipose | 0.4 | Renal ca. TK-10 | 0.0 |
| Melanoma* | 0.0 | Bladder | 1.5 |
| Hs688(A).T | | | |
| Melanoma* | 0.0 | Gastric ca. (liver met.) | 0.0 |
| Hs688(B).T | | NCI-N87 | |
| Melanoma* M14 | 0.0 | Gastric ca. KATO III | 0.0 |
| Melanoma* | 0.0 | Colon ca. SW-948 | 0.0 |
| LOXIMVI | | | |
| Melanoma* SK- | 0.0 | Colon ca. SW480 | 0.0 |
| MEL-5 | | | |
| Squamous cell | 0.0 | Colon ca.* (SW480 | 0.0 |
| carcinoma SCC-4 | | met) SW620 | |
| Testis Pool | 35.1 | Colon ca. HT29 | 0.0 |
| Prostate ca.* (bone | 0.0 | Colon ca. HCT-116 | 0.0 |
| met) PC-3 | | | |
| Prostate Pool | 0.1 | Colon ca. CaCo-2 | 0.0 |
| Placenta | 0.0 | Colon cancer tissue | 13.1 |
| Uterus Pool | 0.0 | Colon ca. SW1116 | 0.0 |
| Ovarian ca. | 0.0 | Colon ca. Colo-205 | 0.0 |
| OVCAR-3 | | | |
| Ovarian ca. SK-OV- | 0.0 | Colon ca. SW-48 | 2.7 |
| 3 | | | |
| Ovarian ca. | 0.5 | Colon Pool | 0.0 |
| OVCAR-4 | | | |
| Ovarian ca. | 0.6 | Small Intestine Pool | 1.9 |
| OVCAR-5 | | | |
| Ovarian ca. IGROV- | 1.2 | Stomach Pool | 0.1 |
| 1 | | | |
| Ovarian ca. | 0.0 | Bone Marrow Pool | 0.4 |
| OVCAR-8 | | | |
| Ovary | 0.0 | Fetal Heart | 0.0 |
| Breast ca. MCF-7 | 0.0 | Heart Pool | 0.2 |
| Breast ca. MDA- | 0.0 | Lymph Node Pool | 0.0 |
| MB-231 | | | |
| Breast ca. BT 549 | 0.0 | Fetal Skeletal Muscle | 0.6 |
| Breast ca. T47D | 0.7 | Skeletal Muscle Pool | 0.9 |
| Breast ca. MDA-N | 0.0 | Spleen Pool | 0.5 |
| Breast Pool | 1.0 | Thymus Pool | 0.5 |
| Trachea | 0.4 | CNS cancer (glio/astro) | 0.0 |
| | | U87-MG | |
| Lung | 0.0 | CNS cancer (glio/astro) | 0.0 |
| | | U-118-MG | |
| Fetal Lung | 3.7 | CNS cancer | 0.0 |
| | | (neuro; met) SK-N-AS | |
| Lung ca. NCI-N417 | 0.5 | CNS cancer (astro) SF- | 0.0 |
| | | 539 | |
| Lung ca. LX-1 | 0.3 | CNS cancer (astro) | 0.0 |
| | | SNB-75 | |
| Lung ca. NCI-H146 | 0.0 | CNS cancer (glio) | 2.7 |
| | | SNB-19 | |
| Lung ca. SHP-77 | 0.0 | CNS cancer (glio) SF- | 0.7 |
| | | 295 | |
| Lung ca. A549 | 0.3 | Brain (Amygdala) Pool | 1.3 |
| Lung ca. NCI-H526 | 0.0 | Brain (cerebellum) | 0.9 |
| Lung ca. NCI-H23 | 0.0 | Brain (fetal) | 6.8 |
| Lung ca. NCI-H460 | 0.0 | Brain (Hippocampus) | 8.0 |
| | | Pool | |
| Lung ca. HOP-62 | 0.0 | Cerebral Cortex Pool | 2.9 |
| Lung ca. NCI-H522 | 0.0 | Brain (Substantia nigra) | 6.7 |
| | | Pool | |
| Liver | 0.0 | Brain (Thalamus) Pool | 8.8 |
| Fetal Liver | 0.0 | Brain (whole) | 5.2 |
| Liver ca. HepG2 | 0.0 | Spinal Cord Pool | 8.9 |
| Kidney Pool | 0.0 | Adrenal Gland | 7.7 |
| Fetal Kidney | 21.9 | Pituitary gland Pool | 100.0 |
| Renal ca. 786-0 | 0.0 | Salivary Gland | 17.6 |
| Renal ca. A498 | 0.0 | Thyroid (female) | 8.6 |
| Renal ca. ACHN | 0.0 | Pancreatic ca. | 0.0 |
| | | CAPAN2 | |
| Renal ca. UO-31 | 2.4 | Pancreas Pool | 5.1 |

TABLE FD
Panel 4.1D

| | Rel. Exp. (%) | | Rel. Exp. (%) |
|---|---|---|---|
| | Ag4220, Run | | Ag4220, Run |
| Tissue Name | 174926565 | Tissue Name | 174926565 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC | 0.0 |
| | | none | |
| Primary Th1 act | 0.0 | Lung Microvascular EC | 0.0 |
| | | TNFalpha + IL-1 beta | |
| Primary Th2 act | 0.0 | Microvascular Dermal EC | 0.0 |
| | | none | |
| Primary Tr1 act | 0.0 | Microsvasular Dermal EC | 0.0 |
| | | TNFalpha + IL-1beta | |
| Primary Th1 rest | 0.0 | Bronchial epithelium | 0.0 |
| | | TNFalpha + IL1beta | |
| Primary Th2 rest | 0.0 | Small airway epithelium | 0.0 |
| | | none | |
| Primary Tr1 rest | 0.0 | Small airway epithelium | 0.0 |
| | | TNFalpha + IL-1beta | |
| CD45RA CD4 | 1.5 | Coronery artery SMC rest | 0.0 |
| lymphocyte act | | | |
| CD45RO CD4 | 2.4 | Coronery artery SMC | 0.0 |
| lymphocyte act | | TNFalpha + IL-1beta | |
| CD8 lymphocyte act | 2.5 | Astrocytes rest | 0.0 |
| Secondary CD8 | 0.0 | Astrocytes TNFalpha + | 0.0 |
| lymphocyte rest | | IL-1beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CCD1106 (Keratinocytes) | 0.0 |
| CD95 CH11 | | none | |
| LAK cells rest | 0.0 | CCD1106 (Keratinocytes) | 0.0 |
| | | TNFalpha + IL-1beta | |
| LAK cells IL-2 | 0.0 | Liver cirrhosis | 0.0 |
| LAK cells IL-2 + IL-12 | 0.0 | NCI-H292 none | 0.0 |
| LAK cells IL-2 + IFN | 0.0 | NCI-H292 IL-4 | 0.0 |
| gamma | | | |
| LAK cells IL-2 + IL-18 | 1.5 | NCI-H292 IL-9 | 0.0 |
| LAK cells | 0.0 | NCI-H292 IL-13 | 0.0 |
| PMA/ionomycin | | | |
| NK Cells IL-2 rest | 0.0 | NCI-H292 IFN gamma | 0.0 |
| Two Way MLR 3 day | 2.5 | HPAEC none | 0.0 |
| Two Way MLR 5 day | 0.0 | HPAEC TNF alpha + IL-1 | 0.0 |
| | | beta | |
| Two Way MLR 7 day | 0.0 | Lung fibroblast none | 0.0 |
| PBMC rest | 0.0 | Lung fibroblast TNF alpha + | 0.0 |
| | | IL-1 beta | |
| PBMC PWM | 0.0 | Lung fibroblast IL-4 | 0.0 |
| PBMC PHA-L | 2.8 | Lung fibroblast IL-9 | 0.0 |
| Ramos (B cell) none | 0.0 | Lung fibroblast IL-13 | 0.0 |
| Ramos (B cell) | 0.0 | Lung fibroblast IFN | 0.0 |
| ionomycin | | gamma | |
| B lymphocytes PWM | 0.0 | Dermal fibroblast | 0.0 |
| | | CCD1070 rest | |
| B lymphocytes CD40L | 0.0 | Dermal fibroblast | 0.0 |
| and IL-4 | | CCD1070 TNF alpha | |
| EOL-1 dbcAMP | 0.0 | Dermal fibroblast | 0.0 |
| | | CCD1070 IL-1 beta | |
| EOL-1 dbcAMP | 0.0 | Dermal fibroblast IFN | 0.0 |
| PMA/ionomycin | | gamma | |
| Dendritic cells none | 0.0 | Dermal fibroblast IL-4 | 5.4 |
| Dendritic cells LPS | 0.0 | Dermal Fibroblasts rest | 2.8 |
| Dendritic cells anti- | 0.0 | Neutrophils TNFa + LPS | 0.0 |
| CD40 | | | |
| Monocytes rest | 0.0 | Neutrophils rest | 0.0 |
| Monocytes LPS | 0.0 | Colon | 0.0 |
| Macrophages rest | 0.0 | Lung | 2.9 |
| Macrophages LPS | 0.0 | Thymus | 11.0 |
| HUVEC none | 0.0 | Kidney | 100.0 |
| HUVEC starved | 0.0 | | |

TABLE FE
general oncology screening panel_v_2.4

| | Rel. Exp. (%) | | Rel. Exp. (%) |
|---|---|---|---|
| | Ag4220, Run | | Ag4220, Run |
| Tissue Name | 268624960 | Tissue Name | 268624960 |
| Colon cancer 1 | 0.8 | Bladder cancer | 0.0 |
| | | NAT 2 | |
| Colon cancer | 0.0 | Bladder cancer | 0.0 |
| NAT 1 | | NAT 3 | |
| Colon cancer 2 | 0.0 | Bladder cancer | 0.0 |
| | | NAT 4 | |
| Colon cancer | 3.1 | Adenocarcinoma of | 0.6 |
| NAT 2 | | the prostate 1 | |
| Colon cancer 3 | 1.8 | Adenocarcinoma of | 0.0 |
| | | the prostate 2 | |
| Colon cancer | 5.3 | Adenocarcinoma of | 3.0 |
| NAT 3 | | the prostate 3 | |
| Colon malignant | 0.0 | Adenocarcinoma of | 3.6 |
| cancer 4 | | the prostate 4 | |
| Colon normal | 0.0 | Prostate cancer | 0.0 |
| adjacent tissue | | NAT 5 | |
| 4 | | | |
| Lung cancer 1 | 0.0 | Adenocarcinoma of | 0.0 |
| | | the prostate 6 | |
| Lung NAT 1 | 0.7 | Adenocarcinoma of | 1.9 |
| | | the prostate 7 | |
| Lung cancer 2 | 16.5 | Adenocarcinoma of | 0.0 |
| | | the prostate 8 | |
| Lung NAT 2 | 0.7 | Adenocarcinoma of | 3.3 |
| | | the prostate 9 | |
| Squamous cell | 0.0 | Prostate cancer | 0.7 |
| carcinoma 3 | | NAT 10 | |
| Lung NAT 3 | 0.0 | Kidney cancer 1 | 0.8 |
| metastatic | 27.4 | Kidney NAT 1 | 89.5 |
| melanoma 1 | | | |
| Melanoma 2 | 1.0 | Kidney cancer 2 | 13.9 |
| Melanoma 3 | 0.8 | Kidney NAT 2 | 100.0 |
| metastatic | 0.9 | Kidney cancer 3 | 3.0 |
| melanoma 4 | | | |
| metastatic | 0.0 | Kidney NAT 3 | 92.7 |
| melanoma 5 | | | |
| Bladder cancer | 0.0 | Kidney cancer 4 | 0.0 |
| 1 | | | |
| Bladder cancer | 0.0 | Kidney NAT 4 | 17.2 |
| NAT 1 | | | |
| Bladder cancer | 25.9 | | |
| 2 | | | |

CNS_neurodegeneration_v1.0 Summary: Ag4220 This panel confirms the expression of the CG101458-01 gene at low levels in the brains of an independent group of individuals. However, no differential expression of this gene was detected between Alzheimer's diseased postmortem brains and those of non-demented controls in this experiment. Please see Panel 1.4 for a discussion of the potential use of this gene in treatment of central nervous system disorders.

General_screening_panel_v1.4 Summary: Ag4220 Highest expression of the CG101458-01 gene is detected in pituitary gland (CT=27.9). Furthermore, moderate to low levels of expression of this gene is also seen in other tissues with metabolic or endocrine functions including pancrease, adrenal gland, thyroid, skeletal muscle, and small intestine. Therefore, therapeutic modulation of the activity of this gene may prove useful in the treatment of endocrine/metabolically related diseases, such as obesity and diabetes.

In addition, this gene is expressed at moderate to low levels in all regions of the central nervous system examined, including amygdala, hippocampus, substantia nigra, thalamus, cerebellum, cerebral cortex, and spinal cord. Therefore, this gene may play a role in central nervous system disorders such as Alzheimer's disease, Parkinson's disease, epilepsy, multiple sclerosis, schizophrenia and depression.

Moderate expression of this gene is also seen in testis. Therefore, therapeutic modulation of this gene product may be useful in the treatment of testis related diseases such as fertility and hypogonadism.

Significant expression of this gene is seen in fetal kidney and lung. Interestingly, this gene is expressed at much higher levels in fetal (CTs=30-32) when compared to adult kidney and lung(CTs=40). This observation suggests that expression of this gene can be used to distinguish fetal from adult kidney and lung. In addition, the relative overexpression of this gene in fetal tissue suggests that the protein product may enhance growth or development of kidney and lung in the fetus and thus may also act in a regenerative capacity in the adult. Therefore, therapeutic modulation of the protein encoded by this gene could be useful in treatment of kidney and lung related diseases.

Panel 4.1D Summary: Ag4220 Significant expression of the CG101458-01 gene is detected exclusively in kidney (CT=32.3). Therefore, expression of this gene may be used to distinguish kidney sample from other samples in this panel. In addition, therapeutic modulation of this gene product may be beneficial in the treatment of autoimmune and inflammatory diseases that affect kidney, including lupus and golomerulonephritis.

general oncology screening panel_v_{—}2.4 Summary: Ag4220 Highest expression of the CG101458-01 gene is detected in control kidney samples (CTs=31). Interestingly, expression of this gene is higher in control samples as compared to kidney cancer samples. Therefore, expression of this gene may be used to distinguish between cancer and normal kidney samples. In addition, therapeutic modulation of this gene product that stimulates the function or expression of the this gene product may be beneficial in the treatment of kidney cancer.

In addition, significant expression of this gene is also seen in a bladder cancer, a lung cancer and a metastatic melanoma samples. Expression of this gene is higher in these cancer samples as compared to the adjacent control samples. Therefore, expression of this gene may be used as diagnostic marker for detection of these cancers and therapeutic modulation of this gene product may be beneficial in the treatment of these cancers.

G. CG101475-01: Novel Plasma Membrane Protein Containing Lectin C-type Domain

Expression of gene CG101475-01 was assessed using the primer-probe set Ag4214, described in Table GA. Results of the RTQ-PCR runs are shown in Tables GB, GC and GD.

TABLE GA
Probe Name Ag4214

| | | | Start | SEQ ID |
|---|---|---|---|---|
| Primers | Sequences | Length | Position | No |
| Forward | 5′-gggatgatgtgtttgcagatat-3′ | 22 | 195 | 168 |
| Probe | TET-5′-cagagaaattgagtcaacttcagaaaacca-5′-TAMRA | 30 | 238 | 169 |
| Reverse | 5′-agttatcctgctgctgttgga-3′ | 21 | 268 | 170 |

TABLE GB
A1_comprehensive panel_v1.0

| | Rel. Exp. (%) | | Rel. Exp. (%) |
|---|---|---|---|
| | Ag4214, Run | | Ag4214, Run |
| Tissue Name | 248080020 | Tissue Name | 248080020 |
| 110967 COPD-F | 1.3 | 112427 Match Control | 3.9 |
| | | Psoriasis-F | |
| 110980 COPD-F | 0.0 | 112418 Psoriasis-M | 2.7 |
| 110968 COPD-M | 3.1 | 112723 Match Control | 8.8 |
| | | Psoriasis-M | |
| 110977 COPD-M | 0.3 | 112419 Psoriasis-M | 1.5 |
| 110989 Emphysema-F | 20.3 | 112424 Match Control | 2.3 |
| | | Psoriasis-M | |
| 110992 Emphysema-F | 1.5 | 112420 Psoriasis-M | 3.9 |
| 110993 Emphysema-F | 7.0 | 112425 Match Control | 2.7 |
| | | Psoriasis-M | |
| 110994 Emphysema-F | 3.3 | 104689 (MF) OA Bone- | 11.0 |
| | | Backus | |
| 110995 Emphysema-F | 3.1 | 104690 (MF) Adj “Normal” | 6.7 |
| | | Bone-Backus | |
| 110996 Emphysema-F | 0.0 | 104691 (MF) OA | 1.3 |
| | | Synovium-Backus | |
| 110997 Asthma-M | 2.6 | 104692 (BA) OA Cartilage- | 0.0 |
| | | Backus | |
| 111001 Asthma-F | 21.6 | 104694 (BA) OA Bone- | 1.6 |
| | | Backus | |
| 111002 Asthma-F | 11.5 | 104695 (BA) Adj “Normal” | 3.8 |
| | | Bone-Backus | |
| 111003 Atopic Asthma-F | 49.3 | 104696 (BA) OA | 9.8 |
| | | Synovium-Backus | |
| 111004 Atopic Asthma-F | 19.6 | 104700 (SS) OA Bone- | 1.4 |
| | | Backus | |
| 111005 Atopic Asthma-F | 24.5 | 104701 (SS) Adj “Normal” | 1.6 |
| | | Bone-Backus | |
| 111006 Atopic Asthma-F | 7.4 | 104702 (SS) OA | 35.4 |
| | | Synovium-Backus | |
| 111417 Allergy-M | 11.6 | 117093 OA Cartilage Rep7 | 2.6 |
| 112347 Allergy-M | 0.0 | 112672 OA Bone5 | 1.4 |
| 112349 Normal Lung-F | 0.0 | 112673 OA Synovium5 | 1.2 |
| 112357 Normal Lung-F | 10.6 | 112674 OA Synovial Fluid | 0.4 |
| | | cells5 | |
| 112354 Normal Lung-M | 0.8 | 117100 OA Cartilage | 0.6 |
| | | Rep14 | |
| 112374 Crohns-F | 2.9 | 112756 OA Bone9 | 8.9 |
| 112389 Match Control | 0.5 | 112757 OA Synovium9 | 0.0 |
| Crohns-F | | | |
| 112375 Crohns-F | 6.4 | 112758 OA Synovial Fluid | 6.0 |
| | | Cells9 | |
| 112732 Match Control | 0.0 | 117125 RA Cartilage Rep2 | 16.4 |
| Crohns-F | | | |
| 112725 Crohns-M | 0.0 | 113492 Bone2 RA | 0.4 |
| 112387 Match Control | 0.4 | 113493 Synovium2 RA | 0.4 |
| Crohns-M | | | |
| 112378 Crohns-M | 0.0 | 113494 Syn Fluid Cells RA | 0.0 |
| 112390 Match Control | 5.7 | 113499 Cartilage4 RA | 0.8 |
| Crohns-M | | | |
| 112726 Crohns-M | 100.0 | 113500 Bone4 RA | 1.2 |
| 112731 Match Control | 28.9 | 113501 Synovium4 RA | 0.6 |
| Crohns-M | | | |
| 112380 Ulcer Col-F | 30.8 | 113502 Syn Fluid Cells4 | 0.8 |
| | | RA | |
| 112734 Match Control | 0.0 | 113495 Cartilage3 RA | 0.8 |
| Ulcer Col-F | | | |
| 112384 Ulcer Col-F | 0.5 | 113496 Bone3 RA | 0.2 |
| 112737 Match Control | 28.9 | 113497 Synovium3 RA | 0.0 |
| Ulcer Col-F | | | |
| 112386 Ulcer Col-F | 0.5 | 113498 Syn Fluid Cells3 | 0.2 |
| | | RA | |
| 112738 Match Control | 0.0 | 117106 Normal Cartilage | 1.3 |
| Ulcer Col-F | | Rep20 | |
| 112381 Ulcer Col-M | 1.7 | 113663 Bone3 Normal | 0.0 |
| 112735 Match Control | 1.2 | 113664 Synovium3 Normal | 0.0 |
| Ulcer Col-M | | | |
| 112382 Ulcer Col-M | 0.0 | 113665 Syn Fluid Cells3 | 0.0 |
| | | Normal | |
| 112394 Match Control | 0.0 | 117107 Normal Cartilage | 1.8 |
| Ulcer Col-M | | Rep22 | |
| 112383 Ulcer Col-M | 12.0 | 113667 Bone4 Normal | 0.0 |
| 112736 Match Control | 0.0 | 113668 Synovium4 Normal | 0.7 |
| Ulcer Col-M | | | |
| 112423 Psoriasis-F | 11.3 | 113669 Syn Fluid Cells4 | 0.9 |
| | | Normal | |

TABLE GC
General_screening_panel_v1.4

| | Rel. Exp. (%) | | Rel. Exp. (%) |
|---|---|---|---|
| | Ag4214, Run | | Ag4214, Run |
| Tissue Name | 221254821 | Tissue Name | 221254821 |
| Adipose | 0.0 | Renal ca. TK-10 | 0.0 |
| Melanoma* | 0.9 | Bladder | 0.4 |
| Hs688(A).T | | | |
| Melanoma* | 0.0 | Gastric ca. (liver met.) | 0.0 |
| Hs688(B).T | | NCI-N87 | |
| Melanoma* M14 | 0.0 | Gastric ca. KATO III | 0.0 |
| Melanoma* | 2.9 | Colon ca. SW-948 | 0.0 |
| LOXIMVI | | | |
| Melanoma* SK- | 0.8 | Colon ca. SW480 | 0.0 |
| MEL-5 | | | |
| Squamous cell | 0.0 | Colon ca.* (SW480 | 0.0 |
| carcinoma SCC-4 | | met) SW620 | |
| Testis Pool | 100.0 | Colon ca. HT29 | 0.0 |
| Prostate ca.* (bone | 5.1 | Colon ca. HCT-116 | 0.0 |
| met) PC-3 | | | |
| Prostate Pool | 0.3 | Colon ca. CaCo-2 | 0.0 |
| Placenta | 0.0 | Colon cancer tissue | 0.0 |
| Uterus Pool | 0.0 | Colon ca. SW1116 | 0.0 |
| Ovarian ca. | 0.0 | Colon ca. Colo-205 | 0.0 |
| OVCAR-3 | | | |
| Ovarian ca. SK-OV- | 0.0 | Colon ca. SW-48 | 0.0 |
| 3 | | | |
| Ovarian ca. | 0.0 | Colon Pool | 0.0 |
| OVCAR-4 | | | |
| Ovarian ca. | 0.0 | Small Intestine Pool | 0.0 |
| OVCAR-5 | | | |
| Ovarian ca. IGROV- | 0.0 | Stomach Pool | 0.3 |
| 1 | | | |
| Ovarian ca. | 0.0 | Bone Marrow Pool | 0.0 |
| OVCAR-8 | | | |
| Ovary | 0.3 | Fetal Heart | 0.0 |
| Breast ca. MCF-7 | 0.0 | Heart Pool | 0.0 |
| Breast ca. MDA- | 0.0 | Lymph Node Pool | 0.0 |
| MB-231 | | | |
| Breast ca. BT 549 | 0.0 | Fetal Skeletal Muscle | 0.0 |
| Breast ca. T47D | 0.0 | Skeletal Muscle Pool | 0.2 |
| Breast ca. MDA-N | 0.0 | Spleen Pool | 1.8 |
| Breast Pool | 0.0 | Thymus Pool | 0.0 |
| Trachea | 0.0 | CNS cancer (glio/astro) | 1.7 |
| | | U87-MG | |
| Lung | 0.0 | CNS cancer (glio/astro) | 0.0 |
| | | U-118-MG | |
| Fetal Lung | 0.3 | CNS cancer | 0.0 |
| | | (neuro; met) SK-N-AS | |
| Lung ca. NCI-N417 | 0.0 | CNS cancer (astro) SF- | 0.4 |
| | | 539 | |
| Lung ca. LX-1 | 0.0 | CNS cancer (astro) | 0.5 |
| | | SNB-75 | |
| Lung ca. NCI-H146 | 0.0 | CNS cancer (glio) | 0.0 |
| | | SNB-19 | |
| Lung ca. SHP-77 | 0.7 | CNS cancer (glio) SF- | 11.5 |
| | | 295 | |
| Lung ca. A549 | 0.0 | Brain (Amygdala) Pool | 0.3 |
| Lung ca. NCI-H526 | 0.0 | Brain (cerebellum) | 0.4 |
| Lung ca. NCI-H23 | 0.4 | Brain (fetal) | 0.3 |
| Lung ca. NCI-H460 | 0.0 | Brain (Hippocampus) | 1.6 |
| | | Pool | |
| Lung ca. HOP-62 | 0.4 | Cerebral Cortex Pool | 0.8 |
| Lung ca. NCI-H522 | 0.0 | Brain (Substantia nigra) | 0.3 |
| | | Pool | |
| Liver | 0.2 | Brain (Thalamus) Pool | 1.6 |
| Fetal Liver | 0.3 | Brain (whole) | 0.3 |
| Liver ca. HepG2 | 0.0 | Spinal Cord Pool | 0.9 |
| Kidney Pool | 0.7 | Adrenal Gland | 0.0 |
| Fetal Kidney | 0.0 | Pituitary gland Pool | 0.0 |
| Renal ca. 786-0 | 0.0 | Salivary Gland | 0.0 |
| Renal ca. A498 | 0.0 | Thyroid (female) | 0.0 |
| Renal ca. ACHN | 0.0 | Pancreatic ca. | 0.0 |
| | | CAPAN2 | |
| Renal ca. UO-31 | 0.0 | Pancreas Pool | 0.4 |

TABLE GD
Panel 4.1D

| | Rel. Exp. (%) | | Rel. Exp. (%) |
|---|---|---|---|
| | Ag4214, Run | | Ag4214, Run |
| Tissue Name | 174261198 | Tissue Name | 174261198 |
| Secondary Th1 act | 0.0 | HUVEC IL-1beta | 22.5 |
| Secondary Th2 act | 0.0 | HUVEC IFN gamma | 13.8 |
| Secondary Tr1 act | 0.0 | HUVEC TNF alpha + IFN | 0.0 |
| | | gamma | |
| Secondary Th1 rest | 0.0 | HUVEC TNF alpha + IL4 | 3.7 |
| Secondary Th2 rest | 0.0 | HUVEC IL-11 | 11.1 |
| Secondary Tr1 rest | 0.0 | Lung Microvascular EC | 0.0 |
| | | none | |
| Primary Th1 act | 0.0 | Lung Microvascular EC | 1.3 |
| | | TNFalpha + IL-1 beta | |
| Primary Th2 act | 0.0 | Microvascular Dermal EC | 9.5 |
| | | none | |
| Primary Tr1 act | 0.0 | Microsvasular Dermal EC | 0.0 |
| | | TNFalpha + IL-1beta | |
| Primary Th1 rest | 0.0 | Bronchial epithelium | 0.0 |
| | | TNFalpha + IL1beta | |
| Primary Th2 rest | 0.0 | Small airway epithelium | 0.0 |
| | | none | |
| Primary Tr1 rest | 0.0 | Small airway epithelium | 0.0 |
| | | TNFalpha + IL-1beta | |
| CD45RA CD4 | 0.9 | Coronery artery SMC rest | 10.3 |
| lymphocyte act | | | |
| CD45RO CD4 | 0.0 | Coronery artery SMC | 15.4 |
| lymphocyte act | | TNFalpha + IL-1beta | |
| CD8 lymphocyte act | 0.0 | Astrocytes rest | 0.0 |
| Secondary CD8 | 0.0 | Astrocytes TNFalpha + | 0.0 |
| lymphocyte rest | | IL-1beta | |
| Secondary CD8 | 0.0 | KU-812 (Basophil) rest | 5.2 |
| lymphocyte act | | | |
| CD4 lymphocyte none | 4.6 | KU-812 (Basophil) | 0.0 |
| | | PMA/ionomycin | |
| 2ry Th1/Th2/Tr1_anti- | 0.0 | CCD1106 (Keratinocytes) | 0.0 |
| CD95 CH11 | | none | |
| LAK cells rest | 32.5 | CCD1106 (Keratinocytes) | 0.0 |
| | | TNFalpha + IL-1beta | |
| LAK cells IL-2 | 0.0 | Liver cirrhosis | 0.0 |
| LAK cells IL-2 + IL-12 | 0.0 | NCI-H292 none | 0.0 |
| LAK cells IL-2 + IFN | 0.0 | NCI-H292 IL-4 | 0.0 |
| gamma | | | |
| LAK cells IL-2 + IL-18 | 0.0 | NCI-H292 IL-9 | 0.0 |
| LAK cells | 5.7 | NCI-H292 IL-13 | 0.0 |
| PMA/ionomycin | | | |
| NK Cells IL-2 rest | 0.0 | NCI-H292 IFN gamma | 0.0 |
| Two Way MLR 3 day | 6.0 | HPAEC none | 16.6 |
| Two Way MLR 5 day | 6.2 | HPAEC TNF alpha + IL-1 | 3.2 |
| | | beta | |
| Two Way MLR 7 day | 0.0 | Lung fibroblast none | 0.0 |
| PBMC rest | 18.0 | Lung fibroblast TNF alpha + | 0.0 |
| | | IL-1 beta | |
| PBMC PWM | 0.0 | Lung fibroblast IL-4 | 0.0 |
| PBMC PHA-L | 0.0 | Lung fibroblast IL-9 | 0.0 |
| Ramos (B cell) none | 0.0 | Lung fibroblast IL-13 | 0.0 |
| Ramos (B cell) | 0.0 | Lung fibroblast IFN | 0.0 |
| ionomycin | | gamma | |
| B lymphocytes PWM | 0.0 | Dermal fibroblast | 0.0 |
| | | CCD1070 rest | |
| B lymphocytes CD40L | 0.0 | Dermal fibroblast | 0.0 |
| and IL-4 | | CCD1070 TNF alpha | |
| EOL-1 dbcAMP | 6.7 | Dermal fibroblast | 0.0 |
| | | CCD1070 IL-1 beta | |
| EOL-1 dbcAMP | 0.0 | Dermal fibroblast IFN | 3.3 |
| PMA/ionomycin | | gamma | |
| Dendritic cells none | 27.0 | Dermal fibroblast IL-4 | 3.0 |
| Dendritic cells LPS | 2.3 | Dermal Fibroblasts rest | 0.0 |
| Dendritic cells anti- | 13.3 | Neutrophils TNFa + LPS | 8.5 |
| CD40 | | | |
| Monocytes rest | 54.3 | Neutrophils rest | 20.4 |
| Monocytes LPS | 100.0 | Colon | 0.0 |
| Macrophages rest | 5.8 | Lung | 3.1 |
| Macrophages LPS | 0.0 | Thymus | 0.0 |
| HUVEC none | 22.2 | Kidney | 0.0 |
| HUVEC starved | 12.8 | | |

AI_comprehensive panel_v1.0 Summary: Ag4214 Highest expression of the CG101475-01 gene is detected in Crohn's disease sample (CT=29). In addition, significant expression of this gene is also seen in samples derived from normal lung samples, COPD lung, emphysema, atopic asthma, asthma, allergy, Crohn's disease (normal matched control and diseased), ulcerative colitis(normal matched control and diseased), psoriasis (normal matched control and diseased), bone (Orthoarthritis and matched control), OA synovium and rheumatoid arthritis cartilage Rep2. Therefore, therapeutic modulation of this gene product may ameliorate symptoms/conditions associated with autoimmune and inflammatory disorders including psoriasis, allergy, asthma, inflammatory bowel disease, rheumatoid arthritis and osteoarthritis

General_screening_panel_v1.4 Summary: Ag4214 Moderate level of expression of the CG101475-01 gene is detected only intestis (CT=31). Therefore, expression of this gene may be used to distinguish testis from other samples used in this panel. In addition, therapeutic modulation of this gene may be useful in the treatment of testis related diseases, including fertility and hypogonadism.

Low levels of expression of this gene is also detected in one of the CNS cancer cell line. Therefore, therapeutic modulation of this gene product may be beneficial in the treatment of CNS cancer.

Panel 4.1D Summary: Ag4214 Highest expression of the CG101475-01 gene is detected in LPS treated monocytes (CT=33). Low levels of expression of this gene is also detected in resting monocytes and LAK cells. Therefore, expression of this gene may be used to distinguish monocytes and LAK cells from other samples used in this panel. The expression of this gene in resting cells suggests that the protein encoded by this gene may be involved in normal immunological processes associated with immune homeostasis. In addition, expression of this gene in activated monocytes suggests that this gene may be involved in their function as antigen-presenting cells and antibodies or small molecule therapeutics that block the function of this membrane protein may be useful as anti-inflammatory therapeutics for the treatment of autoimmune and inflammatory diseases.

H. CG101475-02: Novel Plasma Membrane Protein Containing Lectin C-type Domain

Expression of gene CG101475-02 was assessed using the primer-probe set Ag6376, described in Table HA. Please note that CG101475-02 represents a full-length physical clone

TABLE HA

Probe Name Ag6376

| | | | Start | SEQ ID |
|---|---|---|---|---|
| Primers | Sequences | Length | Position | No |
| Forward | 5′-gatggctctgttccctctc-3′ | 19 | 171 | 171 |
| Probe | TET-5′-agtactaaagaacttgaccagatcaatgga-3′-TAMRA | 30 | 130 | 172 |
| Reverse | 5′-cagcgagaaatataaatatttcct-3′ | 24 | 80 | 173 |

I. CG102575-01 and CG102575-02: Novel ATPase Associated with Various Cellular Activities

Expression of gene CG102575-01 and CG102575-02 was assessed using the primer-probe set Ag4238, described in Table IA. Results of the RTQ-PCR runs are shown in Tables IB, IC, ID and IE. Please note that CG102575-02 represents a full-length physical clone of the CG102575-01 gene, validating the prediction of the gene sequence.

TABLE IA

puz,19/30 Probe Name Ag4238

| | | | Start | SEQ ID |
|---|---|---|---|---|
| Primers | Sequences | Length | Position | No |
| Forward | 5′-agatctggaggatacccagatc-3′ | 22 | 754 | 174 |
| Probe | TET-5′-ccaacatcaagaagtactccttataaacca-3′-TAMRA | 30 | 776 | 175 |
| Reverse | 5′-gcaaacatcactggctttatt-3′ | 22 | 824 | 176 |

TABLE IB
CNS_neurodegeneration_v1.0

| | Rel. Exp.(%) | | Rel. Exp.(%) |
|---|---|---|---|
| | Ag4238, Run | | Ag4238, Run |
| Tissue Name | 224065201 | Tissue Name | 224065201 |
| AD 1 Hippo | 10.2 | Control (Path) 3 | 8.2 |
| | | Temporal Ctx | |
| AD 2 Hippo | 22.8 | Control (Path) 4 | 31.0 |
| | | Temporal Ctx | |
| AD 3 Hippo | 14.4 | AD 1 Occipital | 32.1 |
| | | Ctx | |
| AD 4 Hippo | 8.2 | AD 2 Occipital | 0.0 |
| | | Ctx | |
| | | (Missing) | |
| AD 5 Hippo | 100.0 | AD 3 Occipital | 12.9 |
| | | Ctx | |
| AD 6 Hippo | 68.3 | AD 4 Occipital | 20.9 |
| | | Ctx | |
| Control 2 | 47.6 | AD 5 Occipital | 26.4 |
| Hippo | | Ctx | |
| Control 4 | 17.4 | AD 5 Occipital | 59.5 |
| Hippo | | Ctx | |
| Control (Path) | 9.8 | Control 1 | 6.2 |
| 3 Hippo | | Occipital Ctx | |
| AD 1 Temporal | 27.7 | Control 2 | 75.8 |
| Ctx | | Occipital Ctx | |
| AD 2 Temporal | 27.5 | Control 3 | 37.4 |
| Ctx | | Occipital Ctx | |
| AD 3 Temporal | 10.3 | Control 4 | 8.5 |
| Ctx | | Occipital Ctx | |
| AD 4 Temporal | 18.3 | Control (Path) 1 | 85.3 |
| Ctx | | Occipital Ctx | |
| AD 5 Inf | 86.5 | Control (Path) 2 | 12.1 |
| Temporal Ctx | | Occipital Ctx | |
| AD 5 Sup | 40.1 | Control (Path) 3 | 11.4 |
| Temporal Ctx | | Occipital Ctx | |
| AD 6 Inf | 59.5 | Control (Path) 4 | 19.6 |
| Temporal Ctx | | Occipital Ctx | |
| AD 6 Sup | 81.8 | Control 1 | 8.0 |
| Temporal Ctx | | Parietal Ctx | |
| Control 1 | 9.1 | Control 2 | 53.2 |
| Temporal Ctx | | Parietal Ctx | |
| Control 2 | 48.0 | Control 3 | 17.7 |
| Temporal Ctx | | Parietal Ctx | |
| Control 3 | 29.3 | Control (Path) 1 | 81.8 |
| Temporal Ctx | | Parietal Ctx | |
| Control 3 | 9.4 | Control (Path) 2 | 30.1 |
| Temporal Ctx | | Parietal Ctx | |
| Control (Path) 1 | 51.8 | Control (Path) 3 | 8.5 |
| Temporal Ctx | | Parietal Ctx | |
| Control (Path) 2 | 51.8 | Control (Path) 4 | 52.5 |
| Temporal Ctx | | Parietal Ctx | |

TABLE IC
General_screening_panel_v1.4

| | Rel. Exp. (%) | | Rel. Exp. (%) |
|---|---|---|---|
| | Ag4238, Run | | Ag4238, Run |
| Tissue Name | 222026508 | Tissue Name | 222026508 |
| Adipose | 12.2 | Renal ca. TK-10 | 24.3 |
| Melanoma* | 24.5 | Bladder | 25.0 |
| Hs688(A).T | | | |
| Melanoma* | 16.3 | Gastric ca. (liver met.) | 75.8 |
| Hs688(B).T | | NCI-N87 | |
| Melanoma* M14 | 35.8 | Gastric ca. KATO III | 60.7 |
| Melanoma* | 40.6 | Colon ca. SW-948 | 18.9 |
| LOXIMVI | | | |
| Melanoma* SK- | 32.1 | Colon ca. SW480 | 26.8 |
| MEL-5 | | | |
| Squamous cell | 29.7 | Colon ca.* (SW480 | 12.2 |
| carcinoma SCC-4 | | met) SW620 | |
| Testis Pool | 20.0 | Colon ca. HT29 | 18.7 |
| Prostate ca.* (bone | 27.5 | Colon ca. HCT-116 | 95.3 |
| met) PC-3 | | | |
| Prostate Pool | 10.7 | Colon ca. CaCo-2 | 25.7 |
| Placenta | 2.6 | Colon cancer tissue | 13.5 |
| Uterus Pool | 8.4 | Colon ca. SW1116 | 3.5 |
| Ovarian ca. | 29.1 | Colon ca. Colo-205 | 3.6 |
| OVCAR-3 | | | |
| Ovarian ca. SK-OV- | 69.7 | Colon ca. SW-48 | 3.0 |
| 3 | | | |
| Ovarian ca. | 11.1 | Colon Pool | 37.9 |
| OVCAR-4 | | | |
| Ovarian ca. | 25.5 | Small Intestine Pool | 30.8 |
| OVCAR-5 | | | |
| Ovarian ca. IGROV- | 12.6 | Stomach Pool | 16.6 |
| 1 | | | |
| Ovarian ca. | 7.8 | Bone Marrow Pool | 16.8 |
| OVCAR-8 | | | |
| Ovary | 7.9 | Fetal Heart | 24.3 |
| Breast ca. MCF-7 | 13.4 | Heart Pool | 13.7 |
| Breast ca. MDA- | 55.1 | Lymph Node Pool | 38.7 |
| MB-231 | | | |
| Breast ca. BT 549 | 100.0 | Fetal Skeletal Muscle | 11.1 |
| Breast ca. T47D | 40.1 | Skeletal Muscle Pool | 11.8 |
| Breast ca. MDA-N | 28.1 | Spleen Pool | 19.5 |
| Breast Pool | 38.2 | Thymus Pool | 25.7 |
| Trachea | 9.5 | CNS cancer (glio/astro) | 34.2 |
| | | U87-MG | |
| Lung | 6.1 | CNS cancer (glio/astro) | 47.0 |
| | | U-118-MG | |
| Fetal Lung | 20.0 | CNS cancer | 59.9 |
| | | (neuro; met) SK-N-AS | |
| Lung ca. NCI-N417 | 5.4 | CNS cancer (astro) SF- | 11.2 |
| | | 539 | |
| Lung ca. LX-1 | 24.0 | CNS cancer (astro) | 35.1 |
| | | SNB-75 | |
| Lung ca. NCI-H146 | 12.1 | CNS cancer (glio) | 13.2 |
| | | SNB-19 | |
| Lung ca. SHP-77 | 31.0 | CNS cancer (glio) SF- | 53.2 |
| | | 295 | |
| Lung ca. A549 | 31.0 | Brain (Amygdala) Pool | 11.7 |
| Lung ca. NCI-H526 | 7.3 | Brain (cerebellum) | 28.5 |
| Lung ca. NCI-H23 | 54.7 | Brain (fetal) | 37.4 |
| Lung ca. NCI-H460 | 42.6 | Brain (Hippocampus) | 11.0 |
| | | Pool | |
| Lung ca. HOP-62 | 17.6 | Cerebral Cortex Pool | 16.7 |
| Lung ca. NCI-H522 | 36.9 | Brain (Substantia nigra) | 8.8 |
| | | Pool | |
| Liver | 0.8 | Brain (Thalamus) Pool | 21.3 |
| Fetal Liver | 28.7 | Brain (whole) | 13.0 |
| Liver ca. HepG2 | 11.3 | Spinal Cord Pool | 9.0 |
| Kidney Pool | 53.6 | Adrenal Gland | 14.2 |
| Fetal Kidney | 37.6 | Pituitary gland Pool | 5.3 |
| Renal ca. 786-0 | 18.2 | Salivary Gland | 5.4 |
| Renal ca. A498 | 12.7 | Thyroid (female) | 4.3 |
| Renal ca. ACHN | 12.2 | Pancreatic ca. | 0.0 |
| | | CAPAN2 | |
| Renal ca. UO-31 | 21.9 | Pancreas Pool | 28.1 |

TABLE ID
Panel 4.1D

| | Rel. Exp. (%) | | Rel. Exp. (%) |
|---|---|---|---|
| | Ag4238, Run | | Ag4238, Run |
| Tissue Name | 175226771 | Tissue Name | 175226771 |
| Secondary Th1 act | 64.2 | HUVEC IL-1beta | 21.8 |
| Secondary Th2 act | 52.1 | HUVEC IFN gamma | 19.5 |
| Secondary Tr1 act | 34.9 | HUVEC TNF alpha + IFN | 9.1 |
| | | gamma | |
| Secondary Th1 rest | 10.2 | HUVEC TNF alpha + IL4 | 20.2 |
| Secondary Th2 rest | 7.8 | HUVEC IL-11 | 22.5 |
| Secondary Tr1 rest | 8.5 | Lung Microvascular EC | 28.3 |
| | | none | |
| Primary Th1 act | 100.0 | Lung Microvascular EC | 9.5 |
| | | TNFalpha + IL-1 beta | |
| Primary Th2 act | 62.4 | Microvascular Dermal EC | 21.0 |
| | | none | |
| Primary Tr1 act | 62.4 | Microsvasular Dermal EC | 11.0 |
| | | TNFalpha + IL-1beta | |
| Primary Th1 rest | 12.5 | Bronchial epithelium | 10.4 |
| | | TNFalpha + IL1beta | |
| Primary Th2 rest | 11.2 | Small airway epithelium | 3.0 |
| | | none | |
| Primary Tr1 rest | 24.0 | Small airway epithelium | 6.2 |
| | | TNFalpha + IL-1beta | |
| CD45RA CD4 | 25.2 | Coronery artery SMC rest | 7.3 |
| lymphocyte act | | | |
| CD45RO CD4 | 49.0 | Coronery artery SMC | 6.2 |
| lymphocyte act | | TNFalpha + IL-1beta | |
| CD8 lymphocyte act | 37.6 | Astrocytes rest | 3.3 |
| Secondary CD8 | 39.5 | Astrocytes TNFalpha + | 3.8 |
| lymphocyte rest | | IL-1beta | |
| Secondary CD8 | 19.3 | KU-812 (Basophil) rest | 62.9 |
| lymphocyte act | | | |
| CD4 lymphocyte none | 12.4 | KU-812 (Basophil) | 79.6 |
| | | PMA/ionomycin | |
| 2ry Th1/Th2/Tr1_anti- | 14.3 | CCD1106 (Keratinocytes) | 23.3 |
| CD95 CH11 | | none | |
| LAK cells rest | 19.8 | CCD1106 (Keratinocytes) | 14.1 |
| | | TNFalpha + IL-1beta | |
| LAK cells IL-2 | 43.8 | Liver cirrhosis | 4.2 |
| LAK cells IL-2 + IL-12 | 21.8 | NCI-H292 none | 9.3 |
| LAK cells IL-2 + IFN | 20.3 | NCI-H292 IL-4 | 20.2 |
| gamma | | | |
| LAK cells IL-2 + IL-18 | 28.1 | NCI-H292 IL-9 | 22.1 |
| LAK cells | 9.2 | NCI-H292 IL-13 | 16.3 |
| PMA/ionomycin | | | |
| NK Cells IL-2 rest | 53.2 | NCI-H292 IFN gamma | 15.1 |
| Two Way MLR 3 day | 24.7 | HPAEC none | 13.8 |
| Two Way MLR 5 day | 27.2 | HPAEC TNF alpha + IL-1 | 14.2 |
| | | beta | |
| Two Way MLR 7 day | 24.3 | Lung fibroblast none | 10.4 |
| PBMC rest | 4.8 | Lung fibroblast TNF alpha + | 5.4 |
| | | IL-1 beta | |
| PBMC PWM | 27.7 | Lung fibroblast IL-4 | 6.0 |
| PBMC PHA-L | 24.0 | Lung fibroblast IL-9 | 13.0 |
| Ramos (B cell) none | 53.6 | Lung fibroblast IL-13 | 11.8 |
| Ramos (B cell) | 45.7 | Lung fibroblast IFN | 11.3 |
| ionomycin | | gamma | |
| B lymphocytes PWM | 35.6 | Dermal fibroblast | 52.9 |
| | | CCD1070 rest | |
| B lymphocytes CD40L | 21.0 | Dermal fibroblast | 53.6 |
| and IL-4 | | CCD1070 TNF alpha | |
| EOL-1 dbcAMP | 41.8 | Dermal fibroblast | 23.0 |
| | | CCD1070 IL-1 beta | |
| EOL-1 dbcAMP | 24.5 | Dermal fibroblast IFN | 13.1 |
| PMA/ionomycin | | gamma | |
| Dendritic cells none | 10.4 | Dermal fibroblast IL-4 | 22.1 |
| Dendritic cells LPS | 4.6 | Dermal Fibroblasts rest | 6.3 |
| Dendritic cells anti- | 10.6 | Neutrophils TNFa + LPS | 0.0 |
| CD40 | | | |
| Monocytes rest | 7.3 | Neutrophils rest | 2.6 |
| Monocytes LPS | 6.5 | Colon | 4.0 |
| Macrophages rest | 10.4 | Lung | 7.2 |
| Macrophages LPS | 2.0 | Thymus | 21.5 |
| HUVEC none | 18.9 | Kidney | 24.0 |
| HUVEC starved | 24.7 | | |

TABLE IE
general oncology screening panel_v_2.4

| | Rel. Exp. (%) | | Rel. Exp. (%) |
|---|---|---|---|
| | Ag4238, Run | | Ag4238, Run |
| Tissue Name | 268664314 | Tissue Name | 268664314 |
| Colon cancer 1 | 16.0 | Bladder cancer | 0.0 |
| | | NAT 2 | |
| Colon cancer | 4.0 | Bladder cancer | 0.0 |
| NAT 1 | | NAT 3 | |
| Colon cancer 2 | 13.3 | Bladder cancer | 3.4 |
| | | NAT 4 | |
| Colon cancer | 9.5 | Adenocarcinoma of | 16.3 |
| NAT 2 | | the prostate 1 | |
| Colon cancer 3 | 14.8 | Adenocarcinoma of | 1.7 |
| | | the prostate 2 | |
| Colon cancer | 14.5 | Adenocarcinoma of | 8.9 |
| NAT 3 | | the prostate 3 | |
| Colon malignant | 37.4 | Adenocarcinoma of | 25.7 |
| cancer 4 | | the prostate 4 | |
| Colon normal | 1.9 | Prostate cancer | 1.6 |
| adjacent tissue | | NAT 5 | |
| 4 | | | |
| Lung cancer 1 | 9.2 | Adenocarcinoma of | 1.1 |
| | | the prostate 6 | |
| Lung NAT 1 | 0.0 | Adenocarcinoma of | 3.0 |
| | | the prostate 7 | |
| Lung cancer 2 | 100.0 | Adenocarcinoma of | 0.8 |
| | | the prostate 8 | |
| Lung NAT 2 | 1.6 | Adenocarcinoma of | 10.6 |
| | | the prostate 9 | |
| Squamous cell | 26.4 | Prostate cancer | 0.0 |
| carcinoma 3 | | NAT 10 | |
| Lung NAT 3 | 0.0 | Kidney cancer 1 | 18.2 |
| metastatic | 15.2 | Kidney NAT 1 | 13.5 |
| melanoma 1 | | | |
| Melanoma 2 | 1.5 | Kidney cancer 2 | 30.1 |
| Melanoma 3 | 1.9 | Kidney NAT 2 | 12.6 |
| metastatic | 24.0 | Kidney cancer 3 | 27.0 |
| melanoma 4 | | | |
| metastatic | 38.4 | Kidney NAT 3 | 7.6 |
| melanoma 5 | | | |
| Bladder cancer | 0.0 | Kidney cancer 4 | 7.3 |
| 1 | | | |
| Bladder cancer | 0.0 | Kidney NAT 4 | 3.0 |
| NAT 1 | | | |
| Bladder cancer | 1.9 | | |
| 2 | | | |

CNS_neurodegeneration_v1.0 Summary: Ag4238 This panel does not show differential expression of this gene in Alzheimer's disease. However, this expression profile confirms the presence of this gene in the brain. Please see Panel 1.4 for discussion of this gene in the central nervous system.

General_screening_panel_v1.4 Summary: Ag4238 Highest expression of this gene is seen in a breast cancer cell line (CT=30.2). This gene is widely expressed in this panel, with moderate expression seen in brain, colon, gastric, lung, breast, ovarian, and melanoma cancers. This expression profile suggests a role for this gene product in cell survival and proliferation. Modulation of this gene product may be useful in the treatment of cancer.

Among tissues with metabolic function, this gene is expressed at low but significant levels in pituitary, adipose, adrenal gland, pancreas, thyroid, and adult and fetal skeletal muscle, heart, and liver. This widespread expression among these tissues suggests that this gene product may play a role in normal neuroendocrine and metabolic function and that disregulated expression of this gene may contribute to neuroendocrine disorders or metabolic diseases, such as obesity and diabetes.

Interestingly, this gene is expressed at much higher levels in fetal (CT=32) when compared to adult liver (CT=37). This observation suggests that expression of this gene can be used to distinguish fetal from adult liver. In addition, the relative overexpression of this gene in fetal liver suggests that the protein product may enhance liver growth or development in the fetus and thus may also act in a regenerative capacity in the adult. Therefore, therapeutic modulation of the protein encoded by this gene could be useful in treatment of liver related diseases.

This gene is also expressed at low but significant levels in the CNS, including the hippocampus, thalamus, substantia nigra, amygdala, cerebellum and cerebral cortex. Therefore, therapeutic modulation of the expression or function of this gene may be useful in the treatment of neurologic disorders, such as Alzheimer's disease, Parkinson's disease, schizophrenia, multiple sclerosis, stroke and epilepsy.

Panel 4.1D Summary: Ag4238 Highest expression of this gene is seen in acutely activated Th1 cells (CT=33.2). In addition, this gene is expressed in a wide range of cell types including activated T cells, LAK cells, dermal fibroblasts, and basophils. This pattern of expression is in agreement with the expression profile in General_screening_panel_v1.4 and also suggests a role for the gene product in cell survival and proliferation. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

general oncology screening panel_v_{—}2.4 Summary: Ag4238 This gene is widely expressed in this panel, with highest expression in lung cancer (CT=32.6). In addition, this gene is more highly expressed in lung and kidney cancer than in the corresponding normal adjacent tissue. In addition, significant expression of this gene is also associated with colon, and prostate cancer and also with melanoma. Thus, expression of this gene could be used as a marker of these cancers. Furthemore, therapeutic modulation of the expression or function of this gene product may be useful in the treatment of lung and kidney cancer.

J. CG102615-01: mat8

Expression of gene CG102615-01 was assessed using the primer-probe set Ag2173, described in Table JA. Results of the RTQ-PCR runs are shown in Tables JB, JC, JD and JE.

TABLE JA

uz,17/29 Probe Name Ag2173

| | | | Start | SEQ ID |
|---|---|---|---|---|
| Primers | Sequences | Length | Position | No |
| Forward | 5′-acttgaactccaggatggaatt-3′ | 22 | 332 | 177 |
| Probe | TET-5′-cttcctcctctgctgggactcctttg-3′-TAMRA | 26 | 354 | 178 |
| Reverse | 5′-cttgcgagaggtgagatgag-3′ | 20 | 391 | 179 |

TABLE JB
Panel 1.3D

| | Rel. Exp. (%) | | Rel. Exp. (%) |
|---|---|---|---|
| | Ag2173, Run | | Ag2173, Run |
| Tissue Name | 165750872 | Tissue Name | 165750872 |
| Liver adenocarcinoma | 8.6 | Kidney (fetal) | 0.6 |
| Pancreas | 0.3 | Renal ca. 786-0 | 0.0 |
| Pancreatic ca. CAPAN 2 | 1.8 | Renal ca. A498 | 0.1 |
| Adrenal gland | 0.1 | Renal ca. RXF 393 | 0.0 |
| Thyroid | 0.1 | Renal ca. ACHN | 0.0 |
| Salivary gland | 3.6 | Renal ca. UO-31 | 0.2 |
| Pituitary gland | 0.3 | Renal ca. TK-10 | 0.0 |
| Brain (fetal) | 0.0 | Liver | 0.0 |
| Brain (whole) | 0.0 | Liver (fetal) | 0.0 |
| Brain (amygdala) | 0.1 | Liver ca. (hepatoblast) | 0.6 |
| | | HepG2 | |
| Brain (cerebellum) | 0.0 | Lung | 4.7 |
| Brain (hippocampus) | 0.2 | Lung (fetal) | 3.8 |
| Brain (substantia nigra) | 0.1 | Lung ca. (small cell) | 1.3 |
| | | LX-1 | |
| Brain (thalamus) | 0.1 | Lung ca. (small cell) | 1.5 |
| | | NCI-H69 | |
| Cerebral Cortex | 0.5 | Lung ca. (s. cell var.) | 0.0 |
| | | SHP-77 | |
| Spinal cord | 0.8 | Lung ca. (large | 0.0 |
| | | cell) NCI-H460 | |
| glio/astro U87-MG | 0.0 | Lung ca. (non-sm. cell) | 0.0 |
| | | A549 | |
| glio/astro U-118-MG | 0.1 | Lung ca. (non-s. cell) | 0.0 |
| | | NCI-H23 | |
| astrocytoma SW1783 | 0.0 | Lung ca. (non-s. cell) | 0.0 |
| | | HOP-62 | |
| neuro*; met SK-N-AS | 0.0 | Lung ca. (non-s. cl) | 0.0 |
| | | NCI-H522 | |
| astrocytoma SF-539 | 0.1 | Lung ca. (squam.) SW | 0.3 |
| | | 900 | |
| astrocytoma SNB-75 | 0.2 | Lung ca. (squam.) NCI- | 5.2 |
| | | H596 | |
| glioma SNB-19 | 0.0 | Mammary gland | 3.7 |
| glioma U251 | 0.0 | Breast ca.* (pl. ef) | 7.9 |
| | | MCF-7 | |
| glioma SF-295 | 0.0 | Breast ca.* (pl. ef) | 0.0 |
| | | MDA-MB-231 | |
| Heart (fetal) | 0.0 | Breast ca.* (pl. ef) | 1.0 |
| | | T47D | |
| Heart | 0.1 | Breast ca. BT-549 | 0.0 |
| Skeletal muscle (fetal) | 0.6 | Breast ca. MDA-N | 0.0 |
| Skeletal muscle | 0.1 | Ovary | 1.0 |
| Bone marrow | 0.1 | Ovarian ca. OVCAR-3 | 0.2 |
| Thymus | 1.6 | Ovarian ca. OVCAR-4 | 0.1 |
| Spleen | 0.1 | Ovarian ca. OVCAR-5 | 1.7 |
| Lymph node | 0.0 | Ovarian ca. OVCAR-8 | 0.0 |
| Colorectal | 100.0 | Ovarian ca. IGROV-1 | 0.0 |
| Stomach | 5.2 | Ovarian ca.* (ascites) | 0.0 |
| | | SK-OV-3 | |
| Small intestine | 0.5 | Uterus | 0.1 |
| Colon ca. SW480 | 0.0 | Placenta | 3.1 |
| Colon ca.* | 0.1 | Prostate | 12.7 |
| SW620 (SW480 met) | | | |
| Colon ca. HT29 | 2.9 | Prostate ca.* (bone | 0.0 |
| | | met) PC-3 | |
| Colon ca. HCT-116 | 0.2 | Testis | 0.0 |
| Colon ca. CaCo-2 | 3.3 | Melanoma Hs688(A).T | 0.0 |
| Colon ca. | 6.1 | Melanoma* (met) | 0.0 |
| tissue (ODO3866) | | Hs688(B).T | |
| Colon ca. HCC-2998 | 1.3 | Melanoma UACC-62 | 5.1 |
| Gastric ca.* (liver met) | 15.4 | Melanoma M14 | 5.4 |
| NCI-N87 | | | |
| Bladder | 6.1 | Melanoma LOX IMVI | 0.0 |
| Trachea | 21.6 | Melanoma* (met) SK- | 0.0 |
| | | MEL-5 | |
| Kidney | 0.5 | Adipose | 0.5 |

TABLE JC
Panel 2D

| | Rel. Exp. (%) Ag2173, | | Rel. Exp. (%) Ag2173, |
|---|---|---|---|
| Tissue Name | Run 162309316 | Tissue Name | Run 162309316 |
| Normal Colon | 94.6 | Kidney Margin 8120608 | 0.3 |
| CC Well to Mod Diff | 9.3 | Kidney Cancer 8120613 | 0.0 |
| (ODO3866) | | | |
| CC Margin (ODO3866) | 33.2 | Kidney Margin 8120614 | 0.6 |
| CC Gr.2 rectosigmoid | 15.7 | Kidney Cancer 9010320 | 0.8 |
| (ODO3868) | | | |
| CC Margin (ODO3868) | 2.3 | Kidney Margin 9010321 | 0.3 |
| CC Mod Diff (ODO3920) | 13.7 | Normal Uterus | 0.0 |
| CC Margin (ODO3920) | 40.1 | Uterus Cancer 064011 | 4.6 |
| CC Gr.2 ascend colon | 52.9 | Normal Thyroid | 0.1 |
| (ODO3921) | | | |
| CC Margin (ODO3921) | 36.6 | Thyroid Cancer 064010 | 0.2 |
| CC from Partial Hepatectomy | 13.1 | Thyroid Cancer A302152 | 0.4 |
| (ODO4309) Mets | | | |
| Liver Margin (ODO4309) | 0.1 | Thyroid Margin A302153 | 0.0 |
| Colon mets to lung | 11.3 | Normal Breast | 9.9 |
| (OD04451-01) | | | |
| Lung Margin (OD04451-02) | 8.7 | Breast Cancer (OD04566) | 10.7 |
| Normal Prostate 6546-1 | 100.0 | Breast Cancer (OD04590- | 37.9 |
| | | 01) | |
| Prostate Cancer (OD04410) | 31.2 | Breast Cancer Mets | 58.2 |
| | | (OD04590-03) | |
| Prostate Margin (OD04410) | 32.3 | Breast Cancer Metastasis | 14.8 |
| | | (OD04655-05) | |
| Prostate Cancer (OD04720- | 11.1 | Breast Cancer 064006 | 11.0 |
| 01) | | | |
| Prostate Margin (OD04720- | 22.2 | Breast Cancer 1024 | 20.4 |
| 02) | | | |
| Normal Lung 061010 | 16.8 | Breast Cancer 9100266 | 26.6 |
| Lung Met to Muscle | 0.1 | Breast Margin 9100265 | 10.6 |
| (ODO4286) | | | |
| Muscle Margin (ODO4286) | 0.1 | Breast Cancer A209073 | 27.0 |
| Lung Malignant Cancer | 33.0 | Breast Margin A209073 | 9.3 |
| (OD03126) | | | |
| Lung Margin (OD03126) | 13.5 | Normal Liver | 0.0 |
| Lung Cancer (OD04404) | 64.6 | Liver Cancer 064003 | 0.0 |
| Lung Margin (OD04404) | 9.9 | Liver Cancer 1025 | 0.0 |
| Lung Cancer (OD04565) | 34.4 | Liver Cancer 1026 | 0.5 |
| Lung Margin (OD04565) | 5.5 | Liver Cancer 6004-T | 0.0 |
| Lung Cancer (OD04237-01) | 4.0 | Liver Tissue 6004-N | 0.1 |
| Lung Margin (OD04237-02) | 10.7 | Liver Cancer 6005-T | 0.4 |
| Ocular Mel Met to Liver | 0.0 | Liver Tissue 6005-N | 0.0 |
| (ODO4310) | | | |
| Liver Margin (ODO4310) | 0.0 | Normal Bladder | 11.0 |
| Melanoma Mets to Lung | 5.1 | Bladder Cancer 1023 | 15.5 |
| (OD04321) | | | |
| Lung Margin (OD04321) | 9.5 | Bladder Cancer A302173 | 20.4 |
| Normal Kidney | 0.6 | Bladder Cancer | 84.7 |
| | | (OD04718-01) | |
| Kidney Ca, Nuclear grade 2 | 0.5 | Bladder Normal Adjacent | 0.3 |
| (OD04338) | | (OD04718-03) | |
| Kidney Margin (OD04338) | 1.4 | Normal Ovary | 0.5 |
| Kidney Ca Nuclear grade 1/2 | 0.0 | Ovarian Cancer 064008 | 5.5 |
| (OD04339) | | | |
| Kidney Margin (OD04339) | 0.5 | Ovarian Cancer | 3.0 |
| | | (OD04768-07) | |
| Kidney Ca, Clear cell type | 0.0 | Ovary Margin | 0.0 |
| (OD04340) | | (OD04768-08) | |
| Kidney Margin (OD04340) | 0.7 | Normal Stomach | 18.3 |
| Kidney Ca, Nuclear grade 3 | 0.1 | Gastric Cancer 9060358 | 3.3 |
| (OD04348) | | | |
| Kidney Margin (OD04348) | 0.4 | Stomach Margin 9060359 | 22.5 |
| Kidney Cancer (OD04622-01) | 0.1 | Gastric Cancer 9060395 | 24.8 |
| Kidney Margin (OD04622- | 1.1 | Stomach Margin 9060394 | 20.6 |
| 03) | | | |
| Kidney Cancer (OD04450-01) | 0.0 | Gastric Cancer 9060397 | 27.2 |
| Kidney Margin (OD04450- | 0.9 | Stomach Margin 9060396 | 30.4 |
| 03) | | | |
| Kidney Cancer 8120607 | 0.0 | Gastric Cancer 064005 | 20.6 |

TABLE JD
Panel 4D

| | Rel. Exp. (%) Ag2173, | | Rel. Exp. (%) Ag2173, |
|---|---|---|---|
| Tissue Name | Run 162292974 | Tissue Name | Run 162292974 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC | 0.0 |
| | | TNF alpha + IL-1beta | |
| Primary Th2 act | 0.0 | Microvascular Dermal EC | 0.0 |
| | | none | |
| Primary Tr1 act | 0.0 | Microsvasular Dermal EC | 0.0 |
| | | TNF alpha + IL-1beta | |
| Primary Th1 rest | 0.1 | Bronchial epithelium | 61.6 |
| | | TNF alpha + IL1beta | |
| Primary Th2 rest | 0.0 | Small airway epithelium none | 23.2 |
| Primary Tr1 rest | 0.0 | Small airway epithelium | 76.8 |
| | | TNF alpha + IL-1beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNF alpha + IL-1beta | |
| CD8 lymphocyte act | 0.0 | Astrocytes rest | 0.0 |
| Secondary CD8 | 0.0 | Astrocytes TNF alpha + IL- | 0.0 |
| lymphocyte rest | | 1beta | |
| Secondary CD8 | 0.0 | KU-812 (Basophil) rest | 0.3 |
| lymphocyte act | | | |
| CD4 lymphocyte none | 0.0 | KU-812 (Basophil) | 0.3 |
| | | PMA/ionomycin | |
| 2ry Th1/Th2/Tr1_anti- | 0.0 | CCD1106 (Keratinocytes) | 21.2 |
| CD95 CH11 | | none | |
| LAK cells rest | 0.0 | CCD1106 (Keratinocytes) | 20.4 |
| | | TNF alpha + IL-1beta | |
| LAK cells IL-2 | 0.0 | Liver cirrhosis | 0.2 |
| LAK cells IL-2 + IL-12 | 0.0 | Lupus kidney | 0.3 |
| LAK cells IL-2 + IFN | 0.0 | NCI-H292 none | 100.0 |
| gamma | | | |
| LAK cells IL-2 + IL-18 | 0.0 | NCI-H292 IL-4 | 60.3 |
| LAK cells | 0.0 | NCI-H292 IL-9 | 80.7 |
| PMA/ionomycin | | | |
| NK Cells IL-2 rest | 0.0 | NCI-H292 IL-13 | 42.9 |
| Two Way MLR 3 day | 0.0 | NCI-H292 IFN gamma | 62.9 |
| Two Way MLR 5 day | 0.0 | HPAEC none | 0.0 |
| Two Way MLR 7 day | 0.0 | HPAEC TNF alpha + IL-1beta | 0.0 |
| PBMC rest | 0.0 | Lung fibroblast none | 0.0 |
| PBMC PWM | 0.0 | Lung fibroblast TNF alpha + | 0.0 |
| | | IL-1beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) ionomycin | 0.0 | Lung fibroblast IL-13 | 0.0 |
| B lymphocytes PWM | 0.0 | Lung fibroblast IFN gamma | 0.0 |
| B lymphocytes CD40L | 0.0 | Dermal fibroblast CCD1070 | 0.0 |
| and IL-4 | | rest | |
| EOL-1 dbcAMP | 0.0 | Dermal fibroblast CCD1070 | 0.0 |
| | | TNF alpha | |
| EOL-1 dbcAMP | 0.0 | Dermal fibroblast CCD1070 | 0.0 |
| PMA/ionomycin | | IL-1beta | |
| Dendritic cells none | 0.0 | Dermal fibroblast IFN gamma | 0.1 |
| Dendritic cells LPS | 0.0 | Dermal fibroblast IL-4 | 0.0 |
| Dendritic cells anti-CD40 | 0.1 | IBD Colitis 2 | 0.1 |
| Monycytes rest | 0.0 | IBD Crohn's | 0.1 |
| Monocytes LPS | 0.0 | Colon | 7.4 |
| Macrophages rest | 0.0 | Lung | 3.5 |
| Macrophages LPS | 0.0 | Thymus | 0.5 |
| HUVEC none | 0.0 | Kidney | 0.6 |
| HUVEC starved | 0.0 | | |

TABLE JE
Panel 5 Islet

| | Rel. Exp. (%) | | Rel. Exp. (%) |
|---|---|---|---|
| | Ag2173, Run | | Ag2173, Run |
| Tissue Name | 279370794 | Tissue Name | 279370794 |
| 97457_Patient- | 1.9 | 94709_Donor 2 AM - A_adipose | 0.0 |
| 02go_adipose | | | |
| 97476_Patient- | 0.0 | 94710_Donor 2 AM - B_adipose | 0.3 |
| 07sk_skeletal muscle | | | |
| 97477_Patient-07ut_uterus | 0.0 | 94711_Donor 2 AM - C_adipose | 0.0 |
| 97478_Patient- | 60.7 | 94712_Donor 2 AD - A_adipose | 0.0 |
| 07pl_placenta | | | |
| 99167_Bayer Patient 1 | 47.6 | 94713_Donor 2 AD - B_adipose | 2.1 |
| 97482_Patient-08ut_uterus | 1.2 | 94714_Donor 2 AD - C_adipose | 0.3 |
| 97483_Patient- | 69.3 | 94742_Donor 3 U - A_Mesenchymal | 0.0 |
| 08pl_placenta | | Stem Cells | |
| 97486_Patient- | 0.0 | 94743_Donor 3 U - B_Mesenchymal | 0.0 |
| 09sk_skeletal muscle | | Stem Cells | |
| 97487_Patient-09ut_uterus | 0.2 | 94730_Donor 3 AM - A_adipose | 1.1 |
| 97488_Patient- | 31.6 | 94731_Donor 3 AM - B_adipose | 0.7 |
| 09pl_placenta | | | |
| 97492_Patient-10ut_uterus | 2.7 | 94732_Donor 3 AM - C_adipose | 1.0 |
| 97493_Patient- | 73.7 | 94733_Donor 3 AD —A_adipose | 0.8 |
| 10pl_placenta | | | |
| 97495_Patient- | 0.6 | 94734_Donor 3 AD —B_adipose | 0.2 |
| 11go_adipose | | | |
| 97496_Patient- | 1.1 | 94735_Donor 3 AD —C_adipose | 1.0 |
| 11sk_skeletal muscle | | | |
| 97497_Patient-11ut_uterus | 1.0 | 77138_Liver_HepG2untreated | 100.0 |
| 97498_Patient- | 31.4 | 73556_Heart_Cardiac stromal cells | 0.0 |
| 11pl_placenta | | (primary) | |
| 97500_Patient- | 0.0 | 81735_Small Intestine | 61.6 |
| 12go_adipose | | | |
| 97501_Patient- | 0.4 | 72409_Kidney_Proximal Convoluted | 0.0 |
| 12sk_skeletal muscle | | Tubule | |
| 97502_Patient-12ut_uterus | 1.3 | 82685_Small intestine_Duodenum | 12.2 |
| 97503_Patient- | 68.8 | 90650_Adrenal_Adrenocortical | 0.0 |
| 12pl_placenta | | adenoma | |
| 94721_Donor 2 U — | 0.5 | 72410_Kidney_HRCE | 1.9 |
| A_Mesenchymal Stem | | | |
| Cells | | | |
| 94722_Donor 2 U — | 0.8 | 72411_Kidney_HRE | 0.6 |
| B_Mesenchymal Stem | | | |
| Cells | | | |
| 94723_Donor 2 U — | 0.3 | 73139_Uterus_Uterine smooth | 0.0 |
| C_Mesenchymal Stem | | muscle cells | |
| Cells | | | |

Panel 1.3D Summary: Ag2173 Highest expression of the CG102615-01 gene is detected in colorectal sample (CT=24). Therefore, expression of this gene may be used to distinguish this sample from other samples used in this panel. In addition, significant expression of this gene is seen in number of cancer cell lines including melanoma, ovarian, breast, lung, colon, pancreatic and liver cancer cell lines. The CG102615-01 gene codes for chloride conductane inducer protein MAT-8 precursor. MAT-8 is known to mediate chloride flow, affecting the membrane potential of the cell (Morrison et al., 1995, J. Biol. Chem. 270:2176-2182, PMID=7836447). Changes in membrane potential can affect tumor cell and associated smooth muscle cells (therefore tumor-induced vasculature) growth and motility. In this respect the expression of this gene in fetal muscle is an indication of a role in muscle growth/development. Therapeutic targeting of the CG102615-01 gene product with a monoclonal antibody is anticipated to limit or block the extent of tumor cell growth and motility and tumor associated angiogenesis, preferably in breast, ovarian bladder, lung tumors.

Among tissues with metabolic or endocrine function, this gene is expressed at high to moderate levels in pancreas, adipose, thyroid, pituitary gland, skeletal muscle, heart, and the gastrointestinal tract. Therefore, therapeutic modulation of the activity of this gene may prove useful in the treatment of endocrine/metabolically related diseases, such as obesity and diabetes.

In addition, this gene is expressed at low levels in all regions of the central nervous system examined, including amygdala, hippocampus, substantia nigra, thalamus, cerebellum, cerebral cortex, and spinal cord. Therefore, this gene may play a role in central nervous system disorders such as Alzheimer's disease, Parkinson's disease, epilepsy, multiple sclerosis, schizophrenia and depression.

Panel 2D Summary: Ag2173 Highest expression of the CG102615-01 gene is detected in normal prostate (CT=22.8). High expression of this gene is seen in normal and cancer samples derived from colon, prostate, lung, melanoma, uterus, thyroid, breast, liver, bladder, ovary, and stomach. Interestingly, expression of this gene is higher in ovarian, bladder, breast, uterine, and lung cancer samples as compared to their corresponding adjacent control samples. Therefore, therapeutic targeting of the CG102615-01 gene product with antibodies or small molecule inhibitors is anticipated to limit or block the extent of tumor cell growth and motility as well as tumor associated angiogenesis, preferably in ovarian, bladder, breast, uterine, and lung cancer.

Also the expression of this gene is decreased in kidney cancers compared to the normal adjacent tissues. Hence the protein product or fragments of this protein may be useful in the treatment of kidney cancer.

Panel 4D Summary: Ag2173 Highest expression of the CG102615-01 gene is detected in NCI-H292 cells (CT=23.8). High to moderate level of this gene is also found in lung derived cell types: small airway and bronchial epithelium treated with TNF-a and Il-1, lung fibroblast treated with IFN. This pattern of expression suggests a role for this gene in pathology of lung inflammatory dideases. Therefore therapeutic modulation of this gene product may be beneficial in the treatment of asthma, emphysema or lung infection.

Interestingly, high to low levels of expression of this gene is also seen in keratinocytes, basophils, IFN gamma treated dermal fibroblasts, liver cirrhosis, IBD colitis and Crohn's disease samples and normal tissues represented by colon, lung, thymus and kidney. Therefore, therapeutic modulation of this gene product may be beneficial in the treatment of autoimmune and inflammatory disease associated with these cell types and tissues including asthma, allergies, inflammatory bowel disease, lupus erythematosus, psoriasis, rheumatoid arthritis, and osteoarthritis.

Panel 5 Islet Summary: Ag2173 Highest expression of the CG102615-01 gene is detected in untreated liver HepG2 samples (CT=28.6). In addition, high to low levels of expression of this gene is also seen in islet cells, placenta, uterus, small intestine, kidney and adipose tissues. The CG102615-01 gene codes for chloride conductane inducer protein MAT-8 precursor. MAT-8 is known to mediate chloride flow, affecting the membrane potential of the cell (Morrison et al., 1995, J. Biol. Chem. 270:2176-2182, PMID=7836447). Since membrane potential is critical in the secretory process in the beta cell, therapeutic modulation of the activity of this gene may prove useful in enhancing insulin secretion in Type II diabetes.

K. CG102646-01: High Affinity Proline Permease Like

Expression of gene CG102646-01 was assessed using the primer-probe set Ag4241, described in Table KA.

TABLE KA

Probe Name Ag4241

| | | | Start | SEQ ID |
|---|---|---|---|---|
| Primers | Sequences | Length | Position | No |
| Forward | 5′-ggcagcaattatgagtacgatt-3′ | 22 | 1035 | 180 |
| Probe | TET-5′-tcccaattacttgtgacttcaagttca-3′ | 27 | 1000 | 181 |
| Reverse | 5′-tgcttcttcaccacgaattaa-3′ | 21 | 1108 | 182 |

L. CG102878-01 and CG102878-02: Hypothetical Transmembrane

Expression of gene CG102878-01 and CG102878-02 was assessed using the primer-probe set Ag4246, described in Table LA. Results of the RTQ-PCR runs are shown in Tables LB, LC, LD and LE. Please note that CG102878-02 represents a full-length physical clone of the CG102878-01 gene, validating the prediction of the gene sequence.

TABLE LA

Probe Name Ag4246

| | | | Start | SEQ ID |
|---|---|---|---|---|
| Primers | Sequences | Length | Position | No |
| Forward | 5′-cctccctggtagaggtcaac-3′ | 20 | 824 | 183 |
| Probe | TET-5′-ctactcagtgcccagcagccaggag-3′-TAMRA | 25 | 849 | 184 |
| Reverse | 5′-tgtctgcatgcagcctatg-3′ | 19 | 885 | 185 |

TABLE LB
CNS_neurodegeneration_v1.0

| | Rel. Exp. (%) Ag4246, Run | | Rel. Exp. (%) Ag4246, Run |
|---|---|---|---|
| Tissue Name | 224077627 | Tissue Name | 224077627 |
| AD 1 Hippo | 31.0 | Control (Path) 3 | 40.3 |
| | | Temporal Ctx | |
| AD 2 Hippo | 56.3 | Control (Path) 4 | 46.3 |
| | | Temporal Ctx | |
| AD 3 Hippo | 22.4 | AD 1 Occipital Ctx | 20.6 |
| AD 4 Hippo | 28.5 | AD 2 Occipital Ctx | 0.0 |
| | | (Missing) | |
| AD 5 Hippo | 79.0 | AD 3 Occipital Ctx | 21.3 |
| AD 6 Hippo | 76.3 | AD 4 Occipital Ctx | 31.6 |
| Control 2 Hippo | 65.5 | AD 5 Occipital Ctx | 83.5 |
| Control 4 Hippo | 49.0 | AD 6 Occipital Ctx | 41.5 |
| Control (Path) 3 Hippo | 35.4 | Control 1 Occipital Ctx | 20.2 |
| AD 1 Temporal Ctx | 30.1 | Control 2 Occipital Ctx | 92.0 |
| AD 2 Temporal Ctx | 36.3 | Control 3 Occipital Ctx | 70.7 |
| AD 3 Temporal Ctx | 14.3 | Control 4 Occipital Ctx | 28.7 |
| AD 4 Temporal Ctx | 28.1 | Control (Path) 1 | 100.0 |
| | | Occipital Ctx | |
| AD 5 Inf Temporal Ctx | 80.7 | Control (Path) 2 | 49.0 |
| | | Occipital Ctx | |
| AD 5 Sup Temporal | 66.4 | Control (Path) 3 | 36.1 |
| Ctx | | Occipital Ctx | |
| AD 6 Inf Temporal Ctx | 64.2 | Control (Path) 4 | 60.3 |
| | | Occipital Ctx | |
| AD 6 Sup Temporal | 78.5 | Control 1 Parietal Ctx | 24.8 |
| Ctx | | | |
| Control 1 Temporal | 35.4 | Control 2 Parietal Ctx | 83.5 |
| Ctx | | | |
| Control 2 Temporal | 68.3 | Control 3 Parietal Ctx | 34.4 |
| Ctx | | | |
| Control 3 Temporal | 41.2 | Control (Path) 1 | 71.2 |
| Ctx | | Parietal Ctx | |
| Control 3 Temporal | 32.3 | Control (Path) 2 | 52.5 |
| Ctx | | Parietal Ctx | |
| Control (Path) 1 | 55.5 | Control (Path) 3 | 32.5 |
| Temporal Ctx | | Parietal Ctx | |
| Control (Path) 2 | 67.8 | Control (Path) 4 | 76.8 |
| Temporal Ctx | | Parietal Ctx | |

TABLE LC
General_screening_panel_v1.4

| | Rel. Exp. (%) Ag4246, | | Rel. Exp. (%) Ag4246, |
|---|---|---|---|
| Tissue Name | Run 222018714 | Tissue Name | Run 222018714 |
| Adipose | 1.1 | Renal ca. TK-10 | 3.5 |
| Melanoma* | 4.8 | Bladder | 5.7 |
| Hs688(A).T | | | |
| Melanoma* | 2.8 | Gastric ca. (liver met.) | 12.8 |
| Hs688(B).T | | NCI-N87 | |
| Melanoma* M14 | 3.1 | Gastric ca. KATO III | 12.7 |
| Melanoma* LOXIMVI | 3.3 | Colon ca. SW-948 | 6.2 |
| Melanoma* SK-MEL-5 | 3.2 | Colon ca. SW480 | 12.0 |
| Squamous cell | 1.6 | Colon ca.* (SW480 met) | 7.5 |
| carcinoma SCC-4 | | SW620 | |
| Testis Pool | 2.5 | Colon ca. HT29 | 7.1 |
| Prostate ca.* (bone met) | 1.6 | Colon ca. HCT-116 | 8.4 |
| PC-3 | | | |
| Prostate Pool | 2.7 | Colon ca. CaCo-2 | 10.6 |
| Placenta | 2.6 | Colon cancer tissue | 4.8 |
| Uterus Pool | 0.8 | Colon ca. SW1116 | 9.7 |
| Ovarian ca. OVCAR-3 | 18.7 | Colon ca. Colo-205 | 4.0 |
| Ovarian ca. SK-OV-3 | 14.1 | Colon ca. SW-48 | 5.0 |
| Ovarian ca. OVCAR-4 | 3.3 | Colon Pool | 4.9 |
| Ovarian ca. OVCAR-5 | 27.5 | Small Intestine Pool | 3.4 |
| Ovarian ca. IGROV-1 | 14.9 | Stomach Pool | 1.9 |
| Ovarian ca. OVCAR-8 | 14.2 | Bone Marrow Pool | 1.1 |
| Ovary | 3.3 | Fetal Heart | 3.8 |
| Breast ca. MCF-7 | 5.3 | Heart Pool | 2.4 |
| Breast ca. MDA-MB- | 9.4 | Lymph Node Pool | 3.2 |
| 231 | | | |
| Breast ca. BT 549 | 13.5 | Fetal Skeletal Muscle | 0.6 |
| Breast ca. T47D | 100.0 | Skeletal Muscle Pool | 3.5 |
| Breast ca. MDA-N | 9.7 | Spleen Pool | 4.5 |
| Breast Pool | 4.2 | Thymus Pool | 2.6 |
| Trachea | 2.6 | CNS cancer (glio/astro) | 12.5 |
| | | U87-MG | |
| Lung | 0.4 | CNS cancer (glio/astro) U- | 9.1 |
| | | 118-MG | |
| Fetal Lung | 3.7 | CNS cancer (neuro; met) | 11.3 |
| | | SK-N-AS | |
| Lung ca. NCI-N417 | 1.9 | CNS cancer (astro) SF-539 | 5.1 |
| Lung ca. LX-1 | 6.9 | CNS cancer (astro) SNB- | 13.5 |
| | | 75 | |
| Lung ca. NCI-H146 | 4.2 | CNS cancer (glio) SNB-19 | 13.3 |
| Lung ca. SHP-77 | 1.4 | CNS cancer (glio) SF-295 | 21.8 |
| Lung ca. A549 | 3.8 | Brain (Amygdala) Pool | 3.8 |
| Lung ca. NCI-H526 | 5.7 | Brain (cerebellum) | 4.7 |
| Lung ca. NCI-H23 | 4.7 | Brain (fetal) | 1.4 |
| Lung ca. NCI-H460 | 2.8 | Brain (Hippocampus) Pool | 4.7 |
| Lung ca. HOP-62 | 6.7 | Cerebral Cortex Pool | 6.4 |
| Lung ca. NCI-H522 | 4.5 | Brain (Substantia nigra) | 9.3 |
| | | Pool | |
| Liver | 2.0 | Brain (Thalamus) Pool | 6.0 |
| Fetal Liver | 2.4 | Brain (whole) | 2.8 |
| Liver ca. HepG2 | 4.5 | Spinal Cord Pool | 6.4 |
| Kidney Pool | 4.5 | Adrenal Gland | 3.1 |
| Fetal Kidney | 3.2 | Pituitary gland Pool | 4.3 |
| Renal ca. 786-0 | 6.7 | Salivary Gland | 4.6 |
| Renal ca. A498 | 5.4 | Thyroid (female) | 6.9 |
| Renal ca. ACHN | 3.6 | Pancreatic ca. CAPAN2 | 9.5 |
| Renal ca. UO-31 | 6.3 | Pancreas Pool | 6.0 |

TABLE LD
Panel 4.1D

| | Rel. Exp. (%) Ag4246, | | Rel. Exp. (%) Ag4246, |
|---|---|---|---|
| Tissue Name | Run 175165709 | Tissue Name | Run 175165709 |
| Secondary Th1 act | 9.5 | HUVEC IL-1beta | 17.1 |
| Secondary Th2 act | 28.9 | HUVEC IFN gamma | 18.6 |
| Secondary Tr1 act | 13.6 | HUVEC TNF alpha + IFN | 12.2 |
| | | gamma | |
| Secondary Th1 rest | 12.5 | HUVEC TNF alpha + IL4 | 11.2 |
| Secondary Th2 rest | 11.9 | HUVEC IL-11 | 23.0 |
| Secondary Tr1 rest | 21.9 | Lung Microvascular EC none | 40.1 |
| Primary Th1 act | 19.8 | Lung Microvascular EC | 40.6 |
| | | TNF alpha + IL-1beta | |
| Primary Th2 act | 34.4 | Microvascular Dermal EC | 26.2 |
| | | none | |
| Primary Tr1 act | 25.0 | Microsvascular Dermal EC | 12.9 |
| | | TNF alpha + IL-1beta | |
| Primary Th1 rest | 11.8 | Bronchial epithelium | 21.3 |
| | | TNF alpha + IL1beta | |
| Primary Th2 rest | 11.2 | Small airway epithelium none | 13.3 |
| Primary Tr1 rest | 11.5 | Small airway epithelium | 43.2 |
| | | TNF alpha + IL-1beta | |
| CD45RA CD4 | 9.4 | Coronery artery SMC rest | 25.0 |
| lymphocyte act | | | |
| CD45RO CD4 | 10.4 | Coronery artery SMC | 16.5 |
| lymphocyte act | | TNF alpha + IL-1beta | |
| CD8 lymphocyte act | 26.4 | Astrocytes rest | 20.3 |
| Secondary CD8 | 24.0 | Astrocytes TNF alpha + IL- | 15.3 |
| lymphocyte rest | | 1beta | |
| Secondary CD8 | 22.4 | KU-812 (Basophil) rest | 10.4 |
| lymphocyte act | | | |
| CD4 lymphocyte none | 13.6 | KU-812 (Basophil) | 8.4 |
| | | PMA/ionomycin | |
| 2ry Th1/Th2/Tr1_anti- | 28.9 | CCD1106 (Keratinocytes) | 28.1 |
| CD95 CH11 | | none | |
| LAK cells rest | 24.1 | CCD1106 (Keratinocytes) | 20.3 |
| | | TNF alpha + IL-1beta | |
| LAK cells IL-2 | 10.9 | Liver cirrhosis | 36.3 |
| LAK cells IL-2 + IL-12 | 14.5 | NCI-H292 none | 67.4 |
| LAK cells IL-2 + IFN | 17.4 | NCI-H292-IL4 | 66.9 |
| gamma | | | |
| LAK cells IL-2 + IL-18 | 22.1 | NCI-H292 IL-9 | 74.7 |
| LAK cells | 1.4 | NCI-H292 IL-13 | 38.4 |
| PMA/ionomycin | | | |
| NK Cells IL-2 rest | 19.1 | NCI-H292 IFN gamma | 60.7 |
| Two Way MLR 3 day | 33.4 | HPAEC none | 15.9 |
| Two Way MLR 5 day | 14.1 | HPAEC TNF alpha + IL-1beta | 26.6 |
| Two Way MLR 7 day | 22.1 | Lung fibroblast none | 45.7 |
| PBMC rest | 18.0 | Lung fibroblast TNF alpha + | 55.1 |
| | | IL-1beta | |
| PBMC PWM | 23.8 | Lung fibroblast IL-4 | 54.3 |
| PBMC PHA-L | 21.8 | Lung fibroblast IL-9 | 77.9 |
| Ramos (B cell) none | 67.8 | Lung fibroblast IL-13 | 32.1 |
| Ramos (B cell) ionomycin | 64.6 | Lung fibroblast IFN gamma | 32.1 |
| B lymphocytes PWM | 8.5 | Dermal fibroblast CCD1070 | 33.0 |
| | | rest | |
| B lymphocytes CD40L | 17.0 | Dermal fibroblast CCD1070 | 15.3 |
| and IL-4 | | TNF alpha | |
| EOL-1 dbcAMP | 27.4 | Dermal fibroblast CCD1070 | 12.2 |
| | | IL-1beta | |
| EOL-1 dbcAMP | 0.0 | Dermal fibroblast IFN gamma | 24.8 |
| PMA/ionomycin | | | |
| Dendritic cells none | 50.0 | Dermal fibroblast IL-4 | 27.4 |
| Dendritic cells LPS | 36.9 | Dermal fibroblast rest | 38.2 |
| Dendritic cells anti-CD40 | 40.1 | Neutrophils TNFa + LPS | 0.0 |
| Monocytes rest | 37.1 | Neutrophils rest | 9.2 |
| Monocytes LPS | 4.7 | Colon | 23.7 |
| Macrophages rest | 37.9 | Lung | 16.2 |
| Macrophages LPS | 27.2 | Thymus | 39.5 |
| HUVEC none | 20.2 | Kidney | 100.0 |
| HUVEC starved | 19.2 | | |

TABLE LE
general oncology screening panel_v_2.4

| | Rel. Exp. (%) Ag4246, Run | | Rel. Exp. (%) Ag4246, Run |
|---|---|---|---|
| Tissue Name | 268664320 | Tissue Name | 268664320 |
| Colon cancer 1 | 63.7 | Bladder cancer NAT 2 | 2.4 |
| Colon cancer NAT 1 | 33.4 | Bladder cancer NAT 3 | 1.7 |
| Colon cancer 2 | 17.8 | Bladder cancer NAT 4 | 16.3 |
| Colon cancer NAT 2 | 23.3 | Adenocarcinoma of the | 23.8 |
| | | prostate 1 | |
| Colon cancer 3 | 82.4 | Adenocarcinoma of the | 11.2 |
| | | prostate 2 | |
| Colon cancer NAT 3 | 39.8 | Adenocarcinoma of the | 38.2 |
| | | prostate 3 | |
| Colon malignant | 50.3 | Adenocarcinoma of the | 25.3 |
| cancer 4 | | prostate 4 | |
| Colon normal adjacent | 10.2 | Prostate cancer NAT 5 | 15.8 |
| tissue 4 | | | |
| Lung cancer 1 | 26.1 | Adenocarcinoma of the | 12.9 |
| | | prostate 6 | |
| Lung NAT 1 | 1.9 | Adenocarcinoma of the | 16.4 |
| | | prostate 7 | |
| Lung cancer 2 | 74.7 | Adenocarcinoma of the | 2.9 |
| | | prostate 8 | |
| Lung NAT 2 | 5.1 | Adenocarcinoma of the | 33.7 |
| | | prostate 9 | |
| Squamous cell | 25.7 | Prostate cancer NAT 10 | 14.9 |
| carcinoma 3 | | | |
| Lung NAT 3 | 1.9 | Kidney cancer 1 | 49.7 |
| metastatic melanoma 1 | 22.5 | KidneyNAT 1 | 23.3 |
| Melanoma 2 | 6.6 | Kidney cancer 2 | 87.1 |
| Melanoma 3 | 2.8 | Kidney NAT 2 | 90.1 |
| metastatic melanoma 4 | 45.7 | Kidney cancer 3 | 90.8 |
| metastatic melanoma 5 | 43.8 | Kidney NAT 3 | 20.3 |
| Bladder cancer 1 | 0.4 | Kidney cancer 4 | 100.0 |
| Bladder cancer NAT 1 | 0.0 | Kidney NAT 4 | 63.7 |
| Bladder cancer 2 | 11.6 | | |

CNS_neurodegeneration_v1.0 Summary: Ag4246 This panel confirms the expression of the CG102878-01 gene at low levels in the brain in an independent group of individuals. This gene is found to be down-regulated in the temporal cortex of Alzheimer's disease patients. Therefore, up-regulation of this gene or its protein product, or treatment with specific agonists for this receptor may be of use in reversing the dementia/memory loss associated with this disease and neuronal death.

General_screening_panel_v1.4 Summary: Ag4246 Highest expression of the CG102878-01 gene is detected in breast cancer T47D cell line (CT=26.2). Significant expression of this gene is also seen in clusters of cancer cell lines derived from melanoma, pancreatic, renal, gastric, colon, lung, breast, ovarian and brain cancers. Thus, expression of this gene could be used as a marker to detect the presence of these cancers. Furthermore, therapeutic modulation of the expression or function of this gene may be effective in the treatment of gastric, colon, lung, breast, ovarian and brain cancer.

Among tissues with metabolic or endocrine function, this gene is expressed at high to moderate levels in pancreas, adipose, adrenal gland, thyroid, pituitary gland, skeletal muscle, heart, liver and the gastrointestinal tract. Therefore, therapeutic modulation of the activity of this gene may prove useful in the treatment of endocrine/metabolically related diseases, such as obesity and diabetes.

In addition, this gene is expressed at moderate levels in all regions of the central nervous system examined, including amygdala, hippocampus, substantia nigra, thalamus, cerebellum, cerebral cortex, and spinal cord. Therefore, this gene may play a role in central nervous system disorders such as Alzheimer's disease, Parkinson's disease, epilepsy, multiple sclerosis, schizophrenia and depression.

Panel 4.1D Summary: Ag4246 Highest expression of the CG102878-01 gene is detected in kidney (CT=31.9). This gene is expressed at moderate to low levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. This pattern is in agreement with the expression profile in General_screening_panel_v1.4 and also suggests a role for the gene product in cell survival and proliferation. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

general oncology screening panel_v_{—}2.4 Summary: Ag4246 Highest expression of the CG02878-01 gene is detected in kidney cancer sample (CT=31). Moderate to low levels of expression of this gene is seen in normal and cancer samples derived from kidney, colon, lung, and prostate. Significant expression of this gene is also seen in metastatic melanoma. Interestingly, expression of this gene is higher in lung cancer samples as compared to adjacent control samples. Therefore, expression of this gene may be used as diagnostic marker for lung cancer and metastic melanoma. Furtherermore, therapeutic modulation of this gene product may be beneficial in the treatment of melanoma, colon, lung, prostate and some cases of kidney cancer.

M. CG103459-01: Novel Peptide/Histidine Transporter

Expression of gene CG103459-01 was assessed using the primer-probe set Ag4262, described in Table MA. Results of the RTQ-PCR runs are shown in Tables MB, MC and MD.

TABLE MA

Probe Name Ag4262

| | | | Start | SEQ ID |
|---|---|---|---|---|
| Primers | Sequences | Length | Position | No |
| Forward | 5′-cagagtaatggtgaaggcattg-3′ | 22 | 845 | 186 |
| Probe | TET-′-tcagcaatcttctaaacaagtctgtttga-3′-TAMRA | 30 | 874 | 187 |
| Reverse | 5′-cccaccatgagacatcttacat-3′ | 22 | 907 | 188 |

TABLE MB
CNS_neurodegeneration_v1.0

| | Rel. Exp. (%) Ag4262, Run | | Rel. Exp. (%) Ag4262, Run |
|---|---|---|---|
| Tissue Name | 224076196 | Tissue Name | 224076196 |
| AD 1 Hippo | 6.8 | Control (Path) 3 | 3.5 |
| | | Temporal Ctx | |
| AD 2 Hippo | 18.8 | Control (Path) 4 | 15.3 |
| | | Temporal Ctx | |
| AD 3 Hippo | 6.7 | AD 1 Occipital Ctx | 11.0 |
| AD 4 Hippo | 10.2 | AD 2 Occipital Ctx | 0.0 |
| | | (Missing) | |
| AD 5 Hippo | 32.8 | AD 3 Occipital Ctx | 4.9 |
| AD 6 Hippo | 100.0 | AD 4 Occipital Ctx | 12.2 |
| Control 2 Hippo | 17.6 | AD 5 Occipital Ctx | 20.0 |
| Control 4 Hippo | 42.0 | AD 6 Occipital Ctx | 32.3 |
| Control (Path) 3 Hippo | 20.6 | Control 1 Occipital Ctx | 3.6 |
| AD 1 Temporal Ctx | 10.7 | Control 2 Occipital Ctx | 29.1 |
| AD 2 Temporal Ctx | 19.5 | Control 3 Occipital Ctx | 8.2 |
| AD 3 Temporal Ctx | 4.0 | Control 4 Occipital Ctx | 7.4 |
| AD 4 Temporal Ctx | 14.4 | Control (Path) 1 | 27.4 |
| | | Occipital Ctx | |
| AD 5 Inf Temporal Ctx | 42.9 | Control (Path) 2 | 6.0 |
| | | Occipital Ctx | |
| AD 5 Sup Temporal | 68.8 | Control (Path) 3 | 4.1 |
| Ctx | | Occipital Ctx | |
| AD 6 Inf Temporal Ctx | 72.7 | Control (Path) 4 | 9.9 |
| | | Occipital Ctx | |
| AD 6 Sup Temporal | 68.8 | Control 1 Parietal Ctx | 4.7 |
| Ctx | | | |
| Control 1 Temporal | 7.7 | Control 2 Parietal Ctx | 33.2 |
| Ctx | | | |
| Control 2 Temporal | 16.8 | Control 3 Parietal Ctx | 9.0 |
| Ctx | | | |
| Control 3 Temporal | 9.4 | Control (Path) 1 | 27.0 |
| Ctx | | Parietal Ctx | |
| Control 3 Temporal | 9.5 | Control (Path) 2 | 12.6 |
| Ctx | | Parietal Ctx | |
| Control (Path) 1 | 25.9 | Control (Path) 3 | 4.3 |
| Temporal Ctx | | Parietal Ctx | |
| Control (Path) 2 | 15.6 | Control (Path) 4 | 14.8 |
| Temporal Ctx | | Parietal Ctx | |

TABLE MC
General_screening_panel_v1.4

| | Rel. Exp. (%) Ag4262, | | Rel. Exp. (%) Ag4262, |
|---|---|---|---|
| Tissue Name | Run 222046622 | Tissue Name | Run 222046622 |
| Adipose | 8.0 | Renal ca. TK-10 | 34.2 |
| Melanoma* | 35.4 | Bladder | 15.8 |
| Hs688(A).T | | | |
| Melanoma* | 41.2 | Gastric ca. (liver met.) | 20.6 |
| Hs688(B).T | | NCI-N87 | |
| Melanoma* M14 | 97.9 | Gastric ca. KATO III | 36.3 |
| Melanoma* LOXIMVI | 37.9 | Colon ca. SW-948 | 13.4 |
| Melanoma* SK-MEL-5 | 40.9 | Colon ca. SW480 | 40.9 |
| Squamous cell | 10.2 | Colon ca.* (SW480 met) | 19.9 |
| carcinoma SCC-4 | | SW620 | |
| Testis Pool | 16.6 | Colon ca. HT29 | 15.9 |
| Prostate ca.* (bone met) | 44.8 | Colon ca. HCT-116 | 47.6 |
| PC-3 | | | |
| Prostate Pool | 5.3 | Colon ca. CaCo-2 | 20.6 |
| Placenta | 8.2 | Colon cancer tissue | 36.3 |
| Uterus Pool | 7.5 | Colon ca. SW1116 | 2.8 |
| Ovarian ca. OVCAR-3 | 26.2 | Colon ca. Colo-205 | 9.9 |
| Ovarian ca. SK-OV-3 | 55.9 | Colon ca. SW-48 | 8.8 |
| Ovarian ca. OVCAR-4 | 6.5 | Colon Pool | 18.8 |
| Ovarian ca. OVCAR-5 | 34.9 | Small Intestine Pool | 12.2 |
| Ovarian ca. IGROV-1 | 25.3 | Stomach Pool | 9.4 |
| Ovarian ca. OVCAR-8 | 20.0 | Bone Marrow Pool | 8.4 |
| Ovary | 9.0 | Fetal Heart | 5.0 |
| Breast ca. MCF-7 | 27.0 | Heart Pool | 7.5 |
| Breast ca. MDA-MB- | 46.3 | Lymph Node Pool | 26.4 |
| 231 | | | |
| Breast ca. BT 549 | 63.7 | Fetal Skeletal Muscle | 7.5 |
| Breast ca. T47D | 78.5 | Skeletal Muscle Pool | 17.0 |
| Breast ca. MDA-N | 23.3 | Spleen Pool | 9.6 |
| Breast Pool | 19.2 | Thymus Pool | 14.7 |
| Trachea | 12.9 | CNS cancer (glio/astro) | 57.8 |
| | | U87-MG | |
| Lung | 2.0 | CNS cancer (glio/astro) U- | 65.5 |
| | | 118-MG | |
| Fetal Lung | 19.8 | CNS cancer (neuro; met) | 33.4 |
| | | SK-N-AS | |
| Lung ca. NCI-N417 | 3.6 | CNS cancer (astro) SF-539 | 30.6 |
| Lung ca. LX-1 | 25.9 | CNS cancer (astro) SNB- | 100.0 |
| | | 75 | |
| Lung ca. NCI-H146 | 7.6 | CNS cancer (glio) SNB-19 | 23.2 |
| Lung ca. SHP-77 | 17.7 | CNS cancer (glio) SF-295 | 47.3 |
| Lung ca. A549 | 44.1 | Brain (Amygdala) Pool | 6.2 |
| Lung ca. NCI-H526 | 7.4 | Brain (cerebellum) | 12.1 |
| Lung ca. NCI-H23 | 33.7 | Brain (fetal) | 6.7 |
| Lung ca. NCI-H460 | 22.7 | Brain (Hippocampus) Pool | 8.8 |
| Lung ca. HOP-62 | 25.5 | Cerebral Cortex Pool | 7.5 |
| Lung ca. NCI-H522 | 31.4 | Brain (Substantia nigra) | 7.6 |
| | | Pool | |
| Liver | 4.4 | Brain (Thalamus) Pool | 9.8 |
| Fetal Liver | 17.8 | Brain (whole) | 9.4 |
| Liver ca. HepG2 | 19.3 | Spinal Cord Pool | 13.2 |
| Kidney Pool | 26.2 | Adrenal Gland | 9.7 |
| Fetal Kidney | 9.5 | Pituitary gland Pool | 3.7 |
| Renal ca. 786-0 | 69.3 | Salivary Gland | 6.1 |
| Renal ca. A498 | 20.6 | Thyroid (female) | 8.2 |
| Renal ca. ACHN | 38.4 | Pancreatic ca. CAPAN2 | 31.4 |
| Renal ca. UO-31 | 94.0 | Pancreas Pool | 45.7 |

TABLE MD
Panel 4.1D

| | Rel. Exp. (%) Ag4262, | | Rel. Exp. (%) Ag4262, |
|---|---|---|---|
| Tissue Name | Run 176243568 | Tissue Name | Run 176243568 |
| Secondary Th1 act | 37.1 | HUVEC IL-1beta | 28.9 |
| Secondary Th2 act | 30.4 | HUVEC IFN gamma | 18.0 |
| Secondary Tr1 act | 14.1 | HUVEC TNF alpha + IFN | 34.9 |
| | | gamma | |
| Secondary Th1 rest | 9.0 | HUVEC TNF alpha + IL4 | 28.5 |
| Secondary Th2 rest | 6.4 | HUVEC IL-11 | 8.4 |
| Secondary Tr1 rest | 7.4 | Lung Microvascular EC none | 27.7 |
| Primary Th1 act | 14.7 | Lung Microvascular EC | 28.3 |
| | | TNF alpha + IL-1beta | |
| Primary Th2 act | 17.4 | Microvascular Dermal EC | 16.0 |
| | | none | |
| Primary Tr1 act | 22.5 | Microsvasular Dermal EC | 18.9 |
| | | TNF alpha + IL-1beta | |
| Primary Th1 rest | 5.6 | Bronchial epithelium | 39.8 |
| | | TNF alpha + IL1beta | |
| Primary Th2 rest | 2.5 | Small airway epithelium none | 14.6 |
| Primary Tr1 rest | 13.5 | Small airway epithelium | 47.0 |
| | | TNF alpha + IL-1beta | |
| CD45RA CD4 | 40.1 | Coronery artery SMC rest | 20.9 |
| lymphocyte act | | | |
| CD45RO CD4 | 33.7 | Coronery artery SMC | 25.9 |
| lymphocyte act | | TNF alpha + IL-1beta | |
| CD8 lymphocyte act | 31.6 | Astrocytes rest | 22.5 |
| Secondary CD8 | 20.4 | Astrocytes TNF alpha + IL- | 22.2 |
| lymphocyte rest | | 1beta | |
| Secondary CD8 | 9.7 | KU-812 (Basophil) rest | 14.0 |
| lymphocyte act | | | |
| CD4 lymphocyte none | 7.0 | KU-812 (Basophil) | 16.7 |
| | | PMA/ionomycin | |
| 2ry Th1/Th2/Tr1_anti- | 12.3 | CCD1106 (Keratinocytes) | 44.8 |
| CD95 CH11 | | none | |
| LAK cells rest | 46.0 | CCD1106 (Keratinocytes) | 27.4 |
| | | TNF alpha + IL-1beta | |
| LAK cells IL-2 | 28.9 | Liver cirrhosis | 7.5 |
| LAK cells IL-2 + IL-12 | 18.2 | NCI-H292 none | 18.0 |
| LAK cells IL-2 + IFN | 21.8 | NCI-H292 IL-4 | 24.0 |
| gamma | | | |
| LAK cells IL-2 + IL-18 | 34.2 | NCI-H292 IL-9 | 33.2 |
| LAK cells | 26.8 | NCI-H292 IL-13 | 27.5 |
| PMA/ionomycin | | | |
| NK Cells IL-2 rest | 71.7 | NCI-H292 IFN gamma | 20.6 |
| Two Way MLR 3 day | 41.5 | HPAEC none | 13.0 |
| Two Way MLR 5 day | 41.5 | HPAEC TNF alpha + IL-1beta | 41.2 |
| Two Way MLR 7 day | 25.9 | Lung fibroblast none | 27.7 |
| PBMC rest | 15.3 | Lung fibroblast TNF alpha + | 29.3 |
| | | IL-1beta | |
| PBMC PWM | 27.0 | Lung fibroblast IL-4 | 24.7 |
| PBMC PHA-L | 43.8 | Lung fibroblast IL-9 | 34.9 |
| Ramos (B cell) none | 32.3 | Lung fibroblast IL-13 | 24.5 |
| Ramos (B cell) ionomycin | 51.1 | Lung fibroblast IFN gamma | 52.1 |
| B lymphocytes PWM | 28.3 | Dermal fibroblast CCD1070 | 31.6 |
| | | rest | |
| B lymphocytes CD40L | 66.4 | Dermal fibroblast CCD1070 | 45.1 |
| and IL-4 | | TNF alpha | |
| EOL-1 dbcAMP | 17.3 | Dermal fibroblast CCD1070 | 28.1 |
| | | IL-1beta | |
| EOL-1 dbcAMP | 18.2 | Dermal fibroblast IFN gamma | 28.1 |
| PMA/ionomycin | | | |
| Dendritic cells none | 44.8 | Dermal fibroblast IL-4 | 38.7 |
| Dendritic cells LPS | 36.1 | Dermal Fibroblasts rest | 22.5 |
| Dendritic cells anti-CD40 | 42.9 | Neutrophils TNFa + LPS | 56.6 |
| Monocytes rest | 49.0 | Neutrophils rest | 100.0 |
| Monocytes LPS | 47.6 | Colon | 9.4 |
| Macrophages rest | 52.9 | Lung | 16.4 |
| Macrophages LPS | 21.9 | Thymus | 23.8 |
| HUVEC none | 9.9 | Kidney | 15.2 |
| HUVEC starved | 21.2 | | |

CNS_neurodegeneration_v1.0 Summary: Ag4262 This panel confirms the expression of the CG103459-01 gene at low levels in the brains of an independent group of individuals.

However, no differential expression of this gene was detected between Alzheimer's diseased postmortem brains and those of non-demented controls in this experiment. Please see Panel 1.4 for a discussion of the potential use of this gene in treatment of central nervous system disorders.

General_screening_panel_v1.4 Summary: Ag4262 Highest expression of the CG103459-01 gene is detected in CNS cancer (astro) SNB-75 cell line (28.7). High to moderate levels of expression of this gene is seen in cluster of cancer cell lines derived from pancreatic, gastric, colon, renal, lung, breast, ovarian, prostate, squamous cell carcinoma and brain cancers. Thus, expression of this gene could be used as a marker to detect the presence of these cancers. Furthermore, therapeutic modulation of the expression or function of this gene may be effective in the treatment of these cancers.

Among tissues with metabolic or endocrine function, this gene is expressed at high to moderate levels in pancreas, adipose, adrenal gland, thyroid, pituitary gland, skeletal muscle, heart, liver and the gastrointestinal tract. Therefore, therapeutic modulation of the activity of this gene may prove useful in the treatment of endocrine/metabolically related diseases, such as obesity and diabetes.

In addition, this gene is expressed at moderate levels in all regions of the central nervous system examined, including amygdala, hippocampus, substantia nigra, thalamus, cerebellum, cerebral cortex, and spinal cord. Therefore, this gene may play a role in central nervous system disorders such as Alzheimer's disease, Parkinson's disease, epilepsy, multiple sclerosis, schizophrenia and depression.

Panel 4.1D Summary: Ag4262 Highest expression of the CG103459-01 gene is detected in resting neutrophils (CT=29.3). This gene is expressed at high to moderate levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. This pattern is in agreement with the expression profile in General_screening_panel_v1.4 and also suggests a role for the gene product in cell survival and proliferation. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

N. CG104210-01: Type III Membrane Protein

Expression of gene CG104210-01 was assessed using the primer-probe set Ag4270, described in Table NA. Results of the RTQ-PCR runs are shown in Tables NB, NC, ND and NE.

TABLE NA

Probe Name Ag4270

| | | | Start | SEQ ID |
|---|---|---|---|---|
| Primers | Sequences | Length | Position | No |
| Forward | 5′-acctgcagctgagaaaatcc-3′ | 20 | 1250 | 189 |
| Probe | TET-5′-ctcaaccacctcgtctcctccatcg-3′-TAMRA | 25 | 2270 | 190 |
| Reverse | 5′-aggcatacaacccactgtca-3′ | 20 | 1316 | 191 |

TABLE NB
CNS_neurodegeneration_v1.0

| | Rel. Exp. (%) Ag4270, Run | | Rel. Exp. (%) Ag4270, Run |
|---|---|---|---|
| Tissue Name | 224075728 | Tissue Name | 224075728 |
| AD 1 Hippo | 69.7 | Control (Path) 3 | 0.0 |
| | | Temporal Ctx | |
| AD 2 Hippo | 36.9 | Control (Path) 4 | 0.0 |
| | | Temporal Ctx | |
| AD 3 Hippo | 0.0 | AD 1 Occipital Ctx | 0.0 |
| AD 4 Hippo | 0.0 | AD 2 Occipital Ctx | 0.0 |
| | | (Missing) | |
| AD 5 Hippo | 0.0 | AD 3 Occipital Ctx | 0.0 |
| AD 6 Hippo | 0.0 | AD 4 Occipital Ctx | 27.7 |
| Control 2 Hippo | 20.7 | AD 5 Occipital Ctx | 0.0 |
| Control 4 Hippo | 100.0 | AD 6 Occipital Ctx | 22.2 |
| Control (Path) 3 Hippo | 37.9 | Control 1 Occipital Ctx | 17.3 |
| AD 1 Temporal Ctx | 0.0 | Control 2 Occipital Ctx | 21.6 |
| AD 2 Temporal Ctx | 0.0 | Control 3 Occipital Ctx | 36.9 |
| AD 3 Temporal Ctx | 0.0 | Control 4 Occipital Ctx | 0.0 |
| AD 4 Temporal Ctx | 35.6 | Control (Path) 1 | 34.4 |
| | | Occipital Ctx | |
| AD 5 Inf Temporal Ctx | 0.0 | Control (Path) 2 | 0.0 |
| | | Occipital Ctx | |
| AD 5 Sup Temporal | 30.6 | Control (Path) 3 | 0.0 |
| Ctx | | Occipital Ctx | |
| AD 6 Inf Temporal Ctx | 37.1 | Control (Path) 4 | 0.0 |
| | | Occipital Ctx | |
| AD 6 Sup Temporal | 73.7 | Control 1 Parietal Ctx | 0.0 |
| Ctx | | | |
| Control 1 Temporal | 11.9 | Control 2 Parietal Ctx | 18.2 |
| Ctx | | | |
| Control 2 Temporal | 0.0 | Control 3 Parietal Ctx | 0.0 |
| Ctx | | | |
| Control 3 Temporal | 17.1 | Control (Path) 1 | 11.3 |
| Ctx | | Parietal Ctx | |
| Control 3 Temporal | 0.0 | Control (Path) 2 | 14.5 |
| Ctx | | Parietal Ctx | |
| Control (Path) 1 | 41.5 | Control (Path) 3 | 0.0 |
| Temporal Ctx | | Parietal Ctx | |
| Control (Path) 2 | 0.0 | Control (Path) 4 | 49.3 |
| Temporal Ctx | | Parietal Ctx | |

TABLE NC
HASS Panel v1.0

| Tissue | Rel. Exp. (%) Ag4270, Run | | Rel. Exp. (%) Ag4270, Run |
|---|---|---|---|
| Name | 268623848 | Tissue Name | 268623848 |
| MCF-7 C1 | 13.7 | U87-MG F1 (B) | 0.0 |
| MCF-7 C2 | 18.4 | U87-MG F2 | 0.0 |
| MCF-7 C3 | 8.7 | U87-MG F3 | 2.4 |
| MCF-7 C4 | 22.5 | U87-MG F4 | 0.0 |
| MCF-7 C5 | 9.9 | U87-MG F5 | 0.0 |
| MCF-7 C6 | 12.6 | U87-MG F6 | 0.0 |
| MCF-7 C7 | 29.9 | U87-MG F7 | 0.0 |
| MCF-7 C9 | 8.7 | U87-MG F8 | 0.0 |
| MCF-7 | 29.9 | U87-MG F9 | 0.0 |
| C10 | | | |
| MCF-7 | 2.7 | U87-MG F10 | 0.0 |
| C11 | | | |
| MCF-7 | 12.7 | U87-MG F11 | 0.0 |
| C12 | | | |
| MCF-7 | 40.3 | U87-MG F12 | 0.0 |
| C13 | | | |
| MCF-7 | 10.1 | U87-MG F13 | 0.0 |
| C15 | | | |
| MCF-7 | 10.1 | U87-MG F14 | 1.3 |
| C16 | | | |
| MCF-7 | 15.0 | U87-MG F15 | 0.0 |
| C17 | | | |
| T24 D1 | 0.0 | U87-MG F16 | 0.0 |
| T24 D2 | 0.0 | U87-MG F17 | 0.0 |
| T24 D3 | 0.0 | LnCAP A1 | 60.3 |
| T24 D4 | 0.0 | LnCAP A2 | 40.1 |
| T24 D5 | 0.0 | LnCAP A3 | 74.2 |
| T24 D6 | 0.0 | LnCAP A4 | 26.1 |
| T24 D7 | 0.0 | LnCAP A5 | 33.0 |
| T24 D9 | 0.0 | LnCAP A6 | 23.0 |
| T24 D10 | 0.0 | LnCAP A7 | 68.3 |
| T24 D11 | 0.0 | LnCAP A8 | 69.7 |
| T24 D12 | 0.0 | LnCAP A9 | 38.4 |
| T24 D13 | 0.0 | LnCAP A10 | 62.0 |
| T24 D15 | 0.0 | LnCAP A11 | 100.0 |
| T24 D16 | 0.0 | LnCAP A12 | 13.2 |
| T24 D17 | 0.0 | LnCAP A13 | 51.4 |
| CAPaN B1 | 39.0 | LnCAP A14 | 30.1 |
| CAPaN B2 | 16.8 | LnCAP A15 | 22.5 |
| CAPaN B3 | 17.7 | LnCAP A16 | 48.0 |
| CAPaN B4 | 16.8 | LnCAP A17 | 70.2 |
| CAPaN B5 | 26.6 | Primary Astrocytes | 0.0 |
| CAPaN B6 | 5.7 | Primary Renal Proximal Tubule | 0.0 |
| | | Epithelial cell A2 | |
| CAPaN B7 | 24.3 | Primary melanocytes A5 | 0.0 |
| CAPaN B8 | 27.2 | 126443-341 medullo | 4.2 |
| CAPaN B9 | 18.3 | 126444-487 medullo | 0.0 |
| CAPaN | 67.8 | 126445-425 medullo | 0.0 |
| B10 | | | |
| CAPaN | 81.8 | 126446-690 medullo | 1.3 |
| B11 | | | |
| CAPaN | 23.0 | 126447-54 adult glioma | 0.0 |
| B12 | | | |
| CAPaN | 71.2 | 126448-245 adult glioma | 0.0 |
| B13 | | | |
| CAPaN | 27.9 | 126449-317 adult glioma | 0.0 |
| B14 | | | |
| CAPaN | 14.8 | 126450-212 glioma | 0.0 |
| B15 | | | |
| CAPaN | 17.4 | 126451-456 glioma | 0.0 |
| B16 | | | |
| CAPaN | 48.3 | | |
| B17 | | | |

TABLE ND
Panel 4.1D

| | Rel. Exp. (%) Ag4270, | | Rel. Exp. (%) Ag4270, |
|---|---|---|---|
| Tissue Name | Run 181080817 | Tissue Name | Run 181080817 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.4 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.5 |
| Secondary Tr1 rest | 0.0 | Lung Microvascular EC none | 0.0 |
| Primary Th1 act | 0.0 | Lung Microvascular EC | 0.0 |
| | | TNF alpha + IL-1beta | |
| Primary Th2 act | 0.0 | Microvascular Dermal EC | 0.0 |
| | | none | |
| Primary Tr1 act | 0.0 | Microsvascular Dermal EC | 0.0 |
| | | TNF alpha + IL-1beta | |
| Primary Th1 rest | 0.0 | Bronchial epithelium | 1.3 |
| | | TNF alpha + IL1beta | |
| Primary Th2 rest | 0.0 | Small airway epithelium none | 2.0 |
| Primary Tr1 rest | 0.0 | Small airway epithelium | 6.2 |
| | | TNF alpha + IL-1beta | |
| CD45RA CD4 | 100.0 | Coronery artery SMC rest | 0.0 |
| lymphocyte act | | | |
| CD45RO CD4 | 0.0 | Coronery artery SMC | 0.0 |
| lymphocyte act | | TNF alpha + IL-1beta | |
| CD8 lymphocyte act | 0.0 | Astrocytes rest | 0.0 |
| Secondary CD8 | 0.0 | Astrocytes TNF alpha + IL- | 0.0 |
| lymphocyte rest | | 1beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CCD1106 (Keratinocytes) | 1.5 |
| CD95 CH11 | | none | |
| LAK cells rest | 0.0 | CCD1106 (Keratinocytes) | 0.9 |
| | | TNF alpha + IL-1beta | |
| LAK cells IL-2 | 0.4 | Liver cirrhosis | 0.0 |
| LAK cells IL-2 + IL-12 | 0.0 | NCI-H292 none | 0.8 |
| LAK cells IL-2 + IFN | 0.0 | NCI-H292 IL-4 | 0.5 |
| gamma | | | |
| LAK cells IL-2 + IL-18 | 0.0 | NCI-H292 IL-9 | 1.6 |
| LAK cells | 5.8 | NCI-H292 IL-13 | 0.5 |
| PMA/ionomycin | | | |
| NK Cells IL-2 rest | 0.0 | NCI-H292 IFN gamma | 0.3 |
| Two Way MLR 3 day | 0.0 | HPAEC none | 0.0 |
| Two Way MLR 5 day | 0.5 | HPAEC TNF alpha + IL-1beta | 0.0 |
| Two Way MLR 7 day | 0.0 | Lung fibroblast none | 0.0 |
| PBMC rest | 0.0 | Lung fibroblast TNF alpha + | 0.0 |
| | | IL-1beta | |
| PBMC PWM | 0.0 | Lung fibroblast IL-4 | 0.0 |
| PBMC PHA-L | 0.0 | Lung fibroblast IL-9 | 0.0 |
| Ramos (B cell) none | 0.0 | Lung fibroblast IL-13 | 0.0 |
| Ramos (B cell) ionomycin | 0.0 | Lung fibroblast IFN gamma | 0.0 |
| B lymphocytes PWM | 0.0 | Dermal fibroblast CCD1070 | 0.0 |
| | | rest | |
| B lymphocytes CD40L | 0.0 | Dermal fibroblast CCD1070 | 0.0 |
| and IL-4 | | TNF alpha | |
| EOL-1 dbcAMP | 0.0 | Dermal fibroblast CCD1070 | 0.0 |
| | | IL-1beta | |
| EOL-1 dbcAMP | 0.0 | Dermal fibroblast IFN gamma | 0.0 |
| PMA/ionomycin | | | |
| Dendritic cells none | 0.0 | Dermal fibroblast IL-4 | 0.0 |
| Dendritic cells LPS | 0.0 | Dermal Fibroblast rest | 0.7 |
| Dendritic cells anti-CD40 | 0.0 | Neutrophils TNFa + LPS | 0.5 |
| Monocytes rest | 0.0 | Neutrophils rest | 0.5 |
| Monocytes LPS | 1.2 | Colon | 0.0 |
| Macrophages rest | 0.0 | Lung | 1.6 |
| Macrophages LPS | 0.0 | Thymus | 4.1 |
| HUVEC none | 0.0 | Kidney | 30.1 |
| HUVEC starved | 0.0 | | |

TABLE NE
general oncology screening panel_v_2.4

| | Rel. Exp. (%) Ag4270, Run | | Rel. Exp. (%) Ag4270, Run |
|---|---|---|---|
| Tissue Name | 260280401 | Tissue Name | 260280401 |
| Colon cancer 1 | 3.8 | Bladder cancer NAT 2 | 0.0 |
| Colon cancer NAT 1 | 0.0 | Bladder cancer NAT 3 | 0.0 |
| Colon cancer 2 | 10.4 | Bladder cancer NAT 4 | 0.0 |
| Colon cancer NAT 2 | 0.0 | Adenocarcinoma of the | 4.3 |
| | | prostate 1 | |
| Colon cancer 3 | 3.3 | Adenocarcinoma of the | 11.1 |
| | | prostate 2 | |
| Colon cancer NAT 3 | 0.0 | Adenocarcinoma of the | 36.3 |
| | | prostate 3 | |
| Colon malignant | 2.0 | Adenocarcinoma of the | 9.9 |
| cancer 4 | | prostate 4 | |
| Colon normal adjacent | 0.0 | Prostate cancer NAT 5 | 73.7 |
| tissue 4 | | | |
| Lung cancer 1 | 82.9 | Adenocarcinoma of the | 12.1 |
| | | prostate 6 | |
| Lung NAT 1 | 11.5 | Adenocarcinoma of the | 9.9 |
| | | prostate 7 | |
| Lung cancer 2 | 9.3 | Adenocarcinoma of the | 4.0 |
| | | prostate 8 | |
| Lung NAT 2 | 3.3 | Adenocarcinoma of the | 18.3 |
| | | prostate 9 | |
| Squamous cell | 33.7 | Prostate cancer NAT 10 | 1.8 |
| carcinoma 3 | | | |
| Lung NAT 3 | 15.0 | Kidney cancer 1 | 0.0 |
| metastatic melanoma 1 | 100.0 | KidneyNAT 1 | 1.9 |
| Melanoma 2 | 81.8 | Kidney cancer 2 | 2.1 |
| Melanoma 3 | 81.8 | Kidney NAT 2 | 2.1 |
| metastatic melanoma 4 | 0.0 | Kidney cancer 3 | 0.0 |
| metastatic melanoma 5 | 2.2 | Kidney NAT 3 | 0.0 |
| Bladder cancer 1 | 0.0 | Kidney cancer 4 | 0.0 |
| Bladder cancer NAT 1 | 0.0 | Kidney NAT 4 | 0.0 |
| Bladder cancer 2 | 0.0 | | |

CNS_neurodegeneration_v1.0 Summary: Ag4270 This panel confirms the expression of the CG104210-01 gene at low levels in the brains of an independent group of individuals. However, no differential expression of this gene was detected between Alzheimer's diseased postmortem brains and those of non-demented controls in this experiment. Low expression of this gene in brain suggests that this gene may play role in neurological disorders such as Parkinson's disease, epilepsy, multiple sclerosis, schizophrenia and depression. Therefore, therapeutic modulation of this gene may be useful in the treatment of these neurological disorders.

HASS Panel v1.0 Summary: Ag4270 Highest expression of the CG104210-01 gene is detected in LnCAP (A11) cell line sample (CT=31.6) that are exposed to an acidic environment. CaPAN cells also show a modest increase in gene expression when exposed to an acidic environment (A10, A11 compared to A4, A5 resp.)

This suggests a possible induction of this gene in acidotic regions of prostate and pancreatic cancer.

Panel 4.1D Summary: Ag4270 Highest expression of the CG104210-01 gene is detected in activated CD45RA CD4 lymphocyte (CT=29), which represent activated naive T cells. In activated memory T cells (CD45RO CD4 lymphocyte) or CD4 Th1 or Th2 cells, resting CD4 cells (CTs=40), the expression of CG104210-01 is strongly down regulated suggesting a role for this putative protein in differentiation or activation of naive T cells. Therefore, expression of this gene may be used to distinguish this sample from other samples used in this panel. In addition, Therefore modulation of the expression and/or activity of this putative protein encoded by this gene might be beneficial for the control of autoimmune diseases and T cell mediated diseases such as arthritis, psoriasis, IBD and asthma.

Furthermore, low expression of this gene is also seen in small airway epithelium, and PMA/ionomycin treated LAK cells. In addition, moderate expression of this gene is also seen in kidney and thymus. Therefore, therapeutic modulation of this gene product may be useful in the treatment of autoimmune and inflammatory diseases involving these cell and tissue types such as asthma, COPD, arthritis, psoriasis, IBD, lupus, viral and bacterial infection.

general oncology screening panel_v_{—}2.4 Summary: Ag4270 Highest expression of the CG104210-01 gene is detected in metastatic melanoma (CT=33). Significant expression of this gene is also seen in melanoma and a lung cancer (OD06850-03C) samples. Interestingly, expression of this gene in lung cancer is higher as compared to the adjacent control sample. Therefore, expression of this gene may be used as diagnostic marker for detection of melanoma and lung cancer. Furthermore, therapeutic modulation of this gene may be useful in the treatment of melanoma and lung cancer.

O. CG104251-01: Type III Membrane Protein

Expression of gene CG104251-01 was assessed using the primer-probe set Ag4280, described in Table OA. Results of the RTQ-PCR runs are shown in Tables OB, OC and OD.

TABLE OA

Probe Name Ag4280

| | | | | |
|---|---|---|---|---|
| | | | Start | SEQ ID |
| Primers | Sequences | Length | Position | No |

| | | | | |
|---|---|---|---|---|
| Forward | 5′-cctttatgcaaccaacatgg-3′ | 20 | 7 | 192 |
| Probe | TET-5′-ccatgtcctgttcttagtgcttgaatgtcc-3′TAMRA | 30 | 37 | 193 |
| Reverse | 5′-ggcttcttcagcttcaggtt-3′ | 20 | 68 | 194 |

TABLE OB
CNS_neurodegeneration_v1.0

| | Rel. Exp. (%) Ag4280, Run | | Rel. Exp. (%) Ag4280, Run |
|---|---|---|---|
| Tissue Name | 224075293 | Tissue Name | 224075293 |
| AD 1 Hippo | 11.3 | Control (Path) 3 | 3.5 |
| | | Temporal Ctx | |
| AD 2 Hippo | 18.6 | Control (Path) 4 | 15.3 |
| | | Temporal Ctx | |
| AD 3 Hippo | 7.9 | AD 1 Occipital Ctx | 6.0 |
| AD 4 Hippo | 6.9 | AD 2 Occipital Ctx | 0.0 |
| | | (Missing) | |
| AD 5 hippo | 54.3 | AD 3 Occipital Ctx | 5.3 |
| AD 6 Hippo | 100.0 | AD 4 Occipital Ctx | 5.9 |
| Control 2 Hippo | 14.8 | AD 5 Occipital Ctx | 7.4 |
| Control 4 Hippo | 13.6 | AD 6 Occipital Ctx | 33.9 |
| Control (Path) 3 Hippo | 9.9 | Control 1 Occipital | 2.6 |
| | | Ctx | |
| AD 1 Temporal Ctx | 10.8 | Control 2 Occipital | 30.6 |
| | | Ctx | |
| AD 2 Temporal Ctx | 7.1 | Control 3 Occipital | 10.4 |
| | | Ctx | |
| AD 3 Temporal Ctx | 5.5 | Control 4 Occipital | 6.5 |
| | | Ctx | |
| AD 4 Temporal Ctx | 7.4 | Control (Path) 1 | 51.4 |
| | | Occipital Ctx | |
| AD 5 Inf Temporal Ctx | 47.0 | Control (Path) 2 | 13.8 |
| | | Occipital Ctx | |
| AD 5 SupTemporal Ctx | 37.1 | Control (Path) 3 | 5.2 |
| | | Occipital Ctx | |
| AD 6 Inf Temporal Ctx | 42.0 | Control (Path) 4 | 12.7 |
| | | Occipital Ctx | |
| AD 6 Sup Temporal Ctx | 50.0 | Control 1 Parietal Ctx | 7.2 |
| Control 1 Temporal Ctx | 4.4 | Control 2 Parietal Ctx | 29.1 |
| Control 2 Temporal Ctx | 27.0 | Control 3 Parietal Ctx | 23.3 |
| Control 3 Temporal Ctx | 8.8 | Control (Path) 1 | 33.7 |
| | | Parietal Ctx | |
| Control 4 Temporal Ctx | 2.9 | Control (Path) 2 | 6.6 |
| | | Parietal Ctx | |
| Control (Path) 1 | 44.4 | Control (Path) 3 | 14.2 |
| Temporal Ctx | | Parietal Ctx | |
| Control (Path) 2 | 26.1 | Control (Path) 4 | 16.5 |
| Temporal Ctx | | Parietal Ctx | |

TABLE OC
General_screening_panel_v1.4

| | Rel. Exp. (%) Ag4280, | | Rel. Exp. (%) Ag4280, |
|---|---|---|---|
| Tissue Name | Run 222183179 | Tissue Name | Run 222183179 |
| Adipose | 3.0 | Renal ca. TK-10 | 60.7 |
| Melanoma* | 43.5 | Bladder | 23.5 |
| Hs688(A).T | | | |
| Melanoma* | 45.1 | Gastric ca. (liver met.) | 56.6 |
| Hs688(B).T | | NCI-N87 | |
| Melanoma* M14 | 23.0 | Gastric ca. KATO III | 52.9 |
| Melanoma* LOXIMVI | 20.0 | Colon ca. SW-948 | 19.3 |
| Melanoma* SK-MEL-5 | 41.5 | Colon ca. SW480 | 48.3 |
| Squamous cell | 21.0 | Colon ca.* (SW480 met) | 34.4 |
| carcinoma SCC-4 | | SW620 | |
| Testis Pool | 6.1 | Colon ca. HT29 | 41.2 |
| Prostate ca.* (bone met) | 46.0 | Colon ca. HCT-116 | 100.0 |
| PC-3 | | | |
| Prostate Pool | 5.2 | Colon ca. CaCo-2 | 35.4 |
| Placenta | 7.5 | Colon cancer tissue | 30.4 |
| Uterus Pool | 1.3 | Colon ca. SW1116 | 13.4 |
| Ovarian ca. OVCAR-3 | 88.3 | Colon ca. Colo-205 | 15.8 |
| Ovarian ca. SK-OV-3 | 52.1 | Colon ca. SW-48 | 11.1 |
| Ovarian ca. OVCAR-4 | 17.7 | Colon Pool | 4.3 |
| Ovarian ca. OVCAR-5 | 80.1 | Small Intestine Pool | 2.6 |
| Ovarian ca. IGROV-1 | 51.8 | Stomach Pool | 8.7 |
| Ovarian ca. OVCAR-8 | 29.3 | Bone Marrow Pool | 2.0 |
| Ovary | 8.4 | Fetal Heart | 6.4 |
| Breast ca. MCF-7 | 31.6 | Heart Pool | 2.1 |
| Breast ca. MDA-MB- | 38.4 | Lymph Node Pool | 7.7 |
| 231 | | | |
| Breast ca. BT 549 | 19.8 | Fetal Skeletal Muscle | 3.5 |
| Breast ca. T47D | 79.6 | Skeletal Muscle Pool | 3.0 |
| Breast ca. MDA-N | 23.3 | Spleen Pool | 5.4 |
| Breast Pool | 8.6 | Thymus Pool | 11.5 |
| Trachea | 13.3 | CNS cancer (glio/astro) | 55.5 |
| | | U87-MG | |
| Lung | 3.8 | CNS cancer (glio/astro) U- | 50.3 |
| | | 118-MG | |
| Fetal Lung | 11.5 | CNS cancer (neuro; met) | 80.7 |
| | | SK-N-AS | |
| Lung ca. NCI-N417 | 13.1 | CNS cancer (astro) SF-539 | 37.1 |
| Lung ca. LX-1 | 32.1 | CNS cancer (astro) SNB- | 61.1 |
| | | 75 | |
| Lung ca. NCI-H146 | 9.9 | CNS cancer (glio) SNB-19 | 38.7 |
| Lung ca. SHP-77 | 23.5 | CNS cancer (glio) SF-295 | 47.0 |
| Lung ca. A549 | 40.1 | Brain (Amygdala) Pool | 2.3 |
| Lung ca. NCI-H526 | 8.7 | Brain (cerebellum) | 2.8 |
| Lung ca. NCI-H23 | 46.0 | Brain (fetal) | 3.1 |
| Lung ca. NCI-H460 | 23.2 | Brain (Hippocampus) Pool | 1.8 |
| Lung ca. HOP-62 | 27.7 | Cerebral Cortex Pool | 1.5 |
| Lung ca. NCI-H522 | 18.6 | Brain (Substantia nigra) | 1.9 |
| | | Pool | |
| Liver | 1.6 | Brain (Thalamus) Pool | 3.4 |
| Fetal Liver | 17.0 | Brain (whole) | 2.6 |
| Liver ca. HepG2 | 51.8 | Spinal Cord Pool | 2.8 |
| Kidney Pool | 8.8 | Adrenal Gland | 6.1 |
| Fetal Kidney | 13.2 | Pituitary gland Pool | 5.2 |
| Renal ca. 786-0 | 70.2 | Salivary Gland | 4.4 |
| Renal ca. A498 | 21.6 | Thyroid (female) | 7.5 |
| Renal ca. ACHN | 15.6 | Pancreatic ca. CAPAN2 | 52.1 |
| Renal ca. UO-31 | 27.4 | Pancreas Pool | 13.7 |

TABLE OD
Panel 4.1D

| | Rel. Exp. (%) Ag4280, | | Rel. Exp. (%) Ag4280, |
|---|---|---|---|
| Tissue Name | Run 176282949 | Tissue Name | Run 176282949 |
| Secondary Th1 act | 20.6 | HUVEC IL-1beta | 33.0 |
| Secondary Th2 act | 28.5 | HUVEC IFN gamma | 27.4 |
| Secondary Tr1 act | 23.7 | HUVEC TNF alpha + IFN | 18.2 |
| | | gamma | |
| Secondary Th1 rest | 2.8 | HUVEC TNF alpha + IL4 | 22.5 |
| Secondary Th2 rest | 6.8 | HUVEC IL-11 | 9.8 |
| Secondary Tr1 rest | 7.9 | Lung Microvascular EC none | 33.4 |
| Primary Th1 act | 10.3 | Lung Microvascular EC | 22.8 |
| | | TNF alpha + IL-1beta | |
| Primary Th2 act | 18.8 | Microvascular Dermal EC | 13.8 |
| | | none | |
| Primary Tr1 act | 15.9 | Microsvasular Dermal EC | 10.7 |
| | | TNF alpha + IL-1beta | |
| Primary Th1 rest | 3.4 | Bronchial epithelium | 17.0 |
| | | TNF alpha + IL1beta | |
| Primary Th2 rest | 3.9 | Small airway epithelium none | 5.8 |
| Primary Tr1 rest | 5.2 | Small airway epithelium | 8.4 |
| | | TNF alpha + IL-1beta | |
| CD45RA CD4 | 11.2 | Coronery artery SMC rest | 25.0 |
| lymphocyte act | | | |
| CD45RO CD4 | 16.7 | Coronery artery SMC | 25.9 |
| lymphocyte act | | TNF alpha + IL-1beta | |
| CD8 lymphocyte act | 14.6 | Astrocytes rest | 18.3 |
| Secondary CD8 | 11.1 | Astrocytes TNF alpha + IL- | 17.1 |
| lymphocyte rest | | 1beta | |
| Secondary CD8 | 5.5 | KU-812 (Basophil) rest | 19.9 |
| lymphocyte act | | | |
| CD4 lymphocyte none | 1.8 | KU-812 (Basophil) | 21.6 |
| | | PMA/ionomycin | |
| 2ry Th1/Th2/Tr1_anti- | 4.9 | CCD1106 (Keratinocytes) | 14.6 |
| CD95 CH11 | | none | |
| LAK cells rest | 10.2 | CCD1106 (Keratinocytes) | 15.2 |
| | | TNF alpha + IL-1beta | |
| LAK cells IL-2 | 6.7 | Liver cirrhosis | 1.1 |
| LAK cells IL-2 + IL-12 | 9.1 | NCI-H292 none | 11.8 |
| LAK cells IL-2 + IFN | 7.7 | NCI-H292 IL-4 | 15.3 |
| gamma | | | |
| LAK cells IL-2 + IL-18 | 8.3 | NCI-H292 IL-9 | 14.4 |
| LAK cells | 11.2 | NCI-H292 IL-13 | 14.8 |
| PMA/ionomycin | | | |
| NK Cells IL-2 rest | 9.4 | NCI-H292 IFN gamma | 11.7 |
| Two Way MLR 3 day | 8.6 | HPAEC none | 12.5 |
| Two Way MLR 5 day | 7.3 | HPAEC TNF alpha + IL-1beta | 42.9 |
| Two Way MLR 7 day | 8.0 | Lung fibroblast none | 10.7 |
| PBMC rest | 2.0 | Lung fibroblast TNF alpha + | 12.3 |
| | | IL-1beta | |
| PBMC PWM | 9.5 | Lung fibroblast IL-4 | 14.4 |
| PBMC PHA-L | 5.0 | Lung fibroblast IL-9 | 18.4 |
| Ramos (B cell) none | 21.6 | Lung fibroblast IL-13 | 15.4 |
| Ramos (B cell) ionomycin | 28.3 | Lung fibroblast IFN gamma | 26.2 |
| B lymphocytes PWM | 6.0 | Dermal fibroblast CCD1070 | 31.4 |
| | | rest | |
| B lymphocytes CD40L | 8.8 | Dermal fibroblast CCD1070 | 31.9 |
| and IL-4 | | TNF alpha | |
| EOL-1 dbcAMP | 10.6 | Dermal fibroblast CCD1070 | 26.2 |
| | | IL-1beta | |
| EOL-1 dbcAMP | 12.7 | Dermal fibroblast IFN gamma | 18.2 |
| PMA/ionomycin | | | |
| Dendritic cells none | 8.9 | Dermal fibroblast IL-4 | 20.7 |
| Dendritic cells LPS | 5.9 | Dermal fibroblast rest | 10.4 |
| Dendritic cells anti-CD40 | 7.6 | Neutrophils TNFa + LPS | 1.8 |
| Monocytes rest | 5.3 | Neutrophils rest | 2.6 |
| Monocytes LPS | 10.8 | Colon | 7.7 |
| Macrophages rest | 10.2 | Lung | 12.2 |
| Macrophages LPS | 2.7 | Thymus | 21.6 |
| HUVEC none | 16.0 | Kidney | 100.0 |
| HUVEC starved | 26.6 | | |

CNS_neurodegeneration_v1.0 Summary: Ag4280 Very low levels of expression of the CG104251-01 gene is seen in the brains of an independent group of individuals, with highest expression in hippocampus of an Alzeimer patient (CT=34.3). However, no differential expression of this gene was detected between Alzheimer's diseased postmortem brains and those of non-demented controls in this experiment. Please see Panel 1.4 for a discussion of the potential use of this gene in treatment of central nervous system disorders.

General_screening_panel_v1.4 Summary: Ag4280 Highest expression of the CG104251-01 gene is detected in a colon cancer HCT-116 cell line (CT=30). Significant expression of this gene is also seen in clusters of cancer cell lines derived from pancreatic, gastric, colon, renal, lung, breast, ovarian, prostate, squamous cell carcinoma, melanoma and brain cancers. Thus, expression of this gene may be useful as a marker to detect the presence of these cancers. Furthermore, therapeutic modulation of the expression or function of this gene product may be effective in the treatment of these cancers.

Among tissues with metabolic or endocrine function, this gene is expressed at low levels in pancreas, adrenal gland, thyroid, pituitary gland, fetal skeletal muscle, heart, fetal liver and the gastrointestinal tract. Therefore, therapeutic modulation of the activity of this gene may prove useful in the treatment of endocrine/metabolically related diseases, such as obesity and diabetes.

Interestingly, this gene is expressed at much higher levels in fetal (CT=32.7) when compared to adult liver (CT=36). This observation suggests that expression of this gene can be used to distinguish fetal from adult liver. In addition, the relative overexpression of this gene in fetal liver suggests that the protein product may enhance liver growth or development in the fetus and thus may also act in a regenerative capacity in the adult. Therefore, therapeutic modulation of the protein encoded by this gene could be useful in treatment of liver related diseases.

In addition, low expression of this gene is also seen in brain (thalamus). Therefore, therapeutic modulation of this gene product may be beneficial in the treatment of neurological disorders.

Panel 4.1D Summary: Ag4280 Highest expression of the CG104251-01 gene is detected in kidney (CT=31.8). This gene is expressed at low levels in a wide range of cell types of significance in the immune response in health and disease. These cells include members of the T-cell, B-cell, and endothelial cell family, as well as epithelial and fibroblast cell types from lung and skin, and normal tissues represented by lung, thymus and kidney. This pattern of expression suggests that this gene product may be involved in homeostatic processes for these and other cell types and tissues. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

P. CG104934-01: Potential Phospholipid-Transporting ATPase IH

Expression of gene CG104934-01 was assessed using the primer-probe set Ag4274, described in Table PA. Results of the RTQ-PCR runs are shown in Tables PB, PC and PD.

TABLE PA

Probe Name Ag4274

| | | | Start | SEQ ID |
|---|---|---|---|---|
| Primers | Sequences | Length | Position | No |
| Forward | 5′-tgcttcatcttccctcagttt-3′ | 21 | 2734 | 195 |
| Probe | TET-5′-acaacagactttgtacgacaccgcgt-3′-TAMRA | 26 | 2781 | 196 |
| Reverse | 5′-gctgatgttgtagagggtcaga-3′ | 22 | 2808 | 197 |

TABLE PB
CNS_neurodegeneration_v1.0

| | Rel. Exp. (%) Ag4274, Run | | Rel. Exp. (%) Ag4274, Run |
|---|---|---|---|
| Tissue Name | 224075762 | Tissue Name | 224075762 |
| AD 1 Hippo | 24.5 | Control (Path) 3 | 6.9 |
| | | Temporal Ctx | |
| AD 2 Hippo | 39.5 | Control (Path) 4 | 27.5 |
| | | Temporal Ctx | |
| AD 3 Hippo | 11.1 | AD 1 Occipital Ctx | 27.5 |
| AD 4 Hippo | 9.3 | AD 2 Occipital Ctx | 0.0 |
| | | (Missing) | |
| AD 5 Hippo | 86.5 | AD 3 Occipital Ctx | 13.2 |
| AD 6 Hippo | 71.2 | AD 4 Occipital Ctx | 16.0 |
| Control 2 Hippo | 27.9 | AD 5 Occipital Ctx | 31.4 |
| Control 4 Hippo | 17.4 | AD 6 Occipital Ctx | 35.1 |
| Control (Path) 3 Hippo | 13.6 | Control 1 Occipital Ctx | 0.0 |
| AD 1 Temporal Ctx | 42.6 | Control 2 Occipital Ctx | 50.3 |
| AD 2 Temporal Ctx | 34.6 | Control 3 Occipital Ctx | 39.2 |
| AD 3 Temporal Ctx | 12.5 | Control 4 Occipital Ctx | 13.2 |
| AD 4 Temporal Ctx | 36.1 | Control (Path) 1 | 100.0 |
| | | Occipital Ctx | |
| AD 5 Inf Temporal Ctx | 77.9 | Control (Path) 2 | 21.6 |
| | | Occipital Ctx | |
| AD 5 Sup Temporal | 48.0 | Control (Path) 3 | 12.3 |
| Ctx | | Occipital Ctx | |
| AD 6 Inf Temporal Ctx | 97.9 | Control (Path) 4 | 22.2 |
| | | Occipital Ctx | |
| AD 6 Sup Temporal | 66.0 | Control 1 Parietal Ctx | 10.5 |
| Ctx | | | |
| Control 1 Temporal | 7.7 | Control 2 Parietal Ctx | 47.0 |
| Ctx | | | |
| Control 2 Temporal | 25.7 | Control 3 Parietal Ctx | 24.7 |
| Ctx | | | |
| Control 3 Temporal | 21.6 | Control (Path) 1 | 53.2 |
| Ctx | | Parietal Ctx | |
| Control 3 Temporal | 10.6 | Control (Path) 2 | 25.0 |
| Ctx | | Parietal Ctx | |
| Control (Path) 1 | 42.0 | Control (Path) 3 | 12.3 |
| Temporal Ctx | | Parietal Ctx | |
| Control (Path) 2 | 29.3 | Control (Path) 4 | 40.6 |
| Temporal Ctx | | Parietal Ctx | |

TABLE PC
General_screening_panel_v1.4

| | Rel. Exp. (%) Ag4274, | | Rel. Exp. (%) Ag4274, |
|---|---|---|---|
| Tissue Name | Run 222182089 | Tissue Name | Run 222182089 |
| Adipose | 4.8 | Renal ca. TK-10 | 44.8 |
| Melanoma* | 4.1 | Bladder | 14.5 |
| Hs688(A).T | | | |
| Melanoma* | 6.0 | Gastric ca. (liver met.) | 18.9 |
| Hs688(B).T | | NCI-N87 | |
| Melanoma* M14 | 27.5 | Gastric ca. KATO III | 64.6 |
| Melanoma* LOXIMVI | 0.0 | Colon ca. SW-948 | 6.4 |
| Melanoma* SK-MEL-5 | 33.2 | Colon ca. SW480 | 25.3 |
| Squamous cell | 9.2 | Colon ca.* (SW480 met) | 26.6 |
| carcinoma SCC-4 | | SW620 | |
| Testis Pool | 7.4 | Colon ca. HT29 | 10.1 |
| Prostate ca.* (bone met) | 20.2 | Colon ca. HCT-116 | 26.2 |
| PC-3 | | | |
| Prostate Pool | 2.1 | Colon ca. CaCo-2 | 44.1 |
| Placenta | 7.9 | Colon cancer tissue | 20.9 |
| Uterus Pool | 3.6 | Colon ca. SW1116 | 4.0 |
| Ovarian ca. OVCAR-3 | 15.8 | Colon ca. Colo-205 | 20.9 |
| Ovarian ca. SK-OV-3 | 38.7 | Colon ca. SW-48 | 8.1 |
| Ovarian ca. OVCAR-4 | 14.0 | Colon Pool | 14.3 |
| Ovarian ca. OVCAR-5 | 19.3 | Small Intestine Pool | 8.8 |
| Ovarian ca. IGROV-1 | 9.3 | Stomach Pool | 5.9 |
| Ovarian ca. OVCAR-8 | 4.1 | Bone Marrow Pool | 3.5 |
| Ovary | 5.4 | Fetal Heart | 15.8 |
| Breast ca. MCF-7 | 8.0 | Heart Pool | 8.4 |
| Breast ca. MDA-MB- | 17.8 | Lymph Node Pool | 14.0 |
| 231 | | | |
| Breast ca. BT 549 | 20.7 | Fetal Skeletal Muscle | 3.1 |
| Breast ca. T47D | 28.5 | Skeletal Muscle Pool | 6.1 |
| Breast ca. MDA-N | 0.0 | Spleen Pool | 7.0 |
| Breast Pool | 15.2 | Thymus pool | 8.8 |
| Trachea | 6.5 | CNS cancer (glio/astro) | 1.0 |
| | | U87-MG | |
| Lung | 1.5 | CNS cancer (glio/astro) U- | 0.0 |
| | | 118-MG | |
| Fetal Lung | 100.0 | CNS cancer (neuro; met) | 4.6 |
| | | SK-N-AS | |
| Lung ca. NCI-N417 | 2.2 | CNS cancer (astro) SF-539 | 3.8 |
| Lung ca. LX-1 | 48.6 | CNS cancer (astro) SNB- | 15.1 |
| | | 75 | |
| Lung ca. NCI-H146 | 7.7 | CNS cancer (glio) SNB-19 | 11.1 |
| Lung ca. SHP-77 | 17.4 | CNS cancer (glio) SF-295 | 10.4 |
| Lung ca. A549 | 10.8 | Brain (Amygdala) Pool | 5.6 |
| Lung ca. NCI-H526 | 3.7 | Brain (cerebellum) | 10.4 |
| Lung ca. NCI-H23 | 12.8 | Brain (fetal) | 6.3 |
| Lung ca. NCI-H460 | 12.5 | Brain (Hippocampus) Pool | 5.6 |
| Lung ca. HOP-62 | 4.0 | Cerebral Cortex Pool | 7.1 |
| Lung ca. NCI-H522 | 5.8 | Brain (Substantia nigra) | 6.2 |
| | | Pool | |
| Liver | 2.0 | Brain (Thalamus) Pool | 6.9 |
| Fetal Liver | 10.7 | Brain (whole) | 7.5 |
| Liver ca. HepG2 | 18.6 | Spinal Cord Pool | 10.2 |
| Kidney Pool | 12.8 | Adrenal Gland | 13.2 |
| Fetal Kidney | 11.1 | Pituitary gland Pool | 6.1 |
| Renal ca. 786-0 | 17.0 | Salivary Gland | 2.7 |
| Renal ca. A498 | 11.6 | Thyroid (female) | 3.2 |
| Renal ca. ACHN | 12.2 | Pancreatic ca. CAPAN2 | 26.1 |
| Renal ca. UO-31 | 10.2 | Pancreas Pool | 22.5 |

TABLE PD
Panel 4.1D

| | Rel. Exp. (%) Ag4274, | | Rel. Exp. (%) Ag4274, |
|---|---|---|---|
| Tissue Name | Run 176243763 | Tissue Name | Run 176243763 |
| Secondary Th1 act | 58.2 | HUVEC IL-1beta | 34.9 |
| Secondary Th2 act | 55.9 | HUVEC IFN gamma | 54.3 |
| Secondary Tr1 act | 44.1 | HUVEC TNF alpha + IFN | 15.8 |
| | | gamma | |
| Secondary Th1 rest | 16.3 | HUVEC TNF alpha + IL4 | 31.4 |
| Secondary Th2 rest | 14.2 | HUVEC IL-11 | 30.4 |
| Secondary Tr1 rest | 24.1 | Lung Microvascular EC none | 44.4 |
| Primary Th1 act | 26.4 | Lung Microvascular EC | 23.8 |
| | | TNF alpha + IL-1beta | |
| Primary Th2 act | 47.6 | Microvascular Dermal EC | 37.1 |
| | | none | |
| Primary Tr1 act | 36.3 | Microsvasular Dermal EC | 33.9 |
| | | TNF alpha + IL-1beta | |
| Primary Th1 rest | 15.3 | Bronchial epithelium | 17.0 |
| | | TNF alpha + IL1beta | |
| Primary Th2 rest | 11.3 | Small airway epithelium none | 14.3 |
| Primary Tr1 rest | 25.5 | Small airway epithelium | 27.4 |
| | | TNF alpha + IL-1beta | |
| CD45RA CD4 | 24.5 | Coronery artery SMC rest | 23.3 |
| lymphocyte act | | | |
| CD45RO CD4 | 42.0 | Coronery artery SMC | 20.3 |
| lymphocyte act | | TNF alpha + IL-1beta | |
| CD8 lymphocyte act | 28.5 | Astrocytes rest | 15.3 |
| Secondary CD8 | 36.1 | Astrocytes TNF alpha + IL- | 10.0 |
| lymphocyte rest | | 1beta | |
| Secondary CD8 | 12.7 | KU-812 (Basophil) rest | 50.7 |
| lymphocyte act | | | |
| CD4 lymphocyte none | 14.9 | KU-812 (Basophil) | 56.6 |
| | | PMA/ionomycin | |
| 2ry Th1/Th2/Tr1_anti- | 31.9 | CCD1106 (Keratinocytes) | 36.1 |
| CD95 CH11 | | none | |
| LAK cells rest | 55.1 | CCD1106 (Keratinocytes) | 27.7 |
| | | TNF alpha + IL-1beta | |
| LAK cells IL-2 | 26.2 | Liver cirrhosis | 15.7 |
| LAK cells IL-2 + IL-12 | 22.4 | NCI-H292 none | 25.7 |
| LAK cells IL-2 + IFN | 13.3 | NCI-H292 IL-4 | 58.2 |
| gamma | | | |
| LAK cells IL-2 + IL-18 | 19.2 | NCI-H292 IL-9 | 48.0 |
| LAK cells | 26.4 | NCI-H292 IL-13 | 63.3 |
| PMA/ionomycin | | | |
| NK Cells IL-2 rest | 26.1 | NCI-H292 IFN gamma | 29.7 |
| Two Way MLR 3 day | 33.4 | HPAEC none | 29.3 |
| Two Way MLR 5 day | 39.8 | HPAEC TNF alpha + IL-1beta | 51.8 |
| Two Way MLR 7 day | 21.8 | Lung fibroblast none | 25.7 |
| PBMC rest | 23.2 | Lung fibroblast TNF alpha + | 20.0 |
| | | IL-1beta | |
| PBMC PWM | 25.9 | Lung fibroblast IL-4 | 42.9 |
| PBMC PHA-L | 30.8 | Lung fibroblast IL-9 | 36.3 |
| Ramos (B cell) none | 0.0 | Lung fibroblast IL-13 | 48.0 |
| Ramos (B cell) ionomycin | 0.0 | Lung fibroblast IFN gamma | 25.5 |
| B lymphocytes PWM | 16.5 | Dermal fibroblast CCD1070 | 17.7 |
| | | rest | |
| B lymphocytes CD40L | 17.4 | Dermal fibroblast CCD1070 | 30.1 |
| and IL-4 | | TNF alpha | |
| EOL-1 dbcAMP | 65.5 | Dermal fibroblast CCD1070 | 10.7 |
| | | IL-1beta | |
| EOL-1 dbcAMP | 66.4 | Dermal fibroblast IFN gamma | 20.7 |
| PMA/ionomycin | | | |
| Dendritic cells none | 58.6 | Dermal fibroblast IL-4 | 32.1 |
| Dendritic cells LPS | 46.0 | Dermal fibroblasts rest | 16.2 |
| Dendritic cells anti-CD40 | 65.1 | Neutrophils TNFa + LPS | 36.1 |
| Monocytes rest | 95.9 | Neutrophils rest | 73.2 |
| Monocytes LPS | 100.0 | Colon | 9.2 |
| Macrophages rest | 65.5 | Lung | 62.0 |
| Macrophages LPS | 45.7 | Thymus | 25.9 |
| HUVEC none | 28.1 | Kidney | 51.1 |
| HUVEC starved | 45.7 | | |

CNS_neurodegeneration_v1.0 Summary: Ag4274 This panel confirms the expression of the CG104934-01 gene at low levels in the brain in an independent group of individuals. This gene is found to be upregulated in the temporal cortex of Alzheimer's disease patients. Blockade of this receptor may be of use in the treatment of this disease and decrease neuronal death.

General_screening_panel_v1.4 Summary: Ag4274 Highest expression of the CG104934-01 gene is detected in fetal lung (CT=23.5). Interestingly, this gene is expressed at much higher levels in fetal (CT=23.5) when compared to adult lung (CT=29.5). This observation suggests that expression of this gene can be used to distinguish fetal from adult lung. In addition, the relative overexpression of this gene in fetal lung suggests that the protein product may enhance lung growth or development in the fetus and thus may also act in a regenerative capacity in the adult. Therefore, therapeutic modulation of the protein encoded by this gene could be useful in treatment of lung related diseases.

Significant expression of this gene is also seen in clusters of cancer cell lines derived from pancreatic, gastric, colon, renal, lung, breast, ovarian, prostate, squamous cell carcinoma, melanoma and brain cancers. Thus, expression of this gene may be used as a diagnostic marker for detection of these cancers. Furthermore, therapeutic modulation of the expression or function of this gene may be effective in the treatment of pancreatic, gastric, colon, renal, lung, breast, ovarian, prostate, squamous cell carcinoma, melanoma and brain cancers.

Among tissues with metabolic or endocrine function, this gene is expressed at high levels in pancreas, adipose, adrenal gland, thyroid, pituitary gland, skeletal muscle, heart, liver and the gastrointestinal tract. Therefore, therapeutic modulation of the activity of this gene may prove useful in the treatment of endocrine/metabolically related diseases, such as obesity and diabetes.

The CG104934-01 gene codes for a variant of potential phospholipid-transporting ATPase, a P-type ATPase with phospholipid transporting activity. In mice, a P-type ATPase (p-locus fat-associated ATPase) was mapped to locus that deletion of which results in increase in the body fat of the mice (Dhar et al, 2000, Physiol Genomics 4(1):93-100, PMID: 11074018). Therefore, based on functional homology, the CG104934-01 gene may also play a role in modulation of the body fat in human and therapeutic modulation of this protein may be useful in the treatment of obesity and diabetes.

Mutations in the FIC1 gene, a member of phospholipid-transporting ATPase, is shown to constitute the molecular defect in familial intrahepatic cholestasis I (Byler's disease) and benign recurrent intrahepatic cholestasis (Ujhazy et al., 2001, Hepatology 34:768-75, PMID: 11584374). Thus, based on homology, potential phospholipid-transporting ATPase encoded by this gene may also play a role in pathology of Byler's disease and intrahepatic cholestasis and therapeutic modulation of this protein may be useful in the treatment of these diseases.

In addition, this gene is expressed at high levels in all regions of the central nervous system examined, including amygdala, hippocampus, substantia nigra, thalamus, cerebellum, cerebral cortex, and spinal cord. Therefore, this gene may play a role in central nervous system disorders such as Alzheimer's disease, Parkinson's disease, epilepsy, multiple sclerosis, schizophrenia and depression.

Panel 4.1D Summary: Ag4274 Highest expression of the CG104934-01 gene is detected in monocytes (CTs=28.4). This gene is expressed at high to moderate levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. This pattern is in agreement with the expression profile in General_screening_panel_v1.4 and also suggests a role for the gene product in cell survival and proliferation. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

Q. CG105463-01 and CG105463-02: Meningioma-Expressed Antigen 6/11 (MEA6) (MEA11)

Expression of gene CG105463-01 and CG105463-02 was assessed using the primer-probe set Ag4288, described in Table QA. Results of the RTQ-PCR runs are shown in Tables QB, QC and QD. Please note that CG105463-02 represents a full-length physical clone of the CG105463-01 gene, validating the prediction of the gene sequence.

TABLE QA

Probe Name Ag4288

| | | | Start | SEQ ID |
|---|---|---|---|---|
| Primers | Sequences | Length | Position | No |
| Forward | 5′-agactccaaagtacacgcagaa-3′ | 22 | 834 | 198 |
| Probe | TET-5′-tcacatcgagactctgactgaacgct-3′- | 26 | 879 | 199 |
| Reverse | 5′-gcctgatctttgatctttagca-3′ | 22 | 905 | 200 |

TABLE QB
CNS_neurodegeneration_v1.0

| | Rel. Exp. | | Rel. Exp. |
|---|---|---|---|
| | (%) Ag4288, Run | | (%) Ag4288, Run |
| Tissue Name | 224064582 | Tissue Name | 224064582 |
| AD 1 Hippo | 9.7 | Control (Path) 3 | 5.4 |
| | | Temporal Ctx | |
| AD 2 Hippo | 15.1 | Control (Path) 4 | 65.5 |
| | | Temporal Ctx | |
| AD 3 Hippo | 15.2 | AD 1 Occipital Ctx | 28.3 |
| AD 4 Hippo | 13.9 | AD 2 Occipital Ctx | 0.0 |
| | | (Missing) | |
| AD 5 Hippo | 63.3 | AD 3 Occipital Ctx | 2.9 |
| AD 6 Hippo | 68.3 | AD 4 Occipital Ctx | 21.8 |
| Control 2 Hippo | 13.8 | AD 5 Occipital Ctx | 19.3 |
| Control 4 Hippo | 7.7 | AD 6 Occipital Ctx | 16.5 |
| Control (Path) 3 Hippo | 7.0 | Control 1 Occipital Ctx | 2.7 |
| AD 1 Temporal Ctx | 18.3 | Control 2 Occipital Ctx | 20.3 |
| AD 2 Temporal Ctx | 15.1 | Control 3 Occipital Ctx | 30.6 |
| AD 3 Temporal Ctx | 7.9 | Control 4 Occipital Ctx | 9.5 |
| AD 4 Temporal Ctx | 24.0 | Control (Path) 1 | 41.5 |
| | | Occipital Ctx | |
| AD 5 Inf Temporal Ctx | 62.4 | Control (Path) 2 | 23.5 |
| | | Occipital Ctx | |
| AD 5 Sup Temporal | 44.1 | Control (Path) 3 | 4.6 |
| Ctx | | Occipital Ctx | |
| AD 6 Inf Temporal Ctx | 77.4 | Control (Path) 4 | 47.6 |
| | | Occipital Ctx | |
| AD 6 Sup Temporal | 100.0 | Control 1 Parietal Ctx | 7.6 |
| Ctx | | | |
| Control 1 Temporal | 13.7 | Control 2 Parietal Ctx | 46.0 |
| Ctx | | | |
| Control 2 Temporal | 5.9 | Control 3 Parietal Ctx | 7.6 |
| Ctx | | | |
| Control 3 Temporal | 11.7 | Control (Path) 1 | 33.0 |
| Ctx | | Parietal Ctx | |
| Control 3 Temporal | 20.3 | Control (Path) 2 | 41.5 |
| Ctx | | Parietal Ctx | |
| Control (Path) 1 | 36.1 | Control (Path) 3 | 6.5 |
| Temporal Ctx | | Parietal Ctx | |
| Control (Path) 2 | 21.8 | Control (Path) 4 | 66.4 |
| Temporal Ctx | | Parietal Ctx | |

TABLE QC
General_screening_panel_v1.4

| | Rel. Exp. | | Rel. Exp. |
|---|---|---|---|
| | (%) Ag4288, Run | | (%) Ag4288, Run |
| Tissue Name | 222182748 | Tissue Name | 222182748 |
| Adipose | 11.0 | Renal ca. TK-10 | 40.1 |
| Melanoma* | 2.6 | Bladder | 6.7 |
| Hs688(A).T | | | |
| Melanoma* | 1.9 | Gastric ca. (liver met.) | 3.2 |
| Hs688(B).T | | NCI-N87 | |
| Melanoma* M14 | 1.5 | Gastric ca. KATO III | 1.6 |
| Melanoma* LOXIMVI | 0.0 | Colon ca. SW-948 | 5.8 |
| Melanoma* SK-MEL-5 | 4.8 | Colon ca. SW480 | 15.7 |
| Squamous cell | 1.4 | Colon ca.* (SW480 met) | 8.3 |
| carcinoma SCC-4 | | SW620 | |
| Testis Pool | 95.9 | Colon ca. HT29 | 4.1 |
| Prostate ca.* (bone met) | 3.5 | Colon ca. HCT-116 | 16.4 |
| PC-3 | | | |
| Prostate Pool | 4.5 | Colon ca. CaCo-2 | 100.0 |
| Placenta | 0.6 | Colon cancer tissue | 17.6 |
| Uterus Pool | 2.7 | Colon ca. SW1116 | 1.9 |
| Ovarian ca. OVCAR-3 | 2.1 | Colon ca. Colo-205 | 0.6 |
| Ovarian ca. SK-OV-3 | 18.7 | Colon ca. SW-48 | 5.5 |
| Ovarian ca. OVCAR-4 | 0.5 | Colon Pool | 5.4 |
| Ovarian ca. OVCAR-5 | 6.4 | Small Intestine Pool | 12.4 |
| Ovarian ca. IGROV-1 | 30.1 | Stomach Pool | 5.6 |
| Ovarian ca. OVCAR-8 | 0.0 | Bone Marrow Pool | 2.6 |
| Ovary | 2.8 | Fetal Heart | 18.4 |
| Breast ca. MCF-7 | 5.5 | Heart Pool | 0.8 |
| Breast ca. MDA-MB- | 1.4 | Lymph Node Pool | 12.8 |
| 231 | | | |
| Breast ca. BT 549 | 2.2 | Fetal Skeletal Muscle | 1.4 |
| Breast ca. T47D | 6.2 | Skeletal Muscle Pool | 9.9 |
| Breast ca. MDA-N | 1.1 | Spleen Pool | 4.2 |
| Breast Pool | 10.4 | Thymus Pool | 8.8 |
| Trachea | 7.3 | CNS cancer (glio/astro) | 0.6 |
| | | U87-MG | |
| Lung | 4.1 | CNS cancer (glio/astro) U- | 0.0 |
| | | 118-MG | |
| Fetal Lung | 10.1 | CNS cancer (neuro; met) | 12.0 |
| | | SK-N-AS | |
| Lung ca. NCI-N417 | 0.0 | CNS cancer (astro) SF-539 | 0.0 |
| Lung ca. LX-1 | 25.5 | CNS cancer (astro) SNB- | 6.1 |
| | | 75 | |
| Lung ca. NCI-H146 | 0.0 | CNS cancer (glio) SNB-19 | 34.6 |
| Lung ca. SHP-77 | 4.0 | CNS cancer (glio) SF-295 | 0.9 |
| Lung ca. A549 | 1.6 | Brain (Amygdala) Pool | 5.7 |
| Lung ca. NCI-H526 | 0.5 | Brain (cerebellum) | 5.8 |
| Lung ca. NCI-H23 | 5.4 | Brain (fetal) | 37.4 |
| Lung ca. NCI-H460 | 5.0 | Brain (Hippocampus) Pool | 12.2 |
| Lung ca. HOP-62 | 1.1 | Cerebral Cortex Pool | 9.4 |
| Lung ca. NCI-H522 | 1.6 | Brain (Substantia nigra) | 6.5 |
| | | Pool | |
| Liver | 0.0 | Brain (Thalamus) Pool | 12.5 |
| Fetal Liver | 41.5 | Brain (whole) | 4.0 |
| Liver ca. HepG2 | 62.0 | Spinal Cord Pool | 7.6 |
| Kidney Pool | 21.0 | Adrenal Gland | 0.0 |
| Fetal Kidney | 25.0 | Pituitary gland Pool | 0.6 |
| Renal ca. 786-0 | 0.5 | Salivary Gland | 0.6 |
| Renal ca. A498 | 5.0 | Thyroid (female) | 0.2 |
| Renal ca. ACHN | 0.5 | Pancreatic ca. CAPAN2 | 0.0 |
| Renal ca. UO-31 | 0.0 | Pancreas Pool | 12.1 |

TABLE QD
Panel 4.1D

| | Rel. Exp. (%) | | Rel. Exp. (%) |
|---|---|---|---|
| | Ag4288, Run | | Ag4288, Run |
| Tissue Name | 181981927 | Tissue Name | 181981927 |
| Secondary Th1 act | 0.4 | HUVEC IL-1beta | 0.5 |
| Secondary Th2 act | 0.6 | HUVEC IFN gamma | 0.0 |
| Secondary Tr1 act | 0.0 | HUVEC TNF alpha + IFN | 0.1 |
| | | gamma | |
| Secondary Th1 rest | 0.6 | HUVEC TNF alpha + IL4 | 0.0 |
| Secondary Th2 rest | 0.3 | HUVEC IL-11 | 0.0 |
| Secondary Tr1 rest | 0.0 | Lung Microvascular EC none | 1.9 |
| Primary Th1 act | 0.0 | Lung Microvascular EC | 0.5 |
| | | TNF alpha + IL-1beta | |
| Primary Th2 act | 0.1 | Microvascular Dermal EC | 0.0 |
| | | none | |
| Primary Tr1 act | 0.0 | Microsvasular Dermal EC | 0.0 |
| | | TNF alpha + IL-1beta | |
| Primary Th1 rest | 0.0 | Bronchial epithelium | 4.1 |
| | | TNF alpha + IL1beta | |
| Primary Th2 rest | 0.0 | Small airway epithelium none | 0.6 |
| Primary Tr1 rest | 0.0 | Small airway epithelium | 1.4 |
| | | TNF alpha + IL-1beta | |
| CD45RA CD4 | 0.0 | Coronery artery SMC rest | 0.0 |
| lymphocyte act | | | |
| CD45RO CD4 | 0.0 | Coronery artery SMC | 0.5 |
| lymphocyte act | | TNF alpha + IL-1beta | |
| CD8 lymphocyte act | 0.0 | Astrocytes rest | 2.3 |
| Secondary CD8 | 0.0 | Astrocytes TNF alpha + IL- | 0.8 |
| lymphocyte rest | | 1beta | |
| Secondary CD8 | 0.0 | KU-812 (Basophil) rest | 13.6 |
| lymphocyte act | | | |
| CD4 lymphocyte none | 0.3 | KU-812 (Basophil) | 13.5 |
| | | PMA/ionomycin | |
| 2ry Th1/Th2/Tr1_anti- | 0.0 | CCD1106 (Keratinocytes) | 0.5 |
| CD95 CH11 | | none | |
| LAK cells rest | 0.0 | CCD1106 (Keratinocytes) | 0.0 |
| | | TNF alpha + IL-1beta | |
| LAK cells IL-2 | 0.0 | Liver cirrhosis | 1.1 |
| LAK cells IL-2 + IL-12 | 0.0 | NCI-H292 none | 3.8 |
| LAK cells IL-2 + IFN | 0.0 | NCI-H292 IL-4 | 0.9 |
| gamma | | | |
| LAK cells IL-2 + IL-18 | 0.6 | NCI-H292 IL-9 | 1.0 |
| LAK cells | 0.0 | NCI-H292 IL-13 | 1.2 |
| PMA/ionomycin | | | |
| NK Cells IL-2 rest | 0.0 | NCI-H292 IFN gamma | 0.0 |
| Two Way MLR 3 day | 0.0 | HPAEC none | 1.9 |
| Two Way MLR 5 day | 0.0 | HPAEC TNF alpha + IL-1beta | 0.0 |
| Two Way MLR 7 day | 0.0 | Lung fibroblast none | 0.4 |
| PBMC rest | 0.7 | Lung fibroblast TNF alpha + | 0.9 |
| | | IL-1beta | |
| PBMC PWM | 0.0 | Lung fibroblast IL-4 | 0.4 |
| PBMC PHA-L | 0.4 | Lung fibroblast IL-9 | 0.9 |
| Ramos (B cell) none | 0.0 | Lung fibroblast IL-13 | 0.5 |
| Ramos (B cell) ionomycin | 0.0 | Lung fibroblast IFN gamma | 0.2 |
| B lymphocytes PWM | 1.5 | Dermal fibroblast CCD1070 | 0.0 |
| | | rest | |
| B lymphocytes CD40L | 0.0 | Dermal fibroblast CCD1070 | 1.5 |
| and IL-4 | | TNF alpha | |
| EOL-1 dbcAMP | 0.0 | Dermal fibroblast CCD1070 | 0.0 |
| | | IL-1beta | |
| EOL-1 dbcAMP | 0.0 | Dermal fibroblast IFN gamma | 0.7 |
| PMA/ionomycin | | | |
| Dendritic cells none | 0.9 | Dermal fibroblast IL-4 | 0.0 |
| Dendritic cells LPS | 0.0 | Dermal Fibroblasts rest | 0.5 |
| Dendritic cells anti-CD40 | 0.0 | Neutrophils TNFa + LPS | 0.4 |
| Monocytes rest | 0.0 | Neutrophils rest | 2.6 |
| Monocytes LPS | 0.8 | Colon | 6.7 |
| Macrophages rest | 0.0 | Lung | 1.8 |
| Macrophages LPS | 0.0 | Thymus | 8.8 |
| HUVEC none | 0.0 | Kidney | 100.0 |
| HUVEC starved | 0.0 | | |

CNS_neurodegeneration_v1.0 Summary: Ag4288 This panel confirms the expression of the CG105463-01 gene at low levels in the brains of an independent group of individuals. However, no differential expression of this gene was detected between Alzheimer's diseased postmortem brains and those of non-demented controls in this experiment. Please see Panel 1.4 for a discussion of the potential use of this gene in treatment of central nervous system disorders.

General_screening_panel_v1.4 Summary: Ag4288 Highest expression of the CG105463-01 gene is detected in colon cancer CaCo-2 cell line (CT=30). Significant expression is also seen in number of cancer cell lines derived from colon, renal, lung, liver, breast, ovarian, and brain cancers. Thus, expression of this gene as a marker to detect the presence of these cancers. Furthermore, therapeutic modulation of the expression or function of this gene may be effective in the treatment of colon, renal, lung, liver, breast, ovarian, and brain cancers.

The CG105463-01 gene codes for a homolog of meningioma-expressed antigen 6/11 (MEA6). MGEA6 is overexpressed in meningioma and glioma tumor cells. Furthermore, the immune response to MGEA6/11 is frequent in both meningioma and glioma patients (Comtesse et al., 2002, Oncogene 21(2):239-47, PMID: 11803467). Thus, based on the homology, MEA6 like protein encoded by the CG105463-01 gene may play a role in pathology of meningioma and glioma and therapeutic modulation of this gene may be beneficial in the treatment of these tumors.

Among tissues with metabolic or endocrine function, this gene is expressed at moderate to low levels in pancreas, adipose, skeletal muscle, fetal heart, fetal liver and the gastrointestinal tract. Therefore, therapeutic modulation of the activity of this gene may prove useful in the treatment of endocrine/metabolically related diseases, such as obesity and diabetes.

Interestingly, this gene is expressed at much higher levels in fetal (CTs=31-32) when compared to adult liver and heart(CTs>37). This observation suggests that expression of this gene can be used to distinguish fetal heart and liver from corresponding adult tissues. In addition, the relative overexpression of this gene in fetal tissue suggests that the protein product may enhance growth or development of heart and liver in the fetus and thus may also act in a regenerative capacity in the adult. Therefore, therapeutic modulation of MEA6 like protein encoded by this gene could be useful in treatment of heart and liver related diseases.

In addition, this gene is expressed at moderate levels in all regions of the central nervous system examined, including amygdala, hippocampus, substantia nigra, thalamus, cerebellum, cerebral cortex, and spinal cord. Therefore, this gene may play a role in central nervous system disorders such as Alzheimer's disease, Parkinson's disease, epilepsy, multiple sclerosis, schizophrenia and depression.

Panel 4.1D Summary: Ag4288 Highest expression of the CG105463-01 gene is detected in kidney (CT=29.3). Therefore, expression of this gene may be used to distinguish kidney from other samples used in this panel. Furthermore, therapeutic modulation of this gene product may be useful in the treatment of autoimmune and inflammatory disease that affect kidney, including lupus and glomerulonephritis.

In addition, moderate to low expression of this gene is also seen in TNFalpha+IL1beta treated bronchial epithelium, basophils, NCI-H292, resting neutrophils and normal tissues represented by colon and thymus. Therefore, therapeutic modulation of this gene product may be beneficial in the treatment of Crohn's disease, ulcerative colitis, multiple sclerosis, chronic obstructive pulmonary disease, asthma, emphysema, rheumatoid arthritis, lupus erythematosus, or psoriasis.

R. CG105491-01: Serine Protease

Expression of gene CG105491-01 was assessed using the primer-probe sets Ag4348, Ag4302 and Ag6953, described in Tables RA, RB and RC. Results of the RTQ-PCR runs are shown in Tables RD, RE and RF.

TABLE RA

Probe Name Ag4348

| | | | Start | SEQ ID |
|---|---|---|---|---|
| Primers | Sequences | Length | Position | No |
| Forward | 5′-acctgctctacggacacatgt-3′ | 21 | 669 | 201 |
| Probe | TET-5′-ctacatcatgcccgacatgctgtgt-3′-TAMRA | 25 | 691 | 202 |
| Reverse | 5′-ctcacacacggtcttagcattc-3′ | 22 | 730 | 203 |

TABLE RB

Probe Name Ag4302

| | | | Start | SEQ ID |
|---|---|---|---|---|
| Primers | Sequences | Length | Position | No |
| Forward | 5′-cctctgtaccctggagtgtatg-3′ | 22 | 839 | 204 |
| Probe | TET-5′-ccagtgtttcctatttctcaaaatgga-3′-TAMRA | 27 | 861 | 205 |
| Reverse | 5′-tgggcgtgatttctatgttatc-3′ | 22 | 893 | 206 |

TABLE RC

Probe Name Ag6953

| | | | Start | SEQ ID |
|---|---|---|---|---|
| Primers | Sequences | Length | Position | No |
| Forward | 5′-gaaataggaaacactggcataca-3′ | 23 | 855 | 207 |
| Probe | TET-5′-ctgctccaaccctctgtaccctggag-3′-TAMRA | 26 | 829 | 208 |
| Reverse | 5′-ggttgcagattggaattgtg-3′ | 20 | 795 | 209 |

TABLE RD
CNS_neurodegeneration_v1.0

| | Rel. Exp. (%) | | Rel. Exp. (%) |
|---|---|---|---|
| | Ag4348, Run | | Ag4348, Run |
| Tissue Name | 224364195 | Tissue Name | 224364195 |
| AD 1 Hippo | 14.2 | Control (Path) 3 | 6.3 |
| | | Temporal Ctx | |
| AD 2 Hippo | 30.4 | Control (Path) 4 | 39.8 |
| | | Temporal Ctx | |
| AD 3 Hippo | 10.3 | AD 1 Occipital Ctx | 21.9 |
| AD 4 Hippo | 16.7 | AD 2 Occipital Ctx | 0.0 |
| | | (Missing) | |
| AD 5 hippo | 100.0 | AD 3 Occipital Ctx | 9.2 |
| AD 6 Hippo | 49.0 | AD 4 Occipital Ctx | 23.3 |
| Control 2 Hippo | 21.5 | AD 5 Occipital Ctx | 25.3 |
| Control 4 Hippo | 12.1 | AD 6 Occipital Ctx | 29.5 |
| Control (Path) 3 Hippo | 5.1 | Control 1 Occipital | 3.8 |
| | | Ctx | |
| AD 1 Temporal Ctx | 19.8 | Control 2 Occipital | 36.9 |
| | | Ctx | |
| AD 2 Temporal Ctx | 28.1 | Control 3 Occipital | 29.7 |
| | | Ctx | |
| AD 3 Temporal Ctx | 8.3 | Control 4 Occipital | 6.3 |
| | | Ctx | |
| AD 4 Temporal Ctx | 22.8 | Control (Path) 1 | 65.5 |
| | | Occipital Ctx | |
| AD 5 Inf Temporal Ctx | 59.0 | Control (Path) 2 | 18.2 |
| | | Occipital Ctx | |
| AD 5 SupTemporal Ctx | 48.3 | Control (Path) 3 | 2.1 |
| | | Occipital Ctx | |
| AD 6 Inf Temporal Ctx | 51.1 | Control (Path) 4 | 37.9 |
| | | Occipital Ctx | |
| AD 6 Sup Temporal Ctx | 51.8 | Control 1 Parietal Ctx | 8.2 |
| Control 1 Temporal Ctx | 6.7 | Control 2 Parietal Ctx | 53.6 |
| Control 2 Temporal Ctx | 34.2 | Control 3 Parietal Ctx | 26.2 |
| Control 3 Temporal Ctx | 26.8 | Control (Path) 1 | 56.3 |
| | | Parietal Ctx | |
| Control 4 Temporal Ctx | 17.0 | Control (Path) 2 | 33.7 |
| | | Parietal Ctx | |
| Control (Path) 1 | 62.0 | Control (Path) 3 | 6.0 |
| Temporal Ctx | | Parietal Ctx | |
| Control (Path) 2 | 52.9 | Control (Path) 4 | 49.7 |
| Temporal Ctx | | Parietal Ctx | |

TABLE RE
General_screening_panel_v1.6

| | Rel. Exp. (%) | | Rel. Exp. (%) |
|---|---|---|---|
| | Ag6953, Run | | Ag6953, Run |
| Tissue Name | 278388895 | Tissue Name | 278388895 |
| Adipose | 8.2 | Renal ca. TK-10 | 0.0 |
| Melanoma* | 0.0 | Bladder | 30.8 |
| Hs688(A).T | | | |
| Melanoma* | 0.0 | Gastric ca. (liver met.) | 55.1 |
| Hs688(B).T | | NCI-N87 | |
| Melanoma* M14 | 0.0 | Gastric ca. KATO III | 0.0 |
| Melanoma* LOXIMVI | 0.0 | Colon ca. SW-948 | 0.0 |
| Melanoma* SK-MEL-5 | 9.3 | Colon ca. SW480 | 0.0 |
| Squamous cell | 26.6 | Colon ca.* (SW480 met) | 0.0 |
| carcinoma SCC-4 | | SW620 | |
| Testis Pool | 100.0 | Colon ca. HT29 | 0.0 |
| Prostate ca.* (bone met) | 9.7 | Colon ca. HCT-116 | 0.0 |
| PC-3 | | | |
| Prostate Pool | 17.7 | Colon ca. CaCo-2 | 10.0 |
| Placenta | 0.0 | Colon cancer tissue | 0.0 |
| Uterus Pool | 3.1 | Colon ca. SW1116 | 9.7 |
| Ovarian ca. OVCAR-3 | 0.0 | Colon ca. Colo-205 | 0.0 |
| Ovarian ca. SK-OV-3 | 10.1 | Colon ca. SW-48 | 0.0 |
| Ovarian ca. OVCAR-4 | 0.0 | Colon Pool | 40.6 |
| Ovarian ca. OVCAR-5 | 24.8 | Small Intestine Pool | 44.1 |
| Ovarian ca. IGROV-1 | 0.0 | Stomach Pool | 0.0 |
| Ovarian ca. OVCAR-8 | 0.0 | Bone Marrow Pool | 16.7 |
| Ovary | 10.3 | Fetal Heart | 10.2 |
| Breast ca. MCF-7 | 0.0 | Heart Pool | 8.1 |
| Breast ca. MDA-MB- | 92.0 | Lymph Node Pool | 47.3 |
| 231 | | | |
| Breast ca. BT 549 | 0.0 | Fetal Skeletal Muscle | 35.4 |
| Breast ca. T47D | 0.0 | Skeletal Muscle Pool | 0.0 |
| Breast ca. MDA-N | 0.0 | Spleen Pool | 0.0 |
| Breast Pool | 19.5 | Thymus pool | 48.3 |
| Trachea | 0.0 | CNS cancer (glio/astro) | 10.1 |
| | | U87-MG | |
| Lung | 0.0 | CNS cancer (glio/astro) U- | 0.0 |
| | | 118-MG | |
| Fetal Lung | 7.7 | CNS cancer (neuro; met) | 2.8 |
| | | SK-N-AS | |
| Lung ca. NCI-N417 | 0.0 | CNS cancer (astro) SF-539 | 0.0 |
| Lung ca. LX-1 | 8.3 | CNS cancer (astro) SNB- | 0.0 |
| | | 75 | |
| Lung ca. NCI-H146 | 0.0 | CNS cancer (glio) SNB-19 | 0.0 |
| Lung ca. SHP-77 | 0.0 | CNS cancer (glio) SF-295 | 12.1 |
| Lung ca. A549 | 0.0 | Brain (Amygdala) Pool | 0.0 |
| Lung ca. NCI-H526 | 0.0 | Brain (cerebellum) | 0.0 |
| Lung ca. NCI-H23 | 0.0 | Brain (fetal) | 9.5 |
| Lung ca. NCI-H460 | 0.0 | Brain (Hippocampus) Pool | 0.0 |
| Lung ca. HOP-62 | 0.0 | Cerebral Cortex Pool | 0.0 |
| Lung ca. NCI-H522 | 0.0 | Brain (Substantia nigra) | 7.3 |
| | | Pool | |
| Liver | 0.0 | Brain (Thalamus) Pool | 8.6 |
| Fetal Liver | 0.0 | Brain (whole) | 0.0 |
| Liver ca. HepG2 | 0.0 | Spinal Cord Pool | 0.0 |
| Kidney Pool | 13.7 | Adrenal Gland | 0.0 |
| Fetal Kidney | 8.1 | Pituitary gland Pool | 0.0 |
| Renal ca. 786-0 | 0.0 | Salivary Gland | 0.0 |
| Renal ca. A498 | 0.0 | Thyroid (female) | 0.0 |
| Renal ca. ACHN | 0.0 | Pancreatic ca. CAPAN2 | 37.9 |
| Renal ca. UO-31 | 0.0 | Pancreas Pool | 0.0 |

TABLE RF
Panel 4.1D

| | Rel. Exp. (%) | | Rel. Exp. (%) |
|---|---|---|---|
| | Ag4348, Run | | Ag4348, Run |
| Tissue Name | 186362432 | Tissue Name | 186362432 |
| Secondary Th1 act | 0.0 | HUVEC IL-1beta | 0.0 |
| Secondary Th2 act | 0.0 | HUVEC IFN gamma | 0.0 |
| Secondary Tr1 act | 1.6 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC | 0.0 |
| | | TNF alpha + IL-1beta | |
| Primary Th2 act | 0.0 | Microvascular Dermal EC | 0.0 |
| | | none | |
| Primary Tr1 act | 0.0 | Microsvasular Dermal EC | 0.0 |
| | | TNF alpha + IL-1beta | |
| Primary Th1 rest | 0.0 | Bronchial epithelium | 0.0 |
| | | TNF alpha + IL1beta | |
| Primary Th2 rest | 0.0 | Small airway epithelium none | 0.0 |
| Primary Tr1 rest | 1.5 | Small airway epithelium | 0.0 |
| | | TNF alpha + IL-1beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNF alpha + IL-1beta | |
| CD8 lymphocyte act | 0.0 | Astrocytes rest | 0.0 |
| Secondary CD8 | 0.0 | Astrocytes TNF alpha + IL- | 0.0 |
| lymphocyte rest | | 1beta | |
| Secondary CD8 | 0.0 | KU-812 (Basophil) rest | 0.0 |
| lymphocyte act | | | |
| CD4 lymphocyte none | 1.7 | KU-812 (Basophil) | 0.0 |
| | | PMA/ionomycin | |
| 2ry Th1/Th2/Tr1_anti- | 2.3 | CCD1106 (Keratinocytes) | 0.0 |
| CD95 CH11 | | none | |
| LAK cells rest | 0.0 | CCD1106 (Keratinocytes) | 0.0 |
| | | TNF alpha + IL-1beta | |
| LAK cells IL-2 | 0.0 | Liver cirrhosis | 0.0 |
| LAK cells IL-2 + IL-12 | 0.0 | NCI-H292 none | 0.0 |
| LAK cells IL-2 + IFN | 0.0 | NCI-H292 IL-4 | 0.0 |
| gamma | | | |
| LAK cells IL-2 + IL-18 | 0.0 | NCI-H292 IL-9 | 0.0 |
| LAK cells | 0.0 | NCI-H292 IL-13 | 0.0 |
| PMA/ionomycin | | | |
| NK Cells IL-2 rest | 0.0 | NCI-H292 IFN gamma | 0.0 |
| Two Way MLR 3 day | 0.0 | HPAEC none | 1.7 |
| Two Way MLR 5 day | 0.0 | HPAEC TNF alpha + IL-1beta | 0.0 |
| Two Way MLR 7 day | 0.0 | Lung fibroblast none | 0.0 |
| PBMC rest | 0.0 | Lung fibroblast TNF alpha + | 0.0 |
| | | IL-1beta | |
| PBMC PWM | 0.0 | Lung fibroblast IL-4 | 0.0 |
| PBMC PHA-L | 0.0 | Lung fibroblast IL-9 | 0.0 |
| Ramos (B cell) none | 0.0 | Lung fibroblast IL-13 | 0.0 |
| Ramos (B cell) ionomycin | 0.0 | Lung fibroblast IFN gamma | 0.0 |
| B lymphocytes PWM | 0.0 | Dermal fibroblast CCD1070 | 0.0 |
| | | rest | |
| B lymphocytes CD40L | 0.0 | Dermal fibroblast CCD1070 | 0.0 |
| and IL-4 | | TNF alpha | |
| EOL-1 dbcAMP | 0.0 | Dermal fibroblast CCD1070 | 0.0 |
| | | IL-1beta | |
| EOL-1 dbcAMP | 0.0 | Dermal fibroblast IFN gamma | 0.0 |
| PMA/ionomycin | | | |
| Dendritic cells none | 0.0 | Dermal fibroblast IL-4 | 0.0 |
| Dendritic cells LPS | 0.0 | Dermal fibroblast rest | 2.0 |
| Dendritic cells anti-CD40 | 0.0 | Neutrophils TNFa + LPS | 3.8 |
| Monocytes rest | 0.0 | Neutrophils rest | 0.0 |
| Monocytes LPS | 0.0 | Colon | 3.5 |
| Macrophages rest | 0.0 | Lung | 6.8 |
| Macrophages LPS | 0.0 | Thymus | 7.2 |
| HUVEC none | 0.0 | Kidney | 100.0 |
| HUVEC starved | 1.8 | | |

CNS_neurodegeneration_v1.0 Summary: Ag4348 This panel confirms the expression of the CG105491-01 gene at low levels in the brain in an independent group of individuals. This gene is found to be slightly down-regulated in the temporal cortex of Alzheimer's disease patients. Therefore, up-regulation of this gene or its protein product, or treatment with specific agonists for this protein may be of use in reversing the dementia/memory loss associated with this disease and neuronal death.

The CG105491-01 gene codes for a serine protease. Plasmin, a member of serine protease family, is shown to increase the processing of human APP preferentially at the alpha-cleavage site, and efficiently degrades secreted amyloidogenic and non-amyloidogenic APP fragments. Brain tissue from Alzheimer's disease patients was shown to contain reduced levels of plasmin, implying that plasmin downregulation may cause amyloid plaque deposition accompanying sporadic Alzheimer's disease (Ledesma et al., 2000, EMBO Rep 1(6):530-5, PMID: 11263499). Thus, based on functional homology and also, on expression pattern, the serine protease encoded by this gene may also play a role in degradation of amyloidogenic and non-amyloidogenic APP fragments. Therefore, therapeutic modulation of this gene product may be beneficial in the treatment of Alzheimer's disease.

General_screening_panel_v1.6 Summary: Ag6953 Highest expression of this gene is detected in testis and a breast cancer MDA-MB-231 cell line (CTs=33.1). Therefore, expression of this gene may be used to distinguish these samples from other samples in this panel. In addition, low expression of this gene is detected in pancreatic, a gastric, and squamous cell cancer cell lines. Therefore, expression of this gene may be used as diagnostic marker for detection of squamous cell carcinoma, breast, pancreatic, and gastric cancers. In additon, therapeutic modulation of this gene product may be useful in the treatment of these cancers.

In addition to testis, low levels of expression of this gene is also seen in normal tissues represented by thymus, lymphnode, bladder, colon and small intestine. Therefore, therapeutic modulation of this gene may be useful in the treatment of disease associated with these tissues.

Low levels of expression of this gene is also detected in fetal skeletal muscle. Interestingly, this gene is expressed at much higher levels in fetal (CT=34.5) when compared to adult skeletal muscle (CT=40). This observation suggests that expression of this gene can be used to distinguish fetal from adult skeletal muscle. In addition, the relative overexpression of this gene in fetal skeletal muscle suggests that the protein product may enhance muscular growth or development in the fetus and thus may also act in a regenerative capacity in the adult. Therefore, therapeutic modulation of the protein encoded by this gene could be useful in treatment of muscle related diseases. More specifically, treatment of weak or dystrophic muscle with the protein encoded by this gene could restore muscle mass or function.

Panel 4.1D Summary: Ag4348 Moderate level of expression of the CG105491-01 gene is detected only in kidney sample (CT=31.2). Therefore, expression of this gene may be used to distinguish kidney from other samples used in this panel. In addition, therapeutic modulation of this gene product may be beneficial in the treatment of autoimmune and inflammatory diseases that affect kidney, including lupus and glomerulonephritis.

S. CG105954-01: Human Ortholog of Chicken NEUROFASCIN PRECURSOR

Expression of gene CG105954-01 was assessed using the primer-probe set Ag4311, described in Table SA.

TABLE SA

Probe Name Ag4311

| | | | Start | SEQ ID |
|---|---|---|---|---|
| Primers | Sequences | Length | Position | No |
| Forward | 5′-aatgggatcatgattggataca-3′ | 22 | 2890 | 210 |
| Probe | TET-5′-aatatgtggcctgtacgttctcccca-3′-TAMRA | 26 | 2918 | 211 |
| Reverse | 5′-ttcctactttggtcccgttaac-3′ | 22 | 2944 | 212 |

T. CG105963-01: Novel Cadherin

Expression of gene CG105963-01 was assessed using the primer-probe set Ag4312, described in Table TA. Results of the RTQ-PCR runs are shown in Tables TB and TC.

TABLE TA

Probe Name Ag4312

| | | | Start | SEQ ID |
|---|---|---|---|---|
| Primers | Sequences | Length | Position | No |
| Forward | 5′-cagccctcatctatgactacga-3′ | 22 | 2219 | 213 |
| Probe | TET-5′-acgctgagctccatcctgtccag-3′-TAMRA | 23 | 2263 | 214 |
| Reverse | 5′-agtcgtagtcctggtcctcatc-3′ | 22 | 2293 | 215 |

TABLE TB
General_screening_panel_v1.4

| | Rel. Exp. (%) | | Rel. Exp. (%) |
|---|---|---|---|
| | Ag4312, Run | | Ag4312, Run |
| Tissue Name | 222355477 | Tissue Name | 222355477 |
| Adipose | 1.4 | Renal ca. TK-10 | 7.3 |
| Melanoma* | 1.2 | Bladder | 0.3 |
| Hs688(A).T | | | |
| Melanoma* | 1.2 | Gastric ca. (liver met.) | 2.1 |
| Hs688(B).T | | NCI-N87 | |
| Melanoma* M14 | 0.0 | Gastric ca. KATO III | 0.0 |
| Melanoma* LOXIMVI | 0.7 | Colon ca. SW-948 | 0.0 |
| Melanoma* SK-MEL-5 | 0.0 | Colon ca. SW480 | 1.4 |
| Squamous cell | 0.2 | Colon ca.* (SW480 met) | 0.5 |
| carcinoma SCC-4 | | SW620 | |
| Testis Pool | 0.3 | Colon ca. HT29 | 0.0 |
| Prostate ca.* (bone met) | 13.8 | Colon ca. HCT-116 | 8.1 |
| PC-3 | | | |
| Prostate Pool | 0.6 | Colon ca. CaCo-2 | 0.0 |
| Placenta | 0.3 | Colon cancer tissue | 0.5 |
| Uterus Pool | 0.0 | Colon ca. SW1116 | 1.3 |
| Ovarian ca. OVCAR-3 | 0.5 | Colon ca. Colo-205 | 0.0 |
| Ovarian ca. SK-OV-3 | 0.4 | Colon ca. SW-48 | 0.0 |
| Ovarian ca. OVCAR-4 | 1.3 | Colon Pool | 0.4 |
| Ovarian ca. OVCAR-5 | 9.0 | Small Intestine Pool | 0.0 |
| Ovarian ca. IGROV-1 | 3.4 | Stomach Pool | 0.0 |
| Ovarian ca. OVCAR-8 | 7.7 | Bone Marrow Pool | 0.0 |
| Ovary | 0.0 | Fetal Heart | 0.0 |
| Breast ca. MCF-7 | 1.0 | Heart Pool | 0.1 |
| Breast ca. MDA-MB- | 1.1 | Lymph Node Pool | 0.3 |
| 231 | | | |
| Breast ca. BT 549 | 0.6 | Fetal Skeletal Muscle | 38.7 |
| Breast ca. T47D | 17.4 | Skeletal Muscle Pool | 34.4 |
| Breast ca. MDA-N | 0.0 | Spleen Pool | 0.3 |
| Breast Pool | 0.0 | Thymus pool | 0.3 |
| Trachea | 0.2 | CNS cancer (glio/astro) | 9.6 |
| | | U87-MG | |
| Lung | 0.1 | CNS cancer (glio/astro) U- | 0.1 |
| | | 118-MG | |
| Fetal Lung | 1.3 | CNS cancer (neuro; met) | 0.0 |
| | | SK-N-AS | |
| Lung ca. NCI-N417 | 21.2 | CNS cancer (astro) SF-539 | 4.3 |
| Lung ca. LX-1 | 1.7 | CNS cancer (astro) SNB- | 14.4 |
| | | 75 | |
| Lung ca. NCI-H146 | 0.0 | CNS cancer (glio) SNB-19 | 3.8 |
| Lung ca. SHP-77 | 1.2 | CNS cancer (glio) SF-295 | 6.7 |
| Lung ca. A549 | 0.1 | Brain (Amygdala) Pool | 0.0 |
| Lung ca. NCI-H526 | 0.0 | Brain (cerebellum) | 100.0 |
| Lung ca. NCI-H23 | 0.2 | Brain (fetal) | 0.0 |
| Lung ca. NCI-H460 | 0.0 | Brain (Hippocampus) Pool | 0.0 |
| Lung ca. HOP-62 | 8.8 | Cerebral Cortex Pool | 0.0 |
| Lung ca. NCI-H522 | 0.7 | Brain (Substantia nigra) | 0.1 |
| | | Pool | |
| Liver | 0.5 | Brain (Thalamus) Pool | 0.1 |
| Fetal Liver | 0.5 | Brain (whole) | 9.7 |
| Liver ca. HepG2 | 18.9 | Spinal Cord Pool | 0.0 |
| Kidney Pool | 0.2 | Adrenal Gland | 0.6 |
| Fetal Kidney | 1.8 | Pituitary gland Pool | 0.0 |
| Renal ca. 786-0 | 0.2 | Salivary Gland | 0.1 |
| Renal ca. A498 | 0.6 | Thyroid (female) | 0.0 |
| Renal ca. ACHN | 0.0 | Pancreatic ca. CAPAN2 | 0.0 |
| Renal ca. UO-31 | 0.0 | Pancreas Pool | 0.2 |

TABLE TC
Panel 4.1D

| | Rel. Exp. (%) | | Rel. Exp. (%) |
|---|---|---|---|
| | Ag4312, Run | | Ag4312, Run |
| Tissue Name | 182243751 | Tissue Name | 182243751 |
| Secondary Th1 act | 0.0 | HUVEC IL-1beta | 0.3 |
| Secondary Th2 act | 0.0 | HUVEC IFN gamma | 0.3 |
| Secondary Tr1 act | 0.0 | HUVEC TNF alpha + IFN | 1.5 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 4.6 |
| Primary Th1 act | 0.0 | Lung Microvascular EC | 0.4 |
| | | TNF alpha + IL-1beta | |
| Primary Th2 act | 0.1 | Microvascular Dermal EC | 0.2 |
| | | none | |
| Primary Tr1 act | 0.0 | Microsvasular Dermal EC | 0.5 |
| | | TNF alpha + IL-1beta | |
| Primary Th1 rest | 0.0 | Bronchial epithelium | 0.0 |
| | | TNF alpha + IL1beta | |
| Primary Th2 rest | 0.0 | Small airway epithelium none | 0.0 |
| Primary Tr1 rest | 0.0 | Small airway epithelium | 0.0 |
| | | TNF alpha + IL-1beta | |
| CD45RA CD4 | 0.1 | Coronery artery SMC rest | 1.2 |
| lymphocyte act | | | |
| CD45RO CD4 | 0.0 | Coronery artery SMC | 2.1 |
| lymphocyte act | | TNF alpha + IL-1beta | |
| CD8 lymphocyte act | 0.0 | Astrocytes rest | 0.0 |
| Secondary CD8 | 0.0 | Astrocytes TNF alpha + IL- | 0.0 |
| lymphocyte rest | | 1beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.1 | CCD1106 (Keratinocytes) | 0.0 |
| CD95 CH11 | | none | |
| LAK cells rest | 0.0 | CCD1106 (Keratinocytes) | 0.0 |
| | | TNF alpha + IL-1beta | |
| LAK cells IL-2 | 0.0 | Liver cirrhosis | 0.0 |
| LAK cells IL-2 + IL-12 | 0.0 | NCI-H292 none | 0.0 |
| LAK cells IL-2 + IFN | 0.0 | NCI-H292 IL-4 | 0.3 |
| gamma | | | |
| LAK cells IL-2 + IL-18 | 0.0 | NCI-H292 IL-9 | 0.0 |
| LAK cells | 0.0 | NCI-H292 IL-13 | 0.0 |
| PMA/ionomycin | | | |
| NK Cells IL-2 rest | 0.0 | NCI-H292 IFN gamma | 0.0 |
| Two Way MLR 3 day | 0.0 | HPAEC none | 0.0 |
| Two Way MLR 5 day | 0.0 | HPAEC TNF alpha + IL-1beta | 2.4 |
| Two Way MLR 7 day | 0.0 | Lung fibroblast none | 0.5 |
| PBMC rest | 0.0 | Lung fibroblast TNF alpha + | 4.0 |
| | | IL-1beta | |
| PBMC PWM | 0.0 | Lung fibroblast IL-4 | 0.0 |
| PBMC PHA-L | 0.0 | Lung fibroblast IL-9 | 0.7 |
| Ramos (B cell) none | 0.0 | Lung fibroblast IL-13 | 1.3 |
| Ramos (B cell) ionomycin | 0.0 | Lung fibroblast IFN gamma | 0.0 |
| B lymphocytes PWM | 0.0 | Dermal fibroblast CCD1070 | 1.1 |
| | | rest | |
| B lymphocytes CD40L | 0.0 | Dermal fibroblast CCD1070 | 0.0 |
| and IL-4 | | TNF alpha | |
| EOL-1 dbcAMP | 0.1 | Dermal fibroblast CCD1070 | 0.1 |
| | | IL-1beta | |
| EOL-1 dbcAMP | 0.6 | Dermal fibroblast IFN gamma | 0.4 |
| PMA/ionomycin | | | |
| Dendritic cells none | 0.0 | Dermal fibroblast IL-4 | 0.0 |
| Dendritic cells LPS | 0.0 | Dermal Fibroblasts rest | 0.1 |
| Dendritic cells anti-CD40 | 0.0 | Neutrophils TNFa + LPS | 0.2 |
| Monocytes rest | 0.0 | Neutrophils rest | 0.7 |
| Monocytes LPS | 0.0 | Colon | 0.1 |
| Macrophages rest | 0.0 | Lung | 2.4 |
| Macrophages LPS | 0.0 | Thymus | 13.9 |
| HUVEC none | 0.1 | Kidney | 100.0 |
| HUVEC starved | 0.2 | | |

General_screening_panel_v1.4 Summary: Ag4312 Highest expression of the CG105963-01 gene is detected in brain (cerebellum) (Ct=28.8). In addition, moderate expression of this gene is also seen in whole brain sample. The CG105963-01 gene codes for a variant of cadherin-15 (M-cadherin). Cadherins are calcium-dependent, transmembrane intercellular adhesion proteins with morphoregulatory functions in the development and maintenance of tissues. Cadherins can act as axon guidance and cell adhesion proteins, specifically during development and in the response to injury (Ranscht B., 2000, Int. J. Dev. Neurosci. 18: 643-651, PMID: 10978842). In addition, M-cadherin is involved in muscle cell, Schwann cell, and motoneuron interactions and also in differentiation during neuromuscular development (Padilla et al., 1998, Mol Cell Neurosci 11(4):217-33, PMID: 9675053). Therefore, therapeutic modulation of this protein may be useful in inducing a compensatory synaptogenic response to neuronal death in Alzheimer's disease, Parkinson's disease, Huntington's disease, spinocerebellar ataxia, progressive supranuclear palsy, ALS, head trauma, stroke, or any other disease/condition associated with neuronal loss.

In addition, low to moderate levels of expression of this gene is also seen in number of cancer cell lines including CNS cancer, colon, renal, liver, lung, breast, ovarian and prostate cancer cell lines. Therefore, therapeutic modulation of this gene product may be useful in the treatment of these cancers.

Moderate expression of this gene is also seen in skeletal muscle. M-cadherin is shown to be important for skeletal muscle development, in particular the fusion of myoblasts into myotubes (Kaufmann et al., 1999, J Cell Sci 112:55-68, PMID: 9841904). Therefore, therapeutic modulation of this gene may be beneficial in the treatment of muscle related disease

Panel 4.1D Summary: Ag4312 Highest expression of the CG105963-01 gene is detected in kidney (CT=28.3). Therefore, expression of this gene may be used to distinguish kidney sample from other samples used in this panel. In addition, moderate to low expression of this gene is also seen in thymus, lung, TNF alpha+IL-1 beta treated lung fibroblasts and endothelial cells represent by HPAEC and HUVEC, coronery artery, and lung microvascular EC. Therefore, therapeutic modulation of this gene product may be beneficial in the treatment of autoimmune and inflammatory diseases affecting kidney and lung including lupus erythematosus, asthma, emphysema, Crohn's disease, ulcerative colitis, rheumatoid arthritis, osteoarthritis, and psoriasis.

U. CG105973-01 and CG105973-02: Integrin Alpha-8

Expression of gene CG105973-01 and CG105973-02 was assessed using the primer-probe sets Ag4305 and Ag4313, described in Tables UA and UB. Results of the RTQ-PCR runs are shown in Tables UC, UD, UE, UF and UG. Please note that CG105973-02 represents a full-length physical clone of the CG105973-01 gene, validating the prediction of the gene sequence.

TABLE UA

Probe Name Ag4305

| | | | Start | SEQ ID |
|---|---|---|---|---|
| Primers | Sequences | Length | Position | No |
| Forward | 5′-agttccacgtcttgagaaaaca-3′ | 22 | 2589 | 216 |
| Probe | TET-5′-tgagcattaacttcgatctccaaatca-3′-TAMRA | 27 | 2615 | 217 |
| Reverse | 5′-gctgtctggattgtccttgtt-3′ | 21 | 2650 | 218 |

TABLE UB

Probe Name Ag4313

| | | | Start | SEQ ID |
|---|---|---|---|---|
| Primers | Sequences | Length | Position | No |
| Forward | 5′-agttccacgtcttgagaaaaca-3′ | 22 | 2589 | 219 |
| Probe | TET-5′-tgagcattaacttcgatctccaaatca-3′-TAMRA | 27 | 2615 | 220 |
| Reverse | 5′-gctgtctggattgtccttgtt-3′ | 21 | 2650 | 221 |

TABLE UC
AI_comprehensive panel_v1.0

| | Rel. Exp. (%) | | Rel. Exp. (%) |
|---|---|---|---|
| | Ag4305, Run | | Ag4305, Run |
| Tissue Name | 244570379 | Tissue Name | 244570379 |
| 110967 COPD-F | 17.0 | 112427 Match Control | 37.1 |
| | | Psoriasis-F | |
| 110980 COPD-F | 59.5 | 112418 Psoriasis-M | 14.5 |
| 110968 COPD-M | 15.7 | 112723 Match Control | 0.0 |
| | | Psoriasis-M | |
| 110977 COPD-M | 76.8 | 112419 Psoriasis-M | 33.0 |
| 110989 Emphysema-F | 22.8 | 112424 Match Control | 15.6 |
| | | Psoriasis-M | |
| 110992 Emphysema-F | 4.6 | 112420 Psoriasis-M | 27.2 |
| 110993 Emphysema-F | 21.6 | 112425 Match Control | 28.1 |
| | | Psoriasis-M | |
| 110994 Emphysema-F | 14.3 | 104689 (MF) OA Bone- | 17.2 |
| | | Backus | |
| 110995 Emphysema-F | 10.2 | 104690 (MF) Adj “Normal” | 18.7 |
| | | Bone-Backus | |
| 110996 Emphysema-F | 0.0 | 104691 (MF) OA | 10.4 |
| | | Synovium-Backus | |
| 110997 Asthma-M | 13.9 | 104692 (BA) OA Cartilage- | 0.0 |
| | | Backus | |
| 111001 Asthma-F | 17.1 | 104694 (BA) OA Bone- | 6.3 |
| | | Backus | |
| 111002 Asthma-F | 14.8 | 104695 (BA) Adj “Normal” | 16.8 |
| | | Bone-Backus | |
| 111003 Atopic Asthma-F | 23.0 | 104696 (BA) OA | 6.0 |
| | | Synovium-Backus | |
| 111004 Atopic Asthma-F | 17.1 | 104700 (SS) OA Bone- | 11.8 |
| | | Backus | |
| 111005 Atopic Asthma-F | 12.8 | 104701 (SS) Adj “Normal” | 10.2 |
| | | Bone-Backus | |
| 111006 Atopic Asthma-F | 5.1 | 104702 (SS) OA | 23.3 |
| | | Synovium-Backus | |
| 111417 Allergy-M | 13.6 | 117093 OA Cartilage Rep7 | 18.8 |
| 112347 Allergy-M | 7.1 | 112672 OA Bone5 | 36.9 |
| 112349 Normal Lung-F | 5.7 | 112673 OA Synovium5 | 17.6 |
| 112357 Normal Lung-F | 12.9 | 112674 OA Synovial Fluid | 17.0 |
| | | cells5 | |
| 112354 Normal Lung-M | 59.9 | 117100 OA Cartilage | 5.0 |
| | | Rep14 | |
| 112374 Crohns-F | 8.0 | 112756 OA Bone9 | 0.2 |
| 112389 Match Control | 24.3 | 112757 OA Synovium9 | 3.3 |
| Crohns-F | | | |
| 112375 Crohns-F | 14.3 | 112758 OA Synovial Fluid | 11.0 |
| | | Cells9 | |
| 112732 Match Control | 6.2 | 117125 RA Cartilage Rep2 | 19.1 |
| Crohns-F | | | |
| 112725 Crohns-M | 11.3 | 113492 Bone2 RA | 40.9 |
| 112387 Match Control | 5.9 | 113493 Synovium2 RA | 21.6 |
| Crohns-M | | | |
| 112378 Crohns-M | 7.5 | 113494 Syn Fluid Cells RA | 34.4 |
| 112390 Match Control | 32.3 | 113499 Cartilage4 RA | 21.3 |
| Crohns-M | | | |
| 112726 Crohns-M | 100.0 | 113500 Bone4 RA | 26.8 |
| 112731 Match Control | 47.0 | 113501 Synovium4 RA | 24.3 |
| Crohns-M | | | |
| 112380 Ulcer Col-F | 17.1 | 113502 Syn Fluid Cells4 | 13.5 |
| | | RA | |
| 112734 Match Control | 14.3 | 113495 Cartilage3 RA | 28.3 |
| Ulcer Col-F | | | |
| 112384 Ulcer Col-F | 17.6 | 113496 Bone3 RA | 33.0 |
| 112737 Match Control | 35.1 | 113497 Synovium3 RA | 15.7 |
| Ulcer Col-F | | | |
| 112386 Ulcer Col-F | 3.0 | 113498 Syn Fluid Cells3 | 39.2 |
| | | RA | |
| 112738 Match Control | 6.7 | 117106 Normal Cartilage | 4.3 |
| Ulcer Col-F | | Rep20 | |
| 112381 Ulcer Col-M | 33.2 | 113663 Bone3 Normal | 12.7 |
| 112735 Match Control | 40.9 | 113664 Synovium3 Normal | 1.5 |
| Ulcer Col-M | | | |
| 112382 Ulcer Col-M | 37.9 | 113665 Syn Fluid Cells3 | 7.5 |
| | | Normal | |
| 112394 Match Control | 0.2 | 117107 Normal Cartilage | 10.0 |
| Ulcer Col-M | | Rep22 | |
| 112383 Ulcer Col-M | 3.5 | 113667 Bone4 Normal | 13.5 |
| 112736 Match Control | 19.2 | 113668 Synovium4 Normal | 16.3 |
| Ulcer Col-M | | | |
| 112423 Psoriasis-F | 39.2 | 113669 Syn Fluid Cells4 | 20.2 |
| | | Normal | |

TABLE UD
CNS_neurodegeneration_v1.0

| | Rel. Exp. (%) | Rel. Exp. (%) | | Rel. Exp. (%) | Rel. Exp. (%) |
|---|---|---|---|---|---|
| | Ag4305, Run | Ag4313, Run | | Ag4305, Run | Ag4313, Run |
| Tissue Name | 224074285 | 224064613 | Tissue Name | 224074285 | 224064613 |
| AD 1 Hippo | 24.0 | 29.1 | Control (Path) | 17.0 | 28.5 |
| | | | 3 Temporal | | |
| | | | Ctx | | |
| AD 2 Hippo | 36.9 | 39.0 | Control (Path) | 37.1 | 39.0 |
| | | | 4 Temporal | | |
| | | | Ctx | | |
| AD 3 Hippo | 25.9 | 34.2 | AD 1 Occipital | 20.4 | 18.4 |
| | | | Ctx | | |
| AD 4 Hippo | 9.2 | 9.4 | AD 2 Occipital | 0.0 | 0.0 |
| | | | Ctx (Missing) | | |
| AD 5 Hippo | 55.9 | 76.8 | AD 3 Occipital | 15.2 | 9.9 |
| | | | Ctx | | |
| AD 6 Hippo | 100.0 | 100.0 | AD 4 Occipital | 20.9 | 25.9 |
| | | | Ctx | | |
| Control 2 | 37.6 | 32.3 | AD 5 Occipital | 50.7 | 22.7 |
| Hippo | | | Ctx | | |
| Control 4 | 27.9 | 25.7 | AD 6 Occipital | 32.1 | 59.5 |
| Hippo | | | Ctx | | |
| Control (Path) | 44.4 | 54.7 | Control 1 | 9.6 | 13.2 |
| 3 Hippo | | | Occipital Ctx | | |
| AD 1 | 30.8 | 25.2 | Control 2 | 32.3 | 40.9 |
| Temporal Ctx | | | Occipital Ctx | | |
| AD 2 | 43.5 | 52.1 | Control 3 | 25.0 | 26.2 |
| Temporal Ctx | | | Occipital Ctx | | |
| AD 3 | 15.3 | 20.4 | Control 4 | 14.9 | 27.5 |
| Temporal Ctx | | | Occiptial Ctx | | |
| AD 4 | 28.3 | 37.4 | Control (Path) | 47.3 | 64.2 |
| Temporal Ctx | | | 1 Occipital Ctx | | |
| AD 5 Inf | 66.0 | 70.7 | Control (Path) | 12.7 | 18.0 |
| Temporal Ctx | | | 2 Occipital Ctx | | |
| AD 5 Sup | 73.2 | 69.7 | Control (Path) | 9.0 | 12.8 |
| Temporal Ctx | | | 3 Occipital Ctx | | |
| AD 6 Inf | 40.3 | 43.5 | Control (Path) | 25.2 | 37.1 |
| Temporal Ctx | | | 4 Occipital Ctx | | |
| AD 6 Sup | 62.9 | 62.4 | Control 1 | 14.3 | 13.1 |
| Temporal Ctx | | | Parietal Ctx | | |
| Control 1 | 13.4 | 20.0 | Control 2 | 60.7 | 51.8 |
| Temporal Ctx | | | Parietal Ctx | | |
| Control 2 | 28.5 | 27.4 | Control 3 | 40.9 | 33.7 |
| Temporal Ctx | | | Parietal Ctx | | |
| Control 3 | 25.3 | 22.4 | Control (Path) | 42.0 | 62.9 |
| Temporal Ctx | | | 1 Parietal Ctx | | |
| Control 3 | 24.7 | 20.2 | Control (Path) | 42.6 | 38.2 |
| Temporal Ctx | | | 2 Parietal Ctx | | |
| Control (Path) | 54.7 | 66.0 | Control (Path) | 21.6 | 19.6 |
| 1 Temporal | | | 3 Parietal Ctx | | |
| Ctx | | | | | |
| Control (Path) | 36.6 | 33.9 | Control (Path) | 59.5 | 77.9 |
| 2 Temporal | | | 4 Parietal Ctx | | |
| Ctx | | | | | |

TABLE UE
General_screening_panel_v1.4

| | Rel. Exp. (%) | Rel. Exp. (%) | | Rel. Exp. (%) | Rel. Exp. (%) |
|---|---|---|---|---|---|
| | Ag4305, Run | Ag4313, Run | | Ag4305, Run | Ag4313, Run |
| Tissue Name | 222261511 | 222360603 | Tissue Name | 222261511 | 222360603 |
| Adipose | 8.7 | 9.5 | Renal ca. TK-10 | 0.0 | 0.0 |
| Melanoma* | 19.8 | 20.0 | Bladder | 2.7 | 3.0 |
| Hs688(A).T | | | | | |
| Melanoma* | 18.0 | 15.0 | Gastric ca. (liver | 0.0 | 0.0 |
| Hs688(B).T | | | met.) NCI-N87 | | |
| Melanoma* | 0.0 | 0.0 | Gastric ca. KATO | 0.0 | 0.0 |
| M14 | | | III | | |
| Melanoma* | 0.0 | 0.0 | Colon ca. SW-948 | 0.0 | 0.0 |
| LOXIMVI | | | | | |
| Melanoma* SK- | 0.0 | 0.0 | Colon ca. SW480 | 0.0 | 0.0 |
| MEL-5 | | | | | |
| Squamous cell | 0.0 | 0.0 | Colon ca.* (SW480 | 0.0 | 0.0 |
| carcinoma SCC-4 | | | met) SW620 | | |
| Testis Pool | 10.8 | 8.9 | Colon ca. HT29 | 0.0 | 0.0 |
| Prostate ca.* | 0.0 | 0.0 | Colon ca. HCT-116 | 0.0 | 0.0 |
| (bone met) PC-3 | | | | | |
| Prostate Pool | 27.9 | 24.0 | Colon ca. CaCo-2 | 0.0 | 0.0 |
| Placenta | 0.0 | 0.0 | Colon cancer tissue | 2.2 | 1.7 |
| Uterus Pool | 13.2 | 10.4 | Colon ca. SW1116 | 0.0 | 0.0 |
| Ovarian ca. | 0.0 | 0.0 | Colon ca. Colo-205 | 0.0 | 0.0 |
| OVCAR-3 | | | | | |
| Ovarian ca. SK- | 0.0 | 0.0 | Colon ca. SW-48 | 0.0 | 0.0 |
| OV-3 | | | | | |
| Ovarian ca. | 0.0 | 0.0 | Colon Pool | 18.3 | 19.8 |
| OVCAR-4 | | | | | |
| Ovarian ca. | 0.0 | 0.0 | Small Intestine | 13.9 | 11.7 |
| OVCAR-5 | | | Pool | | |
| Ovarian ca. | 0.0 | 0.0 | Stomach Pool | 10.0 | 10.6 |
| IGROV-1 | | | | | |
| Ovarian ca. | 0.0 | 0.0 | Bone Marrow Pool | 20.9 | 20.2 |
| OVACAR-8 | | | | | |
| Ovary | 1.9 | 1.3 | Fetal Heart | 3.7 | 2.7 |
| Breast ca. MCF-7 | 0.0 | 0.0 | Heart Pool | 17.0 | 15.4 |
| Breast ca. | 0.0 | 0.0 | Lymph Node Pool | 29.5 | 24.1 |
| MDA-MB-231 | | | | | |
| Breast ca. BT | 0.0 | 0.0 | Fetal Skeletal | 5.6 | 4.2 |
| 549 | | | Muscle | | |
| Breast ca. T47D | 0.0 | 0.0 | Skeletal Muscle | 5.7 | 4.7 |
| | | | Pool | | |
| Breast ca. | 0.0 | 0.0 | Spleen Pool | 37.4 | 31.2 |
| MDA-N | | | | | |
| Breast Pool | 20.9 | 17.6 | Thymus Pool | 8.2 | 7.6 |
| Trachea | 14.1 | 12.7 | CNS cancer | 0.1 | 0.0 |
| | | | (glio/astro) U87- | | |
| | | | MG | | |
| Lung | 21.5 | 18.6 | CNS cancer | 28.9 | 25.5 |
| | | | (glio/astro) U-118- | | |
| | | | MG | | |
| Fetal Lung | 100.0 | 100.0 | CNS cancer | 83.5 | 69.7 |
| | | | (neuro;met) SK-N- | | |
| | | | AS | | |
| Lung ca. NCI- | 0.0 | 0.0 | CNS cancer (astro) | 0.3 | 0.0 |
| N417 | | | SF-539 | | |
| Lung ca. LX-1 | 0.0 | 0.0 | CNS cancer (astro) | 0.3 | 0.0 |
| | | | SNB-75 | | |
| Lung ca. NCI- | 0.0 | 0.0 | CNS cancer (glio) | 0.0 | 0.0 |
| H146 | | | SNB-19 | | |
| Lung ca. SHP- | 0.0 | 0.0 | CNS cancer (glio) | 40.6 | 35.1 |
| 77 | | | SF-295 | | |
| Lung ca. A549 | 0.0 | 0.0 | Brain (Amygdala) | 2.2 | 2.3 |
| | | | Pool | | |
| Lung ca. NCI- | 0.0 | 0.0 | Brain (cerebellum) | 5.2 | 3.7 |
| H526 | | | | | |
| Lung ca. NCI- | 0.5 | 0.1 | Brain (fetal) | 3.2 | 3.9 |
| H23 | | | | | |
| Lung ca. NCI- | 0.0 | 0.0 | Brain | 6.4 | 5.0 |
| H460 | | | (Hippocampus) | | |
| | | | Pool | | |
| Lung ca. HOP- | 0.0 | 0.0 | Cerebral Cortex | 3.6 | 3.2 |
| 62 | | | Pool | | |
| Lung ca. NCI- | 0.0 | 0.0 | Brain (Substantia | 2.2 | 2.4 |
| H522 | | | nigra) Pool | | |
| Liver | 0.1 | 0.0 | Brain (Thalamus) | 5.7 | 5.9 |
| | | | Pool | | |
| Fetal Liver | 3.7 | 3.0 | Brain (whole) | 2.5 | 2.6 |
| Liver ca. | 0.0 | 0.0 | Spinal Cord Pool | 3.6 | 2.3 |
| HepG2 | | | | | |
| Kidney Pool | 52.9 | 42.0 | Adrenal Gland | 17.0 | 21.2 |
| Fetal Kidney | 45.1 | 40.3 | Pitutitary gland | 3.7 | 2.7 |
| | | | Pool | | |
| Renal ca. 786-0 | 0.0 | 0.0 | Salivary Gland | 1.3 | 0.2 |
| Renal ca. A498 | 0.0 | 0.0 | Thyroid (female) | 2.9 | 2.7 |
| Renal ca. | 0.0 | 0.0 | Pancreatic ca. | 0.0 | 0.0 |
| ACHN | | | CAPAN2 | | |
| Renal ca. UO- | 0.0 | 0.0 | Pancreas Pool | 8.4 | 8.9 |
| 31 | | | | | |

TABLE UF
Panel 4.1D

| | Rel. Exp. (%) | Rel. Exp. (%) | | Rel. Exp. (%) | Rel. Exp. (%) |
|---|---|---|---|---|---|
| | Ag4305, Run | Ag4313, Run | | Ag4305, Run | Ag4313, Run |
| Tissue Name | 182086760 | 182244195 | Tissue Name | 182086760 | 182244195 |
| Secondary Th1 act | 0.0 | 0.0 | HUVEC IL-1beta | 0.6 | 0.0 |
| Secondary Th2 act | 0.0 | 0.0 | HUVEC IFN gamma | 0.0 | 0.0 |
| Secondary Tr1 act | 0.0 | 0.0 | HUVEC TNF alpha + | 0.0 | 0.0 |
| | | | IFN gamma | | |
| Secondary Th1 rest | 0.0 | 0.0 | HUVEC TNF alpha + | 0.0 | 0.0 |
| | | | IL4 | | |
| Secondary Th2 rest | 0.0 | 0.0 | HUVEC IL-11 | 0.0 | 0.0 |
| Secondary Tr1 rest | 0.0 | 0.0 | Lung Microvascular | 0.3 | 0.0 |
| | | | EC none | | |
| Primary Th1 act | 0.0 | 0.0 | Lung Microvascular | 3.9 | 0.0 |
| | | | EC TNF alpha + IL- | | |
| | | | 1beta | | |
| Primary Th2 act | 0.0 | 0.0 | Microvascular | 0.0 | 0.0 |
| | | | Dermal EC none | | |
| Primary Tr1 act | 0.0 | 0.0 | Microsvasular | 0.0 | 1.0 |
| | | | Dermal EC | | |
| | | | TNF alpha + IL-1beta | | |
| Primary Th1 rest | 0.0 | 0.0 | Bronchial epithelium | 0.0 | 0.0 |
| | | | TNF alpha + IL1beta | | |
| Primary Th2 rest | 0.0 | 0.0 | Small airway | 0.0 | 0.0 |
| | | | epithelium none | | |
| Primary Tr1 rest | 0.0 | 0.0 | Small airway | 0.0 | 0.0 |
| | | | epithelium TNF alpha + | | |
| | | | IL-1beta | | |
| CD45RA CD4 | 3.8 | 8.7 | Coronery artery | 0.0 | 0.0 |
| lymphocyte act | | | SMS rest | | |
| CD45RO CD4 | 0.0 | 0.0 | Coronery artery | 0.0 | 0.0 |
| lymphocyte act | | | SMC TNF alpha + | | |
| | | | IL-1beta | | |
| CD8 lymphocyte act | 0.0 | 0.0 | Astrocytes rest | 0.0 | 0.0 |
| Secondary CD8 | 0.0 | 0.0 | Astrocytes TNF alpha + | 0.0 | 0.0 |
| lymphocyte rest | | | IL-1beta | | |
| Secondary CD8 | 0.0 | 0.0 | KU-812 (Basophil) | 0.0 | 0.0 |
| lymphocyte act | | | rest | | |
| CD4 lymphocyte | 0.0 | 0.0 | KU-812 (Basophil) | 0.0 | 0.0 |
| none | | | PMA/ionomycin | | |
| 2ry | 0.0 | 0.0 | CCD1106 | 0.0 | 0.0 |
| Th1/Th2/Tr1_anti- | | | (Keratinocytes) none | | |
| CD95 CH11 | | | | | |
| LAK cells rest | 0.0 | 0.0 | CCD1106 | 0.0 | 0.0 |
| | | | (Keratinocytes) | | |
| | | | TNF alpha + IL-1beta | | |
| LAK cells IL-2 | 0.0 | 0.0 | Liver cirrhosis | 13.9 | 31.0 |
| LAK cells IL-2 + IL- | 0.0 | 0.0 | NCI-H292 none | 0.0 | 0.0 |
| 12 | | | | | |
| LAK cells IL-2 + IFN | 0.0 | 0.0 | NCI-H292 IL-4 | 0.0 | 0.0 |
| gamma | | | | | |
| LAK cells IL-2 + IL- | 0.0 | 0.0 | NCI-H292 IL-9 | 0.0 | 0.0 |
| 18 | | | | | |
| LAK cells | 0.0 | 0.0 | NCI-H292 IL-13 | 0.4 | 0.0 |
| PMA/ionomycin | | | | | |
| NK Cells IL-2 rest | 0.0 | 0.0 | NCI-H292 IFN | 0.0 | 0.0 |
| | | | gamma | | |
| Two Way MLR 3 | 0.0 | 0.0 | HPAEC none | 0.0 | 0.0 |
| day | | | | | |
| Two Way MLR 5 | 0.0 | 0.0 | HPAEC TNF alpha + | 0.0 | 1.2 |
| day | | | IL-1beta | | |
| Two Way MLR 7 | 0.0 | 0.0 | Lung fibroblast none | 3.5 | 0.0 |
| day | | | | | |
| PBMC rest | 0.0 | 0.0 | Lung fibroblast TNF | 0.6 | 1.9 |
| | | | alpha + IL-1beta | | |
| PBMC PWM | 0.0 | 0.0 | Lung fibroblast IL-4 | 0.0 | 1.4 |
| PBMC PHA-L | 0.0 | 0.0 | Lung fibroblast IL-9 | 1.8 | 2.1 |
| Ramos (B cell) none | 0.0 | 0.0 | Lung fibroblast IL- | 2.4 | 0.0 |
| | | | 13 | | |
| Ramos (B cell) | 0.0 | 0.0 | Lung fibroblast IFN | 0.0 | 4.1 |
| ionomycin | | | gamma | | |
| B lymphocytes | 1.9 | 0.0 | Dermal fibroblast | 3.9 | 9.7 |
| PWM | | | CCD1070 rest | | |
| B lymphocytes | 0.0 | 0.0 | Dermal fibroblast | 6.0 | 6.8 |
| CD40L and IL-4 | | | CCD1070 TNF alpha | | |
| EOL-1 dbcAMP | 0.0 | 0.0 | Dermal fibroblast | 16.0 | 10.5 |
| | | | CCD1070 IL-1beta | | |
| EOL-1 dbcAMP | 0.0 | 0.0 | Dermal fibroblast | 26.2 | 57.4 |
| PMA/ionomycin | | | IFN gamma | | |
| Dendritic cells none | 0.0 | 0.0 | Dermal fibroblast IL-4 | 30.4 | 51.8 |
| Dendritic cells LPS | 0.0 | 0.0 | Dermal Fibroblasts | 45.1 | 66.0 |
| | | | rest | | |
| Dendritic cells anti- | 0.0 | 0.0 | Neutrophils | 0.0 | 4.3 |
| CD40 | | | TNFa + LPS | | |
| Monocytes rest | 0.0 | 0.0 | Neutrophils rest | 1.2 | 5.0 |
| Monocytes LPS | 0.0 | 0.0 | Colon | 14.5 | 23.5 |
| Macrophages rest | 0.0 | 0.0 | Lung | 100.0 | 100.0 |
| Macrophages LPS | 0.0 | 0.0 | Thymus | 16.4 | 22.8 |
| HUVEC none | 0.4 | 0.0 | Kidney | 48.0 | 71.2 |
| HUVEC starved | 0.0 | 0.0 | | | |

TABLE UG
Panel 5 Islet

| | Rel. Exp. (%) | | Rel. Exp. (%) |
|---|---|---|---|
| | Ag4305, Run | | Ag4305, Run |
| Tissue Name | 248029384 | Tissue Name | 248029384 |
| 97457_Patient- | 16.4 | 94709_Donor 2 AM - A_adipose | 30.1 |
| 02go_adipose | | | |
| 97476_Patient- | 52.5 | 94710_Donor 2 AM - B_adipose | 17.8 |
| 07sk_skeletal muscle | | | |
| 97477_Patient-07ut_uterus | 67.8 | 94711_Donor 2 AM - C_adipose | 6.3 |
| 97478_Patient- | 0.5 | 94712_Donor 2 AD - A_adipose | 55.1 |
| 07pl_placenta | | | |
| 99167_Bayer Patient 1 | 13.0 | 94713_Donor 2 AD - B_adipose | 88.3 |
| 97482_Patient-08ut_uterus | 81.2 | 94714_Donor 2 AD - C_adipose | 81.8 |
| 97483_Patient- | 0.0 | 94742_Donor 3 U - A_Mesenchymal | 11.3 |
| 08pl_placenta | | Stem Cells | |
| 97486_Patient- | 15.4 | 94743_Donor 3 U - B_Mesenchymal | 12.5 |
| 09sk_skeletal muscle | | Stem Cells | |
| 97487_Patient-09ut_uterus | 100.0 | 94730_Donor 3 AM - A_adipose | 47.0 |
| 97488_Patient- | 0.0 | 94731_Donor 3 AM - B_adipose | 12.5 |
| 09pl_placenta | | | |
| 97492_Patient-10ut_uterus | 63.7 | 94732_Donor 3 AM - C_adipose | 20.6 |
| 97493_Patient- | 4.6 | 94733_Donor 3 AD - A_adipose | 76.8 |
| 10pl_placenta | | | |
| 97495_Patient- | 26.4 | 94734_Donor 3 AD - B_adipose | 27.2 |
| 11go_adipose | | | |
| 97496_Patient- | 33.2 | 94735_Donor 3 AD - C_adipose | 39.0 |
| 11sk_skeletal muscle | | | |
| 97497_Patient-11ut_uterus | 62.9 | 77138_Liver_HepG2untreated | 0.0 |
| 97498_Patient- | 0.0 | 73556_Heart_Cardiac stromal cells | 0.0 |
| 11pl_placenta | | (primary) | |
| 97500_Patient- | 26.6 | 81735_Small Intestine | 50.3 |
| 12go_adipose | | | |
| 97501_Patient- | 28.7 | 72409_Kidney_Proximal Convoluted | 0.0 |
| 12sk_skeletal muscle | | Tubule | |
| 97502_Patient-12ut_uterus | 73.7 | 82685_Small intestine_Duodenum | 41.5 |
| 97503_Patient- | 0.0 | 90650_Adrenal_Adrenocortical | 12.9 |
| 12pl_placenta | | adenoma | |
| 94721_Donor 2 U — | 1.7 | 72410_Kidney_HRCE | 0.0 |
| A_Mesenchymal Stem | | | |
| Cells | | | |
| 94722_Donor 2 U — | 0.0 | 72411_Kidney_HRE | 2.4 |
| B_Mesenchymal Stem | | | |
| Cells | | | |
| 94723_Donor 2 U — | 2.1 | 73139_Uterus_Uterine smooth | 11.5 |
| C_Mesenchymal Stem | | muscle cells | |
| Cells | | | |

AI_comprehensive panel_v1.0 Summary: Ag4305 Highest expression of the CG105973-01 gene is detected in Crohn's sample (CT=28.8). Low to moderate levels of expression of this gene are detected in samples derived from osteoarthritic (OA) bone and adjacent bone as well as OA cartilage, OA synovium and OA synovial fluid samples, and in cartilage, bone, synovium and synovial fluid samples from rheumatoid arthritis patients. Low level expression is also detected in samples derived from normal lung samples, COPD lung, emphysema, atopic asthma, asthma, allergy, Crohn's disease (normal matched control and diseased), ulcerative colitis(normal matched control and diseased), and psoriasis (normal matched control and diseased). Therefore, therapeutic modulation of this gene product may ameliorate symptoms/conditions associated with autoimmune and inflammatory disorders including psoriasis, allergy, asthma, inflammatory bowel disease, rheumatoid arthritis and osteoarthritis

CNS_neurodegeneration_v1.0 Summary: Ag4305/Ag4313 Two experiments with same primer and probe set are in excellent agreements, with highest expression of the CG105973-01 gene in a hippocampus sample from Alzheimer's patient (CTs=31). This panel confirms the expression of this gene at low levels in the brains of an independent group of individuals. However, no differential expression of this gene was detected between Alzheimer's diseased postmortem brains and those of non-demented controls in this experiment. Please see Panel 1.4 for a discussion of the potential use of this gene in treatment of central nervous system disorders.

General_screening_panel_v1.4 Summary: Ag4305/Ag4313 Two experiments with same primer and probe set are in excellent agreements, with highest expression of the CG105973-01 gene in fetal lung (Cts=27.7). Although, this gene appears to be expressed mainly in the normal tissues used in this panel, significant expression of this gene is also seen in two melanoma and three CNS cancer cell lines and colon cancer tissue. Therefore, therapeutic modulation of this gene product may be beneficial in the treatment of these cancers.

Among tissues with metabolic or endocrine function, this gene is expressed at moderate levels in pancreas, adipose, adrenal gland, thyroid, pituitary gland, skeletal muscle, heart, liver and the gastrointestinal tract. Therefore, therapeutic modulation of the activity of this gene may prove useful in the treatment of endocrine/metabolically related diseases, such as obesity and diabetes.

In addition, this gene is expressed at moderate levels in all regions of the central nervous system examined, including amygdala, hippocampus, substantia nigra, thalamus, cerebellum, cerebral cortex, and spinal cord. Therefore, this gene may play a role in central nervous system disorders such as Alzheimer's disease, Parkinson's disease, epilepsy, multiple sclerosis, schizophrenia and depression and therapeutic modulation of this gene product may be useful in the treatment of these neurological disorders.

Panel 4.1D Summary: Ag4305/Ag4313 Two experiments with same primer and probe set are in excellent agreements, with highest expression of the CG105973-01 gene in lung (CTs=31-32). In addition, moderate to low levels of expression of this gene is also seen in liver cirrhosis, dermal fibroblasts and normal tissues represented by colon, thymus, and kidney. Therefore, therapeutic modulation of this gene may be useful in the treatment of autoimmune and inflammatory diseases that affect lung,colon and kidney, such as lupus erythematosus, asthma, emphysema, Crohn's disease, ulcerative colitis, and psoriasis.

The CG105973-01 gene codes for a variant of integrin alpha-8. In the kidney, the alpha8 integrin chain is expressed in glomerular mesangial cells and it plays a role in early nephrogenesis. In mice the alpha8 integrin chain maintains the integrity of the glomerular capillary tuft during mechanical stress, eg, in hypertension (Hartner et al., 2002, Am J Pathol 160 :861-7, PMID: 11891185). Therefore, therapeutic modulation of this gene may be useful in the treatment of glomerular mesangial cell related diseases such as glomerulonephritis.

Panel 5 Islet Summary: Ag4305 Highest expression of the CG105973-01 gene is detected in uterus (CT=31). This gene is expressed at moderate to low levels in tissues with metabolic or endocrine function including adipose, uterus, small intestine and kidney. Therefore, therapeutic modulation of the activity of this gene may prove useful in the treatment of endocrine/metabolically related diseases, such as obesity and diabetes.

In addition, this gene is expressed at low levels (CT=34) in human islets. Integrins are found at the insulin-secreting beta cell surface in situ. Insulin secretagogues upregulate the beta cell-surface expression of some classes of integrins (Bosco et al., 2000, Diabetes 49(2):233-43, PMID: 10868940). Thus, therapeutic modulation of this gene product may increase beta cell insulin secretion and may be useful in the treatment of Type 2 diabetes.

V. CG106915-01 and CG106924-01: Novel Nogo Receptor Isoform-2

Expression of gene CG106924-01 was assessed using the primer-probe sets Ag4329, Ag4330 and Ag6865, described in Tables VA, VB and VC. Results of the RTQ-PCR runs are shown in Tables VD, VE and VF. Please note that probe Ag4330 is specific for the variant CG106924-01.

TABLE VA

Probe Name Ag4329

| | | | Start | SEQ ID |
|---|---|---|---|---|
| Primers | Sequences | Length | Position | No |
| Forward | 5′-cctatgaccactgagggtttt-3′ | 20 | 106 | 222 |
| Probe | TET-5′-tcatcaccgatggatatctctcctct-3′-TAMRA | 26 | 128 | 223 |
| Reverse | 5′-ggagagcagggcaagattaa-3′ | 20 | 182 | 224 |

TABLE VB

Probe Name Ag4330

| | | | Start | SEQ ID |
|---|---|---|---|---|
| Primers | Sequences | Length | Position | No |
| Forward | 5′-gcttgatgaaagcaagacaga-3′ | 21 | 3270 | 225 |
| Probe | TET-5′-ctcagatctcacaaatgacctttaaaagg-3′-TAMRA | 28 | 3305 | 226 |
| Reverse | 5′-gctgcctttcttttgtgatg-3′ | 20 | 3333 | 227 |

TABLE VC

Probe Name Ag6865

| | | | Start | SEQ ID |
|---|---|---|---|---|
| Primers | Sequences | Length | Position | No |
| Forward | 5′-gctgcacttgtgatctccat-3′ | 20 | 662 | 228 |
| Probe | TET-5′-acattaagtcttctgcTCACACGCTC-3′-TAMRA | 26 | 707 | 229 |
| Reverse | 5′-atttaggtccttggcattcct-3′ | 21 | 733 | 230 |

TABLE VD
CNS_neurodegeneration_v1.0

| | Rel. Exp. (%) | Rel. Exp. (%) | | Rel. Exp. (%) | Rel. Exp. (%) |
|---|---|---|---|---|---|
| | Ag4329, Run | Ag4330, Run | | Ag4329, Run | Ag4330, Run |
| Tissue Name | 224344077 | 224344731 | Tissue Name | 224344077 | 224344731 |
| AD 1 Hippo | 21.9 | 16.5 | Control (Path) | 3.9 | 0.0 |
| | | | 3 Temporal | | |
| | | | Ctx | | |
| AD 2 Hippo | 36.9 | 8.2 | Control (Path) | 40.3 | 35.6 |
| | | | 4 Temporal | | |
| | | | Ctx | | |
| AD 3 Hippo | 13.6 | 16.7 | AD 1 | 28.1 | 15.8 |
| | | | Occipital Ctx | | |
| AD 4 Hippo | 11.7 | 2.1 | AD 2 | 0.0 | 0.0 |
| | | | Occipital Ctx | | |
| | | | (Missing) | | |
| AD 5 hippo | 96.6 | 40.9 | AD 3 | 6.2 | 4.0 |
| | | | Occipital Ctx | | |
| AD 6 Hippo | 100.0 | 17.6 | AD 4 | 25.0 | 19.1 |
| | | | Occipital Ctx | | |
| Control 2 Hippo | 11.4 | 29.9 | AD 5 | 60.7 | 43.2 |
| | | | Occipital Ctx | | |
| Control 4 Hippo | 18.0 | 13.5 | AD 6 | 39.2 | 19.2 |
| | | | Occipital Ctx | | |
| Control (Path) 3 | 6.0 | 5.8 | Control 1 | 0.0 | 1.1 |
| Hippo | | | Occipital Ctx | | |
| AD 1 Temporal | 7.2 | 5.4 | Control 2 | 72.2 | 51.4 |
| Ctx | | | Occipital Ctx | | |
| AD 2 Temporal | 18.8 | 47.3 | Control 3 | 33.4 | 16.0 |
| Ctx | | | Occipital Ctx | | |
| AD 3 Temporal | 0.0 | 6.0 | Control 4 | 9.7 | 1.3 |
| Ctx | | | Occipital Ctx | | |
| AD 4 Temporal | 19.8 | 16.6 | Control (Path) | 85.9 | 81.8 |
| Ctx | | | 1 Occipital | | |
| | | | Ctx | | |
| AD 5 Inf | 53.6 | 100.0 | Control (Path) | 17.3 | 19.3 |
| Temporal Ctx | | | 2 Occipital | | |
| | | | Ctx | | |
| AD 5 | 23.8 | 33.7 | Control (Path) | 0.0 | 1.9 |
| Sup Temporal Ctx | | | 3 Occipital | | |
| | | | Ctx | | |
| AD 6 Inf | 72.2 | 27.2 | Control (Path) | 23.0 | 23.5 |
| Temporal Ctx | | | 4 Occipital | | |
| | | | Ctx | | |
| AD 6 Sup | 54.7 | 25.9 | Control 1 | 4.9 | 10.6 |
| Temporal Ctx | | | Parietal Ctx | | |
| Control 1 | 6.8 | 7.0 | Control 2 | 46.0 | 26.1 |
| Temporal Ctx | | | Parietal Ctx | | |
| Control 2 | 49.0 | 44.4 | Control 3 | 39.2 | 11.0 |
| Temporal Ctx | | | Parietal Ctx | | |
| Control 3 | 28.7 | 3.3 | Control (Path) | 95.9 | 54.3 |
| Temporal Ctx | | | 1 Parietal Ctx | | |
| Control 4 | 9.5 | 1.6 | Control (Path) | 53.2 | 42.0 |
| Temporal Ctx | | | 2 Parietal Ctx | | |
| Control (Path) 1 | 40.9 | 50.3 | Control (Path) | 8.1 | 1.9 |
| Temporal Ctx | | | 3 Parietal Ctx | | |
| Control (Path) 2 | 48.0 | 23.8 | Control (Path) | 64.6 | 37.9 |
| Temporal Ctx | | | 4 Parietal Ctx | | |

TABLE VE
General_screening_panel_v1.4

| | Rel. Exp. (%) | Rel. Exp. (%) | | Rel. Exp. (%) | Rel. Exp. (%) |
|---|---|---|---|---|---|
| | Ag4329, Run | Ag4330, Run | | Ag4329, Run | Ag4330, Run |
| Tissue Name | 222550606 | 222550615 | Tissue Name | 222550606 | 222550615 |
| Adipose | 0.0 | 0.0 | Renal ca. TK-10 | 0.0 | 0.0 |
| Melanoma* | 0.0 | 0.0 | Bladder | 0.7 | 2.7 |
| Hs688(A).T | | | | | |
| Melanoma* | 0.0 | 0.0 | Gastric ca. (liver | 1.9 | 0.0 |
| Hs688(B).T | | | met.) NCI-N87 | | |
| Melanoma* | 0.0 | 0.0 | Gastric ca. KATO | 0.0 | 0.0 |
| M14 | | | III | | |
| Melanoma* | 0.0 | 0.0 | Colon ca. SW-948 | 0.0 | 0.0 |
| LOXIMVI | | | | | |
| Melanoma* SK- | 0.0 | 0.0 | Colon ca. SW480 | 0.0 | 0.0 |
| MEL-5 | | | | | |
| Squamous cell | 0.0 | 0.0 | Colon ca.* (SW480 | 0.0 | 0.0 |
| carcinoma SCC-4 | | | met) SW620 | | |
| Testis Pool | 3.2 | 5.1 | Colon ca. HT29 | 0.0 | 3.0 |
| Prostate ca.* | 0.0 | 0.0 | Colon ca. HCT-116 | 0.0 | 0.0 |
| (bone met) PC-3 | | | | | |
| Prostate Pool | 0.4 | 1.7 | Colon ca. CaCo-2 | 0.0 | 0.0 |
| Placenta | 0.0 | 0.0 | Colon cancer tissue | 0.0 | 0.0 |
| Uterus Pool | 0.0 | 1.0 | Colon ca. SW1116 | 0.0 | 0.0 |
| Ovarian ca. | 0.0 | 0.6 | Colon ca. Colo-205 | 0.0 | 0.0 |
| OVCAR-3 | | | | | |
| Ovarian ca. SK- | 0.0 | 1.0 | Colon ca. SW-48 | 0.0 | 0.0 |
| OV-3 | | | | | |
| Ovarian ca. | 0.0 | 0.0 | Colon Pool | 0.8 | 1.4 |
| OVCAR-4 | | | | | |
| Ovarian ca. | 0.0 | 0.0 | Small Intestine | 1.2 | 2.6 |
| OVCAR-5 | | | Pool | | |
| Ovarian ca. | 0.0 | 0.9 | Stomach Pool | 0.4 | 2.5 |
| IGROV-1 | | | | | |
| Ovarian ca. | 0.0 | 0.0 | Bone Marrow Pool | 0.3 | 0.8 |
| OVCAR-8 | | | | | |
| Ovary | 0.9 | 0 6 | Fetal Heart | 100.0 | 100.0 |
| Breast ca. MCF-7 | 0.0 | 0.0 | Heart Pool | 10.2 | 16.5 |
| Breast ca. | 0.0 | 0.0 | Lymph Node Pool | 5.0 | 2.3 |
| MDA-MB-231 | | | | | |
| Breast ca. BT | 0.4 | 0.0 | Fetal Skeletal | 0.0 | 0.0 |
| 549 | | | Muscle | | |
| Breast ca. T47D | 0.0 | 1.6 | Skeletal Muscle | 1.7 | 1.1 |
| | | | Pool | | |
| Breast ca. | 0.0 | 0.0 | Spleen Pool | 1.2 | 1.0 |
| MDA-N | | | | | |
| Breast Pool | 1.8 | 3.1 | Thymus Pool | 1.2 | 4.3 |
| Trachea | 0 0 | 0.0 | CNS cancer | 0.0 | 0.0 |
| | | | (glio/astro) U87- | | |
| | | | MG | | |
| Lung | 1.0 | 1.6 | CNS cancer | 0.0 | 0.0 |
| | | | (glio/astro) U-118- | | |
| | | | MG | | |
| Fetal Lung | 0.4 | 0.4 | CNS cancer | 0.0 | 1.2 |
| | | | (neuro;met) SK-N- | | |
| | | | AS | | |
| Lung ca. NCI- | 0.0 | 0.0 | CNS cancer (astro) | 0.0 | 0.0 |
| N417 | | | SF-539 | | |
| Lung ca. LX-1 | 0.0 | 0.0 | CNS cancer (astro) | 0.5 | 0.0 |
| | | | SNB-75 | | |
| Lung ca. NCI- | 2.4 | 5.4 | CNS cancer (glio) | 0.0 | 0.0 |
| H146 | | | SNB-19 | | |
| Lung ca. SHP- | 17.0 | 47.3 | CNS cancer (glio) | 0.0 | 0.0 |
| 77 | | | SF-295 | | |
| Lung ca. A549 | 0.0 | 0.0 | Brain (Amygdala) | 2.8 | 2.9 |
| | | | Pool | | |
| Lung ca. NCI- | 0.0 | 0.0 | Brain (cerebellum) | 0.9 | 1.5 |
| H526 | | | | | |
| Lung ca. NCI- | 0.0 | 0.0 | Brain (fetal) | 2.3 | 3.1 |
| H23 | | | | | |
| Lung ca. NCI- | 0.5 | 0.7 | Brain | 3.7 | 6.7 |
| H460 | | | (Hippocampus) | | |
| | | | Pool | | |
| Lung ca. HOP- | 0.3 | 3.1 | Cerebral Cortex | 7.7 | 16.2 |
| 62 | | | Pool | | |
| Lung ca. NCI- | 0.2 | 0.0 | Brain (Substantia | 4.6 | 8.1 |
| H522 | | | nigra) Pool | | |
| Liver | 0.0 | 0.0 | Brain (Thalamus) | 8.8 | 17.9 |
| | | | Pool | | |
| Fetal Liver | 0.0 | 0.0 | Brain (whole) | 3.0 | 2.6 |
| Liver ca. | 0.0 | 0.0 | Spinal Cord Pool | 2.2 | 2.7 |
| HepG2 | | | | | |
| Kidney Pool | 6.3 | 6.5 | Adrenal Gland | 0.0 | 0.0 |
| Fetal Kidney | 2.1 | 0.5 | Pituitary gland | 0.0 | 0.4 |
| | | | Pool | | |
| Renal ca. 786-0 | 0.0 | 0.0 | Salivary Gland | 0.0 | 0.0 |
| Renal ca. A498 | 0.3 | 0.7 | Thyroid (female) | 0.0 | 0.6 |
| Renal ca. | 0.0 | 0.0 | Pancreatic ca. | 0.0 | 0.0 |
| ACHN | | | CAPAN2 | | |
| Renal ca. UO- | 0.0 | 0.0 | Pancreas Pool | 1.4 | 4.7 |
| 31 | | | | | |

TABLE VF
General_screening_panel_v1.6

| | Rel. Exp. (%) | | Rel. Exp. (%) |
|---|---|---|---|
| | Ag6865, Run | | Ag6865, Run |
| Tissue Name | 278387549 | Tissue Name | 278387549 |
| Adipose | 4.4 | Renal ca. TK-10 | 0.0 |
| Melanoma* | 0.0 | Bladder | 1.0 |
| Hs688(A).T | | | |
| Melanoma* | 0.0 | Gastric ca. (liver met.) | 1.2 |
| Hs688(B).T | | NCI-N87 | |
| Melanoma* M14 | 0.0 | Gastric ca. KATO III | 0.0 |
| Melanoma* LOXIMVI | 0.0 | Colon ca. SW-948 | 0.0 |
| Melanoma* SK-MEL-5 | 0.0 | Colon ca. SW480 | 0.0 |
| Squamous cell | 0.0 | Colon ca.* (SW480 met) | 0.0 |
| carcinoma SCC-4 | | SW620 | |
| Testis Pool | 6.3 | Colon ca. HT29 | 0.5 |
| Prostate ca.* (bone met) | 0.4 | Colon ca. HCT-116 | 0.0 |
| PC-3 | | | |
| Prostate Pool | 0.0 | Colon ca. CaCo-2 | 0.0 |
| Placenta | 0.0 | Colon cancer tissue | 0.0 |
| Uterus Pool | 1.3 | Colon ca. SW1116 | 0.0 |
| Ovarian ca. OVCAR-3 | 0.0 | Colon ca. Colo-205 | 0.0 |
| Ovarian ca. SK-OV-3 | 0.0 | Colon ca. SW-48 | 0.0 |
| Ovarian ca. OVCAR-4 | 0.0 | Colon Pool | 3.1 |
| Ovarian ca. OVCAR-5 | 0.0 | Small Intestine Pool | 2.4 |
| Ovarian ca. IGROV-1 | 0.0 | Stomach Pool | 0.0 |
| Ovarian ca. OVCAR-8 | 0.0 | Bone Marrow Pool | 0.6 |
| Ovary | 0.9 | Fetal Heart | 100.0 |
| Breast ca. MCF-7 | 0.0 | Heart Pool | 12.1 |
| Breast ca. MDA-MB- | 0.0 | Lymph Node Pool | 1.7 |
| 231 | | | |
| Breast ca. BT 549 | 0.0 | Fetal Skeletal Muscle | 0.7 |
| Breast ca. T47D | 0.0 | Skeletal Muscle Pool | 1.8 |
| Breast ca. MDA-N | 0.0 | Spleen Pool | 1.5 |
| Breast Pool | 0.0 | Thymus Pool | 1.6 |
| Trachea | 0.7 | CNS cancer (glio/astro) | 0.0 |
| | | U87-MG | |
| Lung | 0.7 | CNS cancer (glio/astro) U- | 0.0 |
| | | 118-MG | |
| Fetal Lung | 0.6 | CNS cancer (neuro; met) | 0.7 |
| | | SK-N-AS | |
| Lung ca. NCI-N417 | 0.0 | CNS cancer (astro) SF-539 | 0.0 |
| Lung ca. LX-1 | 0.0 | CNS cancer (astro) SNB- | 0.0 |
| | | 75 | |
| Lung ca. NCI-H146 | 2.5 | CNS cancer (glio) SNB-19 | 0.0 |
| Lung ca. SHP-77 | 16.5 | CNS cancer (glio) SF-295 | 0.0 |
| Lung ca. A549 | 0.0 | Brain (Amygdala) Pool | 3.6 |
| Lung ca. NCI-H526 | 0.0 | Brain (cerebellum) | 4.2 |
| Lung ca. NCI-H23 | 0.0 | Brain (fetal) | 3.6 |
| Lung ca. NCI-H460 | 0.0 | Brain (Hippocampus) Pool | 2.7 |
| Lung ca. HOP-62 | 1.5 | Cerebral Cortex Pool | 8.4 |
| Lung ca. NCI-H522 | 0.0 | Brain (Substantia nigra) | 3.3 |
| | | Pool | |
| Liver | 0.0 | Brain (Thalamus) Pool | 7.9 |
| Fetal Liver | 0.6 | Brain (whole) | 1.1 |
| Liver ca. HepG2 | 0.0 | Spinal Cord Pool | 2.4 |
| Kidney Pool | 5.9 | Adrenal Gland | 0.0 |
| Fetal Kidney | 1.8 | Pituitary gland Pool | 1.2 |
| Renal ca. 786-0 | 0.0 | Salivary Gland | 0.0 |
| Renal ca. A498 | 1.7 | Thyroid (female) | 0.0 |
| Renal ca. ACHN | 0.0 | Pancreatic ca. CAPAN2 | 0.0 |
| Renal ca. UO-31 | 0.0 | Pancreas Pool | 0.0 |

CNS_neurodegeneration_v1.0 Summary: Ag4329/Ag4330 Two experiments with two different probe and primer sets are in good agreement with significant expression of the CG106924-01 gene in the brains of an independent group of individuals. However, no differential expression of this gene was detected between Alzheimer's diseased postmortem brains and those of non-demented controls in this experiment. Please see Panel 1.4 for a discussion of the potential use of this gene in treatment of central nervous system disorders.

General_screening_panel_v1.4 Summary: Ag4329/Ag4330 Two experiment with different probe and primer sets are in excellent agreements with highest expression of the CG106924-01 gene in fetal heart (CT=30.5-32). Interestingly, expression of this gene is higher in fetal as compared to the adult heart (CT=33.8-34.6). Therefore, expression of this gene may be used to distinguish fetal heart from adult tissue and also from other samples used in this panel. In addition, the relative overexpression of this gene in fetal heart suggests that the protein product may enhance heart growth or development in the fetus and thus may also act in a regenerative capacity in the adult. Therefore, therapeutic modulation of the protein encoded by this gene could be useful in treatment of heart related diseases.

In addition, this gene is expressed at low levels in some of the regions of the central nervous system examined, including substantia nigra, thalamus, and cerebral cortex. This gene encodes a leucine-rich repeat protein. Leucine rich repeats (LRR) mediate reversible protein-protein interactions and have diverse cellular functions, including cellular adhesion and signaling. Several of these proteins, such as connectin, slit, chaoptin, and Toll have been shown to have a pivotal role in neuronal development in Drosophila, as well as a distinct role in neural development and in the adult nervous system of humans (Battye R., 2001, J. Neurosci. 21: 4290-4298; Itoh A., 1998, Brain Res. Mol. Brain Res. 62: 175-186). In Drosophilia, the LRR region of axon guidance proteins has been shown to be critical for their function (especially in axon repulsion). Since the leucine-rich-repeat protein encoded by this gene shows significant expression in the cerebral cortex, it is an excellent candidate neuronal guidance protein for axons, dendrites and/or growth cones in general. Therefore, therapeutic modulation of the levels of this protein, or possible signaling via this protein, may be of utility in enhancing/directing compensatory synaptogenesis and fiber growth in the CNS in response to neuronal death (stroke, head trauma), axon lesion (spinal cord injury), or neurodegeneration (Alzheimer's, Parkinson's, Huntington's, vascular dementia or any neurodegenerative disease).

General_screening_panel_v1.6 Summary: Ag6865 Highest expression of the CG106915-01 gene is detected in fetal heart (CT=30.2). Interestingly, expression of this gene is higher in fetal as compared to the adult heart (CT=33.3). Therefore, expression of this gene may be used to distinguish fetal heart from adult tissue and also from other samples used in this panel. In addition, the relative overexpression of this gene in fetal heart suggests that the protein product may enhance heart growth or development in the fetus and thus may also act in a regenerative capacity in the adult. Therefore, therapeutic modulation of the protein encoded by this gene could be useful in treatment of heart related diseases.

In addition, this gene is expressed at low levels in some of the regions of the central nervous system examined, including thalamus, and cerebral cortex. Please see Panel 1.4 for a discussion of the potential use of this gene in treatment of central nervous system disorders.

Low expression of this gene is also seen in a lung cancer SHP-77 cell line. Therefore, expression of this gene may be used as a marker to detect the presence of lung cancer and therapeutic modulation of this gene may be useful in the treatment of this cancer.

Low expression of this gene is also seen in kidney, testis and adipose tissue. Therefore, therapeutic modulation of this gene may be useful in the treatment of diseases associated with these tissues including obesity, diabetes, lupus, glomerulonephritis, fertility and hypogonadism.

W. CG106942-01: Nramp-like Membrane Protein

Expression of gene CG106942-01 was assessed using the primer-probe set Ag4331, described in Table WA. Results of the RTQ-PCR runs are shown in Tables WB, WC and WD.

TABLE WA

Probe Name Ag4331

| | | | Start | SEQ ID |
|---|---|---|---|---|
| Primers | Sequences | Length | Position | No |
| Forward | 5′-gctgtttgtttggagtcgtcta-3′ | 22 | 1314 | 231 |
| Probe | TET-5′-cttcttcggttaccgctgcttcaagg-3′-TAMRA | 26 | 1339 | 232 |
| Reverse | 5′-aacagcaacccagtgagaaag-3′ | 21 | 1372 | 233 |

TABLE WB
CNS_neurodegeneration_v1.0

| | Rel. Exp. (%) | | Rel. Exp. (%) |
|---|---|---|---|
| | Ag4331, Run | | Ag4331, Run |
| Tissue Name | 224344734 | Tissue Name | 224344734 |
| AD 1 Hippo | 8.8 | Control (Path) 3 | 8.2 |
| | | Temporal Ctx | |
| AD 2 Hippo | 19.2 | Control (Path) 4 | 18.8 |
| | | Temporal Ctx | |
| AD 3 Hippo | 13.2 | AD 1 Occipital Ctx | 1.2 |
| AD 4 Hippo | 10.9 | AD 2 Occipital Ctx | 0.0 |
| | | (Missing) | |
| AD 5 hippo | 56.6 | AD 3 Occipital Ctx | 5.1 |
| AD 6 Hippo | 40.9 | AD 4 Occipital Ctx | 13.3 |
| Control 2 Hippo | 29.3 | AD 5 Occipital Ctx | 8.5 |
| Control 4 Hippo | 13.4 | AD 6 Occipital Ctx | 36.3 |
| Control (Path) 3 Hippo | 1.8 | Control 1 Occipital | 2.8 |
| | | Ctx | |
| AD 1 Temporal Ctx | 7.1 | Control 2 Occipital | 59.5 |
| | | Ctx | |
| AD 2 Temporal Ctx | 20.2 | Control 3 Occipital | 2.2 |
| | | Ctx | |
| AD 3 Temporal Ctx | 5.8 | Control 4 Occipital | 8.9 |
| | | Ctx | |
| AD 4 Temporal Ctx | 9.5 | Control (Path) 1 | 62.0 |
| | | Occipital Ctx | |
| AD 5 Inf Temporal Ctx | 68.8 | Control (Path) 2 | 5.6 |
| | | Occipital Ctx | |
| AD 5 SupTemporal Ctx | 28.3 | Control (Path) 3 | 2.1 |
| | | Occipital Ctx | |
| AD 6 Inf Temporal Ctx | 39.5 | Control (Path) 4 | 13.8 |
| | | Occipital Ctx | |
| AD 6 Sup Temporal Ctx | 21.2 | Control 1 Parietal Ctx | 4.6 |
| Control 1 Temporal Ctx | 2.2 | Control 2 Parietal Ctx | 15.9 |
| Control 2 Temporal Ctx | 91.4 | Control 3 Parietal Ctx | 17.8 |
| Control 3 Temporal Ctx | 8.2 | Control (Path) 1 | 100.0 |
| | | Parietal Ctx | |
| Control 4 Temporal Ctx | 8.4 | Control (Path) 2 | 14.1 |
| | | Parietal Ctx | |
| Control (Path) 1 | 37.1 | Control (Path) 3 | 2.3 |
| Temporal Ctx | | Parietal Ctx | |
| Control (Path) 2 | 4.1 | Control (Path) 4 | 21.3 |
| Temporal Ctx | | Parietal Ctx | |

TABLE WC
General_screening_panel_v1.4

| | Rel. Exp. (%) | | Rel. Exp. (%) |
|---|---|---|---|
| | Ag4331, Run | | Ag4331, Run |
| Tissue Name | 222556053 | Tissue Name | 222556053 |
| Adipose | 0.2 | Renal ca. TK-10 | 31.0 |
| Melanoma* | 2.4 | Bladder | 3.8 |
| Hs688(A).T | | | |
| Melanoma* | 1.2 | Gastric ca. (liver met.) | 4.2 |
| Hs688(B).T | | NCI-N87 | |
| Melanoma* M14 | 24.7 | Gastric ca. KATO III | 1.7 |
| Melanoma* LOXIMVI | 7.8 | Colon ca. SW-948 | 0.7 |
| Melanoma* SK-MEL-5 | 3.5 | Colon ca. SW480 | 83.5 |
| Squamous cell | 3.1 | Colon ca.* (SW480 met) | 10.7 |
| carcinoma SCC-4 | | SW620 | |
| Testis Pool | 3.8 | Colon ca. HT29 | 1.2 |
| Prostate ca.* (bone met) | 2.1 | Colon ca. HCT-116 | 4.4 |
| PC-3 | | | |
| Prostate Pool | 2.0 | Colon ca. CaCo-2 | 29.7 |
| Placenta | 4.0 | Colon cancer tissue | 24.0 |
| Uterus Pool | 0.2 | Colon ca. SW1116 | 4.2 |
| Ovarian ca. OVCAR-3 | 1.9 | Colon ca. Colo-205 | 2.2 |
| Ovarian ca. SK-OV-3 | 2.0 | Colon ca. SW-48 | 17.9 |
| Ovarian ca. OVCAR-4 | 7.6 | Colon Pool | 1.3 |
| Ovarian ca. OVCAR-5 | 16.8 | Small Intestine Pool | 0.9 |
| Ovarian ca. IGROV-1 | 12.8 | Stomach Pool | 2.0 |
| Ovarian ca. OVCAR-8 | 7.1 | Bone Marrow Pool | 0.1 |
| Ovary | 4.7 | Fetal Heart | 1.8 |
| Breast ca. MCF-7 | 11.7 | Heart Pool | 0.5 |
| Breast ca. MDA-MB- | 4.3 | Lymph Node Pool | 2.2 |
| 231 | | | |
| Breast ca. BT 549 | 10.4 | Fetal Skeletal Muscle | 0.8 |
| Breast ca. T47D | 51.8 | Skeletal Muscle Pool | 0.1 |
| Breast ca. MDA-N | 31.4 | Spleen Pool | 2.5 |
| Breast Pool | 1.7 | Thymus Pool | 1.6 |
| Trachea | 1.2 | CNS cancer (glio/astro) | 8.5 |
| | | U87-MG | |
| Lung | 1.0 | CNS cancer (glio/astro) U- | 6.8 |
| | | 118-MG | |
| Fetal Lung | 1.6 | CNS cancer (neuro; met) | 21.6 |
| | | SK-N-AS | |
| Lung ca. NCI-N417 | 60.3 | CNS cancer (astro) SF-539 | 2.5 |
| Lung ca. LX-1 | 13.6 | CNS cancer (astro) SNB- | 32.8 |
| | | 75 | |
| Lung ca. NCI-H146 | 42.9 | CNS cancer (glio) SNB-19 | 8.7 |
| Lung ca. SHP-77 | 100.0 | CNS cancer (glio) SF-295 | 2.0 |
| Lung ca. A549 | 0.8 | Brain (Amygdala) Pool | 10.7 |
| Lung ca. NCI-H526 | 58.2 | Brain (cerebellum) | 39.5 |
| Lung ca. NCI-H23 | 4.0 | Brain (fetal) | 37.4 |
| Lung ca. NCI-H460 | 9.5 | Brain (Hippocampus) Pool | 32.8 |
| Lung ca. HOP-62 | 0.6 | Cerebral Cortex Pool | 26.6 |
| Lung ca. NCI-H522 | 16.0 | Brain (Substantia nigra) | 34.9 |
| | | Pool | |
| Liver | 0.3 | Brain (Thalamus) Pool | 21.2 |
| Fetal Liver | 2.6 | Brain (whole) | 34.9 |
| Liver ca. HepG2 | 64.6 | Spinal Cord Pool | 6.8 |
| Kidney Pool | 2.5 | Adrenal Gland | 8.2 |
| Fetal Kidney | 1.7 | Pituitary gland Pool | 3.6 |
| Renal ca. 786-0 | 8.0 | Salivary Gland | 0.8 |
| Renal ca. A498 | 18.4 | Thyroid (female) | 4.7 |
| Renal ca. ACHN | 11.3 | Pancreatic ca. CAPAN2 | 1.6 |
| Renal ca. UO-31 | 2.5 | Pancreas Pool | 4.3 |

TABLE WD
Panel 4.1D

| | Rel. Exp. (%) | | Rel. Exp. (%) |
|---|---|---|---|
| | Ag4331, Run | | Ag4331, Run |
| Tissue Name | 183718671 | Tissue Name | 183718671 |
| Secondary Th1 act | 19.2 | HUVEC IL-1beta | 0.0 |
| Secondary Th2 act | 63.7 | HUVEC IFN gamma | 0.0 |
| Secondary Tr1 act | 13.2 | HUVEC TNF alpha + IFN | 0.0 |
| | | gamma | |
| Secondary Th1 rest | 4.0 | HUVEC TNF alpha + IL4 | 0.0 |
| Secondary Th2 rest | 0.0 | HUVEC IL-11 | 0.0 |
| Secondary Tr1 rest | 1.5 | Lung Microvascular EC none | 0.0 |
| Primary Th1 act | 24.0 | Lung Microvascular EC | 0.0 |
| | | TNF alpha + IL-1beta | |
| Primary Th2 act | 47.3 | Microvascular Dermal EC | 0.0 |
| | | none | |
| Primary Tr1 act | 7.0 | Microsvasular Dermal EC | 0.0 |
| | | TNF alpha + IL-1beta | |
| Primary Th1 rest | 0.0 | Bronchial epithelium | 10.1 |
| | | TNF alpha + IL1beta | |
| Primary Th2 rest | 4.7 | Small airway epithelium none | 0.0 |
| Primary Tr1 rest | 11.8 | Small airway epithelium | 7.9 |
| | | TNF alpha + IL-1beta | |
| CD45RA CD4 | 55.5 | Coronery artery SMC rest | 8.9 |
| lymphocyte act | | | |
| CD45RO CD4 | 25.3 | Coronery artery SMC | 8.0 |
| lymphocyte act | | TNF alpha + IL-1beta | |
| CD8 lymphocyte act | 21.3 | Astrocytes rest | 39.0 |
| Secondary CD8 | 18.6 | Astrocytes TNF alpha + IL- | 25.3 |
| lymphocyte rest | | 1beta | |
| Secondary CD8 | 12.9 | KU-812 (Basophil) rest | 0.0 |
| lymphocyte act | | | |
| CD4 lymphocyte none | 0.0 | KU-812 (Basophil) | 1.4 |
| | | PMA/ionomycin | |
| 2ry Th1/Th2/Tr1_anti- | 14.2 | CCD1106 (Keratinocytes) | 11.6 |
| CD95 CH11 | | none | |
| LAK cells rest | 12.0 | CCD1106 (Keratinocytes) | 8.7 |
| | | TNF alpha + IL-1beta | |
| LAK cells IL-2 | 25.3 | Liver cirrhosis | 6.9 |
| LAK cells IL-2 + IL-12 | 15.3 | NCI-H292 none | 67.8 |
| LAK cells IL-2 + IFN | 27.2 | NCI-H292 IL-4 | 100.0 |
| gamma | | | |
| LAK cells IL-2 + IL-18 | 6.6 | NCI-H292 IL-9 | 75.3 |
| LAK cells | 0.0 | NCI-H292 IL-13 | 50.7 |
| PMA/ionomycin | | | |
| NK Cells IL-2 rest | 22.4 | NCI-H292 IFN gamma | 77.9 |
| Two Way MLR 3 day | 8.9 | HPAEC none | 0.0 |
| Two Way MLR 5 day | 23.5 | HPAEC TNF alpha + IL-1beta | 0.0 |
| Two Way MLR 7 day | 10.5 | Lung fibroblast none | 9.9 |
| PBMC rest | 0.0 | Lung fibroblast TNF alpha + | 50.0 |
| | | IL-1beta | |
| PBMC PWM | 52.5 | Lung fibroblast IL-4 | 7.7 |
| PBMC PHA-L | 33.7 | Lung fibroblast IL-9 | 13.3 |
| Ramos (B cell) none | 5.9 | Lung fibroblast IL-13 | 17.4 |
| Ramos (B cell) ionomycin | 10.4 | Lung fibroblast IFN gamma | 26.6 |
| B lymphocytes PWM | 28.3 | Dermal fibroblast CCD1070 | 28.9 |
| | | rest | |
| B lymphocytes CD40L | 18.0 | Dermal fibroblast CCD1070 | 19.1 |
| and IL-4 | | TNF alpha | |
| EOL-1 dbcAMP | 0.0 | Dermal fibroblast CCD1070 | 16.6 |
| | | IL-1beta | |
| EOL-1 dbcAMP | 2.3 | Dermal fibroblast IFN gamma | 13.0 |
| PMA/ionomycin | | | |
| Dendritic cells none | 0.0 | Dermal fibroblast IL-4 | 26.6 |
| Dendritic cells LPS | 0.0 | Dermal Fibroblasts rest | 22.1 |
| Dendritic cells anti-CD40 | 0.0 | Neutrophils TNF a + LPS | 0.0 |
| Monocytes rest | 0.0 | Neutrophils rest | 0.0 |
| Monocytes LPS | 0.0 | Colon | 1.6 |
| Macrophages rest | 0.0 | Lung | 0.0 |
| Macrophages LPS | 0.0 | Thymus | 14.7 |
| HUVEC none | 0.0 | Kidney | 24.5 |
| HUVEC starved | 0.0 | | |

CNS_neurodegeneration_v1.0 Summary: Ag4331 This panel confirms the expression of the CG106942-01 gene at low levels in the brains of an independent group of individuals. However, no differential expression of this gene was detected between Alzheimer's diseased postmortem brains and those of non-demented controls in this experiment. Please see Panel 1.4 for a discussion of the potential use of this gene in treatment of central nervous system disorders.

General_screening_panel_v1.4 Summary: Ag4331 Highest expression of the CG106942-01 gene is detected in a lung cancer SHP-77 cell line (CT=28.5). High to moderate levels of expression of this gene is also seen in cluster of cancer cell lines including CNS, colon, renal, liver, breast, ovarian and melanoma cancer cell lines. Therefore, expression of this gene may be used as diagnostic marker for detection of these cancers and therapeutic modulation of this gene product may be beneficial in the treatment of these cancers.

Among tissues with metabolic or endocrine function, this gene is expressed at moderate to low levels in pancreas, adrenal gland, thyroid, pituitary gland, fetal heart, fetal liver and the gastrointestinal tract. Therefore, therapeutic modulation of the activity of this gene may prove useful in the treatment of endocrine/metabolically related diseases, such as obesity and diabetes.

Interestingly, expression of this gene is higher in fetal (CT=33.8) as compared to adult liver (CT=37). Therefore, expression of this gene may be used to distinguish the fetal tissue from the adult liver. In addition, the relative overexpression of this gene in fetal liver suggests that the protein product may enhance liver growth or development in the fetus and thus may also act in a regenerative capacity in the adult. Therefore, therapeutic modulation of the protein encoded by this gene could be useful in treatment of liver related diseases.

In addition, this gene is expressed at high to moderate levels in all regions of the central nervous system examined, including amygdala, hippocampus, substantia nigra, thalamus, cerebellum, cerebral cortex, and spinal cord. Therefore, this gene may play a role in central nervous system disorders such as Alzheimer's disease, Parkinson's disease, epilepsy, multiple sclerosis, schizophrenia and depression.

Panel 4.1D Summary: Ag4331 Highest expression of the CG106942-01 gene is detected in IL4 treated NCI-H292 cells (CT=33.4). In addition, moderate to low expression of this gene is also seen in activated primary and secondary Th2 cells, activated CD45RA CD4 lymphocytes, PWM/PHA-L treated PBMC cells, resting astrocytes, untreated and cytokine treated NCI-H292 cells, TNF alpha+IL-1 beta treated lung fibroblasts. Therefore, therapeutic modulation of this gene product may be beneficial in the treatment of T cells and B cells mediated diseases such as systemic lupus erythematosus, Crohn's disease, ulcerative colitis, multiple sclerosis, chronic obstructive pulmonary disease, asthma, emphysema, rheumatoid arthritis, or psoriasis.

X. CG107513-01: Syntaxin Domain Containing Protein

Expression of gene CG107513-01 was assessed using the primer-probe set Ag4339, described in Table XA. Results of the RTQ-PCR runs are shown in Tables XB, XC and XD.

TABLE XA

Probe Name Ag4339

| | | | | |
|---|---|---|---|---|
| | | | Start | SEQ ID |
| Primers | Sequences | Length | Position | No |

| | | | | |
|---|---|---|---|---|
| Forward | 5′-gacgacaatgtggcagagtatc-3′ | 22 | 957 | 234 |
| Probe | TET-5′-aaactggccaacaacgcggacaag-3′-TAMRA | 24 | 981 | 235 |
| Reverse | 5′-tcttctcgaacacttgcttgat-3′ | 22 | 1020 | 236 |

TABLE XB
CNS_neurodegeneration_v1.0

| | Rel. Exp. (%) | | Rel. Exp. (%) |
|---|---|---|---|
| | Ag4339, Run | | Ag4339, Run |
| Tissue Name | 224358973 | Tissue Name | 224358973 |
| AD 1 Hippo | 7.7 | Control (Path) 3 | 2.7 |
| | | Temporal Ctx | |
| AD 2 Hippo | 16.5 | Control (Path) 4 | 29.5 |
| | | Temporal Ctx | |
| AD 3 Hippo | 2.2 | AD 1 Occipital Ctx | 4.8 |
| AD 4 Hippo | 2.4 | AD 2 Occipital Ctx | 0.0 |
| | | (Missing) | |
| AD 5 Hippo | 97.3 | AD 3 Occipital Ctx | 2.0 |
| AD 6 Hippo | 33.2 | AD 4 Occipital Ctx | 18.2 |
| Control 2 Hippo | 20.7 | AD 5 Occipital Ctx | 40.9 |
| Control 4 Hippo | 2.5 | AD 6 Occipital Ctx | 19.6 |
| Control (Path) 3 Hippo | 2.4 | Control 1 Occipital Ctx | 0.8 |
| AD 1 Temporal Ctx | 8.5 | Control 2 Occipital Ctx | 60.3 |
| AD 2 Temporal Ctx | 23.8 | Control 3 Occipital Ctx | 17.4 |
| AD 3 Temporal Ctx | 2.4 | Control 4 Occipital Ctx | 2.2 |
| AD 4 Temporal Ctx | 17.0 | Control (Path) 1 | 100.0 |
| | | Occipital Ctx | |
| AD 5 Inf Temporal Ctx | 86.5 | Control (Path) 2 | 15.7 |
| | | Occipital Ctx | |
| AD 5 Sup Temporal | 28.5 | Control (Path) 3 | 1.4 |
| Ctx | | Occipital Ctx | |
| AD 6 Inf Temporal Ctx | 31.4 | Control (Path) 4 | 19.8 |
| | | Occipital Ctx | |
| AD 6 Sup Temporal | 33.7 | Control 1 Parietal Ctx | 2.4 |
| Ctx | | | |
| Control 1 Temporal | 1.8 | Control 2 Parietal Ctx | 31.0 |
| Ctx | | | |
| Control 2 Temporal | 41.2 | Control 3 Parietal Ctx | 21.2 |
| Ctx | | | |
| Control 3 Temporal | 16.6 | Control (Path) 1 | 94.6 |
| Ctx | | Parietal Ctx | |
| Control 3 Temporal | 4.3 | Control (Path) 2 | 22.7 |
| Ctx | | Parietal Ctx | |
| Control (Path) 1 | 72.7 | Control (Path) 3 | 2.2 |
| Temporal Ctx | | Parietal Ctx | |
| Control (Path) 2 | 40.6 | Control (Path) 4 | 47.6 |
| Temporal Ctx | | Parietal Ctx | |

TABLE XC
General_screening_panel_v1.4

| | Rel. Exp. (%) | | Rel. Exp. (%) |
|---|---|---|---|
| | Ag4339, Run | | Ag4339, Run |
| Tissue Name | 222523508 | Tissue Name | 222523508 |
| Adipose | 0.7 | Renal ca. TK-10 | 0.7 |
| Melanoma* | 0.9 | Bladder | 0.2 |
| Hs688(A).T | | | |
| Melanoma* | 0.8 | Gastric ca. (liver met.) | 0.1 |
| Hs688(B).T | | NCI-N87 | |
| Melanoma* M14 | 12.1 | Gastric ca. KATO III | 0.0 |
| Melanoma* LOXIMVI | 0.1 | Colon ca. SW-948 | 0.0 |
| Melanoma* SK-MEL-5 | 31.2 | Colon ca. SW480 | 3.5 |
| Squamous cell | 1.2 | Colon ca.* (SW480 met) | 0.4 |
| carcinoma SCC-4 | | SW620 | |
| Testis Pool | 0.4 | Colon ca. HT29 | 0.0 |
| Prostate ca.* (bone met) | 4.3 | Colon ca. HCT-116 | 1.4 |
| PC-3 | | | |
| Prostate Pool | 0.3 | Colon ca. CaCo-2 | 0.2 |
| Placenta | 0.8 | Colon cancer tissue | 0.4 |
| Uterus Pool | 0.3 | Colon ca. SW1116 | 0.7 |
| Ovarian ca. OVCAR-3 | 1.9 | Colon ca. Colo-205 | 0.1 |
| Ovarian ca. SK-OV-3 | 1.6 | Colon ca. SW-48 | 0.0 |
| Ovarian ca. OVCAR-4 | 0.7 | Colon Pool | 0.5 |
| Ovarian ca. OVCAR-5 | 1.1 | Small Intestine Pool | 1.6 |
| Ovarian ca. IGROV-1 | 1.3 | Stomach Pool | 0.3 |
| Ovarian ca. OVCAR-8 | 1.2 | Bone Marrow Pool | 0.3 |
| Ovary | 0.8 | Fetal Heart | 1.3 |
| Breast ca. MCF-7 | 0.1 | Heart Pool | 0.2 |
| Breast ca. MDA-MB- | 1.6 | Lymph Node Pool | 0.6 |
| 231 | | | |
| Breast ca. BT 549 | 5.3 | Fetal Skeletal Muscle | 1.3 |
| Breast ca. T47D | 1.7 | Skeletal Muscle Pool | 2.6 |
| Breast ca. MDA-N | 7.3 | Spleen Pool | 4.9 |
| Breast Pool | 0.6 | Thymus Pool | 0.8 |
| Trachea | 0.8 | CNS cancer (glio/astro) | 1.3 |
| | | U87-MG | |
| Lung | 0.2 | CNS cancer (glio/astro) U- | 1.5 |
| | | 118-MG | |
| Fetal Lung | 11.7 | CNS cancer (neuro; met) | 3.2 |
| | | SK-N-AS | |
| Lung ca. NCI-N417 | 0.7 | CNS cancer (astro) SF-539 | 0.6 |
| Lung ca. LX-1 | 0.2 | CNS cancer (astro) SNB- | 1.4 |
| | | 75 | |
| Lung ca. NCI-H146 | 2.1 | CNS cancer (glio) SNB-19 | 1.1 |
| Lung ca. SHP-77 | 3.0 | CNS cancer (glio) SF-295 | 2.9 |
| Lung ca. A549 | 0.7 | Brain (Amygdala) Pool | 13.4 |
| Lung ca. NCI-H526 | 0.9 | Brain (cerebellum) | 51.1 |
| Lung ca. NCI-H23 | 1.3 | Brain (fetal) | 20.3 |
| Lung ca. NCI-H460 | 0.0 | Brain (Hippocampus) Pool | 11.1 |
| Lung ca. HOP-62 | 0.6 | Cerebral Cortex Pool | 16.8 |
| Lung ca. NCI-H522 | 4.5 | Brain (Substantia nigra) | 13.8 |
| | | Pool | |
| Liver | 0.1 | Brain (Thalamus) Pool | 20.0 |
| Fetal Liver | 100.0 | Brain (whole) | 17.6 |
| Liver ca. HepG2 | 0.0 | Spinal Cord Pool | 15.7 |
| Kidney Pool | 1.8 | Adrenal Gland | 2.4 |
| Fetal Kidney | 1.4 | Pituitary gland Pool | 1.7 |
| Renal ca. 786-0 | 0.6 | Salivary Gland | 0.2 |
| Renal ca. A498 | 1.2 | Thyroid (female) | 0.4 |
| Renal ca. ACHN | 2.0 | Pancreatic ca. CAPAN2 | 0.1 |
| Renal ca. UO-31 | 0.8 | Pancreas Pool | 0.4 |

TABLE XD
Panel 4.1D

| | Rel. Exp. (%) | | Rel. Exp. (%) |
|---|---|---|---|
| | Ag4339, Run | | Ag4339, Run |
| Tissue Name | 184798183 | Tissue Name | 184798183 |
| Secondary Th1 act | 100.0 | HUVEC IL-1beta | 15.7 |
| Secondary Th2 act | 75.8 | HUVEC IFN gamma | 18.2 |
| Secondary Tr1 act | 59.0 | HUVEC TNF alpha + IFN | 53.2 |
| | | gamma | |
| Secondary Th1 rest | 23.0 | HUVEC TNF alpha + IL4 | 9.9 |
| Secondary Th2 rest | 9.1 | HUVEC IL-11 | 7.0 |
| Secondary Tr1 rest | 9.6 | Lung Microvascular EC none | 4.2 |
| Primary Th1 act | 92.0 | Lung Microvascular EC | 38.2 |
| | | TNF alpha + IL-1beta | |
| Primary Th2 act | 19.9 | Microvascular Dermal EC | 8.7 |
| | | none | |
| Primary Tr1 act | 33.0 | Microsvasular Dermal EC | 30.1 |
| | | TNF alpha + IL-1beta | |
| Primary Th1 rest | 3.1 | Bronchial epithelium | 11.7 |
| | | TNF alpha + IL1beta | |
| Primary Th2 rest | 1.9 | Small airway epithelium none | 2.7 |
| Primary Tr1 rest | 1.8 | Small airway epithelium | 1.2 |
| | | TNF alpha + IL-1beta | |
| CD45RA CD4 | 38.4 | Coronery artery SMC rest | 8.1 |
| lymphocyte act | | | |
| CD45RO CD4 | 72.2 | Coronery artery SMC | 13.3 |
| lymphocyte act | | TNF alpha + IL-1beta | |
| CD8 lymphocyte act | 29.7 | Astrocytes rest | 16.2 |
| Secondary CD8 | 18.3 | Astrocytes TNF alpha + IL- | 25.9 |
| lymphocyte rest | | 1beta | |
| Secondary CD8 | 16.7 | KU-812 (Basophil) rest | 47.0 |
| lymphocyte act | | | |
| CD4 lymphocyte none | 4.9 | KU-812 (Basophil) | 61.6 |
| | | PMA/ionomycin | |
| 2ry Th1/Th2/Tr1_anti- | 25.2 | CCD1106 (Keratinocytes) | 19.6 |
| CD95 CH11 | | none | |
| LAK cells rest | 3.1 | CCD1106 (Keratinocytes) | 12.7 |
| | | TNF alpha + IL-1beta | |
| LAK cells IL-2 | 12.9 | Liver cirrhosis | 1.9 |
| LAK cells IL-2 + IL-12 | 21.0 | NCI-H292 none | 5.0 |
| LAK cells IL-2 + IFN | 10.1 | NCI-H292 IL-4 | 11.8 |
| gamma | | | |
| LAK cells IL-2 + IL-18 | 14.2 | NCI-H292 IL-9 | 5.9 |
| LAK cells | 8.2 | NCI-H292 IL-13 | 7.4 |
| PMA/ionomycin | | | |
| NK Cells IL-2 rest | 14.3 | NCI-H292 IFN gamma | 2.2 |
| Two Way MLR 3 day | 13.7 | HPAEC none | 5.8 |
| Two Way MLR 5 day | 31.6 | HPAEC TNF alpha + IL-1beta | 35.4 |
| Two Way MLR 7 day | 14.4 | Lung fibroblast none | 25.0 |
| PBMC rest | 6.1 | Lung fibroblast TNF alpha + | 12.5 |
| | | IL-1beta | |
| PBMC PWM | 69.7 | Lung fibroblast IL-4 | 11.4 |
| PBMC PHA-L | 33.4 | Lung fibroblast IL-9 | 24.0 |
| Ramos (B cell) none | 8.8 | Lung fibroblast IL-13 | 10.7 |
| Ramos (B cell) ionomycin | 8.6 | Lung fibroblast IFN gamma | 2.5 |
| B lymphocytes PWM | 58.6 | Dermal fibroblast CCD1070 | 29.5 |
| | | rest | |
| B lymphocytes CD40L | 12.9 | Dermal fibroblast CCD1070 | 12.8 |
| and IL-4 | | TNF alpha | |
| EOL-1 dbcAMP | 6.5 | Dermal fibroblast CCD1070 | 16.6 |
| | | IL-1beta | |
| EOL-1 dbcAMP | 3.2 | Dermal fibroblast IFN gamma | 2.9 |
| PMA/ionomycin | | | |
| Dendritic cells none | 15.3 | Dermal fibroblast IL-4 | 12.7 |
| Dendritic cells LPS | 85.3 | Dermal Fibroblast rest | 30.4 |
| Dendritic cells anti-CD40 | 22.8 | Neutrophils TNF a + LPS | 0.0 |
| Monocytes rest | 0.0 | Neutrophils rest | 6.9 |
| Monocytes LPS | 31.4 | Colon | 1.9 |
| Macrophages rest | 11.0 | Lung | 8.4 |
| Macrophages LPS | 17.8 | Thymus | 13.8 |
| HUVEC none | 12.1 | Kidney | 16.2 |
| HUVEC starved | 10.0 | | |

CNS_neurodegeneration_v1.0 Summary: Ag4339 This panel confirms the expression of the CG107513-01 gene at low levels in the brains of an independent group of individuals.

However, no differential expression of this gene was detected between Alzheimer's diseased postmortem brains and those of non-demented controls in this experiment. Please see Panel 1.4 for a discussion of the potential use of this gene in treatment of central nervous system disorders.

General_screening_panel_v1.4 Summary: Ag4339 Highest expression of the CG107513-01 gene is detected in fetal liver (CT=24). Interestingly, this gene is expressed at much higher levels in fetal (CT=24) when compared to adult liver(CT=34.9). Thus expression of this gene can be used to distinguish fetal from adult liver and also from other samples used in this panel. In addition, the relative overexpression of this gene in fetal liver suggests that the protein product may enhance liver growth or development in the fetus and thus may also act in a regenerative capacity in the adult. Therefore, therapeutic modulation of the membrane protein encoded by this gene may be useful in treatment of liver related diseases.

Among tissues with metabolic or endocrine function, this gene is expressed at moderate levels in pancreas, adipose, adrenal gland, thyroid, pituitary gland, skeletal muscle, heart, liver and the gastrointestinal tract. Therefore, therapeutic modulation of the activity of this gene may prove useful in the treatment of endocrine/metabolically related diseases, such as obesity and diabetes.

In addition, this gene is expressed at high levels in all regions of the central nervous system examined, including amygdala, hippocampus, substantia nigra, thalamus, cerebellum, cerebral cortex, and spinal cord. Therefore, therapeutic modulation of the membrane protein encoded by this gene may be useful in the treatment of central nervous system disorders such as Alzheimer's disease, Parkinson's disease, epilepsy, multiple sclerosis, schizophrenia and depression.

Panel 4.1D Summary: Ag4339 Highest expression of the CG107513-01 gene is detected in activated secondary Th1 cells (CT=30.7). This gene is expressed at high to moderate levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. This pattern is in agreement with the expression profile in General_screening_panel_v1.4 and also suggests a role for the gene product in cell survival and proliferation. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

Y. CG107533-02: Tumor Necrosis Factor (Ligand) Superfamily, Member 7

Expression of gene CG107533-02 was assessed using the primer-probe set Ag6859, described in Table YA. Results of the RTQ-PCR runs are shown in Table YB.

TABLE YA

Probe Name Ag6859

| | | | Start | SEQ ID |
|---|---|---|---|---|
| Primers | Sequences | Length | Position | No |
| Forward | 5′-agtcacttggggacctcag-3′ | 19 | 191 | 237 |
| Probe | TET-5′-ctccttcctgcatggaccagagctg-3′-TAMRA | 25 | 254 | 238 |
| Reverse | 5′-catcacgatggatacgtagct-3′ | 21 | 289 | 239 |

TABLE YB
General_screening_panel_v1.6

| | Rel. Exp. (%) | | Rel. Exp. (%) |
|---|---|---|---|
| | Ag6859, Run | | Ag6859, Run |
| Tissue Name | 278387508 | Tissue Name | 278387508 |
| Adipose | 0.3 | Renal ca. TK-10 | 4.5 |
| Melanoma* | 0.0 | Bladder | 0.0 |
| Hs688(A).T | | | |
| Melanoma* | 0.0 | Gastric ca. (liver met.) | 0.0 |
| Hs688(B).T | | NCI-N87 | |
| Melanoma* M14 | 0.7 | Gastric ca. KATO III | 0.2 |
| Melanoma* LOXIMVI | 3.0 | Colon ca. SW-948 | 5.8 |
| Melanoma* SK-MEL-5 | 0.0 | Colon ca. SW480 | 2.5 |
| Squamous cell | 0.2 | Colon ca.* (SW480 met) | 0.0 |
| carcinoma SCC-4 | | SW620 | |
| Testis Pool | 0.0 | Colon ca. HT29 | 0.0 |
| Prostate ca.* (bone met) | 0.0 | Colon ca. HCT-116 | 0.1 |
| PC-3 | | | |
| Prostate Pool | 0.0 | Colon ca. CaCo-2 | 0.0 |
| Placenta | 0.0 | Colon cancer tissue | 0.5 |
| Uterus Pool | 0.0 | Colon ca. SW1116 | 0.0 |
| Ovarian ca. OVCAR-3 | 0.2 | Colon ca. Colo-205 | 0.0 |
| Ovarian ca. SK-OV-3 | 57.8 | Colon ca. SW-48 | 0.0 |
| Ovarian ca. OVCAR-4 | 0.1 | Colon Pool | 0.0 |
| Ovarian ca. OVCAR-5 | 0.0 | Small Intestine Pool | 0.2 |
| Ovarian ca. IGROV-1 | 2.4 | Stomach Pool | 0.0 |
| Ovarian ca. OVCAR-8 | 2.0 | Bone Marrow Pool | 0.0 |
| Ovary | 0.0 | Fetal Heart | 0.0 |
| Breast ca. MCF-7 | 0.0 | Heart Pool | 0.0 |
| Breast ca. MDA-MB- | 0.0 | Lymph Node Pool | 0.0 |
| 231 | | | |
| Breast ca. BT 549 | 100.0 | Fetal Skeletal Muscle | 0.0 |
| Breast ca. T47D | 0.0 | Skeletal Muscle Pool | 0.0 |
| Breast ca. MDA-N | 0.4 | Spleen Pool | 0.2 |
| Breast Pool | 0.0 | Thymus Pool | 0.0 |
| Trachea | 0.0 | CNS cancer (glio/astro) | 22.4 |
| | | U87-MG | |
| Lung | 0.1 | CNS cancer (glio/astro) U- | 21.5 |
| | | 118-MG | |
| Fetal Lung | 0.0 | CNS cancer (neuro; met) | 0.0 |
| | | SK-N-AS | |
| Lung ca. NCI-N417 | 0.0 | CNS cancer (astro) SF-539 | 1.0 |
| Lung ca. LX-1 | 0.0 | CNS cancer (astro) SNB- | 1.2 |
| | | 75 | |
| Lung ca. NCI-H146 | 0.0 | CNS cancer (glio) SNB-19 | 1.9 |
| Lung ca. SHP-77 | 0.0 | CNS cancer (glio) SF-295 | 7.1 |
| Lung ca. A549 | 0.3 | Brain (Amygdala) Pool | 0.0 |
| Lung ca. NCI-H526 | 1.3 | Brain (cerebellum) | 0.0 |
| Lung ca. NCI-H23 | 6.8 | Brain (fetal) | 0.0 |
| Lung ca. NCI-H460 | 13.6 | Brain (Hippocampus) Pool | 0.0 |
| Lung ca. HOP-62 | 0.1 | Cerebral Cortex Pool | 0.0 |
| Lung ca. NCI-H522 | 0.0 | Brain (Substantia nigra) | 0.4 |
| | | Pool | |
| Liver | 0.0 | Brain (Thalamus) Pool | 0.2 |
| Fetal Liver | 0.0 | Brain (whole) | 0.0 |
| Liver ca. HepG2 | 0.0 | Spinal Cord Pool | 0.0 |
| Kidney Pool | 0.0 | Adrenal Gland | 0.0 |
| Fetal Kidney | 0.0 | Pituitary gland Pool | 0.0 |
| Renal ca. 786-0 | 44.1 | Salivary Gland | 0.0 |
| Renal ca. A498 | 13.1 | Thyroid (female) | 0.0 |
| Renal ca. ACHN | 6.1 | Pancreatic ca. CAPAN2 | 0.0 |
| Renal ca. UO-31 | 3.8 | Pancreas Pool | 0.0 |

General_screening_panel_v1.6 Summary: Ag6859 Highest expression of the CG107533-02 gene is detected in breast cancer BT 549 cell line (CT=29.4). In addition, moderate to low levels of expression of this gene is also seen in number of cancer cell lines derived from colon, lung, renal, breast, ovarian, melanoma and brain cancers. Interestingly, expression of this gene is low or undectable in the samples derived from normal tissues. Thus, expression of this gene may be used to differentiate between these samples and other samples on this panel and as a marker to detect the presence of these cancers. Furthermore, therapeutic modulation of the expression or function of this gene may be effective in the treatment of colon, lung, renal, breast, ovarian, melanoma and brain cancers.

Z. CG107562-01 and CG107562-02: TM LRR Ig FnIII Domains

Expression of gene CG107562-01 and CG107562-02 was assessed using the primer-probe set Ag4340, described in Table ZA. Results of the RTQ-PCR runs are shown in Tables ZB, ZC, ZD and ZE.

TABLE ZA

Probe Name Ag4340

| | | | Start | SEQ ID |
|---|---|---|---|---|
| Primers | Sequences | Length | Position | No |
| Forward | 5′-agcttggtggacctgactctat-3′ | 22 | 605 | 240 |
| Probe | TET-5′-ttttattacacctcatgctttcgctg-3′-TAMRA | 26 | 643 | 241 |
| Reverse | 5′-aagccctcaaatttcgtaggt-3′ | 21 | 669 | 242 |

TABLE ZB
CNS_neurodegeneration_v1.0

| | Rel. Exp. (%) | Rel. Exp. (%) | | Rel. Exp. (%) | Rel. Exp. (%) |
|---|---|---|---|---|---|
| | Ag4340, Run | Ag4340, Run | | Ag4340, Run | Ag4340, Run |
| Tissue Name | 249266041 | 258587752 | Tissue Name | 249266041 | 258587752 |
| AD 1 Hippo | 4.4 | 3.7 | Control (Path) | 1.6 | 1.0 |
| | | | 3 Temporal | | |
| | | | Ctx | | |
| AD 2 Hippo | 14.0 | 10.4 | Control (Path) | 23.7 | 21.5 |
| | | | 4 Temporal | | |
| | | | Ctx | | |
| AD 3 Hippo | 2.4 | 1.6 | AD 1 | 10.4 | 7.5 |
| | | | Occipital Ctx | | |
| AD 4 Hippo | 1.6 | 1.7 | AD 2 | 0.0 | 0.0 |
| | | | Occipital Ctx | | |
| | | | (Missing) | | |
| AD 5 hippo | 100.0 | 100.0 | AD 3 | 2.0 | 1.8 |
| | | | Occipital Ctx | | |
| AD 6 Hippo | 28.1 | 24.7 | AD 4 | 8.7 | 7.1 |
| | | | Occipital Ctx | | |
| Control 2 Hippo | 15.3 | 9.4 | AD 5 | 18.2 | 30.6 |
| | | | Occipital Ctx | | |
| Control 4 Hippo | 1.0 | 0.9 | AD 6 | 45.1 | 25.5 |
| | | | Occipital Ctx | | |
| Control (Path) 3 | 1.0 | 1.1 | Control 1 | 0.9 | 0.4 |
| Hippo | | | Occipital Ctx | | |
| AD 1 Temporal | 3.1 | 3.3 | Control 2 | 73.2 | 52.1 |
| Ctx | | | Occipital Ctx | | |
| AD 2 Temporal | 22.2 | 18.0 | Control 3 | 8.8 | 10.4 |
| Ctx | | | Occipital Ctx | | |
| AD 3 Temporal | 0.8 | 1.7 | Control 4 | 0.5 | 0.8 |
| Ctx | | | Occipital Ctx | | |
| AD 4 Temporal | 8.8 | 8.6 | Control (Path) | 68.8 | 66.0 |
| Ctx | | | 1 Occipital | | |
| | | | Ctx | | |
| AD 5 Inf | 70.7 | 73.2 | Control (Path) | 8.5 | 9.6 |
| Temporal Ctx | | | 2 Occipital | | |
| | | | Ctx | | |
| AD 5 | 17.7 | 19.1 | Control (Path) | 0.8 | 0.4 |
| SupTemporal Ctx | | | 3 Occipital | | |
| | | | Ctx | | |
| AD 6 Inf | 37.1 | 27.0 | Control (Path) | 10.6 | 9.8 |
| Temporal Ctx | | | 4 Occipital | | |
| | | | Ctx | | |
| AD 6 Sup | 43.5 | 40.6 | Control 1 | 2.0 | 1.2 |
| Temporal Ctx | | | Parietal Ctx | | |
| Control 1 | 1.4 | 0.9 | Control 2 | 27.4 | 20.3 |
| Temporal Ctx | | | Parietal Ctx | | |
| Control 2 | 35.8 | 29.9 | Control 3 | 13.5 | 12.2 |
| Temporal Ctx | | | Parietal Ctx | | |
| Control 3 | 8.2 | 9.7 | Control (Path) | 71.2 | 70.7 |
| Temporal Ctx | | | 1 Parietal Ctx | | |
| Control 4 | 2.1 | 2.5 | Control (Path) | 17.4 | 15.1 |
| Temporal Ctx | | | 2 Parietal Ctx | | |
| Control (Path) 1 | 53.2 | 48.6 | Control (Path) | 1.1 | 0.8 |
| Temporal Ctx | | | 3 Parietal Ctx | | |
| Control (Path) 2 | 28.9 | 24.5 | Control (Path) | 33.9 | 35.6 |
| Temporal Ctx | | | 4 Parietal Ctx | | |

TABLE ZC
General_screening_panel_v1.4

| | Rel. Exp. (%) | | Rel. Exp. (%) |
|---|---|---|---|
| | Ag4340, Run | | Ag4340, Run |
| Tissue Name | 222523509 | Tissue Name | 222523509 |
| Adipose | 3.7 | Renal ca. TK-10 | 7.6 |
| Melanoma* | 0.6 | Bladder | 1.5 |
| Hs688(A).T | | | |
| Melanoma* | 0.3 | Gastric ca. (liver met.) | 0.0 |
| Hs688(B).T | | NCI-N87 | |
| Melanoma* M14 | 0.0 | Gastric ca. KATO III | 0.0 |
| Melanoma* LOXIMVI | 24.0 | Colon ca. SW-948 | 0.0 |
| Melanoma* SK-MEL-5 | 0.3 | Colon ca. SW480 | 0.0 |
| Squamous cell | 0.0 | Colon ca.* (SW480 met) | 0.0 |
| carcinoma SCC-4 | | SW620 | |
| Testis Pool | 4.9 | Colon ca. HT29 | 0.0 |
| Prostate ca.* (bone met) | 0.0 | Colon ca. HCT-116 | 0.0 |
| PC-3 | | | |
| Prostate Pool | 9.0 | Colon ca. CaCo-2 | 0.2 |
| Placenta | 1.0 | Colon cancer tissue | 1.5 |
| Uterus Pool | 3.8 | Colon ca. SW1116 | 0.0 |
| Ovarian ca. OVCAR-3 | 0.2 | Colon ca. Colo-205 | 0.0 |
| Ovarian ca. SK-OV-3 | 0.5 | Colon ca. SW-48 | 0.0 |
| Ovarian ca. OVCAR-4 | 0.0 | Colon Pool | 2.0 |
| Ovarian ca. OVCAR-5 | 0.0 | Small Intestine Pool | 12.4 |
| Ovarian ca. IGROV-1 | 0.2 | Stomach Pool | 9.3 |
| Ovarian ca. OVCAR-8 | 1.5 | Bone Marrow Pool | 11.3 |
| Ovary | 1.5 | Fetal Heart | 1.6 |
| Breast ca. MCF-7 | 8.8 | Heart Pool | 2.4 |
| Breast ca. MDA-MB- | 0.0 | Lymph Node Pool | 37.6 |
| 231 | | | |
| Breast ca. BT 549 | 2.4 | Fetal Skeletal Muscle | 4.8 |
| Breast ca. T47D | 0.0 | Skeletal Muscle Pool | 0.3 |
| Breast ca. MDA-N | 0.0 | Spleen Pool | 3.7 |
| Breast Pool | 9.9 | Thymus Pool | 14.2 |
| Trachea | 3.6 | CNS cancer (glio/astro) | 3.4 |
| | | U87-MG | |
| Lung | 1.8 | CNS cancer (glio/astro) U- | 76.8 |
| | | 118-MG | |
| Fetal Lung | 11.9 | CNS cancer (neuro; met) | 0.0 |
| | | SK-N-AS | |
| Lung ca. NCI-N417 | 7.7 | CNS cancer (astro) SF-539 | 0.0 |
| Lung ca. LX-1 | 0.0 | CNS cancer (astro) SNB- | 0.0 |
| | | 75 | |
| Lung ca. NCI-H146 | 4.2 | CNS cancer (glio) SNB-19 | 0.0 |
| Lung ca. SHP-77 | 1.8 | CNS cancer (glio) SF-295 | 8.8 |
| Lung ca. A549 | 0.0 | Brain (Amygdala) Pool | 22.1 |
| Lung ca. NCI-H526 | 0.0 | Brain (cerebellum) | 8.0 |
| Lung ca. NCI-H23 | 1.0 | Brain (fetal) | 100.0 |
| Lung ca. NCI-H460 | 3.3 | Brain (Hippocampus) Pool | 27.5 |
| Lung ca. HOP-62 | 0.8 | Cerebral Cortex Pool | 55.9 |
| Lung ca. NCI-H522 | 0.0 | Brain (Substantia nigra) | 34.4 |
| | | Pool | |
| Liver | 0.0 | Brain (Thalamus) Pool | 52.9 |
| Fetal Liver | 2.2 | Brain (whole) | 57.8 |
| Liver ca. HepG2 | 0.0 | Spinal Cord Pool | 5.6 |
| Kidney Pool | 7.5 | Adrenal Gland | 9.1 |
| Fetal Kidney | 12.2 | Pituitary gland Pool | 1.6 |
| Renal ca. 786-0 | 4.4 | Salivary Gland | 0.8 |
| Renal ca. A498 | 0.0 | Thyroid (female) | 1.3 |
| Renal ca. ACHN | 0.0 | Pancreatic ca. CAPAN2 | 0.0 |
| Renal ca. UO-31 | 0.0 | Pancreas Pool | 7.8 |

TABLE ZD
Panel 4.1D

| | Rel. Exp. (%) | | Rel. Exp. (%) |
|---|---|---|---|
| | Ag4340, Run | | Ag4340, Run |
| Tissue Name | 186362025 | Tissue Name | 186362025 |
| Secondary Th1 act | 0.0 | HUVEC IL-1beta | 0.6 |
| Secondary Th2 act | 0.0 | HUVEC IFN gamma | 0.0 |
| Secondary Tr1 act | 0.0 | HUVEC TNF alpha + IFN | 3.3 |
| | | gamma | |
| Secondary Th1 rest | 0.0 | HUVEC TNF alpha + IL4 | 0.0 |
| Secondary Th2 rest | 0.0 | HUVEC IL-11 | 0.5 |
| Secondary Tr1 rest | 0.0 | Lung Microvascular EC none | 0.0 |
| Primary Th1 act | 0.0 | Lung Microvascular EC | 5.2 |
| | | TNF alpha + IL-1beta | |
| Primary Th2 act | 0.0 | Microvascular Dermal EC | 0.0 |
| | | none | |
| Primary Tr1 act | 0.0 | Microsvasular Dermal EC | 0.0 |
| | | TNF alpha + IL-1beta | |
| Primary Th1 rest | 0.0 | Bronchial epithelium | 0.0 |
| | | TNF alpha + IL1beta | |
| Primary Th2 rest | 0.0 | Small airway epithelium none | 0.0 |
| Primary Tr1 rest | 0.0 | Small airway epithelium | 0.5 |
| | | TNF alpha + IL-1beta | |
| CD45RA CD4 | 2.7 | Coronery artery SMC rest | 0.0 |
| lymphocyte act | | | |
| CD45RO CD4 | 0.0 | Coronery artery SMC | 1.9 |
| lymphocyte act | | TNF alpha + IL-1beta | |
| CD8 lymphocyte act | 0.0 | Astrocytes rest | 0.0 |
| Secondary CD8 | 0.0 | Astrocytes TNF alpha + IL- | 9.2 |
| lymphocyte rest | | 1beta | |
| Secondary CD8 | 0.0 | KU-812 (Basophil) rest | 0.7 |
| lymphocyte act | | | |
| CD4 lymphocyte none | 1.0 | KU-812 (Basophil) | 3.8 |
| | | PMA/ionomycin | |
| 2ry Th1/Th2/Tr1_anti- | 0.0 | CCD1106 (Keratinocytes) | 0.0 |
| CD95 CH11 | | none | |
| LAK cells rest | 0.0 | CCD1106 (Keratinocytes) | 0.0 |
| | | TNF alpha + IL-1beta | |
| LAK cells IL-2 | 0.0 | Liver cirrhosis | 5.8 |
| LAK cells IL-2 + IL-12 | 0.0 | NCI-H292 none | 0.0 |
| LAK cells IL-2 + IFN | 0.0 | NCI-H292 IL-4 | 0.0 |
| gamma | | | |
| LAK cells IL-2 + IL-18 | 0.0 | NCI-H292 IL-9 | 0.0 |
| LAK cells | 0.0 | NCI-H292 IL-13 | 0.0 |
| PMA/ionomycin | | | |
| NK Cells IL-2 rest | 0.0 | NCI-H292 IFN gamma | 0.0 |
| Two Way MLR 3 day | 0.0 | HPAEC none | 1.2 |
| Two Way MLR 5 day | 0.0 | HPAEC TNF alpha + IL-1beta | 21.9 |
| Two Way MLR 7 day | 0.0 | Lung fibroblast none | 10.2 |
| PBMC rest | 0.0 | Lung fibroblast TNF alpha + | 11.5 |
| | | IL-1beta | |
| PBMC PWM | 0.0 | Lung fibroblast IL-4 | 46.7 |
| PBMC PHA-L | 0.0 | Lung fibroblast IL-9 | 48.3 |
| Ramos (B cell) none | 0.0 | Lung fibroblast IL-13 | 58.2 |
| Ramos (B cell) ionomycin | 0.0 | Lung fibroblast IFN gamma | 38.2 |
| B lymphocytes PWM | 0.0 | Dermal fibroblast CCD1070 | 8.0 |
| | | rest | |
| B lymphocytes CD40L | 1.3 | Dermal fibroblast CCD1070 | 19.5 |
| and IL-4 | | TNF alpha | |
| EOL-1 dbcAMP | 0.0 | Dermal fibroblast CCD1070 | 14.2 |
| | | IL-1beta | |
| EOL-1 dbcAMP | 0.0 | Dermal fibroblast IFN gamma | 3.7 |
| PMA/ionomycin | | | |
| Dendritic cells none | 0.0 | Dermal fibroblast IL-4 | 5.9 |
| Dendritic cells LPS | 0.0 | Dermal Fibroblasts rest | 8.1 |
| Dendritic cells anti-CD40 | 0.0 | Neutrophils TNF a + LPS | 1.3 |
| Monocytes rest | 0.0 | Neutrophils rest | 0.0 |
| Monocytes LPS | 0.0 | Colon | 1.0 |
| Macrophages rest | 0.0 | Lung | 10.2 |
| Macrophages LPS | 0.0 | Thymus | 22.1 |
| HUVEC none | 0.0 | Kidney | 100.0 |
| HUVEC starved | 0.0 | | |

TABLE ZE
general oncology screening panel_v_2.4

| | Rel. Exp. (%) | Rel. Exp. (%) | | Rel. Exp. (%) | Rel. Exp. (%) |
|---|---|---|---|---|---|
| | Ag4340, Run | Ag4340, Run | | Ag4340, Run | Ag4340, Run |
| Tissue Name | 258689189 | 260280474 | Tissue Name | 258689189 | 260280474 |
| Colon cancer 1 | 0.7 | 1.7 | Bladder cancer | 0.0 | 0.9 |
| | | | NAT 2 | | |
| Colon NAT 1 | 0.9 | 1.3 | Bladder cancer | 0.0 | 0.2 |
| | | | NAT 3 | | |
| Colon cancer 2 | 0.8 | 3.9 | Bladder cancer | 37.1 | 14.1 |
| | | | NAT 4 | | |
| Colon cancer | 1.3 | 1.2 | Adenocarcinoma of | 97.9 | 100.0 |
| NAT 2 | | | the prostate 1 | | |
| Colon cancer 3 | 9.0 | 2.4 | Adenocarcinoma of | 2.4 | 3.4 |
| | | | the prostate 2 | | |
| Colon cancer | 16.0 | 5.7 | Adenocarcinoma of | 14.2 | 5.5 |
| NAT 3 | | | the prostate 3 | | |
| Colon | 2.0 | 1.6 | Adenocarcinoma of | 12.5 | 5.1 |
| malignant | | | the prostate 4 | | |
| cancer 4 | | | | | |
| Colon normal | 1.7 | 1.2 | Prostate cancer | 2.9 | 0.8 |
| adjacent tissue 4 | | | NAT 5 | | |
| Lung cancer 1 | 1.1 | 0.7 | Adenocarcinoma of | 5.6 | 0.9 |
| | | | the prostate 6 | | |
| Lung NAT 1 | 0.0 | 0.7 | Adenocarcinoma of | 13.5 | 5.8 |
| | | | the prostate 7 | | |
| Lung cancer 2 | 100.0 | 51.1 | Adenocarcinoma of | 4.4 | 0.9 |
| | | | the prostate 8 | | |
| Lung NAT 2 | 1.7 | 0.8 | Adenocarcinoma of | 65.5 | 57.4 |
| | | | the prostate 9 | | |
| Squamous cell | 2.8 | 1.5 | Prostate cancer | 2.3 | 0.9 |
| carcinoma 3 | | | NAT 10 | | |
| Lung NAT 3 | 0.0 | 0.0 | Kidney cancer 1 | 1.5 | 1.2 |
| metastatic | 24.1 | 8.8 | KidneyNAT 1 | 2.4 | 0.8 |
| melanoma 1 | | | | | |
| Melanoma 2 | 2.4 | 0.9 | Kidney cancer 2 | 4.2 | 1.4 |
| Melanoma 3 | 3.4 | 0.9 | Kidney NAT 2 | 0.6 | 1.2 |
| metastatic | 46.7 | 11.1 | Kidney cancer 3 | 0.0 | 0.3 |
| melanoma 4 | | | | | |
| metastatic | 17.6 | 10.6 | Kidney NAT 3 | 0.0 | 0.8 |
| melanoma 5 | | | | | |
| Bladder cancer 1 | 3.3 | 4.2 | Kidney cancer 4 | 0.7 | 0.3 |
| Bladder cancer | 0.0 | 0.0 | Kidney NAT 4 | 2.0 | 0.9 |
| NAT 1 | | | | | |
| Bladder cancer 2 | 18.9 | 7.5 | | | |

CNS_neurodegeneration_v1.0 Summary: Ag4340 Two experiments with the same probe and primer set produce results that are in excellent agreement. Highest expression of this gene is seen in the hippocampus of a patient with Alzheimer's disease (CTs=30). The hippocampus is a critical brain region for the formation of long-term memory. This gene encodes a putative LRR/Ig/FNII containing protein. Fibronectin repeat regions are often involved in cell surface binding and in this protein may be involved in the formation and maintenance of specific neuronal networks in the brain. Therefore, this gene product is therefore an excellent drug target for the treatment of dementia (Alzheimer's, vascular, etc) or for memory enhancement.

General_screening_panel_v1.4 Summary: Ag4340 Highest expression of this gene is seen in the fetal brain (CT=28.7). In addition, this gene is expressed at moderate levels in thalamus, substantia nigra, cerebral cortex, hippocampus, and amygdala, with low, but significant expression in the cerebellum. This gene encodes a novel transmembrane protein that contains a putative leucine rich repeat region. Leucine rich repeats (LRR) mediate reversible protein-protein interactions and have diverse cellular functions, including cellular adhesion and signaling. Several of these proteins, such as connectin, slit, chaoptin, and Toll have pivotal roles in neuronal development in Drosophila and may play significant but distinct roles in neural development and in the adult nervous system of humans (Battye R. (2001) J. Neurosci. 21: 4290-4298. Itoh A. (1998) Brain Res. Mol. Brain Res. 62: 175-186). In Drosophilia, the LRR region of axon guidance proteins has been shown to be critical for their function (especially in axon repulsion). (Taniguchi H, Shishido E, Takeichi M, Nose A. (2000) J Neurobiol. 42:104-116.) Since the leucine-rich-repeat protein encoded by this gene shows high expression in the cerebral cortex, it is an excellent candidate neuronal guidance protein for axons, dendrites and/or growth cones in general. Therefore, therapeutic modulation of the levels of this protein, or possible signaling via this protein, may be of utility in enhancing/directing compensatory synaptogenesis and fiber growth in the CNS in response to neuronal death (stroke, head trauma), axon lesion (spinal cord injury), or neurodegeneration (Alzheimer's, Parkinson's, Huntington's, vascular dementia or any neurodegenerative disease).

Among tissues with metabolic function, this gene is expressed at low but significant levels in pituitary, adipose, adrenal gland, pancreas, fetal skeletal muscle, and adult and fetal heart, and liver. This widespread expression among these tissues suggests that this gene product may play a role in normal neuroendocrine and metabolic function and that disregulated expression of this gene may contribute to neuroendocrine disorders or metabolic diseases, such as obesity and diabetes.

Moderate levels of expression are also seen in colon cancer and some brain, breast, lung, renal, ovarian and melanoma cancer cell lines. Therefore, therapeutic modulation of this gene product may be useful in the treatment of these cancers.

Panel 4.1D Summary: Ag4340 Highest expression of this gene is seen in the kidney (CT=32). In addition, low but significant levels of expression are seen in activated lung and dermal fibroblasts, suggesting a role for this gene product in pathological and inflammatory conditions of the lung and skin.

general oncology screening panel_v_{—}2.4 Summary: Ag4340 Two experiments with the same probe and primer set produce results that are in excellent agreement. Highest expression of the gene is seen in prostate and lung cancer (CTs=29.3-30). In addition, this gene is more highly expressed in lung and prostate cancer than in the corresponding normal adjacent tissue. Thus, expression of this gene could be used as a marker of these cancers. Furthemore, therapeutic modulation of the expression or function of this gene product may be useful in the treatment of lung and prostate cancer.

AA. CG108184-01 and CG108184-02: Novel Transmembrane Protein Tm7

Expression of gene CG108184-01 and CG108184-02 was assessed using the primer-probe set Ag4350, described in Table AAA. Results of the RTQ-PCR runs are shown in Tables AAB, AAC and AAD. Please note that CG108184-02 represents a full-length physical clone of the CG108184-01 gene, validating the prediction of the gene sequence.

TABLE AAA

Probe Name Ag4350

| | | | Start | SEQ ID |
|---|---|---|---|---|
| Primers | Sequences | Length | Position | No |
| Forward | 5′-ggttgtctggcttttaccttct-3′ | 22 | 1089 | 243 |
| Probe | TET-5′-ttttccacaggatgcaccctggact-3′-TAMRA | 25 | 1126 | 244 |
| Reverse | 5′-CTGTCTTGGGTAGGAACTGATG-3′ | 22 | 1153 | 245 |

TABLE AAB
CNS_neurodegeneration_v1.0

| | Rel. Exp. (%) | | Rel. Exp. (%) |
|---|---|---|---|
| | Ag4350, Run | | Ag4350, |
| Tissue Name | 249266042 | Tissue Name | Run 249266042 |
| AD 1 Hippo | 11.0 | Control (Path) 3 | 5.2 |
| | | Temporal Ctx | |
| AD 2 Hippo | 22.4 | Control (Path) 4 | 24.3 |
| | | Temporal Ctx | |
| AD 3 Hippo | 6.3 | AD 1 Occipital Ctx | 6.3 |
| AD 4 Hippo | 5.7 | AD 2 Occipital Ctx | 0.0 |
| | | (Missing) | |
| AD 5 Hippo | 83.5 | AD 3 Occipital Ctx | 4.5 |
| AD 6 Hippo | 37.6 | AD 4 Occipital Ctx | 11.8 |
| Control 2 Hippo | 30.4 | AD 5 Occipital Ctx | 51.4 |
| Control 4 Hippo | 4.1 | AD 6 Occipital Ctx | 9.7 |
| Control (Path) 3 Hippo | 3.1 | Control 1 Occipital Ctx | 2.0 |
| AD 1 Temporal Ctx | 5.9 | Control 2 Occipital Ctx | 71.2 |
| AD 2 Temporal Ctx | 25.3 | Control 3 Occipital Ctx | 11.1 |
| AD 3 Temporal Ctx | 4.4 | Control 4 Occipital Ctx | 3.1 |
| AD 4 Temporal Ctx | 12.3 | Control (Path) 1 | 80.7 |
| | | Occipital Ctx | |
| AD 5 Inf Temporal Ctx | 94.6 | Control (Path) 2 | 6.6 |
| | | Occipital Ctx | |
| AD 5 Sup Temporal | 63.3 | Control (Path) 3 | 1.3 |
| Ctx | | Occipital Ctx | |
| AD 6 Inf Temporal Ctx | 23.3 | Control (Path) 4 | 7.7 |
| | | Occipital Ctx | |
| AD 6 Sup Temporal | 26.4 | Control 1 Parietal Ctx | 2.7 |
| Ctx | | | |
| Control 1 Temporal | 2.4 | Control 2 Parietal Ctx | 29.1 |
| Ctx | | | |
| Control 2 Temporal | 45.1 | Control 3 Parietal Ctx | 16.3 |
| Ctx | | | |
| Control 3 Temporal | 10.2 | Control (Path) 1 | 100.0 |
| Ctx | | Parietal Ctx | |
| Control 3 Temporal | 6.0 | Control (Path) 2 | 13.7 |
| Ctx | | Parietal Ctx | |
| Control (Path) 1 | 51.8 | Control (Path) 3 | 2.3 |
| Temporal Ctx | | Parietal Ctx | |
| Control (Path) 2 | 25.7 | Control (Path) 4 | 32.5 |
| Temporal Ctx | | Parietal Ctx | |

TABLE AAC
General_screening_panel_v1.4

| | Rel. Exp. (%) | | Rel. Exp. (%) |
|---|---|---|---|
| | Ag4350, | | Ag4350, |
| Tissue Name | Run 222523514 | Tissue Name | Run 222523514 |
| Adipose | 0.9 | Renal ca. TK-10 | 2.5 |
| Melanoma* | 0.0 | Bladder | 12.3 |
| Hs688(A).T | | | |
| Melanoma* | 0.1 | Gastric ca. (liver met.) | 8.5 |
| Hs688(B).T | | NCI-N87 | |
| Melanoma* M14 | 0.2 | Gastric ca. KATO III | 7.4 |
| Melanoma* LOXIMVI | 0.0 | Colon ca. SW-948 | 0.9 |
| Melanoma* SK-MEL-5 | 1.3 | Colon ca. SW480 | 0.8 |
| Squamous cell | 0.7 | Colon ca.* (SW480 met) | 0.8 |
| carcinoma SCC-4 | | SW620 | |
| Testis Pool | 2.2 | Colon ca. HT29 | 1.5 |
| Prostate ca.* (bone met) | 2.3 | Colon ca. HCT-116 | 4.0 |
| PC-3 | | | |
| Prostate Pool | 1.2 | Colon ca. CaCo-2 | 1.4 |
| Placenta | 2.6 | Colon cancer tissue | 2.1 |
| Uterus Pool | 1.1 | Colon ca. SW1116 | 1.5 |
| Ovarian ca. OVCAR-3 | 1.5 | Colon ca. Colo-205 | 0.7 |
| Ovarian ca. SK-OV-3 | 1.5 | Colon ca. SW-48 | 2.4 |
| Ovarian ca. OVCAR-4 | 0.1 | Colon Pool | 1.0 |
| Ovarian ca. OVCAR-5 | 6.1 | Small Intestine Pool | 2.6 |
| Ovarian ca. IGROV-1 | 1.2 | Stomach Pool | 20.7 |
| Ovarian ca. OVCAR-8 | 0.5 | Bone Marrow Pool | 1.1 |
| Ovary | 4.6 | Fetal Heart | 0.3 |
| Breast ca. MCF-7 | 1.9 | Heart Pool | 0.4 |
| Breast ca. MDA-MB- | 0.0 | Lymph Node Pool | 1.9 |
| 231 | | | |
| Breast ca. BT 549 | 0.9 | Fetal Skeletal Muscle | 0.3 |
| Breast ca. T47D | 9.0 | Skeletal Muscle Pool | 0.4 |
| Breast ca. MDA-N | 0.2 | Spleen Pool | 1.2 |
| Breast Pool | 1.2 | Thymus Pool | 1.1 |
| Trachea | 34.9 | CNS cancer (glio/astro) | 0.7 |
| | | U87-MG | |
| Lung | 1.5 | CNS cancer (glio/astro) U- | 0.0 |
| | | 118-MG | |
| Fetal Lung | 16.3 | CNS cancer (neuro; met) | 2.0 |
| | | SK-N-AS | |
| Lung ca. NCI-N417 | 2.0 | CNS cancer (astro) SF-539 | 0.0 |
| Lung ca. LX-1 | 1.5 | CNS cancer (astro) SNB- | 0.2 |
| | | 75 | |
| Lung ca. NCI-H146 | 0.3 | CNS cancer (glio) SNB-19 | 1.1 |
| Lung ca. SHP-77 | 1.4 | CNS cancer (glio) SF-295 | 0.4 |
| Lung ca. A549 | 2.9 | Brain (Amygdala) Pool | 74.7 |
| Lung ca. NCI-H526 | 1.1 | Brain (cerebellum) | 55.9 |
| Lung ca. NCI-H23 | 4.1 | Brain (fetal) | 10.5 |
| Lung ca. NCI-H460 | 0.4 | Brain (Hippocampus) Pool | 66.9 |
| Lung ca. HOP-62 | 0.1 | Cerebral Cortex Pool | 84.7 |
| Lung ca. NCI-H522 | 7.8 | Brain (Substantia nigra) | 81.2 |
| | | Pool | |
| Liver | 0.2 | Brain (Thalamus) Pool | 100.0 |
| Fetal Liver | 2.1 | Brain (whole) | 96.6 |
| Liver ca. HepG2 | 2.4 | Spinal Cord Pool | 7.9 |
| Kidney Pool | 3.7 | Adrenal Gland | 0.7 |
| Fetal Kidney | 10.4 | Pituitary gland Pool | 3.2 |
| Renal ca. 786-0 | 0.9 | Salivary Gland | 20.7 |
| Renal ca. A498 | 2.3 | Thyroid (female) | 1.9 |
| Renal ca. ACHN | 6.1 | Pancreatic ca. CAPAN2 | 2.7 |
| Renal ca. UO-31 | 5.5 | Pancreas Pool | 9.0 |

TABLE AAD
Panel 4.1D

| | Rel. Exp. (%) | | Rel. Exp. (%) |
|---|---|---|---|
| | Ag4350, | | Ag4350, |
| Tissue Name | Run 186362899 | Tissue Name | Run 186362899 |
| Secondary Th1 act | 0.2 | HUVEC IL-1beta | 0.2 |
| Secondary Th2 act | 0.4 | HUVEC IFN gamma | 0.0 |
| Secondary Tr1 act | 0.3 | HUVEC TNF alpha + IFN | 0.1 |
| | | gamma | |
| Secondary Th1 rest | 0.4 | HUVEC TNF alpha + IL4 | 0.0 |
| Secondary Th2 rest | 0.3 | HUVEC IL-11 | 0.2 |
| Secondary Tr1 rest | 0.3 | Lung Microvascular EC none | 0.2 |
| Primary Th1 act | 0.3 | Lung Microvascular EC | 0.2 |
| | | TNF alpha + IL-1beta | |
| Primary Th2 act | 0.7 | Microvascular Dermal EC | 1.5 |
| | | none | |
| Primary Tr1 act | 0.7 | Microsvasular Dermal EC | 0.1 |
| | | TNF alpha + IL-1beta | |
| Primary Th1 rest | 0.1 | Bronchial epithelium | 0.2 |
| | | TNF alpha + IL1beta | |
| Primary Th2 rest | 0.8 | Small airway epithelium none | 0.5 |
| Primary Tr1 rest | 0.2 | Small airway epithelium | 0.1 |
| | | TNF alpha + IL-1beta | |
| CD45RA CD4 | 1.2 | Coronery artery SMC rest | 0.6 |
| lymphocyte act | | | |
| CD45RO CD4 | 0.3 | Coronery artery SMC | 0.4 |
| lymphocyte act | | TNF alpha + IL-1beta | |
| CD8 lymphocyte act | 0.9 | Astrocytes rest | 0.0 |
| Secondary CD8 | 0.6 | Astrocytes TNF alpha + IL- | 0.7 |
| lymphocyte rest | | 1beta | |
| Secondary CD8 | 0.5 | KU-812 (Basophil) rest | 0.5 |
| lymphocyte act | | | |
| CD4 lymphocyte none | 0.1 | KU-812 (Basophil) | 0.3 |
| | | PMA/ionomycin | |
| 2ry Th1/Th2/Tr1_anti- | 1.9 | CCD1106 (Keratinocytes) | 0.0 |
| CD95 CH11 | | none | |
| LAK cells rest | 0.3 | CCD1106 (Keratinocytes) | 0.0 |
| | | TNF alpha + IL-1beta | |
| LAK cells IL-2 | 0.8 | Liver cirrhosis | 0.3 |
| LAK cells IL-2 + IL-12 | 0.6 | NCI-H292 none | 1.2 |
| LAK cells IL-2 + IFN | 0.6 | NCI-H292 IL-4 | 0.9 |
| gamma | | | |
| LAK cells IL-2 + IL-18 | 0.1 | NCI-H292 IL-9 | 2.9 |
| LAK cells | 0.2 | NCI-H292 IL-13 | 1.3 |
| PMA/ionomycin | | | |
| NK Cells IL-2 rest | 1.2 | NCI-H292 IFN gamma | 1.7 |
| Two Way MLR 3 day | 0.1 | HPAEC none | 0.4 |
| Two Way MLR 5 day | 0.7 | HPAEC TNF alpha + IL-1beta | 0.0 |
| Two Way MLR 7 day | 1.5 | Lung fibroblast none | 0.4 |
| PBMC rest | 0.0 | Lung fibroblast TNF alpha + | 0.4 |
| | | IL-1beta | |
| PBMC PWM | 0.7 | Lung fibroblast IL-4 | 0.2 |
| PBMC PHA-L | 0.4 | Lung fibroblast IL-9 | 0.0 |
| Ramos (B cell) none | 0.0 | Lung fibroblast IL-13 | 0.1 |
| Ramos (B cell) ionomycin | 0.9 | Lung fibroblast IFN gamma | 0.2 |
| B lymphocytes PWM | 0.5 | Dermal fibroblast CCD1070 | 0.2 |
| | | rest | |
| B lymphocytes CD40L | 0.3 | Dermal fibroblast CCD1070 | 0.6 |
| and IL-4 | | TNF alpha | |
| EOL-1 dbcAMP | 0.2 | Dermal fibroblast CCD1070 | 0.0 |
| | | IL-1beta | |
| EOL-1 dbcAMP | 0.0 | Dermal fibroblast IFN gamma | 0.3 |
| PMA/ionomycin | | | |
| Dendritic cells none | 0.2 | Dermal fibroblast IL-4 | 0.0 |
| Dendritic cells LPS | 0.0 | Dermal Fibroblasts rest | 0.3 |
| Dendritic cells anti-CD40 | 0.3 | Neutrophils TNFa + LPS | 0.0 |
| Monocytes rest | 0.2 | Neutrophils rest | 0.2 |
| Monocytes LPS | 0.0 | Colon | 2.3 |
| Macrophages rest | 0.4 | Lung | 5.5 |
| Macrophages LPS | 0.0 | Thymus | 4.9 |
| HUVEC none | 0.4 | Kidney | 100.0 |
| HUVEC starved | 0.1 | | |

CNS_neurodegeneration_v1.0 Summary: Ag4350 This panel confirms the expression of the CG108184-01 gene at low levels in the brains of an independent group of individuals. However, no differential expression of this gene was detected between Alzheimer's diseased postmortem brains and those of non-demented controls in this experiment. Please see Panel 1.4 for a discussion of the potential use of this gene in treatment of central nervous system disorders.

General_screening_panel_v1.4 Summary: Ag4350 Higest expression of the CG108184-01 gene is detected in brain (CTs=28.1). High expression of this gene is seen mainly in all the brain regions examined. Therefore, therapeutic modulation of this gene product may be useful in the treatment of central nervous system disorders such as Alzheimer's disease, Parkinson's disease, epilepsy, multiple sclerosis, schizophrenia and depression.

Moderate to low levels of expression of this gene is also seen in cluster of cancer cell lines derived from pancreas, gastric, colon, lung, renal, breast, ovarian, prostate, squamous cell carcinoma, melanoma and brain cancers. Thus, therapeutic modulation of the expression or function of this gene may be effective in the treatment of pancreas, gastric, colon, lung, renal, breast, ovarian, prostate, squamous cell carcinoma, melanoma and brain cancers.

Among tissues with metabolic or endocrine function, this gene is expressed at moderate to low levels in pancreas, adipose, thyroid, pituitary gland, fetal liver and the gastrointestinal tract. Therefore, therapeutic modulation of the activity of this gene may prove useful in the treatment of endocrine/metabolically related diseases, such as obesity and diabetes.

Interestingly, this gene is expressed at much higher levels in fetal (CTs=30.7-33.7) when compared to adult lung and liver (CTs=34-37). This observation suggests that expression of this gene can be used to distinguish fetal lung and liver from corresponding adult tissues. In addition, the relative overexpression of this gene in fetal tissue suggests that the protein product may enhance growth or development of lung and liver in the fetus and thus may also act in a regenerative capacity in the adult. Therefore, therapeutic modulation of the membrane protein encoded by this gene could be useful in treatment of lung and liver related diseases.

Panel 4.1D Summary: Ag4350 Higest expression of the CG108184-01 gene is detected in kidney (CT=27.8). Therefore, expression of this gene may be used to distinguish kidney from other samples used in this panel. Furthermore, therapeutic modulation of this gene product may be useful in the treatment of autoimmune and inflammatory disease that affect kidney, including lupus and glomerulonephritis.

In addition, moderate to low expression of this gene is also seen in CD45RA CD4 lymphocyte act, anti-CD95 CH11 treated secondary Th1/Th2/Tr1 cells, IL-2 treated LAK and NK cells, two way MLR, PWM treated PBMC, microvascular dermal EC, NCI-H292 and normal tissues represented by colon, lung and thymus. Therefore, therapeutic modulation of this gene product may be beneficial in the treatment of Crohn's disease, ulcerative colitis, multiple sclerosis, chronic obstructive pulmonary disease, asthma, emphysema, rheumatoid arthritis, lupus erythematosus, or psoriasis.

AB. CG108238-01: Sialic Acid Binding Immunoglobulin-like Lectin

Expression of gene CG108238-01 was assessed using the primer-probe set Ag4352, described in Table ABA. Results of the RTQ-PCR runs are shown in Tables ABB, ABC and ABD.

TABLE ABA

Probe Name Ag4352

| | | | Start | SEQ ID |
|---|---|---|---|---|
| Primers | Sequences | Length | Position | No |
| Forward | 5′-ggacccctgactgaatcct-3′ | 19 | 1278 | 246 |
| Probe | TET-5′-ctcccatggctgcctcctccttag-3′-TAMRA | 24 | 1324 | 247 |
| Reverse | 5′-atgctggagctctccttctc-3′ | 20 | 1348 | 248 |

TABLE ABB
CNS_neurodegeneration_v1.0

| | Rel. Exp. (%) | | Rel. Exp. (%) |
|---|---|---|---|
| | Ag4352, | | Ag4352, |
| Tissue Name | Run 224367442 | Tissue Name | Run 224367442 |
| AD 1 Hippo | 6.3 | Control (Path) 3 | 1.7 |
| | | Temporal Ctx | |
| AD 2 Hippo | 12.2 | Control (Path) 4 | 31.0 |
| | | Temporal Ctx | |
| AD 3 Hippo | 2.9 | AD 1 Occipital Ctx | 10.2 |
| AD 4 Hippo | 1.3 | AD 2 Occipital Ctx | 0.0 |
| | | (Missing) | |
| AD 5 hippo | 100.0 | AD 3 Occipital Ctx | 2.4 |
| AD 6 Hippo | 33.4 | AD 4 Occipital Ctx | 12.2 |
| Control 2 Hippo | 15.3 | AD 5 Occipital Ctx | 31.2 |
| Control 4 Hippo | 2.2 | AD 6 Occipital Ctx | 46.7 |
| Control (Path) 3 Hippo | 2.0 | Control 1 Occipital | 0.8 |
| | | Ctx | |
| AD 1 Temporal Ctx | 3.5 | Control 2 Occipital | 95.9 |
| | | Ctx | |
| AD 2 Temporal Ctx | 25.0 | Control 3 Occipital | 8.4 |
| | | Ctx | |
| AD 3 Temporal Ctx | 1.8 | Control 4 Occipital | 0.5 |
| | | Ctx | |
| AD 4 Temporal Ctx | 10.4 | Control (Path) 1 | 82.9 |
| | | Occipital Ctx | |
| AD 5 Inf Temporal Ctx | 88.3 | Control (Path) 2 | 10.5 |
| | | Occipital Ctx | |
| AD 5 SupTemporal Ctx | 22.2 | Control (Path) 3 | 0.4 |
| | | Occipital Ctx | |
| AD 6 Inf Temporal Ctx | 42.6 | Control (Path) 4 | 11.9 |
| | | Occipital Ctx | |
| AD 6 Sup Temporal Ctx | 57.4 | Control 1 Parietal Ctx | 3.2 |
| Control 1 Temporal Ctx | 0.9 | Control 2 Parietal Ctx | 31.0 |
| Control 2 Temporal Ctx | 41.8 | Control 3 Parietal Ctx | 15.1 |
| Control 3 Temporal Ctx | 9.0 | Control (Path) 1 | 95.3 |
| | | Parietal Ctx | |
| Control 4 Temporal Ctx | 4.0 | Control (Path) 2 | 18.2 |
| | | Parietal Ctx | |
| Control (Path) 1 | 59.0 | Control (Path) 3 | 1.8 |
| Temporal Ctx | | Parietal Ctx | |
| Control (Path) 2 | 29.9 | Control (Path) 4 | 36.3 |
| Temporal Ctx | | Parietal Ctx | |

TABLE ABC
General_screening_panel_v1.4

| | Rel. Exp. (%) | | Rel. Exp. (%) |
|---|---|---|---|
| | Ag4352, | | Ag4352, |
| Tissue Name | Run 222541798 | Tissue Name | Run 222541798 |
| Adipose | 6.8 | Renal ca. TK-10 | 0.0 |
| Melanoma* | 0.0 | Bladder | 7.7 |
| Hs688(A).T | | | |
| Melanoma* | 0.0 | Gastric ca. (liver met.) | 0.0 |
| Hs688(B).T | | NCI-N87 | |
| Melanoma* M14 | 0.0 | Gastric ca. KATO III | 0.0 |
| Melanoma* LOXIMVI | 0.0 | Colon ca. SW-948 | 0.0 |
| Melanoma* SK-MEL-5 | 0.0 | Colon ca. SW480 | 0.0 |
| Squamous cell | 0.0 | Colon ca.* (SW480 met) | 0.0 |
| carcinoma SCC-4 | | SW620 | |
| Testis Pool | 100.0 | Colon ca. HT29 | 0.0 |
| Prostate ca.* (bone met) | 0.0 | Colon ca. HCT-116 | 0.0 |
| PC-3 | | | |
| Prostate Pool | 9.7 | Colon ca. CaCo-2 | 0.0 |
| Placenta | 0.0 | Colon cancer tissue | 0.0 |
| Uterus Pool | 7.3 | Colon ca. SW1116 | 0.0 |
| Ovarian ca. OVCAR-3 | 0.0 | Colon ca. Colo-205 | 8.8 |
| Ovarian ca. SK-OV-3 | 0.0 | Colon ca. SW-48 | 0.0 |
| Ovarian ca. OVCAR-4 | 0.0 | Colon Pool | 4.4 |
| Ovarian ca. OVCAR-5 | 8.3 | Small Intestine Pool | 0.0 |
| Ovarian ca. IGROV-1 | 0.0 | Stomach Pool | 0.0 |
| Ovarian ca. OVCAR-8 | 0.0 | Bone Marrow Pool | 3.4 |
| Ovary | 0.0 | Fetal Heart | 10.2 |
| Breast ca. MCF-7 | 0.0 | Heart Pool | 7.7 |
| Breast ca. MDA-MB- | 0.0 | Lymph Node Pool | 14.6 |
| 231 | | | |
| Breast ca. BT 549 | 0.0 | Fetal Skeletal Muscle | 11.7 |
| Breast ca. T47D | 0.0 | Skeletal Muscle Pool | 0.0 |
| Breast ca. MDA-N | 0.0 | Spleen Pool | 23.3 |
| Breast Pool | 0.0 | Thymus Pool | 10.4 |
| Trachea | 5.3 | CNS cancer (glio/astro) | 0.0 |
| | | U87-MG | |
| Lung | 2.2 | CNS cancer (glio/astro) U- | 0.0 |
| | | 118-MG | |
| Fetal Lung | 4.5 | CNS cancer (neuro; met) | 0.0 |
| | | SK-N-AS | |
| Lung ca. NCI-N417 | 0.0 | CNS cancer (astro) SF-539 | 0.0 |
| Lung ca. LX-1 | 0.0 | CNS cancer (astro) SNB- | 0.0 |
| | | 75 | |
| Lung ca. NCI-H146 | 0.0 | CNS cancer (glio) SNB-19 | 0.0 |
| Lung ca. SHP-77 | 0.0 | CNS cancer (glio) SF-295 | 0.0 |
| Lung ca. A549 | 0.0 | Brain (Amygdala) Pool | 0.0 |
| Lung ca. NCI-H526 | 0.0 | Brain (cerebellum) | 0.0 |
| Lung ca. NCI-H23 | 15.8 | Brain (fetal) | 0.0 |
| Lung ca. NCI-H460 | 0.0 | Brain (Hippocampus) Pool | 0.0 |
| Lung ca. HOP-62 | 0.0 | Cerebral Cortex Pool | 0.0 |
| Lung ca. NCI-H522 | 0.0 | Brain (Substantia nigra) | 0.0 |
| | | Pool | |
| Liver | 0.0 | Brain (Thalamus) Pool | 0.0 |
| Fetal Liver | 0.0 | Brain (whole) | 0.0 |
| Liver ca. HepG2 | 0.0 | Spinal Cord Pool | 0.0 |
| Kidney Pool | 0.0 | Adrenal Gland | 0.0 |
| Fetal Kidney | 11.2 | Pituitary gland Pool | 0.0 |
| Renal ca. 786-0 | 33.4 | Salivary Gland | 0.0 |
| Renal ca. A498 | 0.0 | Thyroid (female) | 0.0 |
| Renal ca. ACHN | 0.0 | Pancreatic ca. CAPAN2 | 0.0 |
| Renal ca. UO-31 | 0.0 | Pancreas Pool | 10.6 |

TABLE ABD
Panel 4.1D

| | Rel. Exp. (%) | | Rel. Exp. (%) |
|---|---|---|---|
| | Ag4352, | | Ag4352, |
| Tissue Name | Run 186363976 | Tissue Name | Run 186363976 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC | 0.0 |
| | | TNF alpha + IL-1beta | |
| Primary Th2 act | 0.0 | Microvascular Dermal EC | 0.0 |
| | | none | |
| Primary Tr1 act | 0.0 | Microsvasular Dermal EC | 0.0 |
| | | TNF alpha + IL-1beta | |
| Primary Th1 rest | 0.0 | Bronchial epithelium | 0.0 |
| | | TNF alpha + IL1beta | |
| Primary Th2 rest | 0.0 | Small airway epithelium none | 0.0 |
| Primary Tr1 rest | 0.0 | Small airway epithelium | 0.0 |
| | | TNF alpha + IL-1beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNF alpha + IL-1beta | |
| CD8 lymphocyte act | 0.0 | Astrocytes rest | 0.0 |
| Secondary CD8 | 0.0 | Astrocytes TNF alpha + IL- | 0.0 |
| lymphocyte rest | | 1beta | |
| Secondary CD8 | 0.0 | KU-812 (Basophil) rest | 2.7 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CCD1106 (Keratinocytes) | 0.0 |
| CD95 CH11 | | none | |
| LAK cells rest | 0.0 | CCD1106 (Keratinocytes) | 0.0 |
| | | TNF alpha + IL-1beta | |
| LAK cells IL-2 | 0.0 | Liver cirrhosis | 0.0 |
| LAK cells IL-2 + IL-12 | 0.0 | NCI-H292 none | 0.0 |
| LAK cells IL-2 + IFN | 1.5 | NCI-H292 IL-4 | 0.0 |
| gamma | | | |
| LAK cells IL-2 + IL-18 | 0.0 | NCI-H292 IL-9 | 0.0 |
| LAK cells | 0.0 | NCI-H292 IL-13 | 0.0 |
| PMA/ionomycin | | | |
| NK Cells IL-2 rest | 0.0 | NCI-H292 IFN gamma | 0.0 |
| Two Way MLR 3 day | 0.0 | HPAEC none | 0.0 |
| Two Way MLR 5 day | 0.0 | HPAEC TNF alpha + IL-1beta | 0.0 |
| Two Way MLR 7 day | 0.0 | Lung fibroblast none | 0.0 |
| PBMC rest | 4.0 | Lung fibroblast TNF alpha + | 0.0 |
| | | IL-1beta | |
| PBMC PWM | 0.0 | Lung fibroblast IL-4 | 0.0 |
| PBMC PHA-L | 0.0 | Lung fibroblast IL-9 | 0.0 |
| Ramos (B cell) none | 0.0 | Lung fibroblast IL-13 | 7.6 |
| Ramos (B cell) ionomycin | 0.0 | Lung fibroblast IFN gamma | 1.9 |
| B lymphocytes PWM | 0.0 | Dermal fibroblast CCD1070 | 0.0 |
| | | rest | |
| B lymphocytes CD40L | 0.0 | Dermal fibroblast CCD1070 | 0.0 |
| and IL-4 | | TNF alpha | |
| EOL-1 dbcAMP | 1.6 | Dermal fibroblast CCD1070 | 0.0 |
| | | IL-1beta | |
| EOL-1 dbcAMP | 1.7 | Dermal fibroblast IFN gamma | 0.0 |
| PMA/ionomycin | | | |
| Dendritic cells none | 0.0 | Dermal fibroblast IL-4 | 0.0 |
| Dendritic cells LPS | 0.0 | Dermal Fibroblasts rest | 3.5 |
| Dendritic cells anti-CD40 | 0.0 | Neutrophils TNFa + LPS | 0.0 |
| Monocytes rest | 18.9 | Neutrophils rest | 0.0 |
| Monocytes LPS | 0.0 | Colon | 3.6 |
| Macrophages rest | 0.0 | Lung | 0.0 |
| Macrophages LPS | 0.0 | Thymus | 21.2 |
| HUVEC none | 0.0 | Kidney | 100.0 |
| HUVEC starved | 0.0 | | |

CNS_neurodegeneration_v1.0 Summary: Ag4352 This panel confirms the expression of the CG108238-01 gene at low levels in the brains of an independent group of individuals. However, no differential expression of this gene was detected between Alzheimer's diseased postmortem brains and those of non-demented controls in this experiment. Expression of this gene in brain suggests that this gene may play a role in central nervous system disorders such as Parkinson's disease, epilepsy, multiple sclerosis, schizophrenia and depression.

General_screening_panel_v1.4 Summary: Ag4352 Low levels of expression of the CG108238-01 gene is seen only in testis (CT=34.5). Therefore, expression of this gene may be used to distinguish testis from other samples used in the panel. In addition, therapeutic modulation of this gene product may a beneficial in the treatment of testis related diseases including fertility and hypogonadism.

Panel 4.1D Summary: Ag4352 Low levels of expression of the CG108238-01 gene is seen only in kidney (CT=33.6). Therefore, expression of this gene may be used to distinguish kidney from other samples used in the panel. In addition, therapeutic modulation of this gene product may a beneficial in the treatment of autoimmune and inflammatory diseases that affect kidney including lupus and glomerulonephritis.

AC. CG109505-01: Aldehyde Dehydrogenase

Expression of gene CG109505-01 was assessed using the primer-probe set Ag4387, described in Table ACA. Results of the RTQ-PCR runs are shown in Tables ACB and ACC.

TABLE ACA

Probe Name Ag4387

| | | | Start | SEQ ID |
|---|---|---|---|---|
| Primers | Sequences | Length | Position | No |
| Forward | 5′-tgtatccacagactgccagact-3′ | 22 | 744 | 249 |
| Probe | TET-5′-tcgtccgaaacatacagtcctttcaca-3′-TAMRA | 27 | 767 | 250 |
| Reverse | 5′-atgtcacaaaagttccgtgtgt-3′ | 22 | 797 | 252 |

TABLE ACB
General_screening_panel_v1.4

| | Rel. Exp. (%) | | Rel. Exp. (%) |
|---|---|---|---|
| | Ag4387, | | Ag4387, |
| Tissue Name | Run 222567011 | Tissue Name | Run 222567011 |
| Adipose | 0.5 | Renal ca. TK-10 | 0.0 |
| Melanoma* | 0.0 | Bladder | 7.8 |
| Hs688(A).T | | | |
| Melanoma* | 0.0 | Gastric ca. (liver met.) | 4.6 |
| Hs688(B).T | | NCI-N87 | |
| Melanoma* M14 | 0.0 | Gastric ca. KATO III | 0.0 |
| Melanoma* LOXIMVI | 0.0 | Colon ca. SW-948 | 0.0 |
| Melanoma* SK-MEL-5 | 0.0 | Colon ca. SW480 | 0.0 |
| Squamous cell | 4.4 | Colon ca.* (SW480 met) | 0.0 |
| carcinoma SCC-4 | | SW620 | |
| Testis Pool | 1.8 | Colon ca. HT29 | 0.0 |
| Prostate ca.* (bone met) | 0.0 | Colon ca. HCT-116 | 1.2 |
| PC-3 | | | |
| Prostate Pool | 0.0 | Colon ca. CaCo-2 | 0.0 |
| Placenta | 0.0 | Colon cancer tissue | 0.0 |
| Uterus Pool | 15.9 | Colon ca. SW1116 | 0.0 |
| Ovarian ca. OVCAR-3 | 2.8 | Colon ca. Colo-205 | 0.0 |
| Ovarian ca. SK-OV-3 | 0.0 | Colon ca. SW-48 | 0.0 |
| Ovarian ca. OVCAR-4 | 0.0 | Colon Pool | 0.0 |
| Ovarian ca. OVCAR-5 | 0.4 | Small Intestine Pool | 0.0 |
| Ovarian ca. IGROV-1 | 0.0 | Stomach Pool | 0.0 |
| Ovarian ca. OVCAR-8 | 0.0 | Bone Marrow Pool | 100.0 |
| Ovary | 0.0 | Fetal Heart | 0.0 |
| Breast ca. MCF-7 | 1.1 | Heart Pool | 0.0 |
| Breast ca. MDA-MB- | 0.0 | Lymph Node Pool | 0.0 |
| 231 | | | |
| Breast ca. BT 549 | 0.0 | Fetal Skeletal Muscle | 0.0 |
| Breast ca. T47D | 0.7 | Skeletal Muscle Pool | 0.0 |
| Breast ca. MDA-N | 0.0 | Spleen Pool | 0.0 |
| Breast Pool | 0.0 | Thymus Pool | 1.3 |
| Trachea | 10.1 | CNS cancer (glio/astro) | 0.0 |
| | | U87-MG | |
| Lung | 0.0 | CNS cancer (glio/astro) U- | 0.6 |
| | | 118-MG | |
| Fetal Lung | 0.4 | CNS cancer (neuro; met) | 0.0 |
| | | SK-N-AS | |
| Lung ca. NCI-N417 | 0.0 | CNS cancer (astro) SF-539 | 0.0 |
| Lung ca. LX-1 | 0.0 | CNS cancer (astro) SNB- | 0.0 |
| | | 75 | |
| Lung ca. NCI-H146 | 0.0 | CNS cancer (glio) SNB-19 | 0.0 |
| Lung ca. SHP-77 | 0.0 | CNS cancer (glio) SF-295 | 0.0 |
| Lung ca. A549 | 1.7 | Brain (Amygdala) Pool | 0.5 |
| Lung ca. NCI-H526 | 0.0 | Brain (cerebellum) | 0.0 |
| Lung ca. NCI-H23 | 1.5 | Brain (fetal) | 0.0 |
| Lung ca. NCI-H460 | 0.0 | Brain (Hippocampus) Pool | 0.0 |
| Lung ca. HOP-62 | 0.0 | Cerebral Cortex Pool | 0.0 |
| Lung ca. NCI-H522 | 0.0 | Brain (Substantia nigra) | 0.0 |
| | | Pool | |
| Liver | 0.0 | Brain (Thalamus) Pool | 0.0 |
| Fetal Liver | 0.0 | Brain (whole) | 0.0 |
| Liver ca. HepG2 | 0.0 | Spinal Cord Pool | 0.0 |
| Kidney Pool | 0.7 | Adrenal Gland | 0.0 |
| Fetal Kidney | 0.0 | Pituitary gland Pool | 0.0 |
| Renal ca. 786-0 | 0.5 | Salivary Gland | 2.4 |
| Renal ca. A498 | 0.0 | Thyroid (female) | 0.0 |
| Renal ca. ACHN | 0.0 | Pancreatic ca. CAPAN2 | 0.0 |
| Renal ca. UO-31 | 0.0 | Pancreas Pool | 0.0 |

TABLE ACC
Panel 4.1D

| | Rel. Exp. (%) | | Rel. Exp. (%) |
|---|---|---|---|
| | Ag4387, | | Ag4387, |
| Tissue Name | Run 186501500 | Tissue Name | Run 186501500 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.9 |
| Secondary Tr1 rest | 0.0 | Lung Microvascular EC none | 0.0 |
| Primary Th1 act | 0.0 | Lung Microvascular EC | 0.0 |
| | | TNF alpha + IL-1beta | |
| Primary Th2 act | 0.0 | Microvascular Dermal EC | 0.0 |
| | | none | |
| Primary Tr1 act | 0.0 | Microsvasular Dermal EC | 0.0 |
| | | TNF alpha + IL-1beta | |
| Primary Th1 rest | 0.3 | Bronchial epithelium | 4.6 |
| | | TNF alpha + IL1beta | |
| Primary Th2 rest | 0.0 | Small airway epithelium none | 20.0 |
| Primary Tr1 rest | 0.0 | Small airway epithelium | 22.4 |
| | | TNF alpha + IL-1beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNF alpha + IL-1beta | |
| CD8 lymphocyte act | 0.0 | Astrocytes rest | 0.0 |
| Secondary CD8 | 0.0 | Astrocytes TNF alpha + IL- | 0.0 |
| lymphocyte rest | | 1beta | |
| Secondary CD8 | 0.0 | KU-812 (Basophil) rest | 6.7 |
| lymphocyte act | | | |
| CD4 lymphocyte none | 0.0 | KU-812 (Basophil) | 16.3 |
| | | PMA/ionomycin | |
| 2ry Th1/Th2/Tr1_anti- | 0.0 | CCD1106 (Keratinocytes) | 0.4 |
| CD95 CH11 | | none | |
| LAK cells rest | 0.6 | CCD1106 (Keratinocytes) | 2.5 |
| | | TNF alpha + IL-1beta | |
| LAK cells IL-2 | 1.0 | Liver cirrhosis | 0.0 |
| LAK cells IL-2 + IL-12 | 0.9 | NCI-H292 none | 0.4 |
| LAK cells IL-2 + IFN | 0.0 | NCI-H292 IL-4 | 0.9 |
| gamma | | | |
| LAK cells IL-2 + IL-18 | 0.9 | NCI-H292 IL-9 | 0.0 |
| LAK cells | 0.6 | NCI-H292 IL-13 | 0.0 |
| PMA/ionomycin | | | |
| NK Cells IL-2 rest | 0.0 | NCI-H292 IFN gamma | 0.0 |
| Two Way MLR 3 day | 0.0 | HPAEC none | 0.0 |
| Two Way MLR 5 day | 0.0 | HPAEC TNF alpha + IL-1beta | 0.0 |
| Two Way MLR 7 day | 0.0 | Lung fibroblast none | 1.0 |
| PBMC rest | 0.0 | Lung fibroblast TNF alpha + | 0.4 |
| | | IL-1beta | |
| PBMC PWM | 0.9 | Lung fibroblast IL-4 | 0.0 |
| PBMC PHA-L | 0.0 | Lung fibroblast IL-9 | 0.0 |
| Ramos (B cell) none | 0.0 | Lung fibroblast IL-13 | 0.8 |
| Ramos (B cell) ionomycin | 0.0 | Lung fibroblast IFN gamma | 0.0 |
| B lymphocytes PWM | 0.5 | Dermal fibroblast CCD1070 | 0.0 |
| | | rest | |
| B lymphocytes CD40L | 0.0 | Dermal fibroblast CCD1070 | 0.0 |
| and IL-4 | | TNF alpha | |
| EOL-1 dbcAMP | 0.0 | Dermal fibroblast CCD1070 | 0.0 |
| | | IL-1beta | |
| EOL-1 dbcAMP | 0.0 | Dermal fibroblast IFN gamma | 0.0 |
| PMA/ionomycin | | | |
| Dendritic cells none | 0.0 | Dermal fibroblast IL-4 | 0.0 |
| Dendritic cells LPS | 0.0 | Dermal Fibroblasts rest | 0.0 |
| Dendritic cells anti-CD40 | 0.0 | Neutrophils TNFa + LPS | 0.0 |
| Monocytes rest | 0.0 | Neutrophils rest | 1.8 |
| Monocytes LPS | 0.0 | Colon | 2.0 |
| Macrophages rest | 0.0 | Lung | 2.1 |
| Macrophages LPS | 0.0 | Thymus | 18.4 |
| HUVEC none | 0.0 | Kidney | 100.0 |
| HUVEC starved | 0.0 | | |

General_screening_panel_v1.4 Summary: Ag4387 Highest expression of the CG109505-01 gene is detected in bone marrow (CT=30.6). Therefore, expression of this gene may be used to distinguish this sample from other samples used in this panel. In addition, therapeutic modulation of this gene product may be useful in the bone marrow related diseases such as leukemia.

Panel 4.1D Summary: Ag4387 Highest expression of the CG109505-01 gene is detected in kidney (CT=30.9). Therefore, expression of this gene may be used to distinguish kidney from other samples used in this panel. In addition, therapeutic modulation of this gene may be beneficial in the treatment of autoimmune of inflammatory disease that affect kidney including lupus and glomerulonephritis.

Moderate to low levels of expression of this gene is also seen in thymus, basophils, and small airway epithelium. Therefore, therapeutic modulation of this gene product may be beneficial in the treatment of asthma, allergies, COPD, and emphysema, inflammatory bowel disease, and autoimmune diseases.

AD. CG109742-01: Latent Transforming Growth Factor Beta Binding Protein 3 Like

Expression of gene CG109742-01 was assessed using the primer-probe sets Ag1112 and Ag25, described in Tables ADA. Results of the RTQ-PCR runs are shown in Table ADB.

TABLE ADA

Probe Name Ag25

| | | | Start | SEQ ID |
|---|---|---|---|---|
| Primers | Sequences | Length | Position | No |
| Forward | 5′-gtctgtgctgtggccgttct-3′ | 20 | 242 | 252 |
| Probe | TET-5′-cagcagggctccaacatgacgct-3′-TAMRA | 23 | 211 | 253 |
| Reverse | 5′-acctgtctcaagggccagtgt-3′ | 21 | 178 | 254 |

TABLE ADB
Panel 1

| | Rel. Exp. (%) | | Rel. Exp. (%) |
|---|---|---|---|
| | Ag25, Run | | Ag25, Run |
| Tissue Name | 91010022 | Tissue Name | 91010022 |
| Endothelial cells | 7.4 | Renal ca. 786-0 | 3.8 |
| Endothelial cells (treated) | 1.8 | Renal ca. A498 | 6.9 |
| Pancreas | 17.7 | Renal ca. RXF 393 | 30.1 |
| Pancreatic ca. CAPAN 2 | 9.6 | Renal ca. ACHN | 9.9 |
| Adrenal gland | 11.6 | Renal ca. UO-31 | 5.7 |
| Thyroid | 14.8 | Renal ca. TK-10 | 19.8 |
| Salivary gland | 6.7 | Liver | 34.4 |
| Pituitary gland | 7.7 | Liver (fetal) | 2.4 |
| Brain (fetal) | 5.3 | Liver ca. (hepatoblast) | 8.8 |
| | | HepG2 | |
| Brain (whole) | 19.2 | Lung | 15.0 |
| Brain (amygdala) | 7.2 | Lung (fetal) | 19.3 |
| Brain (cerebellum) | 52.9 | Lung ca. (small cell) LX-1 | 22.1 |
| Brain (hippocampus) | 7.6 | Lung ca. (small cell) | 0.8 |
| | | NCI-H69 | |
| Brain (substantia nigra) | 6.9 | Lung ca. (s. cell var.) | 1.8 |
| | | SHP-77 | |
| Brain (thalamus) | 6.8 | Lung ca. (large cell)NCI- | 13.3 |
| | | H460 | |
| Brain (hypothalamus) | 10.8 | Lung ca. (non-sm. cell) | 15.0 |
| | | A549 | |
| Spinal cord | 11.8 | Lung ca. (non-s. cell) | 4.1 |
| | | NCI-H23 | |
| glio/astro U87-MG | 7.1 | Lung ca. (non-s. cell) | 0.0 |
| | | HOP-62 | |
| glio/astro U-118-MG | 5.1 | Lung ca. (non-s. cl) NCI- | 1.6 |
| | | H522 | |
| astrocytoma SW1783 | 2.7 | Lung ca. (squam.) SW | 100.0 |
| | | 900 | |
| neuro*; met SK-N-AS | 2.6 | Lung ca. (squam.) NCI- | 0.4 |
| | | H596 | |
| astrocytoma SF-539 | 1.9 | Mammary gland | 50.3 |
| astrocytoma SNB-75 | 5.1 | Breast ca.* (pl. ef) MCF-7 | 7.4 |
| glioma SNB-19 | 10.4 | Breast ca.* (pl. ef) MDA- | 0.9 |
| | | MB-231 | |
| glioma U251 | 18.3 | Breast ca.* (pl. ef) T47D | 12.0 |
| glioma SF-295 | 27.5 | Breast ca. BT-549 | 16.4 |
| Heart | 20.4 | Breast ca. MDA-N | 3.7 |
| Skeletal muscle | 5.1 | Ovary | 59.0 |
| Bone marrow | 3.2 | Ovarian ca. OVCAR-3 | 3.5 |
| Thymus | 17.7 | Ovarian ca. OVCAR-4 | 6.3 |
| Spleen | 6.9 | Ovarian ca. OVCAR-5 | 49.0 |
| Lymph node | 16.8 | Ovarian ca. OVCAR-8 | 7.6 |
| Colon (ascending) | 3.6 | Ovarian ca. IGROV-1 | 1.1 |
| Stomach | 20.2 | Ovarian ca. (ascites) SK- | 1.2 |
| | | OV-3 | |
| Small intestine | 8.5 | Uterus | 9.9 |
| Colon ca. SW480 | 0.5 | Placenta | 21.0 |
| Colon ca.* SW620 | 1.9 | Prostate | 15.4 |
| (SW480 met) | | | |
| Colon ca. HT29 | 2.5 | Prostate ca.* (bone met) | 7.8 |
| | | PC-3 | |
| Colon ca. HCT-116 | 0.0 | Testis | 25.7 |
| Colon ca. CaCo-2 | 4.3 | Melanoma Hs688(A).T | 4.4 |
| Colon ca. HCT-15 | 3.8 | Melanoma* (met) | 14.6 |
| | | Hs688(B).T | |
| Colon ca. HCC-2998 | 0.8 | Melanoma UACC-62 | 3.3 |
| Gastric ca.* (liver met) | 39.8 | Melanoma M14 | 5.5 |
| NCI-N87 | | | |
| Bladder | 18.6 | Melanoma LOX IMVI | 90.8 |
| Trachea | 21.3 | Melanoma* (met) SK- | 0.0 |
| | | MEL-5 | |
| Kidney | 16.4 | Melanoma SK-MEL-28 | 3.2 |
| Kidney (fetal) | 23.3 | | |

Panel 1 Summary: Ag25 Highest expression of the CG109742-01 gene is detected in a lung cancer SW 900 cell line (CT=21.5). High expression of this gene is seen in cluster of cancer cell lines including melanoma, ovarian, breast, lung, renal, colon, gastric, pancreatic and CNS cancer cell lines. Therefore, therapeutic modulation of this gene product may be beneficial in the treatment of these cancers.

Among tissues with metabolic or endocrine function, this gene is expressed at high levels in pancreas, adrenal gland, thyroid, pituitary gland, skeletal muscle, heart, liver and the gastrointestinal tract. Therefore, therapeutic modulation of the activity of this gene may prove useful in the treatment of endocrine/metabolically related diseases, such as obesity and diabetes.

In addition, this gene is expressed at high levels in all regions of the central nervous system examined, including amygdala, hippocampus, substantia nigra, thalamus, cerebellum, cerebral cortex, and spinal cord. Therefore, this gene may play a role in central nervous system disorders such as Alzheimer's disease, Parkinson's disease, epilepsy, multiple sclerosis, schizophrenia and depression.

AE. CG109844-01: C4B-Binding Protein

Expression of gene CG109844-01 was assessed using the primer-probe sets Ag4406 and Ag4446, described in Tables AEA and AEB. Results of the RTQ-PCR runs are shown in Table AEC.

TABLE ABA

Probe Name Ag4406

| | | | Start | SEQ ID |
|---|---|---|---|---|
| Primers | Sequences | Length | Position | No |
| Forward | 5′-tatcctcagaggcagcagttta-3′ | 22 | 825 | 255 |
| Probe | TET-5′-tggtatccttctgttccctcttgcag-3′-TAMRA | 26 | 871 | 256 |
| Reverse | 5′-taggacagtgcaaccattcact-3′ | 22 | 897 | 257 |

TABLE AEB

Probe Name Ag4446

| | | | Start | SEQ ID |
|---|---|---|---|---|
| Primers | Sequences | Length | Position | No |
| Forward | 5′-tatcctcagaggcagcagttta-3′ | 22 | 825 | 258 |
| Probe | TET-5′-tggtatccttctgttccctcttgcag-3′-TAMRA | 26 | 871 | 259 |
| Reverse | 5′-taggacagtgcaaccattcact-3′ | 22 | 897 | 260 |

TABLE AEC
General_screening_panel_v1.4

| | Rel. Exp. (%) | | Rel. Exp. (%) |
|---|---|---|---|
| | Ag4446, | | Ag4446, |
| Tissue Name | Run 222693980 | Tissue Name | Run 222693980 |
| Adipose | 0.0 | Renal ca. TK-10 | 0.0 |
| Melanoma* | 0.0 | Bladder | 0.0 |
| Hs688(A).T | | | |
| Melanoma* | 0.0 | Gastric ca. (liver met.) | 0.0 |
| Hs688(B).T | | NCI-N87 | |
| Melanoma* M14 | 0.0 | Gastric ca. KATO III | 9.3 |
| Melanoma* LOXIMVI | 0.0 | Colon ca. SW-948 | 14.4 |
| Melanoma* SK-MEL-5 | 0.0 | Colon ca. SW480 | 0.0 |
| Squamous cell | 0.0 | Colon ca.* (SW480 met) | 0.0 |
| carcinoma SCC-4 | | SW620 | |
| Testis Pool | 34.6 | Colon ca. HT29 | 0.0 |
| Prostate ca.* (bone met) | 18.3 | Colon ca. HCT-116 | 100.0 |
| PC-3 | | | |
| Prostate Pool | 0.0 | Colon ca. CaCo-2 | 0.0 |
| Placenta | 0.0 | Colon cancer tissue | 0.0 |
| Uterus Pool | 0.0 | Colon ca. SW1116 | 0.0 |
| Ovarian ca. OVCAR-3 | 13.4 | Colon ca. Colo-205 | 0.0 |
| Ovarian ca. SK-OV-3 | 0.0 | Colon ca. SW-48 | 0.0 |
| Ovarian ca. OVCAR-4 | 0.0 | Colon Pool | 0.0 |
| Ovarian ca. OVCAR-5 | 0.0 | Small Intestine Pool | 0.0 |
| Ovarian ca. IGROV-1 | 0.0 | Stomach Pool | 0.0 |
| Ovarian ca. OVCAR-8 | 0.0 | Bone Marrow Pool | 0.0 |
| Ovary | 0.0 | Fetal Heart | 0.0 |
| Breast ca. MCF-7 | 0.0 | Heart Pool | 0.0 |
| Breast ca. MDA-MB- | 0.0 | Lymph Node Pool | 0.0 |
| 231 | | | |
| Breast ca. BT 549 | 0.0 | Fetal Skeletal Muscle | 0.0 |
| Breast ca. T47D | 11.2 | Skeletal Muscle Pool | 0.0 |
| Breast ca. MDA-N | 0.0 | Spleen Pool | 0.0 |
| Breast Pool | 0.0 | Thymus Pool | 0.0 |
| Trachea | 0.0 | CNS cancer (glio/astro) | 10.2 |
| | | U87-MG | |
| Lung | 0.0 | CNS cancer (glio/astro) U- | 56.3 |
| | | 118-MG | |
| Fetal Lung | 0.0 | CNS cancer (neuro; met) | 0.0 |
| | | SK-N-AS | |
| Lung ca. NCI-N417 | 0.0 | CNS cancer (astro) SF-539 | 0.0 |
| Lung ca. LX-1 | 0.0 | CNS cancer (astro) SNB- | 0.0 |
| | | 75 | |
| Lung ca. NCI-H146 | 0.0 | CNS cancer (glio) SNB-19 | 0.0 |
| Lung ca. SHP-77 | 0.0 | CNS cancer (glio) SF-295 | 0.0 |
| Lung ca. A549 | 0.0 | Brain (Amygdala) Pool | 0.0 |
| Lung ca. NCI-H526 | 0.0 | Brain (cerebellum) | 0.0 |
| Lung ca. NCI-H23 | 0.0 | Brain (fetal) | 0.0 |
| Lung ca. NCI-H460 | 0.0 | Brain (Hippocampus) Pool | 0.0 |
| Lung ca. HOP-62 | 0.0 | Cerebral Cortex Pool | 0.0 |
| Lung ca. NCI-H522 | 0.0 | Brain (Substantia nigra) | 0.0 |
| | | Pool | |
| Liver | 0.0 | Brain (Thalamus) Pool | 0.0 |
| Fetal Liver | 0.0 | Brain (whole) | 0.0 |
| Liver ca. HepG2 | 0.0 | Spinal Cord Pool | 0.0 |
| Kidney Pool | 0.0 | Adrenal Gland | 0.0 |
| Fetal Kidney | 0.0 | Pituitary gland Pool | 0.0 |
| Renal ca. 786-0 | 22.2 | Salivary Gland | 0.0 |
| Renal ca. A498 | 0.0 | Thyroid (female) | 0.0 |
| Renal ca. ACHN | 0.0 | Pancreatic ca. CAPAN2 | 0.0 |
| Renal ca. UO-31 | 0.0 | Pancreas Pool | 0.0 |

General_screening_panel_v1.4 Summary: Ag4406 Low levels of expression of the CG109844-01 gene is seen only in a colon cancer HCT-116 cell line (CT=34.9). Therefore, expression of this gene may be used to distinguish this sample from other samples used in this panel and also as diagnostic marker for colon cancer. In addition, therapeutic modulation of this gene product may be beneficial for the treatment of colon cancer.

AF. CG110014-03: Protein Tyrosine Kinase-7

Expression of gene CG110014-03 was assessed using the primer-probe set Ag6098, described in Table AFA. Results of the RTQ-PCR runs are shown in Table AFB.

TABLE AFA

Probe Name Ag6098

| | | | Start | SEQ ID |
|---|---|---|---|---|
| Primers | Sequences | Length | Position | No |
| Forward | 5′-gcaccctcgatgaaagct-3′ | 18 | 482 | 261 |
| Probe | TET-5′-atacctcgctactaccacgtcctggg-3′-TAMRA | 26 | 521 | 262 |
| Reverse | 5′-agaactggcaatggaacatg-3′ | 20 | 555 | 263 |

TABLE AFB
General_screening_panel_v1.5

| | Rel. Exp. (%) | | Rel. Exp. (%) |
|---|---|---|---|
| | Ag6098, | | Ag6098, |
| Tissue Name | Run 248491181 | Tissue Name | Run 248491181 |
| Adipose | 0.0 | Renal ca. TK-10 | 15.8 |
| Melanoma* | 46.7 | Bladder | 6.2 |
| Hs688(A).T | | | |
| Melanoma* | 19.6 | Gastric ca. (liver met.) | 22.7 |
| Hs688(B).T | | NCI-N87 | |
| Melanoma* M14 | 7.4 | Gastric ca. KATO III | 0.0 |
| Melanoma* LOXIMVI | 2.7 | Colon ca. SW-948 | 0.0 |
| Melanoma* SK-MEL-5 | 5.8 | Colon ca. SW480 | 20.7 |
| Squamous cell | 20.4 | Colon ca.* (SW480 met) | 39.5 |
| carcinoma SCC-4 | | SW620 | |
| Testis Pool | 5.6 | Colon ca. HT29 | 3.8 |
| Prostate ca.* (bone met) | 10.7 | Colon ca. HCT-116 | 100.0 |
| PC-3 | | | |
| Prostate Pool | 0.0 | Colon ca. CaCo-2 | 26.8 |
| Placenta | 21.5 | Colon cancer tissue | 10.0 |
| Uterus Pool | 5.7 | Colon ca. SW1116 | 16.8 |
| Ovarian ca. OVCAR-3 | 25.9 | Colon ca. Colo-205 | 4.8 |
| Ovarian ca. SK-OV-3 | 25.7 | Colon ca. SW-48 | 0.0 |
| Ovarian ca. OVCAR-4 | 9.9 | Colon Pool | 6.0 |
| Ovarian ca. OVCAR-5 | 8.6 | Small Intestine Pool | 9.3 |
| Ovarian ca. IGROV-1 | 11.0 | Stomach Pool | 4.3 |
| Ovarian ca. OVCAR-8 | 6.8 | Bone Marrow Pool | 2.2 |
| Ovary | 19.1 | Fetal Heart | 1.3 |
| Breast ca. MCF-7 | 3.4 | Heart Pool | 9.1 |
| Breast ca. MDA-MB- | 10.0 | Lymph Node Pool | 10.8 |
| 231 | | | |
| Breast ca. BT 549 | 46.7 | Fetal Skeletal Muscle | 12.2 |
| Breast ca. T47D | 2.7 | Skeletal Muscle Pool | 0.0 |
| Breast ca. MDA-N | 5.8 | Spleen Pool | 5.2 |
| Breast Pool | 13.6 | Thymus Pool | 29.3 |
| Trachea | 1.6 | CNS cancer (glio/astro) | 0.8 |
| | | U87-MG | |
| Lung | 0.0 | CNS cancer (glio/astro) U- | 0.0 |
| | | 118-MG | |
| Fetal Lung | 35.8 | CNS cancer (neuro; met) | 20.0 |
| | | SK-N-AS | |
| Lung ca. NCI-N417 | 4.5 | CNS cancer (astro) SF-539 | 80.7 |
| Lung ca. LX-1 | 40.1 | CNS cancer (astro) SNB- | 39.5 |
| | | 75 | |
| Lung ca. NCI-H146 | 5.3 | CNS cancer (glio) SNB-19 | 3.7 |
| Lung ca. SHP-77 | 24.3 | CNS cancer (glio) SF-295 | 49.7 |
| Lung ca. A549 | 0.0 | Brain (Amygdala) Pool | 0.0 |
| Lung ca. NCI-H526 | 6.3 | Brain (cerebellum) | 32.5 |
| Lung ca. NCI-H23 | 27.5 | Brain (fetal) | 7.3 |
| Lung ca. NCI-H460 | 0.0 | Brain (Hippocampus) Pool | 0.0 |
| Lung ca. HOP-62 | 8.0 | Cerebral Cortex Pool | 0.0 |
| Lung ca. NCI-H522 | 7.8 | Brain (Substantia nigra) | 1.1 |
| | | Pool | |
| Liver | 0.0 | Brain (Thalamus) Pool | 2.8 |
| Fetal Liver | 1.1 | Brain (whole) | 5.3 |
| Liver ca. HepG2 | 34.6 | Spinal Cord Pool | 57.0 |
| Kidney Pool | 13.2 | Adrenal Gland | 0.7 |
| Fetal Kidney | 39.8 | Pituitary gland Pool | 2.7 |
| Renal ca. 786-0 | 0.0 | Salivary Gland | 1.4 |
| Renal ca. A498 | 0.0 | Thyroid (female) | 0.7 |
| Renal ca. ACHN | 4.6 | Pancreatic ca. CAPAN2 | 0.0 |
| Renal ca. UO-31 | 7.0 | Pancreas Pool | 6.0 |

General_screening_panel_v1.5 Summary: Ag6098 Highest expression of the CG110014-03 gene is detected in colon cancer HCT-116 cell line (CT=32.9). In addition, low to moderate expression of this gene is also seen in number of cancer cell lines including CNS, colon, liver, lung, breast, ovarain and melanoma cancer cell lines. This gene codes for a splice variant of tyrosine protein kinase-like 7 precursor (colon carcinoma kinase 4, CCK-4; PTK7), belonging to protein-tyrosine kinases (PTKs) family. PTKs play important role in regulating cell proliferation and differentiation during development. Mossie et al. (1995, Oncogene 11(10):2179-84, PMID: 7478540) showed a varied expression of CCK-4 in colon cancer cell lines and suggested a tumor-characteristic role for CCK-4 as a signal amplifier or modulator. Therefore, therapeutic modulation of this gene product may be beneficial in the treatment of melanoma, CNS, colon, liver, lung, breast, and ovarian cancers.

Moderate expression of this gene is also seen in spinal cord sample. Therefore, therapeutic modulation of this gene product may be useful in the treatment of spinal cord related diseases.

Low expression of this gene is also detected in fetal lung. Interestingly, expression of this gene is higher in fetal (CT=34.3) as compared to adult lung (CT=40). Therefore, the expression of this gene may be used to distinguish the fetal from adult lung. In addition, the relative overexpression of this gene in fetal lung suggests that the protein product may enhance lung growth or development in the fetus and thus may also act in a regenerative capacity in the adult. Therefore, therapeutic modulation of the CCK-4 protein encoded by this gene could be useful in treatment of lung related diseases.

AG. CG110014-04: Protein Tyrosine Kinase-7

Expression of gene CG110014-04 was assessed using the primer-probe set Ag6687, described in Table AGA. Results of the RTQ-PCR runs are shown in Table AGB.

TABLE AGA

Probe Name Ag6687

| | | | Start | SEQ ID |
|---|---|---|---|---|
| Primers | Sequences | Length | Position | No |
| Forward | 5′-cacggttcgaggtgttcct-3′ | 19 | 1395 | 264 |
| Probe | TET-5′-tacttgtgaagagcctgcagagcaaggat-3′-TAMRA | 29 | 1458 | 265 |
| Reverse | 5′-tccacatattccagcaccatg-3′ | 21 | 1594 | 266 |

TABLE AGB
General_screening_panel_v1.6

| | Rel. Exp. (%) | | Rel. Exp. (%) |
|---|---|---|---|
| | Ag6687, | | Ag6687, |
| Tissue Name | Run 277259294 | Tissue Name | Run 277259294 |
| Adipose | 0.0 | Renal ca. TK-10 | 30.6 |
| Melanoma* | 73.7 | Bladder | 4.7 |
| Hs688(A).T | | | |
| Melanoma* | 63.7 | Gastric ca. (liver met.) | 28.5 |
| Hs688(B).T | | NCI-N87 | |
| Melanoma* M14 | 20.7 | Gastric ca. KATO III | 0.0 |
| Melanoma* LOXIMVI | 3.3 | Colon ca. SW-948 | 0.0 |
| Melanoma* SK-MEL-5 | 2.4 | Colon ca. SW480 | 34.9 |
| Squamous cell | 8.7 | Colon ca.* (SW480 met) | 36.3 |
| carcinoma SCC-4 | | SW620 | |
| Testis Pool | 1.4 | Colon ca. HT29 | 9.3 |
| Prostate ca.* (bone met) | 12.2 | Colon ca. HCT-116 | 33.2 |
| PC-3 | | | |
| Prostate Pool | 2.7 | Colon ca. CaCo-2 | 27.5 |
| Placenta | 6.2 | Colon cancer tissue | 14.0 |
| Uterus Pool | 0.7 | Colon ca. SW1116 | 37.6 |
| Ovarian ca. OVCAR-3 | 27.9 | Colon ca. Colo-205 | 0.8 |
| Ovarian ca. SK-OV-3 | 55.1 | Colon ca. SW-48 | 0.0 |
| Ovarian ca. OVCAR-4 | 21.2 | Colon Pool | 10.2 |
| Ovarian ca. OVCAR-5 | 2.7 | Small Intestine Pool | 7.1 |
| Ovarian ca. IGROV-1 | 13.7 | Stomach Pool | 8.4 |
| Ovarian ca. OVCAR-8 | 19.5 | Bone Marrow Pool | 1.1 |
| Ovary | 15.5 | Fetal Heart | 0.6 |
| Breast ca. MCF-7 | 5.9 | Heart Pool | 5.0 |
| Breast ca. MDA-MB- | 26.1 | Lymph Node Pool | 12.8 |
| 231 | | | |
| Breast ca. BT 549 | 71.7 | Fetal Skeletal Muscle | 0.6 |
| Breast ca. T47D | 8.0 | Skeletal Muscle Pool | 0.0 |
| Breast ca. MDA-N | 0.0 | Spleen Pool | 4.0 |
| Breast Pool | 12.6 | Thymus Pool | 18.8 |
| Trachea | 7.3 | CNS cancer (glio/astro) | 4.5 |
| | | U87-MG | |
| Lung | 2.3 | CNS cancer (glio/astro) U- | 3.5 |
| | | 118-MG | |
| Fetal Lung | 22.8 | CNS cancer (neuro; met) | 33.4 |
| | | SK-N-AS | |
| Lung ca. NCI-N417 | 3.0 | CNS cancer (astro) SF-539 | 64.6 |
| Lung ca. LX-1 | 59.9 | CNS cancer (astro) SNB- | 100.0 |
| | | 75 | |
| Lung ca. NCI-H146 | 9.9 | CNS cancer (glio) SNB-19 | 22.4 |
| Lung ca. SHP-77 | 8.9 | CNS cancer (glio) SF-295 | 82.9 |
| Lung ca. A549 | 2.1 | Brain (Amygdala) Pool | 0.0 |
| Lung ca. NCI-H526 | 6.8 | Brain (cerebellum) | 1.7 |
| Lung ca. NCI-H23 | 16.5 | Brain (fetal) | 3.3 |
| Lung ca. NCI-H460 | 0.0 | Brain (Hippocampus) Pool | 0.0 |
| Lung ca. HOP-62 | 17.6 | Cerebral Cortex Pool | 0.0 |
| Lung ca. NCI-H522 | 15.4 | Brain (Substantia nigra) | 0.0 |
| | | Pool | |
| Liver | 0.0 | Brain (Thalamus) Pool | 0.0 |
| Fetal Liver | 1.1 | Brain (whole) | 2.2 |
| Liver ca. HepG2 | 12.2 | Spinal Cord Pool | 0.0 |
| Kidney Pool | 22.7 | Adrenal Gland | 0.0 |
| Fetal Kidney | 13.0 | Pituitary gland Pool | 0.0 |
| Renal ca. 786-0 | 0.0 | Salivary Gland | 3.8 |
| Renal ca. A498 | 0.0 | Thyroid (female) | 5.6 |
| Renal ca. ACHN | 14.1 | Pancreatic ca. CAPAN2 | 4.8 |
| Renal ca. UO-31 | 26.2 | Pancreas Pool | 0.0 |

General_screening_panel_v1.6 Summary: Ag6687 Highest expression of the CG110014-04 gene is detected in CNS cancer cell line SNB-75 (CT=32.6). In addition, moderate to low levels of expression of this gene is also seen in number of cancer cell lines derived from gastric, colon, lung, renal, breast, ovarian, melanoma and brain cancers. Thus, expression of this gene could be used to differentiate between these samples and other samples on this panel and as a marker to detect the presence of these cancers. Furthermore, therapeutic modulation of the expression or function of this gene may be effective in the treatment of gastric, colon, lung, renal, breast, ovarian, melanoma and brain cancers.

Low levels of expression of this gene is also seen in kidney and fetal lung. Interestingly, this gene is expressed at much higher levels in fetal (CT=34.7) when compared to adult lung (CT=38). This observation suggests that expression of this gene can be used to distinguish fetal from adult lung. In addition, the relative overexpression of this gene in fetal lung suggests that the protein product may enhance lung growth or development in the fetus and thus may also act in a regenerative capacity in the adult. Therefore, therapeutic modulation of the protein encoded by this gene could be useful in treatment of lung and kidney related diseases.

AH. CG110187-01: Novel Alpha C1-like Protocadherin

Expression of gene CG110187-01 was assessed using the primer-probe set Ag4412, described in Table AHA. Results of the RTQ-PCR runs are shown in Tables AHB, AHC, AHD, AHE and AHF.

TABLE AHA
Probe Name Ag4412

| | | | Start | SEQ ID |
|---|---|---|---|---|
| Primers | Sequences | Length | Position | No. |
| Forward | 5′-gctttctgcccagaacttgtat-3′ | 22 | 2029 | 267 |
| Probe | TET-5′-aattgccttggcttgtatttcctttt-3′-TAMRA | 26 | 2056 | 268 |
| Reverse | 5′-aagaaaagtaagcaccccagaa-3′ | 22 | 2085 | 269 |

TABLE AHB
CNS_neurodegeneration_v1.0

| | Rel. Exp. (%) | | Rel. Exp. (%) |
|---|---|---|---|
| | Ag4412, Run | | Ag4412, |
| Tissue Name | 224505948 | Tissue Name | Run 224505948 |
| AD 1 Hippo | 8.1 | Control (Path) 3 | 2.0 |
| | | Temporal Ctx | |
| AD 2 Hippo | 11.9 | Control (Path) 4 | 21.3 |
| | | Temporal Ctx | |
| AD 3 Hippo | 4.9 | AD 1 Occipital Ctx | 10.2 |
| AD 4 Hippo | 1.2 | AD 2 Occipital Ctx | 0.0 |
| | | (Missing) | |
| AD 5 Hippo | 76.8 | AD 3 Occipital Ctx | 4.2 |
| AD 6 Hippo | 39.8 | AD 4 Occipital Ctx | 11.3 |
| Control 2 Hippo | 11.2 | AD 5 Occipital Ctx | 29.9 |
| Control 4 Hippo | 4.6 | AD 6 Occipital Ctx | 12.6 |
| Control (Path) 3 Hippo | 1.9 | Control 1 Occipital Ctx | 1.6 |
| AD 1 Temporal Ctx | 4.4 | Control 2 Occipital Ctx | 65.1 |
| AD 2 Temporal Ctx | 23.2 | Control 3 Occipital Ctx | 7.5 |
| AD 3 Temporal Ctx | 2.7 | Control 4 Occipital Ctx | 0.0 |
| AD 4 Temporal Ctx | 10.7 | Control (Path) 1 | 73.7 |
| | | Occipital Ctx | |
| AD 5 Inf Temporal Ctx | 66.0 | Control (Path) 2 | 15.5 |
| | | Occipital Ctx | |
| AD 5 Sup Temporal | 14.3 | Control (Path) 3 | 1.6 |
| Ctx | | Occipital Ctx | |
| AD 6 Inf Temporal Ctx | 33.7 | Control (Path) 4 | 9.2 |
| | | Occipital Ctx | |
| AD 6 Sup Temporal | 36.3 | Control 1 Parietal Ctx | 3.8 |
| Ctx | | | |
| Control 1 Temporal | 4.6 | Control 2 Parietal Ctx | 26.2 |
| Ctx | | | |
| Control 2 Temporal | 36.3 | Control 3 Parietal Ctx | 12.7 |
| Ctx | | | |
| Control 3 Temporal | 12.0 | Control (Path) 1 | 100.0 |
| Ctx | | Parietal Ctx | |
| Control 3 Temporal | 2.1 | Control (Path) 2 | 22.8 |
| Ctx | | Parietal Ctx | |
| Control (Path) 1 | 48.0 | Control (Path) 3 | 2.4 |
| Temporal Ctx | | Parietal Ctx | |
| Control (Path) 2 | 41.8 | Control (Path) 4 | 7.5 |
| Temporal Ctx | | Parietal Ctx | |

TABLE AHC
General_screening_panel_v1.4

| | Rel. Exp. (%) | | Rel. Exp. (%) |
|---|---|---|---|
| | Ag4412, | | Ag4412, |
| Tissue Name | Run 219923005 | Tissue Name | Run 219923005 |
| Adipose | 1.5 | Renal ca. TK-10 | 2.3 |
| Melanoma* | 0.3 | Bladder | 5.0 |
| Hs688(A).T | | | |
| Melanoma* | 0.0 | Gastric ca. (liver met.) | 0.0 |
| Hs688(B).T | | NCI-N87 | |
| Melanoma* M14 | 0.0 | Gastric ca. KATO III | 0.0 |
| Melanoma* LOXIMVI | 0.7 | Colon ca. SW-948 | 0.0 |
| Melanoma* SK-MEL-5 | 2.0 | Colon ca. SW480 | 0.4 |
| Squamous cell | 0.0 | Colon ca.* (SW480 met) | 0.0 |
| carcinoma SCC-4 | | SW620 | |
| Testis Pool | 6.7 | Colon ca. HT29 | 0.0 |
| Prostate ca.* (bone met) | 95.9 | Colon ca. HCT-116 | 3.9 |
| PC-3 | | | |
| Prostate Pool | 0.2 | Colon ca. CaCo-2 | 0.1 |
| Placenta | 0.0 | Colon cancer tissue | 0.9 |
| Uterus Pool | 0.5 | Colon ca. SW1116 | 0.5 |
| Ovarian ca. OVCAR-3 | 9.5 | Colon ca. Colo-205 | 0.0 |
| Ovarian ca. SK-OV-3 | 27.2 | Colon ca. SW-48 | 0.0 |
| Ovarian ca. OVCAR-4 | 0.3 | Colon Pool | 4.2 |
| Ovarian ca. OVCAR-5 | 1.5 | Small Intestine Pool | 6.4 |
| Ovarian ca. IGROV-1 | 2.0 | Stomach Pool | 3.3 |
| Ovarian ca. OVCAR-8 | 5.4 | Bone Marrow Pool | 0.7 |
| Ovary | 2.4 | Fetal Heart | 0.0 |
| Breast ca. MCF-7 | 12.6 | Heart Pool | 1.6 |
| Breast ca. MDA-MB- | 0.2 | Lymph Node Pool | 5.4 |
| 231 | | | |
| Breast ca. BT 549 | 4.5 | Fetal Skeletal Muscle | 0.0 |
| Breast ca. T47D | 7.9 | Skeletal Muscle Pool | 0.3 |
| Breast ca. MDA-N | 2.1 | Spleen Pool | 1.0 |
| Breast Pool | 6.0 | Thymus Pool | 6.1 |
| Trachea | 3.3 | CNS cancer (glio/astro) | 0.2 |
| | | U87-MG | |
| Lung | 0.8 | CNS cancer (glio/astro) U- | 0.2 |
| | | 118-MG | |
| Fetal Lung | 2.8 | CNS cancer (neuro; met) | 31.6 |
| | | SK-N-AS | |
| Lung ca. NCI-N417 | 1.1 | CNS cancer (astro) SF-539 | 1.7 |
| Lung ca. LX-1 | 0.3 | CNS cancer (astro) SNB- | 16.6 |
| | | 75 | |
| Lung ca. NCI-H146 | 9.2 | CNS cancer (glio) SNB-19 | 1.6 |
| Lung ca. SHP-77 | 27.7 | CNS cancer (glio) SF-295 | 1.5 |
| Lung ca. A549 | 6.3 | Brain (Amygdala) Pool | 6.7 |
| Lung ca. NCI-H526 | 0.0 | Brain (cerebellum) | 25.3 |
| Lung ca. NCI-H23 | 20.2 | Brain (fetal) | 100.0 |
| Lung ca. NCI-H460 | 9.5 | Brain (Hippocampus) Pool | 4.5 |
| Lung ca. HOP-62 | 0.9 | Cerebral Cortex Pool | 8.6 |
| Lung ca. NCI-H522 | 37.6 | Brain (Substantia nigra) | 6.0 |
| | | Pool | |
| Liver | 0.0 | Brain (Thalamus) Pool | 8.7 |
| Fetal Liver | 0.2 | Brain (whole) | 17.0 |
| Liver ca. HepG2 | 0.4 | Spinal Cord Pool | 6.8 |
| Kidney Pool | 6.8 | Adrenal Gland | 1.1 |
| Fetal Kidney | 0.7 | Pituitary gland Pool | 3.6 |
| Renal ca. 786-0 | 0.0 | Salivary Gland | 0.5 |
| Renal ca. A498 | 2.4 | Thyroid (female) | 2.4 |
| Renal ca. ACHN | 0.0 | Pancreatic ca. CAPAN2 | 1.8 |
| Renal ca. UO-31 | 1.3 | Pancreas Pool | 6.3 |

TABLE AHD
Oncology_cell_line_screening_panel_v3.2

| | Rel. | | Rel. |
|---|---|---|---|
| | Exp. (%) | | Exp. (%) |
| | Ag4412, | | Ag4412, |
| | Run | | Run |
| Tissue Name | 268695315 | Tissue Name | 268695315 |
| 94905_Daoy_Medulloblastoma/Cerebellum_{—} | 0.0 | 94954_Ca Ski_Cervical epidermoid | 0.0 |
| sscDNA | | carcinoma (metastasis)_sscDNA | |
| 94906_TE671_Medulloblastom/Cerebellum_{—} | 1.3 | 94955_ES-2_Ovarian clear cell | 0.0 |
| sscDNA | | carcinoma_sscDNA | |
| 94907_D283 | 12.4 | 94957_Ramos/6 h stim_Stimulated | 0.0 |
| Med_Medulloblastoma/Cerebellum_sscDNA | | with PMA/ionomycin 6 h_sscDNA | |
| 94908_PFSK-1_Primitive | 0.9 | 94958_Ramos/14 h stim_Stimulated | 0.0 |
| Neuroectodermal/Cerebellum_sscDNA | | with PMA/ionomycin 14 h_sscDNA | |
| 94909_XF-498_CNS_sscDNA | 2.9 | 94962_MEG-01_Chronic | 0.0 |
| | | myelogenous leukemia | |
| | | (megokaryoblast)_sscDNA | |
| 94910_SNB-78_CNS/glioma_sscDNA | 0.9 | 94963_Raji_Burkitt's | 0.0 |
| | | lymphoma_sscDNA | |
| 94911_SF- | 0.0 | 94964_Daudi_Burkitt's | 0.0 |
| 268_CNS/glioblastoma_sscDNA | | lymphoma_sscDNA | |
| 94912_T98G_Glioblastoma_sscDNA | 0.0 | 94965_U266_B-cell | 0.0 |
| | | plasmacytoma/myeloma_sscDNA | |
| 96776_SK-N-SH_Neuroblastoma | 4.2 | 94968_CA46_Burkitt's | 0.0 |
| (metastasis)_sscDNA | | lymphoma_sscDNA | |
| 94913_SF- | 0.0 | 94970_RL_non-Hodgkin's B-cell | 0.0 |
| 295_CNS/glioblastoma_sscDNA | | lymphoma_sscDNA | |
| 132565_NT2 pool_sscDNA | 11.2 | 94972_JM1_pre-B-cell | 0.0 |
| | | lymphoma/leukemia_sscDNA | 0.0 |
| 94914_Cerebellum_sscDNA | 24.5 | 94973_Jurkat_T cell | 0.0 |
| | | leukemia_sscDNA | |
| 96777_Cerebellum_sscDNA | 13.5 | 94974_TF- | 0.0 |
| | | 1_Erythroleukemia_sscDNA | |
| 94916_NCI-H292_Mucoepidermoid lung | 0.0 | 94975_HUT 78_T-cell | 0.0 |
| carcinoma_sscDNA | | lymphoma_sscDNA | |
| 94917_DMS-114_Small cell lung | 3.7 | 94977_U937_Histiocytic | 0.0 |
| cancer_sscDNA | | lymphoma_sscDNA | |
| 94918_DMS-79_Small cell lung | 100.0 | 94980_KU-812_Myelogenous | 0.0 |
| cancer/neuroendocrine_sscDNA | | leukemia_sscDNA | |
| 94919_NCI-H146_Small cell lung | 15.3 | 94981_769-P_Clear cell renal | 0.0 |
| cancer/neuroendocrine_sscDNA | | carcinoma_sscDNA | |
| 94920_NCI-H526_Small cell lung | 1.6 | 94983_Caki-2_Clear cell renal | 1.9 |
| cancer/neuroendocrine_sscDNA | | carcinoma_sscDNA | |
| 94921_NCI-N417_Small cell lung | 1.4 | 94984_SW 839_Clear cell renal | 1.5 |
| cancer/neuroendocrine_sscDNA | | carcinoma_sscDNA | |
| 94923_NCI-H82_Small cell lung | 1.4 | 94986_G401_Wilms' tumor_sscDNA | 4.0 |
| cancer/neuroendocrine_sscDNA | | | |
| 94924_NCI-H157_Squamous cell lung | 0.6 | 126768_293 cells_sscDNA | 0.0 |
| cancer (metastasis)_sscDNA | | | |
| 94925_NCI-H1155_Large cell lung | 19.5 | 94987_Hs766T_Pancreatic | 0.0 |
| cancer/neuroendocrine_sscDNA | | carcinoma (LN metastasis)_sscDNA | |
| 94926_NCI-H1299_Large cell lung | 5.0 | 94988_CAPAN-1_Pancreatic | 0.0 |
| cancer/neuroendocrine_sscDNA | | adenocarcinoma (liver | |
| | | metastasis)_sscDNA | |
| 94927_NCI-H727_Lung | 39.0 | 94989_SU86.86_Pancreatic | 4.5 |
| carcinoid_sscDNA | | carcinoma (liver | |
| | | metastasis)_sscDNA | |
| 94928_NCI-UMC-11_Lung | 18.3 | 94990_BxPC-3_Pancreatic | 0.0 |
| carcinoid_sscDNA | | adenocarcinoma_sscDNA | |
| 94929_LX-1_Small cell lung | 0.0 | 94991_HPAC_Pancreatic | 0.0 |
| cancer_sscDNA | | adenocarcinoma_sscDNA | |
| 94930_Colo-205_Colon cancer_sscDNA | 0.0 | 94992_MIA PaCa-2_Pancreatic | 0.0 |
| | | carcinoma_sscDNA | |
| 94931_KM12_Colon cancer_sscDNA | 13.0 | 94993_CFPAC-1_Pancreatic ductal | 11.4 |
| | | adenocarcinoma_sscDNA | |
| 94932_KM20L2_Colon cancer_sscDNA | 0.0 | 94994_PANC-1_Pancreatic | 0.0 |
| | | epithelioid ductal | |
| | | carcinoma_sscDNA | |
| 94933_NCI-H716_Colon | 13.4 | 94996_T24_Bladder carcinma | 0.0 |
| cancer_sscDNA | | (transitional cell)_sscDNA | |
| 94935_SW-48_Colon | 0.0 | 94997_5637_Bladder | 0.0 |
| adenocarcinoma_sscDNA | | carcinoma_sscDNA | |
| 94936_SW1116_Colon | 0.0 | 94998_HT-1197_Bladder | 0.0 |
| adenocarcinoma_sscDNA | | carcinoma_sscDNA | |
| 94937_LS 174T_Colon | 0.0 | 94999_UM-UC-3_Bladder carcinma | 0.0 |
| adenocarcinoma_sscDNA | | (transitional cell)_sscDNA | |
| 94938_SW-948_Colon | 0.0 | 95000_A204_Rhabdomyosarcoma_sscDNA | 2.4 |
| adenocarcinoma_sscDNA | | | |
| 94939_SW-480_Colon | 0.0 | 95001_HT- | 0.0 |
| adenocarcinoma_sscDNA | | 1080_Fibrosarcoma_sscDNA | |
| 94940_NCI-SNU-5_Gastric | 0.0 | 95002_MG-63_Osteosarcoma | 0.0 |
| carcinoma_sscDNA | | (bone)_sscDNA | |
| 112197_KATO III_Stomach_sscDNA | 0.0 | 95003_SK-LMS-1_Leiomyosarcoma | 4.6 |
| | | (vulva)_sscDNA | |
| 94943_NCI-SNU-16_Gastric | 0.7 | 95004_SJRH30_Rhabdomyosarcoma | 0.0 |
| carcinoma_sscDNA | | (met to bone marrow)_sscDNA | |
| 94944_NCI-SNU-1_Gastric | 0.0 | 95005_A431_Epidermoid | 0.0 |
| carcinoma_sscDNA | | carcinoma_sscDNA | |
| 94946_RF-1_Gastric | 0.0 | 95007_WM266- | 1.1 |
| adenocarcinoma_sscDNA | | 4_Melanoma_sscDNA | |
| 94947_RF-48_Gastric | 0.0 | 112195_DU 145_Prostate_sscDNA | 4.4 |
| adenocarcinoma_sscDNA | | | |
| 96778_MKN-45_Gastric | 4.4 | 95012_MDA-MB-468_Breast | 0.5 |
| carcinoma_sscDNA | | adenocarcinoma_sscDNA | |
| 94949_NCI-N87_Gastric | 0.7 | 112196_SSC-4_Tongue_sscDNA | 0.0 |
| carcinoma_sscDNA | | | |
| 94951_OVCAR-5_Ovarian | 0.0 | 112194_SSC-9_Tongue_sscDNA | 0.0 |
| carcinoma_sscDNA | | | |
| 94952_RL95-2_Uterine | 1.4 | 112191_SSC-15_Tongue_sscDNA | 0.0 |
| carcinoma_sscDNA | | | |
| 94953_HelaS3_Cervical | 0.0 | 95017_CAL 27_Squamous cell | 0.0 |
| adenocarcinoma_sscDNA | | carcinoma of tongue_sscDNA | |

TABLE AHE
Panel 4.1D

| | Rel. Exp. (%) | | Rel. Exp. (%) |
|---|---|---|---|
| | Ag4412, | | Ag4412, |
| Tissue Name | Run 190413471 | Tissue Name | Run 190413471 |
| Secondary Th1 act | 0.0 | HUVEC IL-1beta | 0.0 |
| Secondary Th2 act | 0.0 | HUVEC IFN gamma | 3.9 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 4.0 |
| Primary Th1 act | 0.0 | Lung Microvascular EC | 0.0 |
| | | TNF alpha + IL-1beta | |
| Primary Th2 act | 0.0 | Microvascular Dermal EC | 0.0 |
| | | none | |
| Primary Tr1 act | 0.0 | Microsvasular Dermal EC | 0.0 |
| | | TNF alpha + IL-1beta | |
| Primary Th1 rest | 0.0 | Bronchial epithelium | 11.4 |
| | | TNF alpha + IL-1beta | |
| Primary Th2 rest | 0.0 | Small airway epithelium none | 3.2 |
| Primary Tr1 rest | 0.0 | Small airway epithelium | 4.0 |
| | | TNF alpha + IL-1beta | |
| CD45RA CD4 | 0.0 | Coronery artery SMC rest | 0.0 |
| lymphocyte act | | | |
| CD45RO CD4 | 36.1 | Coronery artery SMC | 0.0 |
| lymphocyte act | | TNF alpha + IL-1beta | |
| CD8 lymphocyte act | 0.0 | Astrocytes rest | 3.8 |
| Secondary CD8 | 0.0 | Astrocytes TNF alpha + IL- | 4.9 |
| lymphocyte rest | | 1beta | |
| Secondary CD8 | 0.0 | KU-812 (Basophil) rest | 6.6 |
| lymphocyte act | | | |
| CD4 lymphocyte none | 0.0 | KU-812 (Basophil) | 5.1 |
| | | PMA/ionomycin | |
| 2ry Th1/Th2/Tr1_anti- | 0.0 | CCD1106 (Keratinocytes) | 25.9 |
| CD95 CH11 | | none | |
| LAK cells rest | 0.0 | CCD1106 (Keratinocytes) | 20.9 |
| | | TNF alpha + IL-1beta | |
| LAK cells IL-2 | 0.0 | Liver cirrhosis | 0.0 |
| LAK cells IL-2 + IL-12 | 0.0 | NCI-H292 none | 100.0 |
| LAK cells IL-2 + IFN | 0.0 | NCI-H292 IL-4 | 0.0 |
| gamma | | | |
| LAK cells IL-2 + IL-18 | 0.0 | NCI-H292 IL-9 | 0.0 |
| LAK cells | 0.0 | NCI-H292 IL-13 | 0.0 |
| PMA/ionomycin | | | |
| NK Cells IL-2 rest | 0.0 | NCI-H292 IFN gamma | 0.0 |
| Two Way MLR 3 day | 0.0 | HPAEC none | 0.0 |
| Two Way MLR 5 day | 4.2 | HPAEC TNF alpha + IL-1beta | 6.3 |
| Two Way MLR 7 day | 0.0 | Lung fibroblast none | 5.7 |
| PBMC rest | 0.0 | Lung fibroblast TNF alpha + | 0.0 |
| | | IL-1beta | |
| PBMC PWM | 0.0 | Lung fibroblast IL-4 | 0.0 |
| PBMC PHA-L | 0.0 | Lung fibroblast IL-9 | 0.0 |
| Ramos (B cell) none | 0.0 | Lung fibroblast IL-13 | 6.0 |
| Ramos (B cell) ionomycin | 0.0 | Lung fibroblast IFN gamma | 0.0 |
| B lymphocytes PWM | 0.0 | Dermal fibroblast CCD1070 | 0.0 |
| | | rest | |
| B lymphocytes CD40L | 0.0 | Dermal fibroblast CCD1070 | 0.0 |
| and IL-4 | | TNF alpha | |
| EOL-1 dbcAMP | 0.0 | Dermal fibroblast CCD1070 | 0.0 |
| | | IL-1beta | |
| EOL-1 dbcAMP | 0.0 | Dermal fibroblast IFN gamma | 0.0 |
| PMA/ionomycin | | | |
| Dendritic cells none | 0.0 | Dermal fibroblast IL-4 | 0.0 |
| Dendritic cells LPS | 0.0 | Dermal Fibroblasts rest | 0.0 |
| Dendritic cells anti-CD40 | 0.0 | Neutrophils TNFa + LPS | 0.0 |
| Monocytes rest | 0.0 | Neutrophils rest | 0.0 |
| Monocytes LPS | 0.0 | Colon | 3.2 |
| Macrophages rest | 0.0 | Lung | 20.3 |
| Macrophages LPS | 4.9 | Thymus | 0.0 |
| HUVEC none | 0.0 | Kidney | 21.3 |
| HUVEC starved | 4.5 | | |

TABLE AHF
general oncology screening panel_v_2.4

| | Rel. Exp. (%) | | Rel. Exp. (%) |
|---|---|---|---|
| | Ag4412, | | Ag4412, |
| Tissue Name | Run 264979086 | Tissue Name | Run 264979086 |
| Colon cancer 1 | 0.0 | Bladder cancer NAT 2 | 0.0 |
| Colon NAT 1 | 1.5 | Bladder cancer NAT 3 | 0.0 |
| Colon cancer 2 | 1.3 | Bladder cancer NAT 4 | 0.0 |
| Colon cancer NAT 2 | 0.0 | Adenocarcinoma of the | 6.5 |
| | | prostate 1 | |
| Colon cancer 3 | 1.8 | Adenocarcinoma of the | 0.6 |
| | | prostate 2 | |
| Colon cancer NAT 3 | 3.1 | Adenocarcinoma of the | 1.6 |
| | | prostate 3 | |
| Colon malignant | 5.4 | Adenocarcinoma of the | 1.6 |
| cancer 4 | | prostate 4 | |
| Colon normal adjacent | 0.0 | Prostate cancer NAT 5 | 0.5 |
| tissue 4 | | | |
| Lung cancer 1 | 9.0 | Adenocarcinoma of the | 0.0 |
| | | prostate 6 | |
| Lung NAT 1 | 0.0 | Adenocarcinoma of the | 1.4 |
| | | prostate 7 | |
| Lung cancer 2 | 22.4 | Adenocarcinoma of the | 0.0 |
| | | prostate 8 | |
| Lung NAT 2 | 2.0 | Adenocarcinoma of the | 1.9 |
| | | prostate 9 | |
| Squamous cell | 4.1 | Prostate cancer NAT 10 | 0.0 |
| carcinoma 3 | | | |
| Lung NAT 3 | 0.0 | Kidney cancer 1 | 12.6 |
| metastatic melanoma 1 | 4.7 | KidneyNAT 1 | 4.8 |
| Melanoma 2 | 0.0 | Kidney cancer 2 | 100.0 |
| Melanoma 3 | 0.0 | Kidney NAT 2 | 5.3 |
| metastatic melanoma 4 | 6.2 | Kidney cancer 3 | 2.3 |
| metastatic melanoma 5 | 5.7 | Kidney NAT 3 | 1.2 |
| Bladder cancer 1 | 0.0 | Kidney cancer 4 | 14.5 |
| Bladder cancer NAT 1 | 0.0 | Kidney NAT 4 | 2.4 |
| Bladder cancer 2 | 0.0 | | |

CNS_neurodegeneration_v1.0 Summary: Ag4412 This panel does not show differential expression of the CG110187-01 gene in Alzheimer's disease. However, this expression profile confirms the presence of this gene in the brain. Please see Panel 1.4 for discussion of use of this gene in the central nervous system.

General_screening_panel_v1.4 Summary: Ag4412 Highest expression of the CG110187-01 gene is seen in the fetal brain (CT=29.8). This gene is also expressed at moderate to low levels in all CNS regions examined, including the hippocampus, thalamus, substantia nigra, amygdala, cerebellum and cerebral cortex. The cadherins have been shown to be critical for CNS development, specifically for the guidance of axons, dendrites and/or growth cones in general. Therapeutic modulation of the levels of this protein, or possible signaling via this protein may be of utility in enhancing/directing compensatory synaptogenesis and fiber growth in the CNS in response to neuronal death (stroke, head trauma), axon lesion (spinal cord injury), or neurodegeneration (Alzheimer's, Parkinson's, Huntington's, vascular dementia or any neurodegenerative disease). Since protocadherins play an important role in synaptogenesis, this gene product may also be involved in depression, schizophrenia, which also involve synaptogeneisis. (Hilschmann N. Naturwissenschaften January 2001;88(1):2-12)

Moderate levels of expression are also seen in prostate, ovarian, lung and brain cancer cell lines. Thus, expression of this gene could be used to as a marker to detect the presence of these cancers. This gene encodes a protien that is homologous to cadherin which is involved in cellular adhesion. Dysregulation of cadherins has been observed in cancer, including renal cell carcinomas (Stassar, M J. Br J Cancer Nov. 2, 2000;85(9):1372-82). Therefore, therapeutic modulation of the expression or function of this gene may be effective in the treatment of prostate, ovarian, lung and brain cancers.

Oncology_cell_line_screening_panel_v3.2 Summary: Ag4412 Significant expression of the CG110817-01 gene is restricted to lung cancer cell lines and the cerebellum, with highest expression in a small cell lung cancer cell line (CT=32.4). This expression is in agreement with expression in Panel 1.4, where significant levels of expression are detected in the brain and cancer cell lines. Thus, expression of this gene could be used as a marker for lung cancer. Furthermore, therapeutic modulation of the expression or function of this gene may be effective in the treatment of lung cancer.

Panel 4.1D Summary: Ag4412 Significant expression of the CG110817-01 gene is restricted to untreated muco-epidermoid NCI-H292 cells (CT=34.9). Thus, the protein could be used to identify certain lung tumors similar to NCI-H292. This expression is in agreement with the previous panels, where significant levels of expression are detected in lung cancer cell lines. The encoded protein may also contribute to the normal function of the goblet cells within the lung. Therefore, designing therapeutics to this protein may be important for the treatment of emphysema and asthma as well as other lung diseases in which goblet cells or the mucus they produce have pathological consequences.

general oncology screening panel_v_{—}2.4 Summary: Ag4412 Highest expression of the CG110817-01 gene is seen in kidney cancer (CT=32.2). In addition, significant levels of expression are also seen in kidney cancer and lung cancer when compared to expression in the normal adjacent tissue. Thus, expression of this gene could be used as a marker of these cancers. Furthermore, therapeutic modulation of the expression or function of this gene may be effective in the treatment of lung cancer.

AI. CG110205-01 and CG110205-02: A Disintegrin-Like and Metalloprotease (Reprolysin Type) with Thrombospondin

Expression of gene CG110205-01 and CG110205-02 was assessed using the primer-probe sets Ag2430, Ag4413, Ag6546, Ag6645, Ag7012 and Ag7058, described in Tables AIA, AIB, AIC, AID, AIE and AIF. Results of the RTQ-PCR runs are shown in Tables AIG, AIH, AII, AIJ, AIK, AIL, AIM, AIN and AIO. Please note that CG110205-02 is the peptide containing reprolysin and thrombospondin type 1 domains of CG110205-01 and only recognized by probes Ag2430, and Ag4413.

TABLE AIA

Probe Name Ag2430

| | | | Start | SEQ ID |
|---|---|---|---|---|
| Primers | Sequences | Length | Position | No |
| Forward | 5′-cattggaaagaatggcaaga-3′ | 20 | 1209 | 270 |
| Probe | TET-5′-catgatcatgccatcttactaacagga-3′-TAMRA | 27 | 1231 | 271 |
| Reverse | 5′-tcacatggttcattcttccaa-3′ | 21 | 1272 | 272 |

TABLE AIB

Probe Name Ag4413

| | | | Start | SEQ ID |
|---|---|---|---|---|
| Primers | Sequences | Length | Position | No |
| Forward | 5′-ttggaagaatgaaccatgtga-3′ | 21 | 1272 | 273 |
| Probe | TET-5′-ccccatcagtggaatgtgctctaagt-3′-TAMRA | 26 | 1308 | 274 |
| Reverse | 5′-caagtcctgtgtcctcattgat-3′ | 22 | 1348 | 275 |

TABLE AIC

Probe Name Ag6546

| | | | Start | SEQ ID |
|---|---|---|---|---|
| Primers | Sequences | Length | Position | No. |
| Forward | 5′-ggatagcttggaagtatgcactt-3′ | 23 | 2633 | 276 |
| Probe | TET-5′-caaggtcatgaatggaactccaccag-3′-TAMRA | 26 | 2658 | 277 |
| Reverse | 5′-ctggcatccagcaggtatag-3′ | 20 | 2700 | 278 |

TABLE AID

Probe Name Ag6645

| | | | | |
|---|---|---|---|---|
| | | | Start | SEQ ID |
| Primers | Sequences | Length | Position | No |

| | | | | |
|---|---|---|---|---|
| Forward | 5′-ggatagcttggaagtatgcactt-3′ | 23 | 2633 | 279 |
| Probe | TET-5′-caaggtcatgaatggaactccaccag-3′-TAMRA | 26 | 2658 | 280 |
| Reverse | 5′-ctggcatccagcaggtatag-3′ | 20 | 2700 | 281 |

TABLE AIE

Probe Name Ag7012

| | | | Start | SEQ ID |
|---|---|---|---|---|
| Primers | Sequences | Length | Position | No |
| Forward | 5′-ctgccctccacaatgga-3′ | 17 | 2877 | 282 |
| Probe | TET-5′-ccttggaccctggtctcagtgttcca-3′-TAMRA | 26 | 2895 | 283 |
| Reverse | 5′-cagaggagttcacgcttcct-3′ | 20 | 2941 | 284 |

TABLE AIF

Probe Name Ag7058

| | | | Start | SEQ ID |
|---|---|---|---|---|
| Primers | Sequences | Length | Position | No |
| Forward | 5′-caagttgtctgctccatcagaa-3′ | 22 | 3380 | 285 |
| Probe | TET-5′-ccggtgctacgagcctgtaatacaaacttc-3′-TAMRA | 30 | 3406 | 286 |
| Reverse | 5′-gatcctctctcttttcaggagct-3′ | 23 | 3441 | 287 |

TABLE AIG
AI_comprehensive panel_v1.0

| | Rel. Exp. (%) | Rel. Exp. (%) | | Rel. Exp. (%) | Rel. Exp. (%) |
|---|---|---|---|---|---|
| | Ag4413, Run | Ag4413, Run | | Ag4413, Run | Ag4413, Run |
| Tissue Name | 248080021 | 251506632 | Tissue Name | 248080021 | 251506632 |
| 110967 COPD-F | 0.3 | 5.1 | 112427 Match | 0.8 | 1.4 |
| | | | Control Psoriasis-F | | |
| 110980 COPD-F | 0.2 | 0.0 | 112418 Psoriasis-M | 0.5 | 3.3 |
| 110968 COPD-M | 0.5 | 4.2 | 112723 Match | 2.3 | 22.8 |
| | | | Control Psoriasis-M | | |
| 110977 COPD-M | 0.2 | 0.5 | 112419 Psoriasis-M | 0.4 | 0.7 |
| 110989 | 0.5 | 33.2 | 112424 Match | 0.3 | 6.4 |
| Emphysema-F | | | Control Psoriasis-M | | |
| 110992 | 0.7 | 3.5 | 112420 Psoriasis-M | 1.2 | 7.3 |
| Emphysema-F | | | | | |
| 110993 | 0.4 | 7.9 | 112425 Match | 0.2 | 10.8 |
| Emphysema-F | | | Control Psoriasis-M | | |
| 110994 | 0.2 | 3.4 | 104689 (MF) OA | 7.5 | 17.1 |
| Emphysema-F | | | Bone-Backus | | |
| 110995 | 0.7 | 4.0 | 104690 (MF) Adj | 0.3 | 1.5 |
| Emphysema-F | | | “Normal” Bone- | | |
| | | | Backus | | |
| 110996 | 0.3 | 0.9 | 104691 (MF) OA | 0.2 | 3.6 |
| Emphysema-F | | | Synovium- | | |
| | | | Backus | | |
| 110997 Asthma-M | 0.3 | 3.0 | 104692 (BA) OA | 0.0 | 0.0 |
| | | | Cartilage-Backus | | |
| 111001 Asthma-F | 0.1 | 24.1 | 104694 (BA) OA | 23.7 | 3.9 |
| | | | Bone-Backus | | |
| 111002 Asthma-F | 0.3 | 22.4 | 104695 (BA) Adj | 2.6 | 6.3 |
| | | | “Normal” Bone- | | |
| | | | Backus | | |
| 111003 Atopic | 0.9 | 71.2 | 104696 (BA) OA | 1.0 | 16.6 |
| Asthma-F | | | Synovium- | | |
| | | | Backus | | |
| 111004 Atopic | 1.7 | 33.4 | 104700 (SS) OA | 2.2 | 2.3 |
| Asthma-F | | | Bone-Backus | | |
| 111005 Atopic | 1.6 | 38.2 | 104701 (SS) Adj | 5.9 | 4.1 |
| Asthma-F | | | “Normal” Bone- | | |
| | | | Backus | | |
| 111006 Atopic | 0.4 | 7.9 | 104702 (SS) OA | 1.1 | 60.3 |
| Asthma-F | | | Synovium- | | |
| | | | Backus | | |
| 111417 Allergy-M | 0.5 | 1.1 | 117093 OA | 0.2 | 3.2 |
| | | | Cartilage Rep7 | | |
| 112347 Allergy-M | 0.8 | 0.0 | 112672 OA | 0.1 | 0.6 |
| | | | Bone5 | | |
| 112349 Normal | 0.8 | 0.0 | 112673 OA | 0.1 | 0.0 |
| Lung-F | | | Synovium5 | | |
| 112357 Normal | 0.7 | 17.6 | 112674 OA | 0.1 | 0.6 |
| Lung-F | | | Synovial Fluid | | |
| | | | cells5 | | |
| 112354 Normal | 0.2 | 0.7 | 117100 OA | 0.1 | 0.5 |
| Lung-M | | | Cartilage Rep14 | | |
| 112374 Crohns-F | 1.4 | 6.3 | 112756 OA | 1.0 | 20.7 |
| | | | Bone9 | | |
| 112389 Match | 0.5 | 0.0 | 112757 OA | 0.0 | 0.0 |
| Control Crohns-F | | | Synovium9 | | |
| 112375 Crohns-F | 1.8 | 5.1 | 112758 OA | 0.5 | 12.9 |
| | | | Synovial Fluid | | |
| | | | Cells9 | | |
| 112732 Match | 0.0 | 0.0 | 117125 RA | 0.5 | 24.3 |
| Control Crohns-F | | | Cartilage Rep2 | | |
| 112725 Crohns-M | 0.4 | 0.0 | 113492 Bone2 | 0.0 | 1.4 |
| | | | RA | | |
| 112387 Match | 0.2 | 0.6 | 113493 | 0.0 | 0.0 |
| Control Crohns-M | | | Synovium2 RA | | |
| 112378 Crohns-M | 0.6 | 0.0 | 113494 Syn Fluid | 0.0 | 1.0 |
| | | | Cells RA | | |
| 112390 Match | 0.2 | 6.0 | 113499 | 0.0 | 0.0 |
| Control Crohns-M | | | Cartilage4 RA | | |
| 112726 Crohns-M | 1.9 | 100.0 | 113500 Bone4 | 0.0 | 0.0 |
| | | | RA | | |
| 112731 Match | 0.4 | 52.1 | 113501 | 0.0 | 0.7 |
| Control Crohns-M | | | Synovium4 RA | | |
| 112380 Ulcer | 0.6 | 29.3 | 113502 Syn Fluid | 0.0 | 1.5 |
| Col-F | | | Cells4 RA | | |
| 112734 Match | 100.0 | 0.6 | 113495 | 0.0 | 0.8 |
| Control Ulcer | | | Cartilage3 RA | | |
| Col-F | | | | | |
| 112384 Ulcer | 1.4 | 4.2 | 113496 Bone3 | 0.0 | 0.5 |
| Col-F | | | RA | | |
| 112737 Match | 1.2 | 52.1 | 113497 | 0.0 | 0.6 |
| Control Ulcer | | | Synovium3 RA | | |
| Col-F | | | | | |
| 112386 Ulcer | 0.1 | 0.7 | 113498 Syn Fluid | 0.0 | 0.0 |
| Col-F | | | Cells3 RA | | |
| 112738 Match | 0.1 | 0.0 | 117106 Normal | 0.1 | 1.3 |
| Control Ulcer | | | Cartilage Rep20 | | |
| Col-F | | | | | |
| 112381 Ulcer | 0.3 | 0.5 | 113663 Bone3 | 0.7 | 0.1 |
| Col-M | | | Normal | | |
| 112735 Match | 1.9 | 0.9 | 113664 | 0.1 | 0.0 |
| Control Ulcer | | | Synovium3 | | |
| Col-M | | | Normal | | |
| 112382 Ulcer | 0.4 | 0.0 | 113665 Syn Fluid | 0.4 | 0.0 |
| Col-M | | | Cells3 Normal | | |
| 112394 Match | 0.1 | 0.6 | 117107 Normal | 0.0 | 1.3 |
| Control Ulcer | | | Cartilage Rep22 | | |
| Col-M | | | | | |
| 112383 Ulcer | 1.8 | 18.7 | 113667 Bone4 | 0.2 | 0.9 |
| Col-M | | | Normal | | |
| 112736 Match | 1.6 | 0.0 | 113668 | 0.1 | 1.6 |
| Control Ulcer | | | Synovium4 | | |
| Col-M | | | Normal | | |
| 112423 Psoriasis-F | 1.0 | 11.2 | 113669 Syn Fluid | 0.2 | 1.9 |
| | | | Cells4 Normal | | |

TABLE AIH
CNS_neurodegeneration_v1.0

| | Rel. Exp. (%) | Rel. Exp. (%) | | Rel. Exp. (%) | Rel. Exp. (%) |
|---|---|---|---|---|---|
| | Ag2430, Run | Ag4413, Run | | Ag2430, Run | Ag4413, Run |
| Tissue Name | 208712834 | 224505949 | Tissue Name | 208712834 | 224505949 |
| AD 1 Hippo | 0.0 | 0.0 | Control (Path) | 2.2 | 3.0 |
| | | | 3 Temporal | | |
| | | | Ctx | | |
| AD 2 Hippo | 15.4 | 21.6 | Control (Path) | 1.1 | 4.0 |
| | | | 4 Temporal | | |
| | | | Ctx | | |
| AD 3 Hippo | 0.0 | 0.8 | AD 1 Occipital | 0.7 | 0.0 |
| | | | Ctx | | |
| AD 4 Hippo | 17.6 | 27.5 | AD 2 Occipital | 0.0 | 0.0 |
| | | | Ctx (Missing) | | |
| AD 5 Hippo | 13.9 | 22.5 | AD 3 Occipital | 0.0 | 0.0 |
| | | | Ctx | | |
| AD 6 Hippo | 43.2 | 0.0 | AD 4 Occipital | 52.5 | 94.0 |
| | | | Ctx | | |
| Control 2 | 62.9 | 85.9 | AD 5 Occipital | 27.7 | 25.5 |
| Hippo | | | Ctx | | |
| Control 4 | 0.0 | 1.6 | AD 6 Occipital | 24.0 | 27.7 |
| Hippo | | | Ctx | | |
| Control (Path) | 3.2 | 14.5 | Control 1 | 0.0 | 0.0 |
| 3 Hippo | | | Occipital Ctx | | |
| AD 1 | 0.9 | 0.7 | Control 2 | 52.1 | 66.0 |
| Temporal Ctx | | | Occipital Ctx | | |
| AD 2 | 21.9 | 37.6 | Control 3 | 3.1 | 11.1 |
| Temporal Ctx | | | Occipital Ctx | | |
| AD 3 | 0.0 | 0.0 | Control 4 | 0.0 | 0.0 |
| Temporal Ctx | | | Occipital Ctx | | |
| AD 4 | 57.0 | 97.9 | Control (Path) | 59.9 | 73.2 |
| Temporal Ctx | | | 1 Occipital Ctx | | |
| AD 5 Inf | 69.7 | 65.1 | Control (Path) | 6.6 | 10.1 |
| Temporal Ctx | | | 2 Occipital Ctx | | |
| AD 5 Sup | 38.2 | 26.2 | Control (Path) | 5.4 | 4.7 |
| Temporal Ctx | | | 3 Occipital Ctx | | |
| AD 6 Inf | 100.0 | 100.0 | Control (Path) | 0.0 | 0.0 |
| Temporal Ctx | | | 4 Occipital Ctx | | |
| AD 6 Sup | 36.6 | 37.6 | Control 1 | 0.0 | 0.0 |
| Temporal Ctx | | | Parietal Ctx | | |
| Control 1 | 0.0 | 0.8 | Control 2 | 37.6 | 38.4 |
| Temporal Ctx | | | Parietal Ctx | | |
| Control 2 | 36.6 | 45.7 | Control 3 | 6.3 | 6.6 |
| Temporal Ctx | | | Parietal Ctx | | |
| Control 3 | 9.3 | 12.5 | Control (Path) | 19.5 | 26.1 |
| Temporal Ctx | | | 1 Parietal Ctx | | |
| Control 3 | 0.0 | 1.4 | Control (Path) | 7.2 | 9.1 |
| Temporal Ctx | | | 2 Parietal Ctx | | |
| Control (Path) | 18.0 | 28.1 | Control (Path) | 0.0 | 4.6 |
| 1 Temporal | | | 3 Parietal Ctx | | |
| Ctx | | | | | |
| Control (Path) | 5.1 | 0.0 | Control (Path) | 0.0 | 0.9 |
| 2 Temporal | | | 4 Parietal Ctx | | |
| Ctx | | | | | |

TABLE AII
General_screening_panel_v1.4

| | Rel. Exp. (%) | | Rel. Exp. (%) |
|---|---|---|---|
| | Ag4413, | | Ag4413, |
| Tissue Name | Run 219923153 | Tissue Name | Run 219923153 |
| Adipose | 7.2 | Renal ca. TK-10 | 0.0 |
| Melanoma* | 0.0 | Bladder | 0.2 |
| Hs688(A).T | | | |
| Melanoma* | 0.0 | Gastric ca. (liver met.) | 0.0 |
| Hs688(B).T | | NCI-N87 | |
| Melanoma* M14 | 0.0 | Gastric ca. KATO III | 0.5 |
| Melanoma* LOXIMVI | 37.4 | Colon ca. SW-948 | 0.0 |
| Melanoma* SK-MEL-5 | 0.0 | Colon ca. SW480 | 0.0 |
| Squamous cell | 0.0 | Colon ca.* (SW480 met) | 49.0 |
| carcinoma SCC-4 | | SW620 | |
| Testis Pool | 1.8 | Colon ca. HT29 | 0.0 |
| Prostate ca.* (bone met) | 0.0 | Colon ca. HCT-116 | 0.0 |
| PC-3 | | | |
| Prostate Pool | 1.3 | Colon ca. CaCo-2 | 3.0 |
| Placenta | 19.5 | Colon cancer tissue | 2.6 |
| Uterus Pool | 0.9 | Colon ca. SW1116 | 0.0 |
| Ovarian ca. OVCAR-3 | 0.0 | Colon ca. Colo-205 | 0.0 |
| Ovarian ca. SK-OV-3 | 0.2 | Colon ca. SW-48 | 0.0 |
| Ovarian ca. OVCAR-4 | 0.0 | Colon Pool | 11.2 |
| Ovarian ca. OVCAR-5 | 0.0 | Small Intestine Pool | 1.7 |
| Ovarian ca. IGROV-1 | 0.1 | Stomach Pool | 6.3 |
| Ovarian ca. OVCAR-8 | 3.0 | Bone Marrow Pool | 2.7 |
| Ovary | 19.2 | Fetal Heart | 0.3 |
| Breast ca. MCF-7 | 0.0 | Heart Pool | 1.5 |
| Breast ca. MDA-MB- | 0.0 | Lymph Node Pool | 20.3 |
| 231 | | | |
| Breast ca. BT 549 | 1.1 | Fetal Skeletal Muscle | 3.0 |
| Breast ca. T47D | 0.0 | Skeletal Muscle Pool | 0.0 |
| Breast ca. MDA-N | 0.0 | Spleen Pool | 0.0 |
| Breast Pool | 11.7 | Thymus Pool | 8.4 |
| Trachea | 0.9 | CNS cancer (glio/astro) | 39.0 |
| | | U87-MG | |
| Lung | 0.6 | CNS cancer (glio/astro) U- | 1.8 |
| | | 118-MG | |
| Fetal Lung | 0.6 | CNS cancer (neuro; met) | 0.2 |
| | | SK-N-AS | |
| Lung ca. NCI-N417 | 0.0 | CNS cancer (astro) SF-539 | 0.0 |
| Lung ca. LX-1 | 9.7 | CNS cancer (astro) SNB- | 0.7 |
| | | 75 | |
| Lung ca. NCI-H146 | 3.1 | CNS cancer (glio) SNB-19 | 0.2 |
| Lung ca. SHP-77 | 0.0 | CNS cancer (glio) SF-295 | 1.3 |
| Lung ca. A549 | 0.0 | Brain (Amygdala) Pool | 4.0 |
| Lung ca. NCI-H526 | 5.4 | Brain (cerebellum) | 100.0 |
| Lung ca. NCI-H23 | 0.9 | Brain (fetal) | 2.7 |
| Lung ca. NCI-H460 | 0.0 | Brain (Hippocampus) Pool | 2.0 |
| Lung ca. HOP-62 | 0.4 | Cerebral Cortex Pool | 4.3 |
| Lung ca. NCI-H522 | 0.0 | Brain (Substantia nigra) | 5.5 |
| | | Pool | |
| Liver | 0.0 | Brain (Thalamus) Pool | 8.7 |
| Fetal Liver | 0.7 | Brain (whole) | 11.8 |
| Liver ca. HepG2 | 0.2 | Spinal Cord Pool | 8.0 |
| Kidney Pool | 2.1 | Adrenal Gland | 0.0 |
| Fetal Kidney | 17.1 | Pituitary gland Pool | 3.2 |
| Renal ca. 786-0 | 0.0 | Salivary Gland | 6.8 |
| Renal ca. A498 | 0.0 | Thyroid (female) | 0.3 |
| Renal ca. ACHN | 3.1 | Pancreatic ca. CAPAN2 | 0.0 |
| Renal ca. UO-31 | 30.1 | Pancreas Pool | 8.2 |

TABLE AIJ
Oncology_cell_line_screening_panel_v3.2

| | Rel. Exp. (%) | | Rel. Exp. (%) |
|---|---|---|---|
| | Ag2430, Run | | Ag2430, Run |
| Tissue Name | 258381230 | Tissue Name | 258381230 |
| 94905_Daoy_Medulloblastoma/Cerebellum_{—} | 0.0 | 94954_Ca Ski_Cervical | 0.0 |
| sscDNA | | epidermoid carcinoma | |
| | | (metastasis)_sscDNA | |
| 94906_TE671_Medulloblastom/Cerebellum_{—} | 0.0 | 94955_ES-2_Ovarian clear cell | 0.0 |
| sscDNA | | carcinoma_sscDNA | |
| 94907_D283 | 0.0 | 94957_Ramos/6 h stim_{—} | 0.0 |
| Med_Medulloblastoma/Cerebellum_{—} | | Stimulated with PMA/ionomycin | |
| sscDNA | | 6 h_sscDNA | |
| 94908_PFSK-1_Primitive | 0.0 | 94958_Ramos/14 h stim_{—} | 0.0 |
| Neuroectodermal/Cerebellum_sscDNA | | Stimulated with PMA/ionomycin | |
| | | 14 h_sscDNA | |
| 94909_XF-498_CNS_sscDNA | 0.0 | 94962_MEG-01_Chronic | 0.0 |
| | | myelogenous leukemia | |
| | | (megokaryoblast)_sscDNA | |
| 94910_SNB- | 0.0 | 94963_Raji_Burkitt's | 0.0 |
| 78_CNS/glioma_sscDNA | | lymphoma_sscDNA | |
| 94911_SF- | 0.0 | 94964_Daudi_Burkitt's | 0.0 |
| 268_CNS/glioblastoma_sscDNA | | lymphoma_sscDNA | |
| 94912_T98G_Glioblastoma_sscDNA | 0.0 | 94965_U266_B-cell | 0.0 |
| | | plasmacytoma/myeloma_sscDNA | |
| 96776_SK-N-SH_Neuroblastoma | 0.0 | 94968_CA46_Burkitt's | 0.0 |
| (metastasis)_sscDNA | | lymphoma_sscDNA | |
| 94913_SF- | 0.0 | 94970_RL_non-Hodgkin's B-cell | 0.0 |
| 295_CNS/glioblastoma_sscDNA | | lymphoma_sscDNA | |
| 132565_NT2 pool_sscDNA | 0.1 | 94972_JM1_pre-B-cell | 0.0 |
| | | lymphoma/leukemia_sscDNA | |
| 94914_Cerebellum_sscDNA | 57.8 | 94973_Jurkat_T cell | 0.0 |
| | | leukemia_sscDNA | |
| 96777_Cerebellum_sscDNA | 25.5 | 94974_TF- | 0.0 |
| | | 1_Erythroleukemia_sscDNA | |
| 94916_NCI- | 0.0 | 94975_HUT 78_T-cell | 0.0 |
| H292_Mucoepidermoid lung | | lymphoma_sscDNA | |
| carcinoma_sscDNA | | | |
| 94917_DMS-114_Small cell lung | 17.9 | 94977_U937_Histiocytic | 0.0 |
| cancer_sscDNA | | lymphoma_sscDNA | |
| 94918_DMS-79_Small cell lung | 100.0 | 94980_KU-812_Myelogenous | 0.0 |
| cancer/neuroendocrine_sscDNA | | leukemia_sscDNA | |
| 94919_NCI-H146_Small cell lung | 2.9 | 94981_769-P_Clear cell renal | 0.0 |
| cancer/neuroendocrine_sscDNA | | carcinoma_sscDNA | |
| 94920_NCI-H526_Small cell lung | 10.9 | 94983_Caki-2_Clear cell renal | 0.0 |
| cancer/neuroendocrine_sscDNA | | carcinoma_sscDNA | |
| 94921_NCI-N417_Small cell lung | 0.0 | 94984_SW 839_Clear cell renal | 0.0 |
| cancer/neuroendocrine_sscDNA | | carcinoma_sscDNA | |
| 94923_NCI-H82_Small cell lung | 0.0 | 94986_G401_Wilms' | 0.0 |
| cancer/neuroendocrine_sscDNA | | tumor_sscDNA | |
| 94924_NCI-H157_Squamous cell | 0.0 | 126768_293 cells_sscDNA | 0.0 |
| lung cancer (metastasis)_sscDNA | | | |
| 94925_NCI-H1155_Large cell | 0.0 | 94987_Hs766T_Pancreatic | 0.0 |
| lung | | carcinoma (LN | |
| cancer/neuroendocrine_sscDNA | | metastasis)_sscDNA | |
| 94926_NCI-H1299_Large cell | 0.0 | 94988_CAPAN-1_Pancreatic | 0.0 |
| lung | | adenocarcinoma (liver | |
| cancer/neuroendocrine_sscDNA | | metastasis)_sscDNA | |
| 94927_NCI-H727_Lung | 0.3 | 94989_SU86.86_Pancreatic | 0.0 |
| carcinoid_sscDNA | | carcinoma (liver | |
| | | metastasis)_sscDNA | |
| 94928_NCI-UMC-11_Lung | 0.0 | 94990_BxPC-3_Pancreatic | 0.0 |
| carcinoid_sscDNA | | adenocarcinoma_sscDNA | |
| 94929_LX-1_Small cell lung | 3.8 | 94991_HPAC_Pancreatic | 0.0 |
| cancer_sscDNA | | adenocarcinoma_sscDNA | |
| 94930_Colo-205_Colon | 0.0 | 94992_MIA PaCa-2_Pancreatic | 0.0 |
| cancer_sscDNA | | carcinoma_sscDNA | |
| 94931_KM12_Colon | 0.0 | 94993_CFPAC-1_Pancreatic | 0.0 |
| cancer_sscDNA | | ductal adenocarcinoma_sscDNA | |
| 94932_KM20L2_Colon | 0.0 | 94994_PANC-1_Pancreatic | 0.0 |
| cancer_sscDNA | | epithelioid ductal | |
| | | carcinoma_sscDNA | |
| 94933_NCI-H716_Colon | 0.0 | 94996_T24_Bladder carcinma | 0.0 |
| cancer_sscDNA | | (transitional cell)_sscDNA | |
| 94935_SW-48_Colon | 0.0 | 94997_5637_Bladder | 0.0 |
| adenocarcinoma_sscDNA | | carcinoma_sscDNA | |
| 94936_SW1116_Colon | 0.0 | 94998_HT-1197_Bladder | 0.0 |
| adenocarcinoma_sscDNA | | carcinoma_sscDNA | |
| 94937_LS 174T_Colon | 0.0 | 94999_UM-UC-3_Bladder | 0.0 |
| adenocarcinoma_sscDNA | | carcinma (transitional | |
| | | cell)_sscDNA | |
| 94938_SW-948_Colon | 0.0 | 95000_A204_Rhabdomyosarcoma_{—} | 0.4 |
| adenocarcinoma_sscDNA | | sscDNA | |
| 94939_SW-480_Colon | 0.0 | 95001_HT- | 0.2 |
| adenocarcinoma_sscDNA | | 1080_Fibrosarcoma_sscDNA | |
| 94940_NCI-SNU-5_Gastric | 0.0 | 95002_MG-63_Osteosarcoma | 0.0 |
| carcinoma_sscDNA | | (bone)_sscDNA | |
| 112197_KATO | 0.0 | 95003_SK-LMS- | 0.0 |
| III_Stomach_sscDNA | | 1_Leiomyosarcoma | |
| | | (vulva)_sscDNA | |
| 94943_NCI-SNU-16_Gastric | 0.0 | 95004_SJRH30_Rhabdomyosarcoma | 0.0 |
| carcinoma_sscDNA | | (met to bone | |
| | | marrow)_sscDNA | |
| 94944_NCI-SNU-1_Gastric | 0.0 | 95005_A431_Epidermoid | 0.0 |
| carcinoma_sscDNA | | carcinoma_sscDNA | |
| 94946_RF-1_Gastric | 0.0 | 95007_WM266- | 0.0 |
| adenocarcinoma_sscDNA | | 4_Melanoma_sscDNA | |
| 94947_RF-48_Gastric | 0.0 | 112195_DU | 0.0 |
| adenocarcinoma_sscDNA | | 145_Prostate_sscDNA | |
| 96778_MKN-45_Gastric | 0.0 | 95012_MDA-MB-468_Breast | 0.0 |
| carcinoma_sscDNA | | adenocarcinoma_sscDNA | |
| 94949_NCI-N87_Gastric | 0.0 | 112196_SSC-4_Tongue_sscDNA | 0.0 |
| carcinoma_sscDNA | | | |
| 94951_OVCAR-5_Ovarian | 0.0 | 112194_SSC-9_Tongue_sscDNA | 0.0 |
| carcinoma_sscDNA | | | |
| 94952_RL95-2_Uterine | 0.0 | 112191_SSC-15_Tongue_sscDNA | 0.0 |
| carcinoma_sscDNA | | | |
| 94953_HelaS3_Cervical | 0.0 | 95017_CAL 27_Squamous cell | 0.0 |
| adenocarcinoma_sscDNA | | carcinoma of tongue_sscDNA | |

TABLE AIK
Panel 1.3D

| | Rel. Exp. (%) | | Rel. Exp. (%) |
|---|---|---|---|
| | Ag2430, | | Ag2430, |
| Tissue Name | Run 159505456 | Tissue Name | Run 159505456 |
| Liver adenocarcinoma | 0.0 | Kidney (fetal) | 9.3 |
| Pancreas | 0.0 | Renal ca. 786-0 | 0.0 |
| Pancreatic ca. CAPAN 2 | 0.0 | Renal ca. A498 | 0.5 |
| Adrenal gland | 1.1 | Renal ca. RXF 393 | 0.0 |
| Thyroid | 0.2 | Renal ca. ACHN | 3.8 |
| Salivary gland | 15.0 | Renal ca. UO-31 | 13.9 |
| Pituitary gland | 13.2 | Renal ca. TK-10 | 0.0 |
| Brain (fetal) | 0.3 | Liver | 0.0 |
| Brain (whole) | 40.9 | Liver (fetal) | 0.0 |
| Brain (amygdala) | 9.7 | Liver ca. (hepatoblast) | 0.0 |
| | | HepG2 | |
| Brain (cerebellum) | 100.0 | Lung | 0.0 |
| Brain (hippocampus) | 37.9 | Lung (fetal) | 2.6 |
| Brain (substantia nigra) | 8.0 | Lung ca. (small cell) | 9.9 |
| | | LX-1 | |
| Brain (thalamus) | 12.2 | Lung ca. (small cell) | 29.5 |
| | | NCI-H69 | |
| Cerebral Cortex | 4.1 | Lung ca. (s. cell var.) | 0.0 |
| | | SHP-77 | |
| Spinal cord | 6.4 | Lung ca. (large | 0.0 |
| | | cell)NCI-H460 | |
| glio/astro U87-MG | 59.9 | Lung ca. (non-sm. cell) | 0.0 |
| | | A549 | |
| glio/astro U-118-MG | 6.7 | Lung ca. (non-s. cell) | 2.0 |
| | | NCI-H23 | |
| astrocytoma SW1783 | 0.0 | Lung ca. (non-s. cell) | 0.0 |
| | | HOP-62 | |
| neuro*; met SK-N-AS | 0.0 | Lung ca. (non-s. cl) NCI- | 0.0 |
| | | H522 | |
| astrocytoma SF-539 | 0.0 | Lung ca. (squam.) SW | 0.0 |
| | | 900 | |
| astrocytoma SNB-75 | 0.6 | Lung ca. (squam.) NCI- | 0.8 |
| | | H596 | |
| glioma SNB-19 | 0.3 | Mammary gland | 30.8 |
| glioma U251 | 17.8 | Breast ca.* (pl. ef) MCF-7 | 0.0 |
| glioma SF-295 | 0.5 | Breast ca.* (pl. ef) | 0.0 |
| | | MDA-MB-231 | |
| Heart (fetal) | 0.0 | Breast ca.* (pl. ef) T47D | 0.0 |
| Heart | 0.0 | Breast ca. BT-549 | 31.9 |
| Skeletal muscle (fetal) | 13.5 | Breast ca. MDA-N | 0.3 |
| Skeletal muscle | 0.0 | Ovary | 32.8 |
| Bone marrow | 0.0 | Ovarian ca. OVCAR-3 | 0.0 |
| Thymus | 3.1 | Ovarian ca. OVCAR-4 | 0.0 |
| Spleen | 0.0 | Ovarian ca. OVCAR-5 | 0.0 |
| Lymph node | 0.6 | Ovarian ca. OVCAR-8 | 0.0 |
| Colorectal | 0.9 | Ovarian ca. IGROV-1 | 0.2 |
| Stomach | 0.0 | Ovarian ca.* (ascites) | 0.0 |
| | | SK-OV-3 | |
| Small intestine | 0.3 | Uterus | 3.0 |
| Colon ca. SW480 | 0.0 | Placenta | 47.0 |
| Colon ca.* | 75.3 | Prostate | 1.4 |
| SW620(SW480 met) | | | |
| Colon ca. HT29 | 0.0 | Prostate ca.* (bone | 0.0 |
| | | met)PC-3 | |
| Colon ca. HCT-116 | 0.0 | Testis | 1.2 |
| Colon ca. CaCo-2 | 2.8 | Melanoma Hs688(A).T | 0.0 |
| Colon ca. | 5.1 | Melanoma* (met) | 0.0 |
| tissue(ODO3866) | | Hs688(B).T | |
| Colon ca. HCC-2998 | 0.0 | Melanoma UACC-62 | 0.0 |
| Gastric ca.* (liver met) | 0.0 | Melanoma M14 | 0.0 |
| NCI-N87 | | | |
| Bladder | 1.7 | Melanoma LOX IMVI | 45.4 |
| Trachea | 2.2 | Melanoma* (met) SK- | 0.0 |
| | | MEL-5 | |
| Kidney | 0.2 | Adipose | 18.6 |

TABLE AIL
Panel 2D

| | Rel. Exp. (%) Ag2430, | | Rel. Exp. (%) Ag2430, |
|---|---|---|---|
| Tissue Name | Run 159505825 | Tissue Name | Run 159505825 |
| Normal Colon | 15.4 | Kidney Margin 8120608 | 0.6 |
| CC Well to Mod Diff | 12.9 | Kidney Cancer 8120613 | 5.1 |
| (ODO3866) | | | |
| CC Margin (ODO3866) | 0.0 | Kidney Margin 8120614 | 6.1 |
| CC Gr.2 rectosigmoid | 0.3 | Kidney Cancer 9010320 | 9.7 |
| (ODO3868) | | | |
| CC Margin (ODO3868) | 3.0 | Kidney Margin 9010321 | 2.7 |
| CC Mod Diff (ODO3920) | 5.3 | Normal Uterus | 23.8 |
| CC Margin (ODO3920) | 2.3 | Uterus Cancer 064011 | 25.5 |
| CC Gr.2 ascend colon | 12.7 | Normal Thyroid | 2.9 |
| (ODO3921) | | | |
| CC Margin (ODO3921) | 4.6 | Thyroid Cancer 064010 | 23.8 |
| CC from Partial Hepatectomy | 0.0 | Thyroid Cancer A302152 | 14.3 |
| (ODO4309) Mets | | | |
| Liver Margin (ODO4309) | 0.0 | Thyroid Margin A302153 | 5.6 |
| Colon mets to lung | 0.0 | Normal Breast | 58.6 |
| (OD04451-01) | | | |
| Lung Margin (OD04451-02) | 0.0 | Breast Cancer (OD04566) | 2.3 |
| Normal Prostate 6546-1 | 1.0 | Breast Cancer (OD04590- | 30.6 |
| | | 01) | |
| Prostate Cancer (OD04410) | 1.2 | Breast Cancer Mets | 17.0 |
| | | (OD04590-03) | |
| Prostate Margin (OD04410) | 47.0 | Breast Cancer Metastasis | 6.6 |
| | | (OD04655-05) | |
| Prostate Cancer (OD04720- | 7.9 | Breast Cancer 064006 | 7.1 |
| 01) | | | |
| Prostate Margin (OD04720- | 0.0 | Breast Cancer 1024 | 59.0 |
| 02) | | | |
| Normal Lung 061010 | 11.4 | Breast Cancer 9100266 | 33.0 |
| Lung Met to Muscle | 1.2 | Breast Margin 9100265 | 26.6 |
| (ODO4286) | | | |
| Muscle Margin (ODO4286) | 6.8 | Breast Cancer A209073 | 43.8 |
| Lung Malignant Cancer | 10.9 | Breast Margin A209073 | 68.3 |
| (OD03126) | | | |
| Lung Margin (OD03126) | 2.5 | Normal Liver | 0.0 |
| Lung Cancer (OD04404) | 2.3 | Liver Cancer 064003 | 0.8 |
| Lung Margin (OD04404) | 24.7 | Liver Cancer 1025 | 0.0 |
| Lung Cancer (OD04565) | 6.9 | Liver Cancer 1026 | 3.0 |
| Lung Margin (OD04565) | 1.2 | Liver Cancer 6004-T | 0.0 |
| Lung Cancer (OD04237-01) | 100.0 | Liver Tissue 6004-N | 2.9 |
| Lung Margin (OD04237-02) | 9.8 | Liver Cancer 6005-T | 0.0 |
| Ocular Mel Met to Liver | 0.0 | Liver Tissue 6005-N | 0.0 |
| (ODO4310) | | | |
| Liver Margin (ODO4310) | 0.0 | Normal Bladder | 4.8 |
| Melanoma Mets to Lung | 1.5 | Bladder Cancer 1023 | 2.5 |
| (OD04321) | | | |
| Lung Margin (OD04321) | 0.0 | Bladder Cancer A302173 | 16.3 |
| Normal Kidney | 24.7 | Bladder Cancer | 6.1 |
| | | (OD04718-01) | |
| Kidney Ca, Nuclear grade 2 | 6.4 | Bladder Normal Adjacent | 53.2 |
| (OD04338) | | (OD04718-03) | |
| Kidney Margin (OD04338) | 10.5 | Normal Ovary | 18.2 |
| Kidney Ca Nuclear grade 1/2 | 5.5 | Ovarian Cancer 064008 | 30.1 |
| (OD04339) | | | |
| Kidney Margin (OD04339) | 3.8 | Ovarian Cancer | 2.7 |
| | | (OD04768-07) | |
| Kidney Ca, Clear cell type | 14.1 | Ovary Margin | 6.6 |
| (OD04340) | | (OD04768-08) | |
| Kidney Margin (OD04340) | 3.1 | Normal Stomach | 0.8 |
| Kidney Ca, Nuclear grade 3 | 2.0 | Gastric Cancer 9060358 | 3.9 |
| (OD04348) | | | |
| Kidney Margin (OD04348) | 5.0 | Stomach Margin 9060359 | 0.0 |
| Kidney Cancer (OD04622-01) | 1.0 | Gastric Cancer 9060395 | 8.0 |
| Kidney Margin (OD04622- | 0.0 | Stomach Margin 9060394 | 1.8 |
| 03) | | | |
| Kidney Cancer (OD04450-01) | 6.5 | Gastric Cancer 9060397 | 12.2 |
| Kidney Margin (OD04450- | 11.3 | Stomach Margin 9060396 | 0.0 |
| 03) | | | |
| Kidney Cancer 8120607 | 2.6 | Gastric Cancer 064005 | 10.7 |

TABLE AIM
Panel 4.1D

| | Rel. | Rel. | Rel. | | Rel. | Rel. | Rel. |
|---|---|---|---|---|---|---|---|
| | Exp. (%) | Exp. (%) | Exp. (%) | | Exp. (%) | Exp. (%) | Exp. (%) |
| | Ag4413, | Ag4413, | Ag7012, | | Ag4413, | Ag4413, | Ag7012, |
| | Run | Run | Run | | Run | Run | Run |
| Tissue Name | 190281896 | 249495488 | 279065633 | Tissue Name | 190281896 | 249495488 | 279065633 |
| Secondary Th1 act | 0.0 | 0.0 | 0.0 | HUVEC IL- | 16.6 | 12.1 | 24.7 |
| | | | | 1beta | | | |
| Secondary Th2 act | 0.4 | 0.0 | 0.0 | HUVEC IFN | 52.9 | 53.2 | 57.8 |
| | | | | gamma | | | |
| Secondary Tr1 act | 0.6 | 0.0 | 0.0 | HUVEC TNF | 5.0 | 0.0 | 4.2 |
| | | | | alpha + IFN | | | |
| | | | | gamma | | | |
| Secondary Th1 | 1.1 | 0.0 | 0.0 | HUVEC TNF | 6.4 | 0.0 | 0.9 |
| rest | | | | alpha + IL4 | | | |
| Secondary Th2 | 0.0 | 0.0 | 0.0 | HUVEC IL-11 | 49.3 | 54.0 | 79.0 |
| rest | | | | | | | |
| Secondary Tr1 | 0.0 | 0.0 | 0.0 | Lung | 33.0 | 25.0 | 66.0 |
| rest | | | | Microvascular | | | |
| | | | | EC none | | | |
| Primary Th1 act | 0.0 | 0.0 | 0.0 | Lung | 42.9 | 8.7 | 17.2 |
| | | | | Microvascular | | | |
| | | | | EC TNF alpha + | | | |
| | | | | IL-1beta | | | |
| Primary Th2 act | 0.0 | 0.0 | 0.0 | Microvascular | 15.7 | 0.4 | 4.8 |
| | | | | Dermal EC none | | | |
| Primary Tr1 act | 0.0 | 0.0 | 0.0 | Microsvasular | 5.5 | 3.0 | 4.2 |
| | | | | Dermal EC | | | |
| | | | | TNF alpha + IL- | | | |
| | | | | 1beta | | | |
| Primary Th1 rest | 0.0 | 0.0 | 0.0 | Bronchial | 0.0 | 0.0 | 0.0 |
| | | | | epithelium | | | |
| | | | | TNF alpha + | | | |
| | | | | IL1beta | | | |
| Primary Th2 rest | 0.0 | 0.0 | 0.0 | Small airway | 0.0 | 0.0 | 0.0 |
| | | | | epithelium none | | | |
| Primary Tr1 rest | 0.0 | 0.0 | 0.0 | Small airway | 0.0 | 0.0 | 0.0 |
| | | | | epithelium | | | |
| | | | | TNF alpha + IL- | | | |
| | | | | 1beta | | | |
| CD45RA CD4 | 0.0 | 0.0 | 0.0 | Coronery artery | 3.6 | 2.8 | 0.5 |
| lymphocyte act | | | | SMC rest | | | |
| CD45RO CD4 | 0.0 | 0.0 | 0.0 | Coronery artery | 1.9 | 2.8 | 1.0 |
| lymphocyte act | | | | SMC TNF alpha + | | | |
| | | | | IL-1beta | | | |
| CD8 lymphocyte | 0.0 | 0.0 | 0.0 | Astrocytes rest | 0.0 | 0.9 | 0.0 |
| act | | | | | | | |
| Secondary CD8 | 0.0 | 0.0 | 0.0 | Astrocytes | 0.0 | 0.0 | 0.0 |
| lymphocyte rest | | | | TNF alpha + IL- | | | |
| | | | | 1beta | | | |
| Secondary CD8 | 0.0 | 0.0 | 0.0 | KU-812 | 5.8 | 0.0 | 0.0 |
| lymphocyte act | | | | (Basophil) rest | | | |
| CD4 lymphocyte | 0.0 | 0.0 | 0.0 | KU-812 | 0.9 | 0.0 | 0.0 |
| none | | | | (Basophil) | | | |
| | | | | PMA/ionomycin | | | |
| 2ry | 0.0 | 0.0 | 0.0 | CCD1106 | 0.0 | 0.0 | 0.0 |
| Th1/Th2/Tr1_anti- | | | | (Keratinocytes) | | | |
| CD95 CH11 | | | | none | | | |
| LAK cells rest | 0.0 | 0.0 | 0.0 | CCD1106 | 0.0 | 0.0 | 0.0 |
| | | | | (Keratinocytes) | | | |
| | | | | TNF alpha + IL- | | | |
| | | | | 1beta | | | |
| LAK cells IL-2 | 0.0 | 0.0 | 0.0 | Liver cirrhosis | 0.4 | 0.0 | 0.0 |
| LAX cells IL- | 0.0 | 0.0 | 0.0 | NCI-H292 none | 0.0 | 0.0 | 0.0 |
| 2 + IL-12 | | | | | | | |
| LAK cells IL- | 0.0 | 0.0 | 0.0 | NCI-H292 IL-4 | 0.0 | 0.0 | 0.0 |
| 2 + IFN gamma | | | | | | | |
| LAK cells IL-2 + | 0.0 | 0.0 | 0.0 | NCI-H292 IL-9 | 0.0 | 0.0 | 0.0 |
| IL-18 | | | | | | | |
| LAK cells | 0.0 | 0.0 | 0.0 | NCI-H292 IL-13 | 0.0 | 0.0 | 0.0 |
| PMA/ionomycin | | | | | | | |
| NK Cells IL-2 rest | 0.0 | 0.0 | 0.0 | NCI-H292 IFN | 0.0 | 0.0 | 0.0 |
| | | | | gamma | | | |
| Two Way MLR 3 | 0.0 | 0.0 | 0.0 | HPAEC none | 71.2 | 25.7 | 35.1 |
| day | | | | | | | |
| Two Way MLR 5 | 2.2 | 0.0 | 0.0 | HPAEC TNF | 100.0 | 100.0 | 100.0 |
| day | | | | alpha + IL-1 | | | |
| | | | | beta | | | |
| Two Way MLR 7 | 0.0 | 0.0 | 0.0 | Lung fibroblast | 0.0 | 0.0 | 0.0 |
| day | | | | none | | | |
| PBMC rest | 0.0 | 0.0 | 0.0 | Lung fibroblast | 0.3 | 0.0 | 0.0 |
| | | | | TNF alpha + IL- | | | |
| | | | | 1beta | | | |
| PBMC PWM | 0.0 | 0.0 | 0.0 | Lung fibroblast | 0.3 | 0.0 | 0.0 |
| | | | | IL-4 | | | |
| PBMC PHA-L | 0.0 | 0.0 | 0.0 | Lung fibroblast | 0.0 | 0.0 | 0.0 |
| | | | | IL-9 | | | |
| Ramos (B cell) | 0.0 | 0.0 | 0.0 | Lung fibroblast | 0.0 | 0.0 | 0.0 |
| none | | | | IL-13 | | | |
| Ramos (B cell) | 0.0 | 0.0 | 0.0 | Lung fibroblast | 0.0 | 0.0 | 0.0 |
| ionomycin | | | | IFN gamma | | | |
| B lymphocytes | 0.0 | 0.0 | 0.0 | Dermal | 0.0 | 0.0 | 0.0 |
| PWM | | | | fibroblast | | | |
| | | | | CCD1070 rest | | | |
| B lymphocytes | 0.0 | 0.0 | 0.0 | Dermal | 0.0 | 0.0 | 0.0 |
| CD40L and IL-4 | | | | fibroblast | | | |
| | | | | CCD1070 TNF | | | |
| | | | | alpha | | | |
| EOL-1 dbcAMP | 0.0 | 0.0 | 0.0 | Dermal | 0.0 | 0.0 | 0.0 |
| | | | | fibroblast | | | |
| | | | | CCD1070 IL-1 | | | |
| | | | | beta | | | |
| EOL-1 dbcAMP | 0.0 | 0.0 | 0.0 | Dermal | 0.0 | 0.0 | 0.0 |
| PMA/ionomycin | | | | fibroblast IFN | | | |
| | | | | gamma | | | |
| Dendritic cells | 0.0 | 0.0 | 0.0 | Dermal | 0.0 | 0.0 | 0.0 |
| none | | | | fibroblast IL-4 | | | |
| Dendritic cells | 5.8 | 0.0 | 0.0 | Dermal | 0.4 | 0.0 | 0.0 |
| LPS | | | | Fibroblasts rest | | | |
| Dendritic cells | 0.0 | 0.0 | 0.0 | Neutrophils | 0.0 | 0.0 | 0.0 |
| anti-CD40 | | | | TNFa + LPS | | | |
| Monocytes rest | 0.0 | 0.0 | 0.0 | Neutrophils rest | 0.0 | 0.0 | 0.0 |
| Monocytes LPS | 0.0 | 0.0 | 0.0 | Colon | 0.0 | 0.0 | 0.0 |
| Macrophages rest | 0.0 | 0.0 | 0.0 | Lung | 5.7 | 0.0 | 0.0 |
| Macrophages LPS | 0.0 | 0.0 | 0.0 | Thymus | 2.7 | 0.3 | 1.6 |
| HUVEC none | 33.4 | 14.9 | 27.5 | Kidney | 0.0 | 0.8 | 1.0 |
| HUVEC starved | 55.9 | 20.6 | 49.7 | | | | |

TABLE AIN
Panel 4D

| | Rel. Exp. (%) Ag2430, | | Rel. Exp. (%) Ag2430, |
|---|---|---|---|
| Tissue Name | Run 159506306 | Tissue Name | Run 159506306 |
| Secondary Th1 act | 0.0 | HUVEC IL-1beta | 5.9 |
| Secondary Th2 act | 0.6 | HUVEC IFN gamma | 40.3 |
| Secondary Tr1 act | 0.0 | HUVEC TNF alpha + IFN | 5.1 |
| | | gamma | |
| Secondary Th1 rest | 0.0 | HUVEC TNF alpha + IL4 | 4.0 |
| Secondary Th2 rest | 0.0 | HUVEC IL-11 | 47.3 |
| Secondary Tr1 rest | 0.0 | Lung Microvascular EC none | 22.1 |
| Primary Th1 act | 0.0 | Lung Microvascular EC | 22.5 |
| | | TNF alpha + IL-1beta | |
| Primary Th2 act | 0.0 | Microvascular Dermal EC | 20.4 |
| | | none | |
| Primary Tr1 act | 0.0 | Microsvasular Dermal EC | 3.8 |
| | | TNF alpha + IL-1beta | |
| Primary Th1 rest | 0.0 | Bronchial epithelium | 0.0 |
| | | TNF alpha + IL1beta | |
| Primary Th2 rest | 0.0 | Small airway epithelium none | 0.0 |
| Primary Tr1 rest | 0.0 | Small airway epithelium | 0.0 |
| | | TNF alpha + IL-1beta | |
| CD45RA CD4 | 0.0 | Coronery artery SMC rest | 4.2 |
| lymphocyte act | | | |
| CD45RO CD4 | 0.0 | Coronery artery SMC | 2.1 |
| lymphocyte act | | TNF alpha + IL-1beta | |
| CD8 lymphocyte act | 0.0 | Astrocytes rest | 1.1 |
| Secondary CD8 | 0.0 | Astrocytes TNF alpha + IL- | 0.9 |
| lymphocyte rest | | 1beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CCD1106 (Keratinocytes) | 0.0 |
| CD95 CH11 | | none | |
| LAK cells rest | 0.0 | CCD1106 (Keratinocytes) | 0.0 |
| | | TNF alpha + IL-1beta | |
| LAK cells IL-2 | 0.0 | Liver cirrhosis | 0.4 |
| LAK cells IL-2 + IL-12 | 0.0 | Lupus kidney | 0.2 |
| LAK cells IL-2 + IFN | 0.0 | NCI-H292 none | 0.0 |
| gamma | | | |
| LAK cells IL-2 + IL-18 | 0.0 | NCI-H292 IL-4 | 0.0 |
| LAK cells | 0.0 | NCI-H292 IL-9 | 0.0 |
| PMA/ionomycin | | | |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 85.3 |
| Two Way MLR 7 day | 0.0 | HPAEC TNF alpha + IL-1beta | 56.6 |
| PBMC rest | 0.0 | Lung fibroblast none | 0.0 |
| PBMC PWM | 0.0 | Lung fibroblast TNF alpha + | 0.0 |
| | | IL-1beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) ionomycin | 0.0 | Lung fibroblast IL-13 | 0.0 |
| B lymphocytes PWM | 0.0 | Lung fibroblast IFN gamma | 0.0 |
| B lymphocytes CD40L | 0.0 | Dermal fibroblast CCD1070 | 0.0 |
| and IL-4 | | rest | |
| EOL-1 dbcAMP | 0.0 | Dermal fibroblast CCD1070 | 0.0 |
| | | TNF alpha | |
| EOL-1 dbcAMP | 0.0 | Dermal fibroblast CCD1070 | 0.0 |
| PMA/ionomycin | | IL-1beta | |
| Dendritic cells none | 0.0 | Dermal fibroblast IFN gamma | 0.0 |
| Dendritic cells LPS | 0.0 | Dermal fibroblast IL-4 | 0.0 |
| Dendritic cells anti-CD40 | 0.0 | IBD Colitis 2 | 0.0 |
| Monocytes rest | 0.0 | IBD Crohn's | 0.0 |
| Monocytes LPS | 0.0 | Colon | 0.7 |
| Macrophages rest | 0.0 | Lung | 3.1 |
| Macrophages LPS | 0.0 | Thymus | 2.6 |
| HUVEC none | 38.7 | Kidney | 7.2 |
| HUVEC starved | 100.0 | | |

TABLE AIO
Panel CNS_1

| | Rel. Exp. (%) Ag2430, Run | | Rel. Exp. (%) Ag2430, Run |
|---|---|---|---|
| Tissue Name | 171656292 | Tissue Name | 171656292 |
| BA4 Control | 0.0 | BA17 PSP | 2.9 |
| BA4 Control2 | 19.6 | BA17 PSP2 | 0.0 |
| BA4 Alzheimer's2 | 0.0 | Sub Nigra Control | 94.0 |
| BA4 Parkinson's | 0.0 | Sub Nigra Control2 | 25.9 |
| BA4 Parkinson's2 | 17.0 | Sub Nigra Alzheimer's2 | 15.7 |
| BA4 Huntington's | 9.8 | Sub Nigra Parkinson's2 | 36.6 |
| BA4 | 0.0 | Sub Nigra Huntington's | 100.0 |
| Huntington's2 | | | |
| BA4 PSP | 0.0 | Sub Nigra | 2.8 |
| | | Huntington's2 | |
| BA4 PSP2 | 11.0 | Sub Nigra PSP2 | 29.1 |
| BA4 Depression | 19.5 | Sub Nigra Depression | 62.4 |
| BA4 Depression2 | 0.0 | Sub Nigra Depression2 | 0.0 |
| BA7 Control | 40.1 | Glob Palladus Control | 51.4 |
| BA7 Control2 | 26.1 | Glob Palladus Control2 | 5.1 |
| BA7 Alzheimer's2 | 0.0 | Glob Palladus | 21.3 |
| | | Alzheimer's | |
| BA7 Parkinson's | 0.0 | Glob Palladus | 0.0 |
| | | Alzheimer's2 | |
| BA7 Parkinson's2 | 16.6 | Glob Palladus | 0.0 |
| | | Parkinson's | |
| BA7 Huntington's | 15.2 | Glob Palladus | 31.4 |
| | | Parkinson's2 | |
| BA7 | 0.0 | Glob Palladus PSP | 0.0 |
| Huntington's2 | | | |
| BA7 PSP | 0.0 | Glob Palladus PSP2 | 4.5 |
| BA7 PSP2 | 14.7 | Glob Palladus | 44.4 |
| | | Depression | |
| BA7 Depression | 4.3 | Temp Pole Control | 4.3 |
| BA9 Control | 7.1 | Temp Pole Control2 | 9.5 |
| BA9 Control2 | 30.8 | Temp Pole Alzheimer's | 3.6 |
| BA9 Alzheimer's | 0.0 | Temp Pole | 0.0 |
| | | Alzheimer's2 | |
| BA9 Alzheimer's2 | 0.0 | Temp Pole Parkinson's | 3.0 |
| BA9 Parkinson's | 0.0 | Temp Pole Parkinson's2 | 19.8 |
| BA9 Parkinson's2 | 33.9 | Temp Pole | 10.7 |
| | | Huntington's | |
| BA9 Huntington's | 37.4 | Temp Pole PSP | 0.0 |
| BA9 | 0.0 | Temp Pole PSP2 | 1.7 |
| Huntington's2 | | | |
| BA9 PSP | 0.0 | Temp Pole Depression2 | 2.5 |
| BA9 PSP2 | 1.9 | Cing Gyr Control | 86.5 |
| BA9 Depression | 5.2 | Cing Gyr Control2 | 26.6 |
| BA9 Depression2 | 0.0 | Cing Gyr Alzheimer's | 15.3 |
| BA17 Control | 28.3 | Cing Gyr Alzheimer's2 | 13.0 |
| BA17 Control2 | 20.6 | Cing Gyr Parkinson's | 0.0 |
| BA17 | 7.0 | Cing Gyr Parkinson's2 | 49.3 |
| Alzheimer's2 | | | |
| BA17 Parkinson's | 0.0 | Cing Gyr Huntington's | 58.6 |
| BA17 | 27.2 | Cing Gyr Huntington's2 | 0.0 |
| Parkinson's2 | | | |
| BA17 | 23.7 | Cing Gyr PSP | 0.0 |
| Huntington's | | | |
| BA17 | 0.0 | Cing Gyr PSP2 | 17.0 |
| Huntington's2 | | | |
| BA17 Depression | 24.5 | Cing Gyr Depression | 49.3 |
| BA17 | 27.9 | Cing Gyr Depression2 | 19.1 |
| Depression2 | | | |

AI_comprehensive panel_v1.0 Summary: Ag4413 Highest expression of this gene is seen in a sample from a patient with Crohn's disease (CT=29.4). Moderate levels of expression are also seen in a cluster of tissues derived from patients with asthma and OA. This gene encodes a protein with homology to members of the ADAMTS family. ADAMTS proteins have been implicated in extracellular proteolysis and may play a critical role in the tissue degradation seen in arthritis and other inflammatory conditions. (Martel-Pelletier J. (2001) Best Pract Res Clin Rheumatol 15(5):805-29) Therefore, therapeutic modulation of the expression or function of this gene through the use of human monoclonal antibodies or small molecule drugs may be effective in the treatment of osteoarthritis and other autoimmune diseases.

CNS_neurodegeneration_v1.0 Summary: Ag2430/Ag4413 Two experiments with two different probe and primer sets produce results that are in excellent agreement, with highest expression in the temporal cortex of an Alzheimer's patient (CTs=30-32.7). These results confirm the expression of this gene at low levels in the brain in an independent group of individuals. This gene is found to be upregulated in the temporal cortex of Alzheimer's disease patients. Therefore, therapeutic modulation of the expression or function of this gene may decrease neuronal death and be of use in the treatment of this disease.

General_screening_panel_v1.4 Summary: Ag4413 Highest expression of this gene is seen in the cerebellum (CT=27). In addition, this gene is expressed at moderate to low levels in all regions of the CNS examined. The high levels of expression in the cerebellum suggest that this gene product may be a useful and specific target for the treatment of CNS disorders that originate in this region, such as autism and the ataxias.

Among tissues with metabolic function, this gene is expressed at moderate to low levels in adipose, pancreas, heart, and fetal skeletal muscle and liver. This expression suggests that this gene product may play a role in normal neuroendocrine and metabolic function and that disregulated expression of this gene may contribute to neuroendocrine disorders or metabolic diseases, such as obesity and diabetes.

In addition, this gene is expressed at much higher levels in fetal kidney tissue (CT=29.6) when compared to expression in the adult counterpart (CT=30.6). Thus, expression of this gene may be used to differentiate between the fetal and adult source of this tissue.

Moderate levels of expression are also seen in cell lines from brain, colon, lung, renal and melanoma cancers. Thus, expression of this gene may potentially be used as a marker of these cancers. Therapeutic modulation of this gene product may also be useful in the treatment of these cancers.

Oncology_cell_line_screening_panel_v3.2 Summary: Ag2430 Expression of the gene on this panel is limited to cerebellum and lung cancer cell lines. This is in agreement with the expression seen in Panels 1.3D and 1.4. Thus, expression of this gene could be used as a marker of cerebellar tissue and lung cancer and to differentiate these samples from other samples on this panel.

Panel 1.3D Summary: Ag2430 Expression of the gene in this panel is in agreement with expression in Panel 1.4. Highest expression is seen in the cerebellum (CT=31), with low but significant expression detected in the amygdala, hippocampus, substantia nigra and thalamus. Moderate to low levels of expression are seen in fetal skeletal muscle, adipose, and cancer cell lines derived from melanoma, breast, lung, renal, colon and brain cancers. Please see Panel 1.4 for further discussion of utility of this gene in human disease.

Panel 2D Summary: Ag2430 Highest expression of this gene is seen in lung cancer (CT=31). In addition, expression of this gene appears to be upregulated in lung, thyroid, gastric and ovarian cancer when compared to expression in the corresponding normal adjacent tissue. This protein is homologous to members of the family of ADAMTS proteins that are characterized by disintegrin, metalloproteinase and thrombospondin domains. This domain structure alone leads one to speculate that the expression of these genes in the context of cancer might play a role in the progression of the disease, as both metalloproteinases and thrombospondins have been demonstrated to be important to tumor progression. Specifically, the metalloproteinase domain may play a role in cell invasion and metastasis, and the thrombospondin domain may play a role in angiogenesis. (Masui T. Clin Cancer Res November 2001;7(11):3437-4)

Based on the expression profile of this gene and the role played by ADAMTS proteins in tumor progression, this gene in the correct context might play a role in tumor angiogeneis. Furthermore, therapeutic targeting with antibodies or small molecule drugs directed against this gene product may block the angiogenic and invasion/metastasis promoting activities of this molecule especially in those cancer types where the gene is overexpressed in the tumor compared to the normal adjacent tissue.

Panel 4.1D Summary: Ag4413/Ag7012 Three experiments with two different probe and primer set produce results that are in excellent agreement. Highest expression is seen in TNF-a and IL-1 beta treated HPAECs. This gene appears to be preferentially expressed in endothelial cells, including microvascular dermal endothelial cells, microvascular lung endothelial cells, human pulmonary aortic endothelial cells and human umbilical vein endothelial cells. Endothelial cells are known to play important roles in inflammatory responses by altering the expression of surface proteins that are involved in activation and recruiting of effector inflammatory cells. The expression of this gene in dermal microvascular endothelial cells suggests that this protein product may be involved in inflammatory responses to skin disorders, including psoriasis. Expression in lung microvascular endothelial cells suggests that the protein encoded by this gene may also be involved in lung disorders including asthma, allergies, chronic obstructive pulmonary disease, and emphysema. The protein encoded by this gene has homology to ADAMTS family of molecules suggesting that it may function as an enzyme. Based on its homology, it may contribute to the tissue destruction and remodeling processes associated with asthma, ulcerative colitis, emphysema and osteoarthritis. (Kuno K. J Biol Chem Jan. 3, 1997;272(1):556-62;) Therefore, blocking the function of the protein encoded by this gene with human nonoclonal antibody therapeutics or small molecule therapeutics may reduce or inhibit tissue destruction in the lungs, intestine, or joints due to emphysema, allergy, asthma, colitis, or osteoarthritis.

Panel 4D Summary: Ag2430 Highest expression of the gene in this panel is seen in HUVECs (CT=28). Expression in this panel is in agreement with expression in Panel 4.1D, with preferential expression seen in endothelial cells, including HPAECs, lung and dermal microvascular ECs, and a cluster of HUVEC samples. Please see Panel 4D for discussion of this gene in inflammation.

Panel CNS_{—}1 Summary: Ag2430 This panel confirms the presence of this gene in the brain. Please see Panels 1.4 and CNS_neurodegeneration for discussion of this gene in the central nervous system.

AJ. CG110242-01: Ebnerin

Expression of gene CG110242-01 was assessed using the primer-probe sets Ag1000 and Ag855, described in Tables AJA and AJB. Results of the RTQ-PCR runs are shown in Table AJC.

TABLE AJA
Probe Name Ag1000

| | | | | |
|---|---|---|---|---|
| | | | Start | SEQ ID |
| Primers | Sequences | Length | Position | No |

| | | | | |
|---|---|---|---|---|
| Forward | 5′-tgtggtggcattattaccaact-3′ | 22 | 967 | 288 |
| Probe | TET-5′-ccccacagaatgaaatgcatgaca-3′-TAMRA | 24 | 1010 | 289 |
| Reverse | 5′-atttcccacacacaagtgatgt-3′ | 22 | 1034 | 290 |

TABLE AJB

Probe Name Ag855

| | | | | |
|---|---|---|---|---|
| Primers | Sequences | Length | Position | No |

| | | | | |
|---|---|---|---|---|
| Forward | 5′-ggaaatgccagcagtatatgat-3′ | 22 | 1073 | 291 |
| Probe | TET-5′-catttgccttgatttcccacacacaa-3′-TAMRA | 26 | 1041 | 292 |
| Reverse | 5′-aatgaaatgcatgacaacatca-3′ | 22 | 1018 | 293 |

TABLE AJC
General_screening_panel_v1.5

| | Rel. Exp. (%) Ag855, Run | | Rel. Exp. (%) Ag855, Run |
|---|---|---|---|
| Tissue Name | 258465524 | Tissue Name | 258465524 |
| Adipose | 0.0 | Renal ca. TK-10 | 0.0 |
| Melanoma* | 0.0 | Bladder | 100.0 |
| Hs688(A).T | | | |
| Melanoma* Hs688(B).T | 0.0 | Gastric ca. (liver met.) | 0.0 |
| | | NCI-N87 | |
| Melanoma* M14 | 0.0 | Gastric ca. KATO III | 0.0 |
| Melanoma* LOXIMVI | 0.0 | Colon ca. SW-948 | 0.0 |
| Melanoma* SK-MEL-5 | 0.0 | Colon ca. SW480 | 0.0 |
| Squamous cell | 0.0 | Colon ca.* (SW480 met) | 0.0 |
| carcinoma SCC-4 | | SW620 | |
| Testis Pool | 23.5 | Colon ca. HT29 | 0.0 |
| Prostate ca.* (bone met) | 0.0 | Colon ca. HCT-116 | 0.0 |
| PC-3 | | | |
| Prostate Pool | 2.1 | Colon ca. CaCo-2 | 0.0 |
| Placenta | 0.7 | Colon cancer tissue | 0.0 |
| Uterus Pool | 0.4 | Colon ca. SW1116 | 0.0 |
| Ovarian ca. OVCAR-3 | 0.0 | Colon ca. Colo-205 | 0.0 |
| Ovarian ca. SK-OV-3 | 3.8 | Colon ca. SW-48 | 0.0 |
| Ovarian ca. OVCAR-4 | 0.0 | Colon Pool | 0.0 |
| Ovarian ca. OVCAR-5 | 0.0 | Small Intestine Pool | 0.9 |
| Ovarian ca. IGROV-1 | 0.6 | Stomach Pool | 0.0 |
| Ovarian ca. OVCAR-8 | 0.0 | Bone Marrow Pool | 0.0 |
| Ovary | 0.0 | Fetal Heart | 0.0 |
| Breast ca. MCF-7 | 0.0 | Heart Pool | 0.0 |
| Breast ca. MDA-MB- | 0.0 | Lymph Node Pool | 0.9 |
| 231 | | | |
| Breast ca. BT 549 | 0.0 | Fetal Skeletal Muscle | 0.0 |
| Breast ca. T47D | 0.0 | Skeletal Muscle Pool | 0.4 |
| Breast ca. MDA-N | 0.0 | Spleen Pool | 0.0 |
| Breast Pool | 0.0 | Thymus Pool | 1.0 |
| Trachea | 0.3 | CNS cancer (glio/astro) | 0.0 |
| | | U87-MG | |
| Lung | 0.0 | CNS cancer (glio/astro) U- | 0.0 |
| | | 118-MG | |
| Fetal Lung | 0.0 | CNS cancer (neuro; met) | 1.0 |
| | | SK-N-AS | |
| Lung ca. NCI-N417 | 0.0 | CNS cancer (astro) SF-539 | 0.0 |
| Lung ca. LX-1 | 0.0 | CNS cancer (astro) SNB- | 0.0 |
| | | 75 | |
| Lung ca. NCI-H146 | 0.0 | CNS cancer (glio) SNB-19 | 0.0 |
| Lung ca. SHP-77 | 0.5 | CNS cancer (glio) SF-295 | 0.0 |
| Lung ca. A549 | 0.0 | Brain (Amygdala) Pool | 2.4 |
| Lung ca. NCI-H526 | 0.0 | Brain (cerebellum) | 0.0 |
| Lung ca. NCI-H23 | 0.0 | Brain (fetal) | 5.0 |
| Lung ca. NCI-H460 | 0.0 | Brain (Hippocampus) Pool | 3.2 |
| Lung ca. HOP-62 | 0.0 | Cerebral Cortex Pool | 4.6 |
| Lung ca. NCI-H522 | 2.7 | Brain (Substantia nigra) | 9.1 |
| | | Pool | |
| Liver | 0.0 | Brain (Thalamus) Pool | 9.3 |
| Fetal Liver | 0.0 | Brain (whole) | 17.7 |
| Liver ca. HepG2 | 0.0 | Spinal Cord Pool | 2.0 |
| Kidney Pool | 0.0 | Adrenal Gland | 1.9 |
| Fetal Kidney | 0.0 | Pituitary gland Pool | 5.7 |
| Renal ca. 786-0 | 0.0 | Salivary Gland | 0.0 |
| Renal ca. A498 | 0.0 | Thyroid (female) | 0.0 |
| Renal ca. ACHN | 0.0 | Pancreatic ca. CAPAN2 | 0.0 |
| Renal ca. UO-31 | 0.0 | Pancreas Pool | 0.0 |

General_screening_panel_v1.5 Summary: Ag855 Highest expression of the CG110242-01 gene is seen in the bladder (CT=31). Thus, expression of this gene could be used to differentiate this sample from other samples on this panel and as a marker of bladder tissue. In addition, low but significant levels of expression are also seen in testis, thalamus, substantia nigra, and whole brain samples. Thus, therapeutic modulation of the expression or function of this gene may be useful in the treatment of neurologic disorders, such as Alzheimer's disease, Parkinson's disease, schizophrenia, multiple sclerosis, stroke and epilepsy.

AK. CG99598-01: Endosomal Glycoprotein

Expression of gene CG99598-01 was assessed using the primer-probe sets Ag4149 and Ag4806, described in Tables AKA and AKB. Results of the RTQ-PCR runs are shown in Table AKC.

TABLE AKA

Probe Name Ag4149

| | | | Start | SEQ ID |
|---|---|---|---|---|
| Primers | Sequences | Length | Position | No |
| Forward | 5′-ctatactctccagccccgaat-3′ | 21 | 1001 | 294 |
| Probe | TET-5′-aagcctcaggcacctccaactgct-3′-TAMRA | 24 | 1025 | 295 |
| Reverse | 5′-tgatagaagaccagctgtggaa-3′ | 22 | 1070 | 296 |

TABLE AKB

Probe Name Ag4806

| | | | Start | SEQ ID |
|---|---|---|---|---|
| Primers | Sequences | Length | Position | No |
| Forward | 5′-ctacgtggctctggatgatct-3′ | 21 | 2829 | 297 |
| Probe | TET-5′-cctgccctcagccaggttcctgt-3′-TAMRA | 23 | 2867 | 298 |
| Reverse | 5′-acacaggccagactcaaaatc-3′ | 21 | 2890 | 299 |

TABLE AKC
General_screening_panel_v1.4

| | Rel. Exp. (%) Ag4806, | | Rel. Exp. (%) Ag4806, |
|---|---|---|---|
| Tissue Name | Run 223204110 | Tissue Name | Run 223204110 |
| Adipose | 7.6 | Renal ca. TK-10 | 4.4 |
| Melanoma* | 4.6 | Bladder | 13.7 |
| Hs688(A).T | | | |
| Melanoma* | 12.7 | Gastric ca. (liver met.) | 6.6 |
| Hs688(B).T | | NCI-N87 | |
| Melanoma* M14 | 27.7 | Gastric ca. KATO III | 0.0 |
| Melanoma* LOXIMVI | 0.0 | Colon ca. SW-948 | 7.7 |
| Melanoma* SK-MEL-5 | 0.0 | Colon ca. SW480 | 9.6 |
| Squamous cell | 0.0 | Colon ca.* (SW480 met) | 7.2 |
| carcinoma SCC-4 | | SW620 | |
| Testis Pool | 0.0 | Colon ca. HT29 | 15.0 |
| Prostate ca.* (bone met) | 0.0 | Colon ca. HCT-116 | 15.2 |
| PC-3 | | | |
| Prostate Pool | 3.6 | Colon ca. CaCo-2 | 12.5 |
| Placenta | 4.7 | Colon cancer tissue | 15.8 |
| Uterus Pool | 0.0 | Colon ca. SW1116 | 16.8 |
| Ovarian ca. OVCAR-3 | 0.3 | Colon ca. Colo-205 | 13.9 |
| Ovarian ca. SK-OV-3 | 19.9 | Co1on ca. SW-48 | 0.0 |
| Ovarian ca. OVCAR-4 | 0.0 | Colon Pool | 0.0 |
| Ovarian ca. OVCAR-5 | 14.2 | Small Intestine Pool | 0.0 |
| Ovarian ca. IGROV-1 | 24.1 | Stomach Pool | 1.3 |
| Ovarian ca. OVCAR-8 | 23.2 | Bone Marrow Pool | 0.0 |
| Ovary | 0.7 | Fetal Heart | 17.0 |
| Breast ca. MCF-7 | 6.7 | Heart Pool | 4.2 |
| Breast ca. MDA-MB- | 65.1 | Lymph Node Pool | 3.5 |
| 231 | | | |
| Breast ca. BT 549 | 18.8 | Fetal Skeletal Muscle | 6.5 |
| Breast ca. T47D | 100.0 | Skeletal Muscle Pool | 11.8 |
| Breast ca. MDA-N | 8.7 | Spleen Pool | 18.7 |
| Breast Pool | 1.9 | Thymus Pool | 12.9 |
| Trachea | 0.0 | CNS cancer (glio/astro) | 25.9 |
| | | U87-MG | |
| Lung | 0.0 | CNS cancer (glio/astro) U- | 37.9 |
| | | 118-MG | |
| Fetal Lung | 19.9 | CNS cancer (neuro; met) | 9.3 |
| | | SK-N-AS | |
| Lung ca. NCI-N417 | 0.0 | CNS cancer (astro) SF-539 | 3.2 |
| Lung ca. LX-1 | 11.0 | CNS cancer (astro) SNB- | 33.4 |
| | | 75 | |
| Lung ca. NCI-H146 | 0.0 | CNS cancer (glio) SNB-19 | 11.0 |
| Lung ca. SHP-77 | 11.4 | CNS cancer (glio) SF-295 | 9.8 |
| Lung ca. A549 | 33.7 | Brain (Amygdala) Pool | 9.0 |
| Lung ca. NCI-H526 | 0.0 | Brain (cerebellum) | 22.4 |
| Lung ca. NCI-H23 | 7.0 | Brain (fetal) | 14.4 |
| Lung ca. NCI-H460 | 9.7 | Brain (Hippocampus) Pool | 8.4 |
| Lung ca. HOP-62 | 7.9 | Cerebral Cortex Pool | 0.0 |
| Lung ca. NCI-H522 | 0.0 | Brain (Substantia nigra) | 18.9 |
| | | Pool | |
| Liver | 40.1 | Brain (Thalamus) Pool | 8.2 |
| Fetal Liver | 5.1 | Brain (whole) | 3.5 |
| Liver ca. HepG2 | 16.6 | Spinal Cord Pool | 7.8 |
| Kidney Pool | 11.5 | Adrenal Gland | 4.0 |
| Fetal Kidney | 16.7 | Pituitary gland Pool | 13.9 |
| Renal ca. 786-0 | 25.7 | Salivary Gland | 0.8 |
| Renal ca. A498 | 0.0 | Thyroid (female) | 1.8 |
| Renal ca. ACHN | 0.0 | Pancreatic ca. CAPAN2 | 0.5 |
| Renal ca. UO-31 | 8.8 | Pancreas Pool | 11.7 |

General_screening_panel_v1.4 Summary: Ag4806 Expression of this gene is highest in a breast cancer cell line (CT=31.5). This gene is also expressed in breast, ovarian and colon cancer cell lines at higher levels when compared to normal tissue samples. Hence, expression of this gene might be used as a marker to identify normal tissue from cancerous tissue in these organs.

There is relatively low level of expression in most endocrine (metabolic)-related tissues except for liver. Modulation of this gene or gene-product may therefore be beneficial in treating various abnormalities related to liver function. The higher levels of expression in adult liver (CT=32.7) when compared to fetal liver suggest that expression of this gene can also be used to differentiate fetal vs adult liver tissue. Conversely, higher levels of expression in fetal lung (CT=33) when compared to adult lung (CT=40) suggest involvement of this gene in the development of the lung. Expression of this gene could also therefore be used to differentiate between fetal and adult lung tissue.

### Example D: Identification of Single Nucleotide Polymorphisms in NOVX Nucleic Acid Sequences

Variant sequences are also included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, when a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern. Examples include alteration in temporal expression, physiological response regulation, cell type expression regulation, intensity of expression, and stability of transcribed message.

SeqCalling assemblies produced by the exon linking process were selected and extended using the following criteria. Genomic clones having regions with 98% identity to all or part of the initial or extended sequence were identified by BLASTN searches using the relevant sequence to query human genomic databases. The genomic clones that resulted were selected for further analysis because this identity indicates that these clones contain the genomic locus for these SeqCalling assemblies. These sequences were analyzed for putative coding regions as well as for similarity to the known DNA and protein sequences. Programs used for these analyses include Grail, Genscan, BLAST, HMMER, FASTA, Hybrid and other relevant programs.

Some additional genomic regions may have also been identified because selected SeqCalling assemblies map to those regions. Such SeqCalling sequences may have overlapped with regions defined by homology or exon prediction. They may also be included because the location of the fragment was in the vicinity of genomic regions identified by similarity or exon prediction that had been included in the original predicted sequence. The sequence so identified was manually assembled and then may have been extended using one or more additional sequences taken from CuraGen Corporation's human SeqCalling database. SeqCalling fragments suitable for inclusion were identified by the CuraTools™ program SeqExtend or by identifying SeqCalling fragments mapping to the appropriate regions of the genomic clones analyzed.

The regions defined by the procedures described above were then manually integrated and corrected for apparent inconsistencies that may have arisen, for example, from miscalled bases in the original fragments or from discrepancies between predicted exon junctions, EST locations and regions of sequence similarity, to derive the final sequence disclosed herein. When necessary, the process to identify and analyze SeqCalling assemblies and genomic clones was reiterated to derive the full length sequence (Alderborn et al., Determination of Single Nucleotide Polymorphisms by Real-time Pyrophosphate DNA Sequencing. Genome Research. 10 (8) 1249-1265, 2000).

Variants are reported individually but any combination of all or a select subset of variants are also included as contemplated NOVX embodiments of the invention.

NOV1a SNP Data

One polymorphic variant of NOV1a has been identified and is shown in Table 42A.

TABLE 42A

| | Nucleotides | | | Amino Acids | | |
|---|---|---|---|---|---|---|
| Variant | Position | Initial | Modified | Position | Initial | Modified |
| 13379178 | 120 | A | G | 30 | Pro | Pro |

NOV2a SNP Data

One polymorphic variant of NOV2a has been identified and is shown in Table 42B.

TABLE 42B

| | Nucleotides | | | Amino Acids | | |
|---|---|---|---|---|---|---|
| Variant | Position | Initial | Modified | Position | Initial | Modified |
| 13379271 | 2850 | C | T | 909 | Asp | Asp |

NOV5a SNP Data

One polymorphic variant of NOV5a has been identified and is shown in Table 42C.

TABLE 42C

| | Nucleotides | | | Amino Acids | | |
|---|---|---|---|---|---|---|
| Variant | Position | Initial | Modified | Position | Initial | Modified |
| 13379181 | 1001 | G | C | Silent | N/A | N/A |

NOV6a SNP Data

Seven polymorphic variant of NOV6a have been identified and is shown in Table 42D.

TABLE 42D

| | Nucleotides | | | Amino Acids | | |
|---|---|---|---|---|---|---|
| Variant | Position | Initial | Modified | Position | Initial | Modified |
| 13379177 | 1599 | C | T | Silent | N/A | N/A |
| 13379176 | 1665 | G | T | Silent | N/A | N/A |
| 13379175 | 1673 | G | A | Silent | N/A | N/A |
| 13379174 | 1732 | G | A | Silent | N/A | N/A |
| 13379173 | 1791 | G | T | Silent | N/A | N/A |
| 13379172 | 1795 | G | A | Silent | N/A | N/A |
| 13379171 | 1876 | T | C | Silent | N/A | N/A |

NOV9a SNP Data

Two polymorphic variant of NOV9a have been identified and are shown in Table 42E.

TABLE 42E

| | Nucleotides | | | Amino Acids | | |
|---|---|---|---|---|---|---|
| Variant | Position | Initial | Modified | Position | Initial | Modified |
| 13379179 | 191 | A | G | 59 | Thr | Thr |
| 13379180 | 360 | G | C | 116 | Val | Leu |

NOV9b SNP Data

Three polymorphic variant of NOV9b have been identified and are shown in Table 42F.

TABLE 42F

| | Nucleotides | | | Amino Acids | | |
|---|---|---|---|---|---|---|
| | Base Position | | | Base Position | | |
| Variant No. | of SNP | Wild-type | Variant | of SNP | Wild-type | Variant |
| 13379251 | 971 | A | G | 315 | Ala | Ala |
| 13379250 | 1226 | C | T | 400 | Gly | Gly |
| 13379249 | 1712 | C | T | 562 | Tyr | Tyr |

NOV10a SNP Data

Eight polymorphic variant of NOV10a have been identified and are shown in Table 42G.

TABLE 42G

| | Nucleotides | | | Amino Acids | | |
|---|---|---|---|---|---|---|
| Variant | Position | Initial | Modified | Position | Initial | Modified |
| 13376858 | 229 | A | G | 74 | Glu | Gly |
| 13376859 | 258 | G | A | 84 | Ala | Thr |
| 13376860 | 286 | A | G | 93 | Gln | Arg |
| 13376861 | 296 | T | C | 96 | Ala | Ala |
| 13376862 | 305 | A | G | 99 | Gln | Gln |
| 13376863 | 312 | A | G | 102 | Thr | Ala |
| 13376864 | 348 | A | G | 114 | Arg | Gly |
| 13376866 | 404 | G | A | 132 | Met | Ile |

NOV11a SNP Data

Two polymorphic variant of NOV11a have been identified and are shown in Table 42H.

TABLE 42H

| | Nucleotides | | | Amino Acids | | |
|---|---|---|---|---|---|---|
| Variant | Position | Initial | Modified | Position | Initial | Modified |
| 13379186 | 346 | T | C | 96 | Ser | Ser |
| 13379187 | 539 | A | G | 161 | Ile | Val |

NOV15a SNP Data

Three polymorphic variant of NOV15a have been identified and are shown in Table 42I.

TABLE 42I

| | Nucleotides | | | Amino Acids | | |
|---|---|---|---|---|---|---|
| Variant | Position | Initial | Modified | Position | Initial | Modified |
| 13377489 | 243 | T | C | 71 | Tyr | Tyr |
| 13377488 | 348 | C | T | 106 | Ile | Ile |
| 13377487 | 723 | T | G | 231 | Thr | Thr |

NOV16a SNP Data

One polymorphic variant of NOV16a has been identified and is shown in Table 42J.

TABLE 42J

| | Nucleotides | | | Amino Acids | | |
|---|---|---|---|---|---|---|
| Variant | Position | Initial | Modified | Position | Initial | Modified |
| 13379185 | 1258 | A | G | 415 | Ser | Ser |

NOV17a SNP Data

One polymorphic variant of NOV17a has been identified and is shown in Table 42K.

TABLE 42K

| | Nucleotides | | | Amino Acids | | |
|---|---|---|---|---|---|---|
| Variant | Position | Initial | Modified | Position | Initial | Modified |
| 13379184 | 229 | A | G | 76 | Met | Val |

NOV19a SNP Data

Two polymorphic variants of NOV19a have been identified and are shown in Table 42L.

TABLE 42L

| | Nucleotides | | | Amino Acids | | |
|---|---|---|---|---|---|---|
| Variant | Position | Initial | Modified | Position | Initial | Modified |
| 13379188 | 3620 | T | C | 1187 | Val | Ala |
| 13379189 | 3725 | G | A | Silent | N/A | N/A |

NOV27a SNP Data

One polymorphic variant of NOV27a has been identified and is shown in Table 42M.

TABLE 42M

| | Nucleotides | | | Amino Acids | | |
|---|---|---|---|---|---|---|
| Variant | Position | Initial | Modified | Position | Initial | Modified |
| 13379191 | 2068 | T | C | 364 | Pro | Pro |

NOV29a SNP Data

One polymorphic variant of NOV29a has been identified and is shown in Table 42N.

TABLE 42N

| | Nucleotides | | | Amino Acids | | |
|---|---|---|---|---|---|---|
| Variant | Position | Initial | Modified | Position | Initial | Modified |
| 13379190 | 113 | C | T | 25 | Pro | Leu |

NOV31a SNP Data

One polymorphic variant of NOV31a has been identified and is shown in Table 42O.

TABLE 42O

| | Nucleotides | | | Amino Acids | | |
|---|---|---|---|---|---|---|
| Variant | Position | Initial | Modified | Position | Initial | Modified |
| 13379285 | 923 | C | T | 307 | Phe | Phe |

NOV35a SNP Data

One polymorphic variant of NOV35a has been identified and is shown in Table 42P.

TABLE 42P

| | Nucleotides | | | Amino Acids | | |
|---|---|---|---|---|---|---|
| Variant | Position | Initial | Modified | Position | Initial | Modified |
| 13378787 | 1271 | C | T | 424 | Ala | Val |

NOV39a SNP Data

Five polymorphic variant of NOV39a have been identified and are shown in Table 42Q.

TABLE 42Q

| | Nucleotides | | | Amino Acids | | |
|---|---|---|---|---|---|---|
| Variant | Position | Initial | Modified | Position | Initial | Modified |
| 13379193 | 655 | T | C | 191 | Tyr | His |
| 13379196 | 861 | A | G | 259 | Lys | Lys |
| 13379195 | 871 | A | G | 263 | Lys | Glu |
| 13374732 | 1425 | T | C | 447 | Asp | Asp |
| 13379194 | 1960 | T | A | 626 | Leu | Ile |

### Example E: Potential Role(s) of CG102615-01 in Tumorgenesis

The NOV13a gene (CG102615-01) is known to mediate chloride flow, affecting the membrane potential of the cell. Changes in membrane potential can affect tumor cell and associated smooth muscle cells (therefore tumor-induced vasculature) growth and motility. In this respect, the strong expression in fetal muscle is an indication of a role for NOV13a in muscle growth/development.

Therapeutic targeting of NOV13a with a human monoclonal antibody is anticipated to limit or block the extent of tumor cell growth and motility and tumor associated angiogenesis, preferably in breast, ovarian bladder, lung tumors.

SAGE data is present for NOV13a in Table 43.

TABLE 43
NOV13a SAGE data
Hs 301350: FXYD domain-containing ion transport regulator 3
SAGE library data and reliable tag summary
Reliable tags found in SAGE libraries

| | | | |
|---|---|---|---|
| | Tags per | Tag | |
| Library name | million | counts | Total tags |

AACCGAAAAA

| | | | |
|---|---|---|---|
| SAGE Caco 2 | 16 | 1 | 61601 |
| SAGE Chen LNCaP | 32 | 2 | 62267 |
| SAGE Chen LNCaP no-DHT | 15 | 1 | 64631 |
| SAGE Chen Tumor Pr | 14 | 1 | 68384 |
| SAGE CAPAN1 | 52 | 2 | 37926 |
| SAGE Duke GBM H1110 | 14 | 1 | 70061 |
| SAGE SW837 | 16 | 1 | 60986 |
| SAGE PR317 normal prostate | 16 | 1 | 59419 |
| SAGE PR317 prostate tumor | 46 | 3 | 65109 |
| SAGE pooled GBM | 16 | 1 | 61841 |
| SAGE NHA(5th) | 19 | 1 | 52196 |
| SAGE NC1 | 19 | 1 | 50115 |
| SAGE NC2 | 141 | 7 | 49552 |
| SAGE Panc 91-16113 | 88 | 3 | 33941 |
| SAGE Tu102 | 34 | 2 | 57636 |
| SAGE Tu98 | 61 | 3 | 49005 |
| SAGE SciencePark MCF7 | 163 | 10 | 61079 |
| Control 0 h | | | |
| SAGE SciencePark MCF7 | 33 | 2 | 59978 |
| estradiol 3 h | | | |
| SAGE 95-259 | 25 | 1 | 39473 |
| SAGE 95-260 | 22 | 1 | 45179 |
| SAGE 95-348 | 33 | 2 | 60484 |
| SAGE Medullo 3871 | 115 | 5 | 43274 |
| SAGE MouseP8 PGCP | 16 | 1 | 61240 |
| SAGE MDA453 | 52 | 1 | 18924 |
| SAGE Duke HMVEC + VEGF | 17 | 1 | 57928 |
| SAGE DCIS | 218 | 9 | 41230 |
| SAGE OVT-8 | 29 | 1 | 33575 |
| SAGE DCIS 2 | 34 | 1 | 28888 |

GCAGGGCCTC

| | | | |
|---|---|---|---|
| SAGE Caco 2 | 16 | 1 | 61601 |
| SAGE Chen LNCaP | 208 | 13 | 62267 |
| SAGE Chen LNCaP no-DHT | 278 | 18 | 64631 |
| SAGE Chen Normal Pr | 196 | 13 | 66193 |
| SAGE Chen Tumor Pr | 102 | 7 | 68384 |
| SAGE CAPAN1 | 632 | 24 | 37926 |
| SAGE CAPAN2 | 473 | 11 | 23222 |
| SAGE SW837 | 196 | 12 | 60986 |
| SAGE CPDR LNCaP-C | 48 | 2 | 41590 |
| SAGE PR317 normal prostate | 521 | 31 | 59419 |
| SAGE PR317 prostate tumor | 2042 | 133 | 65109 |
| SAGE NC1 | 2554 | 128 | 50115 |
| SAGE NC2 | 3329 | 165 | 49552 |
| SAGE Panc 91-16113 | 824 | 28 | 33941 |
| SAGE Panc 96-6252 | 111 | 4 | 35745 |
| SAGE OV1063-3 | 154 | 6 | 38938 |
| SAGE Tu102 | 884 | 51 | 57636 |
| SAGE Tu98 | 938 | 46 | 49005 |
| SAGE SciencePark MCF7 | 677 | 4 | 5903 |
| control 3 h | | | |
| SAGE SciencePark MCF7 | 343 | 21 | 61079 |
| Control 0 h | | | |
| SAGE ScincePark MCF7 | 233 | 14 | 59978 |
| estradiol 3 h | | | |
| SAGE SciencePark MCF7 | 215 | 13 | 60435 |
| estradiol 10 h | | | |
| SAGE lacZ | 377 | 7 | 18528 |
| SAGE PTEN | 213 | 2 | 9380 |
| SAGE 95-347 | 431 | 29 | 67240 |
| SAGE 95-259 | 608 | 24 | 39473 |
| SAGE 95-260 | 420 | 19 | 45179 |
| SAGE 95-348 | 942 | 57 | 60484 |
| SAGE PrCA-1 | 109 | 1 | 9105 |
| SAGE normal prostate | 152 | 2 | 13148 |
| SAGE LNCaP | 132 | 3 | 22637 |
| SAGE OVT-6 | 23 | 1 | 42336 |
| SAGE H1126 | 57 | 1 | 17501 |
| SAGE OVT-7 | 200 | 11 | 54914 |
| SAGE MDA453 | 264 | 5 | 18924 |
| SAGE SKBR3 | 735 | 6 | 8153 |
| SAGE mammary epithelium | 386 | 19 | 49167 |
| SAGE DCIS | 2328 | 96 | 41230 |
| SAGE normal cerebellum | 22 | 1 | 44421 |
| SAGE OVT-8 | 59 | 2 | 33575 |
| SAGE Duke 40N | 280 | 2 | 7142 |
| SAGE Duke 48N | 82 | 1 | 12091 |
| SAGE Duke post crisis | 13 | 1 | 71792 |
| fibroblasts | | | |
| SAGE DCIS 2 | 1557 | 45 | 28888 |
| SAGE Br N | 532 | 20 | 37558 |
| SAGE A+ | 130 | 4 | 30551 |

### OTHER EMBODIMENTS

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. The claims presented are representative of the inventions disclosed herein. Other, unclaimed inventions are also contemplated. Applicants reserve the right to pursue such inventions in later claims.

What is claimed is:

1. An isolated polypeptide comprising the mature form of an amino acid sequence selected from the group consisting of SEQ ID NO:2n, wherein n is an integer between 1 and 73.

2. An isolated polypeptide comprising an amino acid sequence selected from the group consisting of SEQ ID NO:2n, wherein n is an integer between 1 and 73.

3. An isolated polypeptide comprising an amino acid sequence which is at least 95% identical to an amino acid sequence selected from the group consisting of SEQ ID NO:2n, wherein n is an integer between 1 and 73.

4. An isolated polypeptide, wherein the polypeptide comprises an amino acid sequence comprising one or more conservative substitutions in the amino acid sequence selected from the group consisting of SEQ ID NO:2n, wherein n is an integer between 1 and 73.

5. The polypeptide of claim 1 wherein said polypeptide is naturally occurring.

6. A composition comprising the polypeptide of claim 1 and a carrier.

7. A kit comprising, in one or more containers, the composition of claim 6.

8. The use of a therapeutic in the manufacture of a medicament for treating a syndrome associated with a human disease, the disease selected from a pathology associated with the polypeptide of claim 1, wherein the therapeutic comprises the polypeptide of claim 1.

9. A method for determining the presence or amount of the polypeptide of claim 1 in a sample, the method comprising:
(a) providing said sample;
(b) introducing said sample to an antibody that binds immunospecifically to the polypeptide; and
(c) determining the presence or amount of antibody bound to said polypeptide, thereby determining the presence or amount of polypeptide in said sample.

10. A method for determining the presence of or predisposition to a disease associated with altered levels of expression of the polypeptide of claim 1 in a first mammalian subject, the method comprising:
a) measuring the level of expression of the polypeptide in a sample from the first mammalian subject; and
b) comparing the expression of said polypeptide in the sample of step (a) to the expression of the polypeptide present in a control sample from a second mammalian subject known not to have, or not to be predisposed to, said disease,
wherein an alteration in the level of expression of the polypeptide in the first subject as compared to the control sample indicates the presence of or predisposition to said disease.

11. A method of identifying an agent that binds to the polypeptide of claim 1, the method comprising:
(a) introducing said polypeptide to said agent; and
(b) determining whether said agent binds to said polypeptide.

12. The method of claim 11 wherein the agent is a cellular receptor or a downstream effector.

13. A method for identifying a potential therapeutic agent for use in treatment of a pathology, wherein the pathology is related to aberrant expression or aberrant physiological interactions of the polypeptide of claim 1, the method comprising:
(a) providing a cell expressing the polypeptide of claim 1 and having a property or function ascribable to the polypeptide;
(b) contacting the cell with a composition comprising a candidate substance; and
(c) determining whether the substance alters the property or function ascribable to the polypeptide;
whereby, if an alteration observed in the presence of the substance is not observed when the cell is contacted with a composition in the absence of the substance, the substance is identified as a potential therapeutic agent.

14. A method for screening for a modulator of activity of or of latency or predisposition to a pathology associated with the polypeptide of claim 1, said method comprising:
(a) administering a test compound to a test animal at increased risk for a pathology associated with the polypeptide of claim 1, wherein said test animal recombinantly expresses the polypeptide of claim 1;
(b) measuring the activity of said polypeptide in said test animal after administering the compound of step (a); and
(c) comparing the activity of said polypeptide in said test animal with the activity of said polypeptide in a control animal not administered said polypeptide, wherein a change in the activity of said polypeptide in said test animal relative to said control animal indicates the test compound is a modulator activity of or latency or predisposition to, a pathology associated with the polypeptide of claim 1.

15. The method of claim 14, wherein said test animal is a recombinant test animal that expresses a test protein transgene or expresses said transgene under the control of a promoter at an increased level relative to a wild-type test animal, and wherein said promoter is not the native gene promoter of said transgene.

16. A method for modulating the activity of the polypeptide of claim 1, the method comprising contacting a cell sample expressing the polypeptide of claim 1 with a compound that binds to said polypeptide in an amount sufficient to modulate the activity of the polypeptide.

17. A method of treating or preventing a pathology associated with the polypeptide of claim 1, the method comprising administering the polypeptide of claim 1 to a subject in which such treatment or prevention is desired in an amount sufficient to treat or prevent the pathology in the subject.

18. The method of claim 17, wherein the subject is a human.

19. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising the amino acid sequence selected from the group consisting of SEQ ID NO:2n, wherein n is an integer between 1 and 73 or a biologically active fragment thereof.

20. An isolated nucleic acid molecule comprising a nucleic acid sequence selected from the group consisting of SEQ ID NO:2n−1, wherein n is an integer between 1 and 73.

21. The nucleic acid molecule of claim 20, wherein the nucleic acid molecule is naturally occurring.

22. A nucleic acid molecule, wherein the nucleic acid molecule differs by a single nucleotide from a nucleic acid sequence selected from the group consisting of SEQ ID NO: 2n−1, wherein n is an integer between 1 and 73.

23. An isolated nucleic acid molecule encoding the mature form of a polypeptide having an amino acid sequence selected from the group consisting of SEQ ID NO:2n, wherein n is an integer between 1 and 73.

24. An isolated nucleic acid molecule comprising a nucleic acid selected from the group consisting of 2n−1, wherein n is an integer between 1 and 73.

25. The nucleic acid molecule of claim 20, wherein said nucleic acid molecule hybridizes under stringent conditions to the nucleotide sequence selected from the group consisting of SEQ ID NO: 2n−1, wherein n is an integer between 1 and 73, or a complement of said nucleotide sequence.

26. A vector comprising the nucleic acid molecule of claim 20.

27. The vector of claim 26, further comprising a promoter operably linked to said nucleic acid molecule.

28. A cell comprising the vector of claim 26.

29. An antibody that immunospecifically binds to the polypeptide of claim 1.

30. The antibody of claim 29, wherein the antibody is a monoclonal antibody.

31. The antibody of claim 29, wherein the antibody is a humanized antibody.

32. The antibody of claim 29, wherein the antibody is a fully human antibody.

33. The antibody of claim 29, wherein the dissociation constant for the binding of the polypeptide to the antibody is less than 1×10⁻⁹ M.

34. The antibody of claim 29, wherein the antibody neutralizes an activity of the polypeptide.

35. A method of treating or preventing a NOVX-associated disorder, the method comprising administering to a subject in which such treatment or prevention is desired the antibody of claim 29 in an amount sufficient to treat or prevent the pathology in the subject.

36. The method of claim 35, wherein the subject is human.

37. A method for determining the presence or amount of the nucleic acid molecule of claim 20 in a sample, the method comprising:
(a) providing said sample;
(b) introducing said sample to a probe that binds to said nucleic acid molecule; and
(c) determining the presence or amount of said probe bound to said nucleic acid molecule,
thereby determining the presence or amount of the nucleic acid molecule in said sample.

38. The method of claim 37 wherein presence or amount of the nucleic acid molecule is used as a marker for cell or tissue type.

39. The method of claim 38 wherein the cell or tissue type is cancerous.

40. A method for determining the presence of or predisposition to a disease associated with altered levels of expression of the nucleic acid molecule of claim 20 in a first mammalian subject, the method comprising:
a) measuring the level of expression of the nucleic acid in a sample from the first mammalian subject; and
b) comparing the level of expression of said nucleic acid in the sample of step (a) to the level of expression of the nucleic acid present in a control sample from a second mammalian subject known not to have or not be predisposed to, the disease;
wherein an alteration in the level of expression of the nucleic acid in the first subject as compared to the control sample indicates the presence of or predisposition to the disease.

41. A method of producing the polypeptide of claim 1, the method comprising culturing a cell under conditions that lead to expression of the polypeptide, wherein said cell comprises a vector comprising an isolated nucleic acid molecule comprising a nucleic acid sequence selected from the group consisting of SEQ ID NO:2n−1, wherein n is an integer between 1 and 73.

42. The method of claim 41 wherein the cell is a bacterial cell.

43. The method of claim 41 wherein the cell is an insect cell.

44. The method of claim 41 wherein the cell is a yeast cell.

45. The method of claim 41 wherein the cell is a mammalian cell.

46. A method of producing the polypeptide of claim 2, the method comprising culturing a cell under conditions that lead to expression of the polypeptide, wherein said cell comprises a vector comprising an isolated nucleic acid molecule comprising a nucleic acid sequence selected from the group consisting of SEQ ID NO:2n−1, wherein n is an integer between 1 and 73.

47. The method of claim 46 wherein the cell is a bacterial cell.

48. The method of claim 46 wherein the cell is an insect cell.

49. The method of claim 46 wherein the cell is a yeast cell.

50. The method of claim 46 wherein the cell is a mammalian cell.
